SEQUENCE LISTING (247 sequences)

SEQ ID NO: 1 - protein, 95 aa, Mycobacterium tuberculosis
SQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 2 - protein, 90 aa, Mycobacterium tuberculosis
PVGGQSSFYSDWYSPACGKAGCQTYKWETFLTSELPQWLSANRAVKPTGSAAIGLSMAGSSAMILAAYHPQQFIYAGSLSALLDPSQGMG

SEQ ID NO: 3 - protein, 248 aa, Mycobacterium tuberculosis
VDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHM

SEQ ID NO: 4 - protein, 93 aa, Mycobacterium tuberculosis
TINYQFGDVDAHGAMIRAQAGSLEAEHQAIISDVLTASDFWGGAGSAACQGFITQLGRNFQVIYEQANAHGQKVQAAGNNMAQTDSAVGSSWA

SEQ ID NO: 5 - protein, 97 aa, Mycobacterium tuberculosis
TSRFMTDPHAMRDMAGRFEVHAQTVEDEARRMWASAQNISGAGWSGMAEATSLDTMTQMNQAFRNIVNMLHGVRDGLVRDANNYEQQEQASQQILSS

SEQ ID NO: 6 - protein, 99 aa, Mycobacterium tuberculosis
AEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 7 - protein, 94 aa, Mycobacterium tuberculosis
TEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 8 - protein, 15 aa, Mycobacterium tuberculosis
MSFVTTQPEALAAAA

SEQ ID NO: 9 - protein, 15 aa, Mycobacterium tuberculosis
AQIYQAVSAQAAAIH

SEQ ID NO: 10 - protein, 15 aa, Mycobacterium tuberculosis
PSPSMGRDIKVQFQS

SEQ ID NO: 11 - protein, 15 aa, Mycobacterium tuberculosis
GINTIPIAINEAEYV

SEQ ID NO: 12 - protein, 15 aa, Mycobacterium tuberculosis
AAFQGAHARFVAAAA

SEQ ID NO: 13 - protein, 15 aa, Mycobacterium tuberculosis
AGWLAFFRDLVARGL

SEQ ID NO: 14 - protein, 15 aa, Mycobacterium tuberculosis
ASIIRLVGAVLAEQH

SEQ ID NO: 15 - protein, 15 aa, Mycobacterium tuberculosis
MHVSFVMAYPEMLAA

SEQ ID NO: 16 - protein, 15 aa, Mycobacterium tuberculosis
AYGSFVRTVSLPVGA

SEQ ID NO: 17 - protein, 15 aa, Mycobacterium tuberculosis
LENDNQLLYNYPGAL

SEQ ID NO: 18 - protein, 1046 aa, synthetic construct
AEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGAQIYQAVSAQAAAIHGPGPGPSPSMGRDIKVQFQSGPGPGGINTIPIAINEAEYVGPGPGAAFQGAHARFVAAAAGPGPGAGWLAFFRDLVARGLGPGPGASIIRLVGAVLAEQHGPGPGMSFVTTQPEALAAAAGPGPGMHVSFVMAYPEMLAAGPGPGAYGSFVRTVSLPVGAGPGPGLENDNQLLYNYPGAL

SEQ ID NO: 19 - protein, 1046 aa, synthetic construct
AEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGAQIYQAVSAQAAAIHGPGPGPSPSMGRDIKVQFQSGPGPGGINTIPIAINEAEYVGPGPGAAFQGAHARFVAAAAGPGPGAGWLAFFRDLVARGLGPGPGASIIRLVGAVLAEQHGPGPGMSFVTTQPEALAAAAGPGPGMHVSFVMAYPEMLAAGPGPGAYGSFVRTVSLPVGAGPGPGLENDNQLLYNYPGAL

SEQ ID NO: 20 - protein, 1046 aa, synthetic construct
AEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGAQIYQAVSAQAAAIHGPGPGPSPSMGRDIKVQFQSGPGPGGINTIPIAINEAEYVGPGPGAAFQGAHARFVAAAAGPGPGAGWLAFFRDLVARGLGPGPGASIIRLVGAVLAEQHGPGPGMSFVTTQPEALAAAAGPGPGMHVSFVMAYPEMLAAGPGPGAYGSFVRTVSLPVGAGPGPGLENDNQLLYNYPGAL

SEQ ID NO: 21 - protein, 28 aa, Homo sapiens
MAAPGSARRPLLLLLLLLLLGLMHCASA

SEQ ID NO: 22 - protein, 35 aa, Homo sapiens
LIPIAVGGALAGLVLIVLIAYLVGRKRSHAGYQTI

SEQ ID NO: 23 - protein, 24 aa, Homo sapiens
MLVMAPRTVLLLLSAALALTETWA

SEQ ID NO: 24 - protein, 26 aa, Homo sapiens
MRVTAPRTLILLLSGALALTETWAGS

SEQ ID NO: 25 - protein, 53 aa, Homo sapiens [sequence omitted]

SEQ ID NO: 26 - protein, 55 aa, Homo sapiens
IVGIVAGLAVLAVVVIGAVVATVMCRRKSSGGKGGSYSQAASSDSAQGSDVSLTA

SEQ ID NO: 27 - protein, 29 aa, Homo sapiens
MVCLKLPGGSCMTALTVTLMVLSSPLALS

SEQ ID NO: 28 - protein, 39 aa, Homo sapiens
MLSGVGGFVLGLLFLGAGLFIYFRNQKGHSGLQPTGFLS

SEQ ID NO: 29 - protein, 25 aa, Homo sapiens
MAISGVPVLGFFIIAVLMSAQESWA

SEQ ID NO: 30 - protein, 22 aa, synthetic construct
MDAMKRGLCCVLLLCGAVFVSA

SEQ ID NO: 31 - protein, 98 aa, synthetic construct
SFVMAYPEMLAAAADTLQSIGATTVASNAAAAAPTTGVVPPAADEVSALTAAHFAAHAAMYQSVSARAAAIHDQFVATLASSASSYAATEVANAAAAS

SEQ ID NO: 32 - RNA, 628 nt, Homo sapiens
  source 1..628: mol_type = genomic RNA
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 33 - protein, 1109 aa, synthetic construct
MAAPGSARRPLLLLLLLLLLGLMHCASAAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGAQIYQAVSAQAAAIHGPGPGPSPSMGRDIKVQFQSGPGPGGINTIPIAINEAEYVGPGPGAAFQGAHARFVAAAAGPGPGAGWLAFFRDLVARGLGPGPGASIIRLVGAVLAEQHGPGPGMSFVTTQPEALAAAAGPGPGMHVSFVMAYPEMLAAGPGPGAYGSFVRTVSLPVGAGPGPGLENDNQLLYNYPGALLIPIAVGGALAGLVLIVLIAYLVGRKRSHAGYQTI

SEQ ID NO: 34 - RNA, 3523 nt, synthetic construct
  source 1..3523: mol_type = other RNA
acatttgcttctgacacaactgtgttcactagcaacctcaaacagccaccatggccgctcctggctccgcgcgcagaccgttgctccttctcttactactgctgctgttgggacttatgcactgcgcttccgccgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggaccgggacccggcaccgaacaacagtggaacttcgccggtattgaggccgccgcctcggcgattcagggaaatgtcacttccattcattccctgctcgacgagggaaaacagagcctcaccaagcttgcagctgcctggggtggttcgggctccgaagcctaccagggggtgcagcagaagtgggatgcgactgccaccgaactgaacaacgcgctccaaaatctggctcggaccattagcgaagctggtcaggctatggcgtccaccgagggcaacgtgaccggaatgttcgcaggcccgggtcccggatcccaaatcatgtataactacccagcaatgctgggccatgccggcgacatggcggggtacgccggcaccttacagtctctaggcgctgaaatcgccgtcgaacaagccgccctgcaatccgcctggcaaggagataccggaattacctatcaggcttggcaggcgcaatggaaccaggccatggaggacctcgtgcgggcctaccacgccatgtcaagcacccacgaagccaacactatggccatgatggctagagatactgcagaggctgccaagtggggaggcggcccgggacccggacctgtgggaggccagtccagcttctacagcgactggtacagcccagcctgcggaaaggccggctgccaaacatacaagtgggaaaccttccttacctccgaactcccccagtggctttccgcgaaccgggccgtgaagccgactggatcagcagccatcggccttagcatggcgggctcgtcggccatgattctggccgcataccatcctcagcaattcatctacgccggatcgctttccgccctgctggacccctcacagggtatgggtggacctgggcccggcgtggacttcggagccctgccacctgaaatcaactccgcccggatgtacgcaggacctggatccgccagcctggtggcggccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcggccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcggaccggggccgggaaccataaactaccagtttggcgacgtcgacgcccacggagccatgatcagggcgcaggccgggtcgctggaagcagaacaccaggccatcatctccgatgtgctgaccgcctccgacttttggggaggagccggttcggctgcctgccaagggttcatcacacaattgggaaggaacttccaggtcatctacgagcaggccaatgcacacggtcaaaaggtccaagcggcgggcaacaacatggcccagactgactcggccgtgggcagcagttgggccggaccgggccctggggcccagatctaccaagcagtgtcggcccaagccgcagcgatccatggaccaggaccgggaccgtccccgagcatgggccgggatattaaggttcagttccagtccggacccggcccggggggtattaacaccattccgattgccattaatgaggcagaatacgtcggccctggtcccggcgccgcgttccaaggagcccacgcacgattcgtggcggcagctgcgggacccgggcctggtgccggttggctggcttttttccgcgacctggtggccagaggacttggacccggtccgggcgccagcattatcaggctggtcggggccgtgctggcggaacagcatggacctggcccgggaatgagcttcgtgaccacccaacctgaggcgcttgccgcggcggcaggccctggacccgggatgcatgtgtcttttgtgatggcctacccggaaatgctggccgcgggtccgggccccggagcctacggatcattcgtgcgcactgtgtccctccccgtgggagccggacctggaccggggttggagaatgataaccagctgctctacaactatccgggggccctcctgatccccatcgccgtgggcggggccctggccgggctggttctgatcgtcctgattgcatacctggtcggacggaagcgctctcacgccggctaccaaaccatctgatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 35 - protein, 1109 aa, synthetic construct
MAAPGSARRPLLLLLLLLLLGLMHCASAAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGAQIYQAVSAQAAAIHGPGPGPSPSMGRDIKVQFQSGPGPGGINTIPIAINEAEYVGPGPGAAFQGAHARFVAAAAGPGPGAGWLAFFRDLVARGLGPGPGASIIRLVGAVLAEQHGPGPGMSFVTTQPEALAAAAGPGPGMHVSFVMAYPEMLAAGPGPGAYGSFVRTVSLPVGAGPGPGLENDNQLLYNYPGALLIPIAVGGALAGLVLIVLIAYLVGRKRSHAGYQTI

SEQ ID NO: 36 - RNA, 3523 nt, synthetic construct
  source 1..3523: mol_type = other RNA
acatttgcttctgacacaactgtgttcactagcaacctcaaacagccaccatggccgctccaggctcagcccgcagacccttgctcctcctgttactactgctgctgctcgggttgatgcactgcgcgtccgccgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcggacctggccccggtaccatcaactaccagttcggcgacgtggacgcacacggcgccatgatcagggcccaggctgggtcgctagaagccgagcaccaggctatcatttctgatgtcctcaccgcgtcagatttttgggggggggcggggtcagccgcctgccagggattcataacccagctcggccggaacttccaggtcatctacgaacaggccaacgctcacggtcagaaggtgcaggcggccggtaacaacatggcccagactgactccgccgtggggtcaagctgggccgggccggggcctggatcacaaatcatgtacaactacccggcaatgctgggccacgctggagacatggccggctacgccggcacgctgcagtcactcggtgccgagatcgccgtcgagcaggcggcactgcagagcgcttggcagggagacactggcattacctaccaagcgtggcaggctcagtggaatcaagctatggaagatctggtccgcgcgtaccacgccatgtcctccactcacgaagccaacaccatggctatgatggccagggacactgccgaggcagccaaatgggggggaggtcccggccctggccctgtcggggggcagtcatccttctactccgactggtactcccccgcctgcggaaaggccggctgtcagacctataagtgggaaaccttcctgacctccgaactcccgcagtggctctcggcgaatcgggcagtgaagcccaccgggtcggcggctattggactgtccatggccggttccagcgcgatgatcctggccgcctatcacccccagcaattcatctacgctggctccctgtccgccctgcttgacccatcgcaggggatgggtggcccgggaccgggagtggacttcggggccttgccacccgaaatcaactctgccagaatgtacgccgggcctggatccgcgtcccttgtagccgcggcccagatgtgggactccgtggcctcagacctgttctccgccgcgtcggcgtttcagtccgtggtgtgggggctgactgtgggcagctggatcggaagctccgccggactgatggtcgccgccgccagcccgtacgtggcttggatgagcgtcactgccgggcaggccgagctcactgccgcacaggtccgcgtcgcggccgcagcctacgaaaccgcatacggactgacagtgccccccccggtgatcgccgagaaccgggcggaactgatgatccttattgccaccaacctcttgggacaaaatacccccgccattgcggtgaacgaggcggagtatggagagatgtgggcccaggacgccgcggccatgtttgggtacgccgccgcaaccgcgactgccaccgccacgctgctgccgttcgaggaggcccccgaaatgacctcggcaggagggctgctcgagcaggccgccgctgtggaagaggcatccgatactgccgctgccaaccaactcatgaacaatgtgcctcaggccctgcagcagctggcccagcccacccagggaaccaccccgagctcgaagcttggagggctgtggaaaacggtgtcaccgcaccggtcccccatttccaacatggtgtcgatggccaacaaccatatgggaccgggccctggggcccagatctaccaagcagtgtcggcccaagccgcagcgatccatggaccaggaccgggaccgtccccgagcatgggccgggatattaaggttcagttccagtccggacccggcccggggggtattaacaccattccgattgccattaatgaggcagaatacgtcggccctggtcccggcgccgcgttccaaggagcccacgcacgattcgtggcggcagctgcgggacccgggcctggtgccggttggctggcttttttccgcgacctggtggccagaggacttggacccggtccgggcgccagcattatcaggctggtcggggccgtgctggcggaacagcatggacctggcccgggaatgagcttcgtgaccacccaacctgaggcgcttgccgcggcggcaggccctggacccgggatgcatgtgtcttttgtgatggcctacccggaaatgctggccgcgggtccgggccccggagcctacggatcattcgtgcgcactgtgtccctccccgtgggagccggacctggaccggggttggagaatgataaccagctgctctacaactatccgggggccctcctcatcccaatcgccgtgggcggagccctggccggactggtgctcattgtcctgatcgcctaccttgtggggagaaaacggtcccatgcgggatatcaaacgatctgatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 37 - protein, 1123 aa, synthetic construct
MRVTAPRTLILLLSGALALTETWAAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGAQIYQAVSAQAAAIHGPGPGPSPSMGRDIKVQFQSGPGPGGINTIPIAINEAEYVGPGPGAAFQGAHARFVAAAAGPGPGAGWLAFFRDLVARGLGPGPGASIIRLVGAVLAEQHGPGPGMSFVTTQPEALAAAAGPGPGMHVSFVMAYPEMLAAGPGPGAYGSFVRTVSLPVGAGPGPGLENDNQLLYNYPGALGIVAGLAVLAVVVIGAVVAAVMCRRKSSGGKGGSYSQAACSDSAQGSDVSLTA

SEQ ID NO: 38 - RNA, 3565 nt, synthetic construct
  source 1..3565: mol_type = other RNA
acatttgcttctgacacaactgtgttcactagcaacctcaaacagccaccatgcgggtcactgcccccagaaccttgattttactactgtcgggagccctcgctctgactgaaacatgggccgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggacctgggcccggcgtggacttcggagccctgccacctgaaatcaactccgcccggatgtacgcaggacctggatccgccagcctggtggcggccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcggccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcgggccggggcctggatcacaaatcatgtacaactacccggcaatgctgggccacgctggagacatggccggctacgccggcacgctgcagtcactcggtgccgagatcgccgtcgagcaggcggcactgcagagcgcttggcagggagacactggcattacctaccaagcgtggcaggctcagtggaatcaagctatggaagatctggtccgcgcgtaccacgccatgtcctccactcacgaagccaacaccatggctatgatggccagggacactgccgaggcagccaaatgggggggaggaccggggccgggaaccataaactaccagtttggcgacgtcgacgcccacggagccatgatcagggcgcaggccgggtcgctggaagcagaacaccaggccatcatctccgatgtgctgaccgcctccgacttttggggaggagccggttcggctgcctgccaagggttcatcacacaattgggaaggaacttccaggtcatctacgagcaggccaatgcacacggtcaaaaggtccaagcggcgggcaacaacatggcccagactgactcggccgtgggcagcagttgggccggtcccggccctggccctgtcggggggcagtcatccttctactccgactggtactcccccgcctgcggaaaggccggctgtcagacctataagtgggaaaccttcctgacctccgaactcccgcagtggctctcggcgaatcgggcagtgaagcccaccgggtcggcggctattggactgtccatggccggttccagcgcgatgatcctggccgcctatcacccccagcaattcatctacgctggctccctgtccgccctgcttgacccatcgcaggggatgggtggaccgggccctggggcccagatctaccaagcagtgtcggcccaagccgcagcgatccatggaccaggaccgggaccgtccccgagcatgggccgggatattaaggttcagttccagtccggacccggcccggggggtattaacaccattccgattgccattaatgaggcagaatacgtcggccctggtcccggcgccgcgttccaaggagcccacgcacgattcgtggcggcagctgcgggacccgggcctggtgccggttggctggcttttttccgcgacctggtggccagaggacttggacccggtccgggcgccagcattatcaggctggtcggggccgtgctggcggaacagcatggacctggcccgggaatgagcttcgtgaccacccaacctgaggcgcttgccgcggcggcaggccctggacccgggatgcatgtgtcttttgtgatggcctacccggaaatgctggccgcgggtccgggccccggagcctacggatcattcgtgcgcactgtgtccctccccgtgggagccggacctggaccggggttggagaatgataaccagctgctctacaactatccgggggccctcggcatcgtggcgggcctcgctgtcctcgccgtggtggtgatcggagccgtcgtcgccgctgtgatgtgccgccgcaagtccagcggcggaaaggggggcagctatagccaggcagcctgttccgactccgcgcaggggtcggatgtgtccttgaccgcctgatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 39 - protein, 1109 aa, synthetic construct
MAAPGSARRPLLLLLLLLLLGLMHCASAAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGAQIYQAVSAQAAAIHGPGPGPSPSMGRDIKVQFQSGPGPGGINTIPIAINEAEYVGPGPGAAFQGAHARFVAAAAGPGPGAGWLAFFRDLVARGLGPGPGASIIRLVGAVLAEQHGPGPGMSFVTTQPEALAAAAGPGPGMHVSFVMAYPEMLAAGPGPGAYGSFVRTVSLPVGAGPGPGLENDNQLLYNYPGALLIPIAVGGALAGLVLIVLIAYLVGRKRSHAGYQTI

SEQ ID NO: 40 - RNA, 3523 nt, synthetic construct
  source 1..3523: mol_type = other RNA
acatttgcttctgacacaactgtgttcactagcaacctcaaacagccaccatggccgctcctggatccgcccgcagaccgttgttgctcctgttactactgctcttacttggactcatgcactgcgcctccgccgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggacctgggcccggcgtggacttcggagccctgccacctgaaatcaactccgcccggatgtacgcaggacctggatccgccagcctggtggcggccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcggccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcgggccggggcctggatcacaaatcatgtacaactacccggcaatgctgggccacgctggagacatggccggctacgccggcacgctgcagtcactcggtgccgagatcgccgtcgagcaggcggcactgcagagcgcttggcagggagacactggcattacctaccaagcgtggcaggctcagtggaatcaagctatggaagatctggtccgcgcgtaccacgccatgtcctccactcacgaagccaacaccatggctatgatggccagggacactgccgaggcagccaaatgggggggaggaccggggccgggaaccataaactaccagtttggcgacgtcgacgcccacggagccatgatcagggcgcaggccgggtcgctggaagcagaacaccaggccatcatctccgatgtgctgaccgcctccgacttttggggaggagccggttcggctgcctgccaagggttcatcacacaattgggaaggaacttccaggtcatctacgagcaggccaatgcacacggtcaaaaggtccaagcggcgggcaacaacatggcccagactgactcggccgtgggcagcagttgggccggtcccggccctggccctgtcggggggcagtcatccttctactccgactggtactcccccgcctgcggaaaggccggctgtcagacctataagtgggaaaccttcctgacctccgaactcccgcagtggctctcggcgaatcgggcagtgaagcccaccgggtcggcggctattggactgtccatggccggttccagcgcgatgatcctggccgcctatcacccccagcaattcatctacgctggctccctgtccgccctgcttgacccatcgcaggggatgggtggaccgggccctggggcccagatctaccaagcagtgtcggcccaagccgcagcgatccatggaccaggaccgggaccgtccccgagcatgggccgggatattaaggttcagttccagtccggacccggcccggggggtattaacaccattccgattgccattaatgaggcagaatacgtcggccctggtcccggcgccgcgttccaaggagcccacgcacgattcgtggcggcagctgcgggacccgggcctggtgccggttggctggcttttttccgcgacctggtggccagaggacttggacccggtccgggcgccagcattatcaggctggtcggggccgtgctggcggaacagcatggacctggcccgggaatgagcttcgtgaccacccaacctgaggcgcttgccgcggcggcaggccctggacccgggatgcatgtgtcttttgtgatggcctacccggaaatgctggccgcgggtccgggccccggagcctacggatcattcgtgcgcactgtgtccctccccgtgggagccggacctggaccggggttggagaatgataaccagctgctctacaactatccgggggccctcctgattcctatcgccgtgggcggtgccttggccggcctggtgctgatcgtcctgattgcctatctcgtggggaggaagagatcgcatgccggctaccagaccatctgatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 41 - protein, 1071 aa, synthetic construct
MAISGVPVLGFFIIAVLMSAQESWAAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGAQIYQAVSAQAAAIHGPGPGPSPSMGRDIKVQFQSGPGPGGINTIPIAINEAEYVGPGPGAAFQGAHARFVAAAAGPGPGAGWLAFFRDLVARGLGPGPGASIIRLVGAVLAEQHGPGPGMSFVTTQPEALAAAAGPGPGMHVSFVMAYPEMLAAGPGPGAYGSFVRTVSLPVGAGPGPGLENDNQLLYNYPGAL

SEQ ID NO: 42 - RNA, 3409 nt, synthetic construct
  source 1..3409: mol_type = other RNA
acatttgcttctgacacaactgtgttcactagcaacctcaaacagccaccatggccatctccggagtgcctgtgctcggttttttcatcatcgccgtgcttatgtccgcccaggaatcatgggccgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggacctgggcccggcgtggacttcggagccctgccacctgaaatcaactccgcccggatgtacgcaggacctggatccgccagcctggtggcggccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcggccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcgggccggggcctggatcacaaatcatgtacaactacccggcaatgctgggccacgctggagacatggccggctacgccggcacgctgcagtcactcggtgccgagatcgccgtcgagcaggcggcactgcagagcgcttggcagggagacactggcattacctaccaagcgtggcaggctcagtggaatcaagctatggaagatctggtccgcgcgtaccacgccatgtcctccactcacgaagccaacaccatggctatgatggccagggacactgccgaggcagccaaatgggggggaggaccggggccgggaaccataaactaccagtttggcgacgtcgacgcccacggagccatgatcagggcgcaggccgggtcgctggaagcagaacaccaggccatcatctccgatgtgctgaccgcctccgacttttggggaggagccggttcggctgcctgccaagggttcatcacacaattgggaaggaacttccaggtcatctacgagcaggccaatgcacacggtcaaaaggtccaagcggcgggcaacaacatggcccagactgactcggccgtgggcagcagttgggccggtcccggccctggccctgtcggggggcagtcatccttctactccgactggtactcccccgcctgcggaaaggccggctgtcagacctataagtgggaaaccttcctgacctccgaactcccgcagtggctctcggcgaatcgggcagtgaagcccaccgggtcggcggctattggactgtccatggccggttccagcgcgatgatcctggccgcctatcacccccagcaattcatctacgctggctccctgtccgccctgcttgacccatcgcaggggatgggtggaccgggccctggggcccagatctaccaagcagtgtcggcccaagccgcagcgatccatggaccaggaccgggaccgtccccgagcatgggccgggatattaaggttcagttccagtccggacccggcccggggggtattaacaccattccgattgccattaatgaggcagaatacgtcggccctggtcccggcgccgcgttccaaggagcccacgcacgattcgtggcggcagctgcgggacccgggcctggtgccggttggctggcttttttccgcgacctggtggccagaggacttggacccggtccgggcgccagcattatcaggctggtcggggccgtgctggcggaacagcatggacctggcccgggaatgagcttcgtgaccacccaacctgaggcgcttgccgcggcggcaggccctggacccgggatgcatgtgtcttttgtgatggcctacccggaaatgctggccgcgggtccgggccccggagcctacggatcattcgtgcgcactgtgtccctccccgtgggagccggacctggaccggggttggagaatgataaccagctgctctacaactatccgggggccctctgatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 43 - protein, 1068 aa, synthetic construct
MDAMKRGLCCVLLLCGAVFVSAAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGAQIYQAVSAQAAAIHGPGPGPSPSMGRDIKVQFQSGPGPGGINTIPIAINEAEYVGPGPGAAFQGAHARFVAAAAGPGPGAGWLAFFRDLVARGLGPGPGASIIRLVGAVLAEQHGPGPGMSFVTTQPEALAAAAGPGPGMHVSFVMAYPEMLAAGPGPGAYGSFVRTVSLPVGAGPGPGLENDNQLLYNYPGAL

SEQ ID NO: 44 - RNA, 3400 nt, synthetic construct
  source 1..3400: mol_type = other RNA
acatttgcttctgacacaactgtgttcactagcaacctcaaacagccaccatggatgctatgaagagaggattgtgttgcgtgttactactgtgcggagccgtgtttgtgtccgccgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggacctgggcccggcgtggacttcggagccctgccacctgaaatcaactccgcccggatgtacgcaggacctggatccgccagcctggtggcggccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcggccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcgggccggggcctggatcacaaatcatgtacaactacccggcaatgctgggccacgctggagacatggccggctacgccggcacgctgcagtcactcggtgccgagatcgccgtcgagcaggcggcactgcagagcgcttggcagggagacactggcattacctaccaagcgtggcaggctcagtggaatcaagctatggaagatctggtccgcgcgtaccacgccatgtcctccactcacgaagccaacaccatggctatgatggccagggacactgccgaggcagccaaatgggggggaggaccggggccgggaaccataaactaccagtttggcgacgtcgacgcccacggagccatgatcagggcgcaggccgggtcgctggaagcagaacaccaggccatcatctccgatgtgctgaccgcctccgacttttggggaggagccggttcggctgcctgccaagggttcatcacacaattgggaaggaacttccaggtcatctacgagcaggccaatgcacacggtcaaaaggtccaagcggcgggcaacaacatggcccagactgactcggccgtgggcagcagttgggccggtcccggccctggccctgtcggggggcagtcatccttctactccgactggtactcccccgcctgcggaaaggccggctgtcagacctataagtgggaaaccttcctgacctccgaactcccgcagtggctctcggcgaatcgggcagtgaagcccaccgggtcggcggctattggactgtccatggccggttccagcgcgatgatcctggccgcctatcacccccagcaattcatctacgctggctccctgtccgccctgcttgacccatcgcaggggatgggtggaccgggccctggggcccagatctaccaagcagtgtcggcccaagccgcagcgatccatggaccaggaccgggaccgtccccgagcatgggccgggatattaaggttcagttccagtccggacccggcccggggggtattaacaccattccgattgccattaatgaggcagaatacgtcggccctggtcccggcgccgcgttccaaggagcccacgcacgattcgtggcggcagctgcgggacccgggcctggtgccggttggctggcttttttccgcgacctggtggccagaggacttggacccggtccgggcgccagcattatcaggctggtcggggccgtgctggcggaacagcatggacctggcccgggaatgagcttcgtgaccacccaacctgaggcgcttgccgcggcggcaggccctggacccgggatgcatgtgtcttttgtgatggcctacccggaaatgctggccgcgggtccgggccccggagcctacggatcattcgtgcgcactgtgtccctccccgtgggagccggacctggaccggggttggagaatgataaccagctgctctacaactatccgggggccctctgatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 45 - protein, 205 aa, Mycobacterium tuberculosis
APKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLA

SEQ ID NO: 46 - protein, 65 aa, Mycobacterium tuberculosis
QAETAVNTLFEKLEPMASILDPGASQSTTNPIFGMPSPGSSTPVGQLPPAATQTLGQLGEMSGPM

SEQ ID NO: 47 - protein, 64 aa, Mycobacterium tuberculosis
GNPADEEAAQMGLLGTSPLSNHPLAGGSGPSAGAGLLRAESLPGAGGSLTRTPLMSQLIEKPVA

SEQ ID NO: 48 - protein, 666 aa, Mycobacterium tuberculosis
MAADYDKLFRPHEGMEAPDDMAAQPFFDPSASFPPAPASANLPKPNGQTPPPTSDDLSERFVSAPPPPPPPPPPPPPTPMPIAAGEPPSPEPAASKPPTPPMPIAGPEPAPPKPPTPPMPIAGPEPAPPKPPTPPMPIAGPAPTPTESQLAPPRPPTPQTPTGAPQQPESPAPHVPSHGPHQPRRTAPAPPWAKMPIGEPPPAPSRPSASPAEPPTRPAPQHSRRARRGHRYRTDTERNVGKVATGPSIQARLRAEEASGAQLAPGTEPSPAPLGQPRSYLAPPTRPAPTEPPPSPSPQRNSGRRAERRVHPDLAAQHAAAQPDSITAATTGGRRRKRAAPDLDATQKSLRPAAKGPKVKKVKPQKPKATKPPKVVSQRGWRHWVHALTRINLGLSPDEKYELDLHARVRRNPRGSYQIAVVGLKGGAGKTTLTAALGSTLAQVRADRILALDADPGAGNLADRVGRQSGATIADVLAEKELSHYNDIRAHTSVNAVNLEVLPAPEYSSAQRALSDADWHFIADPASRFYNLVLADCGAGFFDPLTRGVLSTVSGVVVVASVSIDGAQQASVALDWLRNNGYQDLASRACVVINHIMPGEPNVAVKDLVRHFEQQVQPGRVVVMPWDRHIAAGTEISLDLLDPIYKRKVLELAAALSDDFERAGRR

SEQ ID NO: 49 - protein, 103 aa, Mycobacterium tuberculosis
MTENLTVQPERLGVLASHHDNAAVDASSGVEAAAGLGESVAITHGPYCSQFNDTLNVYLTAHNALGSSLHTAGVDLAKSLRIAAKIYSEADEAWRKAIDGLFT

SEQ ID NO: 50 - protein, 392 aa, Mycobacterium tuberculosis
MSRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVV

SEQ ID NO: 51 - protein, 163 aa, Mycobacterium tuberculosis
GDLVGPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPA

SEQ ID NO: 52 - protein, 190 aa, Mycobacterium tuberculosis
VSQDTSPKPATSPAAPVTTAAMADPAADLIGRGCAQYAAQNPTGPGSVAGMAQDPVATAASNNPMLSTLTSALSGKLNPDVNLVDTLNGGEYTVFAPTNAAFDKLPAATIDQLKTDAKLLSSILTYHVIAGQASPSRIDGTHQTLQGADLTVIGARDDLMVNNAGLVCGGVHTANATVYMIDTVLMPPAQ

SEQ ID NO: 53 - protein, 184 aa, Mycobacterium tuberculosis
VDLPGNDFDSNDFDAVDLWGADGAEGWTADPIIGVGSAATPDTGPDLDNAHGQAETDTEQEIALFTVTNPPRTVSVSTLMDGRIDHVELSARVAWMSESQLASEILVIADLARQKAQSAQYAFILDRMSQQVDADEHRVALLRKTVGETWGLPSPEEAAAAEAEVFATRYSDDCPAPDDESDPW

SEQ ID NO: 54 - protein, 460 aa, Mycobacterium tuberculosis
MTQSQTVTVDQQEILNRANEVEAPMADPPTDVPITPCELTAAKNAAQQLVLSADNMREYLAAGAKERQRLATSLRNAAKAYGEVDEEAATALDNDGEGTVQAESAGAVGGDSSAELTDTPRVATAGEPNFMDLKEAARKLETGDQGASLAHFADGWNTFNLTLQGDVKRFRGFDNWEGDAATACEASLDQQRQWILHMAKLSAAMAKQAQYVAQLHVWARREHPTYEDIVGLERLYAENPSARDQILPVYAEYQQRSEKVLTEYNNKAALEPVNPPKPPPAIKIDPPPPPQEQGLIPGFLMPPSDGSGVTPGTGMPAAPMVPPTGSPGGGLPADTAAQLTSAGREAAALSGDVAVKAASLGGGGGGGVPSAPLGSAIGGAESVRPAGAGDIAGLGQGRAGGGAALGGGGMGMPMGAAHQGQGGAKSKGSQQEDEALYTEDRAWTEAVIGNRRRQDSKESK

SEQ ID NO: 55 - protein, 355 aa, Mycobacterium tuberculosis
MSNSRRRSLRWSWLLSVLAAVGLGLATAPAQAAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 56 - protein, 97 aa, Mycobacterium tuberculosis
MSLLDAHIPQLVASQSAFAAKAGLMRHTIGQAEQAAMSAQAFHQGESSAAFQAAHARFVAAAAKVNTLLDVAQANLGEAAGTYVAADAAAASTYTGF

SEQ ID NO: 57 - protein, 96 aa, Mycobacterium tuberculosis
MSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 58 - protein, 415 aa, Mycobacterium tuberculosis
MTSSHLIDAEQLLADQLAQASPDLLRGLLSTFIAALMGAEADALCGAGYRERSDERSNQRNGYRHRDFDTRAATIDVAIPKLRQGSYFPDWLLQRRKRAERALTSVVATCYLLGVSTRRMERLVETLGVTKLSKSQVSIMAKELDEAVEAFRTRPLDAGPYTFLAADALVLKVREAGRVVGVHTLIATGVNAEGYREILGIQVTSAEDGAGWLAFFRDLVARGLSGVALVTSDAHAGLVAAIGATLPAAAWQRCRTHYAANLMAATPKPSWPWVRTLLHSIYDQPDAESVVAQYDRVLDALTDKLPAVAEHLDTARTDLLAFTAFPKQIWRQIWSNNPQERLNREVRRRTDVVGIFPDRASIIRLVGAVLAEQHDEWIEGRRYLGLEVLTRARAALTSTEEPAKQQTTNTPALTT

SEQ ID NO: 59 - protein, 308 aa, Mycobacterium tuberculosis
MSFVFAAPEALAAAAADMAGIGSTLNAANVVAAVPTTGVLAAAADEVSTQVAALLSAHAQGYQQLSRQMMTAFHDQFVQALRASADAYATAEASAAQTMVNAVNAPARALLGHPLISADASTGGGSNALSRVQSMFLGTGGSSALGGSAAANAAASGALQLQPTGGASGLSAVGALLPRAGAAAAAALPALAAESIGNAIKNLYNAVEPWVQYGFNLTAWAVGWLPYIGILAPQINFFYYLGEPIVQAVLFNAIDFVDGTVTFSQALTNIETATAASINQFINTEINWIRGFLPPLPPISPPGFPSLP

SEQ ID NO: 60 - protein, 99 aa, Mycobacterium tuberculosis
VSFVMAYPEMLAAAADTLQSIGATTVASNAAAAAPTTGVVPPAADEVSALTAAHFAAHAAMYQSVSARAAAIHDQFVATLASSASSYAATEVANAAAAS

SEQ ID NO: 61 - protein, 391 aa, Mycobacterium tuberculosis
MVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAG

SEQ ID NO: 62 - protein, 539 aa, Mycobacterium tuberculosis
MTEPWIAFPPEVHSAMLNYGAGVGPMLISATQNGELSAQYAEAASEVEELLGVVASEGWQGQAAEAFVAAYMPFLAWLIQASADCVEMAAQQHVVIEAYTAAVELMPTQVELAANQIKLAVLVATNFFGINTIPIAINEAEYVEMWVRAATTMATYSTVSRSALSAMPHTSPPPLILKSDELLPDTGEDSDEDGHNHGGHSHGGHARMIDNFFAEILRGVSAGRIVWDPVNGTLNGLDYDDYVYPGHAIWWLARGLEFFQDGEQFGELLFTNPTGAFQFLLYVVVVDLPTHIAQIATWLGQYPQLLSAALTGVIAHLGAITGLAGLSGLSAIPSAAIPAVVPELTPVAAAPPMLAVAGVGPAVAAPGMLPASAPAPAAAAGATAAGPTPPATGFGGFPPYLVGGGGPGIGFGSGQSAHAKAAASDSAAAESAAQASARAQARAARRGRSAAKARGHRDEFVTMDMGFDAAAPAPEHQPGARASDCGAGPIGFAGTVRKEAVVKAAGLTTLAGDDFGGGPTMPMMPGTWTHDQGVFDEHR

SEQ ID NO: 63 - protein, 99 aa, Mycobacterium tuberculosis
MSFVTTQPEALAAAAGSLQGIGSALNAQNAAAATPTTGVVPAAADEVSALTAAQFAAHAQIYQAVSAQAAAIHEMFVNTLQMSSGSYAATEAANAAAAG

SEQ ID NO: 64 - protein, 325 aa, Mycobacterium tuberculosis
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 65 - protein, 740 aa, Mycobacterium tuberculosis
VPEQHPPITETTTGAASNGCPVVGHMKYPVEGGGNQDWWPNRLNLKVLHQNPAVADPMGAAFDYAAEVATIDVDALTRDIEEVMTTSQPWWPADYGHYGPLFIRMAWHAAGTYRIHDGRGGAGGGMQRFAPLNSWPDNASLDKARRLLWPVKKKYGKKLSWADLIVFAGNCALESMGFKTFGFGFGRVDQWEPDEVYWGKEATWLGDERYSGKRDLENPLAAVQMGLIYVNPEGPNGNPDPMAAAVDIRETFRRMAMNDVETAALIVGGHTFGKTHGAGPADLVGPEPEAAPLEQMGLGWKSSYGTGTGKDAITSGIEVVWTNTPTKWDNSFLEILYGYEWELTKSPAGAWQYTAKDGAGAGTIPDPFGGPGRSPTMLATDLSLRVDPIYERITRRWLEHPEELADEFAKAWYKLIHRDMGPVARYLGPLVPKQTLLWQDPVPAVSHDLVGEAEIASLKSQIRASGLTVSQLVSTAWAAASSFRGSDKRGGANGGRIRLQPQVGWEVNDPDGDLRKVIRTLEEIQESFNSAAPGNIKVSFADLVVLGGCAAIEKAAKAAGHNITVPFTPGRTDASQEQTDVESFAVLEPKADGFRNYLGKGNPLPAEYMLLDKANLLTLSAPEMTVLVGGLRVLGANYKRLPLGVFTEASESLTNDFFVNLLDMGITWEPSPADDGTYQGKDGSGKVKWTGSRVDLVFGSNSELRALVEVYGADDAQPKFVQDFVAAWDKVMNLDRFDVR

SEQ ID NO: 66 - protein, 159 aa, Mycobacterium tuberculosis
MKLTTMIKTAVAVVAMAAIATFAAPVALAAYPITGKLGSELTMTDTVGQVVLGWKVSDLKSSTAVIPGYPVAGQVWEATATVNAIRGSVTPAVSQFNARTADGINYRVLWQAAGPDTISGATIPQGEQSTGKIYFDVTGPSPTIVAMNNGMEDLLIWEP

SEQ ID NO: 67 - protein, 228 aa, Mycobacterium tuberculosis
VRIKIFMLVTAVVLLCCSGVATAAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLA

SEQ ID NO: 68 - protein, 144 aa, Mycobacterium tuberculosis
MATTLPVQRHPRSLFPEFSELFAAFPSFAGLRPTFDTRLMRLEDEMKEGRYEVRAELPGVDPDKDVDIMVRDGQLTIKAERTEQKDFDGRSEFAYGSFVRTVSLPVGADEDDIKATYDKGILTVSVAVSEGKPTEKHIQIRSTN

SEQ ID NO: 69 - protein, 580 aa, Mycobacterium tuberculosis
MNFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLF

SEQ ID NO: 70 - protein, 382 aa, Mycobacterium tuberculosis
VDFGALPPEVNSARMYGGAGAADLLAAAAAWNGIAVEVSTAASSVGSVITRLSTEHWMGPASLSMAAAVQPYLVWLTCTAESSALAAAQAMASAAAFETAFALTVPPAEVVANRALLAELTATNILGQNVSAIAATEARYGEMWAQDASAMYGYAAASAVAARLNPLTRPSHITNPAGLAHQAAAVGQAGASAFARQVGLSHLISDVADAVLSFASPVMSAADTGLEAVRQFLNLDVPLFVESAFHGLGGVADFATAAIGNMTLLADAMGTVGGAAPGGGAAAAVAHAVAPAGVGGTALTADLGNASVVGRLSVPASWSTAAPATAAGAALDGTGWAVPEEDGPIAVMPPAPGMVVAANSVGADSGPRYGVKPIVMPKHGLF

SEQ ID NO: 71 - protein, 220 aa, Mycobacterium tuberculosis
MINVQAKPAAAASLAAIAIAFLAGCSSTKPVSQDTSPKPATSPAAPVTTAAMADPAADLIGRGCAQYAAQNPTGPGSVAGMAQDPVATAASNNPMLSTLTSALSGKLNPDVNLVDTLNGGEYTVFAPTNAAFDKLPAATIDQLKTDAKLLSSILTYHVIAGQASPSRIDGTHQTLQGADLTVIGARDDLMVNNAGLVCGGVHTANATVYMIDTVLMPPAQ

SEQ ID NO: 72 - protein, 193 aa, Mycobacterium tuberculosis
MKVKNTIAATSFAAAGLAALAVAVSPPAAAGDLVGPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPA

SEQ ID NO: 73 - protein, 97 aa, Mycobacterium tuberculosis
MSLLDAHIPQLIASHTAFAAKAGLMRHTIGQAEQQAMSAQAFHQGESAAAFQGAHARFVAAAAKVNTLLDIAQANLGEAAGTYVAADAAAASSYTGF

SEQ ID NO: 74 - protein, 367 aa, Mycobacterium tuberculosis
MKVLAAMSGGVDSSVAAARMVDAGHEVVGVHMALSTAPGTLRTGSRGCCSKEDAADARRVADVLGIPFYVWDFAEKFKEDVINDFVSSYARGETPNPCVRCNQQIKFAALSARAVALGFDTVATGHYARLSGGRLRRAVDRDKDQSYVLAVLTAQQLRHAAFPIGDTPKRQIRAEAARRGLAVANKPDSHDICFIPSGNTKAFLGERIGVRRGVVVDADGVVLASHDGVHGFTIGQRRGLGIAGPGPNGRPRYVTAIDADTATVHVGDVTDLDVQTLTGRAPVFTAGAAPSGPVDCVVQVRAHGETVSAVAELIGDALFVQLHAPLRGVARGQTLVLYRPDPAGDEVLGSATIAGASGLSTGGNPGA

SEQ ID NO: 75 - protein, 380 aa, Mycobacterium tuberculosis
MDFALLPPEVNSARMYTGPGAGSLLAAAGGWDSLAAELATTAEAYGSVLSGLAALHWRGPAAESMAVTAAPYIGWLYTTAEKTQQTAIQARAAALAFEQAYAMTLPPPVVAANRIQLLALIATNFFGQNTAAIAATEAQYAEMWAQDAAAMYGYATASAAAALLTPFSPPRQTTNPAGLTAQAAAVSQATDPLSLLIETVTQALQALTIPSFIPEDFTFLDAIFAGYATVGVTQDVESFVAGTIGAESNLGLLNVGDENPAEVTPGDFGIGELVSATSPGGGVSASGAGGAASVGNTVLASVGRANSIGQLSVPPSWAAPSTRPVSALSPAGLTTLPGTDVAEHGMPGVPGVPVAAGRASGVLPRYGVRLTVMAHPPAAG

SEQ ID NO: 76 - protein, 405 aa, Mycobacterium tuberculosis
MAFLRSVSCLAAAVFAVGTGIGLPTAAGEPNAAPAACPYKVSTPPAVDSSEVPAAGEPPLPLVVPPTPVGGNALGGCGIITAPGSAPAPGDVSAEAWLVADLDSGAVIAARDPHGRHRPASVIKVLVAMASINTLTLNKSVAGTADDAAVEGTKVGVNTGGTYTVNQLLHGLLMHSGNDAAYALARQLGGMPAALEKINLLAAKLGGRDTRVATPSGLDGPGMSTSAYDIGLFYRYAWQNPVFADIVATRTFDFPGHGDHPGYELENDNQLLYNYPGALGGKTGYTDDAGQTFVGAANRDGRRLMTVLLHGTRQPIPPWEQAAHLLDYGFNTPAGTQIGTLIEPDPSLMSTDRNPADRQRVDPQAAARISAADALPVRVGVAVIGALIVFGLIMVARAMNRRPQH

SEQ ID NO: 77 - protein, 103 aa, Mycobacterium tuberculosis; identical to SEQ ID NO: 49

SEQ ID NO: 78 - protein, 392 aa, Mycobacterium tuberculosis; identical to SEQ ID NO: 50

SEQ ID NO: 79 - protein, 94 aa, Mycobacterium tuberculosis
MTINYQFGDVDAHGAMIRAQAGSLEAEHQAIISDVLTASDFWGGAGSAACQGFITQLGRNFQVIYEQANAHGQKVQAAGNNMAQTDSAVGSSWA

SEQ ID NO: 80 - protein, 98 aa, Mycobacterium tuberculosis
MTSRFMTDPHAMRDMAGRFEVHAQTVEDEARRMWASAQNISGAGWSGMAEATSLDTMTQMNQAFRNIVNMLHGVRDGLVRDANNYEQQEQASQQILSS

SEQ ID NO: 81 - protein, 338 aa, Mycobacterium tuberculosis
MQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGA

SEQ ID NO: 82 - protein, 368 aa, Mycobacterium tuberculosis
MLWHAMPPELNTARLMAGAGPAPMLAAAAGWQTLSAALDAQAVELTARLNSLGEAWTGGGSDKALAAATPMVVWLQTASTQAKTRAMQATAQAAAYTQAMATTPSLPEIAANHITQAVLTATNFFGINTIPIALTEMDYFIRMWNQAALAMEVYQAETAVNTLFEKLEPMASILDPGASQSTTNPIFGMPSPGSSTPVGQLPPAATQTLGQLGEMSGPMQQLTQPLQQVTSLFSQVGGTGGGNPADEEAAQMGLLGTSPLSNHPLAGGSGPSAGAGLLRAESLPGAGGSLTRTPLMSQLIEKPVAPSVMPAAAAGSSATGGAAPVGAGAMGQGAQSGGSTRPGLVAPAPLAQEREEDDEDDWDEEDDW

SEQ ID NO: 83 - protein, 100 aa, Mycobacterium tuberculosis
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 84 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 85 - protein, 666 aa, Mycobacterium tuberculosis; identical to SEQ ID NO: 48

SEQ ID NO: 86 - protein, 1061 aa, synthetic construct
FVRPVAVDLTYIPVVGHALSARLYAENPSARDQILPVYAEYQQRSEKVLTEYNNKAALDQQRQWILHMAKLSAAMAKQAQYVAQLHVWARREHPTYERRTAPAPPWAKMWRHWVHALTRINLGLSPDEKYELDLHARVRRNPRGSYQIAVVGLPAAPVTTAAMADPAADLIAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAAAKNAAQQLVLSADNMREYLAAGAKERQRLATSLRNAAKMAVVGGALAYLVVKTLINATQLLKLLAKLQLPPAATQTLAEAEVFATRYSGVSTANATVYMIDSVLMAETDTEQEIALFTVTNPPRTVSVSTLMDGRIDHVELSARVAWMSESQLASEILVIADLARQKAQSAQYAFILDRMSQQVDADEHRVALLFIIDPTISAIDGLYDLLGIGIPNQGGILYSSLEYFEKALEELAVHAASTRQALRSQFNDTLNVYLTAHNALGSSLHTAGVDLAKFFDPLTRGVLAEQVGGQSQLGVHTANATVYMIDTVLMATIADVLAEKELSHYNDIRAHTSVNAVNLEVLPAPEYSSAQRALSDADWHFIADPASRFYNLVLAQPFFDPSASFPGKLNPDVNLVDTLNGGEYTVFAPTNAAFDKLPAATIDQLKTDAKLLSSILTYHVIAGQASPSRIDGTHQTLNHIMPGEPNVAVKDLVRHFEQQVQPGRVVVMPWDRHIAAGTEISLDLLDPIYKRKVLELAAALQAETAVNTLFEKLEPMAAASKPPTPPMASKGTTTKKYMAADYDKLFRAHFADGWNTFNLTRTPLMSQLIGWTADPIIGVQVRADRILALAVAASNNPELTTLTAALSGQLNPQVNLVDTLNSGQYTVFAPTNAAFSKLPASTIDELKTNSSLLTSILTY

SEQ ID NO: 87 - protein, 1061 aa, synthetic construct
MAADYDKLFRAHFADGWNTFNLSQFNDTLNVYLTAHNALGSSLHTAGVDLAKAVAASNNPELTTLTAALSGQLNPQVNLVDTLNSGQYTVFAPTNAAFSKLPASTIDELKTNSSLLTSILTYNHIMPGEPNVAVKDLVRHFEQQVQPGRVVVMPWDRHIAAGTEISLDLLDPIYKRKVLELAAALFFDPLTRGVLRRTAPAPPWAKMWRHWVHALTRINLGLSPDEKYELDLHARVRRNPRGSYQIAVVGLPAAPVTTAAMADPAADLIAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAGKLNPDVNLVDTLNGGEYTVFAPTNAAFDKLPAATIDQLKTDAKLLSSILTYHVIAGQASPSRIDGTHQTLAEQVGGQSQLGVSTANATVYMIDSVLMAETDTEQEIALFTVTNPPRTVSVSTLMDGRIDHVELSARVAWMSESQLASEILVIADLARQKAQSAQYAFILDRMSQQVDADEHRVALLGWTADPIIGVQVRADRILALRLYAENPSARDQILPVYAEYQQRSEKVLTEYNNKAALDQQRQWILHMAKLSAAMAKQAQYVAQLHVWARREHPTYEAAKNAAQQLVLSADNMREYLAAGAKERQRLATSLRNAAKATIADVLAEKELSHYNDIRAHTSVNAVNLEVLPAPEYSSAQRALSDADWHFIADPASRFYNLVLAFVRPVAVDLTYIPVVGHALSAASKGTTTKKYQLPPAATQTLTRTPLMSQLIFIIDPTISAIDGLYDLLGIGIPNQGGILYSSLEYFEKALEELAQPFFDPSASFPMAVVGGALAYLVVKTLINATQLLKLLAKLQAETAVNTLFEKLEPMAAASKPPTPPMVHAASTRQALRGVHTANATVYMIDTVLMAEAEVFATRYS

SEQ ID NO: 88 - protein, 1061 aa, synthetic construct
WRHWVHALTRINLGLSPDEKYELDLHARVRRNPRGSYQIAVVGLMAADYDKLFRAHFADGWNTFNLSQFNDTLNVYLTAHNALGSSLHTAGVDLAKFIIDPTISAIDGLYDLLGIGIPNQGGILYSSLEYFEKALEELAAEQVGGQSQLGVSTANATVYMIDSVLMAETDTEQEIALFTVTNPPRTVSVSTLMDGRIDHVELSARVAWMSESQLASEILVIADLARQKAQSAQYAFILDRMSQQVDADEHRVALLGWTADPIIGVQVRADRILALRLYAENPSARDQILPVYAEYQQRSEKVLTEYNNKAALDQQRQWILHMAKLSAAMAKQAQYVAQLHVWARREHPTYEAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAAAKNAAQQLVLSADNMREYLAAGAKERQRLATSLRNAAKATIADVLAEKELSHYNDIRAHTSVNAVNLEVLPAPEYSSAQRALSDADWHFIADPASRFYNLVLAQPFFDPSASFPMAVVGGALAYLVVKTLINATQLLKLLAKLQLPPAATQTLFVRPVAVDLTYIPVVGHALSAQAETAVNTLFEKLEPMAAASKPPTPPMVHAASTRQALRASKGTTTKKYPAAPVTTAAMADPAADLIGVHTANATVYMIDTVLMAEAEVFATRYSGKLNPDVNLVDTLNGGEYTVFAPTNAAFDKLPAATIDQLKTDAKLLSSILTYHVIAGQASPSRIDGTHQTLAVAASNNPELTTLTAALSGQLNPQVNLVDTLNSGQYTVFAPTNAAFSKLPASTIDELKTNSSLLTSILTYNHIMPGEPNVAVKDLVRHFEQQVQPGRVVVMPWDRHIAAGTEISLDLLDPIYKRKVLELAAALFFDPLTRGVLRRTAPAPPWAKMTRTPLMSQLI

SEQ ID NO: 89 - protein, 1061 aa, synthetic construct
FFDPLTRGVLTRTPLMSQLIGWTADPIIGVQVRADRILALRLYAENPSARDQILPVYAEYQQRSEKVLTEYNNKAALDQQRQWILHMAKLSAAMAKQAQYVAQLHVWARREHPTYEAAKNAAQQLVLSADNMREYLAAGAKERQRLATSLRNAAKATIADVLAEKELSHYNDIRAHTSVNAVNLEVLPAPEYSSAQRALSDADWHFIADPASRFYNLVLAAHFADGWNTFNLMAADYDKLFRSQFNDTLNVYLTAHNALGSSLHTAGVDLAKAVAASNNPELTTLTAALSGQLNPQVNLVDTLNSGQYTVFAPTNAAFSKLPASTIDELKTNSSLLTSILTYNHIMPGEPNVAVKDLVRHFEQQVQPGRVVVMPWDRHIAAGTEISLDLLDPIYKRKVLELAAALRRTAPAPPWAKMWRHWVHALTRINLGLSPDEKYELDLHARVRRNPRGSYQIAVVGLPAAPVTTAAMADPAADLIAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAGKLNPDVNLVDTLNGGEYTVFAPTNAAFDKLPAATIDQLKTDAKLLSSILTYHVIAGQASPSRIDGTHQTLAEQVGGQSQLGVSTANATVYMIDSVLMQPFFDPSASFPAETDTEQEIALFTVTNPPRTVSVSTLMDGRIDHVELSARVAWMSESQLASEILVIADLARQKAQSAQYAFILDRMSQQVDADEHRVALLFIIDPTISAIDGLYDLLGIGIPNQGGILYSSLEYFEKALEELAASKGTTTKKYQLPPAATQTLFVRPVAVDLTYIPVVGHALSAMAVVGGALAYLVVKTLINATQLLKLLAKLQAETAVNTLFEKLEPMAAASKPPTPPMVHAASTRQALRGVHTANATVYMIDTVLMAEAEVFATRYS

SEQ ID NO: 90 - protein, 1061 aa, synthetic construct
FFDPLTRGVLTRTPLMSQLIAEQVGGQSQLGVSTANATVYMIDSVLMAETDTEQEIALFTVTNPPRTVSVSTLMDGRIDHVELSARVAWMSESQLASEILVIADLARQKAQSAQYAFILDRMSQQVDADEHRVALLGWTADPIIGVQVRADRILALRLYAENPSARDQILPVYAEYQQRSEKVLTEYNNKAALDQQRQWILHMAKLSAAMAKQAQYVAQLHVWARREHPTYEAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAAAKNAAQQLVLSADNMREYLAAGAKERQRLATSLRNAAKAHFADGWNTFNLSQFNDTLNVYLTAHNALGSSLHTAGVDLAKFIIDPTISAIDGLYDLLGIGIPNQGGILYSSLEYFEKALEELAATIADVLAEKELSHYNDIRAHTSVNAVNLEVLPAPEYSSAQRALSDADWHFIADPASRFYNLVLAMAVVGGALAYLVVKTLINATQLLKLLAKLQLPPAATQTLFVRPVAVDLTYIPVVGHALSAQAETAVNTLFEKLEPMAAASKPPTPPMVHAASTRQALRASKGTTTKKYPAAPVTTAAMADPAADLIGVHTANATVYMIDTVLMAEAEVFATRYSGKLNPDVNLVDTLNGGEYTVFAPTNAAFDKLPAATIDQLKTDAKLLSSILTYHVIAGQASPSRIDGTHQTLAVAASNNPELTTLTAALSGQLNPQVNLVDTLNSGQYTVFAPTNAAFSKLPASTIDELKTNSSLLTSILTYNHIMPGEPNVAVKDLVRHFEQQVQPGRVVVMPWDRHIAAGTEISLDLLDPIYKRKVLELAAALRRTAPAPPWAKMWRHWVHALTRINLGLSPDEKYELDLHARVRRNPRGSYQIAVVGLMAADYDKLFRQPFFDPSASFP

SEQ ID NO: 91 - protein, 1061 aa, synthetic construct
WRHWVHALTRINLGLSPDEKYELDLHARVRRNPRGSYQIAVVGLQLPPAATQTLPAAPVTTAAMADPAADLIAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAAETDTEQEIALFTVTNPPRTVSVSTLMDGRIDHVELSARVAWMSESQLASEILVIADLARQKAQSAQYAFILDRMSQQVDADEHRVALLGWTADPIIGVQVRADRILALAVAASNNPELTTLTAALSGQLNPQVNLVDTLNSGQYTVFAPTNAAFSKLPASTIDELKTNSSLLTSILTYNHIMPGEPNVAVKDLVRHFEQQVQPGRVVVMPWDRHIAAGTEISLDLLDPIYKRKVLELAAALQAETAVNTLFEKLEPMAAASKPPTPPMVHAASTRQALRAHFADGWNTFNLAEAEVFATRYSAAKNAAQQLVLSADNMREYLAAGAKERQRLATSLRNAAKRLYAENPSARDQILPVYAEYQQRSEKVLTEYNNKAALDQQRQWILHMAKLSAAMAKQAQYVAQLHVWARREHPTYEGVHTANATVYMIDTVLMASKGTTTKKYMAADYDKLFRATIADVLAEKELSHYNDIRAHTSVNAVNLEVLPAPEYSSAQRALSDADWHFIADPASRFYNLVLAFVRPVAVDLTYIPVVGHALSASQFNDTLNVYLTAHNALGSSLHTAGVDLAKFFDPLTRGVLTRTPLMSQLIFIIDPTISAIDGLYDLLGIGIPNQGGILYSSLEYFEKALEELAAEQVGGQSQLGKLNPDVNLVDTLNGGEYTVFAPTNAAFDKLPAATIDQLKTDAKLLSSILTYHVIAGQASPSRIDGTHQTLRRTAPAPPWAKMMAVVGGALAYLVVKTLINATQLLKLLAKLGVSTANATVYMIDSVLMQPFFDPSASFP

SEQ ID NO: 92 - protein, 1061 aa, synthetic construct
APKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAAAKNAAQQLVLSADNMREYLAAGAKERQRLATSLRNAAKASKGTTTKKYQLPPAATQTLFVRPVAVDLTYIPVVGHALSAAVAASNNPELTTLTAALSGQLNPQVNLVDTLNSGQYTVFAPTNAAFSKLPASTIDELKTNSSLLTSILTYNHIMPGEPNVAVKDLVRHFEQQVQPGRVVVMPWDRHIAAGTEISLDLLDPIYKRKVLELAAALFFDPLTRGVLTRTPLMSQLIGWTADPIIGVAASKPPTPPMRRTAPAPPWAKMWRHWVHALTRINLGLSPDEKYELDLHARVRRNPRGSYQIAVVGLQAETAVNTLFEKLEPMAQVRADRILALRLYAENPSARDQILPVYAEYQQRSEKVLTEYNNKAALDQQRQWILHMAKLSAAMAKQAQYVAQLHVWARREHPTYEGKLNPDVNLVDTLNGGEYTVFAPTNAAFDKLPAATIDQLKTDAKLLSSILTYHVIAGQASPSRIDGTHQTLAEQVGGQSQLPAAPVTTAAMADPAADLIMAADYDKLFRSQFNDTLNVYLTAHNALGSSLHTAGVDLAKFIIDPTISAIDGLYDLLGIGIPNQGGILYSSLEYFEKALEELAVHAASTRQALRGVHTANATVYMIDTVLMATIADVLAEKELSHYNDIRAHTSVNAVNLEVLPAPEYSSAQRALSDADWHFIADPASRFYNLVLAAHFADGWNTFNLAEAEVFATRYSAETDTEQEIALFTVTNPPRTVSVSTLMDGRIDHVELSARVAWMSESQLASEILVIADLARQKAQSAQYAFILDRMSQQVDADEHRVALLMAVVGGALAYLVVKTLINATQLLKLLAKLGVSTANATVYMIDSVLMQPFFDPSASFP

SEQ ID NO: 93 - protein, 1061 aa, synthetic construct
WRHWVHALTRINLGLSPDEKYELDLHARVRRNPRGSYQIAVVGLQAETAVNTLFEKLEPMAGWTADPIIGVQLPPAATQTLFVRPVAVDLTYIPVVGHALSATRTPLMSQLIFIIDPTISAIDGLYDLLGIGIPNQGGILYSSLEYFEKALEELAASKGTTTKKYAEAEVFATRYSGKLNPDVNLVDTLNGGEYTVFAPTNAAFDKLPAATIDQLKTDAKLLSSILTYHVIAGQASPSRIDGTHQTLGVHTANATVYMIDTVLMATIADVLAEKELSHYNDIRAHTSVNAVNLEVLPAPEYSSAQRALSDADWHFIADPASRFYNLVLAAHFADGWNTFNLPAAPVTTAAMADPAADLIAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAAETDTEQEIALFTVTNPPRTVSVSTLMDGRIDHVELSARVAWMSESQLASEILVIADLARQKAQSAQYAFILDRMSQQVDADEHRVALLSQFNDTLNVYLTAHNALGSSLHTAGVDLAKFFDPLTRGVLAVAASNNPELTTLTAALSGQLNPQVNLVDTLNSGQYTVFAPTNAAFSKLPASTIDELKTNSSLLTSILTYNHIMPGEPNVAVKDLVRHFEQQVQPGRVVVMPWDRHIAAGTEISLDLLDPIYKRKVLELAAALAASKPPTPPMRRTAPAPPWAKMMAADYDKLFRAEQVGGQSQLQVRADRILALRLYAENPSARDQILPVYAEYQQRSEKVLTEYNNKAALDQQRQWILHMAKLSAAMAKQAQYVAQLHVWARREHPTYEAAKNAAQQLVLSADNMREYLAAGAKERQRLATSLRNAAKVHAASTRQALRMAVVGGALAYLVVKTLINATQLLKLLAKLGVSTANATVYMIDSVLMQPFFDPSASFP

SEQ ID NO: 94 - protein, 1061 aa, synthetic construct
WRHWVHALTRINLGLSPDEKYELDLHARVRRNPRGSYQIAVVGLASKGTTTKKYPAAPVTTAAMADPAADLIMAADYDKLFRATIADVLAEKELSHYNDIRAHTSVNAVNLEVLPAPEYSSAQRALSDADWHFIADPASRFYNLVLAFVRPVAVDLTYIPVVGHALSAAVAASNNPELTTLTAALSGQLNPQVNLVDTLNSGQYTVFAPTNAAFSKLPASTIDELKTNSSLLTSILTYAETDTEQEIALFTVTNPPRTVSVSTLMDGRIDHVELSARVAWMSESQLASEILVIADLARQKAQSAQYAFILDRMSQQVDADEHRVALLQVRADRILALRLYAENPSARDQILPVYAEYQQRSEKVLTEYNNKAALDQQRQWILHMAKLSAAMAKQAQYVAQLHVWARREHPTYETRTPLMSQLIFIIDPTISAIDGLYDLLGIGIPNQGGILYSSLEYFEKALEELAVHAASTRQALRAEQVGGQSQLAHFADGWNTFNLSQFNDTLNVYLTAHNALGSSLHTAGVDLAKFFDPLTRGVLGWTADPIIGVQAETAVNTLFEKLEPMAAASKPPTPPMRRTAPAPPWAKMAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAAAKNAAQQLVLSADNMREYLAAGAKERQRLATSLRNAAKMAVVGGALAYLVVKTLINATQLLKLLAKLQLPPAATQTLGVHTANATVYMIDTVLMAEAEVFATRYSGKLNPDVNLVDTLNGGEYTVFAPTNAAFDKLPAATIDQLKTDAKLLSSILTYHVIAGQASPSRIDGTHQTLNHIMPGEPNVAVKDLVRHFEQQVQPGRVVVMPWDRHIAAGTEISLDLLDPIYKRKVLELAAALGVSTANATVYMIDSVLMQPFFDPSASFP

SEQ ID NO: 95 - protein, 1061 aa, synthetic construct
FFDPLTRGVLAASKPPTPPMWRHWVHALTRINLGLSPDEKYELDLHARVRRNPRGSYQIAVVGLQLPPAATQTLTRTPLMSQLIRRTAPAPPWAKMAAKNAAQQLVLSADNMREYLAAGAKERQRLATSLRNAAKQPFFDPSASFPMAVVGGALAYLVVKTLINATQLLKLLAKLQAETAVNTLFEKLEPMADQQRQWILHMAKLSAAMAKQAQYVAQLHVWARREHPTYEGVHTANATVYMIDTVLMAEAEVFATRYSGKLNPDVNLVDTLNGGEYTVFAPTNAAFDKLPAATIDQLKTDAKLLSSILTYHVIAGQASPSRIDGTHQTLAEQVGGQSQLPAAPVTTAAMADPAADLIMAADYDKLFRATIADVLAEKELSHYNDIRAHTSVNAVNLEVLPAPEYSSAQRALSDADWHFIADPASRFYNLVLAFVRPVAVDLTYIPVVGHALSARLYAENPSARDQILPVYAEYQQRSEKVLTEYNNKAALSQFNDTLNVYLTAHNALGSSLHTAGVDLAKFIIDPTISAIDGLYDLLGIGIPNQGGILYSSLEYFEKALEELAVHAASTRQALRAHFADGWNTFNLASKGTTTKKYAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAAETDTEQEIALFTVTNPPRTVSVSTLMDGRIDHVELSARVAWMSESQLASEILVIADLARQKAQSAQYAFILDRMSQQVDADEHRVALLGWTADPIIGVQVRADRILALAVAASNNPELTTLTAALSGQLNPQVNLVDTLNSGQYTVFAPTNAAFSKLPASTIDELKTNSSLLTSILTYNHIMPGEPNVAVKDLVRHFEQQVQPGRVVVMPWDRHIAAGTEISLDLLDPIYKRKVLELAAALGVSTANATVYMIDSVLM

SEQ ID NO: 96 - protein, 1047 aa, synthetic construct
FYVWDFAEKFKEDVINDFVSSYPHTSPPPLILKSWERNDPTQQISQATDPLSLLIETVTQALQALTIPSFIPEDFTFLSRIDGTHQTLVAELIGDALFVQLHAPLRGVARGQTLVLYIRMWNQAALAMEVYQAETAVNTLFVAAWDKVMNLTYIPVVGHALSMAVVGGALAYLVVKTLINATQLLKLLAKLGRASGVLPRYGLPPPWQPALRHPRSLFPEFSELFAAFPSFAGLRPTFDTRLMRSLLDAHIPQLVAFHDQFVQALRAAIPAVVPELTPVAAAPPMLAVAATYDKGILTVYYQSGLSIVMDTRVATPSGLDGPGMSTSAYDIGLFYRYAWQNPVFGGTHPTTTYKAFDWDQAYRKPITYIEKPVAPSVMILAAYHPQQFIYAGSLSALLELENDNQLLYIMYNYPAMLGVHAASTRQALRVEMAAQQHVVIEAYTAAVELMPTQVELAMVRDGQLTIKAKLLSSILTYHGSVTPAVSQFNARTADGINYRVTLAIYHPQQFVYSRFMTDPHAMRAAAVDIRETFRSELRALVEVYLLPLPNIPLLEYAARFITPVHPGYTATFLETPSQFFPFTGLNSLTAARALPLTSLTSAAERGPGQMLDYVYPGHAIWWLARGLEFFQDGEQFGELLFTNPTGAFDFPDSGTHSWEGFAIPIGQAMAGYTDDAGQTFVQELDEISTNIQAAEAFVAAYMPFLVSSPDVLTTYIAAADEVSTQVAKELDEAVEAFIDELKTNSSLLTSILTYIAVMPPAPGMVRAVPGRVVALFSRPGLPVEYLQVPSPSMGAAFQGAHARFAFPPEVHSAMLILAPQINFFYSAQAAAIHEMFAFQYDGVNDFPKYPLNVFATANAIAGILFLHSGLIALADTDPLPVVFATIDVAIPKLRSQFNDTLNVYVRAATTMATYPISPPGFPSLPMYAGPGSASLRVLDALTDKLATIATFEMRYLQSLPAHLRPGLDELYAMTLPPPVVAANRIQLLLLDAHIPQLIAVADPMGAAFDYAAEVLRVPPRPYVMPHSPAAGFAAPVALAAYPITGKL

SEQ ID NO: 97 - protein, 1047 aa, synthetic construct
FYVWDFAEKFKEDVINDFVSSYRVLDALTDKLATIATFEMRYLQSLPAHLRPGLDELLRVPPRPYVMPHSPAAGFAAPVALAAYPITGKLQELDEISTNIGRASGVLPRYAFHDQFVQALRQAAEAFVAAYMPFLGLPPPWQPALRHPRSLFPEFSELFAAFPSFAGLRPTFDTRLMRDYVYPGHAIWWLARGLEFFQDGEQFGELLFTNPTGAFLLDAHIPQLIAVADPMGAAFDYAAEVRAVPGRVVALATIDVAIPKLRSQFNDTLNVYDFPDSGTHSWEAFPPEVHSAMLIEKPVAPSVMDTRVATPSGLDGPGMSTSAYDIGLFYRYAWQNPVFAAADEVSTQVAAAAVDIRETFRGSVTPAVSQFNARTADGINYRVTYIPVVGHALSMAVVGGALAYLVVKTLINATQLLKLLAKLPISPPGFPSLPYAMTLPPPVVAANRIQLLVHAASTRQALRATYDKGILTVMYAGPGSASLVEMAAQQHVVIEAYTAAVELMPTQVELASLLDAHIPQLVILAPQINFFYILAAYHPQQFIYAGSLSALLLLPLPNIPLLEYAARFITPVHPGYTATFLETPSQFFPFTGLNSLTMVRDGQLTIKAKLLSSILTYHAAIPAVVPELTPVAAAPPMLAVASQATDPLSLLIETVTQALQALTIPSFIPEDFTFLGFAIPIGQAMAGYTDDAGQTFVSRFMTDPHAMRVAAWDKVMNLYYQSGLSIVMAARALPLTSLTSAAERGPGQMLIRMWNQAALAMEVYQAETAVNTLFVAELIGDALFVQLHAPLRGVARGQTLVLYTLAIYHPQQFVYIAVMPPAPGMVAAFQGAHARFVSSPDVLTTYIGGTHPTTTYKAFDWDQAYRKPITYIMYNYPAMLGFSRPGLPVEYLQVPSPSMGSELRALVEVYIDELKTNSSLLTSILTYPHTSPPPLILKSWERNDPTQQIVRAATTMATYELENDNQLLYSRIDGTHQTLSAQAAAIHEMFAFQYDGVNDFPKYPLNVFATANAIAGILFLHSGLIALADTDPLPVVFKELDEAVEAF

SEQ ID NO: 98 - protein, 1047 aa, synthetic construct
FYVWDFAEKFKEDVINDFVSSYAVADPMGAAFDYAAEVMYAGPGSASLQELDEISTNIVRAATTMATYVSSPDVLTTYIYAMTLPPPVVAANRIQLLSLLDAHIPQLVILAPQINFFYTLAIYHPQQFVYDTRVATPSGLDGPGMSTSAYDIGLFYRYAWQNPVFGGTHPTTTYKAFDWDQAYRKPITYIMYNYPAMLGADTDPLPVVFATYDKGILTVMVRDGQLTIKDYVYPGHAIWWLARGLEFFQDGEQFGELLFTNPTGAFVAELIGDALFVQLHAPLRGVARGQTLVLYVAAWDKVMNLTYIPVVGHALSIAVMPPAPGMVFSRPGLPVEYLQVPSPSMGSQFNDTLNVYELENDNQLLYSRIDGTHQTLGYTDDAGQTFVAAFQGAHARFAFPPEVHSAMLAAAVDIRETFRMAVVGGALAYLVVKTLINATQLLKLLAKLATIATFEMRYLQSLPAHLRPGLDELLRVPPRPYVMPHSPAAGRVLDALTDKLGLPPPWQPALRHPRSLFPEFSELFAAFPSFAGLRPTFDTRLMRAAIPAVVPELTPVAAAPPMLAVASQATDPLSLLIETVTQALQALTIPSFIPEDFTFLVHAASTRQALRVEMAAQQHVVIEAYTAAVELMPTQVELAAAADEVSTQVAKELDEAVEAFIDELKTNSSLLTSILTYAKLLSSILTYHGSVTPAVSQFNARTADGINYRVGFAIPIGQAMAYYQSGLSIVMSAQAAAIHEMFDFPDSGTHSWERAVPGRVVALATIDVAIPKLRSELRALVEVYPISPPGFPSLPWERNDPTQQIIEKPVAPSVMILAAYHPQQFIYAGSLSALLLLPLPNIPLLEYAARFITPVHPGYTATFLETPSQFFPFTGLNSLTAARALPLTSLTSAAERGPGQMLIRMWNQAALAMEVYQAETAVNTLFAFQYDGVNDFPKYPLNVFATANAIAGILFLHSGLIALSRFMTDPHAMRFAAPVALAAYPITGKLGRASGVLPRYLLDAHIPQLIPHTSPPPLILKSAFHDQFVQALRQAAEAFVAAYMPFL

SEQ ID NO: 99 - protein, 1047 aa, synthetic construct
FYVWDFAEKFKEDVINDFVSSYGSVTPAVSQFNARTADGINYRVTYIPVVGHALSIRMWNQAALAMEVYQAETAVNTLFVAAWDKVMNLAFPPEVHSAMLSQATDPLSLLIETVTQALQALTIPSFIPEDFTFLAAAVDIRETFRMAVVGGALAYLVVKTLINATQLLKLLAKLGRASGVLPRYGLPPPWQPALRHPRSLFPEFSELFAAFPSFAGLRPTFDTRLMRSLLDAHIPQLVQAAEAFVAAYMPFLADTDPLPVVFFAAPVALAAYPITGKLATIATFEMRYLQSLPAHLRPGLDELYAMTLPPPVVAANRIQLLQELDEISTNIVSSPDVLTTYIGGTHPTTTYKAFDWDQAYRKPITYIMYNYPAMLGFSRPGLPVEYLQVPSPSMGIEKPVAPSVMILAAYHPQQFIYAGSLSALLVAELIGDALFVQLHAPLRGVARGQTLVLYVEMAAQQHVVIEAYTAAVELMPTQVELAGYTDDAGQTFVSRFMTDPHAMRVRAATTMATYSAQAAAIHEMFAFQYDGVNDFPKYPLNVFATANAIAGILFLHSGLIALAAADEVSTQVAKELDEAVEAFLLPLPNIPLLEYAARFITPVHPGYTATFLETPSQFFPFTGLNSLTAARALPLTSLTSAAERGPGQMLDYVYPGHAIWWLARGLEFFQDGEQFGELLFTNPTGAFYYQSGLSIVMDTRVATPSGLDGPGMSTSAYDIGLFYRYAWQNPVFIDELKTNSSLLTSILTYAKLLSSILTYHSELRALVEVYPISPPGFPSLPDFPDSGTHSWEVHAASTRQALRILAPQINFFYTLAIYHPQQFVYIAVMPPAPGMVATYDKGILTVMYAGPGSASLAAFQGAHARFGFAIPIGQAMAELENDNQLLYLRVPPRPYVMPHSPAAGRVLDALTDKLAFHDQFVQALRAAIPAVVPELTPVAAAPPMLAVASRIDGTHQTLLLDAHIPQLIAVADPMGAAFDYAAEVRAVPGRVVALSQFNDTLNVYPHTSPPPLILKSWERNDPTQQIMVRDGQLTIKATIDVAIPKLR

SEQ ID NO: 100 - protein, 1047 aa, synthetic construct
LRVPPRPYVMPHSPAAGATIDVAIPKLRSQFNDTLNVYFAAPVALAAYPITGKLATIATFEMRYLQSLPAHLRPGLDELATYDKGILTVSRIDGTHQTLVAELIGDALFVQLHAPLRGVARGQTLVLYIRMWNQAALAMEVYQAETAVNTLFVAAWDKVMNLQAAEAFVAAYMPFLGLPPPWQPALRHPRSLFPEFSELFAAFPSFAGLRPTFDTRLMRAFQYDGVNDFPKYPLNVFATANAIAGILFLHSGLIALRVLDALTDKLAFHDQFVQALRAKLLSSILTYHAAIPAVVPELTPVAAAPPMLAVAPHTSPPPLILKSGFAIPIGQAMAMYAGPGSASLSRFMTDPHAMRDYVYPGHAIWWLARGLEFFQDGEQFGELLFTNPTGAFTLAIYHPQQFVYIEKPVAPSVMILAAYHPQQFIYAGSLSALLELENDNQLLYVHAASTRQALRVEMAAQQHVVIEAYTAAVELMPTQVELASQATDPLSLLIETVTQALQALTIPSFIPEDFTFLGRASGVLPRYYYQSGLSIVMMAVVGGALAYLVVKTLINATQLLKLLAKLAFPPEVHSAMLVSSPDVLTTYIGGTHPTTTYKAFDWDQAYRKPITYIMYNYPAMLGVRAATTMATYLLPLPNIPLLEYAARFITPVHPGYTATFLETPSQFFPFTGLNSLTDTRVATPSGLDGPGMSTSAYDIGLFYRYAWQNPVFSAQAAAIHEMFIDELKTNSSLLTSILTYPISPPGFPSLPYAMTLPPPVVAANRIQLLAAAVDIRETFRKELDEAVEAFDFPDSGTHSWELLDAHIPQLIADTDPLPVVFWERNDPTQQIAARALPLTSLTSAAERGPGQMLFYVWDFAEKFKEDVINDFVSSYAVADPMGAAFDYAAEVRAVPGRVVALMVRDGQLTIKGYTDDAGQTFVAAFQGAHARFGSVTPAVSQFNARTADGINYRVTYIPVVGHALSSLLDAHIPQLVAAADEVSTQVAQELDEISTNIILAPQINFFYIAVMPPAPGMVFSRPGLPVEYLQVPSPSMGSELRALVEVY

SEQ ID NO: 101 - protein, 1047 aa, synthetic construct
FYVWDFAEKFKEDVINDFVSSYAVADPMGAAFDYAAEVLRVPPRPYVMPHSPAAGAAIPAVVPELTPVAAAPPMLAVAIEKPVAPSVMDFPDSGTHSWERAVPGRVVALSLLDAHIPQLVRVLDALTDKLADTDPLPVVFWERNDPTQQIVRAATTMATYVSSPDVLTTYIAAADEVSTQVAIDELKTNSSLLTSILTYFAAPVALAAYPITGKLAAAVDIRETFRMAVVGGALAYLVVKTLINATQLLKLLAKLATIATFEMRYLQSLPAHLRPGLDELATIDVAIPKLRGGTHPTTTYKAFDWDQAYRKPITYIMYNYPAMLGYYQSGLSIVMAARALPLTSLTSAAERGPGQMLVAELIGDALFVQLHAPLRGVARGQTLVLYVEMAAQQHVVIEAYTAAVELMPTQVELADTRVATPSGLDGPGMSTSAYDIGLFYRYAWQNPVFAFHDQFVQALRAKLLSSILTYHSELRALVEVYPISPPGFPSLPYAMTLPPPVVAANRIQLLSRFMTDPHAMRGFAIPIGQAMAMYAGPGSASLATYDKGILTVGYTDDAGQTFVQELDEISTNIQAAEAFVAAYMPFLILAPQINFFYILAAYHPQQFIYAGSLSALLELENDNQLLYDYVYPGHAIWWLARGLEFFQDGEQFGELLFTNPTGAFTLAIYHPQQFVYIAVMPPAPGMVFSRPGLPVEYLQVPSPSMGAAFQGAHARFGRASGVLPRYGLPPPWQPALRHPRSLFPEFSELFAAFPSFAGLRPTFDTRLMRSRIDGTHQTLVAAWDKVMNLTYIPVVGHALSIRMWNQAALAMEVYQAETAVNTLFSQATDPLSLLIETVTQALQALTIPSFIPEDFTFLVHAASTRQALRSQFNDTLNVYLLPLPNIPLLEYAARFITPVHPGYTATFLETPSQFFPFTGLNSLTLLDAHIPQLISAQAAAIHEMFAFQYDGVNDFPKYPLNVFATANAIAGILFLHSGLIALPHTSPPPLILKSMVRDGQLTIKGSVTPAVSQFNARTADGINYRVAFPPEVHSAMLKELDEAVEAF

SEQ ID NO: 102 - protein, 1047 aa, synthetic construct
IMYNYPAMLGVRAATTMATYPISPPGFPSLPYAMTLPPPVVAANRIQLLFSRPGLPVEYLQVPSPSMGATYDKGILTVAARALPLTSLTSAAERGPGQMLFYVWDFAEKFKEDVINDFVSSYSRIDGTHQTLGYTDDAGQTFVAAFQGAHARFAFPPEVHSAMLVEMAAQQHVVIEAYTAAVELMPTQVELAMVRDGQLTIKSQATDPLSLLIETVTQALQALTIPSFIPEDFTFLDYVYPGHAIWWLARGLEFFQDGEQFGELLFTNPTGAFYYQSGLSIVMPHTSPPPLILKSSELRALVEVYVAELIGDALFVQLHAPLRGVARGQTLVLYAAAVDIRETFRMAVVGGALAYLVVKTLINATQLLKLLAKLGLPPPWQPALRHPRSLFPEFSELFAAFPSFAGLRPTFDTRLMRGFAIPIGQAMASLLDAHIPQLVSAQAAAIHEMFILAPQINFFYTLAIYHPQQFVYIAVMPPAPGMVRVLDALTDKLAFHDQFVQALRATIATFEMRYLQSLPAHLRPGLDELRAVPGRVVALVHAASTRQALRSQFNDTLNVYELENDNQLLYAKLLSSILTYHGSVTPAVSQFNARTADGINYRVSRFMTDPHAMRVAAWDKVMNLTYIPVVGHALSIRMWNQAALAMEVYQAETAVNTLFAFQYDGVNDFPKYPLNVFATANAIAGILFLHSGLIALADTDPLPVVFWERNDPTQQILLDAHIPQLIAVADPMGAAFDYAAEVLRVPPRPYVMPHSPAAGATIDVAIPKLRGGTHPTTTYKAFDWDQAYRKPITYIEKPVAPSVMILAAYHPQQFIYAGSLSALLDFPDSGTHSWEAAIPAVVPELTPVAAAPPMLAVAMYAGPGSASLQELDEISTNIGRASGVLPRYQAAEAFVAAYMPFLVSSPDVLTTYIAAADEVSTQVAKELDEAVEAFLLPLPNIPLLEYAARFITPVHPGYTATFLETPSQFFPFTGLNSLTDTRVATPSGLDGPGMSTSAYDIGLFYRYAWQNPVFIDELKTNSSLLTSILTYFAAPVALAAYPITGKL

SEQ ID NO: 103 - protein, 1047 aa, synthetic construct
FYVWDFAEKFKEDVINDFVSSYMYAGPGSASLVEMAAQQHVVIEAYTAAVELMPTQVELAGYTDDAGQTFVAAFQGAHARFSQATDPLSLLIETVTQALQALTIPSFIPEDFTFLAVADPMGAAFDYAAEVLRVPPRPYVMPHSPAAGAFPPEVHSAMLATIDVAIPKLRSQFNDTLNVYPISPPGFPSLPSRIDGTHQTLIRMWNQAALAMEVYQAETAVNTLFSAQAAAIHEMFIDELKTNSSLLTSILTYAAAVDIRETFRMAVVGGALAYLVVKTLINATQLLKLLAKLGLPPPWQPALRHPRSLFPEFSELFAAFPSFAGLRPTFDTRLMRAFQYDGVNDFPKYPLNVFATANAIAGILFLHSGLIALELENDNQLLYVAAWDKVMNLTYIPVVGHALSMVRDGQLTIKKELDEAVEAFVRAATTMATYVSSPDVLTTYIWERNDPTQQIAARALPLTSLTSAAERGPGQMLDYVYPGHAIWWLARGLEFFQDGEQFGELLFTNPTGAFPHTSPPPLILKSGRASGVLPRYGFAIPIGQAMAFSRPGLPVEYLQVPSPSMGQELDEISTNIQAAEAFVAAYMPFLAAADEVSTQVAVHAASTRQALRIEKPVAPSVMDTRVATPSGLDGPGMSTSAYDIGLFYRYAWQNPVFAFHDQFVQALRAAIPAVVPELTPVAAAPPMLAVAATYDKGILTVYYQSGLSIVMSLLDAHIPQLVGGTHPTTTYKAFDWDQAYRKPITYRVLDALTDKLATIATFEMRYLQSLPAHLRPGLDELYAMTLPPPVVAANRIQLLIMYNYPAMLGILAPQINFFYTLAIYHPQQFVYSRFMTDPHAMRLLDAHIPQLIADTDPLPVVFFAAPVALAAYPITGKLVAELIGDALFVQLHAPLRGVARGQTLVLYIAVMPPAPGMVRAVPGRVVALAKLLSSILTYHGSVTPAVSQFNARTADGINYRVILAAYHPQQFIYAGSLSALLLLPLPNIPLLEYAARFITPVHPGYTATFLETPSQFFPFTGLNSLTDFPDSGTHSWESELRALVEVY

SEQ ID NO: 104 - protein, 1047 aa, synthetic construct
FYVWDFAEKFKEDVINDFVSSYYYQSGLSIVMVEMAAQQHVVIEAYTAAVELMPTQVELAMVRDGQLTIKDTRVATPSGLDGPGMSTSAYDIGLFYRYAWQNPVFGSVTPAVSQFNARTADGINYRVQAAEAFVAAYMPFLILAPQINFFYILAAYHPQQFIYAGSLSALLLLPLPNIPLLEYAARFITPVHPGYTATFLETPSQFFPFTGLNSLTAARALPLTSLTSAAERGPGQMLWERNDPTQQIIEKPVAPSVMVSSPDVLTTYIGGTHPTTTYKAFDWDQAYRKPITYRVLDALTDKLATIATFEMRYLQSLPAHLRPGLDELLRVPPRPYVMPHSPAAGAFPPEVHSAMLATIDVAIPKLRFAAPVALAAYPITGKLADTDPLPVVFYAMTLPPPVVAANRIQLLSRFMTDPHAMRVHAASTRQALRATYDKGILTVTYIPVVGHALSAAAVDIRETFRMAVVGGALAYLVVKTLINATQLLKLLAKLGYTDDAGQTFVAAFQGAHARFSELRALVEVYVAELIGDALFVQLHAPLRGVARGQTLVLYIRMWNQAALAMEVYQAETAVNTLFAFQYDGVNDFPKYPLNVFATANAIAGILFLHSGLIALIMYNYPAMLGVRAATTMATYPISPPGFPSLPELENDNQLLYAKLLSSILTYHSQATDPLSLLIETVTQALQALTIPSFIPEDFTFLGFAIPIGQAMAAVADPMGAAFDYAAEVDFPDSGTHSWERAVPGRVVALMYAGPGSASLQELDEISTNIGRASGVLPRYGLPPPWQPALRHPRSLFPEFSELFAAFPSFAGLRPTFDTRLMRDYVYPGHAIWWLARGLEFFQDGEQFGELLFTNPTGAFTLAIYHPQQFVYIDELKTNSSLLTSILTYPHTSPPPLILKSAFHDQFVQALRAAIPAVVPELTPVAAAPPMLAVAIAVMPPAPGMVFSRPGLPVEYLQVPSPSMGSQFNDTLNVYLLDAHIPQLISAQAAAIHEMFVAAWDKVMNLSRIDGTHQTLSLLDAHIPQLVAAADEVSTQVAKELDEAVEAF

SEQ ID NO: 105 - protein, 1047 aa, synthetic construct
FYVWDFAEKFKEDVINDFVSSYKELDEAVEAFELENDNQLLYIMYNYPAMLGYYQSGLSIVMAARALPLTSLTSAAERGPGQMLIRMWNQAALAMEVYQAETAVNTLFAAAVDIRETFRSQFNDTLNVYAKLLSSILTYHGSVTPAVSQFNARTADGINYRVILAAYHPQQFIYAGSLSALLPHTSPPPLILKSSRFMTDPHAMRMVRDGQLTIKGFAIPIGQAMAVAAWDKVMNLTYIPVVGHALSIAVMPPAPGMVRAVPGRVVALFAAPVALAAYPITGKLATIATFEMRYLQSLPAHLRPGLDELGRASGVLPRYGLPPPWQPALRHPRSLFPEFSELFAAFPSFAGLRPTFDTRLMRSQATDPLSLLIETVTQALQALTIPSFIPEDFTFLAAFQGAHARFAFHDQFVQALRAAIPAVVPELTPVAAAPPMLAVAAVADPMGAAFDYAAEVLRVPPRPYVMPHSPAAGGYTDDAGQTFVQELDEISTNIVRAATTMATYPISPPGFPSLPDFPDSGTHSWEAFQYDGVNDFPKYPLNVFATANAIAGILFLHSGLIALRVLDALTDKLILAPQINFFYTLAIYHPQQFVYATYDKGILTVMYAGPGSASLSRIDGTHQTLDYVYPGHAIWWLARGLEFFQDGEQFGELLFTNPTGAFVHAASTRQALRVEMAAQQHVVIEAYTAAVELMPTQVELALLDAHIPQLIQAAEAFVAAYMPFLAAADEVSTQVADTRVATPSGLDGPGMSTSAYDIGLFYRYAWQNPVFIDELKTNSSLLTSILTYATIDVAIPKLRADTDPLPVVFYAMTLPPPVVAANRIQLLFSRPGLPVEYLQVPSPSMGSELRALVEVYLLPLPNIPLLEYAARFITPVHPGYTATFLETPSQFFPFTGLNSLTVAELIGDALFVQLHAPLRGVARGQTLVLYMAVVGGALAYLVVKTLINATQLLKLLAKLAFPPEVHSAMLWERNDPTQQIIEKPVAPSVMSLLDAHIPQLVSAQAAAIHEMFVSSPDVLTTYIGGTHPTTTYKAFDWDQAYRKPITY

SEQ ID NO: 106 - protein, 205 aa, Mycobacterium tuberculosis; identical to SEQ ID NO: 45

SEQ ID NO: 107 - protein, 70 aa, Mycobacterium tuberculosis
AVAASNNPELTTLTAALSGQLNPQVNLVDTLNSGQYTVFAPTNAAFSKLPASTIDELKTNSSLLTSILTY

SEQ ID NO: 108 - protein, 17 aa, Mycobacterium tuberculosis
GVSTANATVYMIDSVLM

SEQ ID NO: 109 - protein, 18 aa, Mycobacterium tuberculosis
PAAPVTTAAMADPAADLI

SEQ ID NO: 110 - protein, 71 aa, Mycobacterium tuberculosis
GKLNPDVNLVDTLNGGEYTVFAPTNAAFDKLPAATIDQLKTDAKLLSSILTYHVIAGQASPSRIDGTHQTL

SEQ ID NO: 111 - protein, 17 aa, Mycobacterium tuberculosis
GVHTANATVYMIDTVLM

SEQ ID NO: 112 - protein, 17 aa, Mycobacterium tuberculosis
QAETAVNTLFEKLEPMA

SEQ ID NO: 113 - protein, 10 aa, Mycobacterium tuberculosis
QLPPAATQTL

SEQ ID NO: 114 - protein, 10 aa, Mycobacterium tuberculosis
TRTPLMSQLI

SEQ ID NO: 115 - protein, 10 aa, Mycobacterium tuberculosis
MAADYDKLFR

SEQ ID NO: 116 - protein, 11 aa, Mycobacterium tuberculosis
QPFFDPSASFP

SEQ ID NO: 117 - protein, 10 aa, Mycobacterium tuberculosis
AASKPPTPPM

SEQ ID NO: 118 - protein, 12 aa, Mycobacterium tuberculosis
RRTAPAPPWAKM

SEQ ID NO: 119 - protein, 44 aa, Mycobacterium tuberculosis
WRHWVHALTRINLGLSPDEKYELDLHARVRRNPRGSYQIAVVGL

SEQ ID NO: 120 - protein, 10 aa, Mycobacterium tuberculosis
QVRADRILAL

SEQ ID NO: 121 - protein, 65 aa, Mycobacterium tuberculosis
ATIADVLAEKELSHYNDIRAHTSVNAVNLEVLPAPEYSSAQRALSDADWHFIADPASRFYNLVLA

SEQ ID NO: 122 - protein, 10 aa, Mycobacterium tuberculosis
FFDPLTRGVL

SEQ ID NO: 123 - protein, 63 aa, Mycobacterium tuberculosis
NHIMPGEPNVAVKDLVRHFEQQVQPGRVVVMPWDRHIAAGTEISLDLLDPIYKRKVLELAAAL

SEQ ID NO: 124 - protein, 30 aa, Mycobacterium tuberculosis
SQFNDTLNVYLTAHNALGSSLHTAGVDLAK

SEQ ID NO: 125 - protein, 43 aa, Mycobacterium tuberculosis
FIIDPTISAIDGLYDLLGIGIPNQGGILYSSLEYFEKALEELA

SEQ ID NO: 126 - protein, 21 aa, Mycobacterium tuberculosis
FVRPVAVDLTYIPVVGHALSA

SEQ ID NO: 127 - protein, 29 aa, Mycobacterium tuberculosis
MAVVGGALAYLVVKTLINATQLLKLLAKL

SEQ ID NO: 128 - protein, 11 aa, Mycobacterium tuberculosis
VHAASTRQALR

SEQ ID NO: 129 - protein, 10 aa, Mycobacterium tuberculosis
AEQVGGQSQL

SEQ ID NO: 130 - protein, 10 aa, Mycobacterium tuberculosis
ASKGTTTKKY

SEQ ID NO: 131 - protein, 10 aa, Mycobacterium tuberculosis
GWTADPIIGV

SEQ ID NO: 132 - protein, 89 aa, Mycobacterium tuberculosis
AETDTEQEIALFTVTNPPRTVSVSTLMDGRIDHVELSARVAWMSESQLASEILVIADLARQKAQSAQYAFILDRMSQQVDADEHRVALL

SEQ ID NO: 133 - protein, 11 aa, Mycobacterium tuberculosis
AEAEVFATRYS

SEQ ID NO: 134 - protein, 39 aa, Mycobacterium tuberculosis
AAKNAAQQLVLSADNMREYLAAGAKERQRLATSLRNAAK

SEQ ID NO: 135 - protein, 12 aa, Mycobacterium tuberculosis
AHFADGWNTFNL

SEQ ID NO: 136 - protein, 39 aa, Mycobacterium tuberculosis
DQQRQWILHMAKLSAAMAKQAQYVAQLHVWARREHPTYE

SEQ ID NO: 137 - protein, 37 aa, Mycobacterium tuberculosis
RLYAENPSARDQILPVYAEYQQRSEKVLTEYNNKAAL

SEQ ID NO: 138 - protein, 33 aa, Mycobacterium tuberculosis
RHPRSLFPEFSELFAAFPSFAGLRPTFDTRLMR

SEQ ID NO: 139 - protein, 35 aa, Mycobacterium tuberculosis
DTRVATPSGLDGPGMSTSAYDIGLFYRYAWQNPVF

SEQ ID NO: 140 - protein, 22 aa, Mycobacterium tuberculosis
AARALPLTSLTSAAERGPGQML

SEQ ID NO: 141 - protein, 18 aa, Mycobacterium tuberculosis
YAMTLPPPVVAANRIQLL

SEQ ID NO: 142 - protein, 11 aa, Mycobacterium tuberculosis
DFPDSGTHSWE

SEQ ID NO: 143 - protein, 12 aa, Mycobacterium tuberculosis
TLAIYHPQQFVY

SEQ ID NO: 144 - protein, 28 aa, Mycobacterium tuberculosis
VEMAAQQHVVIEAYTAAVELMPTQVELA

SEQ ID NO: 145 - protein, 17 aa, Mycobacterium tuberculosis
LRVPPRPYVMPHSPAAG

SEQ ID NO: 146 - protein, 24 aa, Mycobacterium tuberculosis
GGTHPTTTYKAFDWDQAYRKPITY

SEQ ID NO: 147 - protein, 10 aa, Mycobacterium tuberculosis
SRIDGTHQTL

SEQ ID NO: 148 - protein, 12 aa, Mycobacterium tuberculosis
PHTSPPPLILKS

SEQ ID NO: 149 - protein, 11 aa, Mycobacterium tuberculosis
VSSPDVLTTYI

SEQ ID NO: 150 - protein, 34 aa, Mycobacterium tuberculosis
SQATDPLSLLIETVTQALQALTIPSFIPEDFTFL

SEQ ID NO: 151 - protein, 14 aa, Mycobacterium tuberculosis
QAAEAFVAAYMPFL

SEQ ID NO: 152 - protein, 11 aa, Mycobacterium tuberculosis
PISPPGFPSLP

SEQ ID NO: 153 - protein, 11 aa, Mycobacterium tuberculosis
ATIDVAIPKLR

SEQ ID NO: 154 - protein, 11 aa, Mycobacterium tuberculosis
GYTDDAGQTFV

SEQ ID NO: 155 - protein, 11 aa, Mycobacterium tuberculosis
SAQAAAIHEMF

SEQ ID NO: 156 - protein, 10 aa, Mycobacterium tuberculosis
AAFQGAHARF

SEQ ID NO: 157 - protein, 10 aa, Mycobacterium tuberculosis
WERNDPTQQI

SEQ ID NO: 158 - protein, 10 aa, Mycobacterium tuberculosis
VAAWDKVMNL

SEQ ID NO: 159 - protein, 22 aa, Mycobacterium tuberculosis
FYVWDFAEKFKEDVINDFVSSY

SEQ ID NO: 160 - protein, 11 aa, Mycobacterium tuberculosis
AFPPEVHSAML

SEQ ID NO: 161 - protein, 37 aa, Mycobacterium tuberculosis
AFQYDGVNDFPKYPLNVFATANAIAGILFLHSGLIAL

SEQ ID NO: 162 - protein, 25 aa, Mycobacterium tuberculosis
ATIATFEMRYLQSLPAHLRPGLDEL

SEQ ID NO: 163 - protein, 24 aa, Mycobacterium tuberculosis
IRMWNQAALAMEVYQAETAVNTLF

SEQ ID NO: 164 - protein, 16 aa, Mycobacterium tuberculosis
FAAPVALAAYPITGKL

SEQ ID NO: 165 - protein, 10 aa, Mycobacterium tuberculosis
ATYDKGILTV

SEQ ID NO: 166 - protein, 11 aa, Mycobacterium tuberculosis
GFAIPIGQAMA

SEQ ID NO: 167 - protein, 20 aa, Mycobacterium tuberculosis
ILAAYHPQQFIYAGSLSALL

SEQ ID NO: 168 - protein, 11 aa, Mycobacterium tuberculosis
VHAASTRQALR

SEQ ID NO: 169 - protein, 10 aa, Mycobacterium tuberculosis
QELDEISTNI

SEQ ID NO: 170 - protein, 22 aa, Mycobacterium tuberculosis
GSVTPAVSQFNARTADGINYRV

SEQ ID NO: 171 - protein, 29 aa, Mycobacterium tuberculosis
VAELIGDALFVQLHAPLRGVARGQTLVLY

SEQ ID NO: 172 - protein, 23 aa, Mycobacterium tuberculosis
AAIPAVVPELTPVAAAPPMLAVA

SEQ ID NO: 173 - protein, 10 aa, Mycobacterium tuberculosis
ELENDNQLLY

SEQ ID NO: 174 - protein, 10 aa, Mycobacterium tuberculosis
LLDAHIPQLI

SEQ ID NO: 175 - protein, 37 aa, Mycobacterium tuberculosis
DYVYPGHAIWWLARGLEFFQDGEQFGELLFTNPTGAF

SEQ ID NO: 176 - protein, 29 aa, Mycobacterium tuberculosis
MAVVGGALAYLVVKTLINATQLLKLLAKL

SEQ ID NO: 177 - protein, 10 aa, Mycobacterium tuberculosis
KELDEAVEAF

SEQ ID NO: 178 - protein, 11 aa, Mycobacterium tuberculosis
SLLDAHIPQLV

SEQ ID NO: 179 - protein, 16 aa, Mycobacterium tuberculosis
AVADPMGAAFDYAAEV

SEQ ID NO: 180 - protein, 11 aa, Mycobacterium tuberculosis
TYIPVVGHALS

SEQ ID NO: 181 - protein, 10 aa, Mycobacterium tuberculosis
SQFNDTLNVY

SEQ ID NO: 182 - protein, 10 aa, Mycobacterium tuberculosis
RAVPGRVVAL

SEQ ID NO: 183 - protein, 11 aa, Mycobacterium tuberculosis
AKLLSSILTYH

SEQ ID NO: 184 - protein, 10 aa, Mycobacterium tuberculosis
IEKPVAPSVM

SEQ ID NO: 185 - protein, 11 aa, Mycobacterium tuberculosis
AAAVDIRETFR

SEQ ID NO: 186 - protein, 10 aa, Mycobacterium tuberculosis
GLPPPWQPAL

SEQ ID NO: 187 - protein, 19 aa, Mycobacterium tuberculosis
FSRPGLPVEYLQVPSPSMG

SEQ ID NO: 188 - protein, 10 aa, Mycobacterium tuberculosis
MVRDGQLTIK

SEQ ID NO: 189 - protein, 11 aa, Mycobacterium tuberculosis
AAADEVSTQVA

SEQ ID NO: 190 - protein, 45 aa, Mycobacterium tuberculosis
LLPLPNIPLLEYAARFITPVHPGYTATFLETPSQFFPFTGLNSLT

SEQ ID NO: 191 - protein, 10 aa, Mycobacterium tuberculosis
SELRALVEVY

SEQ ID NO: 192 - protein, 11 aa, Mycobacterium tuberculosis
IAVMPPAPGMV

SEQ ID NO: 193 - protein, 10 aa, Mycobacterium tuberculosis
VRAATTMATY

SEQ ID NO: 194 - protein, 10 aa, Mycobacterium tuberculosis
RVLDALTDKL

SEQ ID NO: 195 - protein, 10 aa, Mycobacterium tuberculosis
ILAPQINFFY

SEQ ID NO: 196 - protein, 10 aa, Mycobacterium tuberculosis
ADTDPLPVVF

SEQ ID NO: 197 - protein, 10 aa, Mycobacterium tuberculosis
GRASGVLPRY

SEQ ID NO: 198 - protein, 10 aa, Mycobacterium tuberculosis
IMYNYPAMLG

SEQ ID NO: 199 - protein, 17 aa, Mycobacterium tuberculosis
IDELKTNSSLLTSILTY

SEQ ID NO: 200 - protein, 10 aa, Mycobacterium tuberculosis
MYAGPGSASL

SEQ ID NO: 201 - protein, 10 aa, Mycobacterium tuberculosis
YYQSGLSIVM

SEQ ID NO: 202 - protein, 11 aa, Mycobacterium tuberculosis
SRFMTDPHAMR

SEQ ID NO: 203 - protein, 11 aa, Mycobacterium tuberculosis
AFHDQFVQALR

SEQ ID NO: 204 - protein, 76 aa, synthetic construct
MQIFVKTLTGKTITLEVEPSDTIENVKAKIQDKEGIPPDQQRLIFAGKQLEDGRTLSDYNIQKESTLHLVLRLRGA

SEQ ID NO: 205 - protein, 77 aa, synthetic construct
MQIFVKTLTGKTITLEVEPSDTIENVKAKIQDKEGIPPDQQRLIFAGKQLEDGRTLSDYNIQKESTLHLVLRLRGGR

SEQ ID NO: 206 - protein, 20 aa, Homo sapiens
MERLLDDRHDSGLDSMKDEE

SEQ ID NO: 207 - protein, 1137 aa, synthetic construct
MQIFVKTLTGKTITLEVEPSDTIENVKAKIQDKEGIPPDQQRLIFAGKQLEDGRTLSDYNIQKESTLHLVLRLRGAWRHWVHALTRINLGLSPDEKYELDLHARVRRNPRGSYQIAVVGLQLPPAATQTLPAAPVTTAAMADPAADLIAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAAETDTEQEIALFTVTNPPRTVSVSTLMDGRIDHVELSARVAWMSESQLASEILVIADLARQKAQSAQYAFILDRMSQQVDADEHRVALLGWTADPIIGVQVRADRILALAVAASNNPELTTLTAALSGQLNPQVNLVDTLNSGQYTVFAPTNAAFSKLPASTIDELKTNSSLLTSILTYNHIMPGEPNVAVKDLVRHFEQQVQPGRVVVMPWDRHIAAGTEISLDLLDPIYKRKVLELAAALQAETAVNTLFEKLEPMAAASKPPTPPMVHAASTRQALRAHFADGWNTFNLAEAEVFATRYSAAKNAAQQLVLSADNMREYLAAGAKERQRLATSLRNAAKRLYAENPSARDQILPVYAEYQQRSEKVLTEYNNKAALDQQRQWILHMAKLSAAMAKQAQYVAQLHVWARREHPTYEGVHTANATVYMIDTVLMASKGTTTKKYMAADYDKLFRATIADVLAEKELSHYNDIRAHTSVNAVNLEVLPAPEYSSAQRALSDADWHFIADPASRFYNLVLAFVRPVAVDLTYIPVVGHALSASQFNDTLNVYLTAHNALGSSLHTAGVDLAKFFDPLTRGVLTRTPLMSQLIFIIDPTISAIDGLYDLLGIGIPNQGGILYSSLEYFEKALEELAAEQVGGQSQLGKLNPDVNLVDTLNGGEYTVFAPTNAAFDKLPAATIDQLKTDAKLLSSILTYHVIAGQASPSRIDGTHQTLRRTAPAPPWAKMMAVVGGALAYLVVKTLINATQLLKLLAKLGVSTANATVYMIDSVLMQPFFDPSASFP

SEQ ID NO: 208 - protein, 1137 aa, synthetic construct
MQIFVKTLTGKTITLEVEPSDTIENVKAKIQDKEGIPPDQQRLIFAGKQLEDGRTLSDYNIQKESTLHLVLRLRGAAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAAAKNAAQQLVLSADNMREYLAAGAKERQRLATSLRNAAKASKGTTTKKYQLPPAATQTLFVRPVAVDLTYIPVVGHALSAAVAASNNPELTTLTAALSGQLNPQVNLVDTLNSGQYTVFAPTNAAFSKLPASTIDELKTNSSLLTSILTYNHIMPGEPNVAVKDLVRHFEQQVQPGRVVVMPWDRHIAAGTEISLDLLDPIYKRKVLELAAALFFDPLTRGVLTRTPLMSQLIGWTADPIIGVAASKPPTPPMRRTAPAPPWAKMWRHWVHALTRINLGLSPDEKYELDLHARVRRNPRGSYQIAVVGLQAETAVNTLFEKLEPMAQVRADRILALRLYAENPSARDQILPVYAEYQQRSEKVLTEYNNKAALDQQRQWILHMAKLSAAMAKQAQYVAQLHVWARREHPTYEGKLNPDVNLVDTLNGGEYTVFAPTNAAFDKLPAATIDQLKTDAKLLSSILTYHVIAGQASPSRIDGTHQTLAEQVGGQSQLPAAPVTTAAMADPAADLIMAADYDKLFRSQFNDTLNVYLTAHNALGSSLHTAGVDLAKFIIDPTISAIDGLYDLLGIGIPNQGGILYSSLEYFEKALEELAVHAASTRQALRGVHTANATVYMIDTVLMATIADVLAEKELSHYNDIRAHTSVNAVNLEVLPAPEYSSAQRALSDADWHFIADPASRFYNLVLAAHFADGWNTFNLAEAEVFATRYSAETDTEQEIALFTVTNPPRTVSVSTLMDGRIDHVELSARVAWMSESQLASEILVIADLARQKAQSAQYAFILDRMSQQVDADEHRVALLMAVVGGALAYLVVKTLINATQLLKLLAKLGVSTANATVYMIDSVLMQPFFDPSASFP

SEQ ID NO: 209 - protein, 1123 aa, synthetic construct
MQIFVKTLTGKTITLEVEPSDTIENVKAKIQDKEGIPPDQQRLIFAGKQLEDGRTLSDYNIQKESTLHLVLRLRGAFYVWDFAEKFKEDVINDFVSSYAVADPMGAAFDYAAEVLRVPPRPYVMPHSPAAGAAIPAVVPELTPVAAAPPMLAVAIEKPVAPSVMDFPDSGTHSWERAVPGRVVALSLLDAHIPQLVRVLDALTDKLADTDPLPVVFWERNDPTQQIVRAATTMATYVSSPDVLTTYIAAADEVSTQVAIDELKTNSSLLTSILTYFAAPVALAAYPITGKLAAAVDIRETFRMAVVGGALAYLVVKTLINATQLLKLLAKLATIATFEMRYLQSLPAHLRPGLDELATIDVAIPKLRGGTHPTTTYKAFDWDQAYRKPITYIMYNYPAMLGYYQSGLSIVMAARALPLTSLTSAAERGPGQMLVAELIGDALFVQLHAPLRGVARGQTLVLYVEMAAQQHVVIEAYTAAVELMPTQVELADTRVATPSGLDGPGMSTSAYDIGLFYRYAWQNPVFAFHDQFVQALRAKLLSSILTYHSELRALVEVYPISPPGFPSLPYAMTLPPPVVAANRIQLLSRFMTDPHAMRGFAIPIGQAMAMYAGPGSASLATYDKGILTVGYTDDAGQTFVQELDEISTNIQAAEAFVAAYMPFLILAPQINFFYILAAYHPQQFIYAGSLSALLELENDNQLLYDYVYPGHAIWWLARGLEFFQDGEQFGELLFTNPTGAFTLAIYHPQQFVYIAVMPPAPGMVFSRPGLPVEYLQVPSPSMGAAFQGAHARFGRASGVLPRYGLPPPWQPALRHPRSLFPEFSELFAAFPSFAGLRPTFDTRLMRSRIDGTHQTLVAAWDKVMNLTYIPVVGHALSIRMWNQAALAMEVYQAETAVNTLFSQATDPLSLLIETVTQALQALTIPSFIPEDFTFLVHAASTRQALRSQFNDTLNVYLLPLPNIPLLEYAARFITPVHPGYTATFLETPSQFFPFTGLNSLTLLDAHIPQLISAQAAAIHEMFAFQYDGVNDFPKYPLNVFATANAIAGILFLHSGLIALPHTSPPPLILKSMVRDGQLTIKGSVTPAVSQFNARTADGINYRVAFPPEVHSAMLKELDEAVEAF

SEQ ID NO: 210 - protein, 1123 aa, synthetic construct
MQIFVKTLTGKTITLEVEPSDTIENVKAKIQDKEGIPPDQQRLIFAGKQLEDGRTLSDYNIQKESTLHLVLRLRGAIMYNYPAMLGVRAATTMATYPISPPGFPSLPYAMTLPPPVVAANRIQLLFSRPGLPVEYLQVPSPSMGATYDKGILTVAARALPLTSLTSAAERGPGQMLFYVWDFAEKFKEDVINDFVSSYSRIDGTHQTLGYTDDAGQTFVAAFQGAHARFAFPPEVHSAMLVEMAAQQHVVIEAYTAAVELMPTQVELAMVRDGQLTIKSQATDPLSLLIETVTQALQALTIPSFIPEDFTFLDYVYPGHAIWWLARGLEFFQDGEQFGELLFTNPTGAFYYQSGLSIVMPHTSPPPLILKSSELRALVEVYVAELIGDALFVQLHAPLRGVARGQTLVLYAAAVDIRETFRMAVVGGALAYLVVKTLINATQLLKLLAKLGLPPPWQPALRHPRSLFPEFSELFAAFPSFAGLRPTFDTRLMRGFAIPIGQAMASLLDAHIPQLVSAQAAAIHEMFILAPQINFFYTLAIYHPQQFVYIAVMPPAPGMVRVLDALTDKLAFHDQFVQALRATIATFEMRYLQSLPAHLRPGLDELRAVPGRVVALVHAASTRQALRSQFNDTLNVYELENDNQLLYAKLLSSILTYHGSVTPAVSQFNARTADGINYRVSRFMTDPHAMRVAAWDKVMNLTYIPVVGHALSIRMWNQAALAMEVYQAETAVNTLFAFQYDGVNDFPKYPLNVFATANAIAGILFLHSGLIALADTDPLPVVFWERNDPTQQILLDAHIPQLIAVADPMGAAFDYAAEVLRVPPRPYVMPHSPAAGATIDVAIPKLRGGTHPTTTYKAFDWDQAYRKPITYIEKPVAPSVMILAAYHPQQFIYAGSLSALLDFPDSGTHSWEAAIPAVVPELTPVAAAPPMLAVAMYAGPGSASLQELDEISTNIGRASGVLPRYQAAEAFVAAYMPFLVSSPDVLTTYIAAADEVSTQVAKELDEAVEAFLLPLPNIPLLEYAARFITPVHPGYTATFLETPSQFFPFTGLNSLTDTRVATPSGLDGPGMSTSAYDIGLFYRYAWQNPVFIDELKTNSSLLTSILTYFAAPVALAAYPITGKL

SEQ ID NO: 211 - DNA, 4319 bp, synthetic construct
  source 1..4319: mol_type = other DNA
gggaataaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcagcggccgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 212 - protein, 1124 aa, synthetic construct
MRVTAPRTLILLLSGALALTETWAFYVWDFAEKFKEDVINDFVSSYAVADPMGAAFDYAAEVLRVPPRPYVMPHSPAAGAAIPAVVPELTPVAAAPPMLAVAIEKPVAPSVMDFPDSGTHSWERAVPGRVVALSLLDAHIPQLVRVLDALTDKLADTDPLPVVFWERNDPTQQIVRAATTMATYVSSPDVLTTYIAAADEVSTQVAIDELKTNSSLLTSILTYFAAPVALAAYPITGKLAAAVDIRETFRMAVVGGALAYLVVKTLINATQLLKLLAKLATIATFEMRYLQSLPAHLRPGLDELATIDVAIPKLRGGTHPTTTYKAFDWDQAYRKPITYIMYNYPAMLGYYQSGLSIVMAARALPLTSLTSAAERGPGQMLVAELIGDALFVQLHAPLRGVARGQTLVLYVEMAAQQHVVIEAYTAAVELMPTQVELADTRVATPSGLDGPGMSTSAYDIGLFYRYAWQNPVFAFHDQFVQALRAKLLSSILTYHSELRALVEVYPISPPGFPSLPYAMTLPPPVVAANRIQLLSRFMTDPHAMRGFAIPIGQAMAMYAGPGSASLATYDKGILTVGYTDDAGQTFVQELDEISTNIQAAEAFVAAYMPFLILAPQINFFYILAAYHPQQFIYAGSLSALLELENDNQLLYDYVYPGHAIWWLARGLEFFQDGEQFGELLFTNPTGAFTLAIYHPQQFVYIAVMPPAPGMVFSRPGLPVEYLQVPSPSMGAAFQGAHARFGRASGVLPRYGLPPPWQPALRHPRSLFPEFSELFAAFPSFAGLRPTFDTRLMRSRIDGTHQTLVAAWDKVMNLTYIPVVGHALSIRMWNQAALAMEVYQAETAVNTLFSQATDPLSLLIETVTQALQALTIPSFIPEDFTFLVHAASTRQALRSQFNDTLNVYLLPLPNIPLLEYAARFITPVHPGYTATFLETPSQFFPFTGLNSLTLLDAHIPQLISAQAAAIHEMFAFQYDGVNDFPKYPLNVFATANAIAGILFLHSGLIALPHTSPPPLILKSMVRDGQLTIKGSVTPAVSQFNARTADGINYRVAFPPEVHSAMLKELDEAVEAFGIVAGLAVLAVVVIGAVVAAVMCRRKSSGGKGGSYSQAACSDSAQGSDVSLTA

SEQ ID NO: 213 - RNA, 3565 nt, synthetic construct
  source 1..3565: mol_type = other RNA
acatttgcttctgacacaactgtgttcactagcaacctcaaacagccaccatgagagtgaccgccccgcggaccctcattctgcttctgtccggagcgctcgcactgactgagacttgggcattctacgtctgggacttcgccgagaagttcaaggaagatgtgatcaacgacttcgtgagctcgtacgctgtggccgaccctatgggagccgccttcgactacgccgccgaagtgctcagggtccctccgcggccttacgtgatgccacactccccggccgctggggccgctatccccgcggtggtccctgaactcaccccagtcgccgcagccccccctatgctggccgtggccattgagaagcctgtcgccccgagcgtgatggatttcccggattccggaacccatagctgggaacgcgctgtgcccggacgggtggtggcattgagcctgctcgacgcccacattccccaactcgtgcgcgtcctggacgccctgactgataagctggccgacaccgatcccctgcccgttgtgttctgggagaggaacgatccaactcagcaaattgtccgggcggctaccactatggcaacatacgtgtcgtcgccggacgtgcttacaacctacatagcggcggcggacgaagtgtctacccaagtggccatcgacgaacttaagaccaactccagtttgctgacctccatcctgacctacttcgcggcccccgtggccctggctgcctaccctatcaccggaaagctcgccgccgcggtggatatcagagaaaccttccggatggcagtcgtgggtggagccttggcctacctggtggtcaagaccctgatcaacgccacgcagttgctgaagctgctcgcgaagctggcgaccatcgccactttcgagatgcggtatctgcagtctctgcccgctcacctccggcccgggctcgacgagctggccaccatcgacgtggcgatcccgaaactgagaggaggcacccacccgaccaccacttacaaggcattcgattgggatcaggcctaccggaagcctattacctacattatgtataactacccggccatgctgggctattaccagtcgggcctgagcatcgtgatggccgcacgggctctgcccctgacctccttgacctcggccgccgagcgcggacccggacagatgttggtggcagaactcatcggagatgcactgttcgtgcaactgcacgcgccgctccgcggcgtggctcgaggacagaccctggtgctctacgtggaaatggccgcccagcagcatgtggtcatcgaggcctacaccgccgccgtggagctgatgcccactcaagtggagctggcagacactcgcgtggccaccccgagcggcctcgacggcccgggaatgtcgacctccgcgtacgacatcggtctgttctaccgctacgcctggcaaaaccctgtgttcgcgttccatgaccagttcgtacaggctctgcgcgccaaactgctgtccagcattctgacctaccattccgaactgcgggcgcttgtggaggtctaccccatctcccccccgggtttcccatcattaccttacgccatgactctcccaccgcccgtggtcgccgcgaatcgcattcagctgctaagccgcttcatgaccgacccacacgcgatgaggggattcgccatccccattgggcaggccatggctatgtacgctggcccgggatccgcctccctggccacctacgacaagggaatcctgactgtgggctacacggacgatgcaggacagactttcgtgcaagaactggatgaaatcagcactaacatccaggcagccgaggcctttgtggcggcctacatgcctttcctgatcctcgcgccgcaaatcaatttcttctacattctggccgcttaccacccgcagcagttcatctacgccggatcgctgtcagcactgctggagctggagaacgacaaccagctcctctacgactacgtctaccctggccatgcgatttggtggcttgctcgcggcttggagtttttccaagacggcgaacagttcggtgaactcctgttcaccaaccctacgggagccttcactctggccatctatcacccccaacagttcgtgtatatcgccgtgatgcccccggcccccggaatggtcttttcgcggccgggccttccggtggagtatctccaggtcccttccccttcgatgggggccgccttccaaggcgcccacgcacggtttggtcgcgcgtcaggagtgctgccgcgatacggactgcccccgccatggcagccggccctccgccacccaagatcgctgttccccgaattctcggagctctttgctgccttcccctccttcgccgggcttaggccaaccttcgacacccggctcatgagatcgcgcatcgacggtactcaccagaccctcgtggccgcctgggacaaggtcatgaacctgacttacatccctgtcgtgggacacgccctttccattcggatgtggaaccaggcagcgctggccatggaagtgtaccaggccgaaaccgccgtgaacaccctgttttcccaagccactgacccgctttccctccttatcgaaacagtgacccaggcactgcaagctctgaccattccaagcttcatccctgaggatttcactttcctggtgcacgccgccagcaccagacaggccctgagatcccagttcaacgacaccctgaacgtgtatctgctgcccctgcccaacattccgctcctggaatacgccgctagattcatcactcctgtgcatcctgggtacaccgccaccttcctggaaaccccgagccagttcttcccgttcacgggtttgaactccctgaccctgctggacgctcatattcctcaactcattagcgcacaggccgccgccattcacgaaatgtttgccttccaatacgacggagtgaatgactttccgaagtaccccctcaacgtgttcgccaccgcgaacgccattgcggggatcctcttcctgcactccgggctgatcgccctgcctcacacttccccgcccccactgatccttaagtcaatggtccgcgacggacagctgactatcaaggggtccgtcactcccgcggtgtcccagtttaatgcgcgcactgcggacggcatcaactatcgggtggcgtttccgccggaagtgcactcagccatgctgaaggagttggacgaggcggtggaggccttcggaattgtggccggccttgccgtgctcgccgtggtcgtgatcggcgccgtggtggccgccgtcatgtgccggagaaagagctcaggaggaaagggtggcagctacagccaggcagcatgttcagattcggcccagggctccgatgtgtccctgaccgcctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 214 - RNA, 3562 nt, synthetic construct
  source 1..3562: mol_type = other RNA
acatttgcttctgacacaactgtgttcactagcaacctcaaacagccaccatgcaaatcttcgtcaagaccctcaccggaaagaccattacactggaagtcgagccctccgatactatcgagaacgtgaaggccaagatccaggacaaggagggcattcccccggaccagcagcgcctgatcttcgcggggaagcagctggaagatggacggaccctgtccgactacaacatccagaaggaatccactcttcacctcgtgctgcgcctgagaggagcgttctacgtgtgggactttgccgagaagttcaaggaggacgtgatcaacgacttcgtgtcatcctacgccgtggccgaccctatgggagccgccttcgactacgccgccgaagtcctgcgggttccgccccgcccctatgtcatgccgcactcccccgccgccggagcggcgattcctgcggtggtgcccgaactgacgccggtggcagccgccccacccatgctggccgtggcaatcgagaagcccgtagccccaagcgtcatggacttcccggattcgggaacacacagctgggaaagagccgtccccggacgggtggtggccctgagtttgcttgatgcccacattccccaactcgtgcgcgtgctggatgccctgaccgacaaactcgccgatacggaccccctgcctgtcgtgttttgggagagaaacgacccgacacagcagattgtgcgggccgccactaccatggctacttacgtgtcctcccccgatgtgctcaccacctatattgccgcggccgacgaagtgtcgacccaggtcgccatcgatgaactgaaaaccaatagctcactgctgacctccatcctcacttacttcgccgcccccgtggcactggcagcctaccctatcactggaaagctggctgcggctgtggatatcagagagactttcaggatggcagtggtcggaggcgccctggcgtacctggtggtcaaaacgctgatcaacgccacccagctgctcaagctcttggcgaaactggccaccattgccactttcgagatgcggtacctccagtcacttcccgcccacttgaggccgggcctggatgaactggctaccatcgacgtggccatccccaagctgaggggcggtactcatcccactactacctacaaggcgtttgattgggatcaggcataccgcaagcccattacctacatcatgtataactaccctgcgatgctcggttactaccagtccggcctgtcgattgtgatggccgcgcgggcactgccattaacctcactgacctccgccgctgagcggggaccggggcagatgctcgtggccgagctgattggcgacgccctgttcgtgcaactgcacgcgccgcttcggggcgtggcccgcggacagactctcgtcctttacgtggaaatggcggctcagcagcacgtggtcatcgaggcctacaccgctgcggtggaactcatgcctactcaagtggagctggcggacactagagtcgccaccccatcgggtctggacggtcctgggatgagcacgtccgcatacgacatcggcctgttctaccgctatgcctggcaaaaccctgtgttcgcgttccacgaccagttcgtgcaggctttgagagccaagctgctgtcctcgatcctgacctaccactcggaactgcgagccttggtggaggtgtaccctatttcccctcctggtttcccgtccctgccttacgccatgaccctgccgccaccagtggtggccgccaacaggatacagttgctgtcccgcttcatgactgatccccacgccatgagggggttcgcaattccgattggccaggccatggctatgtacgccggccctggaagcgcatccctcgccacctacgacaagggaatcctgaccgtgggatacaccgacgacgccggccagaccttcgtgcaagagctcgacgaaatctccaccaacatccaagccgccgaggccttcgtcgccgcctacatgcctttcctgatcctggctccgcaaatcaacttcttctacatccttgccgcataccacccacaacagttcatctacgccgggtcgttgagcgccttgctggaacttgagaacgacaatcagctgctctacgactacgtgtaccctggacacgccatttggtggctcgcacgcgggctcgagtttttccaagacggggaacagtttggcgaactgctttttactaacccgaccggcgccttcaccctggccatctaccatccgcaacagttcgtgtacatcgccgtgatgccgccggcgccagggatggtgttctcccggcctgggctgccggtggaatacctccaagtgccgtcaccaagcatgggagcggcattccagggcgctcacgctcggttcggaagggccagcggagtgctgccgcgctacggtctgcctccgccatggcagcctgccctgcggcatccgcgctccctcttccctgaattctccgaactattcgccgctttcccctcgttcgccggcctgcggccgaccttcgacacccggctgatgcggtccagaatcgatggaacccatcagaccctggtggcagcgtgggataaggtcatgaacctcacctacatccccgtcgtgggtcacgcgttgtccattcgcatgtggaaccaggctgcgctggctatggaagtgtaccaggcagaaaccgcagtgaacactctgttttcacaagcgaccgaccccctgtccctgctgatcgaaaccgtgactcaggccctgcaagcccttaccatcccgtccttcattcctgaggacttcacctttttggtgcacgccgcctccacccgccaagcgctgagatcccagttcaacgacaccctgaatgtctatctcctccctctgccgaacattcccctgcttgaatacgctgcccggttcatcacccccgtgcaccccgggtatactgccaccttcctggagactccgagccagttcttcccgtttaccggcctgaattccctgaccctcctcgacgcccacatcccacaactgatcagcgcgcaggccgccgccattcatgagatgtttgcgttccaatacgacggcgtgaacgacttcccgaagtaccccctgaacgtgttcgccaccgcgaacgctatcgccggaatcctcttcctccattcgggactcattgcgctgcctcatacttctccgcccccattgatcctgaagtcgatggtccgcgacggacagctgactatcaagggttctgtgactccggcagtcagccagtttaacgctagaaccgctgacggcattaactaccgggtggccttccctccggaagtgcacagcgccatgctgaaggagctggatgaggccgtggaggccttctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 215 - protein, 1138 aa, synthetic construct
MRVTAPRTLILLLSGALALTETWAWRHWVHALTRINLGLSPDEKYELDLHARVRRNPRGSYQIAVVGLQLPPAATQTLPAAPVTTAAMADPAADLIAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAAETDTEQEIALFTVTNPPRTVSVSTLMDGRIDHVELSARVAWMSESQLASEILVIADLARQKAQSAQYAFILDRMSQQVDADEHRVALLGWTADPIIGVQVRADRILALAVAASNNPELTTLTAALSGQLNPQVNLVDTLNSGQYTVFAPTNAAFSKLPASTIDELKTNSSLLTSILTYNHIMPGEPNVAVKDLVRHFEQQVQPGRVVVMPWDRHIAAGTEISLDLLDPIYKRKVLELAAALQAETAVNTLFEKLEPMAAASKPPTPPMVHAASTRQALRAHFADGWNTFNLAEAEVFATRYSAAKNAAQQLVLSADNMREYLAAGAKERQRLATSLRNAAKRLYAENPSARDQILPVYAEYQQRSEKVLTEYNNKAALDQQRQWILHMAKLSAAMAKQAQYVAQLHVWARREHPTYEGVHTANATVYMIDTVLMASKGTTTKKYMAADYDKLFRATIADVLAEKELSHYNDIRAHTSVNAVNLEVLPAPEYSSAQRALSDADWHFIADPASRFYNLVLAFVRPVAVDLTYIPVVGHALSASQFNDTLNVYLTAHNALGSSLHTAGVDLAKFFDPLTRGVLTRTPLMSQLIFIIDPTISAIDGLYDLLGIGIPNQGGILYSSLEYFEKALEELAAEQVGGQSQLGKLNPDVNLVDTLNGGEYTVFAPTNAAFDKLPAATIDQLKTDAKLLSSILTYHVIAGQASPSRIDGTHQTLRRTAPAPPWAKMMAVVGGALAYLVVKTLINATQLLKLLAKLGVSTANATVYMIDSVLMQPFFDPSASFPGIVAGLAVLAVVVIGAVVAAVMCRRKSSGGKGGSYSQAACSDSAQGSDVSLTA

SEQ ID NO: 216 - RNA, 3607 nt, synthetic construct
  source 1..3607: mol_type = other RNA
acatttgcttctgacacaactgtgttcactagcaacctcaaacagccaccatgcgcgtgactgcaccccgcactttaatcttgctgctgtctggagccctcgcgctgaccgagacatgggcatggcgccactgggtccacgccctcacccggatcaacctgggactgtcacccgacgagaaatacgaactggacctccacgcccgggtccgccggaacccaagaggatcctaccagattgccgtcgtgggactgcagctgccccctgcggccactcagaccctccccgccgccccggtcaccactgcggcaatggccgacccagctgcggacttgatcgcgcctaagacttattgcgaggaactgaagggcaccgacactggtcaagcgtgccagattcagatgtccgaccctgcctacaacattaacatctccctgccgtcgtactatccggaccaaaagtcgctcgagaactacatcgcccagacccgcgataagtttctttcggccgcgacctccagcactccgagggaggcgccatacgaactcaatatcactagcgcgacctaccagtccgccatccctccgcggggtacccaggccgtggtgctcaaggtgtaccagaacgccggaggcactcaccctaccaccacctacaaggccttcgactgggaccaggcgtaccgcaagcccattacctatgatacgctgtggcaggccgacaccgatcctctgccggtggtgttcccaatcgtgcagggcgaactcagcaagcagaccgggcaacaagtctccatcgctcccaacgctggcctggaccccgtgaactaccagaacttcgccgtgaccaacgacggggtcatcttcttcttcaaccctggagaactgctgcctgaagccgctggacctactcaagtgctggtgccccgctcggcaatcgactccatgctcgccgccgagactgatacagaacaggagatcgccctgttcactgtgaccaacccacccagaaccgtgtcagtgtcaactctcatggacggaaggattgatcatgtggagctgtccgcgcgggtggcctggatgagcgaatcccagctggcctccgaaatcctcgtgatcgccgacctggctcgacagaaggcccagtccgcacagtacgccttcattctcgaccggatgagccaacaggtcgacgcggatgagcatagggtggccctcctgggctggactgcggatccgatcatcggcgtccaagtccgcgccgatcgcatcctggctcttgccgtggccgcctccaacaaccctgaactgaccaccttgacggccgccctgtcgggccaactcaatcctcaagtgaacttggtggacacgctgaactccgggcagtacaccgtgtttgcccctactaacgccgctttctcaaaactcccggcctccactattgacgagctcaaaaccaactccagcctgttgacttccattttgacctacaatcacattatgccgggcgaaccgaacgtggccgtgaaggatctcgtcagacacttcgaacaacaggtccaaccgggacgggtggtcgtgatgccttgggaccggcatattgcggctggaaccgaaatctccctggatctgcttgaccctatatacaagcggaaggtcctggaactggctgccgcgctgcaagccgaaaccgccgtgaacaccctgtttgaaaagttggagcccatggcagcggcgtcaaagcccccgaccccgcctatggtgcacgcagccagcaccagacaggcactgagggcacacttcgccgacggttggaacactttcaacttggccgaggccgaagtgttcgccactcggtacagcgctgccaagaacgccgcgcagcagctggtgctgtccgccgacaacatgcgcgagtacctggccgcgggtgcaaaggaaaggcagcggctcgccaccagtctgcgcaacgccgccaagcggctgtacgcggagaatccgtccgcccgcgatcagatcctcccggtgtacgctgaataccagcagcggtccgagaaagtgcttaccgagtacaacaacaaggccgccctcgaccagcaacggcaatggatcctgcatatggccaaactgagcgccgccatggcaaagcaggctcagtacgtggcccagctgcacgtctgggcgcggagagagcacccaacctacgaaggggtgcacactgcaaacgccactgtgtatatgattgacaccgtgctcatggcttcaaagggcaccacgaccaagaagtacatggcagccgattacgacaagctgttccgggccactatcgcggacgtgctcgccgagaaggagctaagccattacaatgacatccgggcccatacctccgtgaacgcagtgaacctggaagtgcttcctgcccccgaatactcgtcggctcagcgggccctgtcagacgccgattggcactttatagccgatccagcctcccgcttctacaacctggtcctggcgttcgtgcggcccgtggccgtcgatctcacctacattccggtggtcggtcatgcgctcagcgcatcacagttcaacgacaccctgaatgtgtatctgaccgcccacaacgccctggggtccagcctgcacaccgccggagtggacctcgccaagtttttcgatccgctgacccggggcgtgctgacaagaaccccactgatgtcccagctgattttcattatcgaccccactatctccgcgatcgacggactctacgaccttttgggaatcggcatcccgaaccagggtggcatcctctactcttcactcgagtatttcgaaaaagccctggaggagctggccgcagaacaagtcggcgggcagtcacagctcggcaaactgaaccctgacgtcaacctcgtggataccctcaacgggggagaatacaccgttttcgcgcccactaacgcagctttcgataagctgccggctgccaccattgaccagctcaagactgacgctaagcttctgtcgtcgatcctgacctaccacgtcatcgcgggccaggcctccccctcgcgcatcgacggaacccatcagaccctaagaagaactgccccggccccgccctgggccaagatgatggccgtggtcgggggagctctggcctacctggtggtcaagaccctgatcaatgccacccaactgctgaagctgctcgcaaagctgggggtgtccaccgccaacgcgaccgtgtacatgatcgatagcgtgctgatgcagcctttctttgacccttccgcctccttccccggaatcgtggctggccttgccgtgttggccgtcgtcgtgattggcgctgtggtggctgctgtgatgtgtcgccgcaagagctcggggggaaagggaggaagctattcccaagcggcctgctcggacagcgctcagggctcggacgtgtcgctgaccgcgtgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 217 - RNA, 3604 nt, synthetic construct
  source 1..3604: mol_type = other RNA
acatttgcttctgacacaactgtgttcactagcaacctcaaacagccaccatgcaaatcttcgtcaagaccctcaccggcaaaaccattaccctggaagtcgaacctagcgacaccatcgagaacgtgaaggccaagatccaggataaggaggggattccgccggaccagcagaggcttatctttgccgggaagcagctcgaggacggaaggaccctgtccgactacaacatccagaaggagagcacactgcatttggtgctccgcctgcggggcgcctggagacattgggtgcatgccctgacacgcattaacctgggcctgtcccccgatgagaaatacgaactggacctccatgcgcgcgtgcgccggaaccctcggggatcgtaccagatcgccgtcgtggggctgcagcttccaccggcggccactcagaccctccctgccgcacctgtgactaccgccgcaatggctgaccccgccgccgacttgattgcgcccaaaacttactgcgaagaactcaaggggaccgacacgggacaggcttgccagattcagatgagtgaccctgcctacaacattaacatctcccttccttcttactatccggatcagaagtcactcgagaactacatcgcccaaacccgggataagtttctgtccgccgccacttcatccacccctcgcgaagccccctacgagctgaatatcacctcggcgacctaccagtccgctatcccgcctcgggggactcaggctgtggtgctcaaagtgtaccagaacgccggcggtacccacccgaccactacgtacaaggccttcgattgggaccaggcctaccggaagccaatcacctatgacacgctgtggcaggccgacaccgacccgcttcctgtggtgttcccgatcgtgcagggcgaattgtccaagcaaactggccaacaagtgtccatagcgccgaacgccgggttggaccccgtgaactaccagaacttcgccgtgaccaacgacggagtgattttcttcttcaaccctggagaactgctgccggaggccgccggcccgacccaagtgctcgtgccgagatcggccattgattccatgctcgccgcggaaaccgatacggagcaggagattgccctgttcactgtgacaaacccaccgaggactgtgtccgtgtccactctgatggacggacggatcgaccatgtggagctaagcgccagggtggcctggatgagcgaatcacagctcgcatccgagattctggtcatcgctgacctggccagacagaaggcccagtccgcacagtacgcattcatcctggaccgcatgtcgcaacaagtggatgccgacgagcacagggtggcgttgctgggatggaccgcagaccctatcatcggcgttcaagtccgcgcagaccgcatcctcgcgctggcggtggccgcctcgaacaacccggaactgaccaccctgaccgccgctctgtccggccaactgaacccacaagtcaacctcgtggacaccttgaattccggacagtacaccgtgttcgccccgaccaacgctgcgttctccaagctgccggcctcaactatcgacgaactcaagacaaactccagcctgctgacctcgatcctcacctacaaccacattatgccgggagaacctaacgtggccgtcaaagaccttgtgcgccacttcgagcaacaggtccagcccggccgagtggtggtcatgccctgggatcggcacatcgccgccggcactgaaatttcgctggatctcctggacccaatctacaagcggaaggtcctggagctggcagcggcgctccaagccgaaaccgccgtgaacactctgttcgagaagctggagcctatggctgccgcctccaagcctcccactcctcccatggtgcacgcagctagcaccagacaggctctgagagcccacttcgccgacggttggaacaccttcaacctggccgaagctgaagtgttcgcgactagatactccgcggcaaagaatgcagctcagcagctggtgctgtcagccgataacatgcgggagtacctggccgcgggagccaaggaaagacagaggctggcaacctcgctgagaaacgccgcgaagcgcctgtatgctgaaaacccctcagcccgcgaccagatcctgccggtgtacgccgaataccagcagcggagcgaaaaggtcctcaccgagtacaacaacaaggcggcgctggatcagcagcggcagtggattttgcacatggctaagttatccgctgccatggccaagcaggcccaatacgtggcccagcttcacgtctgggctcggcgcgaacaccctacctacgaaggggtgcacaccgccaacgctacggtgtacatgatcgataccgtgctgatggcctcgaagggtactaccaccaagaagtacatggcagccgactacgacaagctgttccgcgccactatcgcggacgtgctggccgagaaggagctgagccattacaacgacatccgggcccacacctctgtgaacgccgtgaatctcgaggtgctgcccgctccggagtattcctcggcgcaaagagccttgagcgatgccgattggcacttcatcgcggaccccgcctcccggttctacaatctggtactggcctttgtgcggcccgtcgctgtcgatctgacttacatccccgtggtcggacacgccctgagcgccagccagttcaacgacactctgaacgtgtacctgactgcccacaacgcccttggaagcagcctgcacactgccggagtggacctcgccaagttctttgatccccttacccgcggggtgttgaccaggactccgctcatgtcccaactgatcttcatcatcgaccccaccattagcgccattgacggcctctacgaccttctcggtattggtatcccaaatcagggtggcatcctgtactcctcactggaatacttcgaaaaggccctggaggagctcgccgccgaacaagtcggtggccagtcgcagctgggaaagctgaacccagatgtgaacctggtggacaccctgaacggaggagagtacactgtgtttgccccaaccaacgccgcgttcgacaagctccccgcggcaaccatcgaccagctgaaaactgatgccaagctgctctcctccattctgacctaccatgtcatcgcgggacaagcatccccctcccgaatcgacgggactcaccagacccttcggcggactgcgccagcacccccgtgggcgaagatgatggcagtggtcggaggcgccctggcttacctggtggtcaagaccttgatcaatgccacccagctgctcaagctgctggccaagctgggagtgtccaccgcgaacgccaccgtgtatatgattgattcggtgctgatgcagcctttcttcgacccgagcgcgtcattcccctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 218 - protein, 1081 aa, synthetic construct
MERLLDDRHDSGLDSMKDEEWRHWVHALTRINLGLSPDEKYELDLHARVRRNPRGSYQIAVVGLQLPPAATQTLPAAPVTTAAMADPAADLIAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAAETDTEQEIALFTVTNPPRTVSVSTLMDGRIDHVELSARVAWMSESQLASEILVIADLARQKAQSAQYAFILDRMSQQVDADEHRVALLGWTADPIIGVQVRADRILALAVAASNNPELTTLTAALSGQLNPQVNLVDTLNSGQYTVFAPTNAAFSKLPASTIDELKTNSSLLTSILTYNHIMPGEPNVAVKDLVRHFEQQVQPGRVVVMPWDRHIAAGTEISLDLLDPIYKRKVLELAAALQAETAVNTLFEKLEPMAAASKPPTPPMVHAASTRQALRAHFADGWNTFNLAEAEVFATRYSAAKNAAQQLVLSADNMREYLAAGAKERQRLATSLRNAAKRLYAENPSARDQILPVYAEYQQRSEKVLTEYNNKAALDQQRQWILHMAKLSAAMAKQAQYVAQLHVWARREHPTYEGVHTANATVYMIDTVLMASKGTTTKKYMAADYDKLFRATIADVLAEKELSHYNDIRAHTSVNAVNLEVLPAPEYSSAQRALSDADWHFIADPASRFYNLVLAFVRPVAVDLTYIPVVGHALSASQFNDTLNVYLTAHNALGSSLHTAGVDLAKFFDPLTRGVLTRTPLMSQLIFIIDPTISAIDGLYDLLGIGIPNQGGILYSSLEYFEKALEELAAEQVGGQSQLGKLNPDVNLVDTLNGGEYTVFAPTNAAFDKLPAATIDQLKTDAKLLSSILTYHVIAGQASPSRIDGTHQTLRRTAPAPPWAKMMAVVGGALAYLVVKTLINATQLLKLLAKLGVSTANATVYMIDSVLMQPFFDPSASFP

SEQ ID NO: 219 - RNA, 3436 nt, synthetic construct
  source 1..3436: mol_type = other RNA
acatttgcttctgacacaactgtgttcactagcaacctcaaacagccaccatggaaaggcttctggacgataggcatgactccggcttggatagcatgaaggacgaggagtggagacattgggtgcatgccctgacacgcattaacctgggcctgtcccccgatgagaaatacgaactggacctccatgcgcgcgtgcgccggaaccctcggggatcgtaccagatcgccgtcgtggggctgcagcttccaccggcggccactcagaccctccctgccgcacctgtgactaccgccgcaatggctgaccccgccgccgacttgattgcgcccaaaacttactgcgaagaactcaaggggaccgacacgggacaggcttgccagattcagatgagtgaccctgcctacaacattaacatctcccttccttcttactatccggatcagaagtcactcgagaactacatcgcccaaacccgggataagtttctgtccgccgccacttcatccacccctcgcgaagccccctacgagctgaatatcacctcggcgacctaccagtccgctatcccgcctcgggggactcaggctgtggtgctcaaagtgtaccagaacgccggcggtacccacccgaccactacgtacaaggccttcgattgggaccaggcctaccggaagccaatcacctatgacacgctgtggcaggccgacaccgacccgcttcctgtggtgttcccgatcgtgcagggcgaattgtccaagcaaactggccaacaagtgtccatagcgccgaacgccgggttggaccccgtgaactaccagaacttcgccgtgaccaacgacggagtgattttcttcttcaaccctggagaactgctgccggaggccgccggcccgacccaagtgctcgtgccgagatcggccattgattccatgctcgccgcggaaaccgatacggagcaggagattgccctgttcactgtgacaaacccaccgaggactgtgtccgtgtccactctgatggacggacggatcgaccatgtggagctaagcgccagggtggcctggatgagcgaatcacagctcgcatccgagattctggtcatcgctgacctggccagacagaaggcccagtccgcacagtacgcattcatcctggaccgcatgtcgcaacaagtggatgccgacgagcacagggtggcgttgctgggatggaccgcagaccctatcatcggcgttcaagtccgcgcagaccgcatcctcgcgctggcggtggccgcctcgaacaacccggaactgaccaccctgaccgccgctctgtccggccaactgaacccacaagtcaacctcgtggacaccttgaattccggacagtacaccgtgttcgccccgaccaacgctgcgttctccaagctgccggcctcaactatcgacgaactcaagacaaactccagcctgctgacctcgatcctcacctacaaccacattatgccgggagaacctaacgtggccgtcaaagaccttgtgcgccacttcgagcaacaggtccagcccggccgagtggtggtcatgccctgggatcggcacatcgccgccggcactgaaatttcgctggatctcctggacccaatctacaagcggaaggtcctggagctggcagcggcgctccaagccgaaaccgccgtgaacactctgttcgagaagctggagcctatggctgccgcctccaagcctcccactcctcccatggtgcacgcagctagcaccagacaggctctgagagcccacttcgccgacggttggaacaccttcaacctggccgaagctgaagtgttcgcgactagatactccgcggcaaagaatgcagctcagcagctggtgctgtcagccgataacatgcgggagtacctggccgcgggagccaaggaaagacagaggctggcaacctcgctgagaaacgccgcgaagcgcctgtatgctgaaaacccctcagcccgcgaccagatcctgccggtgtacgccgaataccagcagcggagcgaaaaggtcctcaccgagtacaacaacaaggcggcgctggatcagcagcggcagtggattttgcacatggctaagttatccgctgccatggccaagcaggcccaatacgtggcccagcttcacgtctgggctcggcgcgaacaccctacctacgaaggggtgcacaccgccaacgctacggtgtacatgatcgataccgtgctgatggcctcgaagggtactaccaccaagaagtacatggcagccgactacgacaagctgttccgcgccactatcgcggacgtgctggccgagaaggagctgagccattacaacgacatccgggcccacacctctgtgaacgccgtgaatctcgaggtgctgcccgctccggagtattcctcggcgcaaagagccttgagcgatgccgattggcacttcatcgcggaccccgcctcccggttctacaatctggtactggcctttgtgcggcccgtcgctgtcgatctgacttacatccccgtggtcggacacgccctgagcgccagccagttcaacgacactctgaacgtgtacctgactgcccacaacgcccttggaagcagcctgcacactgccggagtggacctcgccaagttctttgatccccttacccgcggggtgttgaccaggactccgctcatgtcccaactgatcttcatcatcgaccccaccattagcgccattgacggcctctacgaccttctcggtattggtatcccaaatcagggtggcatcctgtactcctcactggaatacttcgaaaaggccctggaggagctcgccgccgaacaagtcggtggccagtcgcagctgggaaagctgaacccagatgtgaacctggtggacaccctgaacggaggagagtacactgtgtttgccccaaccaacgccgcgttcgacaagctccccgcggcaaccatcgaccagctgaaaactgatgccaagctgctctcctccattctgacctaccatgtcatcgcgggacaagcatccccctcccgaatcgacgggactcaccagacccttcggcggactgcgccagcacccccgtgggcgaagatgatggcagtggtcggaggcgccctggcttacctggtggtcaagaccttgatcaatgccacccagctgctcaagctgctggccaagctgggagtgtccaccgcgaacgccaccgtgtatatgattgattcggtgctgatgcagcctttcttcgacccgagcgcgtcattcccctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 220 - protein, 132 aa, Mycobacterium tuberculosis
TAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 221 - protein, 18 aa, Mycobacterium tuberculosis
TVPPPVVAANRAELAVLA

SEQ ID NO: 222 - protein, 18 aa, Mycobacterium tuberculosis
MVDPVVVAANRSAFVQLV

SEQ ID NO: 223 - protein, 1109 aa, synthetic construct
MRVTAPRTLILLLSGALALTETWATAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGPGPGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSFVMAYPEMLAAAADTLQSIGATTVASNAAAAAPTTGVVPPAADEVSALTAAHFAAHAAMYQSVSARAAAIHDQFVATLASSASSYAATEVANAAAASGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGTVPPPVVAANRAELAVLAGPGPGMVDPVVVAANRSAFVQLVGIVAGLAVLAVVVIGAVVAAVMCRRKSSGGKGGSYSQAACSDSAQGSDVSLTA

SEQ ID NO: 224 - RNA, 3511 nt, synthetic construct
  source 1..3511: mol_type = other RNA
acatttgcttctgacacaactgtgttcactagcaacctcaaacagccaccatgcgcgtgactgcgcctaggaccctgattctgctcctgtcgggtgccctcgctctgaccgaaacctgggccaccgccgcgagcgacaattttcagttaagccagggtggccagggatttgccatcccgatcggacaggccatggctatcgcgggccagatcagatccggaggcggatccccgactgtacacatcggtccgaccgccttcttgggactgggagtggtggacaacaacgggaatggcgcacgggtgcagagggtcgtgggctcggcaccagccgcctctctgggaatcagcaccggggacgttatcaccgctgtggatggtgcccccatcaattccgccacggcgatggccgatgccctgaacggccatcaccccggggatgtgatatccgtgacctggcagaccaagtccggcggtacaaggaccggcaacgtcactctggccgagggtcctcctgcgggccctggtcctggtgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggacctgggcccggcgtggacttcggagccctgccacctgaaatcaactccgcccggatgtacgcaggacctggatccgccagcctggtggcggccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcggccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcgggccggggcctggatcattcgtgatggcctaccctgagatgctggcagccgcggccgacaccctgcaatccattggcgccactaccgtggcatcgaacgcggcagccgccgcaccgactaccggcgtggtgccgcccgccgctgatgaagtgtccgcccttactgccgcccacttcgccgcccacgccgcgatgtaccagtccgtgtccgcgcgcgccgccgctatccacgatcagttcgtcgccacactggccagctcagcctcctcatacgccgctactgaagtggcaaacgcagccgctgcatccggcccgggacccggaacgatcaactaccagtttggcgacgtggacgcgcacggggcgatgatcagggctcaagcggggtccctcgaggccgaacaccaagcgattatctcggatgtcctcaccgcgtccgacttctggggtggagccggaagcgccgcctgccaaggtttcatcacccaactgggtcgcaactttcaagtcatctacgaacaggccaacgcccacggacagaaggtccaagccgccggaaacaacatggcacagaccgactccgcggtcggatcgagctgggccggacccgggccaggtcccgtgggcggacagagctcgttctattctgactggtactcacccgcatgtggaaaggccgggtgccagacctacaaatgggaaaccttcttgacttccgagctgcctcagtggttgtccgccaatcgggccgtgaagccgaccggctcggcggccatcggcctctccatggccggctcgtccgcgatgattcttgccgcctaccacccgcagcagttcatctacgccgggtccctctcggcactgctggaccccagtcagggaatgggcggacccggaccgggaactgtcccgcccccggtggtggctgctaaccgcgccgaacttgccgtgctggccggtccagggccgggaatggtggacccagtggtggtggcagcgaacagatccgcattcgtgcaactggtcggcattgtggccggactggccgtgttggccgtcgtcgtgatcggcgctgtggtcgccgccgtgatgtgccgaagaaagtccagcggtggaaaggggggatcctactcacaagcagcctgcagcgactcagcgcaggggtcggacgtgtcactgacagcagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 225 - protein, 1095 aa, synthetic construct
MAAPGSARRPLLLLLLLLLLGLMHCASATAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGPGPGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSFVMAYPEMLAAAADTLQSIGATTVASNAAAAAPTTGVVPPAADEVSALTAAHFAAHAAMYQSVSARAAAIHDQFVATLASSASSYAATEVANAAAASGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGTVPPPVVAANRAELAVLAGPGPGMVDPVVVAANRSAFVQLVLIPIAVGGALAGLVLIVLIAYLVGRKRSHAGYQTI

SEQ ID NO: 226 - RNA, 3478 nt, synthetic construct
  source 1..3478: mol_type = other RNA
acatttgcttctgacacaactgtgttcactagcaacctcaaacagccaccatggcggctccgggaagcgcacgacgcccgctgctgctcttgttgctgctcctcctgctgggtctgatgcactgcgcctcggccaccgccgcgagcgacaattttcagttaagccagggtggccagggatttgccatcccgatcggacaggccatggctatcgcgggccagatcagatccggaggcggatccccgactgtacacatcggtccgaccgccttcttgggactgggagtggtggacaacaacgggaatggcgcacgggtgcagagggtcgtgggctcggcaccagccgcctctctgggaatcagcaccggggacgttatcaccgctgtggatggtgcccccatcaattccgccacggcgatggccgatgccctgaacggccatcaccccggggatgtgatatccgtgacctggcagaccaagtccggcggtacaaggaccggcaacgtcactctggccgagggtcctcctgcgggccctggtcctggtgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggacctgggcccggcgtggacttcggagccctgccacctgaaatcaactccgcccggatgtacgcaggacctggatccgccagcctggtggcggccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcggccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcgggccggggcctggatcattcgtgatggcctaccctgagatgctggcagccgcggccgacaccctgcaatccattggcgccactaccgtggcatcgaacgcggcagccgccgcaccgactaccggcgtggtgccgcccgccgctgatgaagtgtccgcccttactgccgcccacttcgccgcccacgccgcgatgtaccagtccgtgtccgcgcgcgccgccgctatccacgatcagttcgtcgccacactggccagctcagcctcctcatacgccgctactgaagtggcaaacgcagccgctgcatccggcccgggacccggaacgatcaactaccagtttggcgacgtggacgcgcacggggcgatgatcagggctcaagcggggtccctcgaggccgaacaccaagcgattatctcggatgtcctcaccgcgtccgacttctggggtggagccggaagcgccgcctgccaaggtttcatcacccaactgggtcgcaactttcaagtcatctacgaacaggccaacgcccacggacagaaggtccaagccgccggaaacaacatggcacagaccgactccgcggtcggatcgagctgggccggacccgggccaggtcccgtgggcggacagagctcgttctattctgactggtactcacccgcatgtggaaaggccgggtgccagacctacaaatgggaaaccttcttgacttccgagctgcctcagtggttgtccgccaatcgggccgtgaagccgaccggctcggcggccatcggcctctccatggccggctcgtccgcgatgattcttgccgcctaccacccgcagcagttcatctacgccgggtccctctcggcactgctggaccccagtcagggaatgggcggacccggaccgggaactgtcccgcccccggtggtggctgctaaccgcgccgaacttgccgtgctggccggtccagggccgggaatggtggacccagtggtggtggcagcgaacagatccgcattcgtgcaactggtcctgattcctattgcggtcggcggggctctcgccgggctcgtgctcattgtcctgattgcctacctggtcggacgcaagaggtcccatgcgggctaccagaccatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 227 - RNA, 720 nt, synthetic construct
  source 1..720: mol_type = other RNA
atggtgagcaagggcgaggaggacaacatggccatcatcaaggagttcatgcggttcaaggtgcacatggagggcagcgtgaacggccacgagttcgagatcgagggcgagggcgagggccggccctacgagggcacccagaccgccaagctgaaggtgaccaagggcggccccctgcccttcgcctgggacatcctgagcccccagttcatgtacggcagcaaggcctacgtgaagcaccccgccgacatccccgactacctgaagctgagcttccccgagggcttcaagtgggagcgggtgatgaacttcgaggacggcggcgtggtgaccgtgacccaggacagcagcctgcaggacggcgagttcatctacaaggtgaagctgcggggcaccaacttccccagcgacggccccgtgatgcagaagaagaccatgggctgggaggccagcagcgagcggatgtaccccgaggacggcgccctgaagggcgagatcaagcagcggctgaagctgaaggacggcggccactacgacgccgaggtgaagaccacctacaaggccaagaagcccgtgcagctgcccggcgcctacaacgtgaacatcaagctggacatcaccagccacaacgaggactacaccatcgtggagcagtacgagcgggccgagggccggcacagcaccggcggcatggacgagctgtacaagagcggcaactga

SEQ ID NO: 228 - protein, 5 aa, synthetic construct
GPGPG

SEQ ID NO: 229 - protein, 96 aa, Mycobacterium tuberculosis; identical to SEQ ID NO: 57

SEQ ID NO: 230 - protein, 325 aa, Mycobacterium tuberculosis; identical to SEQ ID NO: 64

SEQ ID NO: 231 - protein, 391 aa, Mycobacterium tuberculosis; identical to SEQ ID NO: 61

SEQ ID NO: 232 - protein, 94 aa, Mycobacterium tuberculosis; identical to SEQ ID NO: 79

SEQ ID NO: 233 - protein, 98 aa, Mycobacterium tuberculosis; identical to SEQ ID NO: 80

SEQ ID NO: 234 - protein, 100 aa, Mycobacterium tuberculosis; identical to SEQ ID NO: 83

SEQ ID NO: 235 - protein, 95 aa, Mycobacterium tuberculosis; identical to SEQ ID NO: 84

SEQ ID NO: 236 - protein, 99 aa, Mycobacterium tuberculosis; identical to SEQ ID NO: 63

SEQ ID NO: 237 - protein, 338 aa, Mycobacterium tuberculosis; identical to SEQ ID NO: 81

SEQ ID NO: 238 - protein, 539 aa, Mycobacterium tuberculosis; identical to SEQ ID NO: 62

SEQ ID NO: 239 - protein, 97 aa, Mycobacterium tuberculosis; identical to SEQ ID NO: 73

SEQ ID NO: 240 - protein, 415 aa, Mycobacterium tuberculosis; identical to SEQ ID NO: 58

SEQ ID NO: 241 - protein, 101 aa, Mycobacterium tuberculosis
MHVSFVMAYPEMLAAAADTLQSIGATTVASNAAAAAPTTGVVPPAADEVSALTAAHFAAHAAMYQSVSARAAAIHDQFVATLASSASSYAATEVANAAAAS

SEQ ID NO: 242 - protein, 144 aa, Mycobacterium tuberculosis; identical to SEQ ID NO: 68

SEQ ID NO: 243 - protein, 405 aa, Mycobacterium tuberculosis; identical to SEQ ID NO: 76

SEQ ID NO: 244 - protein, 6 aa, unidentified
VVAANR

SEQ ID NO: 245 - protein, 4 aa, unidentified
AANR

SEQ ID NO: 246 - protein, 10 aa, unidentified
GAPINSATAM

SEQ ID NO: 247 - protein, 4 aa, unidentified
AENR

(19) United States
(12) Patent Application Publication
(10) Pub. No.: US 2026/0306603 A1
(43) Pub. Date: Oct. 8, 2026

(54) LIPID NANOPARTICLES FOR THE PREVENTION OF TUBERCULOSIS OR OTHER MYCOBACTERIAL INFECTIONS

(71) Applicant: Akagera Medicines, Inc., Boxford, MA (US)

(72) Inventors: Ross Fulton, Southborough, MA (US); Daryl C. Drummond, Lincoln, MA (US); Robin Friedman, Medford, MA (US); Christian Cobaugh, Stowe, VT (US); Mark E. Hayes, Mill Valley, CA (US)

(73) Assignee: Akagera Medicines, Inc., Boxford, MA (US)

(21) Appl. No.: 19/141,236

(22) PCT Filed: Dec. 22, 2023
(86) PCT No.: PCT/US2023/085680

§ 371 Date: Jun. 19, 2025

Related U.S. Application Data
(60) Provisional application No. 63/476,916, filed on Dec. 22, 2022.

(51) Int. Cl. A61K 39/04; A61K 9/1272; A61K 39/00; A61P 37/04

(52) U.S. Cl. CPC A61K 39/04; A61K 9/1272; A61P 37/04; A61K 2039/53; A61K 2039/605

(57) ABSTRACT

Aspects of the present disclosure provides for improved Mycobacterium tuberculosis vaccine compositions of ionizable lipid nanoparticles for the delivery of immunogenic nucleic acids to cells. Anionic phospholipids, including phosphatidyl serine and phosphatidylglycerol are included in the lipid nanoparticles to increase the transfection efficiency in dendritic cells. In some embodiments, the incorporation of mono-unsaturated alkyl chain analogs in dimethylaminopropyl-dioxolane or heterocyclic ketal ionizable lipids in the formulation provided high levels of transfection in human dendritic cells, compared to other ionizable lipids in the same family, and demonstrated good stability to oxidative damage. Other aspects of the present disclosure provide mRNA that encodes for concatenated peptides encoding for multiple MHC-II tuberculosis epitopes, and optionally includes a second mRNA encoding for concatenated MHC-I tuberculosis epitopes.

### RELATED APPLICATIONS

This patent application claims the benefit of and priority to U.S. Provisional Patent Application No. 63/476,916, filed Dec. 22, 2022, which is incorporated herein by reference in its entirety.

### REFERENCE TO SEQUENCE LISTING

This specification includes a sequence listing submitted herewith, which includes the file entitled 191016-010702_PCT_SL.xml having the following size: 324,933 bytes which was created Dec. 21, 2023, the contents of which are incorporated by reference herein.

### FIELD

Aspects of present disclosure relates to dendritic-cell targeted lipid nanoparticles (LNP) incorporating mRNA encoding for combinations of specific CD8 and CD4 T-cell epitopes found in Mycobacterium tuberculosis. In some embodiments, a LNP comprising one or more cationic ionizable lipid(s) is useful for delivery of mRNA, for dendritic cell targeting or methods of using these LNP compositions as a vaccine for the prevention of tuberculosis or other mycobacterial infections.

### BACKGROUND

Lipid nanoparticles (LNP) are used for the delivery of therapeutic nucleic acids to cells. For example, LNP pharmaceutical compositions are employed in vaccines to deliver mRNA therapeutics. LNP formulations typically include an ionizable cationic lipid (ICL). However, it is known in the art that certain ICL compounds are undesirably sensitive to oxidation during storage. Therefore, there is a need for improved ICL compounds with improved stability to oxidative degradation while in storage, while also providing desired transfection activity or potency in cells when incorporated in a LNP with a therapeutic agent such as a nucleic acid.

LNP compositions, including stable nucleic acid lipid particle (SNALP) compositions, are useful for delivery of nucleic acid therapies for various infectious diseases. Infectious diseases such as tuberculosis, HIV/AIDS, malaria, and COVID-19 represent significant challenges to human health. Mycobacteria, for example, is a genus of bacteria responsible for tuberculosis (TB). According to the World Health Organization, worldwide, TB is one of the top 10 causes of death and the leading cause of death from a single infectious agent. Despite current best efforts, there have been significant challenges in the development of effective vaccines for the prevention of many infectious diseases. New efforts in the identification of individual or combinations of antigenic peptides has helped improved the efficiency of vaccines. Nonetheless, significant opportunities remain in the engineering of adjuvants to help efficiently deliver and present these antigenic sequences to professional antigen presenting cells, like dendritic cells. mRNA coding for antigenic peptides or proteins combined with ionizable cationic lipid nanoparticles represent a particularly promising strategy in the development of a vaccine. There is a need for safe and effective therapies comprising LNP pharmaceutical compositions for delivery of mRNA for treatment and prevention of various diseases, including vaccine compositions.

### SUMMARY

Lipid nanoparticle (LNP) compositions are provided herein, and methods of making and using the same. In some embodiments, the LNP compositions comprise a nucleic acid such as messenger ribonucleic acid (mRNA). In some embodiments, the LNP compositions are vaccines, including LNP formulations comprising mRNA that encodes an immune system epitope, or an antigen recognized by the immune system.

In some aspects, the LNP comprises nucleic acid containing a chemically modified mRNA, wherein the chemically modified mRNA comprises N1-methylpseudouridine. In some aspects, the LNP comprises nucleic acid comprising a 5′ untranslated region (UTR) and 3′ UTR, polyA tail of about 80 to about 140 nucleotides in length, and (i) a 5′ enzymatic or (ii) a 5′ clean cap. In some aspects, the LNP comprises a chemically modified mRNA, wherein the chemically modified mRNA comprises N1-methylpseudouridine

In some embodiments, the LNP composition comprises: (a) a nucleic acid; (b) an ionizable cationic lipid comprising a KC3 ionizable cationic lipid at a N/P ratio of 3 to 7 relative to the nucleic acid, the ionizable cationic lipid present in the LNP composition in a total amount of 46-54 mol % of a total lipid content of the LNP composition; (c) one or more phospholipids in a total amount of 5-20 mol % of the total lipid content of the LNP composition; (d) one or more anionic phospholipids in a total amount of 2-8 mol % of the total lipid content of the LNP composition; (e) a conjugated lipid in a total amount of 1-3.5 mol % of the total lipid content of the LNP composition; and (f) a sterol such cholesterol (e.g., in an amount providing the remainder of the LNP composition). In some aspects, the one or more anionic phospholipids is a phosphatidylserine (PS) or phosphatidylglycerol (PG). In some aspects, the one or more anionic phospholipids is selected from the group consisting of: dipalmitoylphosphatidyl-L-serine (DPPS), or distearoylphosphatidyl-L-serine (DSPS), distearoylphosphatidylglycerol (DSPG), and dipalmitoyphosphatidylglycerol (DPPG). In some aspects, the one or more phospholipids comprises distearoylphosphatidylcholine (DSPC), hydrogenated soy phosphatidylcholine (HSPC), dipalmitoylphosphatidylcholine (DPPC) or a combination thereof. In some aspects, the conjugated lipid is PEG (2000)-dimyristoylglycerol (PEG-DMG). In some aspects, the sterol is cholesterol. In some aspects, the ionizable cationic lipid comprises 3-((S)-2,2-di((Z)-octadec-9-en-1-yl)-1,3-dioxolan-4-yl)-N,N-dimethylpropan-1-amine (KC3-OA). In some aspects, the ionizable cationic lipid further comprises a KC4 ionizable cationic lipid, such as 4-rac-2,2-di((Z)-octadec-9-en-1-yl)-1,3-dioxolan-4-yl)-N,N-dimethylbutan-1-amine (AKG-KC4-OA). In some aspects, the LNP composition consists of: 48 mol % KC3-OA; 5 mol % DPPS or DSPG; 5-10 mol % DSPC or HSPC; 1.5 mol % PEG-DMG; and cholesterol. In some aspects, the LNP composition consists of: 48 mol % KC3-OA; 5 mol % DPPS or DSPG; 5 mol % DSPC or HSPC; 1.5 mol % PEG-DMG; and 40.5 mol % cholesterol. In some aspects, the LNP composition consists of: 48 mol % KC3-OA; 5 mol % DPPS or DSPG; 10 mol % DSPC or HSPC; 1.5 mol % PEG-DMG; and 35.5 mol % cholesterol.

In some embodiments, a method of eliciting a T cell response in a host is provided, comprising administering to the host a nucleic acid sequence disclosed herein or a nucleic acid having at least 90% sequence identity or complementarity to a sequence disclosed herein, and/or a sequence encoding a T cell epitope from Mycobacterium tuberculosis (Mtb), or a polynucleotide sequence having at least 90% identity or complementarity to a sequence disclosed herein and/or a polynucleotide sequence of a Mtb antigen recognized by T cells.

A lipid nanoparticle (LNP) composition consisting of: a messenger ribonucleic acid (mRNA) encoding one or more Mycobacterium tuberculosis (Mtb) proteins selected from the group consisting of CFP10/Rv3874, ESAT-6/Rv3875, Mtb32A/Rv0125, Mtb39A/Rv1196, Ag85B/Rv1886c, EsxW/Rv3620c, Esx V/Rv3619c, PE13/Rv1195, PPE30/Rv1802, PPE40/Rv2356c and TB10.4/Rv0288; an ionizable cationic lipid comprising a KC3 ionizable cationic lipid at a N/P ratio of 4 to 6 relative to the mRNA, the ionizable cationic lipid present in the LNP composition in a total amount of 46-54 mol % of a total lipid content of the LNP composition; one or more phospholipids selected from the group consisting of distearoylphosphatidylcholine (DSPC), hydrogenated soy phosphatidylcholine (HSPC), and dipalmitoylphosphatidylcholine (DPPC), in a total amount of 10-18 mol % of the total lipid content of the LNP composition; one or more anionic phospholipids selected from the group consisting of dipalmitoylphosphatidyl-L-serine (DPPS), or distearoylphosphatidyl-L-serine (DSPS), distearoylphosphatidylglycerol (DSPG), and dipalmitoyphosphatidylglycerol (DPPG) in a total amount of 2-8 mol % of the total lipid content of the LNP composition; PEG (2000)-dimyristoylglycerol (PEG-DMG) in a total amount of 1-3.5 mol % of the total lipid content of the LNP composition; and cholesterol (e.g., 35.5-40.5 mol % cholesterol).

Aspects of the disclosure relate to a lipid nanoparticle (LNP) composition comprising a KC3 ionizable cationic lipid, cholesterol, one or more phospholipids comprising at least one anionic phospholipid, a conjugated lipid and one or more nucleic acid sequence encoding a T cell epitope from Mycobacterium tuberculosis (Mtb).

In some embodiments, the LNP composition comprises 48 mol % of the KC3 ionizable cationic lipid, 40.5 mol % cholesterol, 5 mol % (L-Serine) DPPS lipid, 5 mol % DSPC or DPPC; and a total of 10 mol % phospholipid concentration, wherein each mol % refers to the mol % of the total lipid content of the LNP composition.

In some embodiments, the LNP composition comprises 48 mol % of the KC3 ionizable cationic lipid, 40.5 mol % cholesterol, and 5 mol % (L-Serine) DPPS lipid, wherein each mol % refers to the mol % of the total lipid content of the LNP composition. In some embodiments, the LNP composition comprises 48 mol % of the KC3 ionizable cationic lipid, 38.5 mol % cholesterol, and 5 mol % (L-Serine) DPPS lipid, wherein each mol % refers to the mol % of the total lipid content of the LNP composition. In some embodiments, the LNP composition comprises 45 mol % of the KC3 ionizable cationic lipid, 42.7 mol % cholesterol, and 5 mol % (L-Serine) DPPS lipid, wherein each mol % refers to the mol % of the total lipid content of the LNP composition. In some embodiments, the LNP composition comprises 50 mol % of the KC3 ionizable cationic lipid, 38.5 mol % cholesterol, 5 mol % (L-Serine) DPPS lipid, and a total of 10 mol % phospholipid concentration, wherein each mol % refers to the mol % of the total lipid content of the LNP composition. In some embodiments, the LNP composition comprises 48 mol % of the KC3 ionizable cationic lipid, 40.5 mol % cholesterol, 5 mol % (L-Serine) DPPS lipid, and a total of 10 mol % phospholipid concentration, wherein each mol % refers to the mol % of the total lipid content of the LNP composition. In some embodiments, the LNP composition comprises 48 mol % of the KC3 ionizable cationic lipid, 40.5 mol % cholesterol, 5 mol % (L-Serine) DPPS lipid, 5 mol % DSPC or DPPC; and a total of 10 mol % phospholipid concentration, wherein each mol % refers to the mol % of the total lipid content of the LNP composition. In some embodiments, the LNP composition comprises 46.5 mol % of the KC3 ionizable cationic lipid, 42 mol % cholesterol, 5 mol % (L-Serine) DPPS lipid, wherein each mol % refers to the mol % of the total lipid content of the LNP composition. In some embodiments, the LNP composition comprises 15 mol % total phospholipid and 35.5 mol % cholesterol. In some embodiments, the LNP composition comprises 10 mol % total phospholipid and 40.5 mol % cholesterol. In some embodiments, the LNP composition comprises 40.5 mol % cholesterol, 5% anionic lipid (DPPS) and 5% PC (DSPC or DPPC) and a total of 10 mol % phospholipid concentration. In some embodiments, the LNP composition comprises 48 mol % cationic ionizable lipid, 5 mol % PC (DPPC), 5 mol % anionic lipid (DPPS), 40.5 mol % cholesterol, 1.5 mol % conjugated lipid (PEG-DMG).

In some aspects, the LNP comprises a nucleic acid sequence (e.g., mRNA) encoding a T cell epitope from Mycobacterium tuberculosis (Mtb), or a Mtb antigen recognized by T cells. In some aspects, the LNP comprises a nucleic acid sequence that is mRNA encoding a concatenated sequence of T-cell epitopes present in Mtb or a Mtb antigen recognized by T Cells. In some aspects, the LNP comprises a nucleic acid sequence that is mRNA encoding one or more Mtb proteins selected from the group consisting of CFP10/Rv3874, ESAT-6/Rv3875, Mtb32A/Rv0125, Mtb39A/Rv1196, and Ag85B/Rv1886c. In some aspects, the LNP comprises a nucleic acid sequence that is mRNA comprising one or more nucleic acid sequences selected from the group consisting of: SEQ ID NO:2, SEQ ID NO:3, SEQ ID NO:6, SEQ ID NO:7, and SEQ ID NO: 220. In some aspects, the LNP comprises a nucleic acid sequence that is mRNA encoding one or more Mtb proteins selected from the group consisting of EsxW/Rv3620c, EsxV/Rv3619c, PE13/Rv1195, PPE30/Rv1802, PPE40/Rv2356c and TB10.4/Rv0288. In some aspects, the LNP comprises a nucleic acid sequence that is mRNA comprising one or more nucleic acid sequences selected from the group consisting of: SEQ ID NO:1, SEQ ID NO:4, SEQ ID NO:5, SEQ ID NO: 31, SEQ ID NO:221, and SEQ ID NO:222. In some aspects, the LNP comprises a nucleic acid sequence that comprises the concatenated nucleic acid-encoded sequence includes an N-terminal and C-terminal signal peptide selected from Sec/MITD, Lamp1, HLA-Dra, or tPA. In some aspects, the LNP comprises a nucleic acid sequence that is an mRNA having a sequence selected from SEQ ID NOs: 34, 36, 38, 40, 42, 44, 224 and 226. In some aspects, the LNP comprises nucleic acid that is an mRNA encoding an amino acid sequence selected from SEQ ID NOs: 33, 35, 37, 39, 41, 43, 86-105, 207-210, 223 and 225.

In some embodiments, the one or more nucleic acids is a mRNA. In some embodiments, the mRNA encodes a concatenated sequence of T-cell epitopes present in Mtb. In some embodiments, the concatenated sequence of T-cell epitopes comprise an amino acid sequence set forth in SEQ ID NOs: 1-17, 106-137, 138-203. In some embodiments, the concatenated sequence of T-cell epitopes comprises an amino acid sequence with at least 90% sequence identity (e.g. 90%, 91%, 92%, 93%, 94%, 95%, 96%, 97%, 98%, 99% or more) with amino acid sequence set forth in SEQ ID NOs: 1-17, 45-85, 106-137, 138-203. In some embodiments, the concatenated nucleotide sequence comprises two or more sequences encoding for peptides or proteins that can elicit MHC class II-restricted CD4 T cell responses.

In some embodiments, the two or more MHC class II epitopes selected from the group: EsxV (Rv3619), EsxW (Rv3620c), EsxB/CFP10 (Rv3874), EsxA/ESAT-6 (Rv3875), AMtb39A (Rv1196), Ag85B (Rv1886c), and EsxH/TB10.4 (Rv0288). In some embodiments, the two or more MHC class II epitopes comprises peptides or proteins from EsxV (Rv3619), EsxW (Rv3620c), EsxB/CFP10 (Rv3874), EsxA/ESAT-6 (Rv3875), AMtb39A (Rv1196), Ag85B (Rv1886c), and EsxH/TB10.4 (Rv0288) (SEQ ID NOs. 1-7).

In some embodiments, the concatenated nucleic acid-encoded sequence includes the seven proteins in and order N-terminal to C-terminal selected from: EsxB/CFP10 (Rv3874), EsxA/ESAT-6 (Rv3875), EsxH/TB10.4 (Rv0288), AAg85B (Rv1886c), AMtb39A (Rv1196), EsxW (Rv3620c), and EsxV (Rv3619), or EsxB/CFP10 (Rv3874), EsxA/ESAT-6 (Rv3875), EsxW (Rv3620c), EsxV (Rv3619), EsxH/TB10.4 (Rv0288), AAg85B (Rv1886c), and AMtb39A (Rv1196), or EsxB/CFP10 (Rv3874), AMtb39A (Rv1196), EsxA/ESAT-6 (Rv3875), EsxW (Rv3620c), EsxH/TB10.4 (Rv0288), EsxV (Rv3619), and AAg85B (Rv1886c). (SEQ ID NOs. 18, 19, and 20)

In some embodiments, the composition comprises a nucleic acid encoding for 5 or more non-overlapping CD4 T cell epitopes in the form of peptides, wherein optionally the peptides are from 12 to 50 amino acids long.

In some embodiments, the concatenated nucleic acid-encoded sequence optionally comprises 10 selected MHC-II epitopes comprising: AQIYQAVSAQAAAIH (SEQ ID NO. 9), PSPSMGRDIKVQFQS (SEQ ID NO. 10), GINTIPIAINEAEYV (SEQ ID NO. 11), AAFQGAHARFVAAAA (SEQ ID NO. 12), AGWLAFFRDLVARGL (SEQ ID NO. 13), ASIIRLVGAVLAEQH (SEQ ID NO. 14), MSFVTTQPEALAAAA (SEQ ID NO. 8), MHVSFVMAYPEMLAA (SEQ ID NO. 15), AYGSFVRTVSLPVGA (SEQ ID NO. 16), and LENDNQLLYNYPGAL (SEQ ID NO. 17).

In some embodiments, the concatenated nucleic acid-encoded sequence includes GPGPG (SEQ ID NO. 228) linker sequences between each of the concatenated epitopes.

In some embodiments, the one or more nucleic acid comprises a nucleic acid sequence set forth in SEQ ID NOs: 34, 36, 38, 40, 42, and 44.

In some embodiments, the one or more nucleic acid comprises a nucleic acid sequence having at least 90% identity, at least 91%, at least 92%, at least 93%, at least 94%, at least 95%, at least 96%, at least 97%, at least 98% or at least 99% with a nucleic acid sequence set forth in SEQ ID NOs: 34, 36, 38, 40, 42, and 44.

In some embodiments, the concatenated nucleic acid-encoded sequence includes an N-terminal and C-terminal signal peptide selected from Sec/MITD, Lamp1, HLA-Dra, or tPA.

In some embodiments, the one or more nucleic acid comprises a chemically modified mRNA, wherein the chemically modified mRNA comprises N1-methylpseudouridine.

In some embodiments, the one or more nucleic acid comprises a 5′ untranslated region (UTR) and 3′ UTR, polyA tail of about 80 to about 140 nucleotides in length, and (i) a 5′ enzymatic or (ii) a 5′ clean cap.

In some embodiments, the one or more nucleic acid is an mRNA having a sequence selected from SEQ ID NOs: 34, 36, 38, 40, 42, 44, 224 and 226.

In some embodiments, the one or more nucleic acid is an mRNA and wherein the amino acid sequence encoded by the mRNA is selected from SEQ ID NOs: 33, 35, 37, 39, 41, 43, 86-105, 207-210, 223 and 225.

In some embodiments, the nucleic acid-encoded concatenated sequence comprises two or more MHC class I epitopes selected from SEQ ID NOs: 106-137 and 138-203.

In some embodiments, the nucleic acid-encoded concatenated sequence includes two or more MHC class I epitopes found in Mycobacterium tuberculosis, depleted of epitopes found in BCG, and selected from SEQ ID NOs: 86-95.

In some embodiments, the nucleic acid-encoded concatenated sequence includes two or more MHC class I epitopes that are ordered to minimize junctional neoepitope generation, and selected from SEQ ID NOs: 86-105.

In some embodiments, the cationic lipid is KC3-OA, KC3-PA, KC3-01, KC3-C17 (8:1), or KC3-C15 (C8:1).

In some embodiments, the LNP comprises the conjugated lipid in a total amount of less than 2 mol % of the total lipid content of the LNP composition.

In some embodiments, the ionizable cationic lipid in a total amount of 45-55 mol % of the total lipid content of the LNP composition; cholesterol is in a total amount of 35-45 mol % of the total lipid content of the LNP composition; the total amount of the one more phospholipid is 7-15 mol % of the total lipid content of the LNP composition; the one or more phospholipids consist of DSPC and the PS lipid is one or more lipids selected from the group consisting of the L-serine configuration of DPPS and DSPS; and the total amount of the PS lipid is about 5 mol % of the total lipid content of the LNP composition.

In some embodiments, the conjugated lipid is PEG-DMG; and the PS lipid is selected from the group consisting of: DSPS (L-isomer) and DPPS.

In some embodiments, the ionizable cationic lipid is KC3-OA.

In some embodiments, the LNP composition has a N/P ratio of 4 to 7. In some embodiments, the LNP composition has a N/P ratio of 5 to 6.

Provided in some aspect of the disclosure is a nucleic acid lipid nanoparticle (LNP) composition comprising: a mRNA having at least 90% identity (e.g. 90%, 91%, 92%, 93%, 94%, 95%, 96%, 97%, 98%, 99% or more) with a nucleic acid sequence set forth in SEQ ID NOs: 34, 36, 38, 40, 42, and 44, ionizable cationic lipid KC3-PA, and a (L-Serine) PS lipid in a total amount of 2.5-10 mol % of the total lipid content of the LNP composition.

In some embodiments, the PS lipid is (L-Serine) DSPS, (L-Serine) DPPS, or a mixture thereof, and the LNP composition further comprises cholesterol and a second phospholipid selected from the group consisting of: DSPC, DOPC, DPPC, HSPC, and SM.

Provided in some aspect of the disclosure isa nucleic acid lipid nanoparticle (LNP) composition comprising: a mRNA having at least 90% identity (e.g. 90%, 91%, 92%, 93%, 94%, 95%, 96%, 97%, 98%, 99% or more) with a nucleic acid sequence set forth in SEQ ID NOs: 34, 36, 38, 40, 42, and 44; a KC3 ionizable cationic lipid in a total amount of 40-65 mol % of the total lipid content of the LNP composition; cholesterol in a total amount of 23.5-43.5 mol % of the total lipid content of the LNP composition; a (L-Serine) PS lipid in a total amount of 2.5-10 mol % of the total lipid content of the LNP composition; DSPC or HSPC phospholipid in a total amount of 5-25 mol % of the total lipid content of the LNP composition; and a PEG-containing conjugated lipid in a total amount of 0.5 mol % to 2.5 mol % of the total lipid content of the LNP composition.

In some embodiments, the ionizable lipid having the chemical structure:

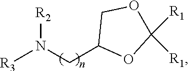

wherein

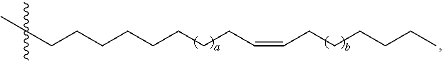

R₁ is
   wherein a is 0 or 1; b is 1, 2, 3 or 4, provided the sum a+b is 1, 2, 3 or 4;
R₂ and R₃ are each independently methyl; and
n is an integer equal to 2 or 3. In some embodiments, n is 3.

In some embodiments, the composition is a vaccine.

Provided in some aspects of the disclosure is a pharmaceutical composition comprising the lipid nanoparticle described herein, and a pharmaceutically acceptable carrier.

Aspects of the disclosure relate to a nucleic acid encoding a concatenated amino acid sequence of T-cell epitopes present in Mycobacterium tuberculosis, the nucleic acid having at least 90% (e.g. 90%, 91%, 92%, 93%, 94%, 95%, 96%, 97%, 98%, 99% or more) identity with a nucleic acid sequence set forth in SEQ ID NOs: 34, 36, 38, 40, 42, and 44.

In some embodiments, ionizable cationic lipids (ICLs) are provided. Cationic lipids are engineered with improved stability to oxidative degradation while in storage, while retaining high transfection activity or potency in cells. Aspects of the disclosure are based in part on the discovery that LNP compositions comprising mRNA and certain ionizable cationic lipids (ICL) enhanced expression of the mRNA in human dendritic cells.

In some embodiments, LNP compositions comprise a targeting ligand directed against cell surface receptors to target lipid nanoparticles in a highly specific manner, including to dendritic cells. In some embodiments, the LNP composition comprises a phosphatidyl-L-serine compound as a targeting ligand, such as dipalmitoylphosphatidyl-L-serine (DPPS), or distearoylphosphatidyl-L-serine (DSPS). In some embodiments, the LNP composition comprises a phosphatidyl-L-serine compound as a targeting ligand and an anionic phospholipid. In some embodiments, the LNP composition comprises a phosphatidylglycerol-containing compound as a targeting ligand such as distearoylphosphatidylglycerol (DSPG) or dipalmitoyphosphatidylglycerol (DPPG), for enhancing expression in human dendritic cells. In some embodiments, LNP compositions comprise both a phosphatidyl-L-serine compound as a targeting ligand, and distearoylphosphatidylcholine (DSPC) as the second phospholipid. In some embodiments, LNP compositions comprise both a phosphatidyl-L-serine compound as a targeting ligand, and distearoylphosphatidylcholine (DSPC) as the second phospholipid without dipalmitoylphosphatidylcholine (DPPC).

Aspects of the disclosure are based in part on the discovery that selection of certain cationic ionizable lipids can enhance the transfection of human dendritic cells. For example, the KC3 cationic ionic lipids were more active in transfecting human dendritic cells in LNP compositions than either the KC2 or diacyl ionizable lipids (UO series). Among the LNP compositions comprising KC3 ionizable cationic lipids, those LNP compositions with ionizable cationic lipids having monounsaturated alkyl chains were unexpectedly both more active and more stable to oxidative degradation than those containing those with the dilinoleyl alkyl chains.

In some embodiments, certain salts of the phosphatidylserine targeting lipids are provided. For example, in some embodiments, the phosphatidylserine targeting lipids can be provided as an ammonium salt of DPPS having improved biophysical properties and higher solubility in the presence of ethanol, a preferred solvent for preparation of LNPs. The sodium salts of DSPS or DSPS were insoluble in ethanol and required both the presence of methanol and heating to allow for their formation, as did the ammonium salt of DSPS. It is contemplated that the other ammonium salts of phosphatidylserine will give rise to the same advantages in solubility and biophysical properties.

In some embodiments, ionizable cationic lipid compositions useful in the preparation of liposomal nanoparticle (LNP) compositions are provided. In some embodiments, liposomal compositions are provided comprising an ionizable cationic lipid having (a) a pair of linear C₁₆ or C₁₈ hydrocarbon chains each comprising a single unsaturated alkenyl double bond within each polyene hydrocarbon chain, covalently bound to a head group comprising a dialkyl amino alkyl group. In some embodiments, the head group of the ionizable cationic lipid has a dialkyl amino group having a pKa of about 6.3-7.5. In some embodiments, the head group of the ionizable cationic lipid comprises a heterocyclyl or alkyl portion covalently bound to the dialkyl amino group. In some embodiments, the head group of the ionizable cationic lipid optionally further comprises a phosphate group. In some embodiments, each lipid tail of the ionizable cationic lipid compound is identical, and each lipid tail has a total of one olefin with a total length of 15, 16, 17 or 18 carbons.

In some embodiments, the LNP compositions comprises a KC3 ionizable cationic lipid. Unless otherwise indicated, the term “KC3 ionizable cationic lipid” as used herein refers to an ionizable cationic lipid having the chemical formula:

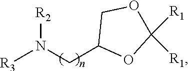

wherein each R₁ is the same or different and is a linear C₁₅ to C₁₉ hydrocarbon chain each comprising one or more unsaturated alkenyl double bond within each polyene hydrocarbon chain; R₂ and R₃ are each independently methyl; and n is 3. In some aspects, each R₁ in the KC3 ionizable cationic lipid is the same and is a linear C₁₆ or C₁₈ hydrocarbon chain each comprising a single unsaturated alkenyl double bond within each polyene hydrocarbon chain.

In some embodiments, the LNP compositions comprises a KC4 ionizable cationic lipid. Unless otherwise indicated, the term “KC4 ionizable cationic lipid” as used herein refers to an ionizable cationic lipid having the chemical formula:

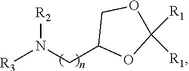

wherein each R₁ is the same or different and is a linear C₁₅ to C₁₉ hydrocarbon chain each comprising one or more unsaturated alkenyl double bond within each polyene hydrocarbon chain; R₂ and R₃ are each independently methyl; and n is 4. In some aspects, each R₁ in the KC4 ionizable cationic lipid is the same and is a linear C₁₆ or C₁₈ hydrocarbon chain each comprising a single unsaturated alkenyl double bond within each polyene hydrocarbon chain.

In some embodiments, the LNP compositions comprises a mixture of a KC3 ionizable cationic lipid and a KC4 ionizable cationic lipid.

In some embodiments, a lipid nanoparticle (LNP) composition comprises an ionizable cationic lipid having the chemical structure of Formula (I):

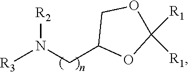

wherein
R₁ is

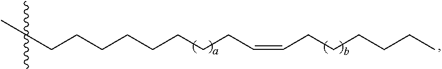

wherein a is 0 or 1; b is 1, 2, 3 or 4, provided the sum a+b is 1, 2, 3 or 4;
R₂ and R₃ are each independently (C₁-C₄)alkyl optionally substituted with hydroxyl; and
n is an integer equal to 2, 3 or 4.

In some embodiments, ionizable cationic lipid compositions are provided. In some embodiments, a lipid nanoparticle (LNP) composition comprises an ionizable cationic lipid having the chemical structure of Formula (I) wherein a is 0 or 1; b is 1, 2, 3 or 4, provided the sum a+b is 1, 2, 3 or 4; R₂ and R₃ are each methyl; and n is an integer equal to 3.

In some embodiments, ionizable cationic lipid compositions are provided. In some embodiments, a lipid nanoparticle (LNP) composition comprises an ionizable cationic lipid having the chemical structure of Formula (I-A):

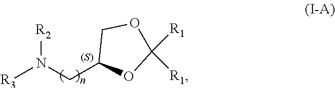

wherein
R₁ is

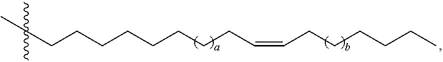

wherein a is 0 or 1; b is 1, 2, 3 or 4, provided the sum a+b is 1, 2, 3 or 4;
R₂ and R₃ are each methyl; and
n is an integer equal to 3.

In some embodiments, a LNP composition comprises an ionizable cationic lipid comprises a pair of identical, lipid hydrocarbon tails having a total of 15, 16, 17 or 18 carbons and comprising a single olefin group, or a pair of olefin groups. In some embodiments, a LNP composition comprises an ionizable cationic lipid selected from the group consisting of:

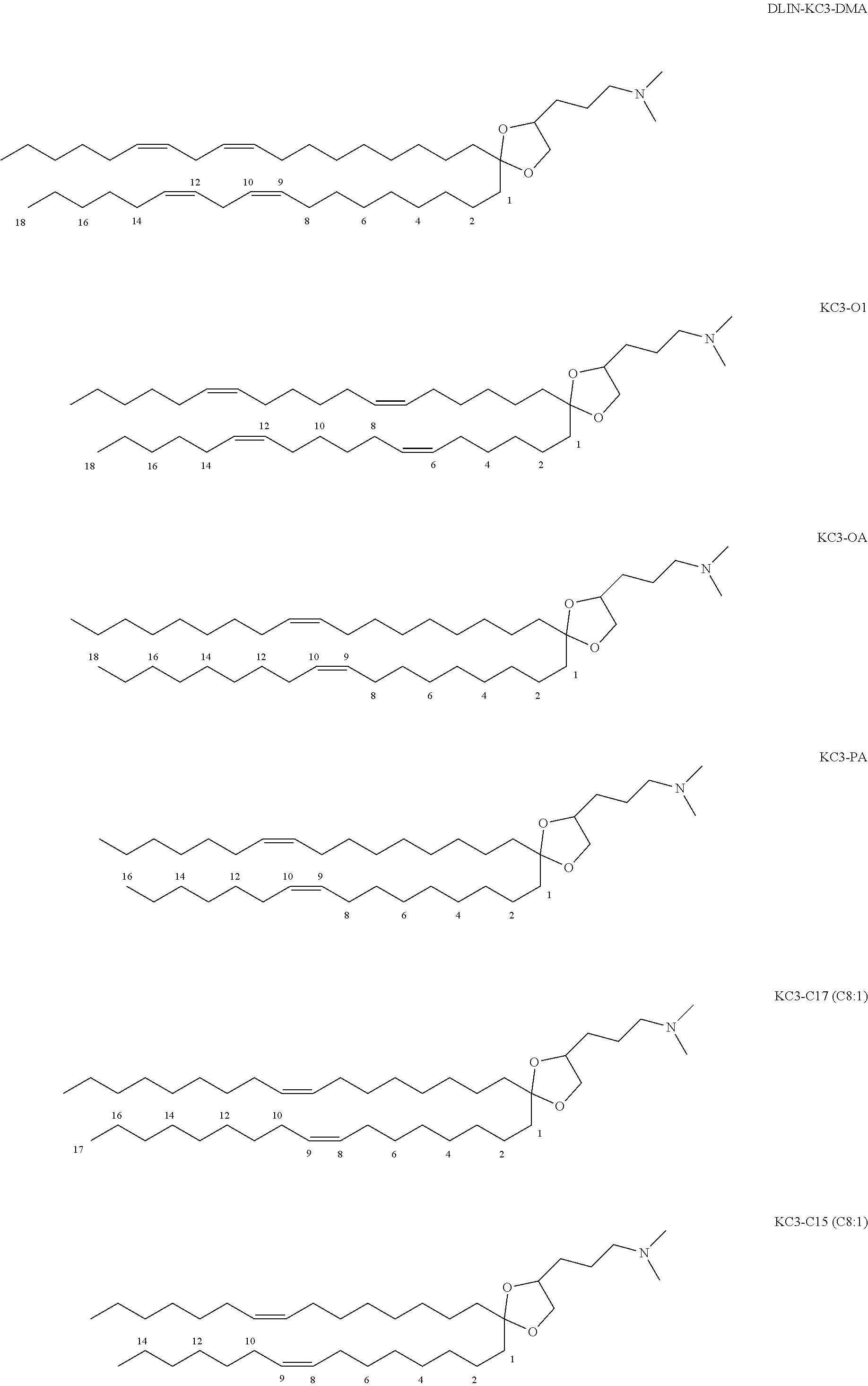

In some embodiments, the ionizable cationic lipid is KC3-PA. In some embodiments, the ionizable cationic lipid is KC3-C15 (C8:1). In some embodiments, the ionizable cationic lipid is KC3-C16 (C8:1). In some embodiments, the ionizable cationic lipid is KC3-C17 (C8:1). In some embodiments, the ionizable cationic lipid is KC3-C18 (C8:1).

In some embodiments, the ionizable cationic lipid is KC3-15. In some embodiments, the ionizable cationic lipid is KC3-16. In some embodiments, the ionizable cationic lipid is KC3-17. In some embodiments, the ionizable cationic lipid is KC3-18.

The salt form of the targeting lipid can influence it's solubility in alcohol containing solvents used in the preparation of lipid nanoparticles. In some embodiments, ionizable cationic lipid compositions are provided. In some embodiments, a lipid nanoparticle (LNP) composition comprises a nucleic acid; an ionizable lipid disclosed herein; a sterol; one or more phospholipids comprising a phosphatidylserine (PS) lipid; and optionally further comprising a conjugated lipid. In some embodiments, a lipid nanoparticle (LNP) composition comprises a mRNA nucleic acid; an ionizable lipid disclosed herein; cholesterol; one or more phospholipids selected from the group consisting of: DSPC, DPPC and DOPC; and a PS lipid selected from the group consisting of: DPPS, DSPS and DOPS; and optionally further comprising a conjugated lipid comprising PEG.

In some embodiments, a LNP composition can comprise an anionic phospholipid. In some embodiments, a LNP composition is prepared using a sodium or ammonium salt of an anionic phospholipid. In some embodiments, the anionic phospholipid salt is a compound of Formula (V-A-1), having the chemical structure:

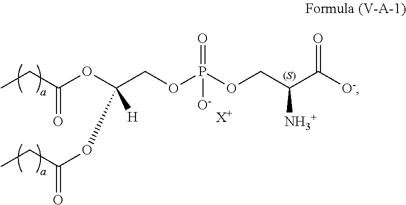

wherein
X⁺ is an ammonium (NH₄⁺) or sodium (Na⁺) cation; and
a is 14, 15 or 16.

In some embodiments, the anionic phospholipid salt is selected from the group consisting of:

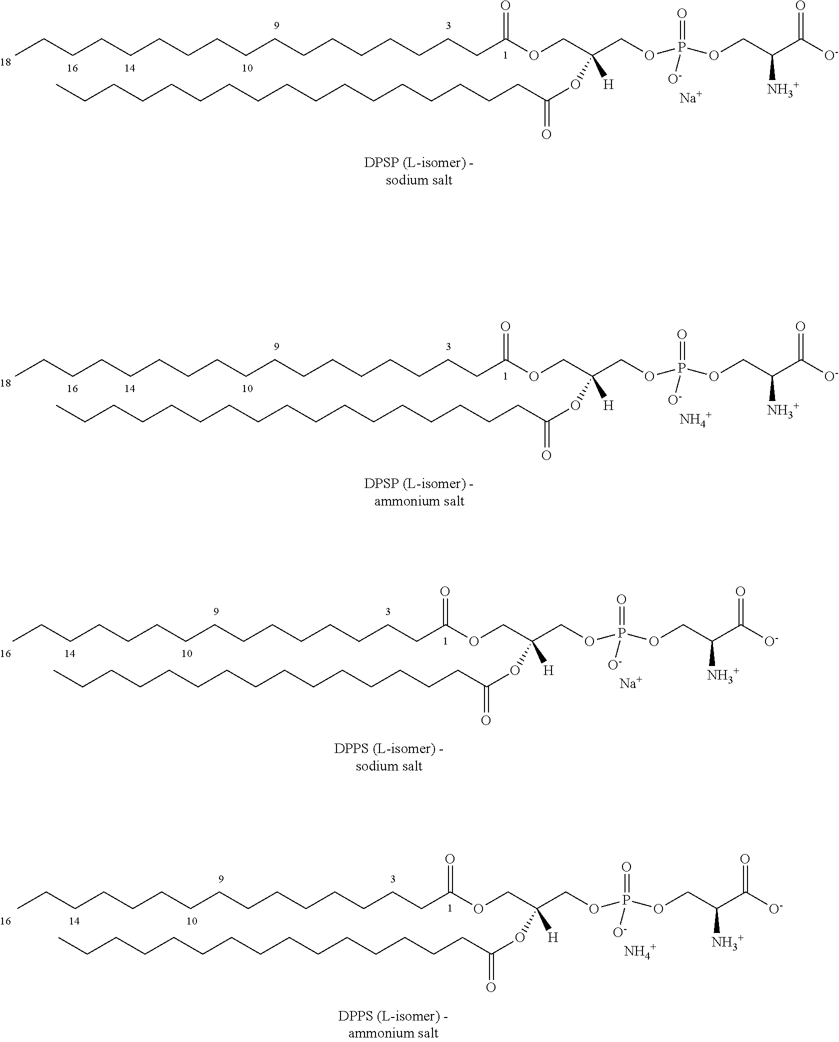

In some embodiments, the anionic phospholipid salt is DSPS (L-isomer) sodium salt. In some embodiments, the anionic phospholipid salt is DSPS (L-isomer) ammonium salt. In some embodiments, the anionic phospholipid salt is DPPS (L-isomer) sodium salt. In some embodiments, the anionic phospholipid salt is DPPS (L-isomer) ammonium salt. In some embodiments the targeting lipid is a sodium or ammonium salt of dipalmitoylphosphatidyl-L-serine (DPPS) or distearoylphosphatidyl-L-serine (DSPS). In some embodiments the targeting lipid is a sodium or ammonium salt of dipalmitoylphosphatidyl-L-serine (DPPS) or distearoylphosphatidyl-L-serine (DSPS).

In some embodiments, a LNP composition can comprise an anionic phospholipid selected from the group consisting of:

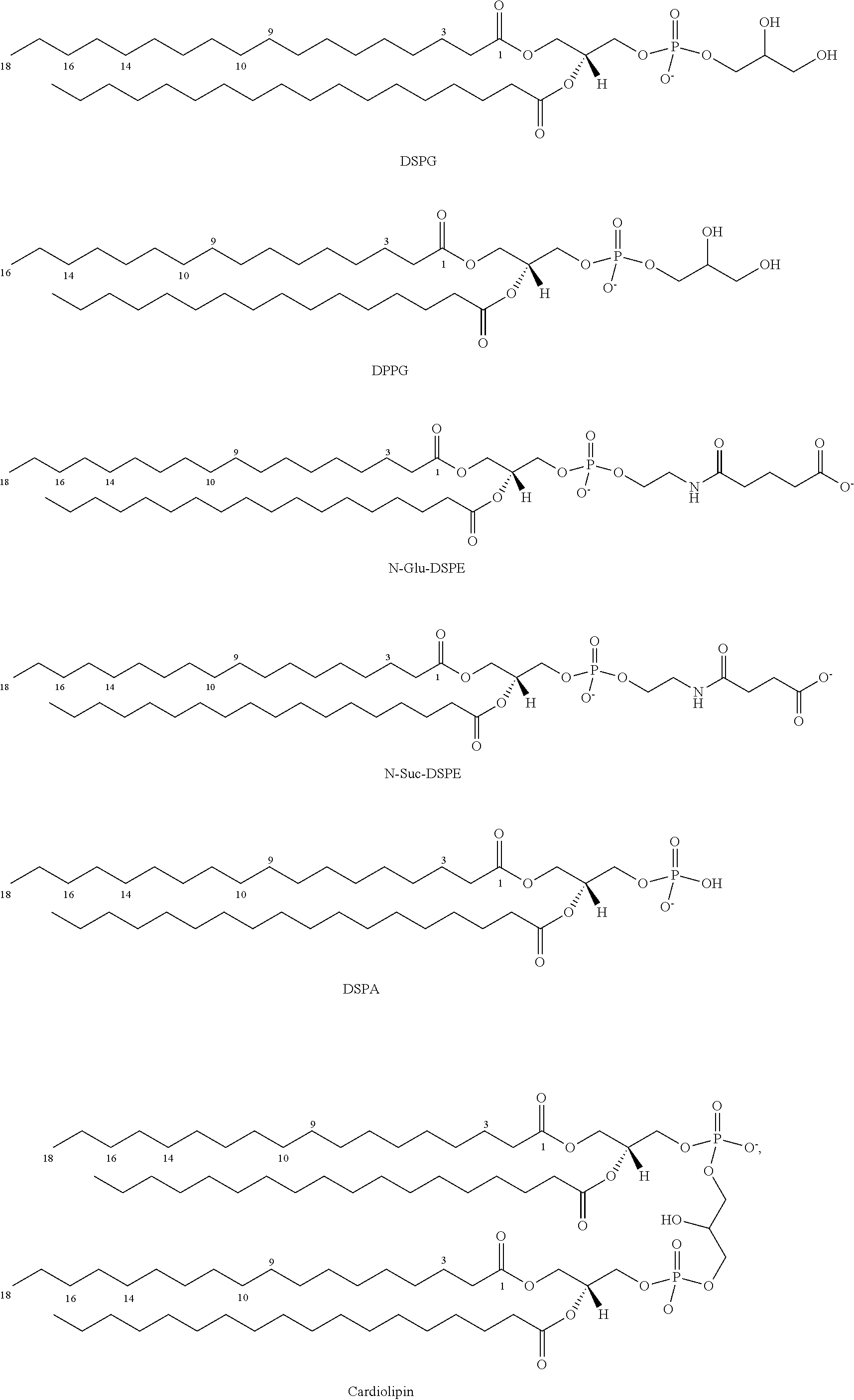

or an ammonium or sodium salt thereof.

In some embodiments, the salt form of phosphatidylserine is highly soluble in ethanol. In some embodiments it is soluble at greater than 0.5 mg/ml, greater than 1 mg/mL, greater than 5 mg/mL, greater than 10 mg/mL, or greater than 20 mg/mL. In some embodiments, the salt is an ammonium salt. In some embodiments, the salt is ammonium itself, an alkylammonium, a dialkylammonium, or a trialkylammonium salt. In some embodiments, the amine is chosen from ammonia, dimethylamine, diethylamine, triethylamine, trimethylamine, 2-(dimethyamino)ethanol, diethanolamine, 2-(diethyamino)ethanol, ethanolamine, ethylenediamine, N-methyl-glucamine, imidazole, histidine, lysine, arginine, 4-(2-hydroxyethyl)-morpholine, piperazine, 1-(2-hydroxyethyl)-pyrrolidine, triethanolamine, and tromethamine (tris(hydroxymethyl)aminomethane), In some embodiments, this targeting lipid is an ammonium salt of DPPS.

Anionic phospholipids, separate from phosphatidyl-L-serine, were also considered as targeting lipids for LNPs. These include phosphatidylglycerol (PG), phosphatidic acid (PA), N-glutaryl-phosphatidylethanolamine (N-Glu-PE), N-succinyl-phosphatidylethanolamine (N-Suc-PE), and cardiolipin. In some embodiments, a LNP comprises anionic phospholipids, separate from phosphatidyl-L-serine, useful as targeting lipids for LNPs. In some embodiments, a LNP comprises anionic phospholipids selected from the group consisting of: phosphatidylglycerol (PG), phosphatidic acid (PA), N-glutaryl-phosphatidylethanolamine (N-Glu-PE), N-succinyl-phosphatidylethanolamine (N-Suc-PE), and cardiolipin. Distearoylphosphatidylglycerol (DSPG), dipalmitoyphosphatidylglycerol (DPPG), N-succinyl-distearoylphosphatidylethanolamine (N-Suc-DSPE), N-glutaryl-distearoylphosphatidylethanolamine (N-glu-DSPE), distearoylphosphatidic acid (DSPA), and cardiolipin are also provided as anionic phospholipids.

In some embodiments, lipid nanoparticle (LNP) compositions comprising an ionizable cationic lipid compositions are provided. In some embodiments, lipid nanoparticle (LNP) compositions comprising an ionizable cationic lipid are provided. In some embodiments, the LNP composition comprises a mRNA nucleic acid. In some embodiments, a lipid nanoparticle (LNP) composition further comprises the PS lipid in a total amount of 2.5-10 mol % of the total lipid in the composition of the LNP. In some embodiments, a lipid nanoparticle (LNP) composition further comprises a PS lipid selected from the group consisting of: DSPS (L-isomer) and DPPS. In some embodiments, a lipid nanoparticle (LNP) composition comprises a conjugated lipid in a total amount of 0.5-2.0 mol % of the total lipid content of the LNP composition. In some embodiments, a lipid nanoparticle (LNP) composition comprises the conjugated lipid in a total amount of less than 2 mol % of the total lipid content of the LNP composition, and the conjugated lipid is PEG-DMG.

In some embodiments, a lipid nanoparticle (LNP) composition comprises a nucleic acid; an ionizable lipid disclosed herein; a sterol; one or more phospholipids comprising a phosphatidylserine (PS) lipid; and optionally further comprising a conjugated lipid. In some embodiments, a lipid nanoparticle (LNP) composition comprises a mRNA nucleic acid; an ionizable lipid disclosed herein; cholesterol; one or more phospholipids selected from the group consisting of: SM, DSPC, HSPC, DPPC and DOPC; and a PS lipid selected from the group consisting of: DPPS and DSPS; and optionally further comprising a conjugated lipid comprising PEG. In some embodiments, a nucleic acid lipid nanoparticle (LNP) composition comprises: a nucleic acid; an ionizable cationic lipid in a total amount of 40-65 mol % of the total lipid content of the LNP composition; a sterol in a total amount of 25-45 mol % of the total lipid content of the LNP composition; and one or more phospholipids in a total amount of phospholipids of 5-25 mol % of the total lipid content of the LNP composition, and comprising a phosphatidylserine (PS) in a total amount of 2.5-10 mol % of the total lipid content of the LNP composition; and optionally further comprising a conjugated lipid in a total amount of 0.5-2.5 mol % of the total lipid content of the LNP composition.

In some embodiments, the LNP composition further comprises an anionic lipid selected from the group consisting of: DSPS (L-isomer), DPPS (L-isomer), DMPS (L-isomer), DOPS (L-isomer), and DSPS (D-isomer).

Aspects of the disclosure relate to a lipid nanoparticle (LNP) composition comprising an ionizable lipid having the chemical structure:

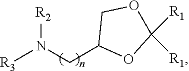

wherein
R₁ is

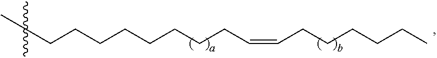

wherein a is 0 or 1; b is 1, 2, 3 or 4, provided the sum a+b is 1, 2, 3 or 4;
R₂ and R₃ are each independently (C₁-C₄)alkyl optionally substituted with hydroxyl; and
n is an integer equal to 2, 3 or 4.

In some embodiments, n is 2 or 3. In some embodiments, a is 0. In some embodiments, b is 1, 2 or 3. In some embodiments, a is 1. In some embodiments, b is 1, 2 or 3. In some embodiments, R₂ and R₃ are each methyl.

In some embodiments, R₁ is

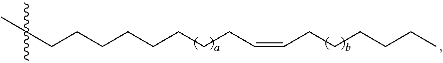

a is 0 or 1 and b is 1 or 3; R₂ and R₃ are each methyl; and n is 2 or 3. In some embodiments, n is 3.

In some embodiments, the comprises a nucleic acid; the ionizable lipid described herein, a sterol; one or more phospholipids comprising a phosphatidylserine (PS) lipid; and optionally a conjugated lipid.

In some embodiments, the nucleic acid is mRNA.

In some embodiments, the sterol is cholesterol.

In some embodiments, the one or more phospholipids consist of: one or more phospholipids selected from the group consisting of: SM, DSPC, HSPC, DPPC and DOPC; and a PS lipid selected from the group consisting of: DPPS, and DSPS.

In some embodiments, the one or more phospholipids consist of: DSPC; and one or more PS lipids selected from the group consisting of (L-Serine) DPPS and (L-Serine) DSPS.

In some embodiments, the composition comprises the PS lipid in a total amount of 2.5-10 mol % of the total lipid in the composition.

In some embodiments, the conjugated lipid comprises PEG.

Aspects of the disclosure relate to a nucleic acid lipid nanoparticle (LNP) composition comprising: a nucleic acid; an ionizable cationic lipid in a total amount of 40-65 mol % of the total lipid content of the LNP composition; a sterol in a total amount of 25-45 mol % of the total lipid content of the LNP composition; and one or more phospholipids in a total amount of phospholipids of 5-25 mol % of the total lipid content of the LNP composition, and comprising a phosphatidylserine (PS) in a total amount of 2.5-10 mol % of the total lipid content of the LNP composition; and optionally a conjugated lipid in a total amount of 0.5-2.5 mol % of the total lipid content of the LNP composition.

In some embodiments, the nucleic acid is mRNA.

In some embodiments, the sterol is cholesterol.

In some embodiments, the one or more phospholipids consist of: DSPC and a L-serine PS.

In some embodiments, the composition comprises the PS in a total amount of 2.5-7.5 mol % of the total lipid in the composition.

In some embodiments, the conjugated lipid comprises PEG. In some embodiments, conjugated lipid is PEG-DMG.

In some embodiments, the LNP comprises the conjugated lipid in a total amount of 0.5-2.0 mol % of the total lipid content of the LNP composition. In some embodiments, the conjugated lipid in a total amount of less than 2 mol % of the total lipid content of the LNP composition.

In some embodiments, the nucleic acid is a mRNA, the ionizable cationic lipid in a total amount of 45-55 mol % of the total lipid content of the LNP composition; a sterol is cholesterol in a total amount of 35-45 mol % of the total lipid content of the LNP composition; the total amount of phospholipid of 7-15 mol % of the total lipid content of the LNP composition; the one or more phospholipids consist of DSPC and the PS lipid is one or more lipids selected from the group consisting of the L-serine configuration of DPPS and DSPS; and the total amount of the PS lipid is about 5 mol % of the total lipid content of the LNP composition.

In some embodiments, the composition comprises the PS lipid in a total amount selected from 1.25 mol %, 2.5 mol %, 5 mol %, 7.5 mol %, and 10 mol % of the total lipid content of the LNP composition.

Aspects of the disclosure relate to a nucleic acid lipid nanoparticle (LNP) composition comprising: a nucleic acid, wherein the nucleic acid is mRNA; an ionizable cationic lipid, the ionizable cationic lipid in a total amount of 45-55 mol % of the total lipid content of the LNP composition; a sterol, wherein the sterol is cholesterol in a total amount of 35-45 mol % of the total lipid content of the LNP composition; one or more phospholipids, wherein the one or more phospholipids in a total amount of phospholipids of 10 mol % of the total lipid content of the LNP composition, and comprising a phosphatidylserine (PS) in a total amount of 3-9 mol % of the total lipid content of the LNP composition; and a conjugated lipid, the conjugated lipid in a total amount of 0.5-2.0 mol % of the total lipid content of the LNP composition.

In some embodiments, the one or more phospholipid is selected from the group consisting of: DSPS (L-isomer), DPPS (L-isomer), DMPS (L-isomer), DOPS (L-isomer), and DSPS (D-isomer). In some embodiments, the conjugated lipid is PEG-DMG; and the PS lipid is selected from the group consisting of: DSPS (L-isomer) and DPPS.

In some embodiments, the ionizable cationic lipid is one or more compounds selected from the group consisting of: KC3-OA, KC3-PA, KC3-C17 (8:1), and KC3-C15 (C8:1). In some embodiments, the ionizable cationic lipid is KC3-PA. In some embodiments, the ionizable cationic lipid is KC3-OA. In some embodiments, the ionizable cationic lipid is KC3-C17 (C8:1).

In some embodiments, the LNP comprises a nucleic acid; an ionizable cationic lipid in a total amount of 50 mol % of the total lipid content of the LNP composition; cholesterol in a total amount of 38.5 mol % of the total lipid content of the LNP composition; one or more phospholipids in a total amount of 7-15 mol % of the total lipid content of the LNP composition, and comprising a phosphatidylserine (PS) lipid in a total amount of 3-9 mol % of the total lipid content of the LNP composition; and a PEG-containing lipid in a total amount of 0.5-2.0 mol % of the total lipid content of the LNP composition.

In some embodiments, the phospholipids consist of one or more phospholipids selected from the group consisting of: DSPC, DOPC, DPPC, HSPC, and SM.

In some embodiments, the PS lipid is one or more L-serine lipids selected from the group consisting of DPPS and DSPS.

In some embodiments, the one or more phospholipids comprise at least two (L-Serine) PS lipids having mismatched acyl chain lengths.

In some embodiments, the phospholipids are DSPC and DPPS. In some embodiments, the DSPC and DPPS are each present in the LNP at a total amount of 5 mol % each, based on the total lipid content of the LNP composition.

Aspects of the disclosure relate to a nucleic acid lipid nanoparticle (LNP) composition comprising: a nucleic acid, ionizable cationic lipid KC3-PA or KC3-OA, and a (L-Serine) PS lipid in a total amount of 2.5-10 mol % of the total lipid content of the LNP composition.

In some embodiments, the nucleic acid is mRNA, the PS lipid is (L-Serine) DSPS, (L-Serine) DPPS, or a mixture thereof, and the LNP composition further comprises cholesterol and a second phospholipid selected from the group consisting of: DSPC, DPPC, HSPC, and SM.

In some embodiments, the LNP composition further comprises 0.5-2.0 mol % PEG-DMG or PEG-DSG, based on the total lipid content in the LNP composition.

In some embodiments, the ionizable cationic lipid is KC3-PA. In some embodiments, the ionizable cationic lipid KC3-OA.

Aspects of the disclosure relate to a nucleic acid lipid nanoparticle (LNP) composition comprising: a nucleic acid, a KC3-C17 (C8:1) ionizable cationic lipid; and a (L-Serine) PS lipid in a total amount of 2.5-10 mol % of the total lipid content of the LNP composition.

In some embodiments, the LNP composition has a N/P ratio 4 to 7. In some embodiments, the composition has a N/P ratio of 5 to 6. In some embodiments, the composition has a N/P ratio of 5.3.

Aspects of the disclosure relate to a nucleic acid lipid nanoparticle (LNP) composition comprising: a nucleic acid, ionizable cationic lipid KC3-PA, and a (L-Serine) PS lipid in a total amount of 2.5-10 mol % of the total lipid content of the LNP composition.

In some embodiments, the nucleic acid is mRNA, the PS lipid is (L-Serine) DSPS, (L-Serine) DPPS, or a mixture thereof, and the LNP composition further comprises cholesterol and a second phospholipid selected from the group consisting of: DSPC, DOPC, DPPC, HSPC, and SM.

In some embodiments, the LNP composition further comprises 0.5-2.0 mol % PEG-DMG or PEG-DSG, based on the total lipid content in the LNP composition.

Aspects of the disclosure relate to a nucleic acid lipid nanoparticle (LNP) composition comprising: a nucleic acid, an ionizable cationic lipid selected from KC3-C17 (C8:1); and a (L-Serine) PS lipid in a total amount of 2.5-10 mol % of the total lipid content of the LNP composition. In some embodiments, the N/P ratio is 4 to 7. In some embodiments, the N/P ratio is 5 to 6. In some embodiments, the N/P ratio is 3. In some embodiments, the N/P ratio is 7.

In some embodiments, the nucleic acid is mRNA encoding SARS-COV-2 spike protein.

Aspects of the disclosure relate to a nucleic acid lipid nanoparticle (LNP) vaccine composition comprising: a mRNA nucleic acid with a N/P ratio of 4 to 7; an KC3-PA ionizable cationic lipid in a total amount of 40-65 mol % of the total lipid content of the LNP composition; cholesterol in a total amount of 25-40 mol % of the total lipid content of the LNP composition; a (L-Serine) PS lipid in a total amount of 2.5-10 mol % of the total lipid content of the LNP composition; DSPC phospholipid in a total amount of 5-25 mol % of the total lipid content of the LNP composition; and PEG-DMG in a total amount of 0-2.5 mol % of the total lipid content of the LNP composition.

Aspects of the disclosure relate to a nucleic acid lipid nanoparticle (LNP) vaccine composition comprising: a mRNA nucleic acid with a N/P ratio of 3 to 8; a KC3-C17 (C8:1) ionizable cationic lipid in a total amount of 40-65 mol % of the total lipid content of the LNP composition; cholesterol in a total amount of 25-40 mol % of the total lipid content of the LNP composition; a (L-Serine) PS lipid in a total amount of 2.5-10 mol % of the total lipid content of the LNP composition; DSPC phospholipid in a total amount of 5-25 mol % of the total lipid content of the LNP composition; and PEG-DMG in a total amount of 0-2.5 mol % of the total lipid content of the LNP composition.

Aspects of the disclosure relate to a nucleic acid lipid nanoparticle (LNP) vaccine composition comprising: a mRNA nucleic acid with a N/P ratio of 4 to 7; a KC3-C15 (C8:1) ionizable cationic lipid in a total amount of 40-65 mol % of the total lipid content of the LNP composition; cholesterol in a total amount of 25-40 mol % of the total lipid content of the LNP composition; a (L-Serine) PS lipid in a total amount of 2.5-10 mol % of the total lipid content of the LNP composition; DSPC phospholipid in a total amount of 5-25 mol % of the total lipid content of the LNP composition; and PEG-DMG in a total amount of 0-2.5 mol % of the total lipid content of the LNP composition.

Aspects of the disclosure relate to a nucleic acid lipid nanoparticle (LNP) vaccine composition comprising: a mRNA nucleic acid with a N/P ratio of 3 to 8; a KC3-C18 ionizable cationic lipid in a total amount of 40-65 mol % of the total lipid content of the LNP composition; cholesterol in a total amount of 25-40 mol % of the total lipid content of the LNP composition; a (L-Serine) PS lipid in a total amount of 2.5-10 mol % of the total lipid content of the LNP composition; DSPC phospholipid in a total amount of 5-25 mol % of the total lipid content of the LNP composition; and PEG-DMG in a total amount of 0-2.5 mol % of the total lipid content of the LNP composition.

In some embodiments, the nucleic acid is a mRNA encoding a concatenated sequence of T-cell epitopes. In some embodiments, the mRNA encodes a concatenated sequence of MHC-II epitopes. In some embodiments, the mRNA encodes a concatenated sequence of MHC-I epitopes. Aspects of the disclosure relate to the use of a (L-Serine) PS lipid in combination with an ionizable cationic lipid described herein in the LNP for targeting of the LNP to dendritic cells. In some embodiments, the LNP comprises mRNA. In some embodiments, the LNP further comprises cholesterol. In some embodiments, the total amount of (L-Serine) PS lipid in the LNP is 2.5-10 mol % of the total lipid content of the LNP composition. In some embodiments, the LNP further comprises one or more additional phospholipids including DSPC. In some embodiments, the LNP further comprises a conjugated lipid. In some embodiments, the LNP comprises: a mRNA nucleic acid with a N/P ratio of 3 to 8; a KC3-PA or KC3-C17 (C8:1) ionizable cationic lipid (ICL), in a total amount of 40-65 mol % of the total lipid content of the LNP composition; cholesterol in a total amount of 25-40 mol % of the total lipid content of the LNP composition; a (L-Serine) PS lipid in a total amount of 2.5-10 mol % of the total lipid content of the LNP composition; DSPC phospholipid in a total amount of 5-25 mol % of the total lipid content of the LNP composition; and a conjugated lipid in a total amount of 0-2.5 mol % of the total lipid content of the LNP composition. In some embodiments, the ICL is KC3-PA. In some embodiments, the ICL is KC3-C17 (C8:1).

Some aspects of the disclosure relate to a lipid nanoparticle (LNP) composition comprising an ionizable lipid having the chemical structure:

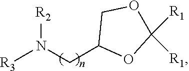

wherein
R₁ is

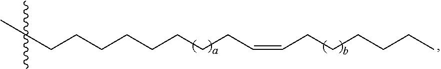

wherein a is 0 or 1; b is 1, 2, 3 or 4, provided the sum a+b is 1, 2, 3 or 4;
R₂ and R₃ are each independently methyl; and
n is an integer equal to 2 or 3.

In some embodiments, a is 0. In some embodiments, b is 1. In some embodiments, b is 3.

In some embodiments, a is 1. In some embodiments, b is 1. In some embodiments, b is 3.

In some embodiments, n is 2. In some embodiments, n is 3.

In some embodiments, the composition comprises an anionic lipid selected from the group consisting of: phosphatidylglycerol (PG), phosphatidic acid (PA), N-glutaryl-phosphatidylethanolamine (N-Glu-PE), N-succinyl-phosphatidylethanolamine (N-Suc-PE), and cardiolipin. Distearoylphosphatidylglycerol (DSPG), dipalmitoyphosphatidylglycerol (DPPG), N-succinyl-distearoylphosphatidylethanolamine (N-Suc-DSPE), N-glutaryl-distearoylphosphatidylethanolamine (N-glu-DSPE), distearoylphosphatidic acid (DSPA), and cardiolipin.

In some embodiments, the composition comprises an anionic targeting phospholipid other than phosphatidyl-L-serine.

In some embodiments, the composition comprises an anionic phospholipid selected from the group consisting of: DSPG and DPPG.

In some embodiments, the composition comprises an anionic phospholipid selected from the group consisting of: N-Glu-DSPE and N-Suc-DSPE.

In some embodiments, the composition comprises a DSPA anionic phospholipid.

In some embodiments, the composition comprises a Cardiolipin anionic phospholipid.

In some embodiments, the ionizable lipid has the chemical structure:

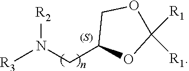

In some embodiments, the ionizable lipid has the chemical structure:

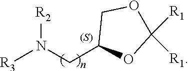

Aspects of the disclosure relates to a sodium or ammonia salt of a composition of an anionic phospholipid of Formula (V-A-1), having the chemical structure:

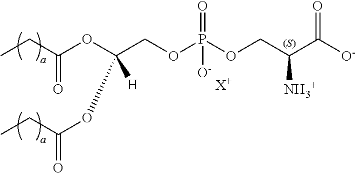

Formula (V-A-1),
wherein X′ is an ammonium cation or a sodium (Na) cation; and a is 14, 15 or 16.

In some embodiments, a is 14 or 16. In some embodiments, X⁺ is ammonium cation (NH₄⁺).

In some embodiments, X⁺ is sodium cation (Na⁺)

In some embodiments, X is an ammonium cation selected from the group consisting of: ammonium (NH₄⁺), an alkylammonium, a dialkylammonium, and a trialkylammonium salt. In some embodiments, X is X is an ammonium cation selected from the group consisting of: ammonium, dimethylamine, diethylamine, triethylamine, trimethylamine, 2-(dimethyamino)ethanol, diethanolamine, 2-(diethyamino)ethanol, ethanolamine, ethylenediamine, N-methyl-glucamine, imidazole, histidine, lysine, arginine, 4-(2-hydroxyethyl)-morpholine, piperazine, 1-(2-hydroxyethyl)-pyrrolidine, triethanolamine, and tromethamine (tris(hydroxymethyl)aminomethane).

In some embodiments, the anionic phospholipid of Formula (V-A-1) is a sodium salt of distearoylphosphatidyl-L-serine (DSPS L-isomer). In some embodiments, the anionic phospholipid of Formula (V-A-1) is an ammonium salt of distearoylphosphatidyl-L-serine (DSPS L-isomer). In some embodiments, the anionic phospholipid of Formula (V-A-1) is a sodium salt of DPPS (L-isomer). In some embodiments, the anionic phospholipid of Formula (V-A-1) is an ammonium salt of DPPS (L-isomer). Some embodiments relate to the use of the salt form composition of any one of claims 90-97 in the preparation of a liposomal nanoparticle (LNP) composition. In some embodiments, the use is in combination with one or more of the following LNP components during the preparation of the LNP composition: a mRNA nucleic acid; an ionizable cationic lipid (ICL); cholesterol; a (L-Serine) PS lipid; one or more phospholipids; and a conjugated lipid. In some embodiments, the use comprises the step of combining the ammonium or salt form of a compound of Formula (V-A-1) with one or more of the following LNP components during the preparation of the LNP composition: a mRNA nucleic acid; an ionizable cationic lipid (ICL) of any one of claim 1-9 or 73-88; cholesterol; a (L-Serine) PS lipid; one or more phospholipids; and a conjugated lipid.

In some embodiments, the LNP is a nucleic acid lipid nanoparticle vaccine composition comprising: a mRNA nucleic acid with a N/P ratio of 4 to 7; an ionizable cationic lipid of any one of claim 1-9 or 73-88 in a total amount of 40-65 mol % of the total lipid content of the LNP composition; cholesterol in a total amount of 25-40 mol % of the total lipid content of the LNP composition; a (L-Serine) PS lipid in a total amount of 2.5-10 mol % of the total lipid content of the LNP composition; DSPC phospholipid in a total amount of 5-25 mol % of the total lipid content of the LNP composition; and PEG-DMG in a total amount of 0-2.5 mol % of the total lipid content of the LNP composition.

In some embodiments, the LNP composition comprises the ionizable cationic lipid in a total amount of 46-65 mol % of the total lipid content of the LNP composition. In some embodiments, the LNP composition comprises the PS in a total amount of about 5 mol % of the total lipid in the composition. In some embodiments, the LNP composition comprises the conjugated lipid in a total amount of about 1.5 mol % of the total lipid content of the LNP composition.

In some embodiments, the conjugated lipid is PEG-DMG; and the PS lipid is selected from the group consisting of: DSPS (L-isomer) and DPPS.

In some embodiments, the ionizable cationic lipid is one or more compounds selected from the group consisting of: KC3-OA, KC3-PA, KC3-C17 (C8:1), and KC3-C15 (C8:1). In some embodiments, the ionizable cationic lipid is KC3-PA. In some embodiments, the ionizable cationic lipid is KC3-OA. In some embodiments, the ionizable cationic lipid is KC3-C17 (C8:1).

Some embodiments relate to the use of a (L-Serine) PS lipid in combination with an ionizable cationic lipid described herein in the LNP for targeting of the LNP to dendritic cells.

In some embodiments, the LNP comprises mRNA. In some embodiments, the LNP further comprises cholesterol. In some embodiments, the total amount of (L-Serine) PS lipid in the LNP is 2.5-10 mol % of the total lipid content of the LNP composition. In some embodiments, the LNP further comprises one or more additional phospholipids including DSPC. In some embodiments, the LNP further comprises a conjugated lipid.

In some embodiments, the LNP comprises: a mRNA nucleic acid with a N/P ratio of 3 to 8; a KC3-PA or KC3-C17 (C8:1) ionizable cationic lipid (ICL), in a total amount of 40-65 mol % of the total lipid content of the LNP composition; cholesterol in a total amount of 25-40 mol % of the total lipid content of the LNP composition; a (L-Serine) PS lipid in a total amount of 2.5-10 mol % of the total lipid content of the LNP composition; DSPC phospholipid in a total amount of 5-25 mol % of the total lipid content of the LNP composition; and a conjugated lipid in a total amount of 0-2.5 mol % of the total lipid content of the LNP composition.

In some embodiments, the ICL is KC3-PA. In some embodiments, the ICL is KC3-C17 (C8:1).

In some embodiments, the composition comprises an anionic phospholipid selected from the group consisting of: DSPG and DPPG, in a total amount of 2.5-7.5% of the total lipid content of the LNP composition. In some embodiments, the composition comprises DSPG anionic phospholipid in a total amount of 2.5-7.5% of the total lipid content of the LNP composition. In some embodiments, the composition comprises DPPG anionic phospholipid in a total amount of 2.5-7.5% of the total lipid content of the LNP composition.

In some embodiments, the LNP further comprises one or more additional phospholipids including DSPC.

Aspects of the disclosure relate to a nucleic acid lipid nanoparticle (LNP) composition comprising: a nucleic acid; a KC3 ionizable cationic lipid in a total amount of 40-65 mol % of the total lipid content of the LNP composition; cholesterol in a total amount of 23.5-43.5 mol % of the total lipid content of the LNP composition; a (L-Serine) PS lipid in a total amount of 2.5-10 mol % of the total lipid content of the LNP composition; DSPC or HSPC phospholipid in a total amount of 5-25 mol % of the total lipid content of the LNP composition; and a PEG-containing conjugated lipid in a total amount of 0.5 mol % to 2.5 mol % of the total lipid content of the LNP composition.

In some embodiments, the nucleic acid is mRNA. In some embodiments, the N/P ratio is 3 to 8.

In some embodiments, the KC3 ionizable cationic lipid is selected from the group consisting of: KC3-OA, KC3-PA, KC3-C17 (8:1), and KC3-C15 (C8:1). In some embodiments, the KC3 ionizable cationic lipid is KC3-OA. In some embodiments, the KC3 ionizable cationic lipid is KC3-PA. In some embodiments, the KC3 ionizable cationic lipid is KC3-C17 (C8:1). In some embodiments, the KC3 ionizable cationic lipid is KC3-C15 (C8:1).

In some embodiments, the conjugated lipid is PEG-DMG or PEG-DSG.

In some embodiments, the composition comprises the PEG-containing conjugated lipid in a total amount of 0.5-2.0 mol % of the total lipid content of the LNP composition.

In some embodiments, the composition comprises the KC3 ionizable cationic lipid in a total amount of 48 mol % of the total lipid content of the LNP composition.

In some embodiments, the composition comprises DSPC and DSPS in a total amount of 10 mol % of the total lipid content of the LNP composition.

In some embodiments, the composition comprises 5% DSPC or HSPC in a total amount of 5 mol % of the total lipid content of the LNP composition.

In some embodiments, the composition comprises PEG-DMG in a total of 1.5 mol % of the total lipid content of the LNP composition.

In some embodiments, the composition comprises cholesterol in a total amount of 40.5 mol % cholesterol of the total lipid content of the LNP composition.

In some embodiments, the composition comprises the DSPC phospholipid in a total amount of 10 mol % of the total lipid content of the LNP composition.

In some embodiments, the PEG-containing conjugated lipid is PEG₂₀₀₀-DMG.

In some embodiments, the composition comprises the cholesterol in a total amount of 23.5 mol % of the total lipid content of the LNP composition. In some embodiments, the composition comprises the cholesterol in a total amount of 33.5 mol % of the total lipid content of the LNP composition. In some embodiments, the composition comprises the cholesterol in a total amount of 38.5 mol % of the total lipid content of the LNP composition. In some embodiments, the composition comprises the cholesterol in a total amount of 40.5 mol % of the total lipid content of the LNP composition. In some embodiments, the composition comprises the cholesterol in a total amount of 42.7 mol % of the total lipid content of the LNP composition. In some embodiments, the composition comprises the cholesterol in a total amount of 43.5 mol % of the total lipid content of the LNP composition. In some embodiments, the composition comprises the cholesterol in a total amount of 33.5-43.5 mol % of the total lipid content of the LNP composition. In some embodiments, the composition comprises the KC3 ionizable cationic lipid in a total amount of 45-55 mol % of the total lipid content of the LNP composition.

Aspects of the disclosure relate to a nucleic acid lipid nanoparticle (LNP) composition comprising: a mRNA nucleic acid; a KC3 ionizable cationic lipid selected from the group consisting of: KC3-OA, KC3-PA, KC3-C17 (8:1), and KC3-C15 (C8:1), in a total amount of 45-55 mol % of the total lipid content of the LNP composition; cholesterol in a total amount of 33.5-43.5 mol % of the total lipid content of the LNP composition; a (L-Serine) DPPS lipid in a total amount of 5 mol % of the total lipid content of the LNP composition; DSPC or HSPC phospholipid in a total amount of 5 mol % of the total lipid content of the LNP composition; and a PEG-DMG conjugated lipid in a total amount of 1.5 mol % of the total lipid content of the LNP composition.

Aspects of the disclosure relate to a lipid nanoparticle (LNP) composition comprising a KC3 ionizable cationic lipid, a (L-Serine) PS lipid, cholesterol, one or more phospholipids comprising at least one anionic phospholipid, and a conjugated lipid, wherein the LNP is obtained by a process comprising the step of dissolving a sodium or ammonium salt of the anionic phospholipid.

In some embodiments, the composition comprises a nucleic acid. In some embodiments, the nucleic acid is mRNA.

In some embodiments, the composition is a vaccine. In some embodiments, the composition is an injectable vaccine composition.

In some embodiments, the total amount of phospholipids in the composition is 5-25 mol % of the total lipid content of the LNP composition, and the total amount of the phosphatidylserine (PS) is 2.5-10 mol % of the total lipid content of the LNP composition; and the total amount of the conjugated lipid in the composition is a total amount of 0.5-2.5 mol % of the total lipid content of the LNP composition.

In some embodiments, the composition comprises 48 mol % of the KC3 ionizable cationic lipid, 40.5 mol % cholesterol, and 5 mol % (L-Serine) DPPS lipid, wherein each mol % refers to the mol % of the total lipid content of the LNP composition.

In some embodiments, the composition comprises 48 mol % of the KC3 ionizable cationic lipid, 38.5 mol % cholesterol, and 5 mol % (L-Serine) DPPS lipid, wherein each mol % refers to the mol % of the total lipid content of the LNP composition.

In some embodiments, the composition comprises 46-54 mol % of the KC3 ionizable cationic lipid, and 5 mol % (L-Serine) DPPS lipid, wherein each mol % refers to the mol % of the total lipid content of the LNP composition.

In some embodiments, the composition comprises 45 mol % of the KC3 ionizable cationic lipid, 42.7 mol % cholesterol, and 5 mol % (L-Serine) DPPS lipid, wherein each mol % refers to the mol % of the total lipid content of the LNP composition.

In some embodiments, the composition comprises 50 mol % of the KC3 ionizable cationic lipid, 38.5 mol % cholesterol, 5 mol % (L-Serine) DPPS lipid, and a total of 10 mol % phospholipid concentration; wherein each mol % refers to the mol % of the total lipid content of the LNP composition.

In some embodiments, the composition comprises 48 mol % of the KC3 ionizable cationic lipid, 40.5 mol % cholesterol, 5 mol % (L-Serine) DPPS lipid, and a total of 10 mol % phospholipid concentration; wherein each mol % refers to the mol % of the total lipid content of the LNP composition.

In some embodiments, the composition comprises 48 mol % of the KC3 ionizable cationic lipid, 40.5 mol % cholesterol, 5 mol % (L-Serine) DPPS lipid, 5 mol % DSPC or DPPC; and a total of 10 mol % phospholipid concentration; wherein each mol % refers to the mol % of the total lipid content of the LNP composition.

In some embodiments, the composition comprises 46.5 mol % of the KC3 ionizable cationic lipid, 42 mol % cholesterol, 5 mol % (L-Serine) DPPS lipid, wherein each mol % refers to the mol % of the total lipid content of the LNP composition.

In some embodiments, the composition further comprises a total of 5 mol % DSPC or HSPC of the total lipid content of the LNP composition.

In some embodiments, the composition further comprises a total of 1.5 mol % PEG-DMG of the total lipid content of the LNP composition.

In some embodiments, the composition comprises a total of 10 mol % of DSPC/DPPC phospholipid of the total lipid content of the LNP composition.

Aspects of the disclosure relate to a phosphatidylserine salt selected from the group consisting of DSPS sodium, DPPS sodium, DSPS ammonium and DPPS ammonium.

Aspects of the disclosure relate to the use of a DSPS-Na salt or a DPPS-NH₄ salt in the preparation of a LNP comprising a (L-Serine) PS lipid, a sterol, a conjugated lipid, a phospholipid for targeting the LNP to dendritic cells.

Aspects of the disclosure relate to a solution comprising ethanol and DSPS or DPPS, the solution obtained by a process comprising the step of dissolving a phosphatidylserine salt in ethanol, wherein the phosphatidylserine salt is selected from the group consisting of DSPS sodium, DPPS sodium, DSPS ammonium and DPPS ammonium.

### BRIEF DESCRIPTION OF THE DRAWINGS

FIG. 1A Impact of DSPS inclusion from 0-2.5 mol % on transfection efficiency of dendritic cells (MutuDC1940) using mCherry mRNA LNPs formulated with DLin-KC2-DMA as the ionizable cationic lipid. ICL was kept at 50 mol %, cholesterol at 38.5 mol %, PEG-DMG at 1.5 mol % and the DSPS content varied. Inclusion of DSPS was made by reducing the DSPC content by the same mol % of DSPS that was added. Cells were incubated with each formulation at a concentration of 1 ug mRNA/mL for 24 h. UT sample corresponds to cells where no LNPs were added. Lipofect refers to Lipofectamine treated sample.

FIG. 1B Impact of DSPS inclusion from 0-7.5 mol % on transfection efficiency of dendritic cells (MutuDC1940) using mCherry mRNA LNPs formulated with DLin-KC2-DMA as the ionizable cationic lipid. ICL was kept at 50 mol %, cholesterol at 38.5 mol %, PEG-DMG at 1.5 mol % and the DSPS content varied. Inclusion of DSPS was made by reducing the DSPC content by the same mol % of DSPS that was added. Cells were incubated with each formulation at a concentration of 1 μg mRNA/mL for 24 h. UT sample corresponds to cells where no LNPs were added. Lipofect refers to Lipofectamine treated sample.

FIG. 1C Impact of DSPS inclusion from 0-7.5 mol % on transfection efficiency of dendritic cells (MutuDC1940) using mCherry mRNA LNPs formulated with DLin-KC2-DMA as the ionizable cationic lipid. ICL was kept at 50 mol %, cholesterol at 38.5 mol %, PEG-DMG at 1.5 mol % and the DSPS content varied. Inclusion of DSPS was made by reducing the DSPC content by the same mol % of DSPS that was added. Cells were incubated with each formulation at a concentration of 0.3 ug mRNA/mL for 24 h. UT sample corresponds to where no LNPs were added.

FIG. 1D Impact of DSPS inclusion from 0-7.5 mol % on transfection efficiency of dendritic cells (MutuDC1940) using mCherry mRNA LNPs formulated with DLin-KC2-DMA as the ionizable cationic lipid. ICL was kept at 50 mol %, cholesterol at 38.5 mol %, PEG-DMG at 1.5 mol % and the DSPS content varied. Inclusion of DSPS was made by reducing the DSPC content by the same mol % of DSPS that was added Cells were incubated with each formulation at a concentration of 0.1 ug mRNA/mL for 24 h. UT sample corresponds to cells where no LNPs were added.

FIG. 2 Transfection of murine dendritic cells (MutuDC1940) using LNPs containing various ICLs (KC2, KC2-OA, KC3-OA, and SM-102) and 5 mol % DSPS, and comparison to LNPs using Glu-DSPE or Suc-DSPE rather than DSPS. UT sample corresponds to cells where no LNPs were added.

FIG. 3 DSPS or DPPS increase mCherry LNP transfection with KC2, KC2-01, KC2-PA, KC3-01, and KC3-OA comprising ICLs. UT sample corresponds to cells where no LNPs were added.

FIG. 4 Impact of PEG-DMG concentration in AUG-UO-1 containing LNPs with and without 5 mol % DSPS on transfection of dendritic cells. The Y-axis shows the % PEG used in the composition followed by the concentration of mRNA added to the cells (0.11, 0.33, or 1 μg/mL). UT sample corresponds to cells where no LNPs were added.

FIG. 5A Effect of N/P on mCherry expression of KC2-01 containing LNPs at 1 μg/ml in murine dendritic cells. UT sample corresponds to cells where no LNPs were added.

FIG. 5B Effect of N/P on mCherry expression of KC2-01 containing LNPs at 0.33 μg/ml in murine dendritic cells. UT sample corresponds to cells where no LNPs were added.

FIG. 6 Transfection efficiency of LNP formulations containing various concentrations of DOPS (0, 10, and 25 mol % as % of total lipid) and mCherry mRNA in murine dendritic cells.

FIG. 7A mRNA sequence of VRN-029, a SARS-COV2 spike protein generating sequence.

FIG. 7B The effect of PEG-DMG (C14) concentration (mol %) on LNP vaccine immunogenicity. Total anti-spike antibody titers and CD4 responses from mice immunized with mRNA-LNPs using 7.5% DSPS and the ionizable lipid UO1 with increasing mol % of PEG-DMG. The middle graph shows day 34 endpoint antibody titers. The right graph shows the corresponding CD4 T cell responses.

FIG. 7C The effect of PEG-DPPE (C16) concentration (mol %) on LNP vaccine immunogenicity. Total anti-spike antibody titers from mice immunized with mRNA-LNPs using 7.5% DSPS and the ionizable lipid UO1 with increasing mol % of PEG-DPPE. The middle graph shows day 34 endpoint antibody titers. The mol % of PEG-DPPE inversely impacted antibody levels. The right graph shows the corresponding CD4 T cell responses.

FIG. 7D Total anti-spike antibody titers and CD4 responses from mice immunized with mRNA-LNPs using 7.5% DSPS and the ionizable lipid KC20A with either 1.5 mol % PEG-DMG (14C) or PEG-DSG (18C). The left graph shows day 34 endpoint antibody titers. The right graph shows the corresponding CD4 T cell responses.

FIG. 7E Total anti-spike antibody titers and CD4 responses from mice immunized with mRNA-LNPs using 7.5% DSPS and the ionizable lipid UO1 with either 1.5 mol % PEG-DMG (14C) or PEG-DSG (18C). The left graph shows day 34 endpoint antibody titers. The right graph shows the corresponding CD4 T cell responses.

FIG. 7F Effect of phosphatidylserine incorporation in mRNA-LNP immunogenicity. Total anti-spike antibody titers (A) and spike-specific CD4 T cell responses from mice immunized with mRNA-LNPs using various ionizable lipids and PEG-lipids plus/minus 7.5 mol % DSPS Antibody data were log-transformed and analyzed using two-way ANOVA with a Sidak's multiple comparison test. CD4 T cell data were analyzed using a REML mixed-effects model with a Sidak's multiple comparison test.

FIG. 7G Effect of phosphatidylserine lipid tail (DPPS vs DSPS) composition on mRNA-LNP priming of B (Panel A) and T cell (Panel B) responses. Antibody data were log-transformed prior to analysis. Data were analyzed using one-way ANOVA with a Tukey's multiple comparison test.

FIG. 8A Comparison of the mCherry expression of KC2-01 LNPs, 7.5 mol % DSPS (D isomer) and DSPS (L isomer) at 1 μg/mL mRNA for 24 h.

FIG. 8B Comparison of the mCherry expression of KC2-01 LNPs, 7.5 mol % DSPS (D isomer) and DSPS (L isomer) at 0.33 μg/mL mRNA for 24 h.

FIG. 9 Comparison of the mCherry expression of KC2 LNPs, with 5 and 7.5 mol % DSPS (L-isomer) to LNPs prepared with SM-102 or ALC-0315 at 1 μg/mL mRNA for 24 h. The Y-axis is mean fluorescence intensity (MFI). UT sample corresponds to cells where no LNPs were added.

FIG. 10 Comparison of the mCherry expression of UO1, SM102, ALC-0315 formulations alone, or with added DSPS, at 1 μg/mL mRNA for 24 h. Lipo refers to Lipofectamine MessengerMax (ThermoFisher) used according to manufacturer's instructions at the same dosage level as the LNPs. UT sample corresponds to cells where no LNPs were added.

FIG. 11 Oxidative degradation of liposomes containing KC3 (DLin-KC3-DMA), a polyunsaturated ICL with a single methylene between two olefins, to liposomes containing ICLs with monounsaturated alkyl chains (KC3-OA, KC3-PA, or KC3-C17 (C8:1)) and the fully saturated ICL, KC3-C17. Effect of hydrogen peroxide on the stability of individual ionizable cationic lipids measured by CAD-HPLC.

FIG. 12 Comparison of the mCherry expression in murine dendritic cells of LNPs containing the polyunsaturated KC3, the monounsaturated KC3-OA, KC3-PA, or KC3C17 (C8:1), and the fully saturated KC3C17, all with or without DPPS (NH₄⁺ salt), at 0.3 or 1 μg/mL mRNA for 24 h. UT sample corresponds to cells where no LNPs were added.

FIG. 13 Comparison of the mCherry expression in human dendritic cells of LNPs containing the polyunsaturated KC2 or KC3 with a single methylene between two olefins, polyunsaturated KC3-01 with four methylenes between two olefins, monounsaturated KC3-OA, KC3-PA, or KC3C17 (C8:1), and ALC-0315, all with except ALC-0315 with DPPS (NH₄⁺ salt), at 0.1 or 1 μg/mL mRNA for 24 h. Untreated samples correspond to cells where no LNPs were added.

FIG. 14 Comparison of the mCherry expression of LNP formulations with 5 mol % DSPS and 46-54 mol % of KC3-OA to ALC-0315 and SM-102 LNP controls, at 0.1 and 1 μg/mL mRNA for 24 h in human dendritic cells. Untreated DC sample corresponds to human dendritic cells where no LNPs were added.

FIG. 15 Comparison of the mCherry expression of LNP formulations with 0 or 5 mol % DSPS and 50 mol % KC2-01 at N/P ratios of 4-7, at 0.1 μg/mL mRNA for 24 h in human dendritic cells. These were also compared to LNPs containing KC3-OA and 5 mol % DSPS at N/P of 5. Untreated DC sample corresponds to human dendritic cells where no LNPs were added.

FIG. 16A Comparison of polyunsaturated KC3 with monounsaturated KC3-OA and KC3-PA containing LNP formulations on vaccine immunogenicity. Total anti-spike antibody titers from mice immunized with mRNA-LNPs using 5 mol % DSPS or DPPS-targeted LNPs containing either KC3, KC3-OA, or KC3-PA. For KC3-OA and KC3-PA LNPs, each formulation was also evaluated with either the C16 DPPC or C18 DSPC neutral phosphatidylcholine component. All LNPs contained 1.5 mol % of PEG-DMG. The graph shows day 21 endpoint antibody titers after the initial prime injection of 1 μg mRNA per mouse.

FIG. 16B Comparison of polyunsaturated KC3 with monounsaturated KC3-OA and KC3-PA containing LNP formulations on vaccine immunogenicity. Total anti-spike antibody titers from mice immunized with mRNA-LNPs using 5 mol % DSPS or DPPS-targeted LNPs containing either KC3, KC3-OA, or KC3-PA. For KC3-OA and KC3-PA LNPs, each formulation was also evaluated with either the C16 DPPC or C18 DSPC neutral phosphatidylcholine component. All LNPs contained 1.5 mol % of PEG-DMG. The graph shows day 34 endpoint antibody titers after the prime then boost on day 21 of 1 μg mRNA per mouse.

FIG. 17A Comparison of the mCherry expression of LNP formulations with 5 mol % DSPS and 43-48 mol % of KC3-OA to ALC-0315 and SM-102 LNP controls, at 1 μg/mL mRNA for 24 h in human dendritic cells KC3-OA LNPs prepared at 45 mol % KC3-OA and 5 mol % DSPS of total lipid were also compared at N/P ratios of 5, 5.5, 6.0, and 6.5. Finally, LNPs with 45 mol % KC3-OA at N/P of 5 and 6 were evaluated with PEG-SA, at either 1 or 3 mol %, in place of 1.5 mol % PEG-DMG. Untreated DC sample corresponds to human dendritic cells where no LNPs were added.

FIG. 17B Comparison of the mCherry expression of LNP formulations with 5 mol % DSPS and 43-48 mol % of KC3-OA to ALC-0315 and SM-102 LNP controls, at 0.1 μg/mL mRNA for 24 h in human dendritic cells KC3-OA LNPs prepared at 45 mol % KC3-OA and 5 mol % DSPS of total lipid were also compared at N/P ratios of 5, 5.5, 6.0, and 6.5. Finally, LNPs with 45 mol % KC3-OA at N/P of 5 and 6 were evaluated with PEG-SA, at either 1 or 3 mol %, in place of 1.5 mol % PEG-DMG. Untreated DC sample corresponds to human dendritic cells where no LNPs were added.

FIG. 18A Comparison of the mCherry expression of UO-1 or KC3-01 containing LNP formulations with 0-10 mol % of DSPG in human dendritic cells following incubation for 24 h at 1 μg/mL mRNA. ALC-0315 and SM-102 LNPs controls were also included at 1 μg/mL mRNA and untreated DC sample corresponds to human dendritic cells where no LNPs were added.

FIG. 18B Comparison of the mCherry expression of UO-1 or KC3-01 containing LNP formulations with 0-10 mol % of DSPG in human dendritic cells following incubation for 24 h at 0.1 μg/mL mRNA. ALC-0315 and SM-102 LNPs controls were also included at 0.1 μg/mL mRNA and untreated DC sample corresponds to human dendritic cells where no LNPs were added.

FIG. 19 Comparison of dilinoleyl KC2, monounsaturated KC3-OA, and four methylene interrupted poly unsaturated ICLs (KC3-01, AKG-UO1, and AKG-U09) containing LNP formulations on vaccine immunogenicity. ALC-0315 containing LNPs were included as a control. All LNPs contained 1.5 mol % of PEG-DMG. Total anti-spike antibody titers from mice immunized with mRNA-LNPs were determined on day 21 after the initial prime injection of 1 μg mRNA per mouse on day 1.

FIG. 20A Comparison of the mCherry expression of 48 mol % KC3-OA containing LNP formulations with 5 mol % of various anionic phospholipids in human dendritic cells following incubation for 24 h at 1 μg/mL mRNA. All LNPs included 2.5 mol % of DSPC, 50 mol % of UO-1, and 1.5 mol % of PEG-DMG. The anionic phospholipids included the phosphatidylglycerols, DOPG, DSPG, DPPG, and DMPG, as well as DSPS. In some LNPs, the DSPG and DSPS were combined either alone or together with DSPC. Two donors were used to produce human dendritic cells in this study and untreated DC sample corresponds to human dendritic cells where no LNPs were added.

FIG. 20B Comparison of the mCherry expression of 48 mol % KC3-OA containing LNP formulations with 5 mol % of various anionic phospholipids in human dendritic cells following incubation for 24 h at 0.1 μg/mL mRNA. All LNPs included 2.5 mol % of DSPC, 50 mol % of UO-1, and 1.5 mol % of PEG-DMG. The anionic phospholipids included the phosphatidylglycerols, DOPG, DSPG, DPPG, and DMPG, as well as DSPS. In some LNPs, the DSPG and DSPS were combined either alone or together with DSPC. Two donors were used to produce human dendritic cells in this study and untreated DC sample corresponds to human dendritic cells where no LNPs were added.

FIG. 21 Comparison of the mCherry expression in murine dendritic cells of LNPs containing KC3-OA LNPs with either 5 mol % DSPS (Na⁺ salt) or 5 mol % DPPS (NH₄⁺ salt) after incubation at 1 μg/mL mRNA for 24 h. ALC-0315 and SM-102 LNPs controls were also included at 1 μg/mL mRNA. UT sample corresponds to cells where no LNPs were added.

FIG. 22A Immunogenicity of mRNA-LNPs vaccines encoding Mtb antigens containing four unique signal peptides and comparison of KC3OA/DPPS and ALC-0315 LNP formulations. Mtb-specific CD4 T cells were defined as any cell that produced either of these 3 cytokines following peptide stimulation.

FIG. 22B Immunogenicity of mRNA-LNPs vaccines encoding Mtb antigens containing four unique signal peptides and comparison of KC3OA/DPPS and ALC-0315 LNP formulations. Mtb-specific CD8 T cells were identified as any cell that produced IFN-γ; TNF-α and IL-2 producing CD8 T cells were found within the IFN-γ-producing population.

FIG. 23A Proportion of total vaccine-induced T cell response to individual or subsets of Mtb antigens. Data correspond to cumulative T cell responses shown in FIG. 22 and are normalized to 100%. Profile of CD4 T cell responses using the ALC-0315 comparator.

FIG. 23B Proportion of total vaccine-induced T cell response to individual or subsets of Mtb antigens. Data correspond to cumulative T cell responses shown in FIG. 22 and are normalized to 100%. Profile of CD4 T cell responses using the KC3-OA/DPPS LNP formulation.

FIG. 23C Proportion of total vaccine-induced T cell response to individual or subsets of Mtb antigens. Data correspond to cumulative T cell responses shown in FIG. 22 and are normalized to 100%. Profile of CD8 T cell responses using the ALC-0315 comparator.

FIG. 23D Proportion of total vaccine-induced T cell response to individual or subsets of Mtb antigens. Data correspond to cumulative T cell responses shown in FIG. 22 and are normalized to 100%. Profile of CD8 T cell responses using the KC3-OA/DPPS LNP formulation.

FIG. 24A Cytokine polyfunctionality of vaccine-specific CD4 T cells where CD4 T cell responses were induced by the mRNA incorporating sec/MITD targeting of nascent proteins to the endosomal compartment and signal peptide/transmembrane domain into the LNP formulation. Concatenated CD4 T cell responses across peptide pools were Boolean gated on cells that produced IFN-γ, IL-2 and TNF-α. SP, single producer; DP, double producer; TP, triple producer.

FIG. 24B Cytokine polyfunctionality of vaccine-specific CD4 T cells where CD4 T cell responses were induced by the mRNA incorporating the LAMP-1 targeting of nascent proteins to the late endosomal/lysosomal compartment into the LNP formulation. Concatenated CD4 T cell responses across peptide pools were Boolean gated on cells that produced IFN-γ, IL-2 and TNF-α. SP, single producer; DP, double producer; TP, triple producer.

FIG. 24C Cytokine polyfunctionality of vaccine-specific CD4 T cells where CD4 T cell responses were induced by the mRNA using the tPA signal peptide that directs proteins to be secreted into the LNP formulation. Concatenated CD4 T cell responses across peptide pools were Boolean gated on cells that produced IFN-γ, IL-2 and TNF-α. SP, single producer; DP, double producer; TP, triple producer.

FIG. 25A Total CD4 T cell responses (cell IFN-γ) induced by mRNA delivered with the KC3-OA/DPPS or ALC-0315 LNP formulation. Data correspond to cumulative T cell responses shown in FIG. 22. Mtb-specific T cell responses were concatenated across peptide pools.

FIG. 25B Total CD8 T cell responses (cell IFN-γ) induced by mRNA delivered with the KC3-OA/DPPS or ALC-0315 LNP formulation. Data correspond to cumulative T cell responses shown in FIG. 22. Mtb-specific T cell responses were concatenated across peptide pools.

FIG. 26A Comparison of BCG (s.c.) with KC3-OA/DPPS LNP (i.m.) CD4 T cell responses to individual Mtb antigens. Stim 1: EsxH/TB10.4 and Ag85B peptide pools, Stim 2: Mtb39a peptide pool, Stim 3: EsxW and EsxV peptide pools, Stim 4: EsxB/CFP10 and EsxA/ESAT-6 peptide pools, Stim 5: C-terminal set of ten tandem 15mer minimal epitope peptide pool. Mtb-specific CD4 T cells were defined as cells expressing IFN-γ, TNF-α, IL-2, IL-17a or combinations thereof.

FIG. 26B Comparison of BCG (s.c.) with KC3-OA/DPPS LNP (i.m.) CD4 T cell responses. Cumulative CD4 T cell response from all peptide stimulations (Sim 1+Stim 2+Stim 3+Stim 4+Stim 5-background). Mtb-specific CD4 T cells were defined as cells expressing IFN-γ, TNF-α, IL-2, IL-17a or combinations thereof.

FIG. 27A Comparison of BCG (s.c.) with KC3-OA/DPPS LNP (i.m.) CD8 T cell responses to individual Mtb antigens. Stim 1: EsxH/TB10.4 and Ag85B peptide pools, Stim 2: Mtb39a peptide pool, Stim 3: EsxW and EsxV peptide pools, Stim 4: EsxB/CFP10 and EsxA/ESAT-6 peptide pools, Stim 5: C-terminal set of ten tandem 15mer minimal epitope peptide pool. Mtb-specific CD8 T cells expressing IFN-γ, TNF-α, IL-2, IL-17a or combinations thereof.

FIG. 27B Comparison of BCG (s.c.) with KC3-OA/DPPS LNP (i.m.) CD8 T cell responses. Cumulative CD8 T cell response from all peptide stimulations (Sim 1+Stim 2+Stim 3+Stim 4+Stim 5-background).

FIG. 28 Vaccination with three different mRNA constructs encoding putative human MHC class I-restricted Mtb epitopes in a string-on-bead format generates antigen-specific CD8 T cell responses in CB6F1 mice.

FIG. 29A Kinetics of vaccine-specific T cell responses. CB6F1 mice were immunized with mRNA encoding for an HLA-II directed fusion protein consisting of 7 Mtb proteins plus 10 minimal epitopes; the antigen was flanked with sec/MITD sequences (SEQ ID NOs. 37 and 38), encapsulated in KC3-OA/DPPS LNPs and boosted 4 weeks later. The cumulative CD4 and CD8 T cell responses to all peptide pools are shown.

FIG. 29B Kinetics of vaccine-specific CD8 T-cell responses following immunization with mRNA encoding putative human MHC class I Mtb epitopes encapsulated in KC3-OA/DPPS LNPs and boosted 4 weeks later. The cumulative CD8 T cell responses to all peptide pools are shown.

FIG. 30A Comparison of CD4 T cell responses between a 1st and 2nd generation HLA-II Mtb mRNA vaccine construct encapsulated in KC3-OA/DPPS LNPs showing the cumulative total of the CD4 T-cell response (sum of all individual peptide pools minus the background) following immunization of CB6F1 mice. One group was vaccinated with mRNA formulated with KC3-OA/DPPS LNPs containing an increased amount of 15 mol % DSPC (at the expense of cholesterol) versus the typical 5 mol % DSPC.

FIG. 30B Comparison of CD4 T cell responses between a 1^{st} and 2^{nd} generation HLA-II Mtb mRNA vaccine construct encapsulated in KC3-OA/DPPS LNPs showing the proportion of total vaccine-induced CD4 T cell responses to individual or subsets of Mtb antigens encoded by the mRNA following immunization of CB6F1 mice. Data correspond to cumulative T cell responses shown in (FIG. 30A) and are normalized to 100%. One group was vaccinated with mRNA formulated with KC3-OA/DPPS LNPs containing an increased amount of 15 mol % DSPC (at the expense of cholesterol) versus the typical 5 mol % DSPC.

FIG. 31A Comparison of CD8 T-cell responses between a 1^{st} and 2^{nd} generation HLA-II Mtb mRNA vaccine construct encapsulated in KC3-OA/DPPS LNPs showing the cumulative total of the CD8 T-cell response (sum of all individual peptide pools minus the background) following immunization of CB6F1 mice. One group was vaccinated with mRNA formulated with KC3-OA/DPPS LNPs containing an increased amount of 15 mol % DSPC (at the expense of cholesterol) versus the typical 5 mol % DSPC.

FIG. 31B Comparison of CD8 T-cell responses between a 1^{st} and 2^{nd} generation HLA-II Mtb mRNA vaccine construct encapsulated in KC3-OA/DPPS LNPs showing the proportion of total vaccine-induced CD8 T-cell responses to individual or subsets of Mtb antigens encoded by the mRNA following immunization of CB6F1 mice. Data correspond to cumulative T cell responses shown in (FIG. 31A) and are normalized to 100%. One group was vaccinated with mRNA formulated with KC3-OA/DPPS LNPs containing an increased amount of 15 mol % DSPC (at the expense of cholesterol) versus the typical 5 mol % DSPC.

FIG. 32 is a scheme showing the synthesis of 2-((S)-2,2-di((6Z,12Z)-octadeca-6,12-dien-1-yl)-1,3-dioxolan-4-yl)-N,N-dimethylethan-1-amine (AKG-KC2-01) and 3-((S)-2,2-di((6Z,12Z)-octadeca-6,12-dien-1-yl)-1,3-dioxolan-4-yl)-N,N-dimethylpropan-1-amine (AKG-KC3-01) according to some embodiments of the disclosure.

FIG. 33 is a scheme showing the synthesis 2-((S)-2,2-di((Z)-octadec-9-en-1-yl)-1,3-dioxolan-4-yl)-N,N-dimethylethan-1-amine (AKG-KC2-OA), 2-((S)-2,2-di((Z)-hexadec-9-en-1-yl)-1,3-dioxolan-4-yl)-N,N-dimethylethan-1-amine (AKG-KC2-PA), 3-((S)-2,2-di((Z)-octadec-9-en-1-yl)-1,3-dioxolan-4-yl)-N,N-dimethylpropan-1-amine (AKG-KC3-OA), and 3-((S)-2,2-di((Z)-hexadec-9-en-1-yl)-1,3-dioxolan-4-yl)-N,N-dimethylpropan-1-amine, (AKG-KC3-PA, O-12418) according to some embodiments of the disclosure.

FIG. 34 is a scheme showing the synthesis of 3-((S)-2,2-di((Z)-heptadec-8-en-1-yl)-1,3-dioxolan-4-yl)-N,N-dimethylpropan-1-amine, AKG-KC3-C17 (C8:1) and(S)-3-(2,2-diheptadecyl-1,3-dioxolan-4-yl)-N,N-dimethylpropan-1-amine, AKG-KC3-C17 according to some embodiments of the disclosure.

FIG. 35A Comparison of splenic CD4 T cell responses in CB6F1 mice after vaccination with a 2^{nd} generation HLA-II Mtb mRNA formulated in LNPs containing increasing amounts of phospholipid (PL). DPPS was held constant at 5 mol % and the remaining mol % of PL consisted of DSPC (e.g. 10 mol % PL consists of 5 mol % DPPS and 5 mol % DSPC). DSPC content increased at the expense of cholesterol.

FIG. 35B Comparison of splenic CD8 T cell responses in CB6F1 mice after vaccination with a 2^{nd} generation HLA-II Mtb mRNA formulated in LNPs containing increasing amounts of PL. CD8 T cell responses were quantified in the same mice as in FIG. 35A.

FIG. 35C Comparison of splenic CD4 T cell responses in CB6F1 mice after vaccination with a 2^{nd} generation HLA-II Mtb mRNA formulated in LNPs containing increasing amounts of PL. DPPS was held constant at 5 mol % and the remaining mol % of PL consisted of DSPC. One group was immunized with mRNA produced with unmodified uridine formulated in 25 mol % PL; all other groups received mRNA with all uridines replaced with N1-methylpseudouridine.

FIG. 35D Comparison of splenic CD8 T cell responses in CB6F1 mice after vaccination with a 2^{nd} generation HLA-II Mtb mRNA formulated in LNPs containing increasing amounts of PL. CD8 T cell responses were quantified in the same mice as in FIG. 35C.

FIG. 36A Comparison of splenic CD8 T cell responses in CB6F1 mice after vaccination with a 2^{nd} generation HLA-I “Mixed” mRNA formulated in LNPs containing increasing amounts of PL. DPPS was held constant at 5 mol % and the remaining mol % of PL consisted of DSPC.

FIG. 36B Comparison of splenic CD8 T cell responses in CB6F1 mice after vaccination with a 2^{nd} generation HLA-I “Mtb-only” mRNA formulated in LNPs containing increasing amounts of PL. DPPS was held constant at 5 mol % and the remaining mol % of PL consisted of DSPC.

### DETAILED DESCRIPTION

It is to be understood that both the foregoing general description and the following detailed description are exemplary and explanatory only and are not restrictive of the compositions and methods of the present disclosure.

Liposomal nanoparticle (LNP) compositions can comprise an ionizable lipid, a sterol, and one or more phospholipids. In some embodiments, the LNP compositions further comprise a nucleic acid such as mRNA for administration in a pharmaceutical composition such as a vaccine. In some embodiments, the LNP compositions optionally further comprise a conjugated lipid.

Lipid Nanoparticle (LNP) compositions comprising mRNA include Stabilized Nucleic Acid Lipid Particles (SNALP) used as a vehicle for the systemic delivery of mRNA or other nucleic acid therapeutics. SNALP compositions include cationic lipids such as MC3 or KC2, comprising a protonatable tertiary amine head group joined to a pair of linear 18 carbon aliphatic chains containing a pair of carbon-carbon double bonds separated by a single methylene group (e.g., linoleic acid). However, while the structure of these hydrocarbon chains, each containing a pair of double bonds separated by a single methylene group, imparts desirable biological properties to the SNALP compositions, this chemical sub-structure also results in the undesired problem of increased sensitivity of the compound to oxidative degradation. What is needed are novel cationic lipids suitable for use in a SNALP composition, but having enhanced resistance to oxidative degradation.

Aspects of the present disclosure relates to dendritic-cell targeted lipid nanoparticles (LNP) incorporating mRNA encoding for combinations of specific CD8 and CD4 T-cell epitopes found in Mycobacterium tuberculosis. In some embodiments, a LNP comprising one or more cationic ionizable lipid(s) is useful for delivery of mRNA, for dendritic cell targeting or methods of using these LNP compositions as a vaccine for the prevention of tuberculosis or other mycobacterial infections. In some embodiments, a LNP can comprise phosphatidylserine or phosphatidylglycerol as targeting ligands to increase their recognition and activity in dendritic cells. In some embodiments, the mRNA is optimized for presentation of MHC-1 epitopes and activation of CD8 T-cell, while in other embodiments the mRNA is optimized for presentation of MHC-II epitopes and activation of CD4 T-cells. In some embodiments the LNP vaccine incorporates both MHC-I and MHC-II optimized mRNA sequences.

Disclosed herein are compounds, compositions and methods related to the treatment of mycobacterial infections. As used herein, the term “compound”, “drug” and “active agent” are used interchangeably. Some aspects of the disclosure relate to novel ionizable lipids or bioreducible ionizable lipids. These lipids are cationic (i.e. positively charged) at acidic pH, such as encountered intracellularly following endocytosis or phagocytosis by a cell. The same lipids, and compositions containing them, are near neutral in charge when present at pH 7.4. These lipids may also have a single olefin group present in their alkyl or acyl groups.

Other aspects relate to compositions comprising lipidic nanoparticles comprising ionizable cationic lipid, the lipidic nanoparticles containing nucleic acids. In some embodiments, nucleic acids are encapsulated into the lipidic nanoparticles.

In some embodiments, a lipid nanoparticle (LNP) composition comprises an ionizable cationic lipid, sterol, one or more phospholipids comprising at least one anionic phospholipid, a conjugated lipid and one or more nucleic acid sequence encoding a T cell epitope from Mycobacterium tuberculosis (Mtb). In some embodiments, a lipid nanoparticle (LNP) composition comprises an ionizable cationic lipid, sterol, one or more phospholipids comprising at least one anionic phospholipid, a conjugated lipid and one or more nucleic acid sequence encoding Mycobacterium tuberculosis antigens recognized by T cells.

Other aspects of the disclosure relate to lipid nanoparticles or targeted lipid nanoparticles that incorporate mRNA coding for major histocompatibility complex class I (MHC-I) or class II (MHC-II) epitopes. In some embodiments, mRNAs coding for MHC-I and MHC-II epitopes are incorporated into a single LNP vaccine. In some embodiments, the epitopes are enriched for those present in Mycobacterium tuberculosis when compared to BCG or nontuberculosis mycobacterium (NTM). In some embodiments, the epitopes in the mRNA cassette are linked with nonimmunogenic linkers. In other embodiments the junctions between epitopes have been optimized to reduce the propensity for forming neoepitopes.

Aspects of the disclosure provide for improved compositions of ionizable lipid nanoparticles for the delivery of therapeutic nucleic acids to cells. Anionic phospholipids, including phosphatidylserine and phosphatidylglycerol are included in the lipid nanoparticles to increase the transfection efficiency in dendritic cells. The further incorporation of ionizable lipids in an LNP formulation with gem di-substitution of mono-unsaturated alkyl chains (single olefin) on 2-position of 1,3-dioxolane or ketal demonstrated high levels of transfection in human dendritic cells, compared to other ionizable lipids in the same family, and demonstrated good stability to oxidative damage.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

As used herein, the following terms and phrases are intended to have the following meanings:

The articles “a” and “an” are used herein to refer to one or to more than one (i.e., to at least one) of the grammatical object of the article. By way of example, “an element” means one element or more than one element.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

The term “comprising” when used in the specification includes “consisting of” and “consisting essentially of”.

If it is referred to “as mentioned above” or “mentioned above”, “supra” within the description it is referred to any of the disclosures made within the specification in any of the preceding pages.

If it is referred to “as mentioned herein”, “described herein”, “provided herein,” or “as mentioned in the present text,” or “stated herein” within the description it is referred to any of the disclosures made within the specification in any of the preceding or subsequent pages.

As used herein, the term “about” means acceptable variations within 20%, within 10% and within 5% of the stated value. In certain embodiments, “about” can mean a variation of +/−1%, 2%, 3%, 4%, 5%, 10% or 20%.

The term “effective amount” as used herein with respect to a compound or the composition means the amount of active compound (also referred herein as active agent or drug) sufficient to cause a bactericidal or bacteriostatic effect. In some embodiments, the effective amount is a “therapeutically effective amount” meaning the amount of active compound that is sufficient alleviate the symptoms of the bacterial infection being treated.

The term “subject” (or, alternatively, “patient”) as used herein refers to an animal, preferably a mammal, most preferably a human that receives either prophylactic or therapeutic treatment.

The term “administration” or “administering” as used herein includes all means of introducing the compounds or the pharmaceutical compositions to the subject in need thereof, including but not limited to, oral, intravenous, intramuscular, intraperitoneal, subcutaneous, transdermal, inhalation, buccal, ocular, sublingual, vaginal, rectal and the like. Administration of the compound or the composition is suitably parenteral. For example, the compounds or the composition can be preferentially administered intravenously, but can also be administered intraperitoneally or via inhalation like is currently used in the clinic for liposomal amikacin in the treatment of Mycobacterium avium (see Shirley et al., Amikacin Liposome Inhalation Suspension: A Review in Mycobacterium avium Complex Lung Disease. Drugs. 2019 April; 79(5):555-562)

The terms “treat,” “treating,” and “treatment,” as used herein, refer to therapeutic or preventative measures such as those described herein.

The term “pharmaceutically acceptable salt” refers to a relatively non-toxic, inorganic or organic acid addition salt of a compound of the present disclosure which salt possesses the desired pharmacological activity.

The term “alkyl” means saturated carbon chains having from one to twenty carbon atoms which may be linear or branched or combinations thereof, unless the carbon chain is defined otherwise. Examples of alkyl groups include methyl, ethyl, propyl, isopropyl, butyl, sec- and tert-butyl, pentyl, hexyl, heptyl, octyl, and the like. Unless stated otherwise specifically in the specification, an alkyl group is optionally substituted.

The term “phosphatidylserine”, with any of it's acyl chain compositions, refers to the L-isomer of serine in the headgroup unless specified in a particular example.

The term “lipid conjugate” refers to a conjugated lipid that inhibits aggregation of lipid particles. Such lipid conjugates include, but are not limited to, polysarcosine (see e.g. WO2021191265A1 which is herein incorporated by reference in its entirety for all purposes), polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides (see, e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

The abbreviations for the ionizable cationic lipids may be truncated in the Examples from that used in the Tables. For example, AKG-UO-1 may be referred to as UO1:

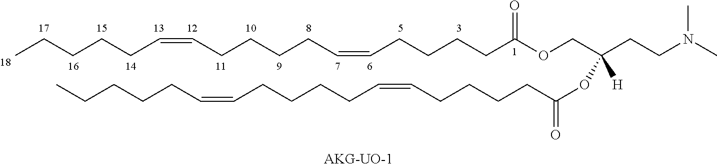 [SMILES: CCCCC/C=C\CCCC/C=C\CCCCC(=O)OC[C@H](CCN(C)C)OC(=O)CCCC/C=C\CCCC/C=C\CCCCC]

For example, AKG-KC2-01 may be referred to as KC2-01.

The abbreviation UT used in various studies refers to untreated samples.

The term “lipidic nanoparticle”, or “LNP”, refers to particles having a diameter of from about 5 to 500 nm. In some embodiments, the lipid nanoparticle comprises one or more active agents. In some embodiments, the lipid nanoparticle comprises a nucleic acid. In some embodiments, the nucleic acid is condensed in the interior of the nanoparticle with a cationic lipid, polymer, or polyvalent small molecule and an external lipid coat that interacts with the biological milieu. Due to the repulsive forces between phosphate groups, nucleic acids are naturally stiff polymers and prefer elongated configurations. In the cell, to cope with volume constraints DNA can pack itself in the appropriate solution conditions with the help of ions and other molecules. Usually, DNA condensation is defined as the collapse of extended DNA chains into compact, orderly particles containing only one or a few molecules. By binding to phosphate groups, cationic lipidic can condense DNA by neutralizing the phosphate charges and allow close packing.

In some embodiments, the active agent is encapsulated into the LNP. In some embodiments, the active agent can be an anionic compounds, for example, but not limited to DNA, RNA, natural and synthetic oligonucleotides (including antisense oligonucleotides, interfering RNA and small interfering RNA), nucleoprotein, peptide, nucleic acid, ribozyme, DNA-containing nucleoprotein, such as an intact or partially deproteinated viral particles (virions), oligomeric and polymeric anionic compounds other than DNA (for example, acid polysaccharides and glycoproteins)). In some embodiments, the active agent can be intermixed with an adjuvant.

In a LNP vaccine product, the active agent is generally contained in the interior of the LNP. In some embodiments, the active agent comprises a nucleic acid. Typically, water soluble nucleic acids are condensed with cationic lipids or polycationic polymers in the interior of the particle and the surface of the particle is enriched in neutral lipids or PEG-lipid derivatives. Additional ionizable cationic lipid may also be at the surface and respond to acidification in the environment by becoming positively charged, facilitating endosomal escape.

Ionizable lipids can have different properties or functions with respect to LNPs. Due to the pKa of the amino group, the lipid molecules can become positively charged in acidic conditions. Under these conditions, lipid molecules can electrostatically bind to the phosphate groups of the nucleic acid which allows the formation of LNPs and the entrapment of the nucleic acid. In some embodiments, the pKa can be low enough that it renders the LNP substantially neutral in surface charge in biological fluids, such as blood, which are at physiological pH values. High LNP surface charge is associated with toxicity, rapid clearance from the circulation by the fixed and free macrophages, hemolytic toxicities, including immune activation (Filion et al Biochim Biophys Acta. 1997 Oct. 23; 1329(2):345-56).

In some embodiments, pKa can be high enough that the ionizable cationic lipid can adopt a positively charged form at acidic endosomal pH values. This way, the cationic lipids can combine with endogenous endosomal anionic lipids to promote membrane lytic nonbilayer structures such as the hexagonal HII phase, resulting in more efficient intracellular delivery. In some embodiments, the pKa ranges between 6.2-7.5. For example, the pKa can be about 6.2, about 6.3, about 6.4, about 6.5, about 6.6, about 6.7, about 6.8, about 6.9, about 7, about 7.1, about 7.2, about 7.3, about 7.4 or about 7.5. Unsaturated tails also contribute to the lipids' ability to adopt nonbilayer structures. (Jayaraman et al., Angew Chem Int Ed Engl. 2012 Aug. 20; 51(34):8529-33).

Release of nucleic acids from LNP formulations, among other characteristics such as liposomal clearance and circulation half-life, can be modified by the presence of polyethylene glycol and/or sterols (e.g. cholesterol) or other potential additives in the LNP, as well as the overall chemical structure, including pKa of any ionizable cationic lipid included as part of the formulation.

The terms “encapsulation” and “entrapped,” as used herein, refer to the incorporation or association of the mRNA, DNA, siRNA or other nucleic acid pharmaceutical agent in or with a lipidic nanoparticle. As used herein, the term “encapsulated” refers to complete encapsulation or partial encapsulation. A siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. A siRNA may comprise a sequence that is complementary to an mRNA sequence that encodes a gene or protein of interest.

The term “mol %” with regard to cholesterol refers to the molar amount of cholesterol relative to the sum of the molar amounts of cholesterol and non-PEGylated phospholipid expressed in percentage points. For example, “55 mol. % cholesterol” in a liposome containing cholesterol and HSPC refers to the composition of 55 mol. parts of cholesterol per 45 mol. parts of HSPC.

The term “mol %” with regard to PEG-lipid refers to the ratio of the molar amount of PEG-lipid and non-PEGylated phospholipid expressed in percentage points. For example, “5 mol. % PEG-DSPE” in a LNP containing HSPC and PEG-DSPE refers to the composition having 5 mol. parts of PEG-DSPE per 100 mol. parts of HSPC.

As used herein, the term “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The term “peptide,” “polypeptide” and “protein” are used interchangeably to denote a sequence polymer of at least two amino acids covalently linked by an amide bond (also referred herein as peptide bond). “Identity,” as known in the art, is a relationship between two or more polypeptide or protein sequences, or nucleic acid sequences as determined by comparing the sequences. In the art, “identity” also refers to the degree of sequence relatedness between polypeptides or proteins, as determined by the match between strings of such sequences. “Identity” can be readily calculated by any bioinformational methods known in the art. “Percent (%) identity” is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity.

The term “substantial identity” or “substantial similarity,” as used herein, when referring to a nucleic acid or fragment thereof, indicates that when optimally aligned with appropriate nucleotide insertions or deletions with another nucleic acid (or its complementary strand), there is nucleotide sequence identity in at least about 95% to 99% of the sequence. The term “substantial identity” or “substantial similarity,” as used herein, when referring to a protein or fragment thereof, indicates that when optimally aligned there is an amino acid sequence identity in at least about 95% to 99% of the sequence.

Various aspects and embodiments are described in further detail in the following subsections.

### Ionizable Cationic Lipids

Provided herein are compounds useful in the preparation of lipid nanoparticle (LNP) compositions.

In some embodiments, a lipid nanoparticle (LNP) composition comprises an ionizable lipid having a chemical structure consisting of a pair of linear polyunsaturated lipid tails covalently bound to a head group, the head group comprising a dialkyl amino group; the head group comprising a heterocyclyl or alkyl portion covalently bound to the dialkyl amino group and optionally further comprising a phosphate group; and each polyunsaturated lipid tail being unsaturated except for at least two olefins separated by at least two methylene groups along the length of the lipid tail, and optionally comprising a single acyl group at the end of the lipid tail covalently bound to the head group. In some aspects, each lipid tail in the ionizable lipid is identical, and each lipid tail has a total of two olefins separated only by an unsubstituted ethylene, n-propyl, or n-butyl. In some embodiments, each lipid tail of the ioniziable lipid further comprises an acyl group joined to an oxygen of the headgroup to form an ester, and has a total of 16 or 18 carbon atoms including the acyl group.

In some embodiments, a lipid nanoparticle (LNP) composition comprises an ionizable cationic lipid having the chemical structure of Formula (I):

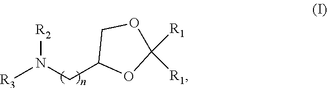

wherein
R₁ is

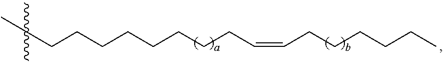

wherein a is 0 or 1; b is 1, 2, 3 or 4, provided the sum a+b is 1, 2, 3 or 4;
R₂ and R₃ are each independently (C₁-C₄)alkyl optionally substituted with hydroxyl; and
n is an integer equal to 2, 3 or 4.

In some embodiments, a lipid nanoparticle (LNP) composition comprises an ionizable cationic lipid having the chemical structure of Formula (I), wherein the total length of the R₁ hydrocarbon chain is C₁₅-C₁₈. In some embodiments, the total length of the R₁ hydrocarbon chain is C₁₆-C₁₈. In some embodiments, the total length of the R₁ hydrocarbon chain is C₁₆ or C₁₈.

In some embodiments, a lipid nanoparticle (LNP) composition comprises an ionizable cationic lipid having the chemical structure of Formula (I), wherein a is 0 and b is 1, 2, 3 or 4. In some embodiments, a lipid nanoparticle (LNP) composition comprises an ionizable cationic lipid having the chemical structure of Formula (I), wherein a is 0 and b is 1 or 3. In some embodiments, a lipid nanoparticle (LNP) composition comprises an ionizable cationic lipid having the chemical structure of Formula (I), wherein a is 0 and b is 1. In some embodiments, a lipid nanoparticle (LNP) composition comprises an ionizable cationic lipid having the chemical structure of Formula (I), wherein a is 0 and b is 3.

In some embodiments, a lipid nanoparticle (LNP) composition comprises an ionizable cationic lipid having the chemical structure of Formula (I), wherein a is 1 and b is 1, 2, 3 or 4. In some embodiments, a lipid nanoparticle (LNP) composition comprises an ionizable cationic lipid having the chemical structure of Formula (I), wherein a is 1 and b is 1 or 3. In some embodiments, a lipid nanoparticle (LNP) composition comprises an ionizable cationic lipid having the chemical structure of Formula (I), wherein a is 1 and b is 1. In some embodiments, a lipid nanoparticle (LNP) composition comprises an ionizable cationic lipid having the chemical structure of Formula (I), wherein a is 1 and b is 3.

In some embodiments, a lipid nanoparticle (LNP) composition comprises an ionizable cationic lipid having the chemical structure of Formula (I), wherein R₁₀ and R₁₂ are the same. In some embodiments, a lipid nanoparticle (LNP) composition comprises an ionizable cationic lipid having the chemical structure of Formula (I), wherein R₁₀ and R₁₂ are each (C₁-C₄)alkyl optionally substituted with hydroxyl. In some embodiments, a lipid nanoparticle (LNP) composition comprises an ionizable cationic lipid having the chemical structure of Formula (I), wherein R₁₀ and R₁₂ are each (C₁-C₄)alkyl. In some embodiments, a lipid nanoparticle (LNP) composition comprises an ionizable cationic lipid having the chemical structure of Formula (I), wherein R₁₀ and R₁₂ are each methyl. In some embodiments, a lipid nanoparticle (LNP) composition comprises an ionizable cationic lipid having the chemical structure of Formula (I), wherein R₁₀ and R₁₂ are each ethyl. In some embodiments, a lipid nanoparticle (LNP) composition comprises an ionizable cationic lipid having the chemical structure of Formula (I), wherein R₁₀ and R₁₂ are each independently selected from methyl or ethyl. In some embodiments, a lipid nanoparticle (LNP) composition comprises an ionizable cationic lipid having the chemical structure of Formula (I), wherein R₁₀ and R₁₂ are each independently selected from methyl, ethyl, —(CH₂)(CH₂)OH, and —(CH₂)₂(CH₂)OH.

In some embodiments, a lipid nanoparticle (LNP) composition comprises an ionizable cationic lipid having the chemical structure of Formula (I), wherein a is 0 or 1; b is 1, 2, 3 or 4, provided the sum a+b is 1, 2, 3 or 4; R₂ and R₃ are each methyl; and n is an integer equal to 2, 3 or 4. In some embodiments, a lipid nanoparticle (LNP) composition comprises an ionizable cationic lipid having the chemical structure of Formula (I), wherein a is 0 or 1; b is 1, 2, 3 or 4, provided the sum a+b is 1, 2, 3 or 4; R₂ and R₃ are each methyl; and n is an integer equal to 2 or 3. In some embodiments, a lipid nanoparticle (LNP) composition comprises an ionizable cationic lipid having the chemical structure of Formula (I), wherein a is 0 or 1; b is 1, 2, 3 or 4, provided the sum a+b is 1, 2, 3 or 4; R₂ and R₃ are each methyl; and n is an integer equal to 2. In some embodiments, a lipid nanoparticle (LNP) composition comprises an ionizable cationic lipid having the chemical structure of Formula (I), wherein a is 0 or 1; b is 1, 2, 3 or 4, provided the sum a+b is 1, 2, 3 or 4; R₂ and R₃ are each methyl; and n is an integer equal to 3.

In some embodiments, an ionizable cationic lipid comprises the chemical structure of Formula (II):

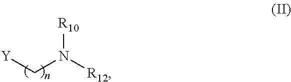

or a pharmaceutically acceptable salt thereof, wherein
Y is,

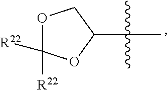

n is an integer 2, 3 or 4;
R²² is a hydrocarbon chain with a single olefin and a total length of C₁₅-C₁₈; and
each of R₁₀ and R₁₂ is independently (C₁-C₄)alkyl optionally substituted with hydroxyl.

In some aspects, R²² in Formula (II) is a polyene hydrorcarbon chain of Formula A.

In some aspects, R₁₀ and R₁₂ in Formula (II) are each independently selected from methyl, ethyl, propyl, —(CH₂)(CH₂)OH, and —(CH₂)₂(CH₂)OH. In some aspects, R₁₀ and R₁₂ are each independently methyl in Formula (II). In some aspects, R₁₀ and R₁₂ are each independently ethyl in Formula (II). In some aspects, at least one of R₁₀ and R₁₂ is n-propyl optionally substituted with hydroxyl in Formula (II). In some aspects, R₁₀ is methyl and R₁₂ is selected from methyl, ethyl, —(CH₂)(CH₂)OH, and —(CH₂)₂(CH₂)OH in Formula (II). In some aspects, R₁₀ is methyl and R₁₂ is selected from —(CH₂)(CH₂)OH, and —(CH₂)₂(CH₂) OH in Formula (II). In some aspects, R₁₀ is methyl and R₁₂ is selected from —(CH₂)(CH₂)OH, and —(CH₂)₂(CH₂)OH in a compound comprising the chemical structure of Formula (II). In some aspects, R₁₀ and R₁₂ are independently selected from methyl or ethyl, optionally substituted with one or more hydroxyl in Formula (II). In some aspects, one or both of R₁₀ and R₁₂ in Formula (II) are —(CH₂)(CH₂)OH, or —(CH₂)₂(CH₂)OH in Formula (II). In some aspects, R₁₀ is methyl and R₁₂ is methyl or ethyl substituted with hydroxyl in Formula (II). In some aspects, one or both of R₁₀ in Formula (II) is methyl and R₁₂ is —(CH₂)(CH₂)OH in Formula (II). In some aspects, one or both of R₁₀ in Formula (II) is methyl and R₁₂ is —(CH₂)₂(CH₂)OH in Formula (II).

In some embodiments, the compounds have the structure of the compounds listed in the tables below. Table 1A show examples of cationic lipids.

TABLE 1A
Exemplary cationic lipids

| | |
|---|---|
| 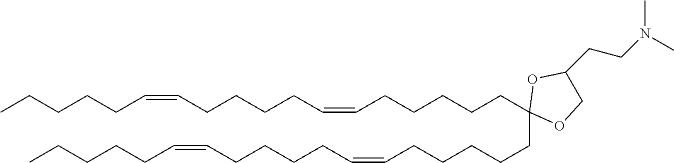 [SMILES: CCCCC/C=C\CCCC/C=C\CCCCCC1(CCCCC/C=C\CCCC/C=C\CCCCC)OCC(CCN(C)C)O1] | 1 |
| 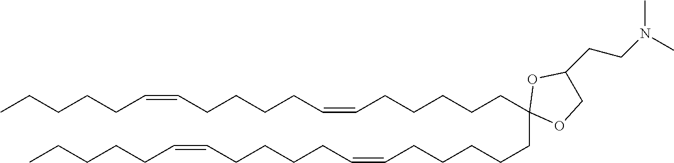 [SMILES: CCCCC/C=C\CCCC/C=C\CCCCCC1(CCCCC/C=C\CCCC/C=C\CCCCC)OCC(CCN(C)C)O1] | 2 |
| 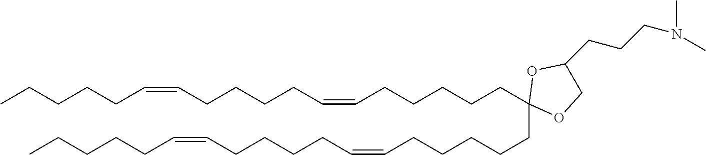 [SMILES: CCCCC/C=C\CCCC/C=C\CCCCCC1(CCCCC/C=C\CCCC/C=C\CCCCC)OCC(CCCN(C)C)O1] | 3 |
| 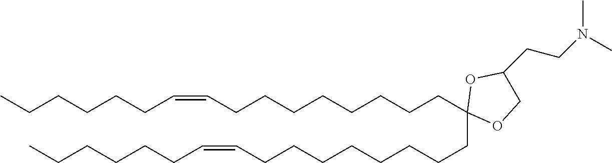 [SMILES: CCCCCC/C=C\CCCCCCCCC1(CCCCCCCC/C=C\CCCCCC)OCC(CCN(C)C)O1] | 4 |
| 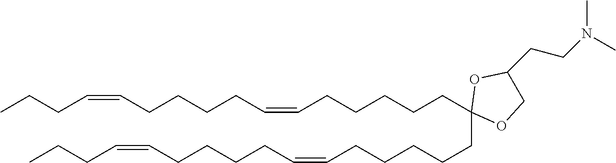 [SMILES: CCC/C=C\CCCC/C=C\CCCCCC1(CCCCC/C=C\CCCC/C=C\CCC)OCC(CCN(C)C)O1] | 5 |
| 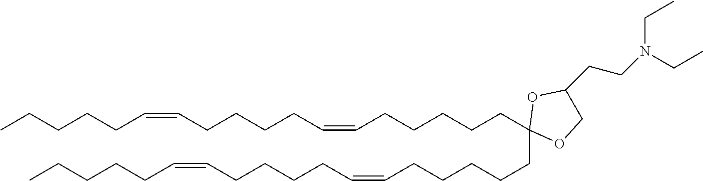 [SMILES: CCCCC/C=C\CCCC/C=C\CCCCCC1(CCCCC/C=C\CCCC/C=C\CCCCC)OCC(CCN(CC)CC)O1] | 6 |
| 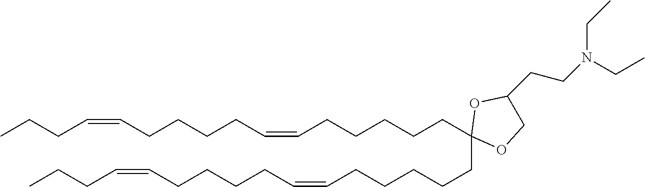 [SMILES: CCC/C=C\CCCC/C=C\CCCCCC1(CCCCC/C=C\CCCC/C=C\CCC)OCC(CCN(CC)CC)O1] | 7 |
| 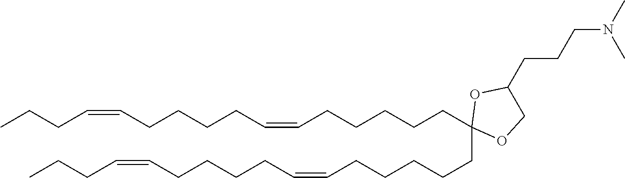 [SMILES: CCC/C=C\CCCC/C=C\CCCCCC1(CCCCC/C=C\CCCC/C=C\CCC)OCC(CCCN(C)C)O1] | 8 |

In some embodiments, the LNP compositions comprises a KC3 ionizable cationic lipid. Unless otherwise indicated, the term “KC3 ionizable cationic lipid” as used herein refers to an ionizable cationic lipid having the chemical formula:

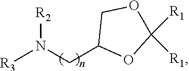

wherein each R₁ is the same or different and is a linear C₁₅ to C₁₉ hydrocarbon chain each comprising one or more unsaturated alkenyl double bond within each polyene hydrocarbon chain; R₂ and R₃ are each independently methyl; and n is 3. In some aspects, each R₁ in the KC3 ionizable cationic lipid is the same or different and is a linear C₁₆ or C₁₈ hydrocarbon chain each comprising one or more unsaturated alkenyl double bond within each polyene hydrocarbon chain.

In some embodiments, the LNP compositions comprises a KC4 ionizable cationic lipid. Unless otherwise indicated, the term “KC4 ionizable cationic lipid” as used herein refers to an ionizable cationic lipid having the chemical formula:

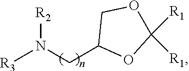

wherein each R₁ is the same or different and is a linear C₁₆ or C₁₈ hydrocarbon chain each comprising one or more unsaturated alkenyl double bond within each polyene hydrocarbon chain; R₂ and R₃ are each independently methyl; and n is 4.

In some aspects, each R₁ in the KC3 or a KC4 ionizable cationic lipid is the same or different and is

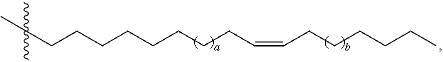

wherein a is 0 or 1; b is 1, 2, 3 or 4, provided the sum a+b is 1, 2, 3 or 4. In some aspects, each R₁ in the KC3 or a KC4 ionizable cationic lipid is the same or different and is

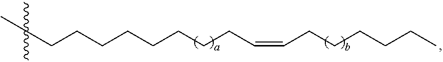

wherein a is 1 and b is 1 or 3. In some aspects, each R₁ in the KC3 or a KC4 ionizable cationic lipid is the same or different and is

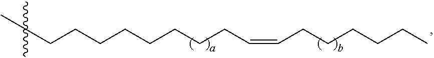

wherein a is 1 and b is 1. In some aspects, each R₁ in the KC3 or a KC4 ionizable cationic lipid is the same or different and is

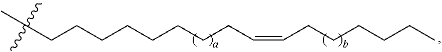

wherein a is 1 and b is 3.

In some aspects, each R₁ in the KC3 or a KC4 ionizable cationic lipid is the same or different and is

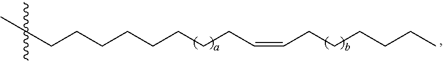

wherein a is 0 and b is 1 or 3. In some aspects, each R₁ in the KC3 or a KC4 ionizable cationic lipid is the same or different and is

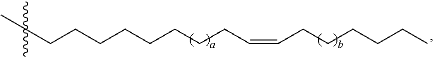

wherein a is 0 and b is 1. In some aspects, each R₁ in the KC3 or a KC4 ionizable cationic lipid is the same or different and is

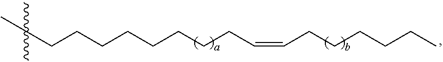

wherein a is 0 and b is 3.

In some aspects, each R₁ in the KC3 or a KC4 ionizable cationic lipid is the same or different and is

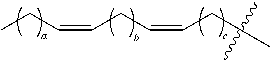

wherein
   a is 1, 2, 3 or 4;
   b is 2, 3 or 4; and
   c is 3, 4, 5, 6, or 7,
provided that the sum of a, b and c is 10, 11, 12 or 13.

In some aspects, each R₁ in the KC3 or a KC4 ionizable cationic lipid is the same or different and is

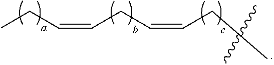

wherein
   a is 1, 2, 3 or 4;
   b is 4; and
   c is 3, 4, 5, 6, or 7,
provided that the sum of a, b and c is 11 or 13.

In some aspects, each R₁ in the KC3 or a KC4 ionizable cationic lipid is the same and is a linear C₁₆ hydrocarbon chain each comprising one unsaturated alkenyl double bond within each polyene hydrocarbon chain. In some aspects, each R₁ in the KC3 or a KC4 ionizable cationic lipid is the same and is a linear C₁₈ hydrocarbon chain each comprising one unsaturated alkenyl double bond within each polyene hydrocarbon chain. In some aspects, each R₁ in the KC3 or a KC4 ionizable cationic lipid is the same and is a linear C₁₆ hydrocarbon chain each comprising two unsaturated alkenyl double bonds within each polyene hydrocarbon chain. In some aspects, each R₁ in the KC3 or a KC4 ionizable cationic lipid is the same and is a linear C₁₆ hydrocarbon chain each comprising two unsaturated alkenyl double bonds within each polyene hydrocarbon chain, wherein the alkenyl double are separated by two or more saturated alkylene groups. In some aspects, each R₁ in the KC3 or a KC4 ionizable cationic lipid is the same and is a linear C₁₈ hydrocarbon chain each comprising one unsaturated alkenyl double bond within each polyene hydrocarbon chain. In some aspects, each R₁ in the KC3 or a KC4 ionizable cationic lipid is the same or different and is a linear C₁₆ or C₁₈ hydrocarbon chain each comprising one or two unsaturated alkenyl double bond within each polyene hydrocarbon chain.

In some embodiments, the LNP compositions comprises a mixture of a KC3 ionizable cationic lipid and a KC4 ionizable cationic lipid.

In some embodiments, the LNP composition comprises an ionizable lipid wherein the ionizable lipid comprises:
(a) the dialkyl amino portion of the head group has a chemical structure of Formula (IV-A)

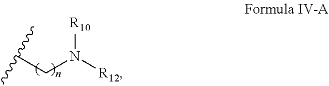

wherein
   n is 2, 3 or 4 in Formula (IV-A); and
   R₁₀ and R₁₂ in Formula (IV-A) are each independently selected from an alkyl group selected from the group consisting of: methyl, ethyl, and propyl, wherein the alkyl in R₁₀ and R₁₂ is optionally substituted with one or more hydroxyl; and
(b) the ionizable lipid further comprises the chemical structure

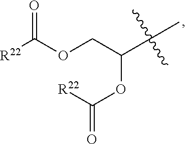

comprising the acyl group of each lipid tail covalently bound to the portion of the head group distal to the dialkyl amino portion of Formula (IV-A), wherein

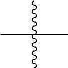

indicates attachment to Formula IV-A within the head group, and R²² indicates a portion of each lipid tail covalently bound to the acyl group and having the chemical structure of Formula A:

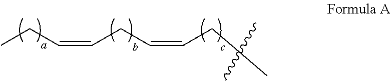

wherein

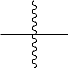

in Formula A indicates attachment of Formula A to R²² within each lipid tail; and
   a is 4, 1, 2, or 3;
   b is 4, 2, or 3; and
   c is 4, 3, 5, 6, or 7,
provided that the sum of a, b and c is in Formula A is 12, 10, 11, or 13.

In some embodiments, the ionizable lipid is a compound of Formula (IV-A), wherein R₁₀ and R₁₂ in Formula (IV-A) are each independently methyl, ethyl, —(CH₂)(CH₂)OH, or —(CH₂)₂(CH₂)OH. In some embodiments, the ionizable lipid is a compound of Formula (IV-A), wherein b is 4 and R₁₀ and R₁₂ in Formula (IV-A) are each methyl.

In some embodiments, the present disclosure provides compositions comprising ionizable cationic lipids. Aspects of the disclosure include compositions comprising 3-rac-2,2-di((Z)-octadec-9-en-1-yl)-1,3-dioxolan-4-yl)-N,N-dimethylpropan-1-amine (AKG-KC3-OA racemate) or chiral purified forms of the AKG-KC3-OA racemate such as KC3-OA(S) and KC3-OA(R), and methods of making and purifying the same. Aspects of the disclosure include compositions comprising 4-rac-2,2-di((Z)-octadec-9-en-1-yl)-1,3-dioxolan-4-yl)-N,N-dimethylbutan-1-amine (AKG-KC4-OA), and methods of making the same. In some embodiments, a composition comprises an ionizable cationic lipid selected from one or more of the following: (a) a racemic mixture of 3-rac-2,2-di((Z)-octadec-9-en-1-yl)-1,3-dioxolan-4-yl)-N,N-dimethylpropan-1-amine (KC3-OA racemate), or KC3-OA enantiomer; and (b) 4-rac-2,2-di((Z)-octadec-9-en-1-yl)-1,3-dioxolan-4-yl)-N,N-dimethylbutan-1-amine (AKG-KC4-OA racemate). In some embodiments, a composition comprises a mixture of (R) and(S) enantiomers of KC3-OA ionizable cationic lipid, or a mixture of (R) and(S) enantiomers of KC4-OA ionizable cationic lipid. In some embodiments, a composition comprises a mixture of (R) and(S) enantiomers of KC3-OA ionizable cationic lipid, or a mixture of (R) and(S) enantiomers of KC4-OA ionizable cationic lipid, and the mixture is racemic.

In some embodiments, the ionizable lipid encapsulate the nucleic acid. In some embodiments, the ionizable lipid encapsulate the nucleic acid in a LNP formulation. In some embodiments, the nucleic acid is a mRNA molecule.

In some embodiments, compositions further comprising ligands, such as antibody conjugates, directed against cell surface receptors to target lipid nanoparticles in a highly specific manner to dendritic cells are provided. In some embodiments, the composition further comprises a targeting ligand, wherein the targeting ligand is oriented to the outside of the nanoparticle. In some embodiments, the targeting ligand is an antibody.

In some embodiments, the lipidic nanoparticles are in an aqueous medium.

In some embodiments, the nucleic acid is entrapped in the lipidic nanoparticle with a compound disclosed herein, including compounds of Formula I, II, III, IV-B, V-A-1 or combinations thereof, wherein the nucleic acid is either RNA. In some embodiments, the nucleic acid is entrapped in the lipidic nanoparticle with a compound disclosed herein, including compounds of disclosed herein or combinations thereof, wherein the nucleic acid is either RNA or DNA. In some embodiments, the nucleic acid is mRNA. In some embodiments, the nucleic acid is siRNA. In some embodiments, the nucleic acid is DNA.

In some embodiments, the lipidic nanoparticle comprises a membrane comprising phosphatidylcholine and a sterol. In some embodiments, the sterol is cholesterol. In some embodiments, the lipidic nanoparticle comprises a membrane comprising phosphatidylcholine, ionizable cationic lipid (ICL). In some embodiments, the ICL have a structure of Formula I, II, III, IV-B, V-A-1, and cholesterol, wherein the membrane separates the inside of the lipidic nanoparticles from the aqueous medium. In some embodiment, the ICL have a structure as shown in Table 1. In some embodiments, the phosphatidylcholine is distearoylphosphatidylcholine (DSPC) or hydrogenated soy phosphatidylcholine (HSPC). In some embodiments, the ionizable cationic lipid to cholesterol molar ratios is from about 65:35 to 40:60. In some embodiments, the ICL to cholesterol molar ratio is from about 60:40 to about 45:55.

In some embodiments, the phosphatidylcholine to cholesterol molar ratio is from about 1:5 to about 1:2.

In some embodiments, the membrane further comprises a polymer-conjugated lipid.

In some embodiments, the lipidic nanoparticle comprises ICL, DSPC, cholesterol and polymer-conjugated lipid in a about 49.5:10.3:39.6:2.5 molar ratio.

In some embodiments, the polymer-conjugated lipid is PEG (2000)-dimyristoylglycerol (PEG-DMG) or PEG (Mol. weight 2,000)-dimyristoylphosphatidylethanolamine (PEG-DMPE).

In some embodiments the percentage of oxidative degradation products for the ionizable lipid is less than 50% of that for a DLin-KC2-DMA or DLin-MC3-DMA control formulation.

In some embodiments, the composition is a liquid pharmaceutical formulation for parenteral administration.

In some embodiments, the composition is a liquid pharmaceutical formulation for subcutaneous, intramuscular, or intradermal administration.

In some embodiments, the composition is in the form of a lyophilized powder, that is subsequently reconstituted with aqueous medium prior to administration.

Other aspects of the disclosure relate to a method of preventing a bacterial or viral infection, the method comprising administering to a subject in need thereof an effective amount of the composition provided herein to elicit an immune response. Some embodiments provide methods of vaccinating a subject in need thereof, the method comprising administering the composition comprising a nucleic acid encoding an antigenic protein.

In some embodiments, the composition is administered subcutaneously, intramuscularly, or intradermally.

In some embodiments, the bacterial infection is Mycobacterium tuberculosis infection. In some embodiments, the bacterial infection is a form of nontuberculosis mycobacterium.

In some embodiments, the lipidic nanoparticle is administered parenterally.

In some embodiments, the lipidic nanoparticle composition is administered as part of a single injection.

In some embodiments the lipid nanoparticle is administered in multiple injections spaced in time to optimize the T-cell response to them.

In some embodiments the lipid nanoparticle is administered intramuscularly (IM).

The present disclosure features a lipid nanoparticle comprising mRNA and lipids. Exemplary lipids include ionizable cationic lipids (ICLs), phospholipids, sterol lipids, alkylene glycol lipids (e.g., polyethylene glycol lipids), sphingolipids, glycerolipids, glycerophospholipids, prenol lipids, saccharolipids, fatty acids, and polyketides. In some embodiments, the LNP comprises a single type of lipid. In some embodiments, the LNP comprises a plurality (e.g. two or more) of lipids. An LNP may comprise one or more of an ionizable cationic lipid, a phospholipid, a sterol, or an alkylene glycol lipid (e.g., a polyethylene glycol lipid).

In an embodiment, the LNP comprises an ionizable cationic lipid. As used herein “ionizable cationic lipid”, “ionizable lipid” and “ICL” are used interchangeably. An ICL is a lipid that comprises an ionizable moiety capable of bearing a charge (e.g., a positive charge e.g., a cationic lipid) under certain conditions (e.g., at a certain pH range, e.g., under physiological conditions). The ionizable moiety may comprise an amine, and preferably a substituted amine. An ionizable lipid may be a cationic lipid or an anionic lipid. In addition to an ionizable moiety, an ionizable lipid may contain an alkyl or alkenyl group, e.g., greater than six carbon atoms in length (e.g., greater than about 8 carbons, 10 carbons, 12 carbons, 14 carbons, 16 carbons, 18 carbons, 20 carbons or more in length). Additional ionizable lipids that may be included in an LNP described herein are disclosed in Jayaraman et al. (Angew. Chem. Int. Ed. 51:8529-8533 (2012)), Semple et al. Nature Biotechnol. 28:172-176 (2010)), and U.S. Pat. Nos. 8,710,200 and 8,754,062, each of which is incorporated herein by reference in its entirety.

In some embodiments, an LNP further comprises an ionizable lipid having a structure of Formula (IV-A), or a pharmaceutically acceptable salt thereof,

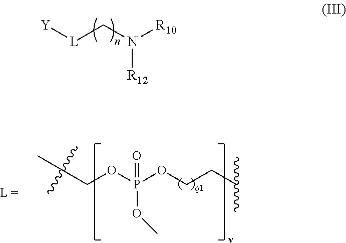

wherein each of R₁₀ and R₁₂ is independently (C₁-C₄)alkyl optionally substituted with hydroxyl; v is 0 or 1; q1 is 1 or 2; Y is

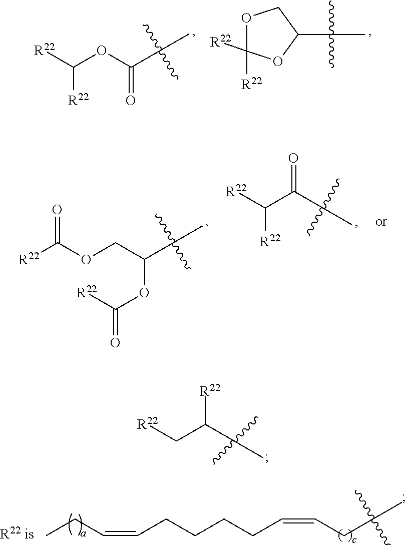

a is 1, 2, 3, 4 or 5; and c is 4, 5, 6, 7 or 8.

In some embodiments, v equals 0 for compounds of Formula (III). In some embodiments, v equals 1 for compounds of Formula (III). In some embodiments, v equals 1 and q1 equals 1 for compounds of Formula (III). In some embodiments, v equals 1 and q1 equals 2 for compounds of Formula (III).

In some embodiments, the sum of a and c is 6, 7, 8 or 9 in R²² for compounds of Formula (III). In some embodiments, the sum of a and c is 6 in R²² for compounds of Formula (III). In some embodiments, the sum of a and c is 7 in R²² for compounds of Formula (III). In some embodiments, the sum of a and c is 9 in R²² for compounds of Formula (III).

In some embodiments, v equals 0 and the sum of a and c is 6, 7, 8 or 9 in R²² for compounds of Formula (III). In some embodiments, v equals 0 and the sum of a and c is 6 in R²² for compounds of Formula (IV-B). In some embodiments, v equals 0 and the sum of a and c is 7 in R²² for compounds of Formula (III). In some embodiments, v equals 0 and the sum of a and c is 9 in R²² for compounds of Formula (III).

In some embodiments, R₁₀ and R₁₂ are independently selected from methyl, ethyl, —(CH₂)(CH₂)OH, and —(CH₂)₂(CH₂)OH for compounds of Formula (III). In some embodiments, R₁₀ and R₁₂ are each methyl and the sum of a and c is 6, 7, 8 or 9 in R²² for compounds of Formula (III). In some embodiments, R₁₀ and R₁₂ are each methyl, vis 0 and the sum of a and c is 6, 7, 8 or 9 in R²² for compounds of Formula (III).

In some embodiments, v equals 0 and R²² is

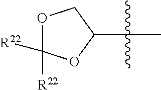

for compounds of Formula (IV-B). In some embodiments, v equals 0 and R²² is

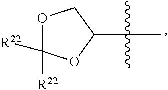

and the sum of a and c is 7 or 9 for compounds of Formula (III). In some embodiments, v equals 0 and R²² is

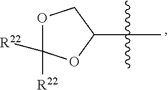

and a is 4 and c is 5 for compounds of Formula (III). In some embodiments, v equals 0 and R²² is

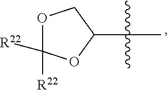

and a is 1 and c is 8 for compounds of Formula (III). In some embodiments, v equals 0 and R²² is

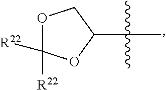

and a is 2 and c is 5 for compounds of Formula (III).

An LNP may comprise an ionizable lipid at a concentration greater than about 0.1 mol %, e.g., of the total lipid content of the LNP. In an embodiment, the LNP comprises an ionizable lipid at a concentration of greater than about 1 mol %, about 2 mol %, about 4 mol %, about 8 mol %, about 20 mol %, about 40 mol %, about 50 mol %, about 60 mol %, about 80 mol %, e.g., of the total lipid content of the LNP. In an embodiment, the LNP comprises an ionizable lipid at a concentration of greater than about 20 mol %, about 40 mol %, or about 50 mol %. In an embodiment, the LNP comprises an ionizable lipid at a concentration between about 1 mol % to about 95 mol %, e.g., of the total lipid content of the LNP. In an embodiment, the LNP comprises an ionizable lipid at a concentration between about 2 mol % to about 90 mol %, about 4 mol % to about 80 mol %, about 10 mol % to about 70 mol %, about 20 mol % to about 60 mol %, about 40 mol % to about 55 mol %, e.g., of the total lipid content of the LNP. In an embodiment, the LNP comprises an ionizable lipid at a concentration between about 20 mol % to about 60 mol %. In an embodiment, the LNP comprises an ionizable lipid at a concentration between about 40 mol % to about 55 mol %.

In an embodiment, the LNP comprises a phospholipid. A phospholipid is a lipid that comprises a phosphate group and at least one alkyl, alkenyl, or heteroalkyl chain. A phospholipid may be naturally occurring or non-naturally occurring (e.g., a synthetic phospholipid). A phospholipid may comprise an amine, amide, ester, carboxyl, choline, hydroxyl, acetal, ether, carbohydrate, sterol, or a glycerol. In some embodiments, a phospholipid may comprise a phosphocholine, phosphosphingolipid, or a plasmalogen. Exemplary phospholipids include 1,2-dioleoyl-sn-glycero-3-phosphocholine (DOPC), 1,2-dipalmitoyl-sn-glycero-3-phosphocholine (DPPC), 1,2-dioleoyl-sn-glycero-3-phosphoethanolamine (DOPE), 1,2-distearoyl-sn-glycero-3-phosphocholine (DSPC), hydrogenated soy phosphatidylcholine (HSPC), 1,2-dilauroyl-sn-glycero-3-phosphocholine (DLPC), 1,2-dimyristoyl-sn-glycero-3-phosphocholine (DMPC), 1,2-distearoyl-sn-glycero-3-phosphoethanolamine (DSPE), 1-myristoyl-2-oleoyl-sn-glycero-3-phosphocholine (MOPC), 1,2-diarachidonoyl-sn-glycero-3-phosphocholine (DAPC), 1-palmitoyl-2-linoleoyl-sn-glycero-3-phosphatidylcholine (PLPC), 1-palmitoyl-2-oleoyl-glycero-3-phosphocholine (POPC), 1-stearoyl-2-myristoyl-sn-glycero-3-phosphocholine (SMPC), 1-palmitoyl-2-myristoyl-sn-glycero-3-phosphocholine (PMPC), bis(monoacylglycerol)phosphate (BMP), L-α-phosphatidylcholine, 1,2-Diheptadecanoyl-sn-glycero-3-phosphorylcholine (DHDPC), and 1-stearoyl-2-arachidonoyl-sn-glycero-3-phosphocholine (SAPC). Additional phospholipids that may be included in an LNP described herein are disclosed in Li, J. et al. (Asian J. Pharm. Sci. 10:81-98(2015)), which is incorporated herein by reference in its entirety.

In some embodiments, the phospholipid is 1,2-distearoyl-sn-glycero-3-phosphocholine some embodiments, the phospholipid is 1,2-dioleoyl-sn-glycero-3-(DSPC). In phosphocholine (DOPC). In some embodiments, the phospholipid is 1,2-dipalmitoyl-sn-glycero-3-phosphocholine (DPPC). In some embodiments, the phospholipid is 1,2-dioleoyl-sn-glycero-3-phosphoethanolamine (DOPE).

### Incorporation of Phosphatidylserine

The LNP (e.g., as described herein) may comprise one or more of the following components: (i) Ionizable cationic lipid (ICL) containing a C16 alkyl or C16 alkenyl group or C18 alkyl or C18 alkenyl group at a concentration between about 1 mol % to about 95 mol % (or any value therebetween, e.g. about 20 mol % to about 80 mol %); (ii) A phospholipid at a concentration between 0.1 mol % to about 20 mol % (or any value there between, e.g. between about 2.5 mol % to about 10 mol %) where the phospholipid also contains C16 or C18 alkyl or alkenyl groups; (iii) cholesterol at a concentration between about 1 mol % to about 95 mol % (or any value therebetween, e.g. about 20 mol % to about 80 mol %); (iv) a phosphatidylserine (PS) or phosphatidylglycerol (PG) added to the LNP lipid formulation at a concentration between about 0.5 mol % to about 20 mol %, about 2.5 mol % to about 10 mol %, about 4 mol % to about 8 mol %, or any value therebetween of the total lipid content of the LNP, and (v) a polyethyleneglycol (PEG)-2000-containing lipid (e.g., DPG-PEG2000, DPPE-PEG2000, DMPE-PEG2000, DMG-PEG2000) at a concentration between about 0.1 mol % to about 5 mol % (or any value therebetween, e.g. between about 1 mol % to about 2.5 mol %). In an embodiment, the LNP comprises two of (i)-(v). In an embodiment, the LNP comprises three of (i)-(v). In an embodiment, the LNP comprises four of (i)-(v). In an embodiment, the LNP comprises each of (i)-(v). In some embodiments, the LNP comprises (i) and (ii). In some embodiments, the LNP comprises (i) and (iii). In some embodiments, the LNP comprises (i) and (v). In some embodiments, the LNP comprises (ii) and (iii). In some embodiments, the LNP comprises (ii) and (v). In some embodiments, the LNP comprises (iii) and (iv). In some embodiments, the LNP comprises (iii) and (v). In some embodiments, the LNP comprises (i), (ii), and (iii). In some embodiments, the LNP comprises (i), (ii), and (v). In some embodiments, the LNP comprises (ii), (iii), and (v). In some embodiments, the LNP comprises (ii), (iii), (iv) and (v). In an embodiment, the LNP consists or consists essentially of four of (i)-(v). In an embodiment, the LNP consists or consists essentially of each of (i)-(v). In some embodiments, the LNP consists or consists essentially of (i) and (ii). In some embodiments, the LNP consists or consists essentially of (i) and (iii). In some embodiments, the LNP consists or consists essentially of (i) and (v). In some embodiments, the LNP consists or consists essentially of (ii) and (iii). In some embodiments, the LNP comprises (ii) and (v). In some embodiments, the LNP consists or consists essentially of (iii) and (iv). In some embodiments, the LNP consists or consists essentially of (iii) and (v). In some embodiments, the LNP consists or consists essentially of (i), (ii), and (iii). In some embodiments, the LNP consists or consists essentially of (i), (ii), and (v). In some embodiments, the LNP comprises (ii), (iii), and (v). In some embodiments, the LNP consists or consists essentially of (ii), (iii), (iv) and (v).

An LNP may comprise a phospholipid at a concentration greater than about 0.1 mol %, e.g., of the total lipid content of the LNP. In an embodiment, the LNP comprises a phospholipid at a concentration of greater than about 0.5 mol %, about 1 mol %, about 1.5 mol %, about 2 mol %, about 3 mol %, about 4 mol %, about 5 mol %, about 6 mol %, about 8 mol %, about 10 mol %, about 12 mol %, about 15 mol %, about 20 mol %, about 50 mol %, e.g., of the total lipid content of the LNP. In an embodiment, the LNP comprises a phospholipid at a concentration of greater than about 1 mol %, about 5 mol %, or about 10 mol %. In an embodiment, the LNP comprises a phospholipid at a concentration between about 0.1 mol % to about 50 mol %, e.g., of the total lipid content of the LNP. In an embodiment, the LNP comprises a phospholipid at a concentration between about 0.5 mol % to about 40 mol %, about 1 mol % to about 30 mol %, about 5 mol % to about 25 mol %, about 10 mol % to about 20 mol %, about 10 mol % to about 15 mol %, or about 15 mol % to about 20 mol %, e.g., of the total lipid content of the LNP. In an embodiment, the LNP comprises a phospholipid at a concentration between about 5 mol % to about 25 mol %. In an embodiment, the LNP comprises a phospholipid at a concentration between about 10 mol % to 20 mol %.

In an embodiment, the LNP comprises a sterol or ionizable sterol molecule. A sterol is a lipid that comprises a polycyclic structure and an optionally a hydroxyl or ether substituent, and may be naturally occurring or non-naturally occurring (e.g., a synthetic sterol). Sterols may comprise no double bonds, a single double bond, or multiple double bonds. Sterols may further comprise an alkyl, alkenyl, halo, ester, ketone, hydroxyl, amine, polyether, carbohydrate, or cyclic moiety. An exemplary listing of sterols includes cholesterol, dehydroergosterol, ergosterol, campesterol, β-sitosterol, stigmasterol, lanosterol, dihydrolanosterol, desmosterol, brassicasterol, lathosterol, zymosterol, 7-dehydrodesmosterol, avenasterol, campestanol, lupeol, and cycloartenol. In some embodiments, the sterol comprises cholesterol, dehydroergosterol, ergosterol, campesterol, β-sitosterol, or stigmasterol. Additional sterols that may be included in an LNP described herein are disclosed in Fahy, E. et al. (J. Lipid. Res. 46:839-862 (2005).

### Ionizable Sterols

In some embodiments, an LNP comprises a sterol. In some embodiments, the sterol is cholesterol. In some embodiments, the sterol is dehydroergosterol. In some embodiments, the sterol is ergosterol. In some embodiments, the sterol is campesterol. In some embodiments, the sterol is β-sitosterol. In some embodiments, the sterol is stigmasterol. In some embodiments, the sterol is a corticosteroid. (e.g., corticosterone, hydrocortisone, cortisone, or aldosterone).

In some embodiments, the ionizable lipid can be a branched ionizable lipid selected from ALC-0315 and SM-102:

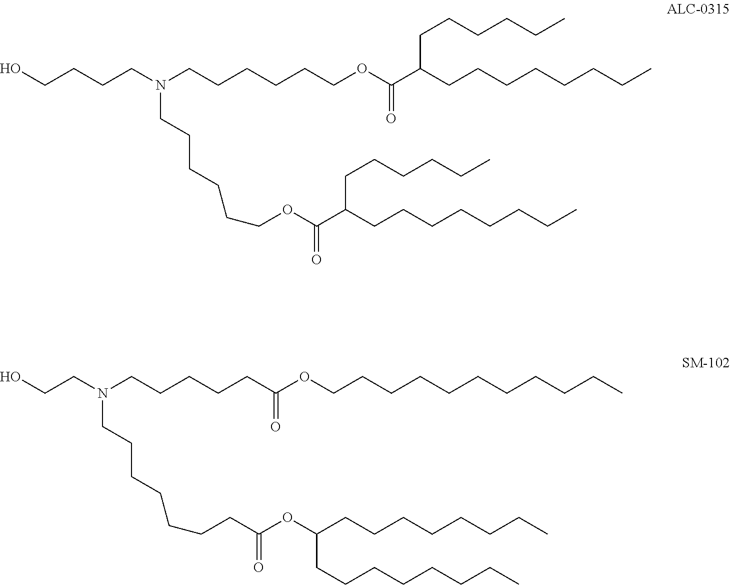 [SMILES: CCCCCCCCC(CCCCCC)C(=O)OCCCCCCN(CCCCO)CCCCCCOC(=O)C(CCCCCC)CCCCCCCC ; CCCCCCCCCCCOC(=O)CCCCCN(CCO)CCCCCCCC(=O)OC(CCCCCCCC)CCCCCCCC]

An LNP may comprise a sterol at a concentration greater than about 0.1 mol %, e.g., of the total lipid content of the LNP. In an embodiment, the LNP comprises a sterol at a concentration greater than about 0.5 mol %, about 1 mol %, about 5 mol %, about 10 mol %, about 15 mol %, about 20 mol %, about 25 mol %, about 35 mol %, about 40 mol %, about 45 mol %, about 50 mol %, about 55 mol %, about 60 mol %, about 65 mol %, or about 70 mol %, e.g., of the total lipid content of the LNP. In an embodiment, the LNP comprises a sterol at a concentration greater than about 10 mol %, about 15 mol %, about 20 mol %, or about 25 mol %. In an embodiment, the LNP comprises a sterol at a concentration between about 1 mol % to about 95 mol %, e.g., of the total lipid content of the LNP. In an embodiment, the LNP comprises a sterol at a concentration between about 5 mol % to about 90 mol %, about 10 mol % to about 85 mol %, about 20 mol % to about 80 mol %, about 20 mol % to about 60 mol %, about 20 mol % to about 50 mol %, or about 20 mol % to 40 mol %, e.g., of the total lipid content of the LNP. In an embodiment, the LNP comprises a sterol at a concentration between about 20 mol % to about 50 mol %. In an embodiment, the LNP comprises a sterol at a concentration between about 30 mol % to about 60 mol %.

In some embodiments, the LNP comprises an alkylene glycol-containing lipid. An alkylene glycol-containing lipid is a lipid that comprises at least one alkylene glycol moiety, for example, a methylene glycol or an ethylene glycol moiety. In some embodiments, the alkylene glycol-containing lipid comprises a polyethylene glycol (PEG). An alkylene glycol-containing lipid may be a PEG-containing lipid. Polymer-conjugated lipids may include poly(ethylene glycol)-conjugated (pegylated)phospholipids (PEG-lipids) such as PEG (Mol. weight 2,000) methoxy-poly(ethylene glycol)-1,2-distearoyl-sn-glycerol (PEG-DSG), PEG (Mol. weight 2,000) methoxy-poly(ethylene glycol)-1,2-palmitoyl-sn-glycerol (PEG-DPG), PEG (Mol. weight 2,000) 1,2-distearoyl-sn-glycero-3-phosphoethanolamine-N-[methoxy(polyethylene glycol)-2000] (PEG-DSPE) or N-palmitoyl-sphingosine-1-{succinyl[methoxy(polyethylene glycol) 2000]} (PEG-ceramide). The molecular weight of the PEG portion in the PEG-lipid component can also vary from 500-10,000 g/mol, from 1,500-6000 g/mol, but is preferably about 2,000 MW. Other polymers used for conjugation to lipid anchors may include poly(2-methyl-2-oxazoline) (PMOZ), poly(2-ethyl-2-oxazoline) (PEOZ), poly-N-vinylpyrrolidone (PVP), polyglycerol, poly(hydroxyethyl L-asparagine) (PHEA), and poly(hydroxyethyl L-glutamine) (PHEG).

A PEG-containing lipid may further comprise an amine, amide, ester, carboxyl, phosphate, choline, hydroxyl, acetal, ether, heterocycle, or carbohydrate. PEG-containing lipids may comprise at least one alkyl or alkenyl group, e.g., greater than six carbon atoms in length (e.g., greater than about 8 carbons, 10 carbons, 12 carbons, 14 carbons, 16 carbons, 18 carbons, 20 carbons or more in length), e.g., in addition to a PEG moiety. In an embodiment, a PEG-containing lipid comprises a PEG moiety comprising at least 20 PEG monomers, e.g., at least 30 PEG monomers, 40 PEG monomers, 45 PEG monomers, 50 PEG monomers, 100 PEG monomers, 200 PEG monomers, 300 PEG monomers, 500 PEG monomers, 1000 PEG monomers, or 2000 PEG monomers. Exemplary PEG-containing lipids include PEG-DMG (e.g., DMG-PEG2k), PEG-c-DMG, PEG-DSG, PEG-DPG, PEG-DSPE, PEG-DMPE, PEG-DPPE, PEG-DOPE, and PEG-DLPE. In some embodiments, the PEG-lipids include PEG-DMG (e.g., DMG-PEG2k), PEG-c-DMG, PEG-DSG, and PEG-DPG. Additional PEG-lipids that may be included in an LNP described herein are disclosed in Fahy, E. et al. (J. Lipid. Res. 46:839-862 (2005) which is incorporated herein by reference in its entirety.

In some embodiments, the PEG-lipid is PEG-DMG (e.g., DMG-PEG2k). In some embodiments, the PEG-lipid is α-(3′-{[1,2-di(myristyloxy)propanoxy]carbonylamino}propyl)-ω-methoxy, polyoxyethylene (PEG-c-DMG). In some embodiments, the PEG-lipid is PEG-DSG. In some embodiments, the PEG-lipid is PEG-DPG.

An LNP may comprise an alkylene glycol-containing lipid at a concentration greater than about 0.1 mol %, e.g., of the total lipid content of the LNP. In an embodiment, the LNP comprises an alkylene glycol-containing lipid at a concentration of greater than about 0.5 mol %, about 1 mol %, about 1.5 mol %, about 2 mol %, about 3 mol %, about 4 mol %, about 5 mol %, about 6 mol %, about 8 mol %, about 10 mol %, about 12 mol %, about 15 mol %, about 20 mol %, about 50 mol %, e.g., of the total lipid content of the LNP. In an embodiment, the LNP comprises an alkylene glycol-containing lipid at a concentration of greater than about 1 mol %, about 4 mol %, or about 6 mol %.

In an embodiment, the LNP comprises an alkylene glycol-containing lipid at a concentration between about 0.1 mol % to about 50 mol %, e.g., of the total lipid content of the LNP. In an embodiment, the LNP comprises an alkylene glycol-containing lipid at a concentration between about 0.5 mol % to about 40 mol %, about 1 mol % to about 35 mol %, about 1.5 mol % to about 30 mol %, about 2 mol % to about 25 mol %, about 2.5 mol % to about 20%, about 3 mol % to about 15 mol %, about 3.5 mol % to about 10 mol %, or about 4 mol % to 9 mol %, e.g., of the total lipid content of the LNP. In an embodiment, the LNP comprises an alkylene glycol-containing lipid at a concentration between about 3.5 mol % to about 10 mol %. In an embodiment, the LNP comprises an alkylene glycol-containing lipid at a concentration between about 4 mol % to 9 mol %.

In some embodiments, the LNP comprises at least two types of lipids. In an embodiment, the LNP comprises two of an ionizable lipid, a phospholipid, a sterol, and an alkylene glycol-containing lipid. In some embodiments, the LNP comprises at least three types of lipids. In an embodiment, the LNP comprises three of an ionizable lipid, a phospholipid, a sterol, and an alkylene glycol-containing lipid. In some embodiments, the LNP comprises at least four types of lipids. In an embodiment, the LNP comprises each of an ionizable lipid, a phospholipid, a sterol, and an alkylene glycol-containing lipid.

The LNP (e.g., as described herein) may comprise one or more of the following components: (i) an ionizable cationic lipid at a concentration between about 1 mol % to about 95 mol % (e.g. about 20 mol % to about 80 mol %); (ii) a phospholipid at a concentration between 0.1 mol % to about 50 mol % (e.g. between about 2.5 mol % to about 20 mol %); (iii) a sterol at a concentration between about 1 mol % to about 95 mol % (e.g. about 20 mol % to about 80 mol %); and (iv) a PEG-containing lipid at a concentration between about 0.1 mol % to about 50 mol % (e.g. between about 2.5 mol % to about 20 mol %). In an embodiment, the LNP comprises one of (i)-(iv). In an embodiment, the LNP comprises two of (i)-(iv). In an embodiment, the LNP comprises three of (i)-(iv). In an embodiment, the LNP comprises each of (i)-(iv). In some embodiments, the LNP comprises (i) and (ii). In some embodiments, the LNP comprises (i) and (iii). In some embodiments, the LNP comprises (i) and (iv). In some embodiments, the LNP comprises (ii) and (iii). In some embodiments, the LNP comprises (ii) and (iv). In some embodiments, the LNP comprises (iii) and (iv). In some embodiments, the LNP comprises (i), (ii), and (iii). In some embodiments, the LNP comprises (i), (ii), and (iv). In some embodiments, the LNP comprises (ii), (iii), and (iv).

The LNP (e.g., as described herein) may comprise one or more of the following components: (i) Ionizable cationic lipid (ICL) at a concentration between about 1 mol % to about 95 mol % (e.g. about 20 mol % to about 80 mol %); (ii) DSPC at a concentration between 0.1 mol % to about 50 mol % (e.g. between about 2.5 mol % to about 20 mol %); (iii) cholesterol at a concentration between about 1 mol % to about 95 mol % (e.g. about 20 mol % to about 80 mol %); and (iv) DMG-PEG2k at a concentration between about 0.1 mol % to about 50 mol % (e.g. between about 2.5 mol % to about 20 mol %). In an embodiment, the LNP comprises two of (i)-(iv). In an embodiment, the LNP comprises three of (i)-(iv). In an embodiment, the LNP comprises each of (i)-(iv). In some embodiments, the LNP comprises (i) and (ii). In some embodiments, the LNP comprises (i) and (iii). In some embodiments, the LNP comprises (i) and (iv). In some embodiments, the LNP comprises (ii) and (iii). In some embodiments, the LNP comprises (ii) and (iv). In some embodiments, the LNP comprises (iii) and (iv). In some embodiments, the LNP comprises (iii) and (iv). In some embodiments, the LNP comprises (i), (ii), and (iii). In some embodiments, the LNP comprises (i), (ii), and (iv). In some embodiments, the LNP comprises (ii), (iii), and (iv).

In an embodiment, the LNP comprises a ratio of ionizable lipid to phospholipid of about 50:1 to about 1:1 (e.g., 40:1, 32:3, 6:1, 7:1, 5:1, 24:5, 26:5, 10:3, 15:2, 16:7, 18:1, 3:1, 3:2, or 1:1). In an embodiment, the LNP comprises a ratio of ionizable lipid to phospholipid of about 15:2. In an embodiment, the LNP comprises a ratio of ionizable lipid to phospholipid of about 5:1. In an embodiment, the LNP comprises a ratio of ionizable lipid to a sterol of about 10:1 to about 1:10 (e.g., 9:1, 8:1, 8:7, 7:1, 7:5, 7:3, 6:1, 6:5, 5:1, 5:3, 4:1, 4:3, 3:1, 2:1, 1:1, 1:2, 1:3, 3:4, 1:4, 3:5, 1:5, 4:5, 1:6, 5:6, 7:6, 7:8, or 8:9). In an embodiment, the LNP comprises a ratio of ionizable lipid to an alkylene-containing lipid of about 1:10 to about 10:1 (e.g., 1:9, 1:8, 7:8, 7:1, 7:5, 7:3, 6:1, 6:5, 5:1, 5:3, 4:1, 4:3, 3:1, 2:1, 1:1, 1:2, 1:3, 3:4, 1:4, 3:5, 1:5, 4:5, 1:6, 5:6, 7:6, 7:8, or 8:9). In an embodiment, the LNP comprises a ratio of phospholipid to an alkylene-containing lipid of about 10:1 to about 1:10 (e.g., 9:1, 8:1, 8:7, 7:1, 7:5, 7:3, 6:1, 6:5, 5:1, 5:3, 4:1, 4:3, 3:1, 2:1, 1:1, 1:2, 1:3, 3:4, 1:4, 3:5, 1:5, 4:5, 1:6, 5:6, 7:6, 7:8, or 8:9). In an embodiment, the LNP comprises a ratio of a sterol to an alkylene-containing lipid of about 50:1 to about 1:1 (e.g., 40:1, 32:3, 6:1, 7:1, 5:1, 24:1, 22:1, 20:1, 22:5, 24:5, 26:5, 10:3, 15:2, 16:7, 18:1, 3:1, 3:2, or 1:1).

In an embodiment, a LNP (e.g., described herein) comprises two of an ionizable lipid, a phospholipid, a sterol, and an alkylene glycol-containing lipid (e.g., PEG-containing lipid). In another embodiment, a LNP (e.g., described herein) comprises three of an ionizable lipid, a phospholipid, a sterol, and an alkylene glycol-containing lipid (e.g., PEG-containing lipid). In an embodiment LNP (e.g., described herein) comprises each of an ionizable lipid, a phospholipid, a sterol, and an alkylene glycol-containing lipid (e.g., PEG-containing lipid).

In some embodiments, an LNP described herein has a diameter between 5 and 500 nm, e.g., between 10 and 400 nm, 20 and 350 nm, 25 and 325 nm, 30 and 300 nm, 50 and 250 nm, 60 and 200 nm, 75 and 190 nm, 80 and 180 nm, 100 and 200 nm, 200 and 300 nm, and 150 and 250 nm. The diameter of an LNP may be determined by any method known in the art, for example, dynamic light scattering, transmission electron microscopy (TEM) or scanning electron microscopy (SEM). In some embodiments, an LNP has a diameter between 50 and 100 nm, between 70 and 100 nm, and between 80 and 100 nm. In an embodiment, an LNP has a diameter of about 90 nm. In some embodiments, an LNP described herein has a diameter greater than about 30 nm. In some embodiments, an LNP has a diameter greater than about 35 nm, about 40 nm, about 45 nm, about 50 nm, about 60 nm, about 70 nm, about 80 nm, about 90 nm, about 100 nm, about 120 nm, about 140 nm, about 160 nm, about 180 nm, about 200 nm, about 225 nm, about 250 nm, about 275 nm or about 300 nm. In an embodiment, an LNP has a diameter greater than about 70 nm. In an embodiment, an LNP has a diameter greater than about 90 nm. In an embodiment, an LNP has a diameter greater than about 180 nm.

In some embodiments, a plurality of LNPs described herein has an average diameter ranging from about 40 nm to about 180 nm. In some embodiments, a plurality of LNPs described herein has an average diameter from about 50 nm to about 150 nm. In some embodiments, a plurality of LNPs described herein has an average diameter from about 50 nm to about 120 nm. In some embodiments, a plurality of LNPs described herein has an average diameter from about 60 nm to about 120 nm. In some embodiments, a plurality of LNPs has an average diameter of about 40 nm, about 45 nm, about 50 nm, about 60 nm, about 70 nm, about 80 nm, about 90 nm, about 100 nm, about 120 nm, about 140 nm, about 160 nm, about 180 nm.

In some embodiments, a nanoparticle or plurality of nanoparticles described herein has an average neutral to negative surface charge of less than −100 mv, for example, less than −90 mv, −80 mv, −70 mv, −60 mv, −50 mv, −40 mv, −30 mv, and −20 mv. In some embodiments, a nanoparticle or plurality of nanoparticles has a neutral to negative surface charge of between −100 mv and 100 mv, between −75 mv to 0, or between −50 mv and −10 mv.

In some embodiments, at least 5% (e.g., at least 10%, at least 15%, at least 20%, at least 25%, at least 30%, at least 40%, at least 50%, at least 60%, at least 70%, at least 80%, at least 90%, at least 95%, or at least 99%) of the nanoparticles of a plurality of nanoparticles have an average neutral to negative surface charge of less than −100 mv. In some embodiments, a nanoparticle or plurality of nanoparticles has an average surface charge of between −20 mv to +20, between −10 mv and +10 mv, or between −5 mv and +5 mv at pH 7.4. LNPs that are neutral in charge have improved pharmacokinetics and biological performance compared to cationic LNPs.

### Making Lipid Nanoparticles (LNPs)

The method of making an LNP can comprise mixing a first solution with a second solution. Mixing can be achieved using standard liquid mixing techniques, such as propellor mixing, vortexing solutions or preferably through microfluidic mixing or high efficiency T-mixing. In some embodiments, the first solution comprises a lipid or a plurality of lipids and a nucleic acid, where all components are solubilized, in water/solvent system. The solvent may be any water miscible solvent (e.g., ethanol, methanol, isopropanol, acetonitrile, dimethylformamide, dimethylsulfoxide, dioxane or tetrahydrofuran). In some embodiments, the first solution comprises a small percentage of water or pH buffered water. The first solution may comprise up to at least 60% by volume of water, e.g., up to at least about 0.05%, 0.1%, 0.5%, 1%, 2%, 3%, 4%, 5%, 10%, 15%, 20%, 25%, 30%, 35%, 40%, 45%, 50%, 55% or 60% by volume of water. In an embodiment, the first solution comprises between about 0.05% and 60% by volume of water, e.g., between about 0.05% and 50%, about 0.05% and 40%, or about 5% and 20% by volume of water.

In some embodiments, the first solution comprises a single type of lipid, for example, an ionizable lipid, a phospholipid, a sterol, or a PEG-containing lipid. In some embodiments, the first solution comprises a plurality of lipids. In some embodiments, the plurality comprises an ionizable lipid, a phospholipid, a sterol, or a PEG-containing lipid. In some embodiments, the plurality of lipids comprise cholesterol, 1,2-distearoyl-sn-glycero-3-phosphocholine (DSPC), 1,2-dimyristoyl-rac-glycero-3-methylpolyoxyethylene2000 (DMG-PEG2k) or α-(3′-{[1,2-di(myristyloxy)propanoxy] carbonylamino}propyl)-ω-methoxy, polyoxyethylene (PEG2000-C-DMG), and an ionizable lipid. The plurality of lipids may exist in any ratio. In an embodiment, the plurality of lipids comprises an ionizable lipid or sterol, a phospholipid, a sterol, a PEG-containing lipid of the above lipids or a combination thereof in a particular ratio (e.g., a ratio described herein).

In some embodiments, the second solution is water. In some embodiments, the second solution is an aqueous buffer with a pH between 3-6 (e.g., a pH of about 3, about 4, about 5, or about 6). The second solution may comprise a load component, e.g., a nucleic acid (e.g., mRNA). The second solution may comprise a small percentage of water-miscible organic solvent. The second solution may comprise up to at least 60% by volume of at least one water miscible organic solvent, e.g., up to at least about 0.05%, 0.1%, 0.5%, 1%, 2%, 3%, 4%, 5%, 6%, 7%, 8%, 9%, 10%, 15%, 20%, 25%, 30%, 35%, 40%, 45%, 50%, 55%, 60% or any percent therebetween by volume of at least one organic solvent (e.g., a water miscible organic solvent). In an embodiment, the second solution comprises between about 0.05% and 60% by volume of organic solvent, e.g., between about 0.05% and 50%, about 0.05% and 40%, or about 5% and 20% by volume of organic solvent (e.g., a water miscible organic solvent). The aqueous buffer solution can be an aqueous solution of citrate buffer. In some embodiments, the aqueous buffer solution is a citrate buffer solution with a pH between 4-6 (e.g., a pH of about 4, about 5, or about 6). In an embodiment, the aqueous buffer solution is a citrate buffer solution with a pH of about 6.

In some embodiments, the solution comprising a mixture of the first and second solutions comprising the LNP suspension can be diluted. In some embodiments, the pH of the solution comprising a mixture of the first and second solutions comprising the LNP suspension can be adjusted. Dilution or adjustment of the pH of the LNP suspension can be achieved with the addition of water, acid, base or aqueous buffer. In some embodiments, no dilution or adjustment of the pH of the LNP suspension is carried out. In some embodiments, both dilution and adjustment of the pH of the LNP suspension is carried out.

In some embodiments, excess reagents, solvents, unencapsulated nucleic acid may be removed from the LNP suspension by tangential flow filtration (TFF) (e.g., diafiltration). The organic solvent (e.g., ethanol) and buffer may also be removed from the LNP suspension with TFF. In some embodiments, the LNP suspension is subjected to dialysis and not TFF. In some embodiments, the LNP suspension is subjected to TFF and not dialysis. In some embodiments, the LNP suspension is subjected to both dialysis and TFF.

In one aspect, the present disclosure features a method comprising treating a sample of LNPs comprising nucleic acid, with a fluid comprising a detergent (e.g., Triton X-100, or anionic detergents (such as, but not limited to, sodium dodecyl sulfate (SDS), or non-ionic detergent, such as but not limited to β-octylglucoside, or Zwittergent 3-14) for a period of time suitable to degrade the lipid layer and thereby release the encapsulated and/or entrapped nucleic acid(s). In an embodiment, the method further comprises analyzing the sample for the presence, absence, and/or amount of the released nucleic acid(s).

### LNP Comprising Ligands

Some aspects of the disclosure relate to LNP comprising a ligand (also referred herein as targeting ligand) having a binding specificity for a cell surface antigen, wherein the binding of the ligand to the antigen induces the internalization of the ligand. Some embodiments relate to compositions comprising LNP comprising a ligand as described herein.

LNP targeting can also accomplished by adding lipids to the formulation. For example, phosphatidylserine is known to redistribute to the external surface of the plasma membrane during apoptosis and is a molecular cue for phagocytotic cell attraction (Fadok et al. Curr Biol. 2003 Aug. 19; 13(16):R₆₅₅-7). Phosphatidylserine (PS) and phosphatidylglycerol (PG) are recognized by dendritic cells and can induce uptake and activation of dendritic cells LNP targeting can also accomplished by adding certain anionic phospholipids to the formulation (Table 2A). For example, phosphatidylserine is known to redistribute to the external surface of the plasma membrane during apoptosis and is a molecular cue for phagocytotic cell attraction (Fadok et al. Curr Biol. 2003 Aug. 19; 13(16):R₆₅₅-7). Phosphatidylserine (PS) and phosphatidylglycerol (PG) are recognized by dendritic cells and can induce uptake and activation of dendritic cells (Caronni et al., Nat Comm. 2021 Apr. 14; 12:2237-2253; Ischihashi et al., PLOS One 2013). Although anionic phospholipids have been used previously in the context of liposomes, their inclusion in lipidic nanoparticles that include condensed nucleic acids is unexpected since anionic headgroups may compete for binding sites of the ionizable cationic lipids with the phosphate backbone of mRNA, may inhibit intracellular escape by altering the surface charge, or may result in aggregation of LNPs during formation or storage.

TABLE 2A
Anionic Phospholipid Targeting Moieties

| | |
|---|---|
| 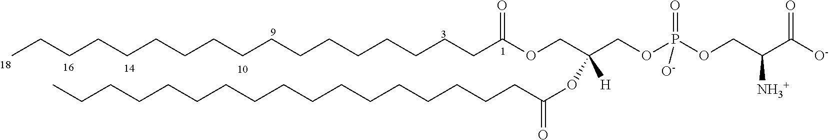 [SMILES: CCCCCCCCCCCCCCCCCC(=O)OC[C@H](COP(=O)([O-])OC[C@H]([NH3+])C(=O)[O-])OC(=O)CCCCCCCCCCCCCCCCC] | DSPS (L-isomer) |
| 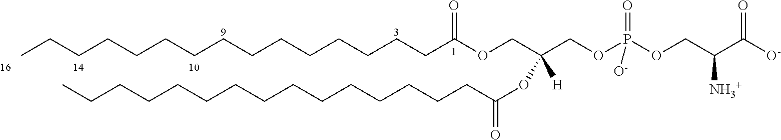 [SMILES: CCCCCCCCCCCCCCCC(=O)OC[C@H](COP(=O)([O-])OC[C@H]([NH3+])C(=O)[O-])OC(=O)CCCCCCCCCCCCCCC] | DPPS (L-isomer) |
| 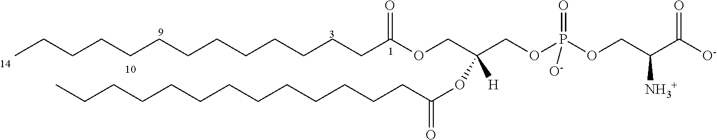 [SMILES: CCCCCCCCCCCCCC(=O)OC[C@H](COP(=O)([O-])OC[C@H]([NH3+])C(=O)[O-])OC(=O)CCCCCCCCCCCCC] | DMPS (L-isomer) |
| 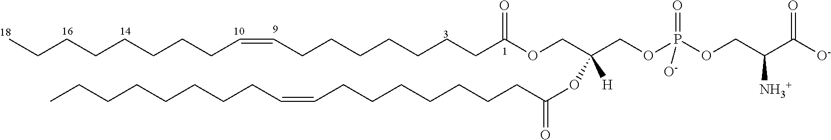 [SMILES: CCCCCCCC/C=C\CCCCCCCC(=O)OC[C@H](COP(=O)([O-])OC[C@H]([NH3+])C(=O)[O-])OC(=O)CCCCCCC/C=C\CCCCCCCC] | DOPS (L-isomer) |
| 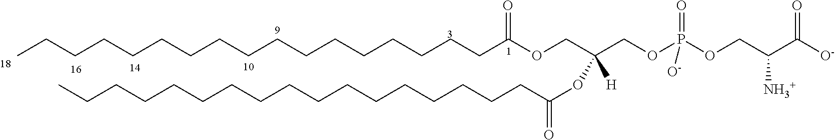 [SMILES: CCCCCCCCCCCCCCCCCC(=O)OC[C@H](COP(=O)([O-])OC[C@@H]([NH3+])C(=O)[O-])OC(=O)CCCCCCCCCCCCCCCCC] | DSPS (D-isomer) |
| 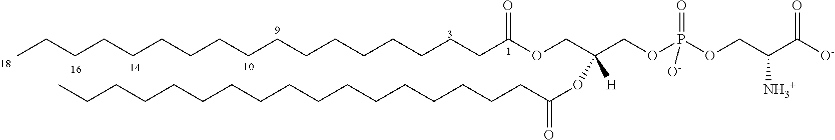 [SMILES: CCCCCCCCCCCCCCCCCC(=O)OC[C@H](COP(=O)([O-])OC[C@@H]([NH3+])C(=O)[O-])OC(=O)CCCCCCCCCCCCCCCCC] | DSPG |
| 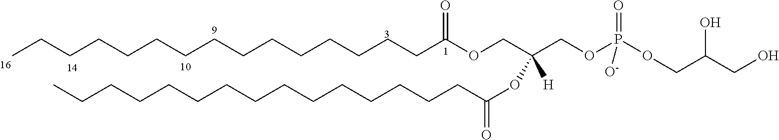 [SMILES: CCCCCCCCCCCCCCCC(=O)OC[C@H](COP(=O)([O-])OCC(O)CO)OC(=O)CCCCCCCCCCCCCCC] | DPPG |
| 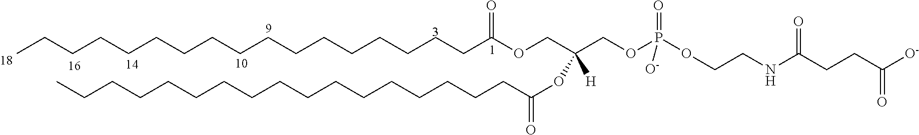 [SMILES: CCCCCCCCCCCCCCCCCC(=O)OC[C@H](COP(=O)([O-])OCCNC(=O)CCC(=O)[O-])OC(=O)CCCCCCCCCCCCCCCCC] | N-Glu-DSPE |
| 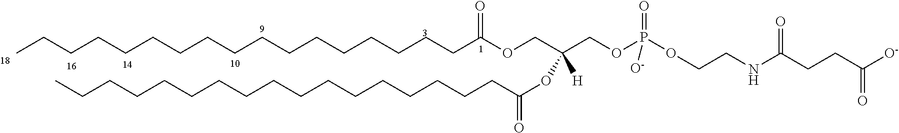 [SMILES: CCCCCCCCCCCCCCCCCC(=O)OC[C@H](COP(=O)([O-])OCCNC(=O)CCC(=O)[O-])OC(=O)CCCCCCCCCCCCCCCCC] | N-Suc-DSPE |

TABLE 2B
Phosphatidylserine Targeting Moieties

| | |
|---|---|
| 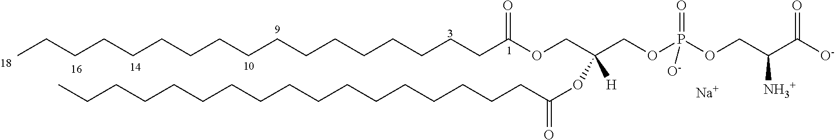 [SMILES: CCCCCCCCCCCCCCCCCC(=O)OC[C@H](COP(=O)([O-])OC[C@H]([NH3+])C(=O)[O-])OC(=O)CCCCCCCCCCCCCCCCC ; [Na+]] | DSPS (L-isomer)- sodium salt |
| 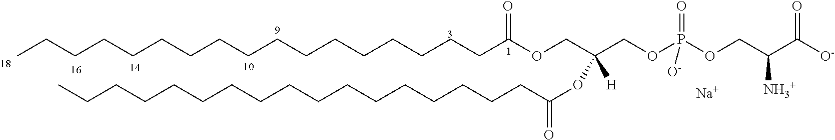 [SMILES: CCCCCCCCCCCCCCCCCC(=O)OC[C@H](COP(=O)([O-])OC[C@H]([NH3+])C(=O)[O-])OC(=O)CCCCCCCCCCCCCCCCC ; [Na+]] | DSPS (L-isomer)- ammonium salt |
| 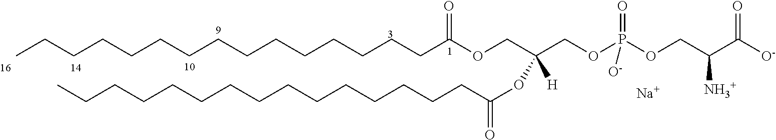 [SMILES: CCCCCCCCCCCCCCCC(=O)OC[C@H](COP(=O)([O-])OC[C@H]([NH3+])C(=O)[O-])OC(=O)CCCCCCCCCCCCCCC ; [Na+]] | DPPS (L-isomer)- sodium salt |
| 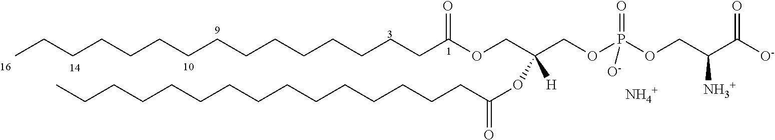 [SMILES: CCCCCCCCCCCCCCCC(=O)OC[C@H](COP(=O)([O-])OC[C@H]([NH3+])C(=O)[O-])OC(=O)CCCCCCCCCCCCCCC ; [NH4+]] | DPPS (L-isomer)- ammonium salt |

In some embodiments, the anionic targeting ligands are selected from the group, phosphatidylserine (PS), phoshatidylglycerol (PG), N-glutaryl-phosphatidylethanolamine (N-glu-PE), or N-succinyl-phosphatidylethanolamine (N-Suc-PE). In some embodiments, the anionic phospholipid used is phosphatidylserine. In another embodiment, the phosphatidylserine contains the L-isomer of serine. In another embodiment, the acyl chains for the phosphatidylserine are fully saturated, such as the case for dimyristoylphosphatidyl-L-serine (DMPS), dipalmitoylphosphatidyl-L-serine (DPPS), or distearoylphosphatidyl-L-serine (DSPS). In a preferred embodiment, the PS used is the L-isomer of either DPPS or DSPS. The phosphatidylserine may also contain an asymmetric acyl chain composition, for example where one acyl chain is stearic acid and another is palmitic acid.

In some embodiments, the anionic phospholipid is selected from a group other than phosphatidylserine. In some embodiments, these non-PS anionic phospholipids include phosphatidylglycerol (PG), phosphatidic acid (PA), N-glutaryl-phosphatidylethanolamine (N-Glu-PE), N-succinyl-phosphatidylethanolamine (N-Suc-PE), and cardiolipin. In some embodiments, these anionic phospholipids include saturated acyl chains of 16 or 18 carbons such as distearoylphosphatidylglycerol (DSPG), dipalmitoyphosphatidylglycerol (DPPG), N-succinyl-distearoylphosphatidylethanolamine (N-Suc-DSPE), N-glutaryl-distearoylphosphatidylethanol-amine (N-Glu-DSPE), distearoylphosphatidic acid (DSPA), and cardiolipin.

TABLE 2C
Nonphosphatidylserine anionic phospholipids

| | |
|---|---|
| 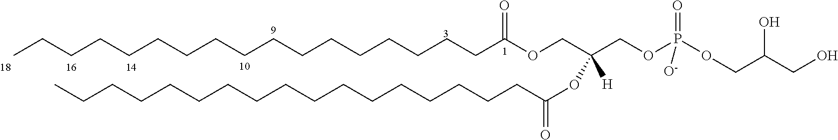 [SMILES: CCCCCCCCCCCCCCCCCC(=O)OC[C@H](COP(=O)([O-])OCC(O)CO)OC(=O)CCCCCCCCCCCCCCCCC] | DSPG |
| 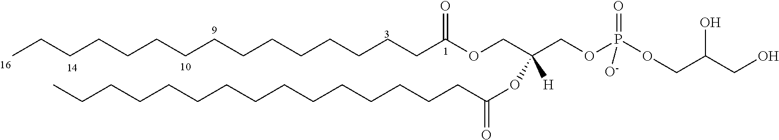 [SMILES: CCCCCCCCCCCCCCCC(=O)OC[C@H](COP(=O)([O-])OCC(O)CO)OC(=O)CCCCCCCCCCCCCCC] | DPPG |
| 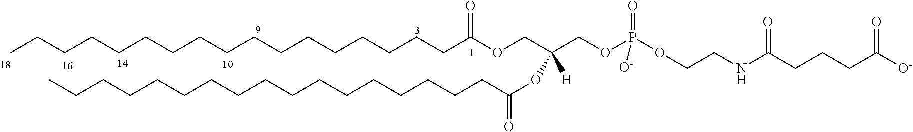 [SMILES: CCCCCCCCCCCCCCCCCC(=O)OC[C@H](COP(=O)([O-])OCCNC(=O)CCCC(=O)[O-])OC(=O)CCCCCCCCCCCCCCCCC] | N-Glu-DSPE |
| 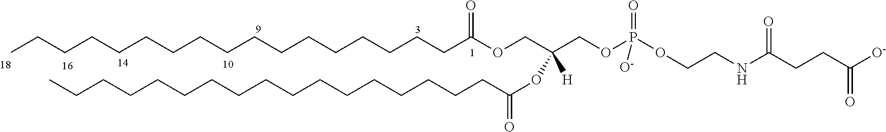 [SMILES: CCCCCCCCCCCCCCCCCC(=O)OC[C@H](COP(=O)([O-])OCCNC(=O)CCC(=O)[O-])OC(=O)CCCCCCCCCCCCCCCCC] | N-Suc-DSPE |
| 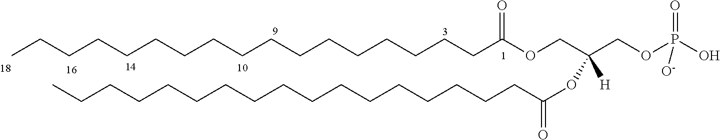 [SMILES: CCCCCCCCCCCCCCCCCC(=O)OC[C@H](COP(=O)([O-])O)OC(=O)CCCCCCCCCCCCCCCCC] | DSPA |
| 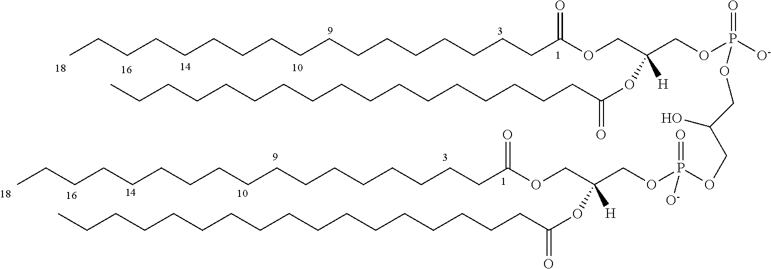 [SMILES: CCCCCCCCCCCCCCCCCC(=O)OC[C@H](COP(=O)([O-])OCC(O)COP(=O)([O-])OC[C@@H](COC(=O)CCCCCCCCCCCCCCCCC)OC(=O)CCCCCCCCCCCCCCCCC)OC(=O)CCCCCCCCCCCCCCCCC] | Cardiolipin |

In some embodiments, the anionic phospholipid used is phosphatidylglycerol. In another embodiment, the acyl chains for the phosphatidylglycerol are fully saturated, such as the case for dimyristoylphosphatidylglycerol (DMPG), dipalmitoylphosphatidylglycerol ((DPPG), or distearoylphosphatidylglycerol (DSPG). In a preferred embodiment, the PG used is either DPPG or DSPG. The phosphatidylglycerol may also contain an asymmetric acyl chain composition, for example where one acyl chain is stearic acid and another is palmitic acid.

In some embodiments, the salt form of phosphatidylglycerol or phosphatidylserine is highly soluble in ethanol. In some embodiments, the salt form of phosphatidylserine is highly soluble in ethanol. In some embodiments it is soluble at greater than 0.5 mg/ml, greater than 1 mg/mL, greater than 5 mg/mL, greater than 10 mg/mL, or greater than 20 mg/mL. In some embodiments the salt form of phosphatidylglycerol or phosphatidylserine is soluble is at least 0.3 mM, at least 0.4 mM, at least 0.5 mM, at least 0.6 mM, or at least 0.8 mM, as determined by a shake flask method in 200 proof ethanol, at the temperature of 22° C. of less. In some embodiments, the salt is an ammonium salt. In some embodiments, the phosphatidylserine is added to the LNP lipids in the form of ammonium or a substituted ammonium salt. Substituted ammonium salt can be mono-, di-. tri-, or tetraalkylammonium having alkyl groups with one to six, one to four, one to three, one, two, or three carbon atoms each. One or more alkyl groups can be n-alkyl, or branched alkyl groups (such as, for example, isopropyl groups), or form a ring (such as for example, cyclohexyl group). An alkyl group and the nitrogen ammonium atom may form a heterocyclic ring. The substituted ammonium salt may be also formed by an alkylenediamine. Tris(hydroxymethyl)aminomethane and triethanolamine can also be used as the amine bases to form PS salts. In some embodiments, the amine is chosen from ammonia, dimethylamine, diethylamine, triethylamine, trimethylamine, 2-(dimethyamino)ethanol, diethanolamine, 2-(diethyamino)ethanol, ethanolamine, ethylenediamine, N-methyl-glucamine, imidazole, histidine, lysine, arginine, 4-(2-hydroxyethyl)-morpholine, piperazine, 1-(2-hydroxyethyl)-pyrrolidine, triethanolamine, and tromethamine (tris(hydroxymethyl)aminomethane), In some embodiments, this targeting lipid is an ammonium salt of DPPS.

TABLE 2D
Ammonium and sodium salt forms of dipalmitoyl- or distearoyl-phosphatidylserine.

| | |
|---|---|
| 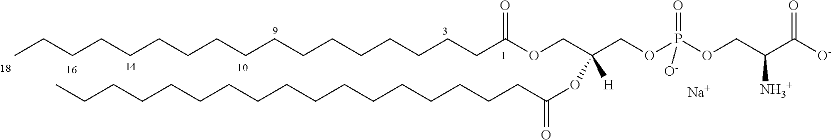 [SMILES: CCCCCCCCCCCCCCCCCC(=O)OC[C@H](COP(=O)([O-])OC[C@H]([NH3+])C(=O)[O-])OC(=O)CCCCCCCCCCCCCCCCC ; [Na+]] | DSPS (-isomer)- sodium salt |
| 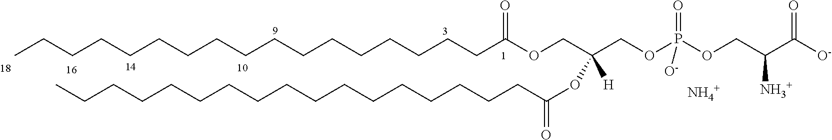 [SMILES: CCCCCCCCCCCCCCCCCC(=O)OC[C@H](COP(=O)([O-])OC[C@H]([NH3+])C(=O)[O-])OC(=O)CCCCCCCCCCCCCCCCC ; [NH4+]] | DSPS (-isomer)- ammonium salt |
| 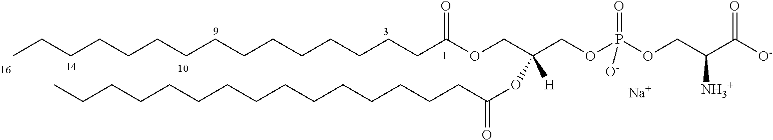 [SMILES: CCCCCCCCCCCCCCCC(=O)OC[C@H](COP(=O)([O-])OC[C@H]([NH3+])C(=O)[O-])OC(=O)CCCCCCCCCCCCCCC ; [Na+]] | DPPS (-isomer)- sodium salt |
| 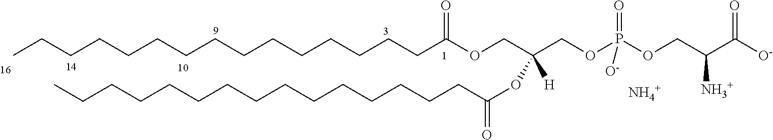 [SMILES: CCCCCCCCCCCCCCCC(=O)OC[C@H](COP(=O)([O-])OC[C@H]([NH3+])C(=O)[O-])OC(=O)CCCCCCCCCCCCCCC ; [NH4+]] | DPPS (Lisomer)- ammonium salt |

To obtain phosphatidylserine in the form of ammonium or substituted ammonium salt, any method known in the art may be used. In some embodiments, a sodium salt of phosphatidylserine (PS) is dissolved in a monophase system of chloroform, methanol, and water, containing a chloride salt of ammonium or substituted ammonium (a Bligh-Dyer monophase), and the system is brought to the two-phase state by adding extra methanol and/or water containing the ammonium or substituted ammonium chloride. The chloroform-rich phase, containing the PS, is separated, and the process is repeated. Finally, the chloroform-rich phase is washed with water to remove excess chloride, and the ammonium (substituted ammonium) salt of PS is obtained by evaporation of the chloroform-rich phase. Optionally, the obtained ammonium or substituted ammonium salt of PS is vacuum dried or dissolved in cyclohexane and lyophilized. In another embodiment, the PS as a sodium or potassium salt is dissolved in a water-immiscible organic solvent, such as chloroform or a chloroform-methanol mixture, and washed with diluted aqueous solution of an acid, such as HCl, to obtain a free acid form of the PS, which is then neutralized with ammonium hydroxide or substituted amine in free base form. In yet another embodiment, the organic solution of PS as a sodium or potassium salt is treated with a cation-exchange resin in the ammonium of substituted ammonium form. In yet another embodiment, the PS is prepared in the form of a calcium or magnesium salt and treated with ammonium or substituted ammonium salt of a chelator, such as EDTA, or with ammonium or substituted ammonium phosphate, in the presence of an organic solvent, causing displacement of calcium or magnesium ion in the form of a chelate or a yet less soluble phosphate, which is separated, e.g., by filtration, while ammonium or substituted ammonium salt of PS is left in the organic (e.g., ethanol) solution.

In some embodiments, PS or PG are added to the LNP lipid formulation at a concentration between about 0.1 mol % to about 20 mol %, about 0.1 mol % to about 10 mol %, about 0.1 mol % to about 5 mol %, about 0.5 mol % to about 20 mol %, about 0.5 mol % to about 10 mol %, about 0.5 mol % to about 5 mol %, about 1 mol % to about 20 mol %, about 1 mol % to about 10 mol %, or about 1 mol % to about 5 mol %, of the total lipid content of the LNP. In some embodiments, the PS is added to the LNP lipid formulation at a concentration between about 1 mol % to about 20 mol %, about 2.5 mol % to about 10 mol %, about 3 mol % to about 9 mol %, or about 4 mol % to about 8 mol %, of the total lipid content of the LNP.

In some embodiments, the PS or PG lipid is included in the LNP composition comprising ionizable cationic lipids known in the art, including DODAP, AKG-OA-DM2, O-11769, DLin-MC3-DMA, DLin-KC2-DMA, DLin-KC3-DMA, ALC-0315, and SM-102.

In another embodiment the PS lipid is included in the LNP composition comprising ICLs of Formula I, II, III, IV-B, V-A-1, combinations thereof or pharmaceutically salts thereof. In another embodiment the PS lipid is included in the LNP composition using N/P ratios between 3 and 8, between 4 and 7, or between 5 and 6.

In some aspects, a method of delivering a nucleic acid to a cell is provided, the method comprising: contacting the cell with a composition comprising an LNP comprising a ligand (also referred herein as targeting ligand) having a binding specificity for a cell surface antigen, wherein the binding of the ligand to the antigen induces the internalization of the ligand. In some embodiments, the targeting ligand can be, but is not limited to, an internalizing antibody, or a fragment thereof, a small molecule conjugates or gylcoconjugates. In some embodiments, the binding of the targeting ligand to a specific cell surface antigen induces the internalization of the LNP with the targeting ligand attached by a cell expressing at least 100,000 or at least 1,000,000 molecules of the antigen when contacted and incubated with the cell under internalizing conditions.

TABLE 3A
Exemplary dialkyl and branched ionizable cationic lipids

| | |
|---|---|
| 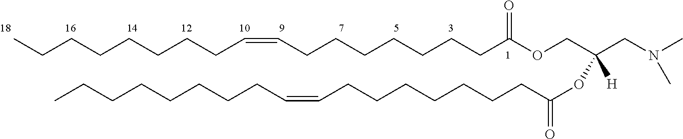 [SMILES: CCCCCCCC/C=C\CCCCCCCC(=O)OC[C@H](CN(C)C)OC(=O)CCCCCCC/C=C\CCCCCCCC] | DODAP |
| 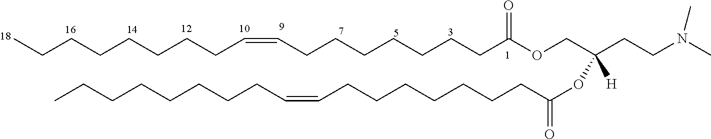 [SMILES: CCCCCCCC/C=C\CCCCCCCC(=O)OC[C@H](CCN(C)C)OC(=O)CCCCCCC/C=C\CCCCCCCC] | AKG-OA-DM2 |
| 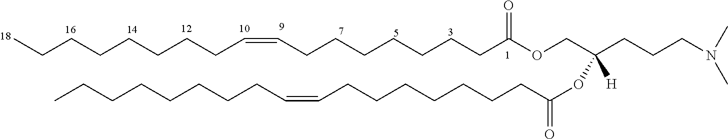 [SMILES: CCCCCCCC/C=C\CCCCCCCC(=O)OC[C@H](CCCN(C)C)OC(=O)CCCCCCC/C=C\CCCCCCCC] | AKG-OA-DM3 |
| 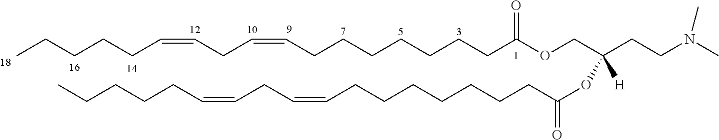 [SMILES: CCCCC/C=C\C/C=C\CCCCCCCC(=O)OC[C@H](CCN(C)C)OC(=O)CCCCCCC/C=C\C/C=C\CCCCC] | O-11769 |
| 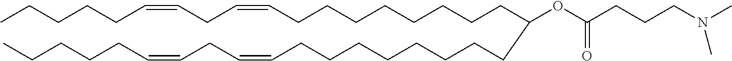 [SMILES: CCCCC/C=C\C/C=C\CCCCCCCCC(CCCCCCCC/C=C\C/C=C\CCCCC)OC(=O)CCCN(C)C] | Dlin-MC3-DMA |
| 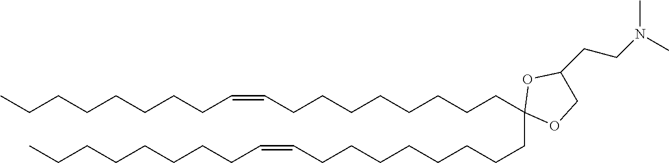 [SMILES: CCCCCCCC/C=C\CCCCCCCCC1(CCCCCCCC/C=C\CCCCCCCC)OCC(CCN(C)C)O1] | AKG-KC2-OA |
| 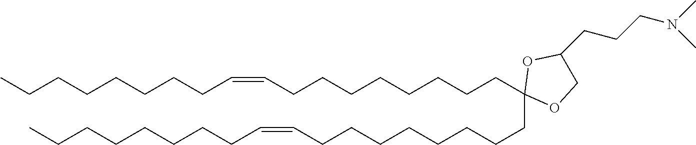 [SMILES: CCCCCCCC/C=C\CCCCCCCCC1(CCCCCCCC/C=C\CCCCCCCC)OCC(CCCN(C)C)O1] | AKG-KC3-OA |
| 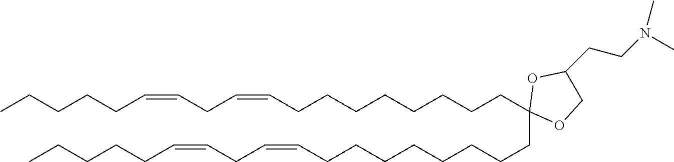 [SMILES: CCCCC/C=C\C/C=C\CCCCCCCCC1(CCCCCCCC/C=C\C/C=C\CCCCC)OCC(CCN(C)C)O1] | Dlin-KC2-DMA |
| 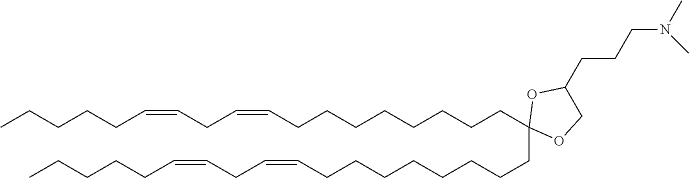 [SMILES: CCCCC/C=C\C/C=C\CCCCCCCCC1(CCCCCCCC/C=C\C/C=C\CCCCC)OCC(CCCN(C)C)O1] | Dlin-KC3-DMA |

TABLE 3B
Exemplary dialkyl and branched ionizable cationic lipids (Continued)

| | |
|---|---|
| 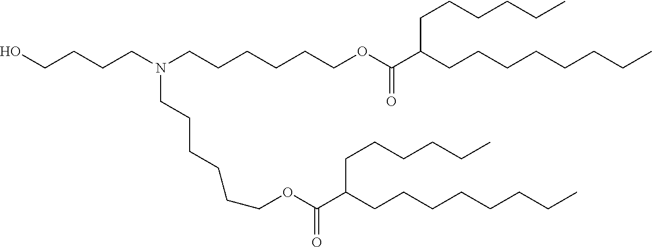 [SMILES: CCCCCCCCC(CCCCCC)C(=O)OCCCCCCN(CCCCO)CCCCCCOC(=O)C(CCCCCC)CCCCCCCC] | ALC-0315 |
| 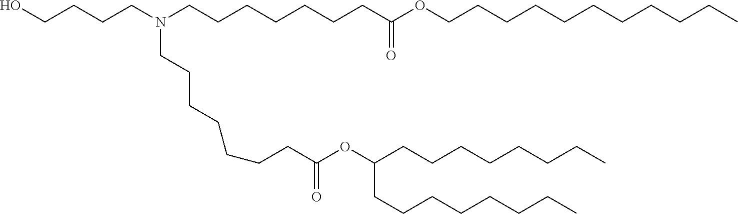 [SMILES: CCCCCCCCCCCOC(=O)CCCCCCCN(CCCCO)CCCCCCCC(=O)OC(CCCCCCCC)CCCCCCCC] | SM-102 |

### Compositions

In some embodiments, a lipidic nanoparticle composition comprises lipids and nucleic acids, the lipidic nanoparticles comprising a compound of Formula I, II, III, IV-B, V-A-1, combinations thereof or pharmaceutically acceptable salts thereof.

Other aspects of the disclosure relate to the use of these ionizable lipids or lipidic nanoparticles compositions comprising ionizable lipids in vaccines for the prevention of infectious diseases. In some embodiments, the compositions described herein can be used to prevent infections related to tuberculosis. In some embodiments, the vaccine is used for the prevention mycobacterium infections. Some embodiments relate to an injectable pharmaceutical vaccine composition comprising a composition of the present disclosure. In some embodiments, the vaccine can be used for the prevention of tuberculosis, nontuberculous mycobacteria (NTM), nontuberculosis lung disease, leprosy, Mycobacterium avium-intracellulare, mycobacterium kansasii, mycobacterium marinum, mycobacterium ulcerans, mycobacterium chelonae, mycobacterium fortuitum, or Mycobacterium abscessus.

In some embodiments, the compounds and compositions described herein promote efficient uptake and transfection of target cells, including tissue macrophages and dendritic cells. The efficient delivery nucleic acids coding for antigen specific for infectious viruses or bacteria, and subsequent presentation of that antigen to elicit the desired immune response to protect against corresponding infections is a result. In some embodiments, the nucleic acid is a synthetic nucleic acid (e.g., engineered codon optimized mRNA) encoding an epitope of Mycobacterium tuberculosis. In some embodiments, the epitopes are MHC class II epitopes included in larger open reading frames (ORFs), such as EsxV (Rv3619), EsxW (Rv3620c), EsxB/CFP10 (Rv3874), EsxA/ESAT-6 (Rv3875), ΔMtb39A (Rv1196), Ag85B (Rv1886c), or AsxH/TB10.4 (Rv0288). In some embodiments, the epitopes are shorter non-overlapping MHC II minimal 15-mer epitopes that were identified in individuals with latent tuberculosis infection (LTBI) (U.S. Pat. No. 10,703,784 which is incorporated herein by reference in its entirety, Arlehamn et al., (2013) PloS Pathog. 9:e1003130, and Arlehamn et al., (2016) PloS Pathog. 12:e1005760). Minimal MHC-II epitopes are defined as the 12-20 residue-long peptide containing the 9-residue core that is the primary determinant of binding strength to the class II molecule binding groove. Due to the open-ended class II binding groove, the flanking residues on either side of the core can vary. In some embodiments, these sequences are concatenated and encoded using a single mRNA. In some embodiments, the concatenated sequence is a combination of the larger open reading frames and the minimal 15-mer epitopes. In some embodiments, the combination of sequences included in a single concatenated sequence is selected to remove redundant protein sequences. In some embodiments the selection of minimal epitopes to be included in the single concatenated sequence is selected to provide optimum HLA donor coverage. In some embodiments the concatenated sequences are joined with nonimmunogenic linkers that reduce the potential for MHC Class II neoepitopes. In some embodiments, the sequence of the peptide linker is comprised of GPGPG (SEQ ID NO: 228).

In some embodiments, the nucleic acid is encoding MHC Class I epitopes. In some embodiments, the Class I epitopes are found in both tuberculosis mycobacterium and other nontuberculosis mycobacterium, or in the Bacillus Calmette-Guerin (BCG) vaccine. In some embodiments, the epitopes are found in tuberculosis mycobacterium. In some embodiments, the MHC Class I sequences are concatenated and encoded using a single mRNA. In some embodiments, the concatenated MHC Class I sequence is a combination of the larger open reading frames and the minimal 9-10-mer epitopes. In other embodiments, the mRNA cassette codes solely for a concatenated sequence of the minimal epitopes. In some embodiments, the combination of sequences included in a single concatenated sequence is selected to remove redundant protein sequences and in some embodiments the selection of minimal epitopes to be included in the single concatenated sequence is selected to provide optimum HLA donor coverage.

In some embodiments, the vaccine candidate includes both an MHC-I and an MHC-II mRNA cassette. In some embodiments, both mRNAs are included in a single targeted LNP preparation. In some embodiments, the MHC-I and MHC-II mRNAs are combined in a 1:1 (wt:wt) ratio (MHC-I/MHC-II). In other embodiments, the mRNAs are included in ratios ranging from about 0.1-to-10 (wt:wt), from about 0.2-to-5 (wt:wt), and from about 0.5-to-2 (wt:wt).

In some embodiments, a lipid nanoparticle (LNP) composition comprises an ionizable cationic lipid, sterol, one or more phospholipids comprising at least one anionic phospholipid, a conjugated lipid and one or more nucleic acid sequence encoding a T cell epitope from Mycobacterium tuberculosis (Mtb). In some embodiments, the nucleic acid sequence encodes a peptide that binds to MHC molecules and is recognized by a T cell receptor (generally 8-11 aa long for MHC class I/CD8 and 12+ for MHC class I/CD4).

In some embodiments, a lipid nanoparticle (LNP) composition comprises an ionizable cationic lipid, sterol, one or more phospholipids comprising at least one anionic phospholipid, a conjugated lipid and one or more nucleic acid sequence encoding Mycobacterium tuberculosis antigens recognized by T cells. In some aspects, the nucleic acid sequence encodes a polypeptide that is recognized by T cells. Peptide fragments can be generated from an antigen that are recognized by T cell receptors.

In some embodiments, a lipid nanoparticle (LNP) composition comprises an ionizable cationic lipid, sterol, one or more phospholipids comprising at least one anionic phospholipid, a conjugated lipid and one or more nucleic acid sequences encoding a Mtb protein selected from the group consisting of: CFP10/Rv3874, ESAT-6/Rv3875, Mtb32A/Rv0125, Mtb39A/Rv1196 and Ag85B/Rv1886c.

In some embodiments, a lipid nanoparticle (LNP) composition comprises an ionizable cationic lipid, sterol, one or more phospholipids comprising at least one anionic phospholipid, a conjugated lipid and one or more nucleic acid sequences selected from the group consisting of: SEQ ID NO:2, SEQ ID NO:3, SEQ ID NO:6, SEQ ID NO:7 and SEQ ID NO:220. In some embodiments, a lipid nanoparticle (LNP) composition comprises an ionizable cationic lipid, sterol, one or more phospholipids comprising at least one anionic phospholipid, a conjugated lipid and a nucleic acid sequence comprising one or more nucleic acid sequences selected from the group consisting of: SEQ ID NO:2, SEQ ID NO:3, SEQ ID NO:6, SEQ ID NO:7 and SEQ ID NO:220.

In some embodiments, a lipid nanoparticle (LNP) composition comprises an ionizable cationic lipid, sterol, one or more phospholipids comprising at least one anionic phospholipid, a conjugated lipid and one or more nucleic acid sequences encoding a Mtb protein selected from the group consisting of: EsxW/Rv3620c, EsxV/Rv3619c, PE13/Rv1195, PPE30/Rv1802, PPE40/Rv2356c, and TB10.4/Rv0288. In some embodiments, a lipid nanoparticle (LNP) composition comprises an ionizable cationic lipid, sterol, one or more phospholipids comprising at least one anionic phospholipid, a conjugated lipid and one or more nucleic acid sequences selected from the group consisting of: SEQ ID NO:1, SEQ ID NO:4, SEQ ID NO:5, SEQ ID NO: 31 SEQ ID NO:221 and SEQ ID NO:222. In some embodiments, a lipid nanoparticle (LNP) composition comprises an ionizable cationic lipid, sterol, one or more phospholipids comprising at least one anionic phospholipid, a conjugated lipid and a nucleic acid sequence comprising one or more nucleic acid sequences selected from the group consisting of: SEQ ID NO:1, SEQ ID NO: 4, SEQ ID NO:5, SEQ ID NO:31 SEQ ID NO:221 and SEQ ID NO:222.

### UTRs

In some concatenated sequences, the untranslated regions (3′ and 5′ UTRs) are chosen to maximize mRNA stability and translation efficiency. UTRs may include those from viral proteins, or human proteins such as hemoglobin alpha (HBA) or hemoglobin beta (HBB) chains.

In some embodiments, chemically modified nucleic acids are incorporated in concatenated mRNA sequences. In some embodiments, the mRNA comprises a modified nucleoside. In some embodiments, the chemically modified residues incorporated are modifications on uridine. In some embodiments, the chemically modified nucleic acid incorporated is pseudouridine. In some embodiments, the chemically modified nucleic acid incorporated is N1-methylpseudouridine (also referred to as 1-methyl-pseudouridine). In some embodiments, the chemically modified nucleic acid incorporated is thiouridine. In some embodiments, the chemically modified nucleic acid incorporated is 5-methylcytidine. In some embodiments, the chemically modified nucleic acid incorporated is 5-methoxyuridine. In some embodiments, the chemically modified nucleic acid incorporated is 5-methylcytidine. In some embodiments, the chemically modified nucleic acid incorporated is N6-methyladenosine. In some embodiments, the chemically modified nucleic acid incorporated is 2′-O-methyluridine, 2-thiouridine.

In some embodiments, the mRNA sequence contains a poly A tail of between about 50-150 nucleotides in length, of between about 80-140 nucleotides in length, of between about 100-140 nucleotides in length. In some embodiments the polyA tail may be interrupted by a short sequence to improve stability.

### Compositions

In some embodiments, the composition further comprises a pharmaceutical excipient.

In some embodiments, the lipidic nanoparticles are in an aqueous medium.

In some embodiments, the nucleic acid is entrapped in the lipidic nanoparticle with an ionizable cationic lipid compound provided herein or combinations thereof, wherein the nucleic acid is either RNA or DNA. In some embodiments, the nucleic acid is mRNA. In some embodiments, the nucleic acid is siRNA. In some embodiments, the nucleic acid is DNA.

In some embodiments, the lipidic nanoparticle comprises a membrane comprising phosphatidylcholine and a sterol. In some embodiments, the sterol is cholesterol. In some embodiments, the lipidic nanoparticle comprises a membrane comprising phosphatidylcholine, ionizable cationic lipid (ICL). In some embodiments, the ICL have a structure of Formula I, and cholesterol, wherein the membrane separates the inside of the lipidic nanoparticles from the aqueous medium. In some embodiment, the ICL have a structure as shown in Table 1A. In some embodiments, the phosphatidylcholine is distearoylphosphatidylcholine (DSPC) or hydrogenated soy phosphatidylcholine (HSPC). In some embodiments, the ionizable cationic lipid to cholesterol molar ratios is from about 65:35 to 40:60. In some embodiments, the ICL to cholesterol molar ratio is from about 60:40 to about 45:55.

In some embodiments, the phosphatidylcholine to cholesterol molar ratio is from about 1:5 to about 1:2.

In some embodiments, the membrane further comprises a polymer-conjugated lipid.

In some embodiments, the lipidic nanoparticle comprises ICL, DSPC, cholesterol and polymer-conjugated lipid in a about 49.5:10.3:39.6:2.5 molar ratio.

In some embodiments, the polymer-conjugated lipid is PEG (2000)-dimyristoylglycerol (PEG-DMG) or PEG (Mol. weight 2,000)-dimyristoylphosphatidylethanolamine (PEG-DMPE).

The compositions of this disclosure may be administered by various routes, for example, to effect systemic delivery via intravenous, parenteral, intraperitoneal, or topical routes. The compositions may be administered intravenously, subcutaneously, or intraperitoneally to a subject. In some embodiments, the disclosure provides methods for in vivo delivery of nucleic acids to a subject.

In some embodiments, the composition is a liquid pharmaceutical formulation for parenteral administration.

In some embodiments, the composition is a liquid pharmaceutical formulation for subcutaneous, intramuscular, or intradermal administration.

In some embodiments, the composition is in the form of a lyophilized powder, that is subsequently reconstituted with aqueous medium prior to administration.

In some embodiments, a lipid nanoparticle (LNP) composition comprises an ionizable cationic lipid having the chemical structure of Formula (I):

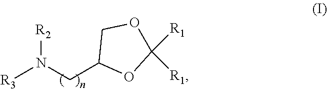

wherein
R₁ is

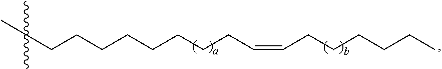

wherein a is 0 or 1; b is 1, 2, 3 or 4, provided the sum a+b is 1, 2, 3 or 4;
R₂ and R₃ are each independently (C₁-C₄)alkyl optionally substituted with hydroxyl; and
n is an integer equal to 2, 3 or 4.
wherein a and b of the two R₁ hydrocarbon chains are the same or different, or one of the two R₁ hydrocarbon chains is a saturated C₁₂-C₁₈ alkyl.

In some embodiments, ionizable cationic lipid compositions are provided. In some embodiments, a lipid nanoparticle (LNP) composition comprises an ionizable cationic lipid having the chemical structure of Formula (I-A):

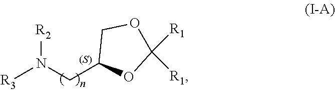

wherein
R₁ is

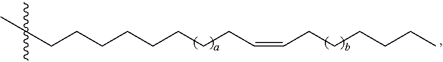

wherein a is 0 or 1; b is 1, 2, 3 or 4, provided the sum a+b is 1, 2, 3 or 4;
R₂ and R₃ are each independently (C₁-C₄)alkyl optionally substituted with hydroxyl; and
n is an integer equal to 2, 3 or 4.

In some embodiments, a and b of the two R₁ hydrocarbon chains are the same.

In some embodiments, a and b of the two R₁ hydrocarbon chains are different.

In some embodiments, one of the two R₁ hydrocarbon chains is a saturated C₁₂-C₁₈ alkyl.

In some embodiments, ionizable cationic lipid compositions are provided. In some embodiments, a lipid nanoparticle (LNP) composition comprises an ionizable cationic lipid having the chemical structure of Formula (I-A):

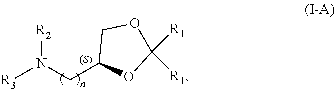

wherein
R₁ is

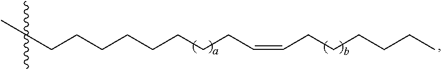

wherein a is 0 or 1; b is 1, 2, 3 or 4, provided the sum a+b is 1, 2, 3 or 4;
R₂ and R₃ are each methyl; and
n is an integer equal to 3.

In some embodiments, a and b of the two R₁ hydrocarbon chains are the same.

In some embodiments, a and b of the two R₁ hydrocarbon chains are different.

In some embodiments, one of the two R₁ hydrocarbon chains is a saturated C₁₂-C₁₈ alkyl.

In some embodiments, ionizable cationic lipid compositions are provided. In some embodiments, a lipid nanoparticle (LNP) composition comprises an ionizable cationic lipid having the chemical structure of Formula (I-A):

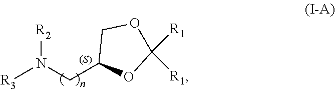

wherein
R₁ is a saturated C₁₅-C₁₈ hydrocarbon chain,
R₂ and R₃ are each methyl; and
n is an integer equal to 3.

In some embodiments, a and b of the two R₁ hydrocarbon chains are the same.

In some embodiments, a and b of the two R₁ hydrocarbon chains are different.

In some embodiments, one of the two R₁ hydrocarbon chains is a saturated C₁₂-C₁₈ alkyl.

In some embodiments, the disclosure provides certain LNP compositions. In some aspects, the LNP compositions comprise: a nucleic acid; an ionizable cationic lipid at a N/P ratio of 3 to 8 relative to the nucleic acid in a total amount of 40-65 mol % of the total lipid content of the LNP composition; a sterol in a total amount of 0.5-50 mol % of the total lipid content of the LNP composition; one or more phospholipids in a total amount of phospholipids of 5-50 mol % of the total lipid content of the LNP composition; and a conjugated lipid in a total amount of 0.5-2.5 mol % of the total lipid content of the LNP composition. In some aspects, the LNP composition is further characterized in that: the nucleic acid is mRNA; the ionizable cationic lipid is present in the LNP composition at a N/P ratio of 4 to 7 relative to the nucleic acid; the sterol is cholesterol; and the conjugated lipid is a PEG-containing conjugated lipid. In some aspects, the one or more phospholipids in the LNP comprise at least two phospholipids having mismatched acyl chain lengths. In some aspects, the one or more phospholipids in the LNP comprise a phosphatidylserine (PS) lipid in a total amount of 2.5-10 mol % of the total lipid content of the LNP composition. In some aspects, the phosphatidylserine (PS) lipid in the LNP consists of, consists essentially of or comprises dipalmitoylphosphatidyl-L-serine ((L-serine) DPPS). In some aspects, the one or more phospholipids in the LNP comprise a phospholipid selected from the group consisting of: distearoylphosphatidylcholine (DSPC) and hydrogenated soy phosphatidylcholine (HSPC). In some aspects, the one or more phospholipids in the LNP consist of distearoylphosphatidylcholine (DSPC) and dipalmitoylphosphatidyl-L-serine ((L-serine) DPPS). In some aspects, the PEG-containing conjugated lipid in the LNP is PEG (2000)-dimyristoylglycerol (PEG-DMG).

In some aspects, the LNP composition has 5-50 mol % total phospholipid, including compositions with 5, 10, 15, 20, 25, 30, 35, 40, 45 or 50 mol % total phospholipid. In some embodiments, the LNP composition is further characterized by: the sterol in a total amount of 0.5-45.5 mol % of the total lipid content of the LNP composition; and the one or more phospholipids in a total amount of phospholipids of 5-50 mol % of the total lipid content of the LNP composition. In some embodiments, the sterol in the LNP composition is cholesterol. In some embodiments, the ionizable cationic lipid is KC3-OA (Racemic). In some embodiments, the ionizable cationic lipid is KC3-OA(S). In some embodiments, the ionizable cationic lipid is KC3-OA(R). In some embodiments, the ionizable cationic lipid is KC4-OA (Racemic). In some embodiments, the ionizable cationic lipid is KC4-OA(S). In some embodiments, the ionizable cationic lipid is KC3-OA(R). In some embodiments, the ionizable cationic lipid is a mixture of KC3-OA and KC4-OA. In some embodiments, the LNP composition comprises a total of 48-54 mol % of the ionizable cationic lipid.

In some embodiments, the one or more phospholipids comprise a phosphatidylserine (PS) lipid. In some embodiments, the PS lipid in the LNP composition is DPPS. In some embodiments, the PS lipid in the LNP composition is present in a total of 5 mol %. In some embodiments, the LNP composition comprises a DSPC phospholipid. In some embodiments, the LNP composition comprises 7.5-20 mol % of a DSPC phospholipid.

In some embodiments, the sterol in the LNP composition is cholesterol. In some embodiments, the LNP composition comprises 25-40 mol % cholesterol. In some embodiments, the sterol in the LNP composition is beta sitosterol. In some embodiments, the LNP composition comprises 33-35.5 mol % beta sitosterol.

In some embodiments, the PEG-containing conjugated lipid in the LNP composition is PEG-DMG. In some embodiments, the LNP composition comprises 1.5-4.0 mol % PEG-containing conjugated lipid in the LNP composition is PEG-DMG. In some embodiments, the PEG-containing conjugated lipid in the LNP composition is PEG-DLG. In some embodiments, the LNP composition comprises 1.0-4.0 mol % PEG-containing conjugated lipid in the LNP composition is PEG-DLG.

In some embodiments, a lipid nanoparticle (LNP) vaccine composition comprises: a nucleic acid; a KC3 ionizable cationic lipid at a N/P ratio of 3 to 7 relative to the nucleic acid in a total amount of 46-54 mol % of the total lipid content of the LNP composition; one or more phospholipids in a total amount of phospholipids of 5-20 mol % of the total lipid content of the LNP composition; a conjugated lipid in a total amount of 1.0-3.5 mol % of the total lipid content of the LNP composition; and cholesterol. In some embodiments, the one or more phospholipids in the LNP comprises an anionic phospholipid in a total of 2-8 mol % of the total lipid content of the LNP composition. In some aspects, the anionic phospholipid is a phosphatidylserine (PS). In some embodiments, the anionic phospholipid is an anionic phospholipid selected from the group consisting of: distearoylphosphatidylglycerol (DSPG) and dipalmitoyphosphatidylglycerol (DPPG). In some embodiments, the anionic phospholipid is an anionic phospholipid selected from the group consisting of: dipalmitoylphosphatidyl-L-serine (DPPS), or distearoylphosphatidyl-L-serine (DSPS).

### Methods of Use

### Targeting of Dendritic Cells

Dendritic cells (DCs) are specialized antigen-presenting cells that play a central role in initiating and regulating adaptive immunity. Owing to their potent antigen (Ag) presentation capacity and ability to generate distinct T-cell responses, efficient and specific delivery of Ags to DCs is the cornerstone for generating Ag-specific effector and memory cells against tumors or pathogens.

Dendritic cells can be generated from human blood monocytes by adding granulocyte-macrophage colony-stimulating factor (GM-CSF), IL-4, and IFN-gamma to differentiate monocyte-derived DC in vitro. Cells in culture exhibit both dendritic and veiled morphologies, the former being adherent, and the latter suspended. Phenotypically, they are CD1a-/dim, CD11a+, CD11b++, CD11c+, CD14dim/−, CD16a−/dim, CD18+, CD32dim/−, CD33+, CD40+, CD45R₀+, CD50+, CD54+, CD64−/dim, CD68+, CD71+, CD80dim, CD86+/++, MHC class I++/, HLA−DR++/, HLA-DP+, and HLA-DQ (Geiseler et al. Dev Immunol. 1998; 6(1-2):25-39).

Alternatively, human primary blood dendritic cell lines have been developed and are commercially available from Creative Biolabs.

CD8+ T cells can produce IL2, IFN-γ, and TNF, cytokines that are known to have critical functions during Mycobacterium tuberculosis infection. Importantly, CD8+ T cells have cytolytic functions to kill Mycobacterium tuberculosis-infected cells via granule-mediated function (via perforin, granzymes, and granulysin) or Fas-Fas ligand interaction to induce apoptosis. In humans, CD8+ T cell can produce granulysin, which can kill Mycobacterium tuberculosis directly. Therefore, it is anticipated that antigen generating mRNA LNPs delivered to DC will stimulate a CD8+ T cell response to fight against Mycobacterium tuberculosis infection.

CD8+ T cells are able to recognize M. tuberculosis specific antigens (as peptides) presented by classical and non-classical MHC molecules. Classically restricted CD8+ T cells have been identified that recognize antigens presented by antigen presenting cells in the context of classical MHC Ia (HLA-A, -B, -C) molecules. Non-classically restricted CD8+ T cells include those CD8+ T cells that are capable of recognizing Mg antigen in the context of HLA-E molecules (non-MHC 1a), glycolipids associated with group 1 CD1 molecules and MHC I-related molecules (MR1) such as mucosal associated invariant T cells (MAIT). Finally, γδ T cells represent a separate population of CD8 (and CD4) T cells that have both innate and adaptive functions in response to Mycobacterium tuberculosis infection. CD8+ T cells have been shown to play direct functions in response to Mycobacterium tuberculosis infection but they also play important roles in orchestrating many different functions in the overall host immune response (e.g., interaction to provide optimal CD4 T cell function)

In some embodiments, LNPs are added to cultured human dendritic cells at an appropriate concentration, (e.g. 1-5 μg/mL mRNA). After some time to allow for cellular uptake and antigen expression, human T cells (HemaCare) can be added, and the cell culture media is sampled at various times for INF-γ by Elisa (R&D Systems, DIF50C). Alternatively, the cells can be analyzed by flow cytometry for CD8+ marker or intracellular INFγ production (PE anti-human IFN-γ antibody, Biolegend).

In some embodiments, LNPs are administered into a subject at a dose of about 0.01 to about 5 mg/kg mRNA by any route of administration known in the art and/or outlined above. According to some embodiments, a proportion of LNPs are taken up DC cells, while most will accumulate in the liver and spleen. The DC cells can express the antigenic peptide, process it for MHC I presentation and travel to the lymph node for presentation to naïve T cells inducing an education of memory T-cells towards the antigen.

In some embodiments, LNPs that have been modified with a targeting ligand such as phosphatidylserine are administered into a subject at a dose of about 1 μg to about 500 μg mRNA. In other embodiments, the targeting ligand is phosphatidylglycerol. In some embodiments the targeted LNPs are administered at a reduced dose of about 1 μg to about 100 ug mRNA. According to some embodiments, a higher proportion of LNPs can be taken up DC cells, allowing for increased production of antigenic peptide compared to non-targeted LNP and a more efficient vaccination against the pathogen. For example, assessing the CD8+ reactivity to the in vivo produced antigen could be accomplished by measuring INFγ plasma levels by species specific IFN-gamma Quantikine ELISA Kits from R&D Systems.

Disclosed herein are methods for preventing mycobacteria infection, such as Mycobacterium tuberculosis. Additional mycobacteria include, but are not limited to, Mycobacterium avium complex, Mycobacterium leprae, Mycobacterium gordonae, Mycobacterium abscessus, Mycobacterium abscessus, Mycobacterium mucogenicum, and Mycobacterium.

Administration of a vaccine for inducing a second immune response may provide MHC class II-presented epitopes that are capable of eliciting a CD4+ helper T cell response against cells expressing antigens from which the MHC presented epitopes are derived. Alternatively or additionally, administration of a vaccine for inducing a second immune response may provide MHC class I-presented epitopes that are capable of eliciting a CD8+ T cell response against cells expressing antigens from which the MHC presented epitopes are derived. Furthermore, administration of a vaccine for inducing a second immune response may provide one or more neo-epitopes (including known neo epitopes) as well as one or more epitopes not containing cancer specific somatic mutations but being expressed by cancer cells and preferably inducing an immune response against cancer cells, preferably a cancer specific immune response. In some embodiments, administration of a vaccine for inducing a second immune response provides neo-epitopes that are MHC class II-presented epitopes and/or are capable of eliciting a CD4+ helper T cell response against cells expressing antigens from which the MHC presented epitopes are derived as well as epitopes not containing cancer—specific somatic mutations that are MHC class I—presented epitopes and/or are capable of eliciting a CD8+ T cell response against cells expressing antigens from which the MHC presented epitopes are derived. In some embodiments, the epitopes do not contain cancer-specific somatic mutations.

As used herein, “cellular immune response”, a “cellular response”, a “cellular response against an antigen” or a similar term are meant to include a cellular response directed to cells characterized by presentation of an antigen with class I or class II MHC. The cellular response relates to cells called T cells or T-lymphocytes which act as either “helper cells” or “killer cells”. The helper T cells (also termed CD4+ T cells) play a central role by regulating the immune response and the killer cells (also termed cytotoxic T cells, cytolytic T cells, CD8+ T cells or CTLS) kill diseased cells such as cancer cells, preventing the production of more diseased cells. In some embodiments, the present disclosure involves the stimulation of an anti-Mycobacterium tuberculosis CTL response against the mycobacterium expressing one or more expressed antigens and preferably presenting such expressed antigens with class I MHC.

An “antigen” according to aspects of the disclosure covers any substance that will elicit an immune response. In particular, an “antigen” relates to any substance, preferably a peptide or protein, that reacts specifically with antibodies or T-lymphocytes (T cells). As used herein, the term “antigen” comprises any molecule which comprises at least one epitope. Preferably, an antigen in the context of the present disclosure is a molecule which, optionally after processing, induces an immune reaction, which is preferably specific for the antigen (including cells expressing the antigen). According to aspects of the present disclosure, any suitable antigen may be used, which is a candidate for an immune reaction, wherein the immune reaction is preferably a cellular immune reaction. In the context of the embodiments of the present disclosure, the antigen is presented by a cell, for example by an antigen presenting cell which includes a diseased cell, in particular a cancer cell, in the context of MHC molecules, which results in an immune reaction against the antigen. An antigen can be a product which corresponds to or is derived from a naturally occurring antigen. Such naturally occurring antigens may include tumor antigens.

As used herein, an “antigen peptide” refers to a portion or fragment of an antigen which is capable of stimulating an immune response, preferably a cellular response against the antigen or cells characterized by expression of the antigen and preferably by presentation of the antigen such as diseased cells, in particular cancer cells. Preferably, an antigen peptide is capable of stimulating a cellular response against a cell characterized by presentation of an antigen with class I MHC and preferably is capable of stimulating an antigen-responsive cytotoxic T-lymphocyte (CTL). The antigen peptides according to embodiments are MHC class I and/or class II presented peptides or can be processed to produce MHC class I and/or class II presented peptides. In some embodiments, the antigen peptides comprise an amino acid sequence substantially corresponding to the amino acid sequence of a fragment of an antigen. In some embodiments, said fragment of an antigen is an MHC class I and/or class II presented peptide. In some embodiments, an antigen peptide comprises an amino acid sequence substantially corresponding to the amino acid sequence of such fragment and is processed to produce such fragment, i.e., an MHC class I and/or class II presented peptide derived from an antigen. According to some embodiments, if a peptide is to be presented directly, i.e., without processing, in particular without cleavage, the peptide has a length which is suitable for binding to an MHC molecule, in particular a class I MHC molecule. In some embodiments, the peptide has a length of 7-20 amino acids, 7-12 amino acids, 8-11 amino acids, for example 7, 8, 9, 10, 11, 12, 13, 14, 15, 16, 17, 18, 19, 20 amino acids in length.

The main types of professional antigen-presenting cells are dendritic cells, which have the broadest range of antigen presentation, and are probably the most important antigen-presenting cells, macrophages, B-cells, and certain activated epithelial cells. Dendritic cells (DCs) are leukocyte populations that present antigens captured in peripheral tissues to T cells via both MHC class II and I antigen presentation pathways. It is well known that dendritic cells are potent inducers of immune responses and the activation of these cells is a critical step for the induction of antitumoral immunity. Dendritic cells are conveniently categorized as “immature” and “mature” cells, which can be used as a simple way to discriminate between two well characterized phenotypes.

However, this nomenclature should not be construed to exclude all possible intermediate stages of differentiation. Immature dendritic cells are characterized as antigen presenting cells with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g. CD54 and CD11) and costimulatory molecules (e.g., CD40, CD80, CD86 and 4-1 BB). Dendritic cell maturation is referred to as the status of dendritic cell activation at which such antigen-presenting dendritic cells lead to T cell priming, while presentation by immature dendritic cells results in tolerance. Dendritic cell maturation is chiefly caused by biomolecules with microbial features detected by innate receptors (bacterial DNA, viral RNA, endotoxin, etc), pro-inflammatory cytokines (TNF, IL-1, IFNs), ligation of CD40 on the dendritic cell surface by CD40L, and substances released from cells undergoing stressful cell death. The dendritic cells can be derived by culturing bone marrow cells in vitro with cytokines, such as granulocyte-macrophage colony-stimulating factor (GM CSF) and tumor necrosis factor alpha. Non-professional antigen-presenting cells do not constitutively express the MHC class II proteins required for interaction with naive T cells; these are expressed only upon stimulation of the non-professional antigen-presenting cells by certain cytokines such as IFNγ. “Antigen presenting cells” can be loaded with MHC class I presented peptides by transducing the cells with nucleic acid, preferably mRNA, encoding a peptide or polypeptide comprising the peptide to be presented, e.g. a nucleic acid encoding the antigen.

In some embodiments, a pharmaceutical composition comprising a gene delivery vehicle that targets a dendritic or other antigen presenting cell is administered to a patient, resulting in transfection that occurs in vivo. As used herein, a “nucleic acid” refers to a deoxyribonucleic acid (DNA) or ribonucleic acid (RNA). In some embodiments, the nucleic acid is an RNA, for example an in vitro transcribed RNA (IVT RNA) or synthetic RNA. Nucleic acids include according to aspects of the disclosure genomic DNA, cDNA, mRNA, recombinantly produced and chemically synthesized molecules. According to aspects of the disclosure, a nucleic acid may be present as a single-stranded or double-stranded and linear or covalently circularly closed molecule. A nucleic acid can, according to aspects of the disclosure, be isolated. In some embodiments, the nucleic acid (i) was amplified in vitro, for example via polymerase chain reaction (PCR), (ii) was produced recombinantly by cloning, (iii) was purified, for example, by cleavage and separation by gel electrophoresis, or (iv) was synthesized, for example, by chemical synthesis. A nucleic can be employed for introduction into, i.e. transfection of cells, in particular, in the form of RNA which can be prepared by in vitro transcription from a DNA template. The RNA can moreover be modified before application by stabilizing sequences, capping, and polyadenylation.

As used herein, the term “RNA” refers to a molecule which comprises ribonucleotide residues and preferably being entirely or substantially composed of ribonucleotide residues. As used herein, the term “ribonucleotide” refers to a nucleotide with a hydroxyl group at the 2′-position of a B-D-ribofuranosyl group. As used herein, the term “RNA” comprises double-stranded RNA, single-stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally occurring RNA.

In some embodiments, the RNA is a mRNA. As used herein, the term “mRNA” means “messenger RNA” and refers to a “transcript” which can be generated by using a DNA template and encodes a peptide or polypeptide. Typically, an mRNA comprises a 5′-UTR, a protein coding region, and a 3′-UTR. mRNA only possesses limited half-life in cells and in vitro. In the context of aspects of the present disclosure, mRNA may be generated by in vitro transcription from a DNA template. As used herein, the term “modification” in the context of the RNA used in aspects of the disclosure includes any modification of an RNA which is not naturally present in said RNA. According to some embodiments, the RNA does not have uncapped 5′-triphosphates. Removal of such uncapped 5′-triphosphates can be achieved by treating RNA with a phosphatase. The RNA according to aspects of the disclosure may have modified ribonucleotides in order to increase its stability and/or decrease cytotoxicity. For example, in some embodiment, 5-methylcytidine in the RNA is substituted partially or completely, for cytidine. In some embodiments, 5-methylcytidine in the RNA is substituted completely for cytidine. Alternatively or additionally, in some embodiments, pseudouridine in the RNA used is substituted partially or completely, for uridine. In some embodiments, pseudouridine in the RNA used is substituted completely for uridine.

In some embodiments, the RNA can be provided with a 5-cap or 5′-cap analog. The term “5-cap” refers to a cap structure found on the 5′-end of an mRNA molecule and generally consists of a guanosine nucleotide connected to the mRNA via an unusual 5′ to 5 triphosphate linkage. In some embodiments, this guanosine is methylated at the 7-position. The term “conventional 5′-cap” refers to a naturally occurring RNA 5 ‘-cap, for example to the 7-methylguanosine cap (m′G). In some embodiments, the 5’-cap includes a 5′-cap analog that resembles the RNA cap structure and is modified to possess the ability to stabilize RNA and/or enhance translation of RNA if attached thereto, preferably in vivo and/or in a cell.

According to aspects of the disclosure, the stability and translation efficiency of RNA may be modified as required. For example, RNA may be stabilized and its translation increased by one or more modifications having a stabilizing effects and/or increasing translation efficiency of RNA. Such modifications are described, for example, in PCT/EP2006/009448 incorporated herein by reference in its entirety. In order to increase expression of the RNA used according to aspects of the present disclosure, it may be modified within the coding region, i.e. the sequence encoding the expressed peptide or protein, preferably without altering the sequence of the expressed peptide or protein, so as to increase the GC content to increase mRNA stability and to perform a codon optimization and, thus, enhance translation in cells.

Lipid nanoparticle (LNP) compositions are provided herein, and methods of making and using the same. In some embodiments, the LNP compositions comprise a nucleic acid such as messenger ribonucleic acid (mRNA). In some embodiments, the LNP compositions are vaccines, including LNP formulations comprising mRNA that encodes an immune system epitope, or an antigen recognized by the immune system.

In some aspects, the LNP comprises nucleic acid containing a chemically modified mRNA, wherein the chemically modified mRNA comprises N1-methylpseudouridine. In some aspects, the LNP comprises nucleic acid comprising a 5′ untranslated region (UTR) and 3′ UTR, polyA tail of about 80 to about 140 nucleotides in length, and (i) a 5′ enzymatic or (ii) a 5′ clean cap. In some aspects, the LNP comprises a chemically modified mRNA, wherein the chemically modified mRNA comprises N1-methylpseudouridine

In some embodiments, the LNP composition comprises: (a) a nucleic acid; (b) an ionizable cationic lipid comprising a KC3 ionizable cationic lipid at a N/P ratio of 3 to 7 relative to the nucleic acid, the ionizable cationic lipid present in the LNP composition in a total amount of 46-54 mol % of a total lipid content of the LNP composition; (c) one or more phospholipids in a total amount of 5-20 mol % of the total lipid content of the LNP composition; (d) one or more anionic phospholipids in a total amount of 2-8 mol % of the total lipid content of the LNP composition; (e) a conjugated lipid in a total amount of 1-3.5 mol % of the total lipid content of the LNP composition; and (f) a sterol such cholesterol (e.g., in an amount providing the remainder of the LNP composition). In some aspects, the one or more anionic phospholipids is a phosphatidylserine (PS) or phosphatidylglycerol (PG). In some aspects, the one or more anionic phospholipids is selected from the group consisting of: dipalmitoylphosphatidyl-L-serine (DPPS), or distearoylphosphatidyl-L-serine (DSPS), distearoylphosphatidylglycerol (DSPG), and dipalmitoyphosphatidylglycerol (DPPG). In some aspects, the one or more phospholipids comprises distearoylphosphatidylcholine (DSPC), hydrogenated soy phosphatidylcholine (HSPC), dipalmitoylphosphatidylcholine (DPPC) or a combination thereof. In some aspects, the conjugated lipid is PEG (2000)-dimyristoylglycerol (PEG-DMG). In some aspects, the sterol is cholesterol. In some aspects, the ionizable cationic lipid comprises 3-((S)-2,2-di((Z)-octadec-9-en-1-yl)-1,3-dioxolan-4-yl)-N,N-dimethylpropan-1-amine (KC3-OA). In some aspects, the ionizable cationic lipid further comprises a KC4 ionizable cationic lipid, such as 4-rac-2,2-di((Z)-octadec-9-en-1-yl)-1,3-dioxolan-4-yl)-N,N-dimethylbutan-1-amine (AKG-KC4-OA). In some aspects, the LNP composition consists of: 48 mol % KC3-OA; 5 mol % DPPS or DSPG; 5-10 mol % DSPC or HSPC; 1.5 mol % PEG-DMG; and cholesterol. In some aspects, the LNP composition consists of: 48 mol % KC3-OA; 5 mol % DPPS or DSPG; 5 mol % DSPC or HSPC; 1.5 mol % PEG-DMG; and 40.5 mol % cholesterol. In some aspects, the LNP composition consists of: 48 mol % KC3-OA; 5 mol % DPPS or DSPG; 10 mol % DSPC or HSPC; 1.5 mol % PEG-DMG; and 35.5 mol % cholesterol.

In some embodiments, a method of eliciting a T cell response in a host is provided, comprising administering to the host a nucleic acid sequence disclosed herein or a nucleic acid having at least 90% sequence identity or complementarity to a sequence disclosed herein, and/or a sequence encoding a T cell epitope from Mycobacterium tuberculosis (Mtb), or a polynucleotide sequence having at least 90% identity or complementarity to a sequence disclosed herein and/or a polynucleotide sequence of a Mtb antigen recognized by T cells.

A lipid nanoparticle (LNP) composition consisting of: a messenger ribonucleic acid (mRNA) encoding one or more Mycobacterium tuberculosis (Mtb) proteins selected from the group consisting of CFP10/Rv3874, ESAT-6/Rv3875, Mtb32A/Rv0125, Mtb39A/Rv1196, Ag85B/Rv1886c, EsxW/Rv3620c, Esx V/Rv3619c, PE13/Rv1195, PPE30/Rv1802, PPE40/Rv2356c and TB10.4/Rv0288; an ionizable cationic lipid comprising a KC3 ionizable cationic lipid at a N/P ratio of 4 to 6 relative to the mRNA, the ionizable cationic lipid present in the LNP composition in a total amount of 46-54 mol % of a total lipid content of the LNP composition; one or more phospholipids selected from the group consisting of distearoylphosphatidylcholine (DSPC), hydrogenated soy phosphatidylcholine (HSPC), and dipalmitoylphosphatidylcholine (DPPC), in a total amount of 10-18 mol % of the total lipid content of the LNP composition; one or more anionic phospholipids selected from the group consisting of dipalmitoylphosphatidyl-L-serine (DPPS), or distearoylphosphatidyl-L-serine (DSPS), distearoylphosphatidylglycerol (DSPG), and dipalmitoyphosphatidylglycerol (DPPG) in a total amount of 2-8 mol % of the total lipid content of the LNP composition; PEG (2000)-dimyristoylglycerol (PEG-DMG) in a total amount of 1-3.5 mol % of the total lipid content of the LNP composition; and cholesterol (e.g., 35.5-40.5 mol % cholesterol).

Aspects of the disclosure relate to a lipid nanoparticle (LNP) composition comprising a KC3 ionizable cationic lipid, cholesterol, one or more phospholipids comprising at least one anionic phospholipid, a conjugated lipid and one or more nucleic acid sequence encoding a T cell epitope from Mycobacterium tuberculosis (Mtb).

In some embodiments, the LNP composition comprises 48 mol % of the KC3 ionizable cationic lipid, 40.5 mol % cholesterol, 5 mol % (L-Serine) DPPS lipid, 5 mol % DSPC or DPPC; and a total of 10 mol % phospholipid concentration, wherein each mol % refers to the mol % of the total lipid content of the LNP composition.

In some embodiments, the LNP composition comprises 48 mol % of the KC3 ionizable cationic lipid, 40.5 mol % cholesterol, and 5 mol % (L-Serine) DPPS lipid, wherein each mol % refers to the mol % of the total lipid content of the LNP composition. In some embodiments, the LNP composition comprises 48 mol % of the KC3 ionizable cationic lipid, 38.5 mol % cholesterol, and 5 mol % (L-Serine) DPPS lipid, wherein each mol % refers to the mol % of the total lipid content of the LNP composition. In some embodiments, the LNP composition comprises 45 mol % of the KC3 ionizable cationic lipid, 42.7 mol % cholesterol, and 5 mol % (L-Serine) DPPS lipid, wherein each mol % refers to the mol % of the total lipid content of the LNP composition. In some embodiments, the LNP composition comprises 50 mol % of the KC3 ionizable cationic lipid, 38.5 mol % cholesterol, 5 mol % (L-Serine) DPPS lipid, and a total of 10 mol % phospholipid concentration, wherein each mol % refers to the mol % of the total lipid content of the LNP composition. In some embodiments, the LNP composition comprises 48 mol % of the KC3 ionizable cationic lipid, 40.5 mol % cholesterol, 5 mol % (L-Serine) DPPS lipid, and a total of 10 mol % phospholipid concentration, wherein each mol % refers to the mol % of the total lipid content of the LNP composition. In some embodiments, the LNP composition comprises 48 mol % of the KC3 ionizable cationic lipid, 40.5 mol % cholesterol, 5 mol % (L-Serine) DPPS lipid, 5 mol % DSPC or DPPC; and a total of 10 mol % phospholipid concentration, wherein each mol % refers to the mol % of the total lipid content of the LNP composition. In some embodiments, the LNP composition comprises 46.5 mol % of the KC3 ionizable cationic lipid, 42 mol % cholesterol, 5 mol % (L-Serine) DPPS lipid, wherein each mol % refers to the mol % of the total lipid content of the LNP composition. In some embodiments, the LNP composition comprises 15 mol % total phospholipid and 35.5 mol % cholesterol. In some embodiments, the LNP composition comprises 10 mol % total phospholipid and 40.5 mol % cholesterol. In some embodiments, the LNP composition comprises 40.5 mol % cholesterol, 5% anionic lipid (DPPS) and 5% PC (DSPC or DPPC) and a total of 10 mol % phospholipid concentration. In some embodiments, the LNP composition comprises 48 mol % cationic ionizable lipid, 5 mol % PC (DPPC), 5 mol % anionic lipid (DPPS), 40.5 mol % cholesterol, 1.5 mol % conjugated lipid (PEG-DMG).

In some aspects, the LNP comprises a nucleic acid sequence (e.g., mRNA) encoding a T cell epitope from Mycobacterium tuberculosis (Mtb), or a Mtb antigen recognized by T cells. In some aspects, the LNP comprises a nucleic acid sequence that is mRNA encoding a concatenated sequence of T-cell epitopes present in Mtb or a Mtb antigen recognized by T Cells. In some aspects, the LNP comprises a nucleic acid sequence that is mRNA encoding one or more Mtb proteins selected from the group consisting of CFP10/Rv3874, ESAT-6/Rv3875, Mtb32A/Rv0125, Mtb39A/Rv1196, and Ag85B/Rv1886c. In some aspects, the LNP comprises a nucleic acid sequence that is mRNA comprising one or more nucleic acid sequences selected from the group consisting of: SEQ ID NO:2, SEQ ID NO:3, SEQ ID NO:6, SEQ ID NO: 7, and SEQ ID NO: 220. In some aspects, the LNP comprises a nucleic acid sequence that is mRNA encoding one or more Mtb proteins selected from the group consisting of EsxW/Rv3620c, EsxV/Rv3619c, PE13/Rv1195, PPE30/Rv1802, PPE40/Rv2356c and TB10.4/Rv0288. In some aspects, the LNP comprises a nucleic acid sequence that is mRNA comprising one or more nucleic acid sequences selected from the group consisting of: SEQ ID NO:1, SEQ ID NO:4, SEQ ID NO:5, SEQ ID NO: 31, SEQ ID NO:221, and SEQ ID NO:222. In some aspects, the LNP comprises a nucleic acid sequence that comprises the concatenated nucleic acid-encoded sequence includes an N-terminal and C-terminal signal peptide selected from Sec/MITD, Lamp1, HLA-Dra, or tPA. In some aspects, the LNP comprises a nucleic acid sequence that is an mRNA having a sequence selected from SEQ ID NOs: 34, 36, 38, 40, 42, 44, 224 and 226. In some aspects, the LNP comprises nucleic acid that is an mRNA encoding an amino acid sequence selected from SEQ ID NOs: 33, 35, 37, 39, 41, 43, 86-105, 207-210, 223 and 225.

In some embodiments, the one or more nucleic acids is a mRNA. In some embodiments, the mRNA encodes a concatenated sequence of T-cell epitopes present in Mtb. In some embodiments, the concatenated sequence of T-cell epitopes comprise an amino acid sequence set forth in SEQ ID NOs: 1-17, 106-137, 138-203. In some embodiments, the concatenated sequence of T-cell epitopes comprises an amino acid sequence with at least 90% sequence identity (e.g. 90%, 91%, 92%, 93%, 94%, 95%, 96%, 97%, 98%, 99% or more) with amino acid sequence set forth in SEQ ID NOs: 1-17, 45-85, 106-137, 138-203. In some embodiments, the concatenated nucleotide sequence comprises two or more sequences encoding for peptides or proteins that can elicit MHC class II-restricted CD4 T cell responses.

In some embodiments, the two or more MHC class II epitopes selected from the group: EsxV (Rv3619), EsxW (Rv3620c), EsxB/CFP10 (Rv3874), EsxA/ESAT-6 (Rv3875), AMtb39A (Rv1196), Ag85B (Rv1886c), and EsxH/TB10.4 (Rv0288). In some embodiments, the two or more MHC class II epitopes comprises peptides or proteins from EsxV (Rv3619), EsxW (Rv3620c), EsxB/CFP10 (Rv3874), EsxA/ESAT-6 (Rv3875), AMtb39A (Rv1196), Ag85B (Rv1886c), and EsxH/TB10.4 (Rv0288) (SEQ ID NOs. 1-7).

In some embodiments, the concatenated nucleic acid-encoded sequence includes the seven proteins in and order N-terminal to C-terminal selected from: EsxB/CFP10 (Rv3874), EsxA/ESAT-6 (Rv3875), EsxH/TB10.4 (Rv0288), AAg85B (Rv1886c), AMtb39A (Rv1196), EsxW (Rv3620c), and EsxV (Rv3619), or EsxB/CFP10 (Rv3874), EsxA/ESAT-6 (Rv3875), EsxW (Rv3620c), EsxV (Rv3619), EsxH/TB10.4 (Rv0288), AAg85B (Rv1886c), and AMtb39A (Rv1196), or EsxB/CFP10 (Rv3874), AMtb39A (Rv1196), EsxA/ESAT-6 (Rv3875), EsxW (Rv3620c), EsxH/TB10.4 (Rv0288), EsxV (Rv3619), and AAg85B (Rv1886c). (SEQ ID NOs. 18, 19, and 20)

In some embodiments, the composition comprises a nucleic acid encoding for 5 or more non-overlapping CD4 T cell epitopes in the form of peptides, wherein optionally the peptides are from 12 to 50 amino acids long.

In some embodiments, the concatenated nucleic acid-encoded sequence optionally comprises 10 selected MHC-II epitopes comprising: AQIYQAVSAQAAAIH (SEQ ID NO. 9), PSPSMGRDIKVQFQS (SEQ ID NO. 10), GINTIPIAINEAEYV (SEQ ID NO. 11), AAFQGAHARFVAAAA (SEQ ID NO. 12), AGWLAFFRDLVARGL (SEQ ID NO. 13), ASIIRLVGAVLAEQH (SEQ ID NO. 14), MSFVTTQPEALAAAA (SEQ ID NO. 8), MHVSFVMAYPEMLAA (SEQ ID NO. 15), AYGSFVRTVSLPVGA (SEQ ID NO. 16), and LENDNQLLYNYPGAL (SEQ ID NO. 17).

In some embodiments, the concatenated nucleic acid-encoded sequence includes GPGPG (SEQ ID NO. 228) linker sequences between each of the concatenated epitopes.

In some embodiments, the one or more nucleic acid comprises a nucleic acid sequence set forth in SEQ ID NOs: 34, 36, 38, 40, 42, and 44.

In some embodiments, the one or more nucleic acid comprises a nucleic acid sequence having at least 90% identity, at least 91%, at least 92%, at least 93%, at least 94%, at least 95%, at least 96%, at least 97%, at least 98% or at least 99% with a nucleic acid sequence set forth in SEQ ID NOs: 34, 36, 38, 40, 42, and 44.

In some embodiments, the concatenated nucleic acid-encoded sequence includes an N-terminal and C-terminal signal peptide selected from Sec/MITD, Lamp1, HLA-Dra, or tPA.

In some embodiments, the one or more nucleic acid comprises a chemically modified mRNA, wherein the chemically modified mRNA comprises N1-methylpseudouridine.

In some embodiments, the one or more nucleic acid comprises a 5′ untranslated region (UTR) and 3′ UTR, polyA tail of about 80 to about 140 nucleotides in length, and (i) a 5′ enzymatic or (ii) a 5′ clean cap.

In some embodiments, the one or more nucleic acid is an mRNA having a sequence selected from SEQ ID NOs: 34, 36, 38, 40, 42, 44, 224 and 226.

In some embodiments, the one or more nucleic acid is an mRNA and wherein the amino acid sequence encoded by the mRNA is selected from SEQ ID NOs: 33, 35, 37, 39, 41, 43, 86-105, 207-210, 223 and 225.

In some embodiments, the nucleic acid-encoded concatenated sequence comprises two or more MHC class I epitopes selected from SEQ ID NOs: 106-137 and 138-203.

In some embodiments, the nucleic acid-encoded concatenated sequence includes two or more MHC class I epitopes found in Mycobacterium tuberculosis, depleted of epitopes found in BCG, and selected from SEQ ID NOs: 86-95.

In some embodiments, the nucleic acid-encoded concatenated sequence includes two or more MHC class I epitopes that are ordered to minimize junctional neoepitope generation, and selected from SEQ ID NOs: 86-105.

In some embodiments, the nucleic acid sequence has at least 90% (e.g. at least 90%, at least 91%, at least 92%, at least 93%, at least 94%, at least 95%, at least 96%, at least 97%, at least 98%, at least 99%) identity to the nucleic acid sequences of the disclosure. In some embodiments, the polypeptide sequence at least 90% identity (e.g. at least 90%, at least 91%, at least 92%, at least 93%, at least 94%, at least 95%, at least 96%, at least 97%, at least 98%, at least 99%) to the polypeptide sequences of the disclosure.

Aspects of the disclosure relate to a method of preventing a bacterial or viral infection, the method comprising administering to a subject in need thereof an effective amount of the composition provided herein to elicit an immune response.

Aspects of the disclosure provide methods of vaccinating a subject comprising administering to the subject a single dosage of the compositions described herein comprising a nucleic acid (e.g. mRNA) encoding a polypeptide in an effective amount to vaccinate the subject. In some embodiments, the nucleic acid is formulated within a cationic lipidic nanoparticle. In some embodiments, the lipidic nanoparticle composition is administered as a single injection.

In some embodiments, the bacterial infection is Mycobacterium tuberculosis infection.

In some embodiments, the lipidic nanoparticle is administered parenterally.

In general, administration to a patient is by intradermal injection is possible. However, injection may also be carried out intranodally into a lymph node (Maloy et al. (2001), Proc Natl Acad Sci USA 98:3299-3033). The resulting cells present the complex of interest and are recognized by autologous cytotoxic T lymphocytes which then propagate.

In some embodiments, the composition is administered by inhalation. In some embodiments, the composition is formulated as nasal spray, and/or aerosol.

Actual dosage levels of the active agents in the pharmaceutical compositions disclosed herein may be varied so as to obtain an amount of the active agent which is effective to achieve the desired therapeutic response for a particular patient, composition, and mode of administration, without being toxic to the patient.

“Parenteral” as used herein in the context of administration means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, inhalation, subcapsular, subarachnoid, respiratory mucosal, intraspinal, epidural and intrasternal injection and infusion. Intravenous injection and infusion are often (but not exclusively) used for liposomal drug administration.

Dosage regimens can be adjusted to provide the optimum desired response (e.g., a therapeutic response). For example, one or more doses may be administered over time or the dose may be proportionally reduced or increased as indicated by the exigencies of the therapeutic situation.

In some embodiments, the dose comprises between 0.01 to 5 mg/kg of nucleic acid. In some embodiments, the dose comprises between 0.01 to 5 mg/kg of mRNA. In some embodiments, the dose comprises between 0.01 to 3 mg/kg of nucleic acid. In some embodiments, the dose comprises between 0.01 to 3 mg/kg of mRNA. In some embodiments, the dose comprises between 0.01 to 1 mg/kg of nucleic acid. In some embodiments, the dose comprises between 0.01 to 1 mg/kg of mRNA. In some embodiments, the dose comprises between 0.01 to 0.5 mg/kg of nucleic acid. In some embodiments, the dose comprises between 0.01 to 0.5 mg/kg of mRNA. In some embodiments, the dose comprises between 0.01 to 1 mg/kg of mRNA. In some embodiments, the dose comprises between 0.01 to 0.1 mg/kg of nucleic acid. In some embodiments, the dose comprises between 0.01 to 0.05 mg/kg of mRNA. In some embodiments, the dose comprises between 0.01 to 0.1 mg/kg of nucleic acid. In some embodiments, the dose comprises between 0.01 to 0.05 mg/kg of mRNA.

The dosage of the compounds and/or of their pharmaceutically acceptable salts or the LNPs comprising the compounds and/or of their pharmaceutically acceptable salts may vary within wide limits and should naturally be adjusted, in each particular case, to the individual conditions and to the pathogenic agent to be controlled.

### Additional Embodiments

The following additional embodiments are provided for illustrative purposes.

Embodiment 1: A lipid nanoparticle (LNP) composition comprising a KC3 ionizable cationic lipid, cholesterol, one or more phospholipids comprising at least one anionic phospholipid, a conjugated lipid and one or more nucleic acid sequence encoding a T cell epitope from Mycobacterium tuberculosis (Mtb).

Embodiment 2: The composition of embodiment 1, wherein the composition comprises:
48 mol % of the KC3 ionizable cationic lipid,
40.5 mol % cholesterol,
5 mol % (L-Serine) DPPS lipid,
5 mol % DSPC or DPPC; and
a total of 10 mol % phospholipid concentration;
wherein each mol % refers to the mol % of the total lipid content of the LNP composition.

Embodiment 3: The composition of embodiment 1 or embodiment 2, wherein the one or more nucleic acids is a mRNA.

Embodiment 4: The composition of embodiment 3, wherein the mRNA encodes a concatenated sequence of T-cell epitopes present in Mtb.

Embodiment 5: The composition of embodiment 4, wherein the concatenated sequence of T-cell epitopes comprise an amino acid sequence set forth in SEQ ID NOs: 1-17, 106-137, 138-203.

Embodiment 6: The composition of embodiment 4, wherein the concatenated sequence of T-cell epitopes comprises an amino acid sequence with at least 90% sequence identity with amino acid sequence set forth in SEQ ID NOs: 1-17, 45-85, 106-137, 138-203.

Embodiment 7: The composition of embodiment 5 or embodiment 6, wherein the concatenated nucleotide sequence comprises two or more sequences encoding for peptides or proteins that can elicit MHC class II-restricted CD4 T cell responses.

Embodiment 8: The composition of embodiment 7, wherein the two or more MHC class II epitopes selected from the group: EsxV (Rv3619), EsxW (Rv3620c), EsxB/CFP10 (Rv3874), EsxA/ESAT-6 (Rv3875), ΔMtb39A (Rv1196), Ag85B (Rv1886c), and EsxH/TB10.4 (Rv0288).

Embodiment 9: The composition of embodiment 7, wherein the two or more MHC class II epitopes comprises peptides or proteins from EsxV (Rv3619), EsxW (Rv3620c), EsxB/CFP10 (Rv3874), EsxA/ESAT-6 (Rv3875), ΔMtb39A (Rv1196), Ag85B (Rv1886c), and EsxH/TB10.4 (Rv0288) (SEQ ID NOs. 1-7).

Embodiment 10: The composition of embodiment 5 or embodiment 6 wherein the concatenated nucleic acid-encoded sequence includes the seven proteins in and order N-terminal to C-terminal selected from:
EsxB/CFP10 (Rv3874), EsxA/ESAT-6 (Rv3875), EsxH/TB10.4 (Rv0288), ΔAg85B (Rv1886c), ΔMtb39A (Rv1196), EsxW (Rv3620c), and Esx V (Rv3619), or
EsxB/CFP10 (Rv3874), EsxA/ESAT-6 (Rv3875), EsxW (Rv3620c), EsxV (Rv3619), EsxH/TB10.4 (Rv0288), ΔAg85B (Rv1886c), and ΔMtb39A (Rv1196), or
EsxB/CFP10 (Rv3874), ΔMtb39A (Rv1196), EsxA/ESAT-6 (Rv3875), EsxW (Rv3620c), EsxH/TB10.4 (Rv0288), EsxV (Rv3619), and ΔAg85B (Rv1886c). (SEQ ID NOs. 18, 19, and 20)

Embodiment 11: The composition of any one of embodiments 7-10, the composition comprising a nucleic acid encoding for 5 or more non-overlapping CD4 T cell epitopes in the form of peptides, wherein optionally the peptides are from 12 to 50 amino acids long.

Embodiment 12: The composition of embodiment 5 or embodiment 6, wherein the concatenated nucleic acid-encoded sequence optionally comprises 10 selected MHC-II epitopes comprising: AQIYQAVSAQAAAIH (SEQ ID NO. 9), PSPSMGRDIKVQFQS (SEQ ID NO. 10), GINTIPIAINEAEYV (SEQ ID NO. 11), AAFQGAHARFVAAAA (SEQ ID NO. 12), AGWLAFFRDLVARGL (SEQ ID NO. 13), ASIIRLVGAVLAEQH (SEQ ID NO. 14), MSFVTTQPEALAAAA (SEQ ID NO. 8), MHVSFVMAYPEMLAA (SEQ ID NO. 15), AYGSFVRTVSLPVGA (SEQ ID NO. 16), and LENDNQLLYNYPGAL (SEQ ID NO. 17).

Embodiment 13: The composition of embodiment 7, wherein the concatenated nucleic acid-encoded sequence includes GPGPG (SEQ ID NO: 228) linker sequences between each of the concatenated epitopes.

Embodiment 14: The composition of any one of embodiments 3-13, wherein the one or more nucleic acid comprises a nucleic acid sequence set forth in SEQ ID NOs: 34, 36, 38, 40, 42, and 44.

Embodiment 15: The composition of any one of embodiments 3-13, wherein the one or more nucleic acid comprises a nucleic acid sequence having at least 90% identity, at least 95%, or at least 99% with a nucleic acid sequence set forth in SEQ ID NOs: 34, 36, 38, 40, 42, and 44.

Embodiment 16: The composition of any one of embodiments 3-13, wherein the concatenated nucleic acid-encoded sequence includes an N-terminal and C-terminal signal peptide selected from Sec/MITD, Lamp1, HLA-Dra, or tPA.

Embodiment 17: The composition of any one of embodiments 3-16, wherein the one or more nucleic acid comprises a chemically modified mRNA, wherein the chemically modified mRNA comprises N1-methylpseudouridine.

Embodiment 18: The composition of any one of embodiments 3-16, wherein the one or more nucleic acid comprises a 5′ untranslated region (UTR) and 3′ UTR, polyA tail of about 80 to about 140 nucleotides in length, and (i) a 5′ enzymatic or (ii) a 5′ clean cap.

Embodiment 19: The composition of any one of embodiments 3-16, wherein the one or more nucleic acid is an mRNA having a sequence selected from SEQ ID NOs: 34, 36, 38, 40, 42, and 44.

Embodiment 20: The composition of any one of embodiments 3-16, wherein the one or more nucleic acid is an mRNA and wherein the amino acid sequence encoded by the mRNA is selected from SEQ ID NOs: 33, 35, 37, 39, 41, 43, 86-105, and 207-210.

Embodiment 21: The composition of embodiment 5 or embodiment 6, wherein the nucleic acid-encoded concatenated sequence comprises two or more MHC class I epitopes selected from SEQ ID NOs: 106-137 and 138-203.

Embodiment 22: The composition of embodiment 5 or embodiment 6, wherein the nucleic acid-encoded concatenated sequence includes two or more MHC class I epitopes found in Mycobacterium tuberculosis, depleted of epitopes found in BCG, and selected from SEQ ID NOs: 86-95.

Embodiment 23: The composition of embodiment 5 or embodiment 6, wherein the nucleic acid-encoded concatenated sequence includes two or more MHC class I epitopes that are ordered to minimize junctional neoepitope generation, and selected from SEQ ID NOs: 86-105.

Embodiment 24: The composition of any one of embodiments 1-23, wherein the cationic lipid is KC3-OA, KC3-PA, KC3-01, KC3-C17 (8:1), or KC3-C15 (C8:1).

Embodiment 25: The composition of any one of embodiments 1-24, wherein the LNP comprises the conjugated lipid in a total amount of less than 2 mol % of the total lipid content of the LNP composition.

Embodiment 26: The composition of any one of embodiments 1-24, wherein the ionizable cationic lipid in a total amount of 45-55 mol % of the total lipid content of the LNP composition;
wherein cholesterol is in a total amount of 35-45 mol % of the total lipid content of the LNP composition;
wherein the total amount of the one more phospholipid is 7-15 mol % of the total lipid content of the LNP composition;
wherein the one or more phospholipids consist of DSPC and the PS lipid is one or more lipids selected from the group consisting of the L-serine configuration of DPPS and DSPS; and
the total amount of the PS lipid is about 5 mol % of the total lipid content of the LNP composition.

Embodiment 27: The composition of any one of embodiments 1-24,
wherein the conjugated lipid is PEG-DMG; and
wherein the PS lipid is selected from the group consisting of: DSPS (L-isomer) and DPPS.

Embodiment 28: The composition of any one of embodiments 1-27, wherein the ionizable cationic lipid is KC3-OA.

Embodiment 29: The composition of any one of embodiments 1-28, wherein the LNP composition has a N/P ratio of 4 to 7.

Embodiment 30: The composition of any one of embodiments 1-28, wherein the LNP composition has a N/P ratio of 5 to 6.

Embodiment 31: A nucleic acid lipid nanoparticle (LNP) composition comprising: a mRNA having at least 90% identity with a nucleic acid sequence set forth in SEQ ID NOs: 34, 36, 38, 40, 42, and 44, ionizable cationic lipid KC3-PA, and a (L-Serine) PS lipid in a total amount of 2.5-10 mol % of the total lipid content of the LNP composition.

Embodiment 32: The composition of embodiment 31, wherein the PS lipid is (L-Serine) DSPS, (L-Serine) DPPS, or a mixture thereof, and the LNP composition further comprises cholesterol and a second phospholipid selected from the group consisting of: DSPC, DOPC, DPPC, HSPC, and SM.

Embodiment 33: A nucleic acid lipid nanoparticle (LNP) composition comprising:
a mRNA having at least 90% identity with a nucleic acid sequence set forth in SEQ ID NOs: 34, 36, 38, 40, 42, and 44;
a KC3 ionizable cationic lipid in a total amount of 40-65 mol % of the total lipid content of the LNP composition;
cholesterol in a total amount of 23.5-43.5 mol % of the total lipid content of the LNP composition;
a (L-Serine) PS lipid in a total amount of 2.5-10 mol % of the total lipid content of the LNP composition;
DSPC or HSPC phospholipid in a total amount of 5-25 mol % of the total lipid content of the LNP composition; and
a PEG-containing conjugated lipid in a total amount of 0.5 mol % to 2.5 mol % of the total lipid content of the LNP composition.

Embodiment 34: The composition of embodiment 1 comprising an ionizable lipid having the chemical structure:

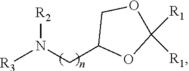

wherein
R₁ is

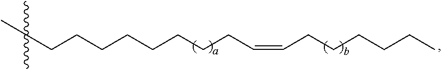

wherein a is 0 or 1; b is 1, 2, 3 or 4, provided the sum a+b is 1, 2, 3 or 4;
R₂ and R₃ are each independently methyl; and
n is an integer equal to 2 or 3.

Embodiment 35: The composition of embodiment 34, wherein n is 3.

Embodiment 36: The composition of any of the preceding embodiments, wherein the composition is a vaccine.

Embodiment 37: A pharmaceutical composition comprising the lipid nanoparticle of any one of the precenting embodiments, and a pharmaceutically acceptable carrier.

Embodiment 38: A nucleic acid encoding a concatenated amino acid sequence of T-cell epitopes present in Mycobacterium tuberculosis, the nucleic acid having at least 90% identity with a nucleic acid sequence set forth in SEQ ID NOs: 34, 36, 38, 40, 42, and 44.

### EXAMPLES

While this disclosure has been described in relation to certain embodiments, and many details have been set forth for purposes of illustration, it will be apparent to those skilled in the art that this disclosure includes additional embodiments, and that some of the details described herein may be varied considerably without departing from this disclosure. This disclosure includes such additional embodiments, modifications and equivalents. In particular, this disclosure includes any combination of the features, terms, or elements of the various illustrative components and examples.

Unless explicitly indicated otherwise, the isomer form of the phosphatidylserine lipids used in the Examples is phosphatidyl-L-serine.

Certain examples are provided below to illustrate various embodiments of the embodiments disclosed herein. One of ordinary skill in the art will recognize that the various embodiments disclosed herein are not limited to these specific illustrative examples.

### Example 1. Synthesis of Ionizable Lipids

1. 2-((S)-2,2-di((6Z,12Z)-octadeca-6,12-dien-1-yl)-1,3-dioxolan-4-yl)-N,N-dimethylethan-1-amine (AKG-KC2-01, O-12095)
2. 3-((S)-2,2-di((6Z,12Z)-octadeca-6,12-dien-1-yl)-1,3-dioxolan-4-yl)-N,N-dimethylpropan-1-amine (AKG-KC3-01, O-12096)
3. 2-((S)-2,2-di((Z)-octadec-9-en-1-yl)-1,3-dioxolan-4-yl)-N,N-dimethylethan-1-amine (AKG-KC2-OA, O-11880)
4. 2-((S)-2,2-di((Z)-hexadec-9-en-1-yl)-1,3-dioxolan-4-yl)-N,N-dimethylethan-1-amine (AKG-KC2-PA, O-11879)
5. 3-((S)-2,2-di((Z)-octadec-9-en-1-yl)-1,3-dioxolan-4-yl)-N,N-dimethylpropan-1-amine (AKG-KC3-OA, O-11957)
6. 3-((S)-2,2-di((Z)-hexadec-9-en-1-yl)-1,3-dioxolan-4-yl)-N,N-dimethylpropan-1-amine (AKG-KC3-PA, O-12418)
7. 3-((S)-2,2-di((Z)-heptadec-8-en-1-yl)-1,3-dioxolan-4-yl)-N,N-dimethylpropan-1-amine (AKG-KC3-C17 (C8:1))
8. (S)-3-(2,2-diheptadecyl-1,3-dioxolan-4-yl)-N,N-dimethylpropan-1-amine (AKG-KC3-C17)

### Synthesis of 2-((S)-2,2-di((6Z,12Z)-octadeca-6,12-dien-1-yl)-1,3-dioxolan-4-yl)-N,N-dimethylethan-1-amine (AKG-KC2-01, O-12095)

### 3-((S)-2,2-di((6Z,12Z)-octadeca-6,12-dien-1-yl)-1,3-dioxolan-4-yl)-N,N-dimethylpropan-1-amine (AKG-KC3-01, O-12096)

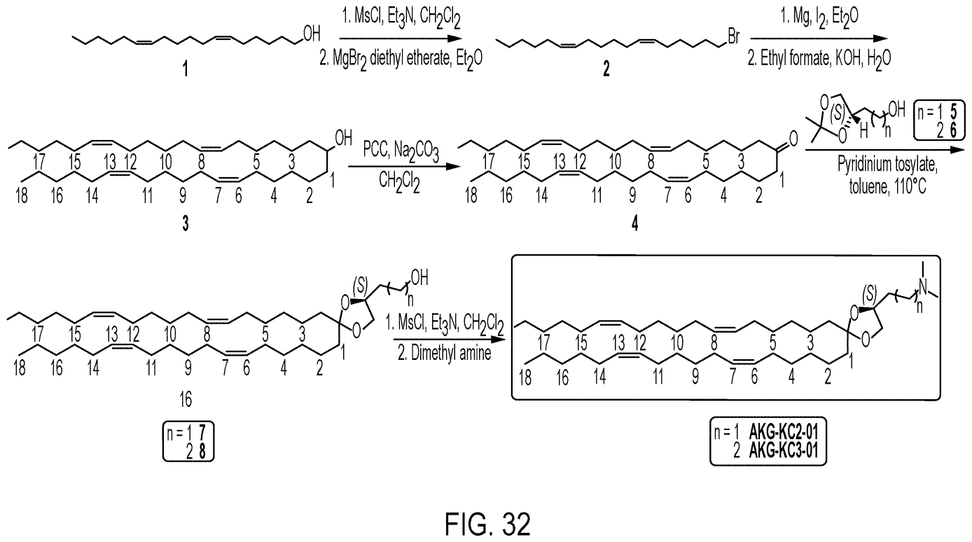

FIG. 32 is a scheme showing the synthesis of AKG-KC2-01 and AKG-KC3-01.

### Experimental Procedure

### Synthesis of (6Z,12Z)-1-bromooctadeca-6,12-diene, 2

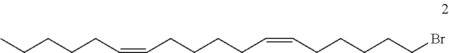 [SMILES: CCCCC/C=C\CCCC/C=C\CCCCCBr]

To a solution of (6Z,12Z)-octadeca-6,12-dien-1-ol, 1 (3.6 g, 13.7 mmol) in dichloromethane (50 mL) at 0° C. was added methane sulfonyl chloride (1.26 mL, 16.4 mmol) and triethylamine (3.6 mL, 20.5 mmol). The resulting solution was warmed to room temperature and stirred for 2 hours. The mixture was quenched with water and extracted with dichloromethane (2×100 mL). The combined organics were washed with brine then dried over magnesium sulfate then filtered. The filtrate was concentrated under vacuum to give a crude oil. The resulting oil was dissolved in diethyl ether (50 mL), added to a stirring slurry of magnesium bromide ethyl etherate (7 g, 27.4 mmol) in diethyl ether (50 mL) at OC. The mixture was warmed to room temperature and stirred for 2 hours. The reaction mixture was quenched with water and extracted with ethyl acetate (2×100 mL). The combined organics were washed with brine then dried over magnesium sulfate then filtered. The filtrate was concentrated under vacuum to give a crude oil. The crude oil was purified by chromatography on silica using 5-10% ethyl acetate in n-hexane as eluant to give (6Z,12Z)-1-bromooctadeca-6,12-diene, 3 (2.9 g, 8.89 mmol, 65%) as a yellow oil.

¹H NMR (300 MHz, CDCl₃): 5.36-5.33 (m, 4H), 3.42-3.37 (t, J=7.5 Hz, 2H), 2.04-1.97 (m, 8H), 1.83-1.83 (m, 2H), 1.37-1.28 (m, 14H), 0.90-0.86 (t, J=6.6 Hz, 3H).

### Synthesis of (6Z,12Z,25Z,31Z)-heptatriaconta-6,12,25,31-tetraen-19-ol, 3

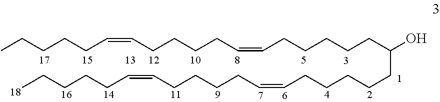 [SMILES: CCCCC/C=C\CCCC/C=C\CCCCCC(O)CCCCC/C=C\CCCC/C=C\CCCCC]

A solution of (6Z,12Z)-1-bromooctadeca-6,12-diene, 2 (2 g, 6.08 mmol) in ether (10 mL) was added to a mixture of magnesium turnings (162 mg, 6.69 mmol) and iodine in ether (2 mL) under argon at room temperature. The mixture stirred at room temperature for 90 minutes (magnesium turnings consumed) whereupon ethyl formate (0.24 mL, 3.04 mmol) was added. After stirring for one hour at room temperature, the reaction was quenched with 1N HCl solution. The mixture was extracted with ethyl acetate (2×100 mL) and the combined organics washed with water then brine. The organics were dried under magnesium sulfate, filtered, and the filtrate concentrated under vacuum to give a crude oil. The resulting oil was dissolved in ethanol (10 mL) and added to a solution of potassium hydroxide (260 mg) in water (3 mL). After stirring for 12 hours, the mixture pH was adjusted 4 with 2N HCl. The aqueous solution was extracted with dichloromethane (2×) and combined. The organics were washed with brine then dried under magnesium sulfate and filtered. The filtrate was concentrated under vacuum to give a crude oil. Purification of the crude oil on silica using 10-30% ethyl acetate in n-hexane as eluant to give (6Z,12Z,25Z,31Z)-heptatriaconta-6,12,25,31-tetraen-19-ol, 3 (0.29 g, 0.55 mmol, 18%) as a clear oil.

¹H NMR (300 MHz, CDCl₃): 5.36-5.32 (m, 8H), 3.57 (bs, 1H), 3.33-3.32, (m, 2H), 2.13-1.97 (m, 16H), 1.36-1.29 (m, 34H), 0.90-0.86 (t, J=6.6 Hz, 6H).

### Synthesis of (6Z,12Z,25Z,31Z)-heptatriaconta-6,12,25,31-tetraen-19-one, 4

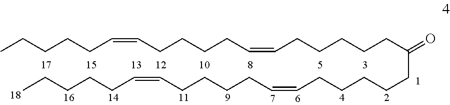 [SMILES: CCCCC/C=C\CCCC/C=C\CCCCCC(=O)CCCCC/C=C\CCCC/C=C\CCCCC]

To a mixture of (6Z,12Z,25Z,31Z)-heptatriaconta-6,12,25,31-tetraen-19-ol, 3 (0.29 g, 0.55 mmol) and sodium carbonate (3 mg, 0.03 mmol) in dichloromethane was added pyridinium chlorochromate (236 mg, 1.1 mmol) at 0° C. The mixture was warmed to room temperature and stirred for one hour. After one hour, silica gel (1 g) was added to reaction and the mixture filtered. The filtrate was concentrated, and the resulted oil purified on silica using 10-20% ethyl acetate in n-hexane as eluant to give (6Z,12Z,25Z,31Z)-heptatriaconta-6,12,25,31-tetraen-19-one, 4 (0.12 g, 0.23 mmol, 42%) as a clear oil.

¹H NMR (300 MHz, CDCl₃): 5.36-5.32 (m, 8H), 3.36-3.32, (m, 1H), 2.40-2.35 (t, J=6.6 Hz, 3H), 2.14-2.00 (m, 16H), 1.58-1.54 (m, 4H), 1.34-1.29 (m, 28H), 0.90-0.86 (t, J=6.6 Hz, 6H).

### Synthesis of 2-((S)-2,2-di((9Z,12Z)-octadeca-9,12-dien-1-yl)-1,3-dioxolan-4-yl) ethan-1-ol, 7

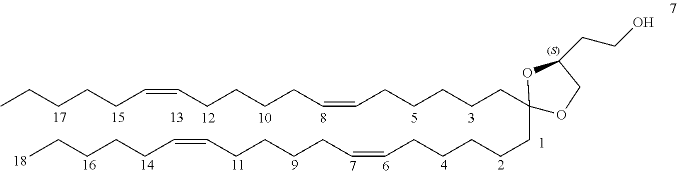

A mixture of (6Z,12Z,25Z,31Z)-heptatriaconta-6,12,25,31-tetraen-19-one, 4 (0.12 g, 0.23 mmol), (4S)-(+)-4-(2-hydroxyethyl)-2,2-dimethyl-1,3-dioxolane 5 (0.20 g, 1.38 mmol), and pyridinium p-toluene sulfonate (9 mg) in toluene (10 mL) was heated at reflux under nitrogen positive pressure. After 12 hours, the mixture was concentrated under vacuum to give a crude oil. The resulting crude oil was purified by chromatography on silica using 20-40% ethyl acetate in n-hexane as eluant to give 2-((S)-2,2-di((9Z,12Z)-octadeca-9,12-dien-1-yl)-1,3-dioxolan-4-yl) ethan-1-ol, 7 (0.11 g, 0.17 mmol, 77%) as a clear oil.

¹H NMR (300 MHz, CDCl₃): 5.36-5.32 (m, 8H), 4.25-4.20 (m, 1H), 4.10-4.06 (m, 1H), 3.82-3.77 (m, 1H), 3.54-3.49 (m, 1H), 2.23-2.19 (t, J=6.6 Hz, 3H), 2.14-2.00 (m, 16H), 1.84-1.78 (m, 2H), 1.62-1.51 (m, 6H), 1.34-1.29 (m, 28H), 0.90-0.86 (t, J=6.6 Hz, 6H).

### Synthesis of 3-((S)-2,2-di((9Z,12Z)-octadeca-9,12-dien-1-yl)-1,3-dioxolan-4-yl) propan-1-ol, 8

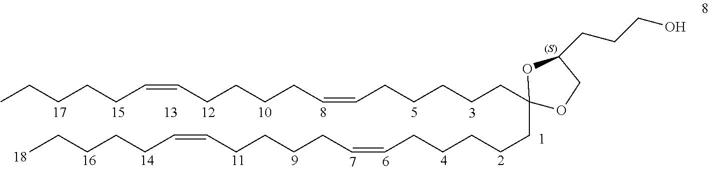

A mixture of (6Z,12Z,25Z,31Z)-heptatriaconta-6,12,25,31-tetraen-19-one, 4 (0.50 g, 0.95 mmol), (S)-(3)-(2,2-Dimethyl-1,3-dioxolane-4-yl) propanol 6 (0.76 g, 4.75 mmol), and pyridinium p-toluene sulfonate (36 mg) in toluene (10 mL) was heated at reflux under nitrogen positive pressure. After 12 hours, the mixture was concentrated under vacuum to give a crude oil. The resulting crude oil was purified by chromatography on silica using 20-40% ethyl acetate in n-hexane as eluant to 3-((S)-2,2-di((9Z,12Z)-octadeca-9,12-dien-1-yl)-1,3-dioxolan-4-yl) propan-1-ol, 8 (0.48 g, 0.76 mmol, 80%) as a clear oil.

¹H NMR (300 MHz, CDCl₃): 5.34-5.29 (m, 8H), 4.06-4.02 (m, 2H), 3.67-3.47 (m, 2H), 3.45-3.43 (m, 1H), 2.12-2.01 (m, 16H), 1.65-1.62 (m, 8H), 1.34-1.29 (m, 32H), 0.89-0.85 (t, J=6.6 Hz, 6H).

### Synthesis of 2-((S)-2,2-di((9Z,12Z)-octadeca-9,12-dien-1-yl)-1,3-dioxolan-4-yl)-N,N-dimethylethan-1-amine, (AKG-KC2-01, O-12095)

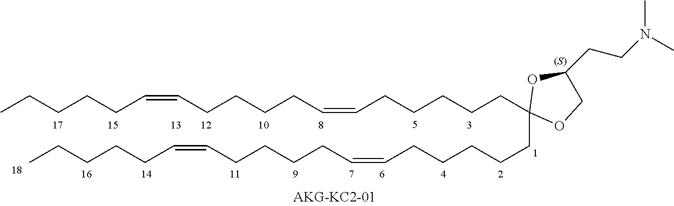

To a solution of 2-((S)-2,2-di((9Z,12Z)-octadeca-9,12-dien-1-yl)-1,3-dioxolan-4-yl) ethan-1-ol, 7 (0.49 g, 0.79 mmol) in dichloromethane (10 mL) at 0° C. was added methanesulfonyl chloride (73 μL, 0.95 mmol) and triethylamine (0.26 mL, 1.2 mmol). The solution was warmed to room temperature and stirred for an addition hour. The reaction was quenched with water and extracted with dichloromethane (2×100 mL). The organics were washed with brine then dried over magnesium sulfate and filtered. The filtrate was concentrated under vacuum to give a crude oil. A solution of 2M dimethylamine (10 mL) was added to the resulting crude oil and allowed to stir for 24 hours. The mixture was then quenched with water and extracted with dichloromethane (2×100 mL). The combined organics were washed with brine then dried over magnesium sulfate then filtered. The filtrate was concentrated under vacuum to give a crude oil. The crude oil was purified by chromatography on silica using 5-100% ethyl acetate in n-hexane as eluant to give 2-((S)-2,2-di((9Z,12Z)-octadeca-9,12-dien-1-yl)-1,3-dioxolan-4-yl)-N,N-dimethylethan-1-amine, (AKG-KC2-01, O-12095), (206 mg, 0.32 mmol, 41%) as a clear oil.

¹H NMR (300 MHz, CDCl₃): 5.35-5.32 (m, 8H), 4.08-4.03 (m, 2H), 3.47 (t, J=6.8 Hz, 1H), 2.36-2.27 (m, 2H), 2.21 (s, 6H), 2.01-1.99 (m, 16H), 1.88-1.77 (m, 2H), 1.68-1.53 (m, 6H), 1.42-1.19 (m, 34H), 0.96-0.86 (t, J=3.7 Hz, 6H).

MS(APCI) for C₄₃H₇₉NO₂: 642.6

### Synthesis of 3-((S)-2,2-di((6Z, 12Z)-octadeca-6-12-dien-4-yl)-1,3-dioxolan-4-yl)-N,N-dimethylpropan-1-amine, AKG-KC3-01, O-12096)

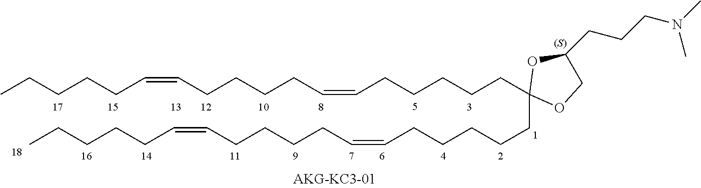

The procedure was previously described.

3-((S)-2,2-di((6Z, 12Z)-octadeca-6-12-dien-4-yl)-1,3-dioxolan-4-yl)-N,N-dimethylpropan-1-amine, (AKG-KC3-01, O-12096), (255 mg, 0.39 mmol, 51%) as a clear oil.

¹H NMR (300 MHz, CDCl₃): 5.39-5.32 (m, 8H), 4.06-4.02 (m, 2H), 3.48-3.44 (m, 1H), 2.35-2.30 (m, 2H), 2.25 (s, 6H), 2.01-1.98 (m, 16H), 1.70-1.51 (m, 12H), 1.35-1.25 (m, 32H), 0.90-0.85 (t, J=6.6 Hz, 6H).

MS(APCI) for C₄₄H₈₁NO₂: 656.6

### Synthesis of 2-((S)-2,2-di((Z)-octadec-9-en-1-yl)-1,3-dioxolan-4-yl)-N,N-dimethylethan-1-amine (AKG-KC2-OA, O-11880)

### 2-((S)-2,2-di((Z)-hexadec-9-en-1-yl)-1,3-dioxolan-4-yl)-N,N-dimethylethan-1-amine (AKG-KC2-PA, O-11879)

### 3-((S)-2,2-di((Z)-octadec-9-en-1-yl)-1,3-dioxolan-4-yl)-N,N-dimethylpropan-1-amine (AKG-KC3-OA, O-11957)

### 3-((S)-2,2-di((Z)-hexadec-9-en-1-yl)-1,3-dioxolan-4-yl)-N,N-dimethylpropan-1-amine, (AKG-KC3-PA, O-12418

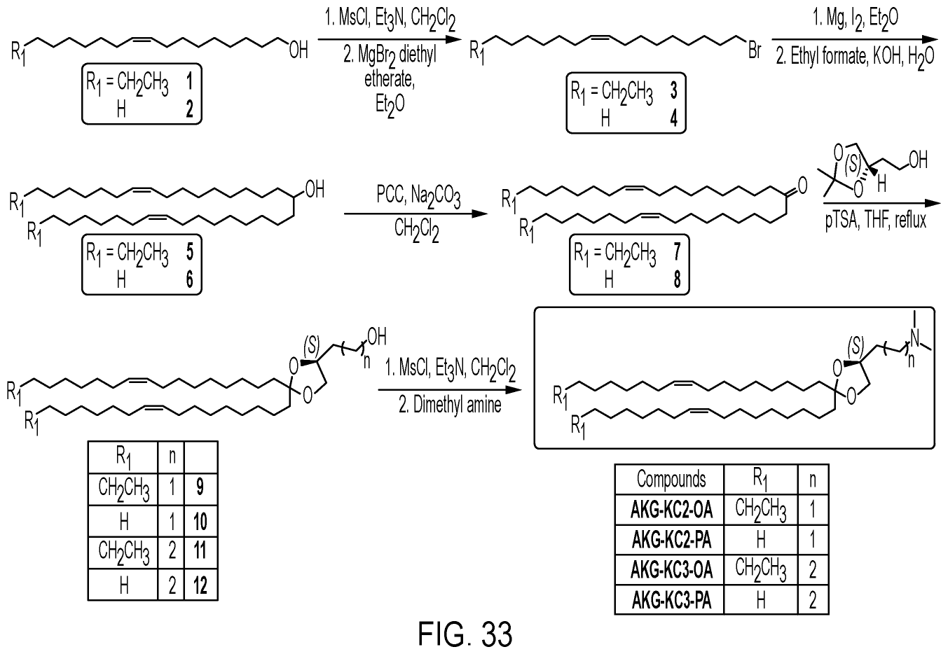

FIG. 33 is a scheme showing the synthesis of AKG-KC2-OA, AKG-KC2-PA, AKG-KC3-OA, and AKG-KC3-PA

### Experimental Procedure (Refer to Previously Described Synthesis of AKG-KC2-01)

### Synthesis of (Z)-1-bromooctadec-9-ene 3

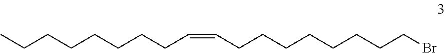 [SMILES: CCCCCCCC/C=C\CCCCCCCCBr]

The procedure was previously described.

(Z)-1-bromooctadec-9-ene, (6.4 g, 19.33 mmol) as a clear oil.

¹H NMR (300 MHz, CDCl₃): 5.36-5.32 (m, 2H), 3.41 (t, J=7.5 Hz, 2H), 2.01-1.99 (m, 4H), 1.87-1.82 (m, 2H), 1.44-1.26 (m, 22H), 0.87 (t, J=6.6 Hz, 3H).

### (Z)-16-bromohexadec-7-ene 4

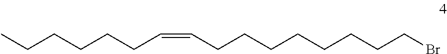 [SMILES: CCCCCC/C=C\CCCCCCCCBr]

¹H NMR (300 MHz, CDCl₃): 5.36-5.32 (m, 2H), 3.42 (t, J=7.5 Hz, 2H), 2.01-1.99 (m, 4H), 1.87-1.82 (m, 2H), 1.44-1.26 (m, 18H), 0.89 (t, J=6.6 Hz, 3H).

### Synthesis of (9Z,28Z)-heptatriaconta-9,28-dien-19-ol 5

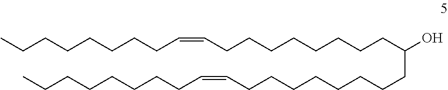 [SMILES: CCCCCCCC/C=C\CCCCCCCCC(O)CCCCCCCC/C=C\CCCCCCCC]

The procedure was previously described.

(9Z,28Z)-heptatriaconta-9,28-dien-19-ol (1.2 g, 2.25 mmol, 47%) as a solid.

¹H NMR (300 MHz, CDCl₃): 5.36-5.29 (m, 4H), 3.57 (bs, 1H), 2.01-1.97 (m, 8H), 1.42-1.26 (m, 53H), 0.89 (t, J=6.6 Hz, 6H).

### (7Z,26Z)-tritriaconta-7.26-dien-17-ol 6

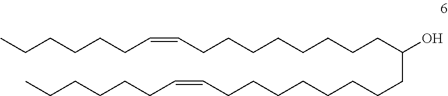 [SMILES: CCCCCC/C=C\CCCCCCCCC(O)CCCCCCCC/C=C\CCCCCC]

¹H NMR (300 MHz, CDCl₃): 5.36-5.29 (m, 4H), 3.57 (bs, 1H), 2.01-1.97 (m, 8H), 1.42-1.26 (m, 45H), 0.89 (t, J=6.6 Hz, 6H).

### Synthesis of (9Z,28Z)-heptatriaconta-9,28-dien-19-one 7

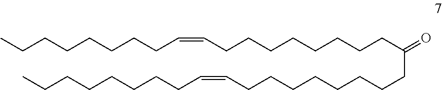 [SMILES: CCCCCCCC/C=C\CCCCCCCCC(=O)CCCCCCCC/C=C\CCCCCCCC]

The procedure was previously described.

(9Z,28Z)-heptatriaconta-9,28-dien-19-one (0.89 g, 1.67 mmol, 74%) as a clear oil.

¹H NMR (300 MHz, CDCl₃): 5.36-5.29 (m, 4H), 2.03-1.98 (m, 8H), 1.42-1.26 (m, 52H), 0.90-0.89 (t, J=6.6 Hz, 6H).

### (7Z,26Z)-tritriaconta-7,26-dien-17-one 8

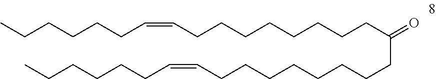 [SMILES: CCCCCC/C=C\CCCCCCCCC(=O)CCCCCCCC/C=C\CCCCCC]

¹H NMR (300 MHz, CDCl₃): 5.36-5.29 (m, 4H), 2.03-1.98 (m, 8H), 1.42-1.26 (m, 44H), 0.90-0.89 (t, J=6.6 Hz, 6H).

### Synthesis of 2-((S)-2,2-di((Z)-octadec-9-en-1-yl)-1,3-dioxolan-4-yl) ethan-1-ol 9

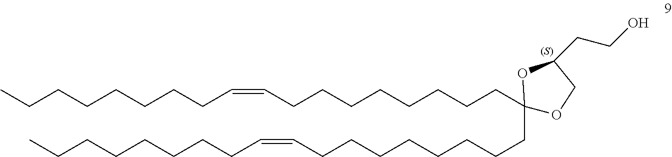

The procedure was previously described.

2-((S)-2,2-di((Z)-octadec-9-en-1-yl)-1,3-dioxolan-4-yl) ethan-1-ol (0.39 g, 0.63 mmol, 74%) as a clear oil.

¹H NMR (300 MHz, CDCl₃): 5.36-5.28 (m, 4H), 4.22-4.10 (m, 1H), 4.08-4.05 (m, 1H), 3.82-3.79 (m, 2H), 3.48 (t, J=6.8 Hz, 1H), 2.24-2.21 (m, 1H), 2.01-1.99 (m, 8H), 1.81-1.80 (m, 2H), 1.59-1.54 (m, 6H), 1.34-1.26 (m, 45H), 0.87 (t, J=6.3 Hz, 6H).

### Synthesis of 2-((S)-2,2-di((Z)-hexadec-9-en-1-yl)-1,3-dioxolan-4-yl) ethan-1-ol, 10

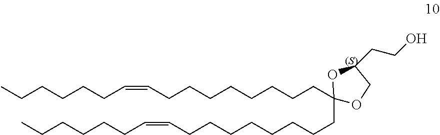

The procedure was previously described.

2-((S)-2,2-di((Z)-hexadec-9-en-1-yl)-1,3-dioxolan-4-yl) ethan-1-ol (1.02 g, 1.65 mmol, 51%) as a clear oil.

¹H NMR (300 MHz, CDCl₃): 5.36-5.29 (m, 4H), 4.23-4.10 (m, 1H), 4.07-4.05 (m, 1H), 3.82-3.79 (m, 2H), 3.48 (t, J=6.6 Hz, 1H), 2.24-2.12 (m, 1H), 2.01-1.97 (m, 8H), 1.84-1.78 (m, 2H), 1.57-1.55 (m, 8H), 1.34-1.29 (m, 35H), 0.87 (t, J=6.3 Hz, 6H).

### Synthesis of 3-((S)-2,2-di((Z)-octadec-9-en-1-yl)-1,3-dioxolan-4-yl) propan-1-ol, 11

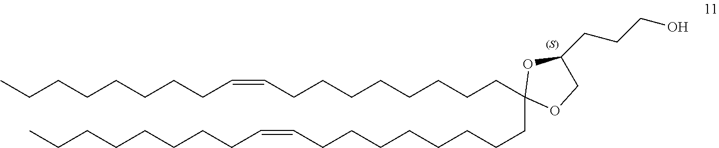

The procedure was previously described.

3-((S)-2,2-di((Z)-octadec-9-en-1-yl)-1,3-dioxolan-4-yl) propan-1-ol (0.41 g, 0.65 mmol, 76%) as a clear oil

¹H NMR (300 MHz, CDCl₃): 5.39-5.32 (m, 4H), 4.06-4.03 (m, 2H), 3.71-3.67 (m, 2H), 3.47-3.46 (m, 1H), 2.01-1.99 (m, 10H), 1.66-1.59 (m, 4H), 1.56-1.54 (m, 6H), 1.34-1.26 (m, 44H), 0.87 (t, J=6.3 Hz, 6H).

### Synthesis of 3-((S)-2,2-di((Z)-hexadec-9-en-1-yl)-1,3-dioxolan-4-yl) propan-1-ol

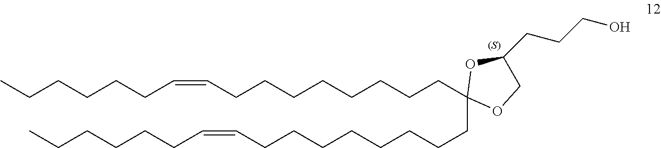

The procedure was previously described.

3-((S)-2,2-di((Z)-hexadec-9-en-1-yl)-1,3-dioxolan-4-yl) propan-1-ol, (0.9 g, 1.56 mmol, 80%) as a clear oil.

¹H NMR (300 MHz, CDCl₃): 5.39-5.28 (m, 4H), 4.06-4.01 (m, 2H), 3.71-3.67 (m, 2H), 3.47-3.46 (m, 1H), 2.01-1.99 (m, 10H), 1.66-1.59 (m, 4H), 1.56-1.54 (m, 6H), 1.34-1.26 (m, 37H), 0.87 (t, J=6.3 Hz, 6H).

### Synthesis of 2-((S)-2,2-di((Z)-octadec-9-en-1-yl)-1,3-dioxolan-4-yl)-N,N-dimethylethan-1-amine, (AKG-KC2-OA, O-11880)

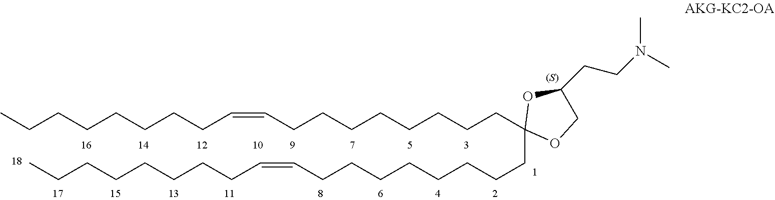

The procedure was previously described.

2-((S)-2,2-di((Z)-hexadec-9-en-1-yl)-1,3-dioxolan-4-yl)-N,N-dimethylethan-1-amine, (AKG-KC2-OA, O-11880), (200 mg, 0.31 mmol, 49%) as a clear oil.

¹H NMR (300 MHz, CDCl₃): 5.38-5.28 (m, 4H), 4.08-4.01 (m, 2H), 3.48 (t, J=6.8 Hz, 1H), 2.39-2.24 (m, 2H), 2.21 (s, 6H), 2.01-1.97 (m, 8H), 1.82-1.77 (m, 2H), 1.68-1.52 (m, 6H), 1.34-1.26 (m, 46H), 0.87 (t, J=6.3 Hz, 6H).

MS(APCI) for C₄₃H₈₃NO₂: 646.7

### Synthesis of 2-((S)-2,2-di((Z)-hexadec-9-en-1-yl)-1,3-dioxolan-4-yl)-N,N-dimethylethan-1-amine, (AKG-KC2-PA, O-11879)

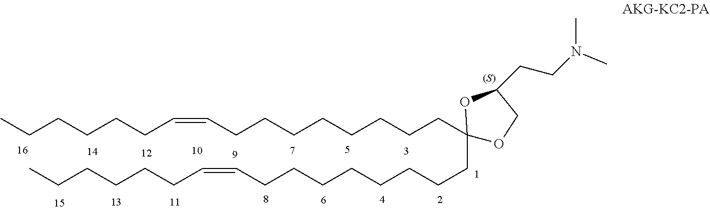

The procedure was previously described.

2-((S)-2,2-di((Z)-hexadec-9-en-1-yl)-1,3-dioxolan-4-yl)-N,N-dimethylethan-1-amine, (AKG-KC2-PA, O-11879), (195 mg, 0.33 mmol, 18%) as a clear oil.

¹H NMR (300 MHz, CDCl₃): 5.35-5.28 (m, 4H), 4.08-4.02 (m, 2H), 3.48 (t, J=6.6 Hz, 1H), 2.38-2.27 (m, 2H), 2.20 (s, 6H), 2.01-1.99 (m, 8H), 1.97-1.80 (m, 2H), 1.77-1.52 (m, 6H), 1.34-1.29 (m, 38H), 0.87 (t, J=6.3 Hz, 6H).

MS(APCI) for C₃₉H₇₅NO₂: 590.6

Synthesis of 3-((S)-2,2-di((Z)-octadec-9-en-1-yl)-1,3-dioxolan-4-yl)-N,N-dimethylpropan-1-amine, (AKG-KC3-OA, O-11957)

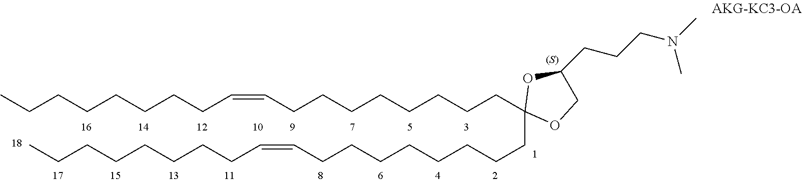

The procedure was previously described.

3-((S)-2,2-di((Z)-octadec-9-en-1-yl)-1,3-dioxolan-4-yl)-N,N-dimethylpropan-1-amine, (AKG-KC3-OA, O-11957), (160 mg, 0.24 mmol, 37%) as a clear oil.

¹H NMR (300 MHz, CDCl₃): 5.39-5.28 (m, 4H), 4.06-4.01 (m, 2H), 3.44 (t, J=6.8 Hz, 1H), 2.26 (t, J=6.8 Hz, 2H), 2.20 (s, 6H), 2.01-1.97 (m, 8H), 1.82-1.77 (m, 2H), 1.60-1.43 (m, 8H), 1.34-1.26 (m, 46H), 0.87 (t, J=6.3 Hz, 6H).

MS(APCI) for C₄₄H₈₅NO₂: 660.6

### Synthesis of 3-((S)-2,2-di((Z)-hexadec-9-en-1-yl)-1,3-dioxolan-4-yl)-N,N-dimethylpropan-1-amine, (AKG-KC3-PA, O-12418)

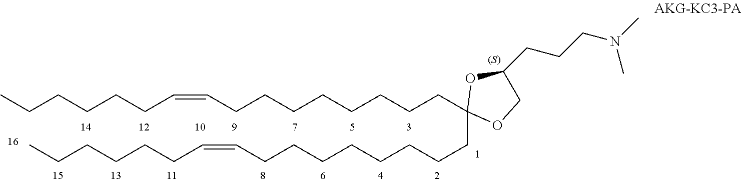

The procedure was previously described.

3-((S)-2,2-di((Z)-hexadec-9-en-1-yl)-1,3-dioxolan-4-yl)-N,N-dimethylpropan-1-amine, (AKG-KC3-PA, O-12418) (300 mg, 0.49 mmol, 32%) as a clear oil.

¹H NMR (300 MHz, CDCl₃): 5.39-5.28 (m, 4H), 4.06-4.01 (m, 2H), 3.47-3.42 (m, 1H), 2.43-2.41 (m, 2H), 2.31 (s, 6H), 2.01-1.97 (m, 8H), 1.70-1.52 (m, 6H), 1.27-1.18 (m, 42H), 0.87 (t, J=6.6 Hz, 6H).

MS(APCI) for C₄₄H₈₅NO₂: 604.6

### Synthesis of 3-((S)-2,2-di((Z)-heptadec-8-en-1-yl)-1,3-dioxolan-4-yl)-N,N-dimethylpropan-1-amine, AKG-KC3-C17 (C8:1)

### Synthesis of(S)-3-(2,2-diheptadecyl-1,3-dioxolan-4-yl)-N,N-dimethylpropan-1-amine, AKG-KC3-C17

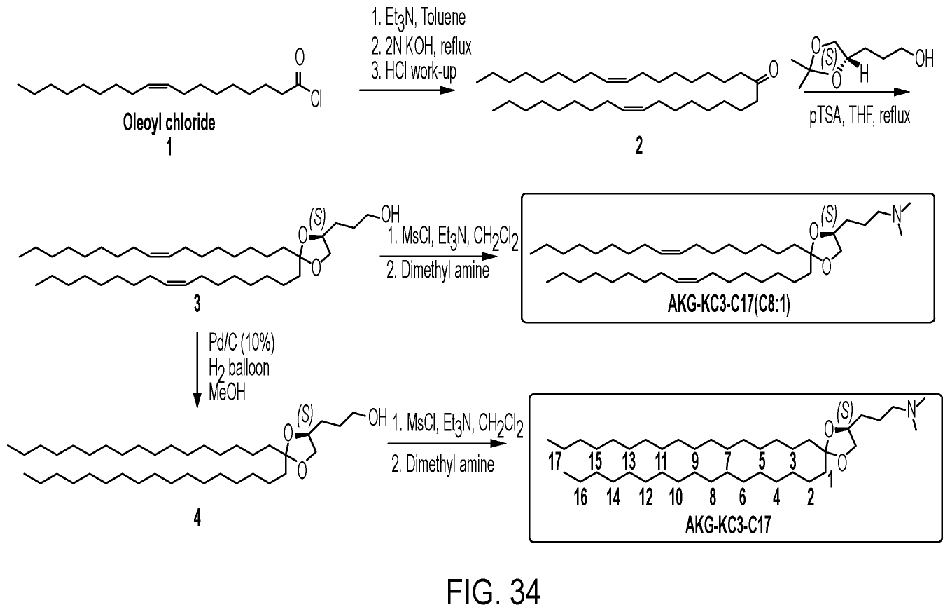

FIG. 34 is a scheme showing the synthesis of AKG-KC3-C17 (C8:1) and AKG-KC3-C17.

### Experimental Procedure

### Synthesis of (9Z,26Z)-pentatriaconta-9,26-dien-18-one, 2

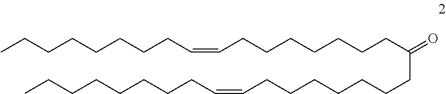 [SMILES: CCCCCCCC/C=C\CCCCCCCC(=O)CCCCCCC/C=C\CCCCCCCC]

To a stirring solution of oleoyl chloride (10 g, 33.3 mmol) in toluene (50 mL) at 0° C. was added triethylamine (5.8 mL, 33.3 mmol). A heavy precipitate formed, and the mixture was allowed to stir at room temperature for 8 hours. The mixture was quenched with 2% sulfuric acid solution and then extracted with ethyl acetate. The organics were washed with brine then dried over magnesium sulfate and filtered. The filtrate was concentrated under vacuum to give a crude oil. The resulting oil was diluted with ethanol (20 mL) and [2N NaOH] (30 mL) was added. The mixture was heated at 100° C. for 12 hours then cooled. The mixture was diluted with 2N HCl solution until a pH 4 was obtained. The mixture was extracted with ethyl acetate. The combined organics were washed with brine then dried over magnesium sulfate and filtered. The filtrate was concentrated under vacuum to give a crude oil. Purification of the oil on silica using 10-20% ethyl acetate in n-hexane as eluant gave (9Z,26Z)-pentatriaconta-9,26-dien-18-one, 2 (3.8 g, 44%) as a yellow oil.

¹H NMR (300 MHz, CDCl₃): δ ppm 5.35-5.31 (m, 4H), 2.39-2.34 (m, 4H), 2.0-1.85 (m, 8H), 1.57-1.52 (m, 4H), 1.27-1.25 (m, 40H), 0.88 (t, J=6.6 Hz, 3H).

### Synthesis of 3-((S)-2.2-di((Z)-heptadec-8-en-1-yl)-1,3-dioxolan-4-yl) propan-1-ol, 3

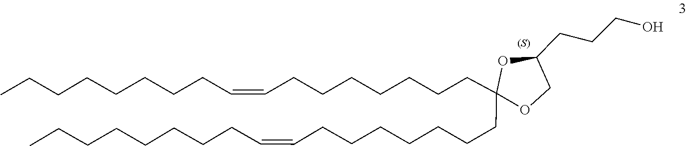

Procedure previously described synthesis of AKG-KC2-01

3-((S)-2,2-di((Z)-heptadec-8-en-1-yl)-1,3-dioxolan-4-yl) propan-1-ol, 3 (0.7 g, 1.15 mmol, 65%) as a clear oil.

¹H NMR (300 MHz, CDCl₃): δ ppm 5.38-5.31 (m, 4H), 4.08-4.02 (m, 2H), 3.67-3.66 (m, 2H), 3.48-3.43 (m, 1H), 2.15-2.13 (m, 1H), 2.00-1.98 (m, 8H), 1.65-1.56 (m, 8H), 1.27-1.25 (m, 44H), 0.88 (t, J=6.6 Hz, 6H).

### Synthesis of(S)-3-(2,2-diheptadecyl-1,3-dioxolan-4-yl)propan-1-ol, 4

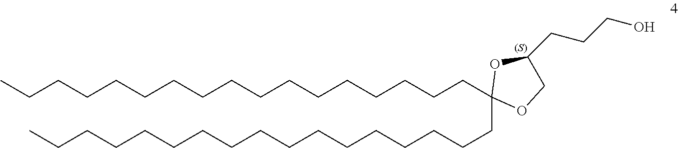

A solution of 3-((S)-2,2-di((Z)-heptadec-8-en-1-yl)-1,3-dioxolan-4-yl) propan-1-ol (1.3 g, 2.15 mmol) in methanol/ethyl acetate (20 mL, 1:1/v:v) was hydrogenated at 1 atm (hydrogen balloon) over 10% palladium on carbon (100 mg) for 2 hours. The mixture was evacuated of hydrogen and flowed with nitrogen. The mixture was filtered over celite, and the filtrate concentrated under vacuum to give(S)-3-(2,2-diheptadecyl-1,3-dioxolan-4-yl)propan-1-ol, 4 (1.3 g, quant.) as a clear oil.

¹H NMR (300 MHz, CDCl₃): δ ppm 4.10-4.01 (m, 2H), 3.71-3.64 (m, 1H), 3.47-3.43 (m, 2H), 2.05-2.01 (m, 1H), 1.63-1.51 (m, 8H), 1.42-1.22 (m, 58H), 0.87 (t, J=6.6 Hz, 6H).

### Synthesis of 3-((S)-2,2-di((Z)-heptadec-8-en-1-yl)-1,3-dioxolan-4-yl)-N,N-dimethylpropan-1-amine, AKG-KC3-C17 (C8:1)(O-12620)

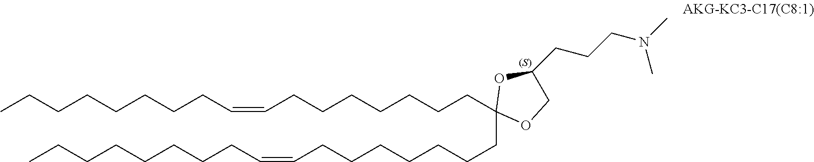

Procedure previously described synthesis of AKG-KC2-01

3-((S)-2,2-di((Z)-heptadec-8-en-1-yl)-1,3-dioxolan-4-yl)-N, N-dimethylpropan-1-amine, (AKG-KC3-C17 (C8:1), O-12620), (290 mg, 0.46 mmol, 40%) a clear oil.

MS (APCI⁺) for C₄₂H₈₁NO₂: 632.6

¹H NMR (300 MHz, CDCl₃): δ ppm 5.38-5.28 (m, 4H), 4.09-3.99 (m, 2H), 3.48-3.41 (m, 1H), 2.77-2.71 (m, 1H), 2.55 (s, 6H), 2.01-1.95 (m, 8H), 1.88-1.78 (m, 2H), 1.62-1.50 (m, 6H), 1.27-1.24 (m, 44H), 0.88 (t, J=6.8 Hz, 6H).

### Synthesis of (S)-3-(2,2-diheptadecyl-1,3-dioxolan-4-yl)-N,N-dimethylpropan-1-amine, AKG-KC3-C17 (O-12637)

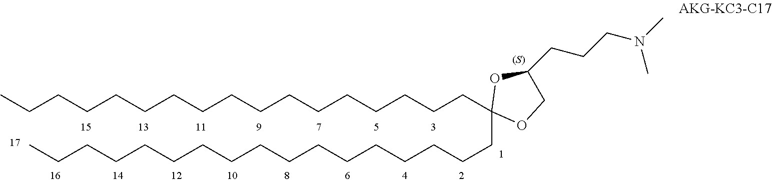

Procedure previously described synthesis of AKG-KC2-01

(S)-3-(2,2-diheptadecyl-1,3-dioxolan-4-yl)-N, N-dimethylpropan-1-amine, (AKG-KC3-C17, O-12637), (275 mg, 0.43 mmol, 22%) as a solid.

MS(APCI⁺) for C₄₂H₈₅NO₂: 636.6

¹H NMR (300 MHz, CDCl₃): δ ppm 4.06-4.00 (m, 2H), 3.47-3.43 (m, 1H), 2.29-2.25 (m, 2H), 2.21 (s, 6H), 1.60-1.48 (m, 8H), 1.29-1.24 (m, 60H), 0.87 (t, J=6.6 Hz, 6H).

### Example 2. Preparation of Lipidic Nanoparticles (LNPs)

mRNA modified with 5-methoxyuridine (5moU) and coding for mCherry (Cat #L-7203) was obtained from Trilink Biotechnologies (San Diego, CA). All uridine nucleosides were substituted with N1-methyl-pseudouridine. To produce the mRNA, a synthetic gene encoding the mRNA sequence was cloned into a DNA plasmid. The synthetic gene was comprised of an RNA promoter, a 5′ untranslated region, mCherry protein coding sequence, a 3′ untranslated region, and a poly(A) tail region of approximately 120 As. The open reading frame sequence for the mCherry mRNA from TriLink (Cat #L-7203) corresponds to SEQ ID NO: 227:

AUGGUGAGCAAGGGCGAGGAGGACAACAUGGCCAUCAUCAAGGAG
UUCAUGCGGUUCAAGGUGCACAUGGAGGGCAGCGUGAACGGCCAC
GAGUUCGAGAUCGAGGGCGAGGGCGAGGGCCGGCCCUACGAGGGC
ACCCAGACCGCCAAGCUGAAGGUGACCAAGGGCGGCCCCCUGCCC
UUCGCCUGGGACAUCCUGAGCCCCCAGUUCAUGUACGGCAGCAAG
GCCUACGUGAAGCACCCCGCCGACAUCCCCGACUACCUGAAGCUG
AGCUUCCCCGAGGGCUUCAAGUGGGAGCGGGUGAUGAACUUCGAG
GACGGCGGCGUGGUGACCGUGACCCAGGACAGCAGCCUGCAGGAC
GGCGAGUUCAUCUACAAGGUGAAGCUGCGGGGCACCAACUUCCCC
AGCGACGGCCCCGUGAUGCAGAAGAAGACCAUGGGCUGGGAGGCC
AGCAGCGAGCGGAUGUACCCCGAGGACGGCGCCCUGAAGGGCGAG
AUCAAGCAGCGGCUGAAGCUGAAGGACGGCGGCCACUACGACGCC
GAGGUGAAGACCACCUACAAGGCCAAGAAGCCCGUGCAGCUGCCC
GGCGCCUACAACGUGAACAUCAAGCUGGACAUCACCAGCCACAAC
GAGGACUACACCAUCGUGGAGCAGUACGAGCGGGCCGAGGGCCGG
CACAGCACCGGCGGCAUGGACGAGCUGUACAAGAGCGGCAACUGA

Stock solutions of each lipid were prepared. Ionizable lipids were weighed out in 4 mL glass vials (Thermo B7999-2) and dissolved in ethanol (Sigma-Aldrich 200 proof, RNase free) to a final concentration of 10 mM. Other lipids such as DSPC, DPPC-NH₄, Cholesterol and PEG-DMG were weighed out and dissolved in ethanol to a concentration of 1 mM. DSPS-Na was dissolved in methanol (Sulpelco, Omnisolve) at a concentration of 1 mM and briefly heated to 70° C. to complete its dissolution.

Lipid mixtures for each individual LNP were prepared by adding the desired volume of each lipid stock solution to a new vial, adding ethanol if needed to achieve a final volume of 1.2 mL. For example, an LNP formulation of AKG-UO-1/DSPC/DSPS/Chol/PEG-DMG (50/2.5/7.5/38.5/1.5 mol %), with an N/P of 5 contained 1500 nmol AKG-UO-1, 75 nmol DSPC, 225 nmol DSPS, 1155 nmol Chol and 45 nmol PEG-DMG for every 100 μg of mRNA used.

mRNA solutions were prepared by thawing frozen mRNA (mCherry mRNA, Trilink) vials and diluting mRNA in 6.25 mM sodium acetate (pH 5.0) to a final concentration of 0.033 mg/mL. To prepare LNPs, a NanoAssemblr Benchtop microfluidic device (from Precision Nanosystems) was used. If LNPs contained the sodium or ammonium salts of DSPS, or sodium salt of DPPS the heating block accessory set to 70° C. was used, otherwise LNPs were mixed at room temperature. 3 mL of mRNA solution was loaded into a 3 mL disposable syringe (BD 309656) and 1 ml of lipid mixture in a 1 ml syringe (BD309659) and placed in the NanoAssemblr heating block for 4 min prior to mixing. LNP formation was achieved by pumping the liquid streams through a disposable microfluidics cassette at 3:1 aqueous: alcohol volume ratio at 6 mL/min mixing speed. After mixing, 3.6 mL of LNP mixture was collected, while the initial mixed volume of 0.35 mL and last 0.05 mL of mix was discarded. Ethanol was removed by buffer exchange using SpectraPor dialysis tubing (12-14k MWCO) in PBS (Cytivia, SH30256.01) or by sequential concentration and dilution using Amicon Ultra-4 centrifugal concentrators (10k MWCO, at 500 g).

LNPs were typically exchanged into PBS, pH 7.4 and then 15 mM Tris, pH 7.4, 20% sucrose, concentrated to 20-50 ug/mL mRNA using an Amicon-Ultra 4 (100,000 MWCO) spin column, sterile filtered (Thermo Nalgene 0.2 um #720-1320) prior to freezing by immersion in liquid nitrogen for 5 min and long-term storage at −80° C.

### Example 3. LNP Characterization

A. mRNA Concentration and Relative Encapsulation Efficiency Determination by Fluorescent Binding Dye

Materials: Ribogreen reagent (Thermo #11491), 3×96-well plates with lids, PBS, dissociation buffer (PBS with 10% DMSO and 1% (wt/wt) Zwittergent 3-14 (Sigma-Aldrich #693017), mRNA, general pipette tips & repeater pipette tips.
1. 5 mL of 2 μg/mL mRNA stock were prepared in DPBS or PBS
2. Diluted standards were prepared as follows in single wells in a 96-well plate (Plate A);

| Final [mRNA] ng/mL | Vol. stock 2 μg/mL (μL) | Vol. PBS (μL) |
|---|---|---|
| 2000 | 400 | 0 |
| 1500 | 300 | 100 |
| 1000 | 200 | 200 |
| 500 | 100 | 300 |
| 0 | 0 | 400 |

3. Using different wells in Plate A, sample mRNA concentration was estimated and were diluted to be within the standard curve. For example, if the approximate mRNA concentration should be ~30 μg/mL in the sample, a 20× dilution was performed (Dilution Factor). (20 μL sample added to 380 μL PBS in a well). No lid was used on plate A. Samples were mixed by gentle pipetting up & down.

### Example of Plate A

| | | | | | | | | | |
|---|---|---|---|---|---|---|---|---|---|
| A | 0 | 500 | 1000 | 1500 | 2000 | | | | |
| B | | | | | | | | | |
| C | S 1 | S 2 | S 3 | S 4 | S 5 | S 6 | S 7 | S 8 | Etc. |
| D | | | | | | | | | |
| E | | | | | | | | | |
| F | | | | | | | | | |
| G | | | | | | | | | |
| H | | | | | | | | | |

4. Two more plates, plates B & C were used. Using a multichannel pipettor, 60 μL of each standard 2 were pipetted into wells each (duplicate), and sample into 3 wells each (triplicate)

### Example of Plate B and C

| | | | | | | | | | |
|---|---|---|---|---|---|---|---|---|---|
| A | 0 | 500 | 1000 | 1500 | 2000 | | | | |
| B | 0 | 500 | 1000 | 1500 | 2000 | | | | |
| C | S 1 | S 2 | S 3 | S 4 | S 5 | S 6 | S 7 | S 8 | Etc. |
| D | S 1 | S 2 | S 3 | S 4 | S 5 | S 6 | S 7 | S 8 | Etc. |
| E | S 1 | S 2 | S 3 | S 4 | S 5 | S 6 | S 7 | S 8 | Etc. |
| F | | | | | | | | | |
| G | | | | | | | | | |
| H | | | | | | | | | |

5. The number of wells used on each plate was counted and 4 was added to this number. For plate B, PBS was prepared with Ribogreen diluted 1:100. For example, for 40 wells, 44 was used as the number. 44×60 μL=2.64 mL Ribogreen solution needed, so that would be 2.61 mL PBS with 26.4 μL Ribogreen.
6. For plate C, 2.61 mL Dissociation buffer and 26.4 uL Ribogreen was pipetted.
7. Using a repeater pipette set for 60 μL, PBS+RiboGreen was added to each well on plate B and 60 μL Dissociation Buffer+Ribogreen to plate C. Both plates B and C were mixed on an orbital mixer (120 rpm) for 1 min. Plate B was placed in the dark for 15 min. Plate C was incubated at 37° C. in the dark for 10 min, followed by 5 min at RT.
8. Both plates were read one after the other, using Ex. 465, Em. 530 nm
9. Using the standard curve, the slope and intercept were calculated and by extrapolation the mRNA concentrations of the samples on plate B & C were calculated (average and std.dev)
10. Percent encapsulation efficiency (% EE) by [mRNA] plate B/[mRNA] plate C×100 was calculated
11. Total [mRNA] by taking [mRNA] plate C X dilution factor was calculated.
   b. LNP Particle size
1. 30 μL of LNP was mixed with 1.5 mL PBS in a polystyrene cuvette (Sarstedt, #67.754) and analyzed for size using a ZetaSizer Pro (Malvern) using ZS Xplorer software, version number 1.4.0.105. The Z-average size and polydispersity index value were recorded. Typically, size measurements of LNPs were taken post LNP mixing, post buffer exchange and post sterile filtering.

### C. LNP Zeta Potential

1. 30 μL of LNP was mixed with 1.5 mL PBS and injected into a disposable folded capillary cell (Malvern Nanoseries DTS1070) and zeta potential measured on a ZetaSizer Pro at 25° C.

### Example 4. Determination of Transfection Efficiency in Murine Dendritic Cells of LNPs Using mCherry mRNA

### A. Cell Propagation, Transfection, Harvesting and Staining Protocol

1. MutuDC1940 cells (Applied Biological Materials, T0528) were grown according to supplier's instructions in T75 flasks. They were plated at 180,000 cells/well into 24-well plate one day prior to transfection.
2. LNPs were added in triplicate to each well at the desired mRNA concentration (e.g. 1 μg/mL) in 1 mL media and after 24 h the cells were washed once with DPBS (VWR 02-0119-1000).
3. 0.2 mL of DPBS (plus 5 mM EDTA, pH 7.4) was then added to facilitate detachment.
4. The cells were placed at 37° C. for 3 min, until detached.
5. 0.5 ml DPBS added to each well and the liquid transferred to a flow cytometry tube (Falcon 5 mL #352054).
6. The tube was centrifuged at 1100 rpm for 3-5 min and the liquid poured off.
7. 100 μL of Zombie Violet (Biolegend) (diluted 1:500 in PBS) was added to each tube.
8. The tubes were gently tapped to resuspend cells and placed in the dark for 15 min at RT.
9. To the cells 0.5 mL of (paraformaldehyde 4% in PBS: DPBS 1:1) was added and the cells flicked gently to resuspend and put on ice for 30 min. Another 2 ml PBS was added.
10. The cells were pelleted as above and resuspended in 0.5 mL DPBS with 5% BSA and placed in the fridge until needed.

### B. Cell Analysis

Cells suspensions were analyzed by an Attune NxT flow cytometer using the VL1 and YL2 for live/dead and mCherry fluorescence signals respectively. Gating analysis was performed on FloJo software.

### Example 5. Impact of DSPS on Transfection Efficiency of Dendritic Cells Using LNPs with KC2 as Ionizable Cationic Lipid

The aim of this study was to explore the effect of phosphatidylserine targeting using DSPS on transfection efficiency in murine dendritic cells. LNPs were prepared as described in Example 9, characterized for particle size and zeta potential as described in Example 4, and evaluated for transfection efficiency in murine dendritic cells as described in Example 5. The LNPs all had DLin-KC2-DMA constant at an N/P ratio of 5 and 50 mol % of total lipid, the PS lipid was varied initially from 0-2.5 mol % and the DSPC phospholipid varied from 0-7.5 mol % (Total mol % of DSPC and DSPS was constant at 10 mol %), and the cholesterol constant at 38.5 mol % (all mol % of total lipid). The particle size, Polydispersity Index (PDI), and entrapment efficiency for all formulations is shown below in Tables 4 and 5.

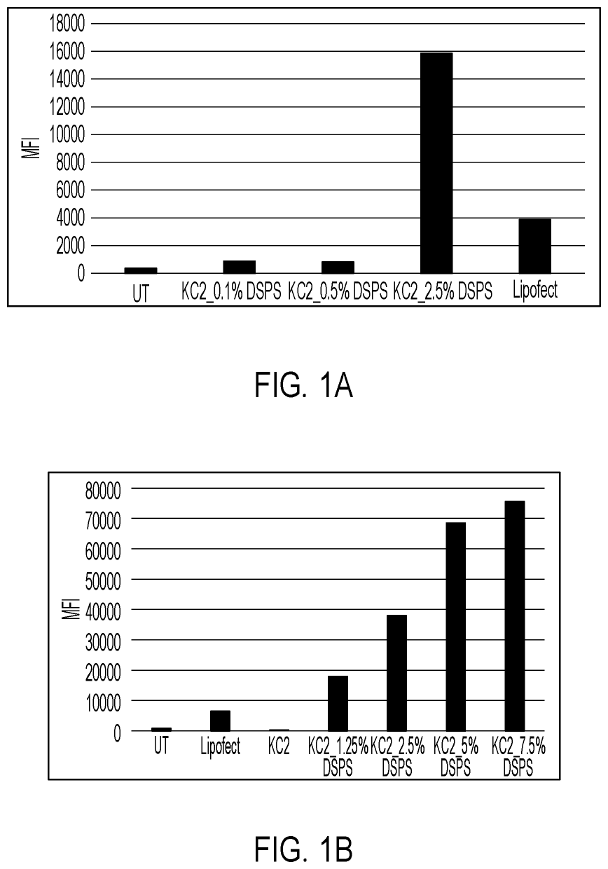

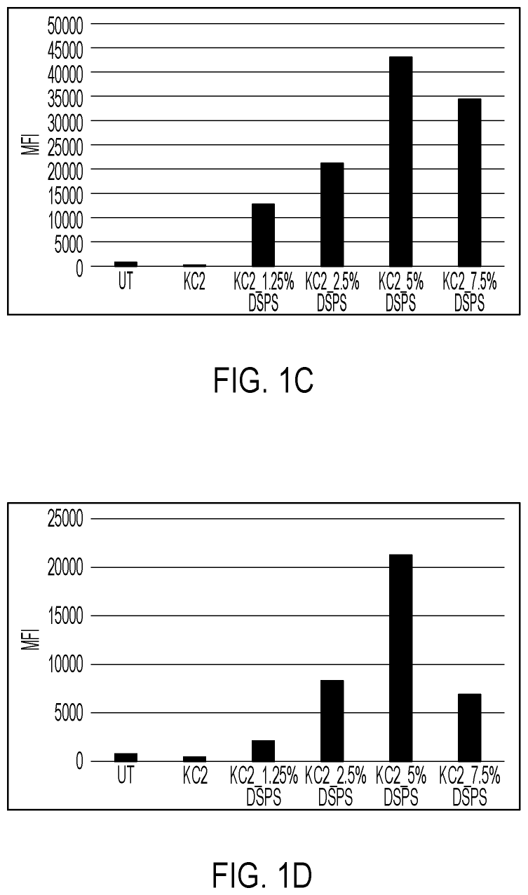

TABLE 4
Physicochemical properties of KC2-containing LNPs used in Example
6 varying from 0-2.5 mol % used in Example 6 and FIG. 1A.

| Mol % DSPS | Particle Size (nm) | PDI | % Encapsulation ± SD |
|---|---|---|---|
| 0 | 77.6 | 0.075 | 86.4 ± 2.8 |
| 0.1 | 82.0 | 0.171 | 87.0 ± 4.7 |
| 0.5 | 89.2 | 0.285 | 86.3 ± 3.2 |
| 2.5 | 72.2 | 0.263 | 87.1 ± 6.8 |

TABLE 5
Physicochemical properties of KC2-containing LNPs varying from 0-
7.5 mol % used in Example 6 and FIG. 1B, FIG. 1C, and FIG. 1D.

| Mol % DSPS | Particle Size (nm) | PDI | % Encapsulation ± SD |
|---|---|---|---|
| 0 | 73.9 | 0.14 | 92.6 ± 6.2 |
| 1.25 | 80.3 | 0.13 | 90.9 ± 5.6 |
| 2.5 | 70.7 | 0.25 | 89.0 ± 7.4 |
| 5 | 78.7 | 0.12 | 85.1 ± 6.8 |
| 7.5 | 76.4 | 0.14 | 85.4 ± 3.6 |

An initial set of LNPs containing DLin-KC2-DMA and varying phosphatidylserine in the form of DSPS from 0-2.5 mol %, showed little transfection at 0 or 0.5 mol % DSPS, but increase by 18-fold when DSPS was incorporated at 2.5 mol % (FIG. 1A). A second series of LNPs prepared with DSPS from 0-7.5 mol % was evaluated at 0.1, 0.3, and 1 μg/mL mRNA concentrations (FIG. 1B, FIG. 1C and FIG. 1D). The transfection efficiency increased as the mol % of DSPS was increased above 2.5 mol %, with a maximum at 7.5 mol % at 1 μg/mL mRNA, and 5 mol % at both 0.1 and 0.3 μg/mL mRNA. These data demonstrate that the inclusion of phosphatidyl-L-serine can dramatically increase the transfection efficiency of mRNA-containing LNPs, and that maximal uptake occurs between 5-7.5 mol % of DSPS (as % of total lipid).

### Example 6. Impact of ICL and Anionic Phospholipid Targeting Ligand on mRNA Transfection of Dendritic Cells

The aim of this study was to see if other anionic phospholipids could also enhance the transfection efficiency of LNPs and how LNPs prepared with varying ICLs and PS targeting would transfect dendritic cells. LNPs were prepared as described in Example 2, characterized for particle size and zeta potential as described in Example 3, and evaluated for transfection efficiency in murine dendritic cells as described in Example 4. The LNPs had various ICLs (DLin-KC2-DMA, KC2-OA, KC3-OA, or SM-102) constant at an N/P ratio of 5 and 50 mol % of total lipid, the PS lipid was kept constant at 5 mol % and the DSPC at 5 mol %, and the cholesterol constant at 38.5 mol % (all mol % of total lipid). The particle size, PDI, and entrapment efficiency for all formulations is shown below in Table 6.

TABLE 6
Physicochemical properties of LNPs varying in ICL
used and with anionic phospholipid at 5 mol %.

| Ionizable | | Particle | | % |
|---|---|---|---|---|
| Cationic | Anionic | Size | | Encapsulation ± |
| Lipid (ICL) | lipid | (nm) | PDI | SD |
| KC2 | 5% DSPS | 90.9 | 0.13 | 91.0 ± 5.8 |
| KC2-OA | 5% DSPS | 74.8 | 0.13 | 92.8 ± 8.0 |
| KC3-0A | 5% DSPS | 95.5 | 0.09 | 96.6 ± 18.3 |
| SM-102 | None | 77.5 | 0.05 | 92.6 ± 5.0 |
| SM-102 | 5% DSPS | 65.9 | 0.13 | 84.5 ± 7.7 |
| KC2 | 5% Glu-DSPE | 52.1 | 0.23 | 87.9 ± 6.8 |
| KC2 | 5% Suc-DSPE | 64.6 | 0.19 | 86.6 ± 3.6 |

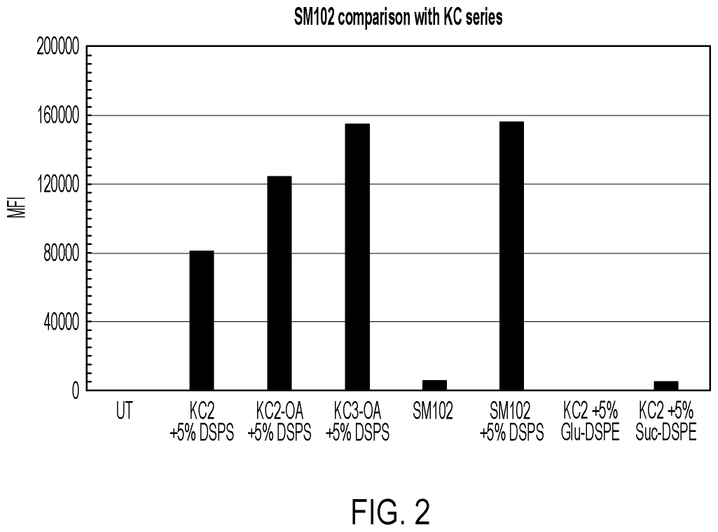

The transfection results are shown in FIG. 2 show high transfection rates with three different KC-series ICLs (KC2, KC2-OA, and KC3-OA), and also with LNPs prepared with the branched ICL, SM-102. The encapsulation efficiency was high and the particle size below 100 nm for all formulations, including those prepared with alternate anionic phospholipids (Suc-DSPE or Glu-DSPE). The data demonstrate that DSPS (L-serine) can not be substituted with either N-glutaryl-distearoylphosphatidylethanolamine (Glu-DSPE) or N-succinyl-distearoylphosphatidylethanolamine (Suc-DSPE) and provide the same high level of mRNA transfection despite both phospholipids also containing two negative charges and both containing the same distearoyl (C18:0) fully saturated acyl chains. These studies also clearly show that the addition of DSPS can give rise to high transfection efficiencies for other ionizable cationic lipids, including those with a single unsaturated acyl gain (KC2-OA or KC3-OA) and those including a branched ICL, like SM-102. The addition of DSPS to SM-102 containing LNPs gave rise to a 22-fold increase in mCherry expression, for example.

### Example 7. Dependence of PS Targeting on ICL and PS Structure

The aim of this study was to compare PS-targeted LNPs with KC2 and KC3 series ionizable cationic lipids of varying acyl chain composition. KC2 series lipids having a structure of dimethylaminoethyl headgroup structure were compared to the KC3 series containing a dimethylaminopropyl-derivatized head group. The LNPs contained various ICLs (KC2, KC2-01, KC2-OA, KC2-PA, KC3-OA, and KC3-01) as the ICL at an N/P ratio of 5 and 50 mol % ICL, and a constant 1.5 mol % PEG-DMG. The cholesterol content was held constant at 38.5 mol % and the DSPC content varied inversely with the mol % of DSPS at either 0 or 5 mol % (all lipid concentrations were used as mol % of total lipid). Transfection efficiency was evaluated in murine dendritic cells as described in Example 4.

TABLE 7
Physicochemical properties of LNPs varying in ICL
used and with anionic phospholipid at 5 mol %.

| Ionizable | | Particle | | % |
|---|---|---|---|---|
| Cationic | | Size | | Encapsulation ± |
| Lipid (ICL) | PS content | (nm) | PDI | SD |
| KC2 | None | 95.6 | 0.08 | 91.7 ± 2.0 |
| KC2 | 5% DSPS | 82.5 | 0.09 | 87.1 ± 3.8 |
| KC2-01 | None | 82.4 | 0.08 | 91.6 ± 3.9 |
| KC2-01 | 5% DSPS | 76.0 | 0.10 | 90.5 ± 3.5 |
| KC2-OA | None | 89.5 | 0.08 | 88.6 ± 4.2 |
| KC2-OA | 5% DSPS | 85.9 | 0.10 | 92.4 ± 4.5 |
| KC2-PA | None/10% DPPC | 92.3 | 0.11 | 92.4 ± 4.4 |
| KC2-PA | 5% DSPS/5% DPPC | 85.2 | 0.10 | 95.5 ± 3.4 |
| KC3-01 | None | 88.2 | 0.10 | 94.3 ± 7.1 |
| KC3-01 | 5% DSPS | 101.2 | 0.16 | 95.2 ± 4.4 |
| KC3-OA | None | 105.9 | 0.08 | 86.6 ± 1.6 |
| KC3-OA | 5% DSPS | 103.0 | 0.13 | 93.8 ± 3.2 |
| KC2-PA | 5% DPPS/5% DPPC | 77.1 | 0.06 | 93.8 ± 3.7 |

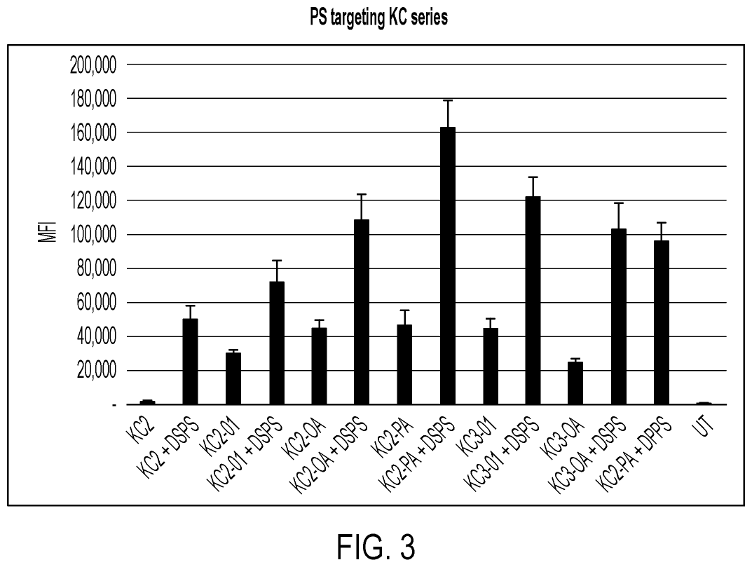

The transfection results are shown in FIG. 3 and clearly show a positive impact of PS targeting on multiple KC-series ICLs. Here, the data show that ICLs containing both unsaturated C16 and C18 ICLs could be targeted with phosphatidyl-L-serine and give rise to high transfection rates for dendritic cells. The highest rate of transfection came when the PS and PC contained a mismatched acylchain composition, with 5 mol % DPPC and 5 mol % DSPS, and combined with a C16 ICL (KC2-PA).

### Example 8. Impact of PEG on Transfection Efficiency of AKG-UO-1 Containing LNPs

The aim of this study was to explore the impact of PEG-lipid density on transfection efficiency of nontargeted and phosphatidyl-L-serine targeted LNPs. LNPs were prepared as described in Example 2. The LNPs contained AKG-UO-1 as the ICL at an N/P ratio of 5 with either 0 or 5 mol % DSPS and between 0.5-4.5 mol % PEG-DMG. The cholesterol content was held constant at 38.5 mol % and the DSPC content was 10 mol % for the formulations with no DSPS and 5 mol % for those with 5 mol % DSPS. At PEG-DMG content above 1.5 mol %, the total cholesterol content was reduced by the amount of PEG-DMG added, for example with a PEG-DMG content of 3.5 mol %, the cholesterol content was reduced to 36.5 mol % from 38.5 mol %. The particles with 0.5% PEG-DMG showed a negative zeta potential at pH 7.4, and a significant shift to a positive zeta potential at pH 5. The LNPs with 1.5-3.5 mol % PEG-DMG were essentially neutral at pH 7.

TABLE 8
Physicochemical properties of LNPs used in Example
8 and containing AKG-UO-1, 0 or 5 mol % DSPS

| % | % | Particle | Zeta Potential | Zeta Potential |
|---|---|---|---|---|
| DSPS | PEG-DMG | Size (nm) | at pH 7 (mV) | at pH 5 (mV) |
| 0 | 0.5 | 98.9 | 0.27 | 25.92 |
| 0 | 1.5 | 75.0 | −0.87 | 12.07 |
| 0 | 2.5 | 73.8 | −0.51 | 11.86 |
| 0 | 3.5 | 81.0 | −0.30 | 6.34 |
| 0 | 4.5 | 143 | −1.47 | 14.51 |
| 5 | 0.5 | 106.1 | −1.35 | 9.30 |
| 5 | 1.5 | 73.7 | −0.21 | 3.23 |
| 5 | 2.5 | 70.7 | −0.20 | 4.75 |
| 5 | 3.5 | 65.6 | −0.10 | 7.54 |
| 5 | 4.5 | 63.1 | −1.12 | 3.73 |

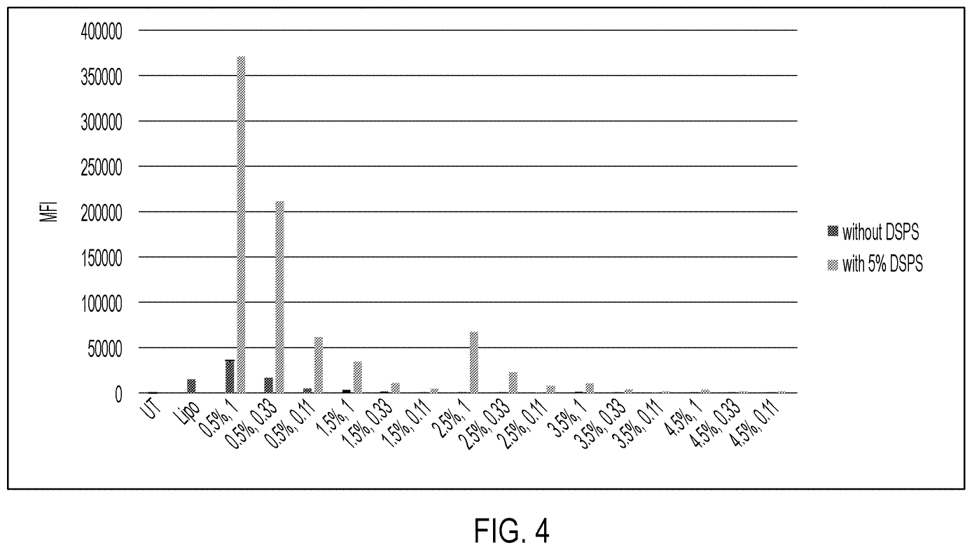

The impact of PEG-DMG on transfection of dendritic cells by DSPS-targeted LNPs containing the AKG-UO-1 ICL is shown in FIG. 4. The formulation with 0.5% PEG-DMG showed a transfection efficiency in the presence of 5% DSPS that was between 6-7 fold higher than observed at 1.5-2.5% PEG-DMG. Transfection at 1.5 and 2.5% PEG-DMG was similar, but decreased dramatically at 3.5 and 4.5 mol % PEG-DMG. The ratio of targeted to nontargeted transfection at each PEG-density varied, and was 12-fold at 0. 5% PEG, 7-fold at 1.5% PEG, 37-fold at 2.5% PEG, and below 5-fold at 3.5 and 4.5% PEG, likely because of high PEG-shielding of the PS targeting moiety. The combination of these data show that the optimum range of PEG-densities is between 0.5-2.5% PEG-DMG, with the lower end of the range being optimum for overall transfection efficiency, while the 2.5% being optimal for target specificity.

### Example 9. Impact of N/P Ratio on Transfection Efficiency of mCherry mRNA Containing LNPs

The aim of this study was to explore the effect of different N/P ratios on transfection efficiency in dendritic cells. LNPs were prepared as described in Example 2, characterized for particle size and zeta potential as described in Example 3, and evaluated for transfection efficiency in murine dendritic cells as described in Example 4. The LNPs all used KC2-01 as the ICL but varied the cationic lipid-to-mRNA phosphate (N/P) ratio from 4-7, the PS lipid was constant at 5 mol % and the DSPC phospholipid constant at 5 mol %, and the cholesterol constant at 38.5 mol %. The entrapment efficiency for all formulations was between 84 and 90%, indicating high efficiency mRNA entrapment in the LNP. Transfection efficiency was evaluated in murine dendritic cells as described in Example 4.

TABLE 9
Physicochemical properties of LNPs used in Example 9

| | | Particle | Zeta Potential | Zeta Potential |
|---|---|---|---|---|
| % DSPS | N/P | Size (nm) | at pH 7 (mV) | at pH 5 (mV) |
| 0 | 4 | 85.4 | −10.8 | 16.3 |
| 0 | 5 | 84.9 | −6.9 | 20.9 |
| 0 | 6 | 81.1 | −7.0 | 21.6 |
| 0 | 7 | 75.7 | −6.1 | 21.5 |
| 5 | 4 | 84.1 | −17.5 | 13.8 |
| 5 | 5 | 80.9 | −17.2 | 20.4 |
| 5 | 6 | 82.2 | −15.5 | 15.8 |
| 5 | 7 | 79.5 | −13.9 | 16.6 |

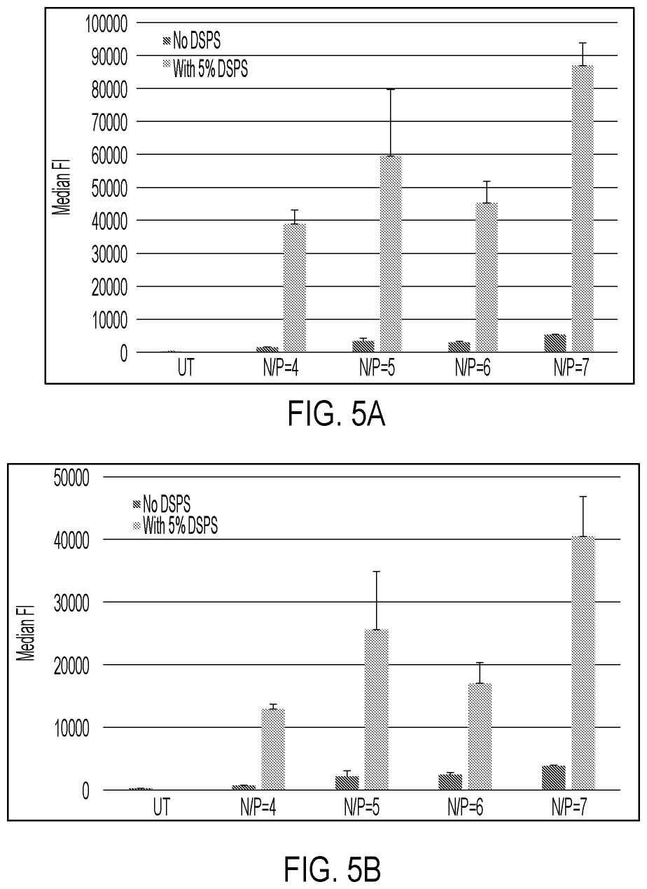

Transfection activity was evaluated for both DSPS-targeted and nontargeted KC-01 LNP formulations at both 1 μg/ml (FIG. 5A) and 0.33 μg/ml (FIG. 5B). These data show high DSPS-mediated transfection efficiency for KC2-01 containing LNPs over a broad range of N/P ratios with the greatest transfection efficiency being observed at an N/P of 7.

### Example 10. Measuring the Effect of Adding 10 and 25 Mol % DOPS on LNP Particle Formation and Activity in MutuDC1940 Dendritic Cell Line

The aim of this study was to explore the impact of including DOPS into mRNA LNP formulations at compositions at or below the mol % previously shown in the literature (Gaitonde et al. (2011) Clin Immunol 138, 135-145; Rodriquez-Fernandez (2018) Front Immunol 9, 253) to enhance liposome (with) uptake into dendritic cells. LNPs were prepared as described in Example 2 at 25° C. and analyzed as in Example 3. The LNPs contained KC2 as the ICL at an N/P ratio of 5 with between 0, 10 and 25 mol % DOPS and a constant 1.5 mol % PEG-DMG. The cholesterol content was held constant at 38.5 mol % for the 0% and 10% DOPS formulations and the DSPC content varied inversely with the mol % of DOPS between 0-10 mol % (all lipid concentrations were used as mol % of total lipid). In the 25 mol % DOPS formulation, there was no DSPC and the cholesterol content decreased in the total by 15 mol % (from 38.5 to 23.5 mol %). Transfection efficiency was evaluated in murine dendritic cells as described in Example 4.

TABLE 10
Physicochemical properties of DOPS containing LNP formulations

| | Particle | | Encapsulation |
|---|---|---|---|
| LNP Formulation | Size (nm) | PDI | Efficiency (%) |
| KC2/DSPC/DOPS/Chol/PEG- | 68.8 | 0.136 | 90.0 ± 3.5 |
| DMG (50/10/0/38.5/1.5) | | | |
| KC2/DSPC/DOPS/Chol/PEG- | 80.8 | 0.163 | 90.9 ± 3.5 |
| DMG (50/0/10/38.5/1.5) | | | |
| KC2/DSPC/DOPS/Chol/PEG- | Peak 1 = 62.6, | 0.398 | 77.3 ± 3.5 |
| DMG (50/0/25/23.5/1.5) | Peak 2 = 416.3 | | |

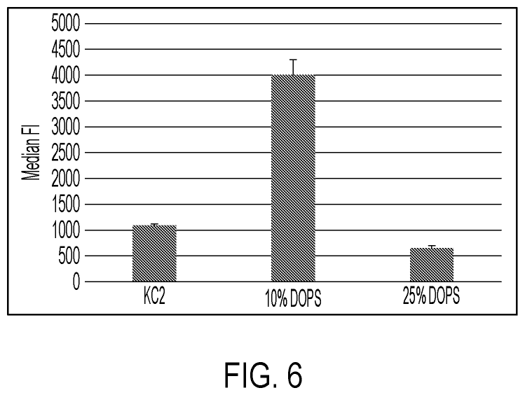

The impact of 10-25 mol % DOPS on targeting of LNPs to dendritic cells was evaluated in FIG. 6. This study shows that including 10 mol % DOPS in a KC2-based LNP formulation had a positive effect on mCherry expression levels in MutuDC1940 cells, while not adversely affecting either particle size or encapsulation of mRNA. However, when the DOPS content was increased to 25 mol % the expression of mCherry was lower than the formulation that had no DOPS and the size distribution widened as demonstrated by an increase in the PDI and gave rise to a distribution that contained particles >400 nm. Taken together, 25 mol % may have been shown in the literature to enhance liposome uptake into dendritic cells, while in an LNP formulation with mRNA it had a deleterious effect on both particle size and transfection activity. Importantly, the DOPS used here and in the literature contained unsaturated acyl chains, in this case oleic acid. This is similar to what is typical in many cells, where the phosphatidylserine acyl chains are often unsaturated in the sn-2 position, in many instances with multiple olefins (2-4). Although there is a small enhancement with a lower concentration of DOPS, this enhancement was shown to be significantly higher when the PS was comprised of saturated acyl chains, most preferably dipalmitoyl (C16) or distearoyl (C18).

### Example 11. Impact of Pegylation and Phosphatidylserine Targeting on Immunogenicity of SARS-COV-2 Spike Protein mRNA Vaccine Constructs

Mice and study design. The in vivo study was carried out. Female BALB/c mice were purchased from Jackson Labs, allowed to acclimate in the vivarium for at least 7 days, and were 6-8 weeks at the start of the study. On study day 0 mice were injected intramuscularly in the right quadricep with 1 ug of vaccine candidate (quantity refers to mRNA) in a volume of 50 μL. Study groups consisted of 5 mice and included vehicle control, comparator vaccines, and experimental vaccine candidates. Mice were given a second injection of the same vaccine candidate 21 days later. Blood was collected and serum was isolated from 5 randomly selected control mice at the start of the study and from all mice on study day 21 and 34. Serum was stored at −80° C. until analysis for antibody titers. On study day 34, mice were euthanized and spleens were harvested.

Design and preparation of mRNA. mRNA encoding the SARS-COV-2 full length spike protein and flanked with the same UTRs used in the BNT162b2 (Comirnaty) vaccine was purchased from Vernal Biosciences. All uridine nucleosides were substituted with N1-methyl-pseudouridine. To produce the mRNA, a synthetic gene encoding the mRNA sequence (VRN029; SEQ ID NO: 211) was cloned into a DNA plasmid:

GGGAATAAACTAGTATTCTTCTGGTCCCCACAGACTCAGAGAGAACCCGCCACCATGTTCGTGT
TCCTGGTGCTGCTGCCTCTGGTGTCCAGCCAGTGTGTGAACCTGACCACCAGAACACAGCTGCC
TCCAGCCTACACCAACAGCTTTACCAGAGGCGTGTACTACCCCGACAAGGTGTTCAGATCCAGC
GTGCTGCACTCTACCCAGGACCTGTTCCTGCCTTTCTTCAGCAACGTGACCTGGTTCCACGCCA
TCCACGTGTCCGGCACCAATGGCACCAAGAGATTCGACAACCCCGTGCTGCCCTTCAACGACGG
GGTGTACTTTGCCAGCACCGAGAAGTCCAACATCATCAGAGGCTGGATCTTCGGCACCACACTG
GACAGCAAGACCCAGAGCCTGCTGATCGTGAACAACGCCACCAACGTGGTCATCAAAGTGTGCG
AGTTCCAGTTCTGCAACGACCCCTTCCTGGGCGTCTACTACCACAAGAACAACAAGAGCTGGAT
GGAAAGCGAGTTCCGGGTGTACAGCAGCGCCAACAACTGCACCTTCGAGTACGTGTCCCAGCCT
TTCCTGATGGACCTGGAAGGCAAGCAGGGCAACTTCAAGAACCTGCGCGAGTTCGTGTTTAAGA
ACATCGACGGCTACTTCAAGATCTACAGCAAGCACACCCCTATCAACCTCGTGCGGGATCTGCC
TCAGGGCTTCTCTGCTCTGGAACCCCTGGTGGATCTGCCCATCGGCATCAACATCACCCGGTTT
CAGACACTGCTGGCCCTGCACAGAAGCTACCTGACACCTGGCGATAGCAGCAGCGGATGGACAG
CTGGTGCCGCCGCTTACTATGTGGGCTACCTGCAGCCTAGAACCTTCCTGCTGAAGTACAACGA
GAACGGCACCATCACCGACGCCGTGGATTGTGCTCTGGATCCTCTGAGCGAGACAAAGTGCACC
CTGAAGTCCTTCACCGTGGAAAAGGGCATCTACCAGACCAGCAACTTCCGGGTGCAGCCCACCG
AATCCATCGTGCGGTTCCCCAATATCACCAATCTGTGCCCCTTCGGCGAGGTGTTCAATGCCAC
CAGATTCGCCTCTGTGTACGCCTGGAACCGGAAGCGGATCAGCAATTGCGTGGCCGACTACTCC
GTGCTGTACAACTCCGCCAGCTTCAGCACCTTCAAGTGCTACGGCGTGTCCCCTACCAAGCTGA
ACGACCTGTGCTTCACAAACGTGTACGCCGACAGCTTCGTGATCCGGGGAGATGAAGTGCGGCA
GATTGCCCCTGGACAGACAGGCAAGATCGCCGACTACAACTACAAGCTGCCCGACGACTTCACC
GGCTGTGTGATTGCCTGGAACAGCAACAACCTGGACTCCAAAGTCGGCGGCAACTACAATTACC
TGTACCGGCTGTTCCGGAAGTCCAATCTGAAGCCCTTCGAGCGGGACATCTCCACCGAGATCTA
TCAGGCCGGCAGCACCCCTTGTAACGGCGTGGAAGGCTTCAACTGCTACTTCCCACTGCAGTCC
TACGGCTTTCAGCCCACAAATGGCGTGGGCTATCAGCCCTACAGAGTGGTGGTGCTGAGCTTCG
AACTGCTGCATGCCCCTGCCACAGTGTGCGGCCCTAAGAAAAGCACCAATCTCGTGAAGAACAA
ATGCGTGAACTTCAACTTCAACGGCCTGACCGGCACCGGCGTGCTGACAGAGAGCAACAAGAAG
TTCCTGCCATTCCAGCAGTTTGGCCGGGATATCGCCGATACCACAGACGCCGTTAGAGATCCCC
AGACACTGGAAATCCTGGACATCACCCCTTGCAGCTTCGGCGGAGTGTCTGTGATCACCCCTGG
CACCAACACCAGCAATCAGGTGGCAGTGCTGTACCAGGACGTGAACTGTACCGAAGTGCCCGTG
GCCATTCACGCCGATCAGCTGACACCTACATGGCGGGTGTACTCCACCGGCAGCAATGTGTTTC
AGACCAGAGCCGGCTGTCTGATCGGAGCCGAGCACGTGAACAATAGCTACGAGTGCGACATCCC
CATCGGCGCTGGAATCTGCGCCAGCTACCAGACACAGACAAACAGCCCTCGGAGAGCCAGAAGC
GTGGCCAGCCAGAGCATCATTGCCTACACAATGTCTCTGGGCGCCGAGAACAGCGTGGCCTACT
CCAACAACTCTATCGCTATCCCCACCAACTTCACCATCAGCGTGACCACAGAGATCCTGCCTGT
GTCCATGACCAAGACCAGCGTGGACTGCACCATGTACATCTGCGGCGATTCCACCGAGTGCTCC
AACCTGCTGCTGCAGTACGGCAGCTTCTGCACCCAGCTGAATAGAGCCCTGACAGGGATCGCCG
TGGAACAGGACAAGAACACCCAAGAGGTGTTCGCCCAAGTGAAGCAGATCTACAAGACCCCTCC
TATCAAGGACTTCGGCGGCTTCAATTTCAGCCAGATTCTGCCCGATCCTAGCAAGCCCAGCAAG
CGGAGCTTCATCGAGGACCTGCTGTTCAACAAAGTGACACTGGCCGACGCCGGCTTCATCAAGC
AGTATGGCGATTGTCTGGGCGACATTGCCGCCAGGGATCTGATTTGCGCCCAGAAGTTTAACGG
ACTGACAGTGCTGCCTCCTCTGCTGACCGATGAGATGATCGCCCAGTACACATCTGCCCTGCTG
GCCGGCACAATCACAAGCGGCTGGACATTTGGAGCAGGCGCCGCTCTGCAGATCCCCTTTGCTA
TGCAGATGGCCTACCGGTTCAACGGCATCGGAGTGACCCAGAATGTGCTGTACGAGAACCAGAA
GCTGATCGCCAACCAGTTCAACAGCGCCATCGGCAAGATCCAGGACAGCCTGAGCAGCACAGCA
AGCGCCCTGGGAAAGCTGCAGGACGTGGTCAACCAGAATGCCCAGGCACTGAACACCCTGGTCA
AGCAGCTGTCCTCCAACTTCGGCGCCATCAGCTCTGTGCTGAACGATATCCTGAGCAGACTGGA
CCCTCCTGAGGCCGAGGTGCAGATCGACAGACTGATCACAGGCAGACTGCAGAGCCTCCAGACA
TACGTGACCCAGCAGCTGATCAGAGCCGCCGAGATTAGAGCCTCTGCCAATCTGGCCGCCACCA
AGATGTCTGAGTGTGTGCTGGGCCAGAGCAAGAGAGTGGACTTTTGCGGCAAGGGCTACCACCT
GATGAGCTTCCCTCAGTCTGCCCCTCACGGCGTGGTGTTTCTGCACGTGACATATGTGCCCGCT
CAAGAGAAGAATTTCACCACCGCTCCAGCCATCTGCCACGACGGCAAAGCCCACTTTCCTAGAG
AAGGCGTGTTCGTGTCCAACGGCACCCATTGGTTCGTGACACAGCGGAACTTCTACGAGCCCCA
GATCATCACCACCGACAACACCTTCGTGTCTGGCAACTGCGACGTCGTGATCGGCATTGTGAAC
AATACCGTGTACGACCCTCTGCAGCCCGAGCTGGACAGCTTCAAAGAGGAACTGGACAAGTACT
TTAAGAACCACACAAGCCCCGACGTGGACCTGGGCGATATCAGCGGAATCAATGCCAGCGTCGT
GAACATCCAGAAAGAGATCGACCGGCTGAACGAGGTGGCCAAGAATCTGAACGAGAGCCTGATC
GACCTGCAAGAACTGGGGAAGTACGAGCAGTACATCAAGTGGCCCTGGTACATCTGGCTGGGCT
TTATCGCCGGACTGATTGCCATCGTGATGGTCACAATCATGCTGTGTTGCATGACCAGCTGCTG
TAGCTGCCTGAAGGGCTGTTGTAGCTGTGGCAGCTGCTGCAAGTTCGACGAGGACGATTCTGAG
CCCGTGCTGAAGGGCGTGAAACTGCACTACACATGATGACTCGAGCTGGTACTGCATGCACGCA
ATGCTAGCTGCCCCTTTCCCGTCCTGGGTACCCCGAGTCTCCCCCGACCTCGGGTCCCAGGTAT
GCTCCCACCTCCACCTGCCCCACTCACCACCTCTGCTAGTTCCAGACACCTCCCAAGCACGCAG
CAATGCAGCTCAAAACGCTTAGCCTAGCCACACCCCCACGGGAAACAGCAGTGATTAACCTTTA
GCAATAAACGAAAGTTTAACTAAGCTATACTAACCCCAGGGTTGGTCAATTTCGTGCCAGCCAC
ACCCTGGAGCTAGCAGCGGCCGCGGCCGCAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAA

The synthetic gene was comprised of an RNA promoter, a 5′ untranslated region, the SARS-COV2 Spike protein receptor binding domain, a 3′ untranslated region, and a poly(A) tail region of approximately 120 As. The plasmid was propagated and expanded in a culture of E. coli and then isolated from the clarified E. coli lysate via anion exchange chromatography. The purified plasmid was linearized using a type IIs restriction enzyme that cut at a site at the end of the poly (A) tail encoding region. That plasmid was then incubated in a buffer with nucleotide triphosphates, RNA polymerase, and RNase inhibitor. To stop the reaction, DNase I was added to digest the linear plasmid template. The uncapped RNA was then purified using chromatography and then incubated in another buffer with GTP, S-adenosylmethionine, a guanalyltransferase, 2′-O-methyltransferase, and RNase inhibitor. The capped mRNA was then purified using chromatography, buffer exchanged into water, and filled into vials.

Generation of lipid nanoparticles (LNP) containing mRNA. Stock solutions of each lipid were prepared. Ionizable lipids were weighed out in 4 mL glass vials (Thermo B7999-2) and dissolved in ethanol (Sigma-Aldrich 200 proof, RNase free) to a final concentration of 10 mM. Other lipids such as DSPC (Avanti Polar Lipids), Cholesterol (Dishman) and PEG-DMG (NOF) were weighed out and dissolved in ethanol to a concentration of 1 mM. DSPS-Na (NOF) was dissolved in methanol (Sulpelco, Omnisolve) at a concentration of 1 mM and briefly heated to 70° C. to complete its dissolution.

Lipid mixtures for each individual LNP were prepared by adding the desired volume of each lipid stock solution to a new vial, adding ethanol if needed to achieve a final volume of 1.2 mL. For example, a LNP formulation of AKG-UO-1/DSPC/DSPS/Chol/PEG-DMG (50/2.5/7.5/38.5/1.5 mol %), with an N/P of 5 contained 1500 nmol AKG-UO-1, 75 nmol DSPC, 225 nmol DSPS, 1155 nmol Chol and 45 nmol PEG-DMG for every 100 μg of mRNA used. mRNA solutions were prepared by thawing frozen mRNA (SARS-COV-2 spike mRNA, Vernal) vials and diluting mRNA in 6.25 mM sodium acetate (pH 5.0) to a final concentration of 0.033 mg/mL, where the concentration is confirmed by absorbance on a Nanodrop.

To prepare LNPs, a NanoAssemblr Benchtop microfluidic device (from Precision Nanosystems) was used. If LNPs contained DSPS, the heating block accessory set to 70° C. was used, otherwise LNPs were mixed at room temperature. 3 mL of mRNA solution was loaded into a 3 mL disposable syringe (BD 309656) and 1 ml of lipid mixture in a 1 ml syringe (BD309659) and placed in the NanoAssemblr heating block for 4 min prior to mixing. LNP formation was achieved by pumping the liquid streams through a disposable microfluidics cassette at 3:1 aqueous:alcohol volume ratio at 6 mL/min mixing speed. After mixing, 3.6 mL of LNP mixture was collected, while the initial mixed volume of 0.35 mL and last 0.05 mL of mix was discarded. Ethanol was removed by buffer exchange using SpectraPor dialysis tubing (12-14k MWCO) in PBS (Cytivia, SH30256.01). LNPs were typically exchanged into PBS, pH 7.4 and then 15 mM Tris, pH 7.4, 20% sucrose, concentrated to 20-50 μg/mL mRNA, sterile filtered (Thermo Nalgene 0.2 um #720-1320) prior to freezing by immersion in liquid nitrogen for 5 min and long-term storage at −20° C. For this study, samples were concentrated to >40 μg/mL mRNA, and diluted with varying volumes of 15 mM Tris, 20% Sucrose, pH 7.4 to a target concentration of 40 μg mRNA and then frozen on LN2. Characterization of LNPs was undertaken after an aliquot of the LNPs were thawed and diluted 1:1 (vol:vol) with 15 mM Tris, pH 7.4 such that the final concentration was 20 μg/mL mRNA in 15 mM Tris, 10% sucrose, pH 7.4. This simulated the conditions of sample preparation that were performed prior to dosing the animals with an injection of 1 μg mRNA in 50 μL volume via IM injection into a hind limb.

LNP Characterization. mRNA encapsulation and mRNA concentration within the LNPs was measured using a Ribogreen assay. Nanoparticle size and zeta potential were measured by a zetasizer (Malvern).

SARS-COV-2 anti-spike antibody titers. A standard indirect ELISA was performed to analyze serum samples for total IgG binding antibodies to the SARS-COV-2 spike protein. For this assay, Nunc MaxiSorp 96-well plates were coated with 100 μL of SARS-COV-2 spike protein (Sino Biological, cat. no. 40589-V08B1) diluted to 2 μg/mL in 1×PBS, pH 7.4. Plates were incubated statically for 12 hrs at 37° C. Unbound coating antigen was removed by washing plates 3× with 100 μL PBS+0.05% Tween-20. Plates were then blocked in PBS+5% skim milk for 1 hr at 37° C. Test and positive control samples were diluted in assay diluent (PBS, Tween-20, 1% skim milk) to starting point dilution 1:20 followed by four-fold serial dilutions using U-bottom dilution plates. Once blocking was completed, blocking buffer was removed by inversion and each sample was plated in duplicates. Plates were statically incubated for 2 hr at 37° C., followed by washing 3× with 100 μL of PBS+0.05% Tween-20 to remove unbound sera. 100 μL of secondary detection antibody (goat anti-mouse-HRP IgG, Abcam) was added to each well at a dilution of 1:10,000. Plates were incubated statically for 30 min at RT, and unbound antibodies were subsequently removed and plates were washed as described above. To develop, 100 μL of 1-Step Ultra TMB substrate was added to each well and the reaction was stopped after ~10 min with 50 μL of TMB stop solution (SeraCare, cat. no. 5150-0019). The plates were read within 30 min at 450 nm with a Thermo LabSystems Multiskan spectrophotometer. Titers were defined as the reciprocal of the dilutions that generated a specific cut-off value for OD 450 on the linear part of the titration curve.

Ex vivo T cell responses. On study day 34, spleens were mechanically dissociated to single-cell suspensions. Cells were resuspended in cell-stimulation media (RPMI with L-Glutamine and HEPES buffer, heat-inactivated fetal bovine serum, and Pen/Strep) and 2×10⁶ cells were aliquoted in a volume of 100 μL into 96-well plates. Splenocytes from each mouse were stimulated for approximately 18 hrs at 37° C. with 100 μL of media alone, treated with positive control Cell Stimulation Cocktail (ThermoFisher, cat. #00-4970-93) containing PMA and ionomycin, or 1 μg/mL of a peptide pool covering the SARS-COV-2 spike protein (JPT, cat. #PM-WCPV-S-2). After 1 hr of stimulation, Golgi Stop (BD Biosciences, cat. #554724) was added to each well.

Flow cytometry. After the stimulation, cells were washed with PBS and transferred to 96-well deep-well plates. Cells were stained with LIVE/DEAD near IR viability dye (ThermoFisher, cat. #L10119) diluted in PBS for 20 min at 4° C. Cells were washed with FBS staining buffer (BD Biosciences, cat. #554656) and incubated with Fc Block (BD Biosciences, cat. 553142) for 10 min at 4° C. Cells were then stained for 40 min at 4° C. with a cell surface antibody cocktail consisting of CD3 BV605 (BD, cat. #564009), CD4 BV421 (BD, cat. #562891), and CD8 APC (BD, cat. #553035). BD Brilliant Stain Buffer (cat. #563794) was included in the staining buffer. Cells were washed with FBS staining buffer and then fixed for 20 min at room temperature with Fix/Perm buffer (ThermoFisher, cat. #00-5523-00). Cells were washed with 1× Perm buffer, incubated with Fc Block, and then stained for 40 min at 4° C. with an intracellular cytokine antibody cocktail consisting of IFN-g PE/Cy7 (BD, cat. #557649), IL-2 PE (BioLegend, cat. #503808) and TNF-a FITC (BD, cat. #554418). Cells were then washed, resuspended in FBS stain buffer and acquired on a MACSQuant 16 flow cytometer (Miltenyi Biotec). Flow data was analyzed using FlowJo v10.8.1 (BD Biosciences).

TABLE 11
Physicochemical properties of LNPs used in evaluating the immunogenicity
of SARS-CoV-2 spike protein mRNA vaccine construct

| | Particle | Zeta | Zeta | |
|---|---|---|---|---|
| | Size | Potential | Potential | Encapsulation |
| LNP Formulation | (nm) | (mV) pH 5 | (mV) pH 7 | Efficiency (%) |
| 7.5% DSPS_U01_PEG-DMG 1.5% | 80.3 | 5.3 | −2.6 | 90.7 ± 7.1 |
| 7.5% DSPS_UO1_PEG-DMG 2.5% | 71.0 | 5.5 | −1.0 | 89.1 ± 9.3 |
| 7.5% DSPS_UO1_PEG-DMG 3.5% | 61.9 | 6.6 | −3.6 | 89.3 ± 10.0 |
| 7.5% DSPS_UO1_PEG-DPPE 1.5% | 80.7 | 7.9 | −5.1 | 90.7 ± 14.2 |
| 7.5% DSPS_UO1_PEG-DPPE 2.5% | 68.4 | 2.7 | −2.4 | 91.0 ± 6.9 |
| 7.5% DSPS_UO1_PEG-DSG 1.5% | 100.7 | 9.6 | −6.7 | 88.8 ± 5.1 |
| 7.5% DPPS_UO1_PEG-DMG 1.5% | 89.8 | 14.4 | −1.4 | 93.8 ± 9.2 |
| UO1_PEG-DMG 1.5% | 116.0 | 20.2 | 2.5 | 86.5 ± 9.3 |
| 7.5% DSPS_KC2OA_PEG-DMG 1.5% | 80.0 | 11.7 | −3.5 | 90.9 ± 5.5 |
| 7.5% DSPS_KC2OA_PEG-DSG 1.5% | 88.5 | 9.0 | −3.9 | 92.2 ± 9.7 |
| ALC0315_1.5% PEG-DMG | 79.5 | 2.0 | −2.4 | 86.7 ± 9.3 |
| SM102_1.5% PEG-DMG | 92.6 | 11.8 | −0.1 | 92.0 ± 7.1 |

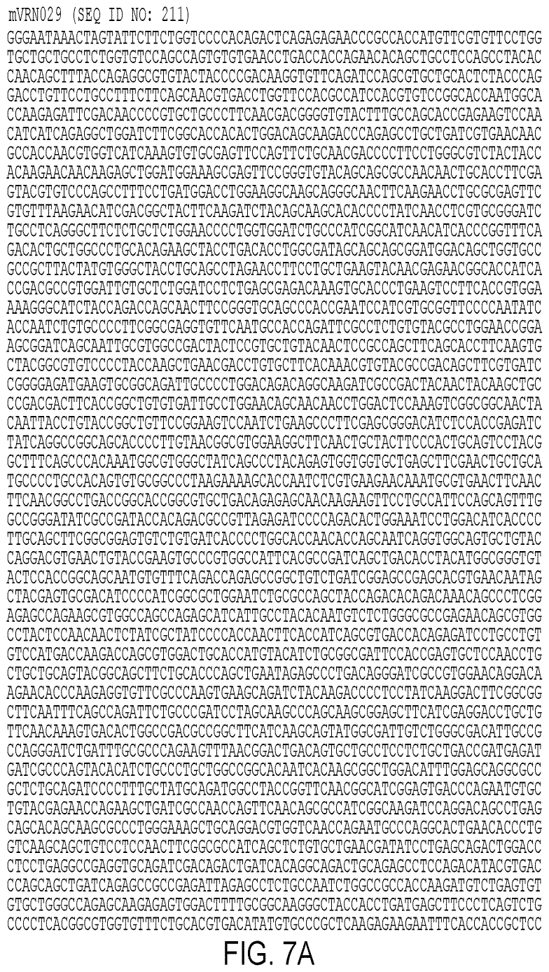

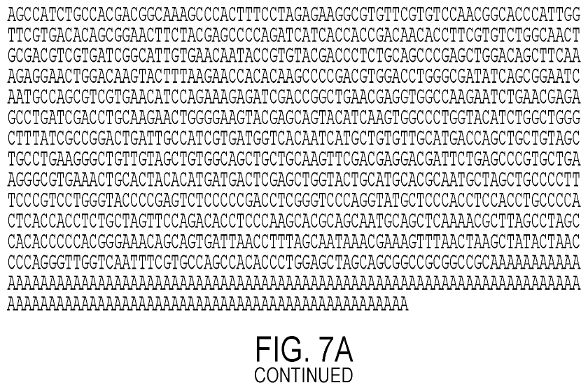

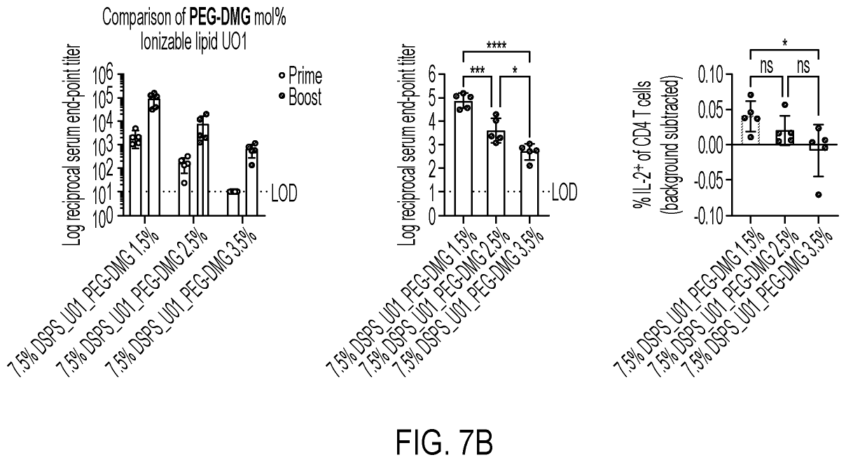

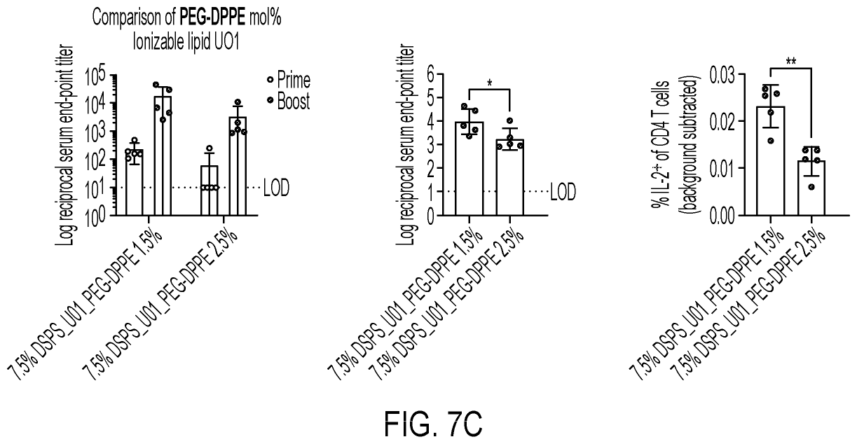

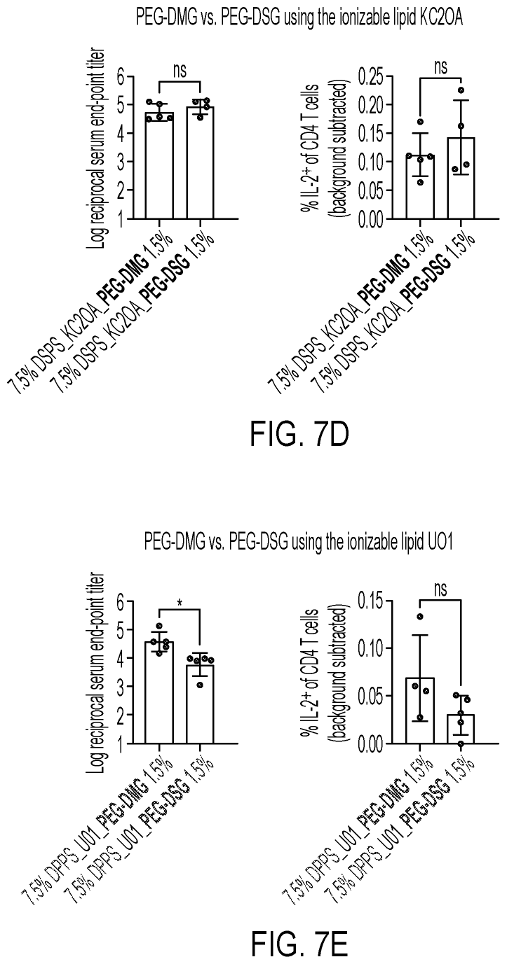

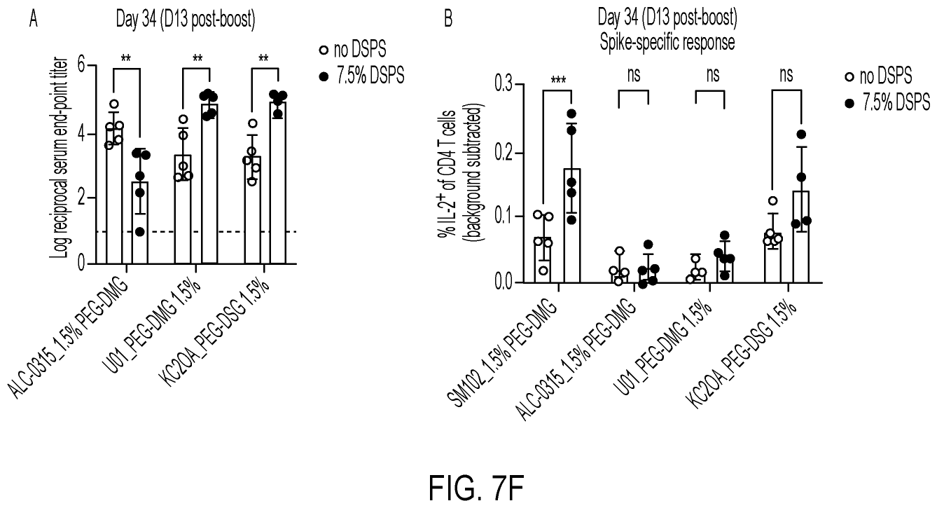

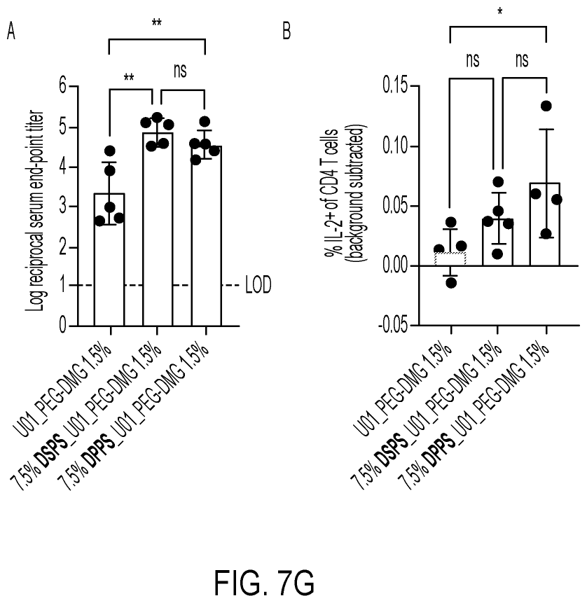

To evaluate the impact of PEG-lipid concentration on vaccine immunogenicity, BALB/c mice were immunized with mRNA-LNPs containing increasing amounts of PEG-lipid (PEG-DMG or PEG-DPPE). Blood serum was collected on day 21 post prime and on day 13 post boost (day 34 of study) for antibody analysis. Splenocytes were stimulated with Spike peptide pools and the percent of CD4 T cells producing IL-2 was quantified using flow cytometry (FIG. 7A and FIG. 7B). Both forms of PEG, PEG-DMG (FIG. 7A) or PEG-DPPE (FIG. 7B), inversely impacted mRNA-LNPA vaccine immunogenicity, with lower concentrations of PEG-lipid showing higher levels of both B-cell and T-cell responses.

To evaluate the impact of PEG-lipid acyl chain composition on mRNA-LNP immunogenicity, BALB/c mice were immunized with mRNA-LNPs containing different PEG formats (PEG-DMG or PEG-DSG). Blood serum was collected on day 21 post prime and on day 13 post boost (day 34 of study). Splenocytes were stimulated with Spike peptide pools and the percent of CD4 T cells producing IL-2 was quantified using flow cytometry. Antibody titer data were log-transformed prior to statistical analysis. Groups were compared using an unpaired t test. For LNPs made with the ionizable lipid KC20A, either PEG format performed similarly (FIG. 7C). In contrast, LNPs using the ionizable lipid UO1 and PEG-DMG induced a superior antibody response than LNPs containing PEG-DSG (FIG. 7D).

To assess how the incorporation of phosphatidylserine influences mRNA-LNP immunogenicity, BALB/c mice were immunized with mRNA-LNPs containing various ionizable lipids with or without DSPS. On Day 34 (13 days post-boost), serum was collected for quantification of total IgG anti-spike antibodies by ELISA. Splenocytes were stimulated with peptide and the percent of CD4 T cells producing IL-2 was quantified using flow cytometry.

The inclusion of DSPS with comparator ionizable lipid ALC-0315 negatively impacted total IgG anti-spike antibody levels (FIG. 7E). DSPS had the opposite effect on LNPs containing the ionizable lipids UO1 and KC20A and significantly increased geometric mean antibody levels 36- and 46-fold, respectively. DSPS also had an effect on CD4 T cell responses, with responses trending higher for LNPs containing UO1 and KC20A, and significantly higher for SM-102. Taken together, inclusion of DSPS in lipid nanoparticles can substantially influence the immunogenicity of mRNA vaccines, and that the impact of DSPS is influenced by the type of ionizable cationic lipid.

The effects of different phosphatidylserine acyl chain composition on serum antibody titers (FIG. 7F, panel A) and the magnitude of the spike-specific CD4 T cell response in the spleen (FIG. 7F, panel B) was evaluated. Mice were immunized with LNPs using the ionizable lipid UO1 and either the DSPS or DPPS forms of phosphatidyl serine. On Day 34 (13 days post-boost), serum was collected for quantification of total IgG anti-spike antibodies by ELISA. Splenocytes were stimulated with peptide and the percent of CD4 T cells producing IL-2 was quantified using flow cytometry.

With regards to the impact of the PS acyl chain composition, both forms of PS comparably increased antibody levels over the base formulation lacking PS (FIG. 7G, Panel A). Both forms of PS also had a positive effect on the CD4 T cell response (FIG. 7G, Panel B), although only the formulation containing DPPS was significantly higher than the based formulation without PS. Taken together, both forms of PS included in our LNPs, DPPS or DSPS, had a similar positive effect on increasing the immunogenicity of mRNA-LNP vaccines.

### Example 14. Measuring the Effect of Adding Either DSPS D-Isomer or L-Isomer at 7.5 Mol % in KC2-01 Based LNPs by Particle Characteristics and Activity in MutuDC1940 Dendritic Cell Line

The aim of this study was to compare the impact of including the D and the L-isomers of DSPS into mRNA LNP formulations at 7.5 mol %. LNPs were prepared as described in Example 2 at 25° C. and analyzed as in Example 3. The LNPs contained KC2-01 as the ICL at an N/P ratio of 5 with 2.5 mol % DSPC, 7.5 mol % DSPS (D or L) and a constant 1.5 mol % PEG-DMG. Transfection efficiency was evaluated in murine dendritic cells as described in Example 4.

TABLE 12
Physicochemical properties of KC2-01 LNPs with mCherry mRNA and distearoylphosphatidyl-
L-serine or distearoylphosphatidyl-D-serine.

| | DSPS | | | Zeta | Zeta | |
|---|---|---|---|---|---|---|
| | (serine | Particle | | Potential | Potential | Encapsulation |
| LNP Formulation | isomer) | Size (nm) | PDI | mV, pH 5 | mV, pH 7 | Efficiency |
| KC2-01/DSPC/Chol/PEG-DMG (50/10/38.5/1.5) | N/A | 83.92 | 0.03 | 7.7 | −0.47 | 89.2 ± 5.7 |
| KC2-01/DSPC/DSPS/Chol/PEG-DMG (50/2.5/7.5/38.5/1.5) | D | 85.4 | 0.09 | 7.8 | −4.8 | 88.7 ± 3.5 |
| KC2-01/DSPC/DSPS/Chol/PEG-DMG (50/2.5/7.5/38.5/1.5) | L | 87.4 | 0.04 | 7.7 | −2.7 | 90.9 ± 6.0 |

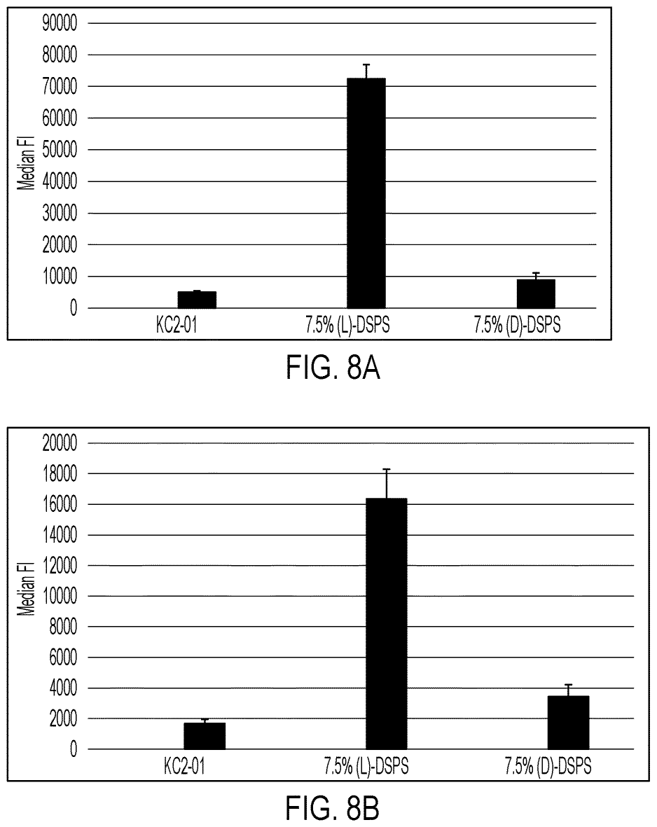

This study shows that including 7.5 mol % DSPS (either isomer) in a KC2-01 based LNP formulation had no effect on the particle size or mRNA encapsulation. The zeta potential values are similar at pH 5, but the DSPS containing formulation have more negative values at pH 7 than the non-DSPS containing formulation, likely a result of the additional negative charge added by the DSPS. The impact of the stereochemistry on transfection was evaluated in FIG. 8A and FIG. 8B. An 8-fold increase in mCherry expression was observed when the D-isomer of DSPS was used compared to the L isomer at 1 μg/mL mRNA (and 4.7-fold at 0.33 μg/mL mRNA), indicating that the uptake or expression mechanism(s) of DSPS containing LNPs is likely stereospecific.

### Example 13. Measuring the Effect of Inclusion of DSPS in KC2-Based LNPs at 25° C. And 65° C. On Particle Characteristics and Activity in MutuDC1940 Dendritic Cell Line

The aim of this study was to compare the impact of including the D-isomer of DSPS in KC2 based LNPs produced at two different temperatures. LNPs were prepared as described in Example 2 where those that contained DSPS were made at 65° C. at those that did not include DSPS were made at 25° C. and all LNPs were analyzed as in Example 3.

All LNPs contained ICL at an N/P ratio of 5 with a constant 1.5 mol % PEG-DMG. The cholesterol content was held constant at 38.5 mol % and the DSPC content varied inversely with the mol % of DSPS at 5 and 7.5 mol % (all lipid concentrations were used as mol % of total lipid). These mRNA LNPs all contained mCherry mRNA and the composition of comparator formulations using SM-102 based lipid formulation, the same lipid composition as that used in mRNA-1273 and that using ALC-0315, similar to that used in BNT162b2 were taken from their respective prescribing information. Note, the BNT162b2 comparator using ALC-0315 was made with an N/P of 5.0 in keeping with the other formulations in this study, which is different than the approved Covid vaccine Comirnaty.

TABLE 13
Physicochemical properties of KC2 containing LNPs with 5 or 7.5% DSPS to
similar LNPs prepared with SM102 or ALC-0315 ionizable cationic lipid.

| | | | | | Zeta Potential | Encapsulation |
|---|---|---|---|---|---|---|
| | Mixing | Particle Size | | Zeta Potential | mV, pH | Efficiency |
| LNP Formulation | Temp ° C. | (nm) | PDI | mV, pH 5 | 7 | (%) |
| KC2 | 25 | 70.4 | 0.21 | 4.5 | −1.1 | 87.3 ± 3.2 |
| KC2 with 5% DSPS | 25 | 78.3 | 0.12 | 2.5 | −2.2 | 87.0 ± 2.2 |
| KC2 with 5% DSPS | 65 | 88.2 | 0.11 | 7.4 | −4.2 | 87.0 ± 3.8 |
| KC2 with 7.5% DSPS | 25 | 73.5 | 0.11 | 1.0 | −2.3 | 86.9 ± 3.8 |
| KC2 with 7.5% DSPS | 65 | 74.3 | 0.07 | 0.5 | −8.2 | 87.8 ± 2.2 |
| SM102 | 25 | 84.0 | 0.17 | 6.3 | −0.7 | 81.9 ± 3.9 |
| ALC-0315 | 25 | 72.1 | 0.08 | 3.3 | −3.0 | 80.7 ± 2.2 |

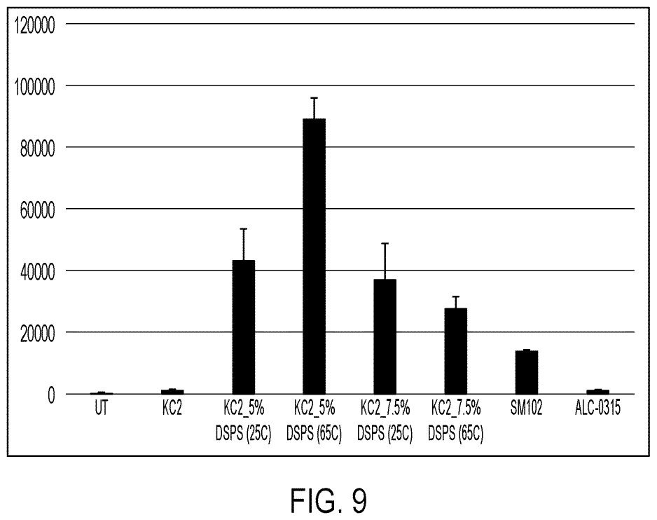

This study shows that heating the lipid solution and mRNA solution in a heating block set to 65° C. had no deleterious effect on particle diameter or, zeta potential or mRNA encapsulation readings at either 5 or 7.5 mol % DSPS content. The impact of temperature on transfection of DSPS-targeted KC2 LNPs, and the comparison to SM102 and ALC-0315 containing LNPs is shown in FIG. 9. The 5 mol % DSPS formulation had a 2-fold higher mCherry expression than the exact same formulation made at 25° C. (p<0.05, T-Test). A comparison of the formulations with 7.5 mol % DSPS made at either temperature showed no significant difference. The 5 mol % DSPS containing LNP, made at 65° C. gave a 6.5-fold increase in mCherry expression over the SM102 formulation. It is likely that higher temperatures increase the solubility of DSPS in alcohol and allow better incorporation of the lipid in the particle, which allows for enhanced uptake in this in vitro study, while nor adversely affecting many of the LNP crucial particle characteristics such as size, encapsulation or mRNA expression.

### Example 14. Evaluating the Effect of Adding DSPS to UO1, SM102 and ALC-0315 Based LNPs by Comparing Particle Characteristics and Activity in MutuDC1940 Dendritic Cell Line

The aim of this study was to evaluate the effect of including the L-isomers of DSPC into mRNA LNP formulations at 0, 5% and 7.5 mol %. LNPs were prepared as described in Example 2 at 25° C. and analyzed as in Example 3.

The LNPs contained either AKG-UO-1 (“UO-1”), SM102 as the ICL at 50 mol %. For UO1 and SM102 the formulations contained 38.5 mol % cholesterol, and 1.5 mol % PEG-DMG. The non DSPS containing samples have 10 mol % DSPC, and to the DSPS containing LNPs, DSPS was added at 5 or 7.5 mol % with a concomitant reduction in DSPC by the same mol %. For the ALC-0315 formulation, the ICL was 46.3 mol %, DSPC 9.4 mol %, cholesterol 42.7 mol % and PEG-DMG 1.5 mol %. DSPS was added with concomitant reduction in the DSPC mol % as above. The N/P in all formulations was 5. Transfection efficiency was evaluated in murine dendritic cells as described in Example 4.

TABLE 14
Physicochemical properties and encapsulation efficiency of PS-
targeted formulations of UO1, SM102, and ALC-0315 containing LNPs

| | | | Zeta | Zeta | Encapsulation |
|---|---|---|---|---|---|
| | Particle | | Potential | Potential | Efficiency |
| LNP Formulation | Size (nm) | PDI | mV, pH 5 | mV, pH 7 | (%) |
| UO1 | 72.8 | 0.11 | 10.2 | 3.6 | 89.6 ± 4.8 |
| UO1_5%_DSPS | 83.0 | 0.11 | 9.9 | −2.7 | 91.4 ± 4.2 |
| UO1_7.5%_DSPS | 80.5 | 0.03 | 11.0 | 1.4 | 91.1 ± 6.3 |
| SM102 | 80.7 | 0.04 | 13.0 | 1.6 | 90.1 ± 5.6 |
| SM102_5%_DSPS | 81.0 | 0.02 | 13.1 | −6.1 | 90.5 ± 1.4 |
| SM102_7.5%_DSPS | 80.1 | 0.05 | 13.0 | −4.0 | 90.1 ± 2.7 |
| ALC0315 | 74.6 | 0.06 | 6.2 | −0.9 | 72.3 ± 0.9 |
| ALC0315_5%_DSPS | 68.1 | 0.10 | 8.0 | −8.8 | 84.2 ± 1.3 |
| ALC0315_7.5%_DSPS | 75.0 | 0.02 | 7.9 | −11.8 | 84.9 ± 3.8 |

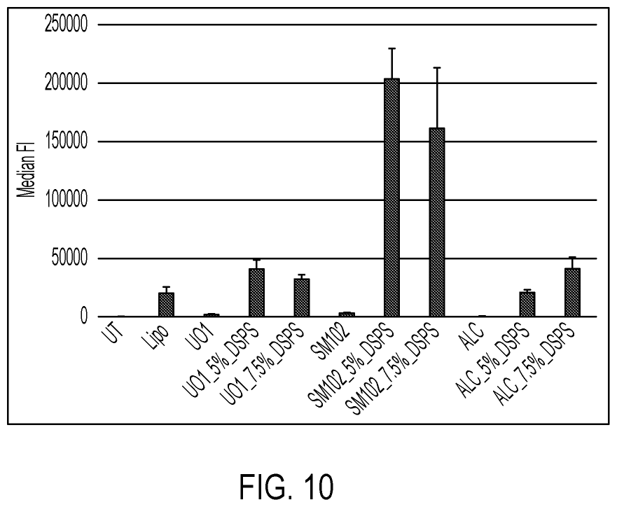

This study shows that including DSPS in LNPs based on UO1, SM102 and ALC-0315 caused an increase in expression of mCherry (FIG. 10), without adverse changes in particle size or mRNA entrapment. For UO-1 and SM102, 5 mol % DSPS looked maximal, while 7.5 mol % DSPS was maximal for the ALC-0315 formulation. For UO-1 and SM102 the increase in expression was 18.1 and 58.7-fold respectively for the 5 mol % DSPS samples. The increase was 80.9-fold for the ALC-0315 based sample for 7.5 mol % DSPS.

### Example 15. Additional Exemplary Phosphatidyl-L-Serine Targeted LNP Formulations

Additional formulations shown below could also be prepared using the methods described in the Examples above. All lipid concentrations are shown as mol % as a percentage of the total lipid in the LNP. The below compositions vary in conjugated lipid content from 0.5 to 2.5 mol %, in sterol content from 25-45 mol %, in ICL content from 40-65 mol %, in saturated phosphatidyl-L-serine content from 2-10 mol %, and in total noncationic phospholipid content from 5-20 mol %. Most compositions would contain two phospholipids, typically phosphatidyl-L-serine (DSPS or DPPS being preferred) and phosphatidylcholine, although some exemplary formulations may contain more than two phospholipids, including phosphatidylethanolamines, like dioleoylphosphatidylethanolamine (DOPE).

TABLE 15
Exemplary phosphatidyl-L-serine containing LNP formulations

| | Ionizable | | Additional | | Conjugated |
|---|---|---|---|---|---|
| Formulation | Cationic Lipid | PS (mol | Phospholipid(s) | Sterol | lipid |
| (#) | (mol %) | %) | (mol %) | (mol %) | (mol %) |
| 1 | KC3-0A (47.5) | DSPS (7.5) | DSPC (3.5) | Chol (40) | PEG-DMG (1.5) |
| 2 | KC3-0A (45) | DSPS (8) | DSPC (4) | Chol (42) | PEG-DMG (1) |
| 3 | AKG-KC2-01 (40) | DSPS (5) | DSPC (5) | Chol (44) | PEG-DMG (1) |
| | | | DOPE (5) | | |
| 4 | KC3-PA (42.5) | DSPS (7.5) | DSPC (6.5) | Chol (42) | PEG-DMG (1.5) |
| 5 | AKG-KC3-01 (65) | DSPS (7.5) | DSPC (2.5) | Chol (24.5) | PEG-DMG (0.5) |
| 6 | AKG-KC32-01 (60) | DSPS (6) | DSPC (4) | Chol (29) | PEG-DMG (1) |
| 7 | KC3-PA (55) | DSPS (7) | DSPC (3) | Chol (34) | PEG-DMG (1) |
| 8 | AKG-KC2-01 (57) | DSPS (6.5) | DSPC (3.5) | Chol (28) | PEG-DMG (1) |
| 9 | AKG-KC2-01 (60) | DSPS (7.5) | DSPC (2.5) | Chol (28.5) | PEG-DMG (1.5) |
| | | | DOPE (4) | | |
| 10 | AKG-KC2-01 (48) | DSPS (6) | DSPC (4) | Chol (41.5) | PEG-DMG (0.5) |
| 11 | KC3-PA (48) | DPPS (6) | DSPC (4) | Chol (41.5) | PEG-DMG (0.5) |
| 12 | AKG-KC2-01 (48) | DSPS (6) | DPPC (4) | Chol (41.5) | PEG-DMG (0.5) |
| 13 | AKG-KC2-PA (47.5) | DSPS (6) | DSPC (4) | Chol (41.5) | PEG-DMG (1) |
| 14 | KC3-PA (47.5) | DPPS (6) | DSPC (4) | Chol (41.5) | PEG-DMG (1) |
| 15 | AKG-KC2-PA(47.5) | DSPS (6) | DPPC (4) | Chol (41.5) | PEG-DMG (1) |
| 16 | KC3-0A (48) | DSPS (7.5) | DSPC (2.5) | Chol (41.5) | PEG-DMG (0.5) |
| 17 | KC3-C17(8:1) (48) | DPPS (7.5) | DSPC (2.5) | Chol (41.5) | PEG-DMG (0.5) |
| 18 | KC3-C17(8:1) (48) | DSPS (7.5) | DPPC (2.5) | Chol (41.5) | PEG-DMG (0.5) |
| 19 | KC3-0A (47.5) | DSPS (7.5) | DSPC (4.5) | Chol (39) | PEG-DMG (1.5) |
| 20 | KC3-0A (47.5) | DPPS (7.5) | DSPC (4.5) | Chol (39) | PEG-DMG (1.5) |
| 21 | KC3-0A (47.5) | DSPS (7.5) | DSPC (2.5) | β-sitosterol (42) | PEG-DMG (0.5) |
| 22 | KC3-0A (50) | DSPS (7.5) | DSPC (2.5) | β-sitosterol (39) | PEG-DMG (1) |
| 23 | KC3-0A (52.5) | DSPS (7.5) | DSPC (2.5) | β-sitosterol (39) | PEG-DMG (1) |
| 24 | KC3-0A (55) | DSPS (7.5) | DSPC (2.5) | β-sitosterol (39) | PEG-DMG (1) |
| 25 | KC3-0A (55) | DSPS (7.5) | DSPC (2.5) | Chol (34) | PEG-DMG (1) |
| 26 | KC3-0A (57) | DSPS (6.5) | DSPC (3.5) | Chol (28) | PEG-DMG (1) |
| | | | DOPE (4) | | |
| 27 | KC3-0A (52.5) | DSPS (7.5) | DSPC (6.5) | Chol (28) | PEG-DMG (1.5) |
| | | | DOPE (4) | | |
| 28 | KC3-0A (48) | DSPS (8) | DSPC (2) | Chol (41.5) | PEG-DMG (0.5) |
| 29 | KC3-PA (48) | DSPS (7) | DSPC (3) | Chol (41) | PEG-DMG (1) |
| 30 | AKG-KC3-01 (45) | DSPS (6) | DSPC (4) | Chol (44) | PEG-DMG (1) |
| 31 | KC3-PA (42.5) | DSPS (7.5) | DSPC (6.5) | Chol (42) | PEG-DMG (1.5) |
| 32 | KC3-0A (48) | DPPS (8) | DSPC (2) | Chol (41.5) | PEG-DMG (0.5) |
| 33 | KC3-C17(8:1) (48) | DSPS (5) | DSPC (5) | Chol (41.5) | PEG-DMG (0.5) |
| 34 | KC3-C17(8:1) (48) | DSPS (7.5) | DSPC (2.5) | Chol (41) | PEG-DMG (1) |

TABLE 15
Exemplary phosphatidyl-L-serine containing LNP formulations (continued)

| | Ionizable | | Additional | | Conjugated |
|---|---|---|---|---|---|
| Formulation | Cationic Lipid | PS (mol | Phospholipid(s) | Sterol | lipid |
| (#) | (mol %) | %) | (mol %) | (mol %) | (mol %) |
| 35 | KC3-C17(8:1) (50) | DSPS (5) | DSPC (5) | Chol (39) | PEG-DMG (1) |
| 36 | KC3-C17(8:1) (50) | DSPS (7.5) | DSPC (2.5) | Chol (39) | PEG-DMG (1) |
| 37 | KC3-OA (48) | DSPS (5) | DSPC (5) | Chol (41.5) | PEG-DMG (0.5) |
| 38 | ALC-0315 (46) | DSPS (5) | DSPC (5) | Chol (43) | PEG-DMG (1) |
| 39 | ALC-0315 (48) | DSPS (5) | DSPC (5) | Chol (41) | PEG-DMG (1) |
| 40 | SM-102 (50) | DSPS (5) | DSPC (5) | Chol (39.5) | PEG-DMG (0.5) |
| 41 | SM-102 (50) | DSPS (7.5) | DSPC (2.5) | Chol (39) | PEG-DMG (1) |
| 42 | SM-102 (48) | DSPS (5) | DSPC (5) | Chol (41) | PEG-DMG (1) |
| 43 | KC3-C17(8:1) (48) | DSPS (7.5) | — | Chol (43.5) | PEG-DMG (1) |
| 44 | KC3-PA (48) | DSPS (7.5) | — | Chol (43.5) | PEG-DMG (1) |
| 45 | AKG-KC3-01 (48) | DSPS (5) | DSPC (5) | Chol (26) | PEG-DMG (1) |
| | | | DOPE (15) | | |
| 46 | AKG-KC3-01 (48) | DSPS (5) | DSPC (5) | Chol (31) | PEG-DMG (1) |
| | | | DOPE (10) | | |
| 47 | AKG-KC3-01 (48) | DSPS (6) | DSPC (4) | Chol (40) | PEG-DMG (2) |
| 48 | AKG-KC3-01 (48) | DSPS (6) | DSPC (4) | Chol (39.5) | PEG-DMG (2.5) |
| 49 | KC3-PA (46) | DSPS (7) | DSPE (5) | Chol (41.5) | PEG-DMG (0.5) |
| 50 | KC3-PA (48) | DSPS (7.5) | eggSM (2.5) | Chol (41.5) | PEG-DMG (0.5) |
| 51 | AKG-KC3-01 (48) | DSPS (5) | HSPC (5) | Chol (41.5) | PEG-DMG (0.5) |
| 52 | KC3-PA (47.5) | DSPS (7.5) | DSPC (2.5) | Chol (42) | PEG-DPG (0.5) |
| 53 | KC3-PA (47) | DSPS (7.5) | DSPC (2.5) | Chol (42) | PEG-DPG (1) |

### Example 16. Determination of Transfection Efficiency in Human Dendritic Cells of LNPs Using mCherry mRNA

Four days prior to transfection, monocytes were isolated using CD14 isolation kit (StemCell) from PBMCs of healthy donors. Purity CD14=90.3%, viability=93.2%. The monocytes were cultured with IL-4 (R&D 1000 IU/mL) and GM-CSF (R&D, 800 IU/mL) in a 6 well dish at 1×106 cells/mL at 37° C., 5% CO2. After 4 days the immature DCs were harvested and seeded in a 96 well round bottom plate at 50,000 cells/well. LNPs were thawed by placing the vials in a 37° C. water bath for 30 seconds, or until the sample had almost fully melted. The vials were immediately placed on ice until use. The LNPs were added to final concentrations of 1 μg/ml and 0.1 μg/mL mRNA. For 1 μg/mL treatment, LNPs were added directly to each well then wells were mixed by pipetting. For 0.1 μg/mL treatment, LNPs were diluted 1:10 in complete media then added to each well and mixed by pipetting. After 4 h a maturation cytokine cocktail was added directly to each well consisting of TNF-α (R&D, 10 ng/ml), IL-1b (R&D, 2 ng/mL), IL-6 (R&D, 1000 IU/mL), and PGE1 (R&D, 1 μg/mL). After 24 h, the cells were centrifuged, washed in PBS and analyzed by flow cytometry for mCherry fluorescence. Gating analysis was performed using CytExpert software.

### Example 17. Stability of Ionizable Cationic Lipids Under Accelerated Oxidation Conditions

The aim of this studies was to compare stability of ionizable cationic lipids under accelerated oxidation.

Ethanol (Sigma-Aldrich, cat #459836) stocks of 1 mM cationic lipids (KC3, KC3-OA, KC3-PA, KC3-C17 and KC3-C17 (C8:1)) were prepared and stored at −20° C. Prior to the experiment, 75 μl of a lipid stock was mixed with 75 μl of ultra-pure water (Rx Biosciences, cat #P01-UPW02-1000) to make 0.5 mM of cationic lipids and 5 mM of linoleic acid respectively. A combined stock in water of 10% H₂O₂ (Sigma-Aldrich, cat #H1009) and 1 mM of Fe(III)Cl (Sigma-Aldrich, cat #372870) was freshly by prepared prior to the experiment. To make 1% final concentration of H₂O₂ and 100 μM Fe(III)Cl, 15 μl of 10% H₂O₂/1 mM Fe(III)Cl stock was added to 135 μl of individual lipids The liposomes and individual lipids were incubated with H₂O₂/Fe(III)Cl at 37° C. and then 5 μl from each sample was taken at different time points (0, 24, 48, 72 and 96 hours) and dissolved in 90 μl of MeOH for HPLC analysis. Degradation of the main lipid peak was analyzed using Thermo Scientific Vanquish Flex UHPLC occupied with Charged Aerosol Detector (CAD) and Phenomenex™ Kinetex C8 column (L=150 mm, D=2.1 mm, Particle Size=1.7 μm, 100 A). The UHPLC operating conditions are listed in Table 24.

TABLE 16
Chromatographic Conditions

| HPLC Instrument | Thermo Scientific Vanquish Flex UHPLC | | |
|---|---|---|---|
| HPLC Column | PhenomenexTM Kinetex C8 column (L = | | |
| | 150 mm, D = 2.1 mm, Particle | | |
| | Size = 1.7 μm, 100 A) | | |
| Column | 50 °C | | |
| Temperature | | | |
| Flow Rate | 0.5 mL/min | | |
| Injection Volume | 5 μL | | |
| CAD | 10 Hz | | |
| Run Time | 11 min | | |
| Sample | 21 °C | | |
| Temperature | | | |
| Sample Solvent | MeOH | | |
| Mobile Phase | Mobile Phase A: 100 mM ammonium acetate | | |
| | in water (pH 4) | | |
| | Mobile Phase B: Methanol | | |
| Mobile phase program: | | | |
| | | Mobile | Mobile |
| | Time, min | Phase A, % | Phase B, % |
| | −0.5 | 18 | 82 |
| | 0 | 18 | 82 |
| | 1 | 16 | 84 |
| | 4 | 10 | 90 |
| | 6 | 0 | 100 |
| | 9 | 0 | 100 |
| | 10 | 18 | 82 |
| | 11 | 18 | 82 |

The data are presented as a percentage of the main lipid peak measured at different time points relative to the lipid peak measured at time zero.

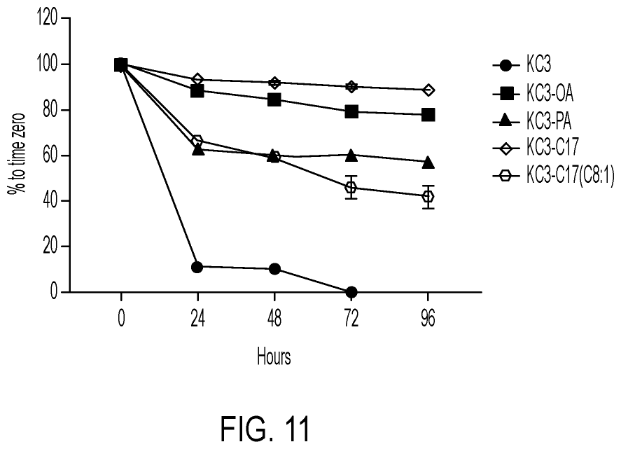

As shown in FIG. 11 and Table 17, KC3 demonstrated the fastest degradation relative to other cationic lipids under the forced oxidation with H₂O₂.

TABLE 17
Effect of hydrogen peroxide on the stability
of individual ionizable cationic lipids

% of main lipid peak to Tₒ

| Lipid | 24 h. | 48 h. | 72 h. |
|---|---|---|---|
| KC3 | 11.2 ± 1.0 | 10.5 ± 0.5 | 0 ± 0 |
| KC3-OA | 88.9 ± 0.5 | 84.6 ± 0.9 | 79.0 ± 0.1 |
| KC3-PA | 62.7 ± 0.1 | 59.2 ± 0.8 | 60.2 ± 0.6 |
| KC3-17 | 92.9 ± 0.3 | 91.8 ± 1.2 | 90.2 ± 1.2 |
| KC3-17 (C8:1) | 66.1 ± 0.6 | 58.4 ± 1.5 | 45.9 ± 4.7 |

### Example 18. Comparison of KC2 and KC3-Family Lipids in PS-Targeted LNP Expression Murine Dendritic Cells

The aim of this study was to explore the effect of different KC2 and KC3 ICLs on transfection efficiency in murine dendritic cells. The KC2 (DLin-KC2-DMA) and KC3 (DLin-KC3-DMA) lipids both have linoleic chains with two olefins each but differ in the ionizable amine group coming off the dioxolane ring, with KC2 having a dimethylaminoethyl group at this position and KC3 has a dimethylaminopropyl group at this position. Two other variants evaluated have monounsaturated alkyl chains with KC3-OA being 18 carbons in length and KC3C17(8:1) having 17 carbons. LNPs were prepared as described in Example 2, characterized for particle size and zeta potential as described in Example 3, and evaluated for transfection efficiency in murine dendritic cells as described in Example 4. The LNPs used the ionizable lipids in Tables 29-30 in the ICL with a constant N/P ratio of 5.25, the DPPS (NH4+ salt) lipid was either 0 or 5 mol % and the DSPC phospholipid constant at 10 or 5 mol % (total of DPPS and DSPC was 10 mol %), and the cholesterol constant at 38.5 mol %.

TABLE 18
Physicochemical properties of LNPs used in Example 18.

| | | Particle Size | |
|---|---|---|---|
| | Particle | (nm) Post- | Encapsulation |
| LNP formulation | Size (nm) | Freeze/Thaw | Efficiency (%) |
| KC2/DSPC/DSPS | 87.8 | | 85.6 ± 4.6 |
| KC3/DSPC | 91.2 | 90.1 | 95.1 ± 5.6 |
| KC3/DSPC/DPPS | 90.5 | 90.2 | 95.0 ± 9.8 |
| KC3-OA/DSPC | 88.4 | 84.7 | 96.0 ± 13.1 |
| KC3-OA/DSPC/DPPS | 97.7 | 97.7 | 96.5 ± 7.3 |
| KC3C17(8:1)/DSPC | 85.3 | 83.0 | 94.4 ± 6.2 |
| KC3C17(8:1)/DSPC/DPPS | 100.2 | 96.8 | 94.7 ± 8.4 |
| KC3C17/DSPC | 73.9 | 77.5 | 95.6 ± 5.5 |
| KC3C17/DSPC/DPPS | 85.2 | 81.4 | 97.0 ± 8.4 |

TABLE 19
mCherry expression in murine dendritic cells

| | [mRNA] | | | vs KC3 |
|---|---|---|---|---|
| LNP formulation | (μg/mL) | MFI | T/NT | LNP (%) |
| Untreated (UT) | | 234 ± 6 | | |
| KC2/DSPC/DSPS | 1 | 13,570 ± 2,324 | | 17.3% |
| KC3/DSPC | 1 | 14,962 ± 711 | | |
| | 0.3 | 6,656 ± 125 | | |
| KC3/DSPC/DPPS | 1 | 78,539 ± 15,239 | 5.32 | |
| | 0.3 | 30,525 ± 2,946 | 4.72 | |
| KC3-OA/DSPC | 1 | 27,513 ± 10,579 | | 185% |
| | 0.3 | 12,719 ± 1,316 | | 194% |
| KC3-OA/DSPC/DPPS | 1 | 163,844 ± 24,614 | 6.00 | 209% |
| | 0.3 | 85,584 ± 10,863 | 6.84 | 282% |
| KC3C17(8:1)/DSPC | 1 | 16,075 ± 1,956 | | 108% |
| | 0.3 | 6,701 ± 440 | | 101% |
| KC3C17(8:1)/DSPC/ | 1 | 145,159 ± 35,387 | 9.15 | 185% |
| DPPS | 0.3 | 61,788 ± 7,404 | 9.52 | 203% |
| KC3C17DSPC | 1 | 344 ± 15 | | 0.8% |
| | 0.3 | 270 ± 8 | | 0.6% |
| KC3C17/DSPC/DPPS | 1 | 428 ± 30 | 1.77 | 0.3% |
| | 0.3 | 330 ± 5 | 2.64 | 0.3% |

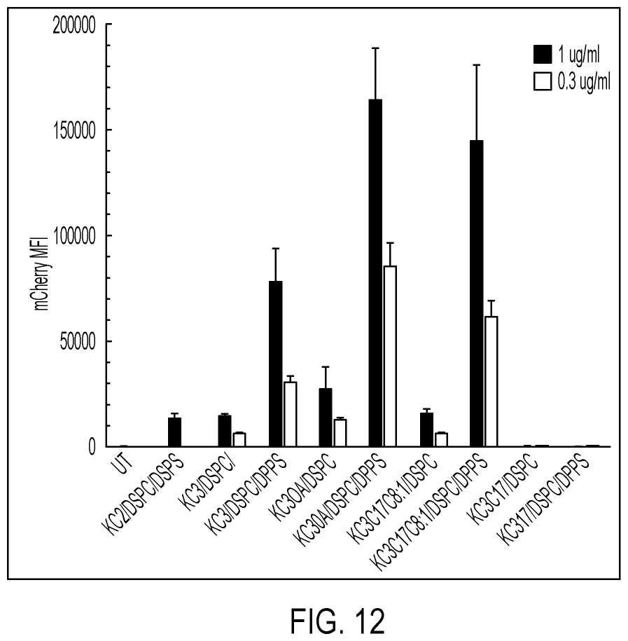

The effect of targeting and ICL choice on transfection activity was evaluated using both nontargeted and 5 mol % DSPS-targeted LNPs (FIG. 12). The five ICLs used in this study had either a diacyl structure that varied in the specific ionizable amine used, and thus it's apparent pKa. This data shows that the two KC3 lipids with a single olefin, KC3OA and KC3C17(8:1) showed the highest activity when incorporated into LNPs. These two lipids when incorporated into 5 mol % DPPS-targeted LNPs showed between 185-282% of the activity of the KC3 lipid containing LNP formulation and 1,087-1,227% of the transfection activity of LNPs containing the KC2 lipid. Previously, Semple and colleagues (Semple et al., (2010) Nature Biotech. 28 (2) 172-176) had shown that KC2 was 6-fold more active compared to KC3, and all the lipids were designed with the understanding that linoleyl lipids with two cis double bonds would be significantly less active based on prior work with DLinDMA (Heyes et al., (2005) J Control Release 107, 276-287), and thus monounsaturated lipids were never evaluated. Here, the data clearly show using two different monounsaturated ICLs, KC3-OA and KC3C17(8:1), that the monounsaturated lipids were clearly superior to lipids containing dilioleyl chains and compared to those containing fully saturated alkyl chains (KC3C17), which was completely inactive, even in the presence of DPPS targeting lipid. The data also show these new lipids were significantly more active than the lead, and most active lipid, KC2 (DLin-KC2-DMA) arising from this early work. Finally, LNPs comprised of both DPPS and one of these two monounsaturated ICLs, showed a 6-10 fold targeting effect compared to similar LNPs without DPPS.

### Example 19. Comparison of KC2 and KC3-Family Lipids in PS-Targeted LNP Expression Human Dendritic Cells

The aim of this study was to explore the effect of different KC2 and KC3 ICLs on transfection efficiency in human dendritic cells. The KC2 (DLin-KC2-DMA) and KC3 (DLin-KC3-DMA) lipids both have linoleic chains with two olefins each but differ in the ionizable amine group coming off the dioxolane ring, with KC2 having a dimethylaminoethyl group at this position and KC3 has a dimethylaminopropyl group at this position. Three other variants evaluated have monounsaturated alkyl chains with KC3-OA being 18 carbons in length, KC3-PA having 16 carbons, and KC3C17(8:1) having 17 carbons. The aim of this study was also to evaluate the impact of the PC component on transfection by comparing the fully saturated C16 DPPC and the fully saturated DSPC using both KC3-OA and KC3C17(8:1) containing LNPS. LNPs were prepared as described in Example 2, characterized for particle size and zeta potential as described in Example 3, and evaluated for transfection efficiency in human dendritic cells as described in Example 16. The LNPs used the ionizable lipids in Tables 31-33 with the ICL held constant at an N/P ratio of 5.25 and 48 mol %, the DPPS (NH4+ salt) lipid was included at 5 mol % and the DSPC phospholipid constant at 5 mol % (total of DPPS and DSPC/DPPC was 10 mol %), and the cholesterol constant at 38.5 mol %. The final ALC-0315/DSPC formulation was composed of 46.3 mol % ALC-0315, 9.4 mol % DSPC, 42.7 mol % cholesterol and 1.56 mol % PEG-DMG with a N/P of 6.2. The final SM-102/DSPC formulation was composed of 50 mol % SM-102, 10 mol % DSPC, 38.5 mol % cholesterol and 1.5 mol % PEG-DMG with a N/P of 5.

TABLE 19
Physicochemical properties of LNPs used in Example 19.

| | | Particle | |
|---|---|---|---|
| | | Size (nm) | |
| | Particle | Post-Freeze/ | Encapsulation |
| LNP formulation | Size (nm) | Thaw | Efficiency (%) |
| KC2/DSPC/DSPS | 71.8 | 69.6 | 85.4 ± 1.5 |
| KC3-01/DSPC/DPPS | 67.9 | 71.7 | 84.3 ± 3.6 |
| KC3/DSPC/DPPS | 103.4 | 100.3 | 91.5 ± 6.2 |
| KC3-OA/DSPC/DPPS | 87.9 | 87.7 | 92.2 ± 8.6 |
| KC3-OA/DPPC/DPPS | 89.8 | 98.2 | 91.7 ± 6.3 |
| KC3-PA/DSPC/DPPS | 104.7 | 98.0 | 90.1 ± 10.3 |
| KC3C17(8:1)/DSPC/DPPS | 91.6 | 92.7 | 91.7 ± 5.3 |
| KC3C17(8:1)/DPPC/DPPS | 86.6 | 88.7 | 92.9 ± 2.6 |
| ALC-0315/DSPC | 69.8 | 71.5 | 88.0 ± 4.1 |
| SM-102/DSPC | 82.1 | 83.4 | 92.4 |

TABLE 20
mCherry expression in human dendritic
cells following incubation at 1 μg/ml

| | | vs KC3 | vs DPPC |
|---|---|---|---|
| | | LNP | LNP |
| LNP formulation | MFI | (fold) | (fold) |
| Untreated (UT) | 2,279 ± 129 | | |
| KC2/DSPC/DSPS | 11,150 ± 1,556 | 0.08 | |
| KC3-01/DSPC/DPPS | 1,149,875 ± 111,037 | 10.08 | 2.35 |
| KC3-OA/DSPC/DPPS | 963,929 ± 95,910 | 8.44 | |
| KC3-OA/DPPC/DPPS | 412,204 ± 37,299 | 3.60 | |
| KC3-PA/DSPC/DPPS | 757,849 ± 95,345 | 6.64 | |
| KC3C17(8:1)/DSPC/ | 534,708 ± 80,921 | 4.68 | 3.60 |
| DPPS | | | |
| KC3C17(8:1)/DPPC/ | 149,980 ± 34,195 | 1.30 | |
| DPPS | | | |
| KC3/DSPC/DPPS | 116,155 ± 30,093 | 1.00 | |
| ALC-0315/DSPC | 10,327 ± 1,916 | 0.07 | |
| SM-102/DSPC | 258,967 ± 16,828 | 2.25 | |

TABLE 21
mCherry expression in human dendritic
cells following incubation at 0.1 μg/ml

| | | vs KC3 | vs DPPC |
|---|---|---|---|
| | | LNP | LNP |
| LNP formulation | MFI | (fold) | (fold) |
| Untreated (UT) | 2,502 ± 293 | | |
| KC2/DSPC/DSPS | 3,048 ± 193 | 0.09 | |
| KC3-01/DSPC/DPPS | 46,489 ± 4,173 | 7.45 | |
| KC3-OA/DSPC/DPPS | 44,197 ± 4,407 | 7.06 | 2.61 |
| KC3-OA/DPPC/DPPS | 18,485 ± 1,488 | 2.71 | |
| KC3-PA/DSPC/DPPS | 35,177 ± 3,396 | 5.53 | |
| KC3C17(8:1)/DSPC/DPPS | 25,044 ± 2,144 | 3.82 | 2.82 |
| KC3C17(8:1)/DPPC/DPPS | 10,499 ± 979 | 1.35 | |
| KC3/DSPC/DPPS | 8,409 ± 660 | 1.00 | |
| ALC-0315/DSPC | 3,783 ± 1,090 | 0.22 | |
| SM-102/DSPC | 9,177 ± 1,494 | 1.13 | |

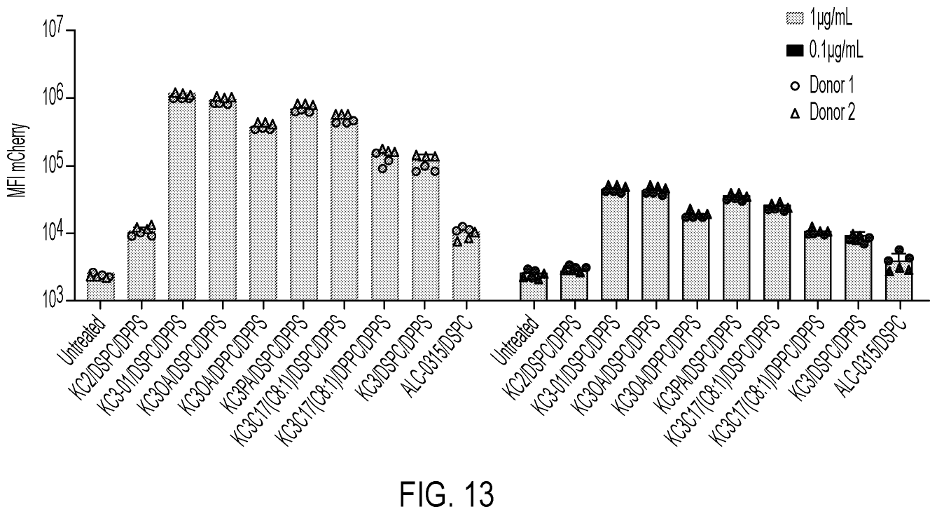

The effect of targeting and ICL choice on transfection activity was evaluated using both nontargeted and 5 mol % DPPS-targeted LNPs (FIG. 13). The five ICLs used in this study had either a diacyl structure that varied in the specific ionizable amine used. This data shows that the three KC3 lipids with a single olefin, KC3OA, KC3-PA and KC3C17(8:1), or the polyunsaturated KC3-01 lipid with four methylenes between the two olefins showed the highest activity when incorporated into LNPs (Tables 20 and 21). The four lipids showed a 4.7-10.8 fold increase in transfection activity compared to KC3-containing LNPs in human dendritic cells at 1 μg/ml and 3.8-7.5 fold increase at 0.1 μg/mL. The increase was a further 10-12 fold higher when compared to targeted LNPs containing KC2. Both KC2 and KC3 containing linoleoyl alkyl chains (two olefins separated by one methylene). Previously, Semple (Semple et al., (2010) Nature Biotech. 28 (2) 172-176) had shown that KC2 was 6-fold more active compared to KC3, and all the lipids were designed with the understanding that linoleyl lipids with two cis double bonds would be significantly less active based on prior work with DLinDMA (Heyes et al., (2005) J Control Release 107, 276-287), and thus monounsaturated lipids were never evaluated. Here, the data clearly show using three different monounsaturated ICLs, KC3-OA, KC3-PA, and KC3C17(8:1), that the monounsaturated lipids were clearly superior to lipids containing dilioleyl chains. However, the largest improvement at both concentrations came when using the KC3-01 ICL with four methylenes between two olefins. The data also show these new lipids were significantly more active than the lead, and most active lipid, KC2 (DLin-KC2-DMA) arising from this early work and compared to LNPs containing either ALC-0315 or SM-102. Finally, two of the DPPS-targeted LNPs comprised of both DPPS and either KC3-OA or KC3-C17(8:1) ICLs, were evaluated with either the C16 DPPC or the C18 DSPC as the phosphatidylcholine component. For both sets of formulations, the DSPC-containing formulation showed higher transfection activity in human dendritic cells compared to the DPPC formulation, 2.61-2.82-fold higher 0.1 μg/mL and 2.35-3.60-fold higher at 1 μg/mL. This suggests that the DSPC containing LNPs provide for greater transfection activity when compared to lower phase transition lipids like DPPC.

### Example 20. Impact of ICL Concentration in PS-Targeted LNPs on Expression in Human Dendritic Cells

The aim of this study was to explore the effect of KC3-OA concentration in DSPS-targeted LNPs on transfection efficiency in human dendritic cells. The concentrations of KC3-OA were varied from 46-54 mol %, while keeping the DSPS and DSPC concentrations constant at 5 mol % each and PEG-DMG at 1.5 mol %. The increase in % KC3OA was commensurate with a proportional reduction in the % of cholesterol. ALC-0315 and SM-102 comparator formulations were also evaluated. The final ALC-0315/DSPC formulation was composed of 46.3 mol % ALC-0315, 9.4 mol % DSPC, 42.7 mol % cholesterol and 1.56 mol % PEG-DMG with a N/P of 6.2. The final SM-102/DSPC formulation was composed of 50 mol % SM-102, 10 mol % DSPC, 38.5 mol % cholesterol and 1.5 mol % PEG-DMG with a N/P of 5.

LNPs were prepared as described in Example 2, characterized for particle size and zeta potential as described in Example 3, and evaluated for transfection efficiency in human dendritic cells as described in Example 16.

TABLE 22
Physicochemical properties and characterization of DSPS-targeted
KC3-OA LNPs containing various concentrations of KC3-OA

| | | Particle Size | |
|---|---|---|---|
| | Particle | (nm) Post- | Encapsulation |
| LNP formulation | size (nm) | Freeze/Thaw | Efficiency (%) |
| SM-102/DSPC | 71.0 | 74.1 | 84.1 ± 10.7 |
| ALC-0315/DSPC | 63.0 | 62.3 | 89.0 ± 5.9 |
| KC3-OA (46)/DSPC/DSPS | 101.9 | 110.0 | 94.1 ± 10.0 |
| KC3-OA(48)/DSPC/DSPS | 96.9 | 110.6 | 93.8 ± 16.8 |
| KC3-OA(50/DSPC/DSPS | 109.0 | 108.0 | 93.1 ± 14.4 |
| KC3-OA(52)/DSPC/DSPS | 108.6 | 110.9 | 93.8 ± 10.0 |
| KC3-OA(54)/DSPC/DSPS | 121.2 | 122.8 | 93.8 ± 17.0 |

All DSPS-targeted KC3-OA formulations showed a particle size around 100-120 nm and greater than 90% encapsulation efficiency. All particles were stable to freezing and thawing at −80° C. There was no apparent impact of the concentration of KC3-OA used in the range of 46-54 mol % on these properties.

TABLE 23
mCherry expression in human dendritic cells following
incubation at 1 μg/ml for DSPS-targeted KC3-OA
LNPs containing various concentrations of KC3-OA

| | | vs SM- | vs ALC- |
|---|---|---|---|
| | | 102 | 0315 |
| LNP formulation | MFI | (fold) | (fold) |
| Untreated (UT) | 1,343 ± 104 | | |
| SM-102/DSPC | 4,242 ± 1,942 | | |
| ALC-0315/DSPC | 3,760 ± 1,625 | | |
| KC3-OA (46)/DSPC/DSPS | 100,521 ± 44,930 | 34 | 41 |
| KC3-OA(48)/DSPC/DSPS | 116,994 ± 52,141 | 40 | 48 |
| KC3-OA(50/DSPC/DSPS | 107,911 ± 46,614 | 37 | 44 |
| KC3-OA(52)/DSPC/DSPS | 67,845 ± 37,779 | 23 | 28 |
| KC3-OA(54/DSPC/DSPS | 101,338 ± 46,945 | 34 | 41 |

TABLE 24
mCherry expression in human dendritic cells following
incubation at 0.1 μg/ml for DSPS-targeted KC3-OA
LNPs containing various concentrations of KC3-OA

| | | vs SM- | vs ALC- |
|---|---|---|---|
| | | 102 | 0315 |
| LNP formulation | MFI | (fold) | (fold) |
| Untreated (UT) | 1,295 ± 116 | | |
| SM-102/DSPC | 1,492 ± 26 | | |
| ALC-0315/DSPC | 1,405 ± 28 | | |
| KC3-OA (46)/DSPC/DSPS | 6,915 ± 1,659 | 29 | 51 |
| KC3-OA(48)/DSPC/DSPS | 10,491 ± 3,164 | 47 | 84 |
| KC3-OA(50/DSPC/DSPS | 11,033 ± 3,043 | 49 | 89 |
| KC3-OA(52)/DSPC/DSPS | 8,393 ± 2,447 | 36 | 65 |
| KC3-OA(54)/DSPC/DSPS | 7,496 ± 1,855 | 31 | 57 |

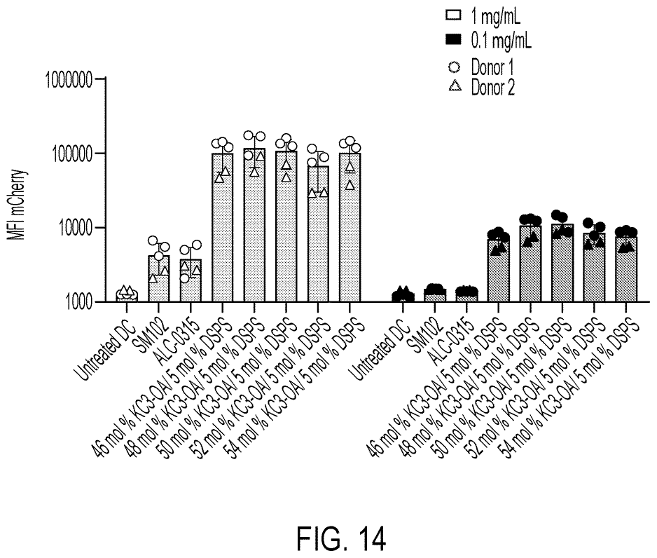

The effect of targeting and ICL choice on transfection activity was evaluated using 5 mol % DSPS-targeted LNPs comprised of KC3-OA at concentrations ranging from 46-54 mol % (FIG. 14). This targeted composition was highly active at all concentrations of KC3-OA at both 0.1 and 1 μg/mL, and only showed a slight peak at 48-50 mol % KC3-OA. However, all variations of this DSPS-targeted KC3-OA LNP formulation were significantly more active than either SM-102/DSPC or ALC-0315/DSPC controls (Tables 34-36).

### Example 21. Impact of N/P Ratio in PS-Targeted LNPs on Expression in Human Dendritic Cells

The aim of this study was to explore the effect of varying the N/P ratio in DSPS-targeted KC2-01 LNPs on transfection efficiency in human dendritic cells. The N/P ratio was varied from 4-7, while keeping the DSPS and DSPC concentrations constant at 5 mol % each, the concentration of KC2-01 constant at 50 mol %, and PEG-DMG at 1.5 mol %. ALC-0315 and SM-102 comparator formulations were also evaluated. The final ALC-0315/DSPC formulation was composed of 46.3 mol % ALC-0315, 9.4 mol % DSPC, 42.7 mol % cholesterol and 1.56 mol % PEG-DMG with a N/P of 6.2. The final SM-102/DSPC formulation was composed of 50 mol % SM-102, 10 mol % DSPC, 38.5 mol % cholesterol and 1.5 mol % PEG-DMG with a N/P of 5.

LNPs were prepared as described in Example 2, characterized for particle size and zeta potential as described in Example 3, and evaluated for transfection efficiency in human dendritic cells as described in Example 16.

TABLE 25
Physicochemical properties and characterization of DSPS-targeted
KC2-O1 LNPs containing KC2-O1 at various N/P ratios

| | | Particle Size | |
|---|---|---|---|
| | | (nm) Post- | Encapsulation |
| LNP formulation | Particle size | Freeze/Thaw | Efficiency (%) |
| KC2-O1 (N/P = 4) | 85.4 | 91.3 | 83.2 ± 4.7 |
| KC2-O1/DSPS (N/P = 4) | 84.1 | 90.2 | 85.5 ± 3.9 |
| KC2-O1 (N/P = 5) | 84.9 | 87.0 | 88.0 ± 4.7 |
| KC2-O1/DSPS (N/P = 5) | 80.91 | 88.0 | 88.3 ± 4.0 |
| KC2-O1 (N/P = 6) | 81.1 | 84.0 | 88.0 ± 1.3 |
| KC2-O1/DSPS (N/P = 6) | 82.2 | 88.0 | 89.6 ± 2.7 |
| KC2-O1 (N/P = 7) | 75.7 | 84.0 | 89.0 ± 3.1 |
| KC2-O1/DSPS (N/P = 7) | 79.5 | 81.5 | 88.8 ± 4.1 |

All DSPS-targeted KC2-O1 formulations showed a particle size around 76-85 nm and greater than 80% encapsulation efficiency. All particles were stable to freezing and thawing at −80° C. There was no apparent impact of the N/P used in the range of 4-7 on these properties.

TABLE 26
mCherry expression in human dendritic cells following
incubation at 0.1 μg/ml for DSPS-targeted KC2-O1
LNPs containing KC2-O1 at various N/P ratios

| LNP formulation | MFI | T/NT | vs N/P = 4 |
|---|---|---|---|
| Untreated (UT) | 1,245 ± 3 | | |
| KC2-O1 (N/P = 4) | 2,077 ± 40 | | |
| KC2-O1/DSPS (N/P = 4) | 12,466 ± 2,359 | 13.5 | 1 |
| KC2-O1 (N/P = 5) | 1,966 ± 163 | | |
| KC2-O1/DSPS (N/P = 5) | 35,574 ± 13,780 | 47.6 | 3.1 |
| KC2-O1 (N/P = 6) | 3,389 ± 1,157 | | |
| KC2-O1/DSPS (N/P = 6) | 29,617 ± 3,345 | 13.2 | 2.5 |
| KC2-O1 (N/P = 7) | 5,852 ± 1,961 | | |
| KC2-O1/DSPS (N/P = 7) | 39,097 ± 6,398 | 8.2 | 3.4 |

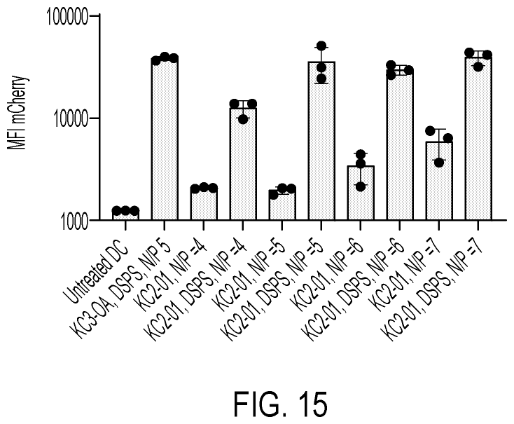

The effect of targeting and N/P ratio on transfection activity was evaluated using 5 mol % DSPS-targeted LNPs comprised of KC2-O1 at ratios of 4-7 (FIG. 15). This targeted composition was highly active at all N/P ratios, but did display an approximately 3-fold drop off going from N/P of 5 to N/P of 4. The targeted to nontargeted (T/NT) ratio displayed a clear peak at N/P=5 (Table 26).

### Example 22. Impact of Ionizable Lipid on Immunogenicity of SARS-COV-2 Spike Protein mRNA Vaccine Constructs

Mice and study design. Female BALB/c mice were purchased from Charles River Laboratories, allowed to acclimate in the vivarium for at least 4 days, and were 6-8 weeks old at the start of the study. On study day (SD) 0 mice were injected intramuscularly in the left rear thigh with 1 μg of vaccine candidate (quantity refers to mRNA) in a volume of 50 μL. Study groups consisted of 5 mice and included vehicle control, comparator vaccines, and experimental vaccine candidates. Mice were given a second injection of the same vaccine candidate 21 days later. Blood was collected via submandibular puncture or terminally via cardiac puncture and serum or plasma was isolated from mice on SD 21 and 34 (+13 days post boost). Samples were stored at −80° C. until analysis for antibody titers.

Design and preparation of mRNA. mRNA encoding the SARS-COV-2 full length spike protein and flanked with the same UTRs used in the BNT162b2 (Comirnaty) vaccine was purchased from Vernal Biosciences. All uridine nucleosides were substituted with N1-methyl-pseudouridine. To produce the mRNA, a synthetic gene encoding the mRNA sequence (VRN029; SEQ ID NO: 211) was cloned into a DNA plasmid. The synthetic gene was comprised of an RNA promoter, a 5′ untranslated region, the SARS-COV2 Spike protein receptor binding domain, a 3′ untranslated region, and a poly(A) tail region of approximately 120 As. The plasmid was propagated and expanded in a culture of E. coli and then isolated from the clarified E. coli lysate via anion exchange chromatography. The purified plasmid was linearized using a type IIs restriction enzyme that cut at a site at the end of the poly(A) sitail encoding region. That plasmid was then incubated in a buffer with nucleotide triphosphates, RNA polymerase, and RNase inhibitor. To stop the reaction, DNase I was added to digest the linear plasmid template. The uncapped RNA was then purified using chromatography and then incubated in another buffer with GTP, S-adenosylmethionine, a guanalyltransferase, 2′-O-methyltransferase, and RNase inhibitor. The capped mRNA was then purified using chromatography, buffer exchanged into water, and filled into vials.

Generation of lipid nanoparticles (LNP) containing mRNA. Stock solutions of each lipid were prepared. Ionizable lipids were weighed out in 4 mL glass vials (Thermo B7999-2) and dissolved in ethanol (Sigma-Aldrich 200 proof, RNase free) to a final concentration of 10 mM. Other lipids such as DSPC (Avanti Polar Lipids), Cholesterol (Dishman) and PEG-DMG (NOF) were weighed out and dissolved in ethanol to a concentration of 1 mM. DSPS-Na (NOF) was dissolved in methanol (Sulpelco, Omnisolve) at a concentration of 1 mM and briefly heated to 70° C. to complete its dissolution. DPPS-NH4 (Avanti Polar Lipids) was dissolved directly in ethanol and incorporated at room temperature along with the other lipid components.

Lipid mixtures for each individual LNP were prepared by adding the desired volume of each lipid stock solution to a new vial, adding ethanol if needed to achieve a final volume of 1.2 mL. All formulations had 5 mol % of the PC and PS component, 40.5 mol % of cholesterol, 1.5 mol % of PEG-DMG, 48 mol % of the ICL, and a constant N/P ratio of 5.25. For example, a LNP formulation of KC3-PA/DPPC/DPPS/Chol/PEG-DMG (48/5/5/40.5/1.5 mol %), with an N/P of 5.25 contained 1575 nmol KC3-PA, 164.1 nmol DPPC, 164.1 nmol DPPS, 1296.1 nmol Chol and 82 nmol PEG-DMG for every 100 μg of mRNA used. mRNA solutions were prepared by thawing frozen mRNA (SARS-COV-2 spike mRNA, Vernal) vials and diluting mRNA in 6.25 mM sodium acetate (pH 5.0) to a final concentration of 0.033 mg/mL, where the concentration is confirmed by absorbance on a Nanodrop.

To prepare LNPs, a NanoAssemblr Benchtop microfluidic device (from Precision Nanosystems) was used. If LNPs contained DSPS, the heating block accessory set to 70° C. was used, otherwise LNPs were mixed at room temperature. 3 mL of mRNA solution was loaded into a 3 mL disposable syringe (BD 309656) and 1 ml of lipid mixture in a 1 ml syringe (BD309659) and placed in the NanoAssemblr heating block for 4 min prior to mixing. LNP formation was achieved by pumping the liquid streams through a disposable microfluidics cassette at 3:1 aqueous:alcohol volume ratio at 6 mL/min mixing speed. After mixing, 3.6 mL of LNP mixture was collected, while the initial mixed volume of 0.35 mL and last 0.05 mL of mix was discarded. Ethanol was removed by buffer exchange using SpectraPor dialysis tubing (12-14k MWCO) in PBS (Cytivia, SH30256.01). LNPs were typically exchanged into PBS, pH 7.4 and then 15 mM Tris, pH 7.4, 20% sucrose, concentrated to 20-50 μg/mL mRNA, sterile filtered (Thermo Nalgene 0.2 um #720-1320) prior to freezing by immersion in liquid nitrogen for 5 min and long-term storage at −20° C. For this study, samples were concentrated to >40 μg/mL mRNA, and diluted with varying volumes of 15 mM Tris, 20% Sucrose, pH 7.4 to a target concentration of 40 μg mRNA and then frozen on LN2. Characterization of LNPs was undertaken after an aliquot of the LNPs were thawed and diluted 1:1 (vol:vol) with 15 mM Tris, pH 7.4 such that the final concentration was 20 μg/mL mRNA in 15 mM Tris, 10% sucrose, pH 7.4. This simulated the conditions of sample preparation that were performed prior to dosing the animals with an injection of 1 μg mRNA in 50 μL volume via IM injection into a hind limb.

LNP Characterization. mRNA encapsulation and mRNA concentration within the LNPs was measured using a Ribogreen assay. Nanoparticle size and zeta potential were measured by a zetasizer (Malvern).

SARS-COV-2 anti-spike antibody titers. A standard indirect ELISA was performed to analyze serum samples for total IgG binding antibodies to the SARS-COV-2 spike protein. Nunc MaxiSorp 96-well plates were coated with 75 μL of SARS-COV-2 (Wuhan-Hu-1) spike protein (Sino Biological, cat. no. 40589-V08B1) diluted to 5 nM in 1×PBS, pH 7.4, covered and incubated statically for 16-18 hrs at 4° C. Unbound coating antigen was removed by washing plates 3× with 300 μL PBS+0.05% Tween-20 using a BioTek 405 TS plate washer. Plates were then blocked in PBS+5% w/v non-fat skim milk (Research Products International, cat. no. M17200-1000.0) for 1 hr at 37° C. Test and positive control samples were diluted in assay diluent (PBS, 0.05% Tween-20, 1% w/v non-fat skim milk) to a starting dilution of 1:20 or 1:40 followed by four-fold serial dilutions using U-bottom dilution plates. Once blocking was completed, blocking buffer was removed by inversion, plates were blotted on paper towels, and each sample was plated in duplicates. Plates were statically incubated for 2 hr at 37° C., followed by washing 3× with 300 μL. 100 μL of secondary detection antibody (goat anti-mouse-HRP IgG, Abcam, cat. no. ab6789) in assay diluent was added to each well at a dilution of 1:10,000 (1.33 nM concentration). Plates were incubated statically for 30 min at room temperature, and unbound antibodies were subsequently removed by plate inversion then washed 3× 300 μL. To develop, 100 μL of room temperature 1-Step Ultra TMB substrate (ThermoFisher, cat. no. 34028) was added to each well and the reaction was stopped after 10 min with 50 μL of TMB stop solution (ThermoFisher cat. no. SS04). The plates were read within 30 min at 450 nm with a BioTek Synergy Neo 2 spectrophotometer. Titers were defined as the reciprocal of the dilution that generated an absorbance signal on the linear part of the titration curve.

TABLE 27
Physicochemical properties of LNPs used in evaluating the immunogenicity
of SARS-CoV-2 spike protein mRNA vaccine construct

| | Particle | Zeta | Zeta | |
|---|---|---|---|---|
| | Size | Potential | Potential | Encapsulation |
| LNP Formulation | (nm) | (mV) pH 5 | (mV) pH 7 | Efficiency (%) |
| KC3/DPPS-NH4/DPPC/ | 98.2 | 5.2 | −3.2 | 87.4 ± 7.4 |
| PEG-DMG | | | | |
| KC3-OA/DPPS-NH4/DPPC/ | 73.7 | 11.8 | −2.6 | 91.2 ± 5.5 |
| PEG-DMG | | | | |
| KC3-OA/DSPS/DSPC/ | 97.3 | 7.4 | −3.7 | 87.7 ± 5.9 |
| PEG-DMG | | | | |
| KC3-PA/DPPS-NH4/DPPC/ | 89.7 | 8.4 | −1.0 | 88.0 ± 4.3 |
| PEG-DMG | | | | |
| KC3-PA/DSPS/DSPC/ | 87.8 | 9.0 | −2.9 | 91.8 ± 8.2 |
| PEG-DMG | | | | |

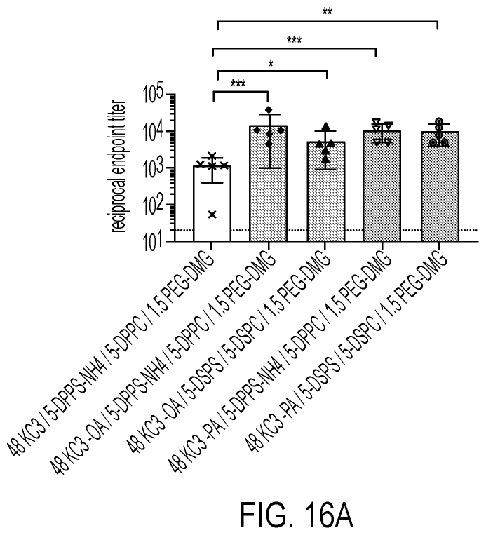

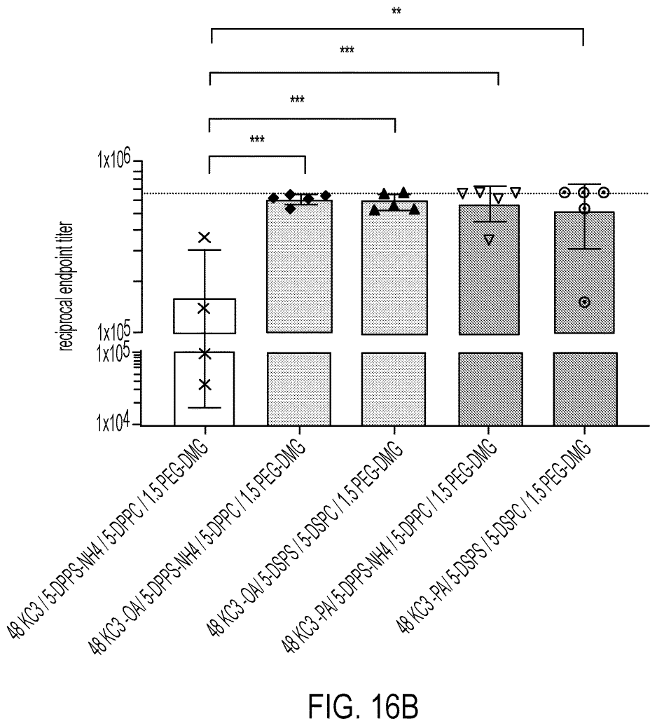

To evaluate the impact of ionizable lipid composition on mRNA-LNP immunogenicity, BALB/c mice were immunized with mRNA-LNPs containing different ionizable lipids (KC3, KC3-OA, and KC3-PA). Blood plasma was collected on day 21 post prime and on day 13 post boost (day 34 of study). Total anti-spike binding IgG antibody titers were determined via ELISA. Reciprocal antibody titer data were log-transformed prior to statistical analysis. Groups were compared using one-way ANOVA with a Dunnett's multiple comparisons post-test. LNPs prepared using the ionizable lipid KC3-OA or KC3-PA induced a superior antibody response than LNPs containing the dilinoleoyl KC3 (DLin-KC3-DMA or just KC3) at both day 21 post prime (FIG. 16A) and day 13 post boost (study day 34) (FIG. 16B).

### Example 23. Preparation of Ammonium Salts of DPPS and DSPS

Dissolve 1.5 g PS sodium salt (DPPS-Na or DSPS-Na) in 500 ml of CHCl₃:Methanol:Water (CMW) 1:3:1 by volume. with 1M NH₄Cl in the water, to make a single-phase solution in a separatory funnel. Swirl 3-5 minutes, apply minimal heat if PS is not completely dissolved. Add CHCl3 and distilled water (approximately 200 mL each) and shake the funnel 2-3 minutes until a clear two-phase partition is achieved. Collect the bottom layer and re-partition with CMW 1:1:1 (by volume) with 1M NH₄Cl in the water. Collect the bottom layer and wash it 2× with fresh methanol-water (the volume of methanol is 50% of the volume of CHCl₃ and the volume of water is 20% of the total volume of chloroform and methanol) until a clear two-phase partition is achieved. Remove solvents under vacuum and freeze-dry from cyclohexane. Yield 1.2 g.

### Example 24. Apparent Solubility of Phosphatidylserine Salts in Ethanol

Apparent solubilities of phosphatidylserine salts in ethanol were determined by shake flask technique. Sodium and ammonium salts of phosphatidylserines in the powder form were obtained from Avanti Polar Lipids (Alabama, USA). Aliquots of phosphatidylserine salt powder were placed in 12×75 borosilicate glass tubes in triplicate. Three mL of 200 proof ethanol (catalog number E-7023, MilliporeSigma, USA) were added, the tubes were closed with polyethylene snap caps, agitated using vortex mixer for 10-15 s to obtain uniform suspensions, and placed in a horizontal position on a rocking platform at room temperature (20-22° C.) overnight. The ambient temperature at the end of incubation was 20.4-20.9° C. After 24 hours incubation, the suspensions were visually checked for the presence of a solid phase, allowed to settle under gravity, and the supernatant solutions were passed through a 0.2-μm PTFE syringe membrane filters. The first 0.5 ml of the filtrate was discarded, the next 1 ml was collected, and phosphatidylserine in the filtrates was quantified by phospholipid phosphate assay as follows: 20 μL aliquots of the filtrate were taken in triplicate into glass tubes, ethanol was evaporated in a stream of argon at 70° C., and the residue was digested in a mixture of 60 μL concentrated sulfuric acid and 20 μL 30% hydrogen peroxide for 10 min at 180-190° C. The digested samples were diluted with 1 mL of deionized water, 20 μL of 10% sodium sulfite was added to destroy any residual peroxide, and the samples were incubated on a boiling water bath for 15 min to hydrolyze any condensed phosphate species. The samples were chilled down to room temperature, mixed with 0.2 mL of 2% (w/w) ammonium molybdate solution and 20 μL of 10% (w/w) ascorbic acid solution, incubated on a boiling water bath for 10 min, and chilled down in a water bath at room temperature. The phosphate concentration was determined from the optical density of the formed blue phosphomolybdic acid at 825 nm using five-point standard curve from concurrently run standards prepared from the NIST-traceable commercial phosphate standard solution diluted to cover the range of 0-2 mM phosphate (coefficient of determination R²>0.9999). Solubility of phosphatidylserine salts was expressed in molarity units of phosphatidylserine (Table 40). The data are average±SD (N=3).

TABLE 28
Solubility of phosphatidylserine salts
in 100% ethanol at room temperature.

| Phosphatidylserine salt | Solubility, mM phosphatidylserine |
|---|---|
| DSPS sodium | 0.111 ± 0.009 |
| DPPS sodium | 0.201 ± 0.004 |
| DSPS ammonium | 0.034 ± 0.004 |
| DPPS ammonium | 0.865 ± 0.060 |

Ammonium salt of dipalmitoylphosphatidylserine (DPPS) unexpectedly showed several-fold higher ethanol solubility than ammonium salt of distearoylphosphatidylserine (DSPS) or sodium salts of any of these compounds.

### Example 25. Impact of PEG-Stearic Acid, N/P, and ICL on Transfection Efficiency in Human Dendritic Cells

The aim of this study was to explore the effect of varying the KC3-OA concentration and N/P ratio in DSPS-targeted LNPs on transfection efficiency in human dendritic cells. In addition, the effect of replacing PEG-DMG with PEG-stearic acid (PEG-SA) (BroadPharm, BP-26262) at 1 and 3 mol % total lipid was compared.

The concentration of KC3-OA was varied from 45-48 mol %, while keeping the DSPS and DSPC concentrations constant at 5 mol % each and PEG-DMG at 1.5 mol. The increase in % KC3OA was commensurate with a proportional reduction in the % of cholesterol. The N/P was varied from 5-6.5 in 0.5 increments while keeping 45 mol % KC3OA constant. In certain cases, PEG-DMG was replaced with PEG-stearic acid. ALC-0315 and SM-102 comparator formulations were also evaluated. The final ALC-0315/DSPC formulation was composed of 46.3 mol % ALC-0315, 9.4 mol % DSPC, 42.7 mol % cholesterol and 1.56 mol % PEG-DMG with a N/P of 6.2. The final SM-102/DSPC formulation was composed of 50 mol % SM-102, 10 mol % DSPC, 38.5 mol % cholesterol and 1.5 mol % PEG-DMG with a N/P of 5.

LNPs were prepared as described in Example 2, characterized for particle size and zeta potential as described in Example 3, and evaluated for transfection efficiency in human dendritic cells as described in Example 16.

TABLE 29
Physicochemical properties and characterization of DSPS-targeted
KC3-OA LNPs at various N/P ratios, various ICL concentrations,
and PEG-SA concentrations used in Example 28

| | | Zeta | Zeta | |
|---|---|---|---|---|
| | Particle | Potential | Potential | Encapsulation |
| LNP formulation | size (nm) | (mV) pH 5 | (mV) pH 7 | Efficiency (%) |
| KC3-OA, 5% DSPS | 85.2 | 13.9 | −1.9 | 90.3 ± 6.4 |
| SM102 | 71.0 | 6.0 | −3.5 | 84.1 ± 10.7 |
| ALC-0315 | 63.0 | 5.2 | −5.2 | 89.0 ± 5.9 |
| 45 mol % KC3-OA, 5% | 90.9 | 15.4 | −2.0 | 93.9 ± 2.4 |
| DSPS (N/P = 5) | | | | |
| 45 mol % KC3-OA, 5% | 91.1 | 14.4 | −0.48 | 92.9 ± 1.7 |
| DSPS (N/P = 5.5) | | | | |
| 45 mol % KC3-OA, 5% | 81.2 | 12.5 | −1.9 | 92.8 ± 6.3 |
| DSPS (N/P = 6) | | | | |
| 45 mol % KC3-OA, 5% | 73.0 | 16.9 | −1.6 | 92.8 ± 7.6 |
| DSPS (N/P = 6.5) | | | | |
| 43% KC3-OA, 5% | 84.3 | 20.8 | −0.2 | 93.0 ± 4.2 |
| DSPS (N/P = 5) | | | | |
| 48% KC3-OA, 5% | 102.6 | 19.6 | −2.1 | 94.4 ± 4.5 |
| DSPS (N/P = 5) | | | | |
| 45% KC3-OA, 5% | 141.7 | 19.6 | −1.6 | 93.8 ± 2.9 |
| DSPS, 1% PEG-SA | | | | |
| (N/P = 5) | | | | |
| 45% KC3-OA, 5% | 123.1 | 17.0 | −3.3 | 91.7 ± 4.8 |
| DSPS, 3% PEG-SA | | | | |
| (N/P = 5) | | | | |
| 45% KC3-OA, 5% | 163 | 23.3 | −1.5 | 92.9 ± 1.7 |
| DSPS, 1% PEG-SA | | | | |
| (N/P = 6) | | | | |
| 45% KC3-OA, 5% | 142.2 | 17.2 | −2.3 | 92.6 ± 3.6 |
| DSPS, 3% PEG-SA | | | | |
| (N/P = 6) | | | | |

All DSPS-targeted KC3-OA formulations showed a particle size about 90 nm and greater than 90% encapsulation efficiency when formulated with 1.5 mol % PEG-DMG. However, the sized increased significantly to about 140 nm when substituting 1.5 mol % PEG-DMG for either 1 or 3 mol % of PEG200-stearic acid (PEG-SA). All particles showed a slightly negative zeta potential at pH 7.4, and a zeta potential between 12.5 and 23 mV at pH 5.

TABLE 30
mCherry expression in human dendritic
cells following incubation at 1 μg/ml

| LNP formulation | MFI |
|---|---|
| UT | 1,343 ± 263 |
| KC3-OA, 5% DSPS | 74,561 ± 8,663 |
| SM102 | 4,242 ± 1,942 |
| ALC-0315 | 3,760 ± 1,625 |
| 45 mol % KC3-OA, 5% DSPS (N/P = 5) | 70,437 ± 26,182 |
| 45 mol % KC3-OA, 5% DSPS (N/P = 5.5) | 71,757 ± 25,238 |
| 45 mol % KC3-OA, 5% DSPS (N/P = 6) | 29,810 ± 11,956 |
| 45 mol % KC3-OA, 5% DSPS (N/P = 6.5) | 17,921 ± 8,100 |
| 43% KC3-OA, 5% DSPS (N/P = 5) | 39,229 ± 13,743 |
| 48% KC3-OA, 5% DSPS (N/P = 5) | 117,973 ± 42,550 |
| 45% KC3-OA, 5% DSPS, 1% PEG-SA (N/P = 5) | 76,331 ± 26,839 |
| 45% KC3-OA, 5% DSPS, 3% PEG-SA (N/P = 5) | 38,667 ± 21,077 |
| 45% KC3-OA, 5% DSPS, 1% PEG-SA (N/P = 6) | 74,019 ± 46,480 |
| 45% KC3-OA, 5% DSPS, 3% PEG-SA (N/P = 6) | 49,934 ± 24,738 |

TABLE 31
mCherry expression in human dendritic
cells following incubation at 0.1 μg/ml

| LNP formulation | MFI |
|---|---|
| UT | 1,295 ± 116 |
| KC3-OA, 5% DSPS | 5,670 ± 886 |
| SM102 | 1,492 ± 26 |
| ALC-0315 | 1,405 ± 28 |
| 45 mol % KC3-OA, 5% DSPS (N/P = 5) | 10,035 ± 2,773 |
| 45 mol % KC3-OA, 5% DSPS (N/P = 5.5) | 11,461 ± 3,826 |
| 45 mol % KC3-OA, 5% DSPS (N/P = 6) | 9,014 ± 1,673 |
| 45 mol % KC3-OA, 5% DSPS (N/P = 6.5) | 5,214 ± 1,678 |
| 43% KC3-OA, 5% DSPS (N/P = 5) | 8,403 ± 2,185 |
| 48% KC3-OA, 5% DSPS (N/P = 5) | 13,434 ± 5,292 |
| 45% KC3-OA, 5% DSPS, 1% PEG-SA (N/P = 5) | 7,943 ± 2,848 |
| 45% KC3-OA, 5% DSPS, 3% PEG-SA (N/P = 5) | 6,362 ± 1,502 |
| 45% KC3-OA, 5% DSPS, 1% PEG-SA (N/P = 6) | 7,197 ± 1,495 |
| 45% KC3-OA, 5% DSPS, 3% PEG-SA (N/P = 6) | 6,601 ± 1,756 |

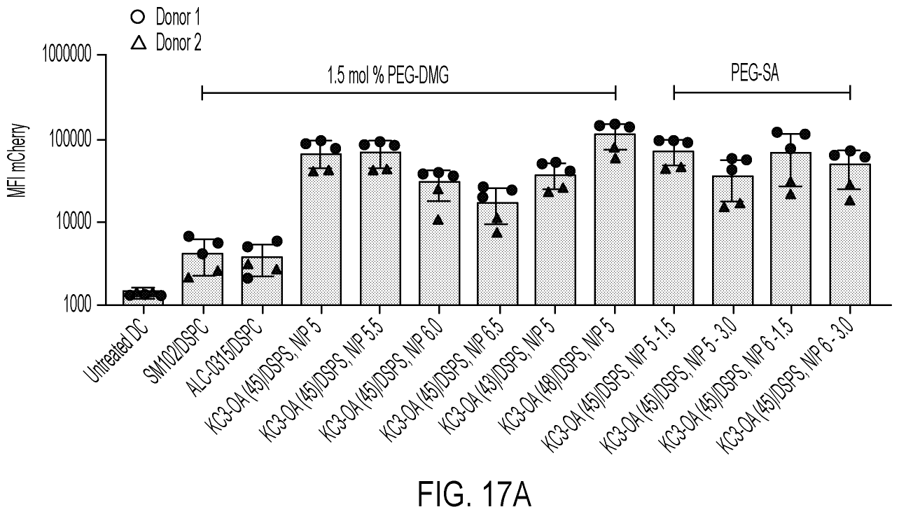

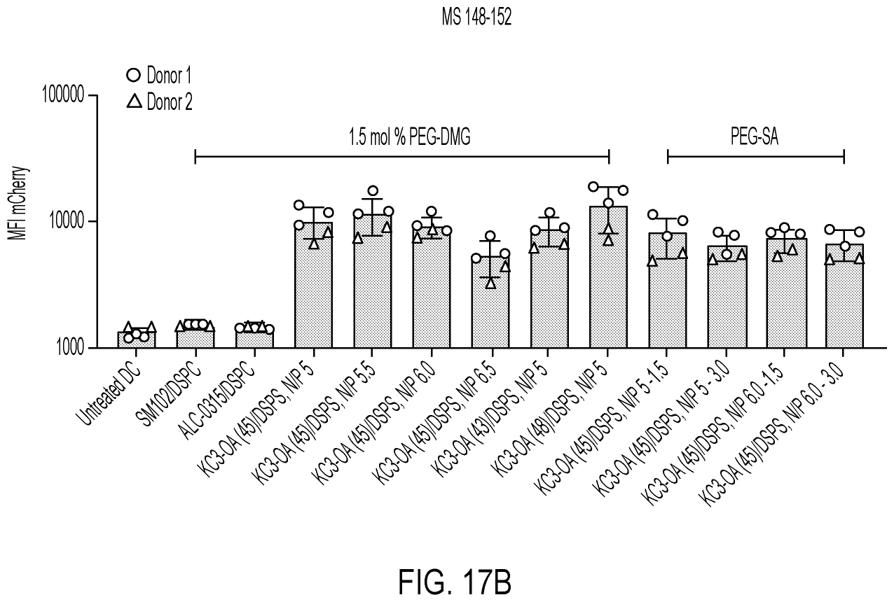

The effect of targeting and ICL choice on transfection activity was evaluated using 5 mol % DSPS-targeted LNPs comprised of KC3-OA at concentrations ranging from 43-48 mol % (FIGS. 17A-17B) and N/P from 5-6.5. LNP formulations at N/P 5 and 6, and 45 mol % KC3-OA were also prepared with PEG-SA at both 1 and 3 mol % and compared to similar formulation with 1.5 mol % PEG-DMG. All formulations were highly active, although formulations with PEG-DMG did display about 30-40% higher transfection than those with PEG-SA. The formulations did trend over the range to have higher transfection going from 43 to 48 mol % KC3-OA. All LNP variations of this DSPS-targeted KC3-OA LNP formulation were significantly more active than either SM-102/DSPC or ALC-0315/DSPC controls at both concentrations evaluated (Tables 42 and 43).

### Example 26. Targeting of Human Dendritic Cells with Phosphatidylglycerol

The aim of this study was to explore the effect of targeting LNPs containing UO-1 or KC3-01 ICLs with various densities of distearoylphosphatidylglycerol (DSPG) on transfection efficiency in human dendritic cells. DSPG was incorporated in the LNPs at densities ranging from 0-10 mol % of the total lipid content. Control LNPs included KC3-OA LNPs with the previously established 5 mol % of DSPS as the targeting lipid, and nontargeted SM-102 or ALC-0315 containing LNPs. LNPs were prepared as described in Example 2, characterized for particle size and zeta potential as described in Example 3, and evaluated for transfection efficiency in human dendritic cells as described in Example 16. The LNPs used the ionizable lipids in Table 45 in the ICL with a constant N/P ratio of 5.25 and 48 mol %, the DSPG (Na⁺ salt) lipid was included at 0-10 mol % and the DSPC phospholipid constant at 10 mol %-mol % incorporated of DSPG (total of DSPG and DSPC was 10 mol %), and the cholesterol constant at 40.5 mol %. PEG-DMG was held constant in all formulations at 1.5 mol %. The final ALC-0315/DSPC formulation was composed of 46.3 mol % ALC-0315, 10 mol % DSPC, 42.7 mol % cholesterol and 1.5 mol % PEG-DMG. The SM-102/DSPC formulation was composed of 50 mol % SM102, 10 mol % DSPC, 38.5 mol % cholesterol and 1.5 mol % PEG-DMG.

TABLE 32
Physicochemical properties and characterization of LNPs used in Example 26

| | | Zeta | Zeta | |
|---|---|---|---|---|
| | Particle | Potential | Potential | Encapsulation |
| LNP formulation | size (nm) | (mV) pH 5 | (mV) pH 7 | Efficiency (%) |
| KC3-OA, 5% DSPS | 85.2 | 13.9 | −1.9 | 90.3 ± 6.4 |
| SM102 | 71.0 | 6.0 | −3.5 | 84.1 ± 10.7 |
| ALC-0315 | 63.0 | 5.2 | −5.2 | 89.0 ± 5.9 |
| UO1 | 99.2 | 25.4 | 2.9 | 86.9 ± 2.5 |
| UO1, 1.25% DSPG | 101.2 | 17.7 | 0.4 | 85.3 ± 2.4 |
| UO1, 2.5% DSPG | 89.7 | 13.5 | −2.3 | 83.8 ± 8.4 |
| UO1, 5.0% DSPG | 81.2 | 17.3 | −0.7 | 90.9 ± 2.7 |
| UO1, 7.5% DSPG | 65.9 | 15.9 | −1.8 | 91.0 ± 8.1 |
| UO1, 10% DSPG | 67.7 | 16.7 | −2.9 | 90.5 ± 6.6 |
| KC3-01 | 95.0 | 26.4 | 1.5 | 90.3 ± 11.5 |
| KC3-01, 1.25% DSPG | 98.4 | 27.4 | 0.0 | 90.7 ± 3.1 |
| KC3-01, 2.5% DSPG | 82.1 | 18.5 | −0.8 | 90.6 ± 3.2 |
| KC3-01, 5.0% DSPG | 72.4 | 18.1 | −2.1 | 89.9 ± 3.8 |
| KC3-01, 7.5% DSPG | 71.0 | 14.8 | −2.2 | 89.4 ± 3.6 |
| KC3-01, 10% DSPG | 74.6 | 13.0 | −2.3 | 89.5 ± 13.5 |

TABLE 33
mCherry expression in human dendritic
cells following incubation at 1 μg/ml

| | | | vs ALC- | vs SM- |
|---|---|---|---|---|
| | | | 0315 | 102 |
| | | | LNP | LNP |
| LNP formulation | MFI | T/NT | (fold) | (fold) |
| Untreated (UT) | 1,453 ± 263 | | | |
| KC3-OA, 5% DSPS | 89,815 ± 16,603 | | 76.8 | 8.4 |
| SM102 | 11,924 ± 1,089 | | | |
| ALC-0315 | 2,604 ± 376 | | | |
| UO1 | 2,049 ± 297 | | 0.5 | 0.1 |
| UO1, 1.25% DSPG | 2,728 ± 950 | 2.1 | 1.1 | 0.1 |
| UO1, 2.5% DSPG | 8,134 ± 3,763 | 11.2 | 5.8 | 0.6 |
| UO1, 5.0% DSPG | 22,206 ± 3,488 | 34.8 | 18.0 | 2.0 |
| UO1, 7.5% DSPG | 12,424 ± 1,939 | 18.4 | 9.5 | 1.0 |
| UO1, 10% DSPG | 5,561 ± 639 | 6.9 | 3.6 | 0.4 |
| KC3-01 | 49,931 ± 14,344 | | 42.1 | 4.6 |
| KC3-01, 1.25% DSPG | 119,467 ± 21,513 | 2.4 | 102.6 | 11.3 |
| KC3-01, 2.5% DSPG | 112,191 ± 12,156 | 2.3 | 96.2 | 10.6 |
| KC3-01, 5.0% DSPG | 86,696 ± 10,231 | 1.8 | 74.1 | 8.1 |
| KC3-01, 7.5% DSPG | 25,230 ± 2,061 | 0.5 | 20.7 | 2.3 |
| KC3-01, 10% DSPG | 8,250 ± 2,442 | 0.1 | 5.9 | 0.6 |

TABLE 34
mCherry expression in human dendritic
cells following incubation at 0.1 μg/ml

| | | | vs ALC- | vs SM- |
|---|---|---|---|---|
| | | | 0315 | 102 |
| | | T/NT | LNP | LNP |
| LNP formulation | MFI | (fold) | (fold) | (fold) |
| Untreated (UT) | 1,403 ± 190 | | | |
| KC3-OA, 5% DSPS | 10,347 ± 3,118 | | 308.6 | 48.0 |
| SM102 | 1,590 ± 243 | | | |
| ALC-0315 | 1,432 ± 198 | | | |
| UO1 | 1,393 ± 237 | | | |
| UO1, 1.25% DSPG | 1,409 ± 262 | 0.5 | 0.2 | 0 |
| UO1, 2.5% DSPG | 1,474 ± 280 | 6.7 | 2.4 | 0.4 |
| UO1, 5.0% DSPG | 2,253 ± 1,089 | 81.0 | 29.3 | 4.6 |
| UO1, 7.5% DSPG | 1,887 ± 400 | 46.2 | 16.7 | 2.6 |
| UO1, 10% DSPG | 1,515 ± 252 | 10.7 | 3.9 | 0.6 |
| KC3-01 | 1,976 ± 112 | | 19.8 | 3.1 |
| KC3-01, 1.25% DSPG | 2,377 ± 329 | 1.7 | 33.6 | 5.2 |
| KC3-01, 2.5% DSPG | 3,801 ± 485 | 4.2 | 82.7 | 12.9 |
| KC3-01, 5.0% DSPG | 8,580 ± 786 | 12.5 | 247.6 | 38.5 |
| KC3-01, 7.5% DSPG | 3,075 ± 279 | 2.9 | 57.7 | 9.0 |
| KC3-01, 10% DSPG | 1,619 ± 146 | 0.4 | 7.4 | 1.2 |

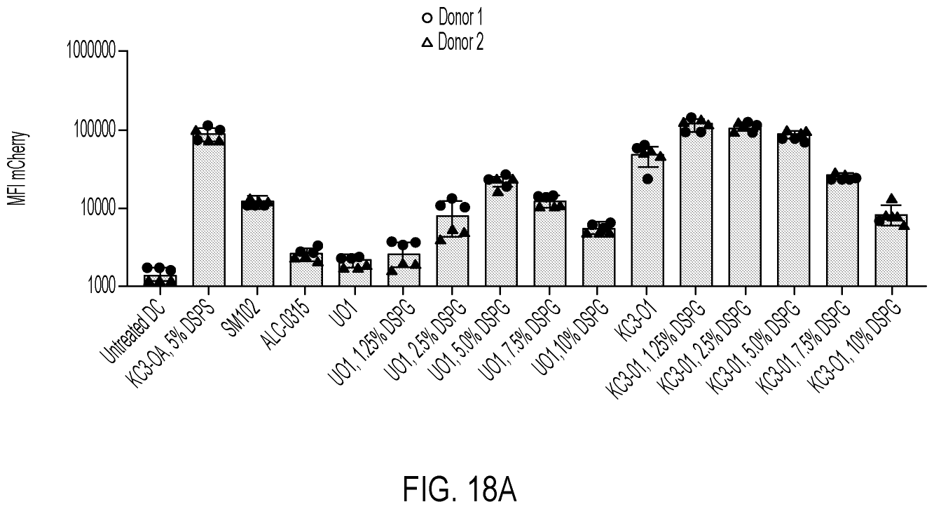

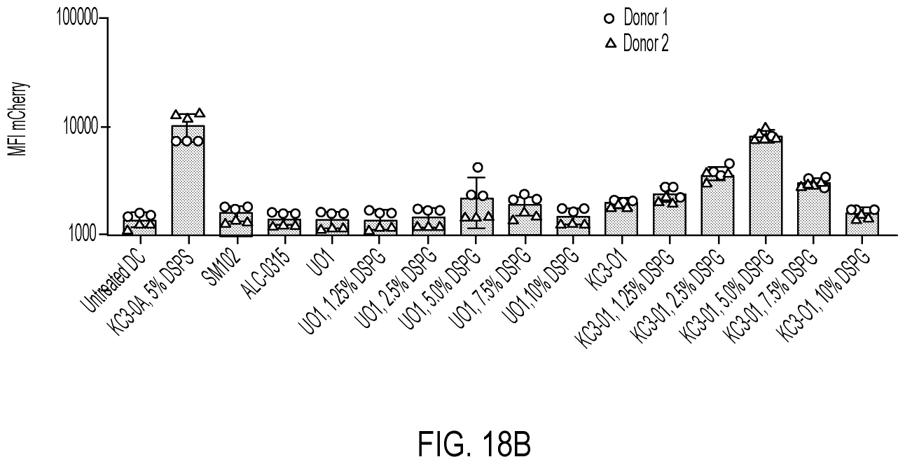

The effect of targeting two different LNPs with DSPG on transfection activity was evaluated using 0-10 mol % DSPG-targeted LNPs (FIGS. 18A-18B). For UO-1 containing LNPs the optimum DSPG concentration was 5 mol % at both 1 and 0.1 ug/ml, resulting in a 34.8 folding targeting effect at 1 ug/ml and 81-fold improvement over nontargeted LNPs at 0.1 ug/ml. For KC3-01 containing LNPs the optimum DSPG concentration was also at 5 mol % for the 0.1 ug/mL, but showed a broader peak of 1.25-5 mol % at the higher concentration of 1 ug/mL with a targeting effect of 2.4-fold at 1 ug/mL and 12.5-fold at the lower concentration of 0.1 ug/mL. Both of the peak targeted LNPs showed increased transfection activity compared to nontargeted ALC-0315 and SM-102 LNPs (Tables 46 and 47), with an up to 102.6% increase compared to ALC-0315 for DSPG-targeted KC3-01 LNPs at 1 ug/mL and an up to 247.6 fold increase at 0.1 ug/mL. The improvement over SM-102 LNPs was as high as 38.50-fold for the 5 mol % DSPG targeted KC3-01 LNP and 48-fold for the DSPS-targeted targeted KC3-OA LNP control. This data shows that DSPG can be a potent targeting anionic lipid to increase transfection efficiency in human dendritic cells.

### Example 27. Comparison of DSPC and DPPC as Helper Lipids in a KC3-PA-Based LNP Formulation

The aim of this study was to explore comparing DSPC and DPPC in KC3-PA DPPS-targeted and non-targeted LNPs. The concentration of KC3-OA was 48 mol %, cholesterol 40.5 mol %, PEG-DMG 1.5 mol %. The phospholipid composition was kept constant at 10 mol %, but for those that were targeted with DPPS, the DPPS concentration was 5 mol % and either DSPC or DPPC at 5 mol %. All formulations had an N/P of 5.25.

LNPs were prepared as described in Example 2, and were characterized for particle size, mRNA concentration and % mRNA entrapment after ethanol removal by dialysis and after sterile filtration as described in Example 3.

TABLE 35
Physicochemical properties and characterization
of KC3-PA-targeted and non-targeted LNPs

| | Particle | | |
|---|---|---|---|
| | Size (nm) | | % mRNA |
| | after | Encapsulation | Loss upon |
| LNP formulation | filtration | Efficiency (%) | filtration |
| KC3-PA/DPPC | 108.5 | 82.2 ± 2.4 | 35.5 |
| KC3-PA/DPPC/DPPS-NH⁴ | 107.7 | 90.6 ± 6.1 | 10.6 |
| KC3-PA/DSPC | 114.0 | 86.4 ± 5.7 | 12.5 |
| KC3-PA/DSPC/DPPS-NH⁴ | 91.6 | 91.3 ± 6.7 | 4.4 |

Once the LNPs have been prepared and the ethanol removed by dialysis, the measurement of mRNA concentration before and after filtration provides another metric to evaluate the formulations. It is advantageous if LNPs have a high efficiency of sterile filtration, where mRNA loss is minimized and eliminates membrane fouling and poor product yields. In the non-targeted group, we found a 2.8-fold improvement in mRNA recovery with the DSPC sample compared to DPPC and in the targeted group we found a 2.4-fold increase in mRNA recovery. This suggests that DSPC is a more compatible helper lipid in formulations with KC3-PA than DPPC with and without 5 mol % DPPS.

### Example 28. Impact of Ionizable Lipid and ICL Density on Immunogenicity of SARS-COV-2 Spike Protein mRNA LNPs

LNPs were prepared as described in Example 2, characterized for particle size and zeta potential as described in Example 4, and evaluated for transfection efficiency in human dendritic cells as described in Example 16. The LNPs used the ionizable lipids in Table 36 in the ICL with a constant N/P ratio of 5.25. The concentration of ICL used in a particular formulation is displayed in the graph. For example, 46.5% KC3-OA %/5% DSPC/5% DSPS formulation contains, 46.5 mol % KC3-OA, 5 mol % DSPC, 5 mol % DSPS, 42 mol % cholesterol and 1.5 mol % PEG-DMG, or 1575 nmol KC3-OA, 169.4 nmol DSPC, 169.4 nmol DSPS, 1422.6 nmol cholesterol and 50.8 nmol PEG-DMG per 100 μg mRNA. The PEG-DMG concentration was held constant at 1.5 mol % for all samples. If the ICL concentration is increased, there is a proportional decrease in the cholesterol concentration. For formulations with KC-like ICLs, they were targeted with 5 mol % DSPS and for UO-like ICLs, they were targeted with 7.5 mol % DSPS.

The ALC-0315 formulation was composed of 46.3 mol % ALC-0315, 10 mol % DSPC, 42.7 mol % cholesterol and 1.56 mol % PEG-DMG.

TABLE 36
Physicochemical properties and characterization of
LNPs containing either ALC-0315, KC2, KC3-OA, UO-1
and UO-9 containing SARS-CoV-2 spike protein mRNA

| | | Zeta | Zeta | |
|---|---|---|---|---|
| | Particle | Potential | Potential | Encapsulation |
| LNP | Size | (mV) | (mV) | Efficiency |
| formulation | (nm) | pH 5 | pH 7 | (%) |
| ALC-0315 | 99.2 | 25.4 | 2.9 | 81.1 ± 3.7 |
| 46.5% KC2/5% | 89.7 | 13.5 | −2.3 | 92.1 ± 3.8 |
| DSPC/5% DSPS | | | | |
| 46.5% KC3-01/5% | 81.2 | 17.3 | −0.7 | 92.3 ± 6.4 |
| DSPC/5% DSPS | | | | |
| 46.5% KC3-OA/5% | 65.9 | 15.9 | −1.8 | 91.3 ± 7.7 |
| DSPC/5% DSPS | | | | |
| 48% KC3-OA/5% | 67.8 | 16.7 | −2.9 | 89.9 ± 7.2 |
| DSPC/5% DSPS | | | | |
| 46.5 KC3-OA/5% | 95.0 | 26.4 | 1.5 | 91.5 ± 3.8 |
| DPPC/5% DSPS | | | | |
| 48% UO-1/5% | 82.1 | 18.5 | −0.8 | 91.9 ± 7.8 |
| DSPC/7.5% DSPS | | | | |
| 48% UO-9/5% | 72.4 | 18.1 | −2.1 | 92.8 ± 6.5 |
| DSPC/7.5% DSPS | | | | |

However, it was not clear if lowering the mol % of the ionizable lipid would negatively impact in vivo immunogenicity. Using the same methods outlined in Example 25, BALB/c mice were immunized intramuscularly with 1 μg of vaccine candidates and blood was collected 21 days later.

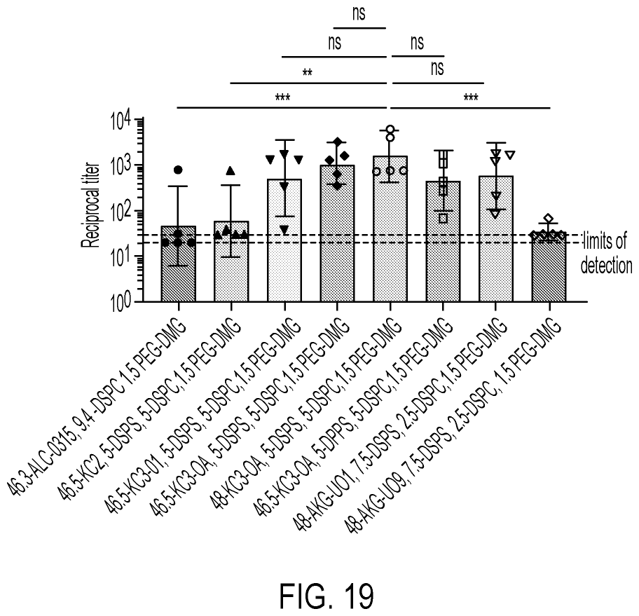

Serum was assayed for total anti-spike IgG antibodies by ELISA. The geometric mean titers were similar between mice immunized with LNPs containing either 46.5 or 48 mol % KC3-OA. LNPs containing 46.5 mol % of the ionizable lipid KC3-01 were similarly immunogenic (FIG. 19). All KC3-01 or KC3-OA LNPs showed significantly higher titers when compared to ALC-0315 or KC2 LNP controls.

Mice were also immunized with vaccine candidates containing the ionizable lipids AKG-UO1 and AKG-UO9. AKG-UO1 induced antibody titers similar to KC3-OA, but LNPs containing AKG-UO9 were poorly immunogenic.

### Example 29. Impact of PG and PS Targeting of KC3-OA Containing LNPs on mRNA Expression in Human Dendritic Cells

The aim of this study was to explore the effect of targeting LNPs containing KC3-OA ICLs with various forms of phosphatidylglycerol (PG), including distearoylphosphatidylglycerol (DSPG), dipalmitoylphosphatidylglycerol (DPPG), dioleoylphosphatidylglycerol (DOPG), and dimyristoylphosphatidylglycerol (DMPG) on transfection efficiency in human dendritic cells. A second aim was to compare these targeted LNPs to those containing DSPS, and finally to compare formulations that incorporated two anionic phospholipids (DSPG and DSPS).

PG in various forms was incorporated in the LNPs at densities ranging from 0-5 mol % of the total lipid content. Control LNPs included KC3-OA LNPs with the previously established 5 mol % of DSPS as the targeting lipid. LNPs were prepared as described in Example 2, characterized for particle size and zeta potential as described in Example 3, and evaluated for transfection efficiency in human dendritic cells as described in Example 16. The LNPs used the ionizable lipids in Table 52 in the ICL with a constant N/P ratio of 5.25 and 48 mol %, the DSPG (Na⁺ salt) lipid was included at 0-10 mol % and the DSPC phospholipid was also varied from 0-10 mol %, and the cholesterol constant at 40.5 mol %. Some formulations included DSPS and DSPG, with or without DSPC. PEG-DMG was held constant in all formulations at 1.5 mol %.

TABLE 37
Physicochemical properties and characterization
of LNPs used in Example

| | | Zeta | Zeta | |
|---|---|---|---|---|
| | Particle | Potential | Potential | Encapsulation |
| LNP | size | (mV) | (mV) | Efficiency |
| formulation | (nm) | pH 5 | pH 7 | (%) |
| 10% DSPC | 91.9 | 22.19 | 1.712 | 92.7 ± 3.1 |
| 5% DSPG/5% DSPC | 94.0 | 15.01 | −6.99 | 90.3 ± 2.1 |
| 5% DOPG/5% DSPC | 77.0 | 15.64 | 2.303 | 92.0 ± 7.5 |
| 5% DPPG/5% DSPC | 93.6 | 14.7 | −0.2016 | 90.6 ± 2.7 |
| 5% DMPG/5% DSPC | 66.0 | 19.29 | 0.8961 | 92.8 ± 8.0 |
| 5% DSPS/5% DSPC | 87.9 | 23.18 | −1.219 | 92.7 ± 4.5 |
| 5% DSPG/5% DSPS | 69.9 | 25.61 | −1.651 | 93.3 ± 6.1 |
| 2.5% DSPG/5% | 72.2 | 19.36 | −1.596 | 92.7 ± 6.0 |
| DSPS/2.5% DSPC | | | | |
| 5% DSPG/5% | 66.6 | 16.72 | −5.128 | 92.6 ± 2.0 |
| DSPS/2.5% DSPC | | | | |
| 5% DSPG/5% | 69.1 | 17.75 | −4.506 | 93.5 ± 3.3 |
| DSPS/5%DSPC | | | | |
| 5% DSPG/5% | 66.9 | 11.11 | −5.389 | 93.4 ± 9.0 |
| DSPS/7.5% DSPC | | | | |
| 5% DSPG/5% | 64.6 | 15.43 | −6.708 | 93.2 ± 4.2 |
| DSPS/10% DSPC | | | | |

All formulations showed particle sizes between 65-100 nm with a slightly negative charge at pH 7.4 and a clearly positive zeta potential of 10-25 mV at pH 5. All particles had greater than 90% encapsulation efficiency, despite the presence of anionic phospholipids that could compete for binding with the negative charged phosphates on mRNA.

TABLE 38
mCherry expression in human dendritic cells following incubation at 1 μg/ml

| | MFI | T/NT | MFI | T/NT |
|---|---|---|---|---|
| LNP formulation | (donor 1) | (donor 1) | (donor 2) | (donor 2) |
| Untreated (UT) | 2,735 ± 56 | | 1,354 ± 19 | |
| 10% DSPC | 14,152 ± 661 | | 14,034 ± 5,257 | |
| 5% DSPG/5% DSPC | 43,413 ± 5,029 | 3.56 | 53,670 ± 1,974 | 4.13 |
| 5% DOPG/5% DSPC | 16,783 ± 1,375 | 1.23 | 62,404 ± 2,841 | 4.81 |
| 5% DPPG/5% DSPC | 39,042 ± 6,957 | 3.18 | 58,557 ± 3,456 | 4.51 |
| 5% DMPG/5% DSPC | 70,516 ± 1,584 | 1.79 | 51,862 ± 5,953 | 3.98 |
| 5% DSPS/5% DSPC | 70,516 ± 1,584 | 5.94 | 225,546 ± 11,751 | 17.68 |
| 5% DSPG/5% DSPS | 10,148 ± 313 | 0.65 | 15,158 ± 289 | 1.09 |
| 2.5% DSPG/5% | 35,797 ± 2,364 | 2.90 | 85,066 ± 8,576 | 6.60 |
| DSPS/2.5% DSPC | | | | |
| 5% DSPG/5% | 27,349 ± 4,520 | 2.16 | 59,403 ± 4,237 | 4.58 |
| DSPS/2.5% DSPC | | | | |
| 5% DSPG/5% | 52,500 ± 1,351 | 4.36 | 98,242 ± 1,421 | 7.64 |
| DSPS/5% DSPC | | | | |
| 5% DSPG/5% | 49,439 ± 3,660 | 4.09 | 80,055 ± 7,036 | 6.21 |
| DSPS/7.5% DSPC | | | | |
| 5% DSPG/5% | 32,566 ± 4,525 | 2.61 | 47,190 ± 9,456 | 3.61 |
| DSPS/10% DSPC | | | | |

TABLE 39
mCherry expression in human dendritic cells following incubation at 0.1 μg/ml

| | MFI | T/NT | MFI | T/NT |
|---|---|---|---|---|
| LNP formulation | (donor 1) | (donor 1) | (donor 2) | (donor 2) |
| Untreated (UT) | 2,687 ± 27 | | 1,390 ± 11 | |
| 10% DSPC | 3,330 ± 216 | | 1,721 ± 36 | |
| 5% DSPG/5% DSPC | 6,292 ± 155 | 5.61 | 4,289 ± 19 | 8.76 |
| 5% DOPG/5% DSPC | 4,288 ± 52 | 2.49 | 5,951 ± 370 | 13.78 |
| 5% DPPG/5% DSPC | 6,754 ± 312 | 6.32 | 5,243 ± 535 | 11.64 |
| 5% DMPG/5% DSPC | 4,912 ± 647 | 3.46 | 4,202 ± 199 | 8.49 |
| 5% DSPS/5% DSPC | 10,776 ± 161 | 12.58 | 21,283 ± 1,303 | 60.10 |
| 5% DSPG/5% DSPS | 3,379 ± 112 | 1.08 | 3,574 ± 178 | 6.60 |
| 2.5% DSPG/5% | 6,190 ± 184 | 5.45 | 11,379 ± 133 | 30.18 |
| DSPS/2.5% DSPC | | | | |
| 5% DSPG/5% | 4,967 ± 291 | 3.55 | 6,028 ± 65 | 14.01 |
| DSPS/2.5% DSPC | | | | |
| 5% DSPG/5% | 8,353 ± 153 | 8.81 | 9,758 ± 214 | 25.28 |
| DSPS/5% DSPC | | | | |
| 5% DSPG/5% | 10,604 ± 126 | 12.31 | 10,602 ± 1,620 | 27.83 |
| DSPS/7.5% DSPC | | | | |
| 5% DSPG/5% | 11,761 ± 502 | 14.11 | 10,139 ± 339 | 26.43 |
| DSPS/10% DSPC | | | | |

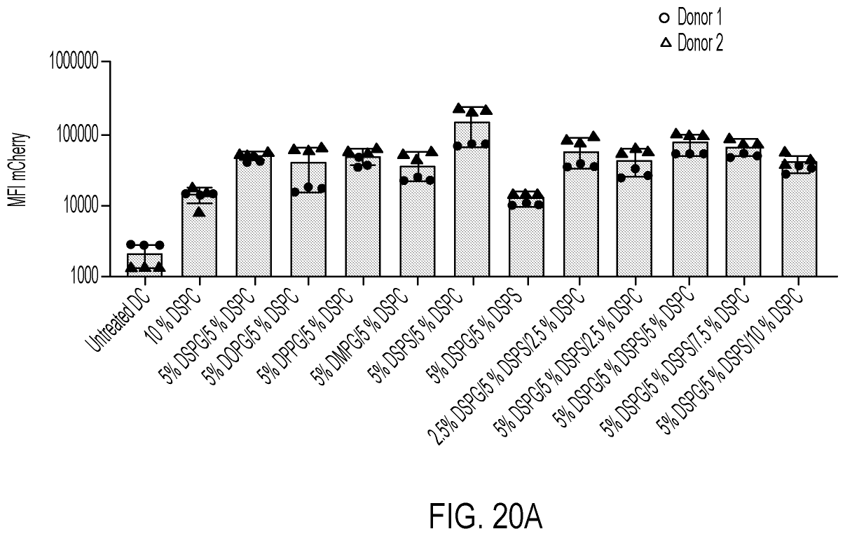

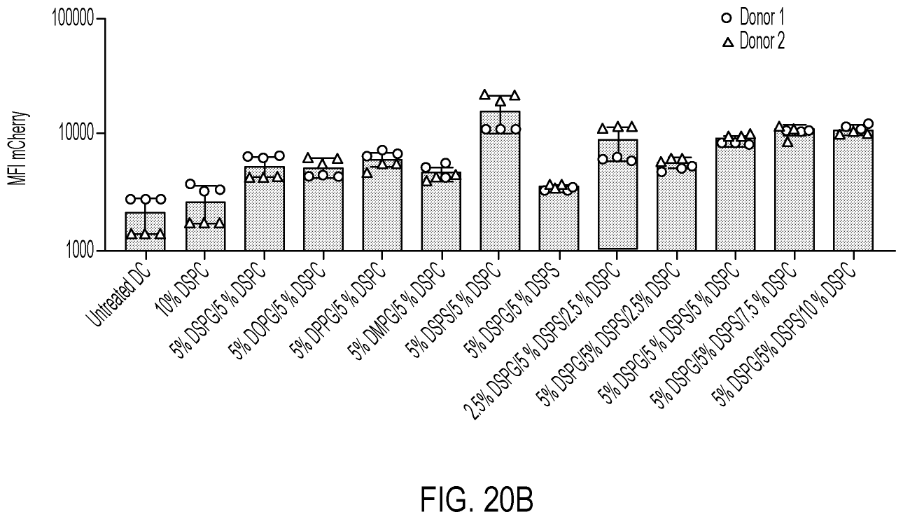

The effect of targeting two different LNPs with DSPG on transfection activity was evaluated using PG-targeted LNPs of various compositions (FIGS. 20A-20B). For KC3-OA containing LNPs, all forms of PG showed some level of increased expression relatively to 10% DSPC controls, and although there was small preference for the saturated DPPG or DSPG forms of PG with one donor, in a separate donor DOPG and DMPG showed similar activity. The highest increase over DSPC control came from the 5% DSPS targeted LNP, and combining DSPS with DSPG did not improve transfection activity further, and in most instances was antagonistic when compared to those formulations containing DSPS as the sole anionic phospholipid. In fact, those LNP formulations where all the phospholipids were either DSPG or DSPS (e.g. no DSPC) showed the lowest transfection activity of the targeted LNP formulations. This data shows that both PG and DSPS can be potent targeting anionic lipids to increase transfection efficiency in human dendritic cells, although combining the two does not appear to further improve activity.

### Example 30. Use of Ethanol Soluble DPPS-NH4 as Opposed to the Less Soluble DSPS-Na Allows for Room Temperature Preparation of Phosphatidylserine-Targeted KC3OA-Based LNPs

The aim of this study was to compare DSPS-Na and DPPS-NH4 as targeting ligands in LNPs by measuring their transfection efficiency in murine DC cells. LNPs were prepared as in Example 3. Prior to LNP preparation stock solutions of each lipid were made in ethanol before use, except DSPS-Na, which because of its low solubility in ethanol (see Example 27) was dissolved in methanol at elevated temperatures (70° C.) to ensure complete dissolution. Once cooled to room temperature, the methanol solution will maintain DSPS-Na solubility for a short period of time (<1 hour). Therefore, after addition of KC3OA, DSPC, Chol and PEG-DMG and DSPS-Na from their respective lipid stocks, the resulting mixture contains about 16% methanol by volume in ethanol. To make DSPS-targeted LNPs, and to ensure complete solubility of DSPS-Na, the ethanol/methanol solution is incubated at 70 C in a syringe holder heating block prior to mixing with pre-warmed mRNA. Care must be taken to maintain elevated temperature control prior to LNP formation, otherwise DSPS aggregation or LNP aggregation can occur. Once the LNPs were formed, the suspension was allowed to cool naturally to room temperature before solvent removal by dialysis. KC3OA was kept constant at 48 mol %, the DSPC concentration was 5 mol % with either 5 mol % DSPS-Na or DPPS-NH₄, 38.5 mol % cholesterol and 1.5 mol % PEG-DMG. The N/P was 5.25. The KC3OA/DSPS sample contained 1575 nmol KC3OA, 164.1 nmol DSPC, 164.1 nmol DSPS-Na, 1328.9 nmol cholesterol and 49.2 nmol PEG-DMG per 100 μg mRNA. In contrast, because of the higher solubility of DPPS-NH₄, LNPs can be prepared from all ethanol stock solutions, thereby eliminating the need for high temperature incubation before mixing and eliminates methanol from the process, which are both advantageous from a scale-up and residual solvent perspective. The KC3OA/DPPS sample contained 1575 nmol KC3OA, 164.1 nmol DSPC, 164.1 nmol DPPS-NH₄, 1328.9 nmol cholesterol and 49.2 nmol PEG-DMG per 100 μg mRNA and was mixed at room temperature. The final ALC-0315/DSPC formulation was composed of 46.3 mol % ALC-0315, 10 mol % DSPC, 42.7 mol % cholesterol and 1.5 mol % PEG-DMG. The SM-102/DSPC formulation was composed of 50 mol % SM102, 10 mol % DSPC, 38.5 mol % cholesterol and 1.5 mol % PEG-DMG, both controls prepared at room temperature.

LNPs were exchanged into PBS, pH 7.4 and then 15 mM Tris, pH 7.4, 20% sucrose, concentrated to 20-50 ug/mL mRNA using an Amicon-Ultra 4 (100,000 MWCO) spin column, sterile filtered (Thermo Nalgene 0.2 um #720-1320) prior to freezing by immersion in liquid nitrogen for 5 min and long-term storage at −80° C. LNPs were analyzed as described in Example 3, and were tested in murine DC cells as described in Example 4.

TABLE 40

| | | Zeta | Zeta | |
|---|---|---|---|---|
| | Particle | Potential | Potential | Encapsulation |
| LNP | Size | (mV) | (mV) | Efficiency |
| formulation | (nm) | pH 5 | pH 7 | (%) |
| ALC-0315 | 71.0 | 6.0 | −3.5 | 84.1 ± 10.7 |
| SM102 | 63.0 | 5.2 | −5.2 | 89.0 ± 5.9 |
| KC3-OA/DSPS-Na | 83.9 | 19.04 | −0.8 | 92.6 ± 8.7 |
| KC3-OA/DPPS-NH₄ | 78.3 | 11.91 | −0.1 | 93.3 ± 1.2 |

TABLE 41

| | | vs | vs |
|---|---|---|---|
| | | ALC-0315 | SM-102 |
| | mCherry | LNP | LNP |
| LNP formulation | MFI | (fold) | (fold) |
| Untreated (UT) | 282.7 ± 3.8 | | |
| ALC-0315 | 1336.3 ± 91.6 | | |
| SM-102 | 6978.3 ± 1388 | | |
| KC3-OA/DSPS-Na | 16039.3 ± 4046.4 | 12.0 | 2.3 |
| KC3-OA/DPPS- NH₄ | 32326.3 ± 735.9 | 24.2 | 4.6 |

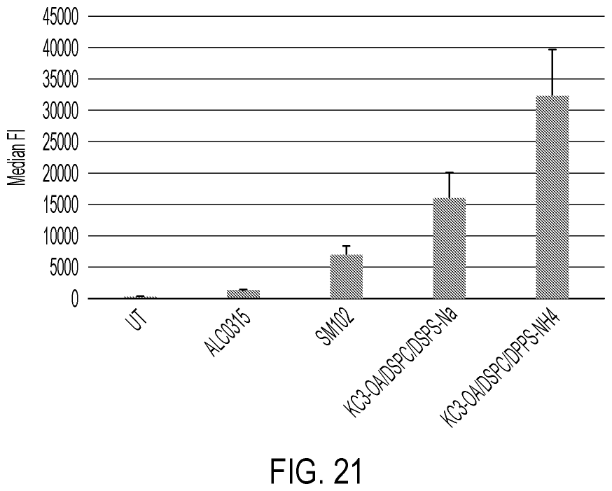

The KC3-OA/DPPS-NH₄ LNP was comparable to the DSPS-targeted LNP from a biophysical point of view, all measured characteristics were very similar. In addition, it was found that inclusion of DPPS induced an approximately 2-fold increase in mCherry expression (FIG. 21). The comparable biophysical LNP characteristics combined with the comparable and slightly improved mRNA expression, suggests that DPPS-NH₄ is an attractive PS-targeting ligand targeting for KC3-OA LNPs.

### Example 31: Synthesis of Cationic Lipids with Asymmetric Chains and Also where One Chain is Saturated Alkyl and Another is Monounsaturated, C15 or C17

### Synthesis of (7Z, 24Z)-tritriaconta-7,24-dien-16-one (asymmetric C₁₅(8:1)-C₁₇(8:1) ketone)

An equimolar mixture of oleoyl chloride and palmitoleoyl chloride is processed essentially as described above for the synthesis of (9Z,26Z)-pentatriaconta-9,26-dien-18-one (2). The products (C₁₅(8:1)-C₁₅(8:1), C₁₇(8:1)-C₁₇(8:1), and C₁₅(8:1)-C₁₇(8:1) ketones) are separated using column chromatography to isolate the asymmetric C₁₅(8:1)-C₁₇(8:1) ketone. The structure is confirmed by NMR.

### Synthesis of 3-(S)-2-(8Z)-pentadec-8-en-1-yl-2′-(8Z)-heptadec-8-en-1-yl-1,3-dioxolan-4-yl)-N,N-dimethylpropan-1-amine

The procedure for the synthesis of 3-((S)-2,2-di((Z)-heptadec-8-en-1-yl)-1,3-dioxolan-4-yl)-N,N-dimethylpropan-1-amine, AKG-KC3-C17(C8:1)), described above, is essentially followed using (7Z, 24Z)-tritriaconta-7,24-dien-16-one as a starting material. A cationic lipid having general structure I-A with one C₁₇ monounsaturated and one C₁₅ monounsaturated R₁ hydrocarbon chain is obtained.

### Synthesis of (9Z)-pentatriacont-9-en-18-one (asymmetric C₁₇(8:1)-C₁₇ ketone)

An equimolar mixture of oleoyl chloride and stearoyl chloride is processed essentially as described above for the synthesis of (9Z,26Z)-pentatriaconta-9,26-dien-18-one (2). The products (C₁₇(8:1)-C₁₇(8:1), C₁₇(8:1)-C₁₇, and C₁₇-C₁₇ketones) are separated using column chromatography to isolate the asymmetric C₁₇(8:1)-C₁₇ ketone. The structure is confirmed by NMR.

### Synthesis of 3-(S)-2-(8Z)-heptadec-8-en-1-yl-2′-heptadec-1-yl-1,3-dioxolan-4-yl)-N,N-dimethylpropan-1-amine

The procedure for the synthesis of 3-((S)-2,2-di((Z)-heptadec-8-en-1-yl)-1,3-dioxolan-4-yl)-N,N-dimethylpropan-1-amine, AKG-KC3-C17(C8:1)), described above, is essentially followed using (9Z)-pentatriacont-9-en-18-one as a starting material. A cationic lipid having general structure I-A with one C₁₇ monounsaturated and one C₁₇ saturated (alkyl) R₁ hydrocarbon chain is obtained.

### Synthesis of (24Z)-tritriacont-24-en-16-one (asymmetric C₁₅-C₁₇(8:1) ketone)

An equimolar mixture of oleoyl chloride and palmitoyl chloride is processed essentially as described above for the synthesis of (9Z,26Z)-pentatriaconta-9,26-dien-18-one (2). The products (C₁₇(8:1)-C₁₇(8:1), C₁₅-C₁₅, and C₁₅-C₁₇(8:1) ketones) are separated using column chromatography to isolate the asymmetric C₁₅-C₁₇(8:1) ketone. The structure is confirmed by NMR.

### Synthesis of 3-(S)-2-(8Z)-pentadec-8-en-1-yl-2′-heptadec-1-yl-1,3-dioxolan-4-yl)-N,N-dimethylpropan-1-amine

The procedure for the synthesis of 3-((S)-2,2-di((Z)-heptadec-8-en-1-yl)-1,3-dioxolan-4-yl)-N,N-dimethylpropan-1-amine, AKG-KC3-C17(C8:1)), described above, is essentially followed using (24Z)-tritriacont-24-en-16-one as a starting material. A cationic lipid having general structure I-A with one C₁₇ monounsaturated and one C₁₅ saturated (alkyl) R₁ hydrocarbon chain is obtained.

### Example 32: Preparation of Mtb mRNA MHC Class II Vaccine Cassettes

Background Tuberculosis (TB) is one of the leading causes of death worldwide, and it is estimated that a quarter of the global population is infected with the causative microbe, Mycobacterium tuberculosis (Mtb) (WHO Global Tuberculosis Report 2021). A primary method of disease prevention is childhood immunization with bacilli Calmette-Guerin (BCG) vaccine, which is the only approved TB vaccine. BCG has been in existence for over 100 years, and while infant immunization can protect against severe forms of disseminated forms of childhood disease, vaccine protection wanes in adolescence and adulthood and it provides variable to no protection against development of active TB disease. Consequently, there is an urgent need to develop a new vaccine that either works in conjunction with or replaces BCG in order to meet the WHO's End TB Strategy milestones.

T cells are critical for controlling Mtb infection and preventing active disease. CD4 T cells in particular have a dominant role. Mice depleted of CD4 T cells are highly susceptible to infection and disease, and disruption of T helper 1 (Th1) cytokines such as IFN-γ and TNF-α, and the transcription factor Tbet increases susceptibility (Urdahl (2014) Semin Immunol 26, 578-587; Caruso et al. (1999) J. Immunol 162, 5407-5416). Humans with deficiencies in IFN-γ and the Th1 cytokine IL-12 are also more susceptible (source). CD4 T cell depletion in non-human primate models of infection also increases susceptibility to infection, and depletion in latently infected macaques greatly increases the chance reactivation (Urdahl (2014) Semin Immunol 26, 578-587; Flynn et al. (2015) Immunol Rev 264, 60-73). In humans, coinfected people living with HIV and latent TB infection have a 20-30-fold increased lifetime risk of developing active TB disease and experience worse outcomes (Esmail et al. (2018) Annu Rev Immunol 36, 603-638). CD4 T cells are the primary cell type infected by HIV, and while peripheral blood absolute CD4 T cell counts do not necessary correlate with increased risk of TB disease, HIV infection results in decreased Mtb-specific CD4 T cells circulating in the blood, and phenotypic/functional changes in the CD4 T cell compartment correlate with increased risk of developing active disease (Esmail et al. (2018) Annu Rev Immunol 36, 603-638). CD8 T cells also contribute to controlling Mtb infection. Mtb-specific CD8 T cells recognizing a broad array of proteins are present in the circulation, pulmonary lymph nodes and lungs of latently infected people (Lin and Flynn (2015) Semin Immunopathol 37, 239-249; Lewinsohn et al. (2013) PLoS One 8, e67016; Commandeur et al. (2011 Clin Vaccine Immunol 18, 676-683; Kamath et al. (2004) J Exp Med 200, 1479-1484), are important in controlling bacterial burden (Woodworth et al. (2008) J Immunol 181, 8595-8603; Pinxteren et al. (2000) Eur J Immunol 30, 3689-3698), and contribute to immune protection after BCG immunization (Chen (2009) PLoS Pathog 5, e1000392).

Given the importance of both CD4 and CD8 T cells in protection against TB, there has been significant effort to identify T cell antigen specificities in humans. Much more is known about the Mtb-specific CD4 T cell repertoire than for CD8 T cells in latently infected humans. Lindestam Arlehamn, Sette and colleagues conducted unbiased genome-wide approaches using an HLA class II epitope prediction algorithm coupled with high-throughput screening of peptide libraries. Initially using a cohort of healthy latently infected (LTBI) individuals in San Diego, California, they identified a large number of antigen-specificities that, at a population level, were largely focused on antigenic “islands” of proteins (Arlehamn et al. (2013) PLoS Pathog 9, e1003130; Arlehamn et al. (2016) PLoS Pathog 12, e1005760). Importantly, the individual breadth of CD4 T cell responses within these antigenic islands was broad with an average of twenty-four specificities identified per person, but substantial variation existed between individuals (Arlehamn et al. (2013) PLoS Pathog 9, e1003130). Building on these findings, CD4 T cell specificities from a South African LTBI cohort to vaccine candidate antigens and IFN-γ release assay (IGRA) antigens were examined (Arlehamn et al. (2016) PLoS Pathog 12, e1005760). From these two studies, pools of epitopes were identified that captured the majority of circulating Mtb-specific IFNg-producing CD4 T cells. A comprehensive list of 300 epitopes was defined (MTB300), a subset of 125 epitopes (MTB125) was recognized by at least one South African individual, and a core of 66 unique immunodominant epitopes (MTB66) recognized by >1 individual was able to capture ~80% of the peripheral CD4 T cell response at a population level.

Relative to CD4 T cells, fewer minimal CD8 T cell epitopes have been identified. Using a short list of Mtb proteins known to be immunogenic to CD4 T cells, limited dilution cloning of CD8 T cells from LTBI donors was used to identify class I restricted epitopes (Lewinsohn et al. (2007) PLoS Pathog 3, 1240-1249). This approach yielded a small number of defined epitopes primarily restricted to HLA-B alleles. Tang et al. used a combination of epitope prediction algorithms and experimental filters to identify a larger list of minimal epitopes restricted to the HLA-A2, HLA-A3 and HLA-B7 superfamilies, with the most focus placed on characterizing HLA-A2-restricted epitopes (Tang et al. (2011) J Immunol 186, 1068-1080). While these three HLA class I superfamilies are predicted to capture much of the global population, there will likely be significant population gaps with most HLA alleles falling into nine supertypes (Sidney et al. (2008) BMC Immunol 9:1). More recently, Lewinsohn et al. used overlapping peptide libraries covering 389 Mtb proteins predicted to be immunogenic to characterize CD8 T cell responses in diverse LTBI individuals (Lewinsohn et al. (2017) NPJ Vaccines 2, 8). While minimal HLA class I epitopes were not identified within the libraries, at the protein level secreted proteins were enriched for immunodominant responses, and there was substantial overlap in proteins immunogenic to both CD4 and CD8 T cells.

Due to the abundance of characterized CD4 T cell epitopes, the well-studied binding promiscuity of epitopes between different HLA class II alleles (Greenbaum et al. (2011) Immunogenetics 63, 325-335), and the finding that epitopes are focused within antigenic islands, we reasoned that a CD4 T cell-focused mRNA vaccine could be designed from the epitopes in the MTB66, MTB125 and MTB300 pools. However, antigen selection for a CD8 T cell-focused mRNA vaccine is perhaps more challenging. Relative to CD4 T cells, there are fewer defined CD8 T cell epitopes with known HLA I restrictions, and while defined epitopes have been identified for some HLA class I supertypes, these are likely insufficient to provide global vaccine coverage. Thus we used an unbiased approach to identify a set of class I epitopes predicted to provide global coverage.

Selection of antigens for a CD4 T cell-focused TB vaccine. To capture the broadest repertoire of immunodominant CD4 T cell epitopes identified in healthy LTBI people, the MTB300, MTB 125 and MTB66 epitope lists defined by Lindestam Arlehamn et al. (Arlehamn et al. (2016) PLoS Pathog 12, e1005760) were compiled, sorted by gene (Rv number) and annotated to identify the peptide location within the ORF reference sequence. Peptides that were overlapping or adjacent were combined. Peptides then fell into two categories, those that were densely clustered and fell into hot spots, and those that were non-overlapping and located in ORFs with fewer identified epitopes. The densely clustered “hotspot” ORFs are listed in Table 42 and altogether they capture 92 epitopes from the MTB300 list and 36 of the 66 most immunogenic epitopes included in the MTB66 list.

To capture additional immunodominant epitopes and increase HLA coverage, the algorithm PopCover-2.0 was used to prioritize epitope selection of the remaining non-overlapping epitopes (Nilsson et al. Front (2021) Immunol 12, 728936; services.healthtech.dtu.dk/service.php?PopCover-2.0). PopCover-2.0 reduces an input dataset of predicted epitopes by removing sequence redundancies and then selecting a user-defined number of epitopes that achieve the broadest population HLA allele coverage. First, non-overlapping epitopes were input into the binding prediction algorithm “IEDB recommended 2.22” in the Immune Epitope Binding Database (accessed through IEDB.com) and predictions were made using the “7 allele” reference set of HLA class II alleles described by Paul et al. (Paul et al. (2015) J Immunol Methods 422, 28-34). Only predicted epitopes and their HLA II restrictions with a percentile rank <20 were kept. The following were then input into the PopCover-2.0 algorithm: 1) the predicted epitopes and their HLA restrictions, 2) the complete Mtb ORF protein sequences corresponding to the epitopes, and 3) the 7 allele reference set and their corresponding population frequencies (table 2 in Paul et al. (2015) J Immunol Methods 422, 28-34). The number of epitopes to select was set to 10 and the length of peptides to extract from the ORF protein sequences was set to 15 amino acids. The PopCover-2.0 output of the top 10 epitopes is listed in Table 43 as SEQ ID NOs 8-15 and are predicted to provide 34.5% coverage to individuals with the HLA-DRB1 locus, 51.4% coverage to the HLA-DRB3 locus, 41.8% coverage to the HLA-DRB4 locus, 16.0% coverage to the HLA-DRB5 locus, and 84.4% coverage across all HLA class II loci. By adding these additional 10 15mer epitopes to the hot spot ORFs, 46 out of 66 MTB66 epitopes are captured.

A cassette consisting of a string of the selected antigens can then be assembled from the described proteins. To prevent irrelevant junctional neoantigens from forming between two juxtaposed proteins, a GPGPG spacer (SEQ ID NO: 228) was inserted (Livingston et al., (2002) J Immunol 168, 5499-5506). The starting methionine residue was removed from internal ORFs as it is not necessary. One example of a concatenated cassette is shown in Table 44 and SEQ ID NO. 18. Another example of a concatenated cassette is shown in Table 45 and SEQ ID NO. 19. Another example of a concatenated cassette is shown in Table 46 and SEQ ID NO. 20.

Use of signal sequences in a CD4 T cell-based mRNA vaccine cassette. Signal peptides/sequences direct nascently translated proteins from the cytoplasm to cellular compartments. These can be engineered into the vaccine cassette to direct vaccine antigens to cellular compartments that will support the type of protein processing optimal for antigen delivery to immune cells. For sample, B cells recognize proteins in their native form, and these can be extracellular soluble proteins or, in the case of nucleotide-based vaccines that are translated in situ, cell-membrane associated. As CD4 and CD8 T cells recognize their antigen in the context of short peptides bound to MHC class II and I, respectively, proteins must be processed by the appropriate cellular machinery. For class II, proteins are processed in the endosomal compartment and loaded onto class II molecules that are then shuttled to the cell surface for presentation to CD4 T cells. For class I, cytoplasmic proteins are cleaved by the proteosome, shuttled into the endoplasmic reticulum (ER) where they bind to class I molecules, and these complexes are then shuttled to the cell surface for presentation to CD8 T cells. These are the canonical pathways of antigen processing and presentation, but there are alternative pathways. For instance, class I molecules contain a signal sequence that can direct them to endosomes, and antigens that have been processed to the appropriate length can be loaded there and transported to the cell surface (Kreiter et al., (2008) J Immunol 180, 309-318).

Signal peptides (SP) have been widely used to in the production of synthetic proteins and they have been engineered into nucleotide-based vaccines to improve antigen presentation to B and T cells. In the case of nucleotide vaccines, cytoplasmic proteins are generally poor at priming CD4 T cells. To improve MHC class II presentation, signal sequences that direct protein antigens to the endosomal/lysosomal compartment have been used. One example is the use of the N- and C-terminal SPs from lysosome-associated membrane protein 1 (LAMP-1). The N-terminal SP (SEQ ID NO. 21) directs nascent proteins to the ER lumen and the C-terminal transmembrane and cytoplasmic domains (SEQ ID NO. 22) directs proteins to the late endosome/lysosome (Wu et al. (1995) Proc Natl Acad Sci USA 92, 11671-11675; Bonini et al. (2001) J Immunol 166, 5250-5257). Interestingly, vaccine antigens directed to the endo/lysosome using LAMP-1 SPs was found to supported class I presentation and priming to CD8 T cells (Bonini et al. (2001) J Immunol 166, 5250-5257). Another example of endo/lysosomal-targeting signal sequences that support antigen presentation to both CD4 and CD8 T cells is the MHC class I N- and C-terminal signal sequences (Lizee et al. (2003) Nat Immunol 4, 1065-1073; Kreiter et al., (2008) J Immunol 180, 309-318). Even though peptide loading onto MHC class I canonically occurs in the ER before shuttling to the plasma membrane, class I proteins contain a transmembrane and cytoplasmic domain that directs class I to endosomal and lysosomal compartments in a pattern that largely overlaps with class II molecules (Lizee et al. (2003) Nat Immunol 4, 1065-1073; Kreiter et al., (2008) J Immunol 180, 309-318). Two variants of the N-terminal HLA class I SP (also called sec domain) are listed in SEQ ID NO. 23 and SEQ ID NO. 24. Two variants of the C-terminal transmembrane/cytoplasmic domains are listed in SEQ ID NO. 25 and SEQ ID NO. 26. A third approach used in nucleotide-based vaccines is to use the class II HLA-DRα signal sequence to co-localize nascent proteins with class II molecules in the endo/lysosomal compartment (SEQ ID NO. 27, SEQ ID NO. 28, SEQ ID NO. 29). A fourth approach is to use a secretion signal peptide to promote extracellular release of soluble vaccine antigen followed by cellular uptake and entry of a vaccine antigen through the endosomal pathway. One such SP has been adapted from the tissue-type plasminogen activator (tPA) protein (Li et al. (1999) Infect Immun 67, 4780-4786; Wang et al. (2011) Appl Microbiol Biotechnol 91, 731-740). The tPA SP increases secretion of target proteins, and a mutated version with a P22A mutation was found to further enhance protein secretion (Wang et al. (2011) Appl Microbiol Biotechnol 91, 731-740) (SEQ ID NO. 30).

Integration of antigenic proteins, signal peptides and UTRs into a CD4 T cell-focused mRNA vaccine. A vaccine cassette is assembled by combining the antigenic protein, the desired SP, flanking 5′ and 3′ untranslated regions (UTRs) and an optimized Kozak sequence (GCCACC). One set of UTRs is from the human hemoglobin subunit beta protein (SEQ ID NO. 32). For instance, one example of a cassette is SEQ ID NO. 33 that consists of the LAMP1 N-terminal SP (SEQ ID NO. 21) and C-terminal transmembrane/cytoplasmic domains (SEQ ID NO. 22) and the Mtb-derived antigenic protein listed in SEQ ID NO. 18; the associated forward codon-optimized nucleotide sequence with HBB UTRs (SEQ ID NO. 32) is SEQ ID NO. 34. A second example is SEQ ID NO. 35 that consists of the same HBB UTRs and LAMP1 SPs but uses a different Mtb-derived antigenic protein sequence shown in Table 45 and SEQ ID NO. 19; the associated forward codon optimized nucleotide sequence with HBB UTRs is SEQ ID NO. 36. A third example is SEQ ID NO. 37 that uses a different Mtb-derived antigenic protein sequence (SEQ ID NO. 20 and Table 46) and the sec/MITD signal sequences; the associated forward codon optimized nucleotide sequence with HBB UTRs is SEQ ID 38. A fourth example is SEQ ID NO. 39 that uses the same antigenic protein sequence as before (SEQ ID NO. 20) but with LAMP-1 signal sequences; the associated forward codon optimized nucleotide sequence with HBB UTRs is SEQ ID NO. 40. A fifth example is SEQ ID NO. 41 that uses the same antigen protein sequence (SEQ ID NO. 20) but with the human HLA-DRα chain signal peptide; the associated forward codon optimized nucleotide sequence with HBB UTRs is SEQ ID NO. 42. A sixth example is SEQ ID NO. 43 that uses the same antigen protein sequence (SEQ ID NO. 20) but with the tPA SP; the associated forward codon optimized nucleotide sequence with HBB UTRs is SEQ ID NO. 44.

TABLE 42
Open reading frames (ORFs) that are densely clustered
with CD4 T cell epitopes. Protein IDs are accession
numbers (www.ncbi.nlm.nih.gov/protein)

| | | antigenic hotspots used | |
|---|---|---|---|
| gene | protein | in mRNA constructs | Protein IDs |
| Rv0288 | esxH, TB10.4 | 2-96 | NP_214802.1 |
| Rv1886c | fbpB, Ag85b | 111-200 | NP_216402 |
| Rv1196 | PPE18, Mtb39a | 2-249 | YP_177795.1 |
| Rv3619c | esxV | 2-94 | NP_218136.1 |
| Rv3620c | esxW | 2-98 | NP_218137.1 |
| Rv3874 | esxB, CFP10 | 2-100 | NP_218391.1 |
| Rv3875 | esxA, ESAT-6 | 2-95 | YP_178023.1 |

TABLE 43
List of epitopes not in antigen hot spots that were incorporated
into a CD4 T cell-focused mRNA vaccine. Protein IDs are accession
numbers.(www.ncbi.nlm.nih.gov/protein).

| | | | amino | | |
|---|---|---|---|---|---|
| | | | acids | | Protein |
| SEQ ID | gene | protein | used | sequence | IDs |
| SEQ ID NO. 8 | Rv1788 | PE18 | 1-15 | MSFVTTQPEALAAAA | YP_177834.1 |
| SEQ ID NO. 9 | Rv1788 | PE18 | 59-73 | AQIYQAVSAQAAAIH | YP_177834.1 |
| SEQ ID NO. 10 | Rv3804c | fbpA, | 57-71 | PSPSMGRDIKVQFQS | NP_218321.1 |
| | | Ag85a | | | |
| SEQ ID NO. 11 | Rv1387 | PPE20 | 129-144 | GINTIPIAINEAEYV | YP_177806.1 |
| SEQ ID NO. 12 | Rv3020c | esxS | 49-64 | AAFQGAHARFVAAAA | YP_177919.1 |
| SEQ ID NO. 13 | Rv1047 | Rv1047 | 210-224 | AGWLAFFRDLVARGL | NP_215563.1 |
| SEQ ID NO. 14 | Rv1047 | Rv1047 | 360-374 | ASIIRLVGAVLAEQH | NP_215563.1 |
| SEQ ID NO. 15 | Rv1195 | PE13 | 1-15 | MHVSFVMAYPEMLAA | ALA77657.1, |
| | | | | | CKM34396.1 |
| SEQ ID NO. 16 | Rv2031c | hspX | 94-108 | AYGSFVRTVSLPVGA | NP_216547.1 |
| SEQ ID NO. 17 | Rv3330 | dacB1 | 265-279 | LENDNQLLYNYPGAL | NP_217847.1 |

TABLE 44
One possible combination of Mtb antigens to create a vaccine
cassette. The table corresponds to SEQ ID NO. 18.

| Order in | | amino | |
|---|---|---|---|
| cassette | gene/protein | acids | sequence |
| 1 | Rv3874/esxB | 2-100 | AEMKTDAATLAQEAGNFERISGDLKTQIDQVES |
| | | | TAGSLQGQWRGAAGTAAQAAVVRFQEAANKQKQ |
| | | | ELDEISTNIRQAGVQYSRADEEQQQALSSQMGF |
| | | | (SEQ ID NO. 6) |
| 2 | Rv3875/csxA | 2-95 | TEQQWNFAGIEAAASAIQGNVTSIHSLLDEGKQ |
| | | | SLTKLAAAWGGSGSEAYQGVQQKWDATATELNN |
| | | | ALQNLARTISEAGQAMASTEGNVTGMFA (SEQ |
| | | | ID NO. 7) |
| 3 | Rv0288/esxH | 2-96 | SQIMYNYPAMLGHAGDMAGYAGTLQSLGAEIAV |
| | | | EQAALQSAWQGDTGITYQAWQAQWNQAMEDLVR |
| | | | AYHAMSSTHEANTMAMMARDTAEAAKWGG |
| | | | (SEQ ID NO. 1) |
| 4 | Rv1886c/Ag85b | 111-200 | PVGGQSSFYSDWYSPACGKAGCQTYKWETFLTS |
| | | | ELPQWLSANRAVKPTGSAAIGLSMAGSSAMILA |
| | | | AYHPQQFIYAGSLSALLDPSQGMG (SEQ ID |
| | | | NO. 2) |
| 5 | Rv1196/PPE18 | 2-249 | VDFGALPPEINSARMYAGPGSASLVAAAQMWDS |
| | | | VASDLFSAASAFQSVVWGLTVGSWIGSSAGLMV |
| | | | AAASPYVAWMSVTAGQAELTAAQVRVAAAAYET |
| | | | AYGLTVPPPVIAENRAELMILIATNLLGQNTPA |
| | | | IAVNEAEYGEMWAQDAAAMFGYAAATATATATL |
| | | | LPFEEAPEMTSAGGLLEQAAAVEEASDTAAANQ |
| | | | LMNNVPQALQQLAQPTQGTTPSSKLGGLWKTVS |
| | | | PHRSPISNMVSMANNHM (SEQ ID NO. 3) |
| 6 | Rv3620c/esxW | 2-98 | TSRFMTDPHAMRDMAGRFEVHAQTVEDEARRMW |
| | | | ASAQNISGAGWSGMAEATSLDTMTQMNQAFRNI |
| | | | VNMLHGVRDGLVRDANNYEQQEQASQQILSS |
| | | | (SEQ ID NO. 5) |
| 7 | Rv3619c/esxV | 2-94 | TINYQFGDVDAHGAMIRAQAGSLEAEHQAIISD |
| | | | VLTASDFWGGAGSAACQGFITQLGRNFQVIYEQ |
| | | | ANAHGQKVQAAGNNMAQTDSAVGSSWA (SEQ |
| | | | ID NO. 4) |
| 8 | Rv1788/PE18 | 59-73 | AQIYQAVSAQAAAIH (SEQ ID NO. 9) |
| 9 | Rv3804c/Ag85a | 57-71 | PSPSMGRDIKVQFQS (SEQ ID NO. 10) |
| 10 | Rv1387/PPE20 | 129-144 | GINTIPIAINEAEYV (SEQ ID NO. 11) |
| 11 | Rv3020c/esxS | 49-64 | AAFQGAHARFVAAAA (SEQ ID NO. 12) |
| 12 | Rv1047/Rv1047 | 210-224 | AGWLAFFRDLVARGL (SEQ ID NO. 13) |
| 13 | Rv1047/Rv1047 | 360-374 | ASIIRLVGAVLAEQH (SEQ ID NO. 14) |
| 14 | Rv1788/PE18 | 1-15 | MSFVTTQPEALAAAA (SEQ ID NO. 8) |
| 15 | Rv1195/PE13 | 1-15 | MHVSFVMAYPEMLAA (SEQ ID NO. 15) |
| 16 | Rv20631c/hspX | 94-108 | AYGSFVRTVSLPVGA (SEQ ID NO. 16) |
| 17 | Rv3330/dacB1 | 265-279 | LENDNQLLYNYPGAL (SEQ ID NO. 17) |

TABLE 45
One possible combination of Mtb antigens to create a vaccine
cassette. The table corresponds to SEQ ID NO. 19.

| Order in | | amino | |
|---|---|---|---|
| cassette | gene/protein | acids | sequence |
| 1 | Rv3874/esxB | 2-100 | AEMKTDAATLAQEAGNFERISGDLKTQIDQVES |
| | | | TAGSLQGQWRGAAGTAAQAAVVRFQEAANKQKQ |
| | | | ELDEISTNIRQAGVQYSRADEEQQQALSSQMGF |
| | | | (SEQ ID NO. 6) |
| 2 | Rv3875/csxA | 2-95 | TEQQWNFAGIEAAASAIQGNVTSIHSLLDEGKQ |
| | | | SLTKLAAAWGGSGSEAYQGVQQKWDATATELNN |
| | | | ALQNLARTISEAGQAMASTEGNVTGMFA (SEQ |
| | | | ID NO. 7) |
| 3 | Rv3620c/esxW | 2-98 | TSRFMTDPHAMRDMAGRFEVHAQTVEDEARRMW |
| | | | ASAQNISGAGWSGMAEATSLDTMTQMNQAFRNI |
| | | | VNMLHGVRDGLVRDANNYEQQEQASQQILSS |
| | | | (SEQ ID NO. 5) |
| 4 | Rv3619c/esxV | 2-94 | TINYQFGDVDAHGAMIRAQAGSLEAEHQAIISD |
| | | | VLTASDFWGGAGSAACQGFITQLGRNFQVIYEQ |
| | | | ANAHGQKVQAAGNNMAQTDSAVGSSWA (SEQ |
| | | | ID NO. 4) |
| 5 | Rv0288/esxH | 2-96 | SQIMYNYPAMLGHAGDMAGYAGTLQSLGAEIAV |
| | | | EQAALQSAWQGDTGITYQAWQAQWNQAMEDLVR |
| | | | AYHAMSSTHEANTMAMMARDTAEAAKWGG |
| | | | (SEQ ID NO. 1) |
| 6 | Rv1886c/Ag85b | 111-200 | PVGGQSSFYSDWYSPACGKAGCQTYKWETFLTS |
| | | | ELPQWLSANRAVKPTGSAAIGLSMAGSSAMILA |
| | | | AYHPQQFIYAGSLSALLDPSQGMG (SEQ ID |
| | | | NO. 2) |
| 7 | Rv1196/PPE18 | 2-249 | VDFGALPPEINSARMYAGPGSASLVAAAQMWDS |
| | | | VASDLFSAASAFQSVVWGLTVGSWIGSSAGLMV |
| | | | AAASPYVAWMSVTAGQAELTAAQVRVAAAAYET |
| | | | AYGLTVPPPVIAENRAELMILIATNLLGQNTPA |
| | | | IAVNEAEYGEMWAQDAAAMFGYAAATATATATL |
| | | | LPFEEAPEMTSAGGLLEQAAAVEEASDTAAANQ |
| | | | LMNNVPQALQQLAQPTQGTTPSSKLGGLWKTVS |
| | | | PHRSPISNMVSMANNHM (SEQ ID NO. 3) |
| 8 | Rv1788/PE18 | 59-73 | AQIYQAVSAQAAAIH (SEQ ID NO. 9) |
| 9 | Rv3804c/Ag85a | 57-71 | PSPSMGRDIKVQFQS (SEQ ID NO. 10) |
| 10 | Rv1387/PPE20 | 129-144 | GINTIPIAINEAEYV (SEQ ID NO. 11) |
| 11 | Rv3020c/esxS | 49-64 | AAFQGAHARFVAAAA (SEQ ID NO. 12) |
| 12 | Rv1047/Rv1047 | 210-224 | AGWLAFFRDLVARGL (SEQ ID NO. 13) |
| 13 | Rv1047/Rv1047 | 360-374 | ASIIRLVGAVLAEQH (SEQ ID NO. 14) |
| 14 | Rv1788/PE18 | 1-15 | MSFVTTQPEALAAAA (SEQ ID NO. 8) |
| 15 | Rv1195/PE13 | 1-15 | MHVSFVMAYPEMLAA (SEQ ID NO. 15) |
| 16 | Rv20631c/hspX | 94-108 | AYGSFVRTVSLPVGA (SEQ ID NO. 16) |
| 17 | Rv3330/dacB1 | 265-279 | LENDNQLLYNYPGAL (SEQ ID NO. 17) |

TABLE 46
One possible combination of Mtb antigens to create a vaccine
cassette. The table corresponds to SEQ ID NO. 20.

| Order in | | amino | |
|---|---|---|---|
| cassette | gene/protein | acids | sequence |
| 1 | Rv3874/esxB | 2-100 | AEMKTDAATLAQEAGNFERISGDLKTQIDQVES |
| | | | TAGSLQGQWRGAAGTAAQAAVVRFQEAANKQKQ |
| | | | ELDEISTNIRQAGVQYSRADEEQQQALSSQMGF |
| | | | (SEQ ID NO. 6) |
| 2 | Rv1196/PPE18 | 2-249 | VDFGALPPEINSARMYAGPGSASLVAAAQMWDS |
| | | | VASDLFSAASAFQSVVWGLTVGSWIGSSAGLMV |
| | | | AAASPYVAWMSVTAGQAELTAAQVRVAAAAYET |
| | | | AYGLTVPPPVIAENRAELMILIATNLLGQNTPA |
| | | | IAVNEAEYGEMWAQDAAAMFGYAAATATATATL |
| | | | LPFEEAPEMTSAGGLLEQAAAVEEASDTAAANQ |
| | | | LMNNVPQALQQLAQPTQGTTPSSKLGGLWKTVS |
| | | | PHRSPISNMVSMANNHM (SEQ ID NO. 3) |
| 3 | Rv3875/esxA | 2-95 | TEQQWNFAGIEAAASAIQGNVTSIHSLLDEGKQ |
| | | | SLTKLAAAWGGSGSEAYQGVQQKWDATATELNN |
| | | | ALQNLARTISEAGQAMASTEGNVTGMFA (SEQ |
| | | | ID NO. 7) |
| 4 | Rv3620c/esxW | 2-98 | TSRFMTDPHAMRDMAGRFEVHAQTVEDEARRMW |
| | | | ASAQNISGAGWSGMAEATSLDTMTQMNQAFRNI |
| | | | VNMLHGVRDGLVRDANNYEQQEQASQQILSS |
| | | | (SEQ ID NO. 5) |
| 5 | Rv0288/esxH | 2-96 | SQIMYNYPAMLGHAGDMAGYAGTLQSLGAEIAV |
| | | | EQAALQSAWQGDTGITYQAWQAQWNQAMEDLVR |
| | | | AYHAMSSTHEANTMAMMARDTAEAAKWGG |
| | | | (SEQ ID NO. 1) |
| 6 | Rv3619c/esxV | 2-94 | TINYQFGDVDAHGAMIRAQAGSLEAEHQAIISD |
| | | | VLTASDFWGGAGSAACQGFITQLGRNFQVIYEQ |
| | | | ANAHGQKVQAAGNNMAQTDSAVGSSWA (SEQ |
| | | | ID NO. 4) |
| 7 | Rv1886c/Ag85b | 111-200 | PVGGQSSFYSDWYSPACGKAGCQTYKWETFLTS |
| | | | ELPQWLSANRAVKPTGSAAIGLSMAGSSAMILA |
| | | | AYHPQQFIYAGSLSALLDPSQGMG (SEQ ID |
| | | | NO. 2) |
| 8 | Rv1788/PE18 | 59-73 | AQIYQAVSAQAAAIH (SEQ ID NO. 9) |
| 9 | Rv3804c/Ag85a | 57-71 | PSPSMGRDIKVQFQS (SEQ ID NO. 10) |
| 10 | Rv1387/PPE20 | 129-144 | GINTIPIAINEAEYV (SEQ ID NO. 11) |
| 11 | Rv3020c/esxS | 49-64 | AAFQGAHARFVAAAA (SEQ ID NO. 12) |
| 12 | Rv1047/Rv1047 | 210-224 | AGWLAFFRDLVARGL (SEQ ID NO. 13) |
| 13 | Rv1047/Rv1047 | 360-374 | ASIIRLVGAVLAEQH (SEQ ID NO. 14) |
| 14 | Rv1788/PE18 | 1-15 | MSFVTTQPEALAAAA (SEQ ID NO. 8) |
| 15 | Rv1195/PE13 | 1-15 | MHVSFVMAYPEMLAA (SEQ ID NO. 15) |
| 16 | Rv20631c/hspX | 94-108 | AYGSFVRTVSLPVGA (SEQ ID NO. 16) |
| 17 | Rv3330/dacB1 | 265-279 | LENDNQLLYNYPGAL (SEQ ID NO. 17) |

### Sequence ID NOs.

Mycobacterium tuberculosis H37Rv|Rv0288|esxH|TB10.4.
Amino acids 2-96
SEQ ID NO. 1
SQIMYNYPAMLGHAGDMAGYAGTLQSLGAEIAVEQAALQSAWQGDTGITYQAWQAQWNQAMED
LVRAYHAMSSTHEANTMAMMARDTAEAAKWGG.
Mycobacterium tuberculosis H37Rv|Rv1886c|fbpB|Ag85b.
Amino acids 111-200
SEQ ID NO. 2
PVGGQSSFYSDWYSPACGKAGCQTYKWETFLTSELPQWLSANRAVKPTGSAAIGLSMAGSSAM
ILAAYHPQQFIYAGSLSALLDPSQGMG.
Mycobacterium tuberculosis H37Rv|Rv1196|PPE18|Mtb39a.
Amino acids 2-249.
SEQ ID NO. 3
VDFGALPPEINSARMYAGPGSASLVAAAQMWDSVASDLFSAASAFQSVVWGLTVGSWIGSSAG
LMVAAASPYVAWMSVTAGQAELTAAQVRVAAAAYETAYGLTVPPPVIAENRAELMILIATNLL
GQNTPAIAVNEAEYGEMWAQDAAAMFGYAAATATATATLLPFEEAPEMTSAGGLLEQAAAVEE
ASDTAAANQLMNNVPQALQQLAQPTQGTTPSSKLGGLWKTVSPHRSPISNMVSMANNHM.
Mycobacterium tuberculosis H37Rv|Rv3619c|esxV.
Amino acids 2-94.
SEQ ID NO. 4
TINYQFGDVDAHGAMIRAQAGSLEAEHQAIISDVLTASDFWGGAGSAACQGFITQLGRNFQVI
YEQANAHGQKVQAAGNNMAQTDSAVGSSWA.
Mycobacterium tuberculosis H37Rv|Rv3620c|esxW.
Amino acids 2-98.
SEQ ID NO. 5
TSRFMTDPHAMRDMAGRFEVHAQTVEDEARRMWASAQNISGAGWSGMAEATSLDTMTQMNQAF
RNIVNMLHGVRDGLVRDANNYEQQEQASQQILSS.
Mycobacterium tuberculosis H37Rv|Rv3874|esxB|CFP10.
Amino acids 2-100.
SEQ ID NO. 6
AEMKTDAATLAQEAGNFERISGDLKTQIDQVESTAGSLQGQWRGAAGTAAQAAVVRFQEAANK
QKQELDEISTNIRQAGVQYSRADEEQQQALSSQMGF.
Mycobacterium tuberculosis H37Rv|Rv3875|esxA|ESAT-6.
Amino acids 2-95.
SEQ ID NO. 7
TEQQWNFAGIEAAASAIQGNVTSIHSLLDEGKQSLTKLAAAWGGSGSEAYQGVQQKWDATATE
LNNALQNLARTISEAGQAMASTEGNVTGMFA.
Refers to Table 44. The GPGPG spacer (SEQ ID NO. 228)
is underlined. This is one possible combination of Mtb
antigens to create a vaccine cassette. Starting
methionine residue is excluded as it will be included
in the N-terminal signal peptide.
SEQ ID NO. 18
AEMKTDAATLAQEAGNFERISGDLKTQIDQVESTAGSLQGQWRGAAGTAAQAAVVRFQEAANK
QKQELDEISTNIRQAGVQYSRADEEQQQALSSQMGFGPGPGTEQQWNFAGIEAAASAIQGNVT
SIHSLLDEGKQSLTKLAAAWGGSGSEAYQGVQQKWDATATELNNALQNLARTISEAGQAMAST
EGNVTGMFAGPGPGSQIMYNYPAMLGHAGDMAGYAGTLQSLGAEIAVEQAALQSAWQGDTGIT
YQAWQAQWNQAMEDLVRAYHAMSSTHEANTMAMMARDTAEAAKWGGGPGPGPVGGQSSFYSDW
YSPACGKAGCQTYKWETFLTSELPQWLSANRAVKPTGSAAIGLSMAGSSAMILAAYHPQQFIY
AGSLSALLDPSQGMGGPGPGVDFGALPPEINSARMYAGPGSASLVAAAQMWDSVASDLFSAAS
AFQSVVWGLTVGSWIGSSAGLMVAAASPYVAWMSVTAGQAELTAAQVRVAAAAYETAYGLTVP
PPVIAENRAELMILIATNLLGQNTPAIAVNEAEYGEMWAQDAAAMFGYAAATATATATLLPFE
EAPEMTSAGGLLEQAAAVEEASDTAAANQLMNNVPQALQQLAQPTQGTTPSSKLGGLWKTVSP
HRSPISNMVSMANNHMGPGPGTSRFMTDPHAMRDMAGRFEVHAQTVEDEARRMWASAQNISGA
GWSGMAEATSLDTMTQMNQAFRNIVNMLHGVRDGLVRDANNYEQQEQASQQILSSGPGPGTIN
YQFGDVDAHGAMIRAQAGSLEAEHQAIISDVLTASDFWGGAGSAACQGFITQLGRNFQVIYEQ
ANAHGQKVQAAGNNMAQTDSAVGSSWAGPGPGAQIYQAVSAQAAAIHGPGPGPSPSMGRDIKV
QFQSGPGPGGINTIPIAINEAEYVGPGPGAAFQGAHARFVAAAAGPGPGAGWLAFFRDLVARG
LGPGPGASIIRLVGAVLAEQHGPGPGMSFVTTQPEALAAAAGPGPGMHVSFVMAYPEMLAAGP
GPGAYGSFVRTVSLPVGAGPGPGLENDNQLLYNYPGAL.
Refers to Table 45. The GPGPG spacer (SEQ ID NO. 228)
is underlined. This is one possible combination of Mtb
antigens to create a vaccine cassette. Starting
methionine residue is excluded as it will be included
in the N-terminal signal peptide.
SEQ ID NO. 19
AEMKTDAATLAQEAGNFERISGDLKTQIDQVESTAGSLQGQWRGAAGTAAQAAVVRFQEAANK
QKQELDEISTNIRQAGVQYSRADEEQQQALSSQMGFGPGPGTEQQWNFAGIEAAASAIQGNVT
SIHSLLDEGKQSLTKLAAAWGGSGSEAYQGVQQKWDATATELNNALQNLARTISEAGQAMAST
EGNVTGMFAGPGPGTSRFMTDPHAMRDMAGRFEVHAQTVEDEARRMWASAQNISGAGWSGMAE
ATSLDTMTQMNQAFRNIVNMLHGVRDGLVRDANNYEQQEQASQQILSSGPGPGTINYQFGDVD
AHGAMIRAQAGSLEAEHQAIISDVLTASDFWGGAGSAACQGFITQLGRNFQVIYEQANAHGQK
VQAAGNNMAQTDSAVGSSWAGPGPGSQIMYNYPAMLGHAGDMAGYAGTLQSLGAEIAVEQAAL
QSAWQGDTGITYQAWQAQWNQAMEDLVRAYHAMSSTHEANTMAMMARDTAEAAKWGGGPGPGP
VGGQSSFYSDWYSPACGKAGCQTYKWETFLTSELPQWLSANRAVKPTGSAAIGLSMAGSSAMI
LAAYHPQQFIYAGSLSALLDPSQGMGGPGPGVDFGALPPEINSARMYAGPGSASLVAAAQMWD
SVASDLFSAASAFQSVVWGLTVGSWIGSSAGLMVAAASPYVAWMSVTAGQAELTAAQVRVAAA
AYETAYGLTVPPPVIAENRAELMILIATNLLGQNTPAIAVNEAEYGEMWAQDAAAMFGYAAAT
ATATATLLPFEEAPEMTSAGGLLEQAAAVEEASDTAAANQLMNNVPQALQQLAQPTQGTTPSS
KLGGLWKTVSPHRSPISNMVSMANNHMGPGPGAQIYQAVSAQAAAIHGPGPGPSPSMGRDIKV
QFQSGPGPGGINTIPIAINEAEYVGPGPGAAFQGAHARFVAAAAGPGPGAGWLAFFRDLVARG
LGPGPGASIIRLVGAVLAEQHGPGPGMSFVTTQPEALAAAAGPGPGMHVSFVMAYPEMLAAGP
GPGAYGSFVRTVSLPVGAGPGPGLENDNQLLYNYPGAL.
Refers to Table 46. The GPGPG spacer (SEQ ID NO. 228)
is underlined. This is one possible combination of Mtb
antigens to create a vaccine cassette. Starting
methionine residue is excluded as it will be included
in the N-terminal signal peptide.
SEQ ID NO. 20
AEMKTDAATLAQEAGNFERISGDLKTQIDQVESTAGSLQGQWRGAAGTAAQAAVVRFQEAANK
QKQELDEISTNIRQAGVQYSRADEEQQQALSSQMGFGPGPGVDFGALPPEINSARMYAGPGSA
SLVAAAQMWDSVASDLFSAASAFQSVVWGLTVGSWIGSSAGLMVAAASPYVAWMSVTAGQAEL
TAAQVRVAAAAYETAYGLTVPPPVIAENRAELMILIATNLLGQNTPAIAVNEAEYGEMWAQDA
AAMFGYAAATATATATLLPFEEAPEMTSAGGLLEQAAAVEEASDTAAANQLMNNVPQALQQLA
QPTQGTTPSSKLGGLWKTVSPHRSPISNMVSMANNHMGPGPGTEQQWNFAGIEAAASAIQGNV
TSIHSLLDEGKQSLTKLAAAWGGSGSEAYQGVQQKWDATATELNNALQNLARTISEAGQAMAS
TEGNVTGMFAGPGPGTSRFMTDPHAMRDMAGRFEVHAQTVEDEARRMWASAQNISGAGWSGMA
EATSLDTMTQMNQAFRNIVNMLHGVRDGLVRDANNYEQQEQASQQILSSGPGPGSQIMYNYPA
MLGHAGDMAGYAGTLQSLGAEIAVEQAALQSAWQGDTGITYQAWQAQWNQAMEDLVRAYHAMS
STHEANTMAMMARDTAEAAKWGGGPGPGTINYQFGDVDAHGAMIRAQAGSLEAEHQAIISDVL
TASDFWGGAGSAACQGFITQLGRNFQVIYEQANAHGQKVQAAGNNMAQTDSAVGSSWAGPGPG
PVGGQSSFYSDWYSPACGKAGCQTYKWETFLTSELPQWLSANRAVKPTGSAAIGLSMAGSSAM
ILAAYHPQQFIYAGSLSALLDPSQGMGGPGPGAQIYQAVSAQAAAIHGPGPGPSPSMGRDIKV
QFQSGPGPGGINTIPIAINEAEYVGPGPGAAFQGAHARFVAAAAGPGPGAGWLAFFRDLVARG
LGPGPGASIIRLVGAVLAEQHGPGPGMSFVTTQPEALAAAAGPGPGMHVSFVMAYPEMLAAGP
GPGAYGSFVRTVSLPVGAGPGPGLENDNQLLYNYPGAL.
The N-terminal signal peptide of human LAMP-1. Uniprot
ID P11279. Amino acids 1-28.
SEQ ID NO. 21
MAAPGSARRPLLLLLLLLLLGLMHCASA.
The C-terminal transmembrane and cytoplasmic domains of
human LAMP-1. Uniprot ID P11279. Amino acids 383-417.
SEQ ID NO. 22
LIPIAVGGALAGLVLIVLIAYLVGRKRSHAGYQTI.
An N-terminal signal peptide (sec domain) of human HLA-B.
Uniprot P01889|HLAB HUMAN HLA class I histocompatibility
antigen, B alpha chain. Amino acids 1-24
SEQ ID NO. 23
MLVMAPRTVLLLLSAALALTETWA.
An N-terminal signal peptide (sec domain) of human HLA-B.
Amino acids 1-26. Identified from patent US20180177885A1.
Combination of amino acid sequences from multiple NCBI-
deposited sequences: accession numbers UOA00096.1 and
BBD34030.1.
SEQ ID NO. 24
MRVTAPRTLILLLSGALALTETWAGS.
A C-terminal transmembrane/cytoplasmic domain of human
HLA-B. Also called the MHC class I trafficking domain
(MITD). Uniprot P01889|HLAB_HUMAN HLA class I histo-
compatibility antigen, B alpha chain. Amino acids 310-362
SEQ ID NO. 25
GIVAGLAVLAVVVIGAVVAAVMCRRKSSGGKGGSYSQAACSDSAQGSDVSLTA.
A C-terminal transmembrane/cytoplasmic domain of human
HLA class I. Also called the MHC class I trafficking
domain (MITD). NCBI GenBank accession ADA70350.2.
SEQ ID NO. 26
IVGIVAGLAVLAVVVIGAVVATVMCRRKSSGGKGGSYSQAASSDSAQGSDVSLTA.
An N-terminal signal peptide from the MHC class II HLA-
DRB1 protein. Uniprot P01911|DRB1_HUMAN HLA class II
histocompatibility antigen, DRB1 beta chain. Amino acids
1-29.
SEQ ID NO. 27
MVCLKLPGGSCMTALTVTLMVLSSPLALS.
A C-terminal transmembrane/cytoplasmic domain of human
MHC class II HLA-DRB1 protein. Uniprot P01911|DRB1_HUMAN
HLA class II histocompatibility antigen, DRB1 beta
chain. Amino acids 228-266.
SEQ ID NO. 28
MLSGVGGFVLGLLFLGAGLFIYFRNQKGHSGLQPTGFLS.
An N-terminal signal peptide from the MHC class II HLA-
DRαprotein. Uniprot P01903. Amino acids 1-25
SEQ ID 29
MAISGVPVLGFFIIAVLMSAQESWA.
The tissue-type plasminogen activator (tPA) signal
peptide with a P22A mutation. See Uniprot entry P00750
for the native human tPA signal peptide sequence with
an alanine substituted for proline at position 22.
SEQ ID NO. 30
MDAMKRGLCCVLLLCGAVFVSA
An optimized Kozak sequence.
GCCACC.
Human hemoglobin subunit beta (HBB) untranslated
regions (UTRs). NCBI reference nucleotide sequence
NM_000518.5. The 5′ UTR is underlined and spans
from nucleotides 1-44. The 3′ UTR is underlined
and spans from nucleotides 495-628.
SEQ ID NO. 32
ACAUUUGCUUCUGACACAACUGUGUUCACUAGCAACCUCAAACAGACACCAUGGUGCAUCUGA
CUCCUGAGGAGAAGUCUGCCGUUACUGCCCUGUGGGGCAAGGUGAACGUGGAUGAAGUUGGUG
GUGAGGCCCUGGGCAGGCUGCUGGUGGUCUACCCUUGGACCCAGAGGUUCUUUGAGUCCUUUG
GGGAUCUGUCCACUCCUGAUGCUGUUAUGGGCAACCCUAAGGUGAAGGCUCAUGGCAAGAAAG
UGCUCGGUGCCUUUAGUGAUGGCCUGGCUCACCUGGACAACCUCAAGGGCACCUUUGCCACAC
UGAGUGAGCUGCACUGUGACAAGCUGCACGUGGAUCCUGAGAACUUCAGGCUCCUGGGCAACG
UGCUGGUCUGUGUGCUGGCCCAUCACUUUGGCAAAGAAUUCACCCCACCAGUGCAGGCUGCCU
AUCAGAAAGUGGUGGCUGGUGUGGCUAAUGCCCUGGCCCACAAGUAUCACUAAGCUCGCUUUC
UUGCUGUCCAAUUUCUAUUAAAGGUUCCUUUGUUCCCUAAGUCCAACUACUAAACUGGGGGAU
AUUAUGAAGGGCCUUGAGCAUCUGGAUUCUGCCUAAUAAAAAACAUUUAUUUUCAUUGCAA.
An Mtb CD4 T cell-focused vaccine construct. Protein
sequence consisting of SEQ ID NO. 18 (associated with
Table 44) and the LAMP-1 signal sequences (underlined;
SEQ ID NOs. 21 and 22).
SEQ ID NO. 33
MAAPGSARRPLLLLLLLLLLGLMHCASAAEMKTDAATLAQEAGNFERISGDLKTQIDQVESTA
GSLQGQWRGAAGTAAQAAVVRFQEAANKQKQELDEISTNIRQAGVQYSRADEEQQQALSSQMG
FGPGPGTEQQWNFAGIEAAASAIQGNVTSIHSLLDEGKQSLTKLAAAWGGSGSEAYQGVQQKW
DATATELNNALQNLARTISEAGQAMASTEGNVTGMFAGPGPGSQIMYNYPAMLGHAGDMAGYA
GTLQSLGAEIAVEQAALQSAWQGDTGITYQAWQAQWNQAMEDLVRAYHAMSSTHEANTMAMMA
RDTAEAAKWGGGPGPGPVGGQSSFYSDWYSPACGKAGCQTYKWETFLTSELPQWLSANRAVKP
TGSAAIGLSMAGSSAMILAAYHPQQFIYAGSLSALLDPSQGMGGPGPGVDFGALPPEINSARM
YAGPGSASLVAAAQMWDSVASDLFSAASAFQSVVWGLTVGSWIGSSAGLMVAAASPYVAWMSV
TAGQAELTAAQVRVAAAAYETAYGLTVPPPVIAENRAELMILIATNLLGQNTPAIAVNEAEYG
EMWAQDAAAMFGYAAATATATATLLPFEEAPEMTSAGGLLEQAAAVEEASDTAAANQLMNNVP
QALQQLAQPTQGTTPSSKLGGLWKTVSPHRSPISNMVSMANNHMGPGPGTSRFMTDPHAMRDM
AGRFEVHAQTVEDEARRMWASAQNISGAGWSGMAEATSLDTMTQMNQAFRNIVNMLHGVRDGL
VRDANNYEQQEQASQQILSSGPGPGTINYQFGDVDAHGAMIRAQAGSLEAEHQAIISDVLTAS
DFWGGAGSAACQGFITQLGRNFQVIYEQANAHGQKVQAAGNNMAQTDSAVGSSWAGPGPGAQI
YQAVSAQAAAIHGPGPGPSPSMGRDIKVQFQSGPGPGGINTIPIAINEAEYVGPGPGAAFQGA
HARFVAAAAGPGPGAGWLAFFRDLVARGLGPGPGASIIRLVGAVLAEQHGPGPGMSFVTTQPE
ALAAAAGPGPGMHVSFVMAYPEMLAAGPGPGAYGSFVRTVSLPVGAGPGPGLENDNQLLYNYP
GALLIPIAVGGALAGLVLIVLIAYLVGRKRSHAGYQTI.
Codon-optimized forward nucleotide sequence
corresponding to the SEQ ID NO. 33 protein sequence.
The HBB UTRs are underlined. N1-methylpseudouridine
is substituted for all uridines (U) during mRNA
synthesis.
SEQ ID NO. 34
ACAUUUGCUUCUGACACAACUGUGUUCACUAGCAACCUCAAACAGCCACCAUGGCCGCUCCUG
GCUCCGCGCGCAGACCGUUGCUCCUUCUCUUACUACUGCUGCUGUUGGGACUUAUGCACUGCG
CUUCCGCCGCUGAGAUGAAAACCGACGCCGCGACCCUGGCCCAGGAAGCCGGAAACUUCGAGC
GGAUUUCCGGAGAUCUUAAGACUCAGAUCGACCAGGUCGAAUCCACCGCCGGAUCGCUCCAGG
GACAGUGGAGAGGCGCCGCGGGAACCGCGGCCCAGGCCGCCGUCGUGCGGUUCCAAGAGGCCG
CCAACAAGCAGAAGCAGGAACUGGACGAAAUUUCCACUAACAUUCGCCAAGCUGGCGUGCAGU
ACUCGAGAGCCGAUGAAGAACAGCAGCAAGCCCUCUCCUCACAAAUGGGUUUCGGACCGGGAC
CCGGCACCGAACAACAGUGGAACUUCGCCGGUAUUGAGGCCGCCGCCUCGGCGAUUCAGGGAA
AUGUCACUUCCAUUCAUUCCCUGCUCGACGAGGGAAAACAGAGCCUCACCAAGCUUGCAGCUG
CCUGGGGUGGUUCGGGCUCCGAAGCCUACCAGGGGGUGCAGCAGAAGUGGGAUGCGACUGCCA
CCGAACUGAACAACGCGCUCCAAAAUCUGGCUCGGACCAUUAGCGAAGCUGGUCAGGCUAUGG
CGUCCACCGAGGGCAACGUGACCGGAAUGUUCGCAGGCCCGGGUCCCGGAUCCCAAAUCAUGU
AUAACUACCCAGCAAUGCUGGGCCAUGCCGGCGACAUGGCGGGGUACGCCGGCACCUUACAGU
CUCUAGGCGCUGAAAUCGCCGUCGAACAAGCCGCCCUGCAAUCCGCCUGGCAAGGAGAUACCG
GAAUUACCUAUCAGGCUUGGCAGGCGCAAUGGAACCAGGCCAUGGAGGACCUCGUGCGGGCCU
ACCACGCCAUGUCAAGCACCCACGAAGCCAACACUAUGGCCAUGAUGGCUAGAGAUACUGCAG
AGGCUGCCAAGUGGGGAGGCGGCCCGGGACCCGGACCUGUGGGAGGCCAGUCCAGCUUCUACA
GCGACUGGUACAGCCCAGCCUGCGGAAAGGCCGGCUGCCAAACAUACAAGUGGGAAACCUUCC
UUACCUCCGAACUCCCCCAGUGGCUUUCCGCGAACCGGGCCGUGAAGCCGACUGGAUCAGCAG
CCAUCGGCCUUAGCAUGGCGGGCUCGUCGGCCAUGAUUCUGGCCGCAUACCAUCCUCAGCAAU
UCAUCUACGCCGGAUCGCUUUCCGCCCUGCUGGACCCCUCACAGGGUAUGGGUGGACCUGGGC
CCGGCGUGGACUUCGGAGCCCUGCCACCUGAAAUCAACUCCGCCCGGAUGUACGCAGGACCUG
GAUCCGCCAGCCUGGUGGCGGCCGCGCAGAUGUGGGAUAGCGUGGCCUCCGACCUGUUCAGCG
CGGCUUCAGCUUUCCAAAGCGUGGUCUGGGGACUGACUGUGGGAUCCUGGAUCGGAUCAAGCG
CAGGCCUCAUGGUGGCCGCGGCGUCCCCUUACGUGGCCUGGAUGAGCGUGACCGCCGGCCAGG
CAGAACUGACCGCAGCCCAAGUCCGCGUGGCCGCAGCCGCCUACGAGACUGCCUACGGUCUGA
CGGUGCCGCCGCCAGUGAUCGCCGAGAACAGAGCAGAGCUCAUGAUCCUCAUCGCGACCAACC
UACUGGGCCAGAACACUCCGGCGAUUGCCGUGAACGAAGCCGAAUAUGGGGAGAUGUGGGCUC
AGGACGCUGCAGCCAUGUUCGGAUAUGCGGCCGCGACUGCUACCGCCACCGCCACUCUGCUGC
CGUUCGAGGAGGCCCCCGAAAUGACUUCCGCGGGAGGCCUGUUGGAGCAAGCUGCCGCGGUGG
AGGAAGCUAGCGACACCGCCGCAGCCAACCAGCUCAUGAACAACGUGCCACAGGCCCUGCAGC
AGCUGGCCCAGCCCACUCAAGGGACCACCCCGAGCUCAAAGCUGGGCGGUCUGUGGAAAACCG
UGUCCCCCCACCGCUCGCCCAUUUCCAACAUGGUGUCAAUGGCGAACAACCACAUGGGCCCUG
GGCCGGGCACCUCGCGGUUCAUGACCGAUCCUCAUGCUAUGCGGGAUAUGGCUGGACGGUUCG
AGGUGCACGCCCAAACUGUGGAGGACGAGGCCCGCCGGAUGUGGGCCAGCGCGCAGAACAUCU
CGGGGGCCGGCUGGUCCGGAAUGGCCGAAGCCACCUCCCUCGAUACCAUGACCCAGAUGAACC
AGGCCUUCCGGAACAUUGUGAACAUGCUGCAUGGAGUGCGGGACGGCCUGGUCCGGGACGCCA
ACAAUUACGAGCAGCAGGAGCAGGCCUCUCAGCAAAUCCUGUCCAGCGGACCGGGGCCGGGAA
CCAUAAACUACCAGUUUGGCGACGUCGACGCCCACGGAGCCAUGAUCAGGGCGCAGGCCGGGU
CGCUGGAAGCAGAACACCAGGCCAUCAUCUCCGAUGUGCUGACCGCCUCCGACUUUUGGGGAG
GAGCCGGUUCGGCUGCCUGCCAAGGGUUCAUCACACAAUUGGGAAGGAACUUCCAGGUCAUCU
ACGAGCAGGCCAAUGCACACGGUCAAAAGGUCCAAGCGGCGGGCAACAACAUGGCCCAGACUG
ACUCGGCCGUGGGCAGCAGUUGGGCCGGACCGGGCCCUGGGGCCCAGAUCUACCAAGCAGUGU
CGGCCCAAGCCGCAGCGAUCCAUGGACCAGGACCGGGACCGUCCCCGAGCAUGGGCCGGGAUA
UUAAGGUUCAGUUCCAGUCCGGACCCGGCCCGGGGGGUAUUAACACCAUUCCGAUUGCCAUUA
AUGAGGCAGAAUACGUCGGCCCUGGUCCCGGCGCCGCGUUCCAAGGAGCCCACGCACGAUUCG
UGGCGGCAGCUGCGGGACCCGGGCCUGGUGCCGGUUGGCUGGCUUUUUUCCGCGACCUGGUGG
CCAGAGGACUUGGACCCGGUCCGGGCGCCAGCAUUAUCAGGCUGGUCGGGGCCGUGCUGGCGG
AACAGCAUGGACCUGGCCCGGGAAUGAGCUUCGUGACCACCCAACCUGAGGCGCUUGCCGCGG
CGGCAGGCCCUGGACCCGGGAUGCAUGUGUCUUUUGUGAUGGCCUACCCGGAAAUGCUGGCCG
CGGGUCCGGGCCCCGGAGCCUACGGAUCAUUCGUGCGCACUGUGUCCCUCCCCGUGGGAGCCG
GACCUGGACCGGGGUUGGAGAAUGAUAACCAGCUGCUCUACAACUAUCCGGGGGCCCUCCUGA
UCCCCAUCGCCGUGGGCGGGGCCCUGGCCGGGCUGGUUCUGAUCGUCCUGAUUGCAUACCUGG
UCGGACGGAAGCGCUCUCACGCCGGCUACCAAACCAUCUGAUGAUAAUAGGCUCGCUUUCUUG
CUGUCCAAUUUCUAUUAAAGGUUCCUUUGUUCCCUAAGUCCAACUACUAAACUGGGGGAUAUU
AUGAAGGGCCUUGAGCAUCUGGAUUCUGCCUAAUAAAAAACAUUUAUUUUCAUUGCAA.
An Mtb CD4 T cell-focused vaccine construct. Protein
sequence consisting of SEQ ID 19 (associated with Table
45) and the LAMP-1 signal sequences (underlined; SEQ ID
NOs. 21 and 22).
SEQ ID NO. 35
MAAPGSARRPLLLLLLLLLLGLMHCASAAEMKTDAATLAQEAGNFERISGDLKTQIDQVESTA
GSLQGQWRGAAGTAAQAAVVRFQEAANKQKQELDEISTNIRQAGVQYSRADEEQQQALSSQMG
FGPGPGTEQQWNFAGIEAAASAIQGNVTSIHSLLDEGKQSLTKLAAAWGGSGSEAYQGVQQKW
DATATELNNALQNLARTISEAGQAMASTEGNVTGMFAGPGPGTSRFMTDPHAMRDMAGRFEVH
AQTVEDEARRMWASAQNISGAGWSGMAEATSLDTMTQMNQAFRNIVNMLHGVRDGLVRDANNY
EQQEQASQQILSSGPGPGTINYQFGDVDAHGAMIRAQAGSLEAEHQAIISDVLTASDFWGGAG
SAACQGFITQLGRNFQVIYEQANAHGQKVQAAGNNMAQTDSAVGSSWAGPGPGSQIMYNYPAM
LGHAGDMAGYAGTLQSLGAEIAVEQAALQSAWQGDTGITYQAWQAQWNQAMEDLVRAYHAMSS
THEANTMAMMARDTAEAAKWGGGPGPGPVGGQSSFYSDWYSPACGKAGCQTYKWETFLTSELP
QWLSANRAVKPTGSAAIGLSMAGSSAMILAAYHPQQFIYAGSLSALLDPSQGMGGPGPGVDFG
ALPPEINSARMYAGPGSASLVAAAQMWDSVASDLFSAASAFQSVVWGLTVGSWIGSSAGLMVA
AASPYVAWMSVTAGQAELTAAQVRVAAAAYETAYGLTVPPPVIAENRAELMILIATNLLGQNT
PAIAVNEAEYGEMWAQDAAAMFGYAAATATATATLLPFEEAPEMTSAGGLLEQAAAVEEASDT
AAANQLMNNVPQALQQLAQPTQGTTPSSKLGGLWKTVSPHRSPISNMVSMANNHMGPGPGAQI
YQAVSAQAAAIHGPGPGPSPSMGRDIKVQFQSGPGPGGINTIPIAINEAEYVGPGPGAAFQGA
HARFVAAAAGPGPGAGWLAFFRDLVARGLGPGPGASIIRLVGAVLAEQHGPGPGMSFVTTQPE
ALAAAAGPGPGMHVSFVMAYPEMLAAGPGPGAYGSFVRTVSLPVGAGPGPGLENDNQLLYNYP
GALLIPIAVGGALAGLVLIVLIAYLVGRKRSHAGYQTI.
Codon-optimized forward nucleotide sequence
corresponding to the SEQ ID NO. 35 protein
sequence. The HBB UTRs are underlined. N1-
methylpseudouridine is substituted for all
uridines (U) during mRNA synthesis.
SEQ ID NO. 36
ACAUUUGCUUCUGACACAACUGUGUUCACUAGCAACCUCAAACAGCCACCAUGGCCGCUCCAG
GCUCAGCCCGCAGACCCUUGCUCCUCCUGUUACUACUGCUGCUGCUCGGGUUGAUGCACUGCG
CGUCCGCCGCUGAGAUGAAAACCGACGCCGCGACCCUGGCCCAGGAAGCCGGAAACUUCGAGC
GGAUUUCCGGAGAUCUUAAGACUCAGAUCGACCAGGUCGAAUCCACCGCCGGAUCGCUCCAGG
GACAGUGGAGAGGCGCCGCGGGAACCGCGGCCCAGGCCGCCGUCGUGCGGUUCCAAGAGGCCG
CCAACAAGCAGAAGCAGGAACUGGACGAAAUUUCCACUAACAUUCGCCAAGCUGGCGUGCAGU
ACUCGAGAGCCGAUGAAGAACAGCAGCAAGCCCUCUCCUCACAAAUGGGUUUCGGUCCUGGAC
CUGGAACGGAGCAGCAGUGGAACUUCGCCGGGAUCGAAGCCGCCGCCUCGGCUAUCCAAGGGA
AUGUCACCUCGAUCCAUUCCCUUCUGGACGAAGGAAAGCAGUCCCUGACCAAGCUUGCGGCAG
CCUGGGGCGGAAGCGGCAGCGAAGCCUACCAGGGCGUGCAGCAAAAAUGGGACGCAACCGCCA
CUGAGCUGAACAACGCCCUCCAAAACCUGGCUAGAACUAUUUCCGAGGCCGGACAGGCUAUGG
CCAGCACCGAGGGCAACGUGACCGGGAUGUUCGCUGGCCCUGGGCCGGGCACCUCGCGGUUCA
UGACCGAUCCUCAUGCUAUGCGGGAUAUGGCUGGACGGUUCGAGGUGCACGCCCAAACUGUGG
AGGACGAGGCCCGCCGGAUGUGGGCCAGCGCGCAGAACAUCUCGGGGGCCGGCUGGUCCGGAA
UGGCCGAAGCCACCUCCCUCGAUACCAUGACCCAGAUGAACCAGGCCUUCCGGAACAUUGUGA
ACAUGCUGCAUGGAGUGCGGGACGGCCUGGUCCGGGACGCCAACAAUUACGAGCAGCAGGAGC
AGGCCUCUCAGCAAAUCCUGUCCAGCGGACCUGGCCCCGGUACCAUCAACUACCAGUUCGGCG
ACGUGGACGCACACGGCGCCAUGAUCAGGGCCCAGGCUGGGUCGCUAGAAGCCGAGCACCAGG
CUAUCAUUUCUGAUGUCCUCACCGCGUCAGAUUUUUGGGGGGGGGGGGGUCAGCCGCCUGCCA
GGGAUUCAUAACCCAGCUCGGCCGGAACUUCCAGGUCAUCUACGAACAGGCCAACGCUCACGG
UCAGAAGGUGCAGGCGGCCGGUAACAACAUGGCCCAGACUGACUCCGCCGUGGGGUCAAGCUG
GGCCGGGCCGGGGCCUGGAUCACAAAUCAUGUACAACUACCCGGCAAUGCUGGGCCACGCUGG
AGACAUGGCCGGCUACGCCGGCACGCUGCAGUCACUCGGUGCCGAGAUCGCCGUCGAGCAGGC
GGCACUGCAGAGCGCUUGGCAGGGAGACACUGGCAUUACCUACCAAGCGUGGCAGGCUCAGUG
GAAUCAAGCUAUGGAAGAUCUGGUCCGCGCGUACCACGCCAUGUCCUCCACUCACGAAGCCAA
CACCAUGGCUAUGAUGGCCAGGGACACUGCCGAGGCAGCCAAAUGGGGGGGAGGUCCCGGCCC
UGGCCCUGUCGGGGGGCAGUCAUCCUUCUACUCCGACUGGUACUCCCCCGCCUGCGGAAAGGC
CGGCUGUCAGACCUAUAAGUGGGAAACCUUCCUGACCUCCGAACUCCCGCAGUGGCUCUCGGC
GAAUCGGGCAGUGAAGCCCACCGGGUCGGCGGCUAUUGGACUGUCCAUGGCCGGUUCCAGCGC
GAUGAUCCUGGCCGCCUAUCACCCCCAGCAAUUCAUCUACGCUGGCUCCCUGUCCGCCCUGCU
UGACCCAUCGCAGGGGAUGGGUGGCCCGGGACCGGGAGUGGACUUCGGGGCCUUGCCACCCGA
AAUCAACUCUGCCAGAAUGUACGCCGGGCCUGGAUCCGCGUCCCUUGUAGCCGCGGCCCAGAU
GUGGGACUCCGUGGCCUCAGACCUGUUCUCCGCCGCGUCGGCGUUUCAGUCCGUGGUGUGGGG
GCUGACUGUGGGCAGCUGGAUCGGAAGCUCCGCCGGACUGAUGGUCGCCGCCGCCAGCCCGUA
CGUGGCUUGGAUGAGCGUCACUGCCGGGCAGGCCGAGCUCACUGCCGCACAGGUCCGCGUCGC
GGCCGCAGCCUACGAAACCGCAUACGGACUGACAGUGCCCCCCCCGGUGAUCGCCGAGAACCG
GGCGGAACUGAUGAUCCUUAUUGCCACCAACCUCUUGGGACAAAAUACCCCCGCCAUUGCGGU
GAACGAGGCGGAGUAUGGAGAGAUGUGGGCCCAGGACGCCGCGGCCAUGUUUGGGUACGCCGC
CGCAACCGCGACUGCCACCGCCACGCUGCUGCCGUUCGAGGAGGCCCCCGAAAUGACCUCGGC
AGGAGGGCUGCUCGAGCAGGCCGCCGCUGUGGAAGAGGCAUCCGAUACUGCCGCUGCCAACCA
ACUCAUGAACAAUGUGCCUCAGGCCCUGCAGCAGCUGGCCCAGCCCACCCAGGGAACCACCCC
GAGCUCGAAGCUUGGAGGGCUGUGGAAAACGGUGUCACCGCACCGGUCCCCCAUUUCCAACAU
GGUGUCGAUGGCCAACAACCAUAUGGGACCGGGCCCUGGGGCCCAGAUCUACCAAGCAGUGUC
GGCCCAAGCCGCAGCGAUCCAUGGACCAGGACCGGGACCGUCCCCGAGCAUGGGCCGGGAUAU
UAAGGUUCAGUUCCAGUCCGGACCCGGCCCGGGGGGUAUUAACACCAUUCCGAUUGCCAUUAA
UGAGGCAGAAUACGUCGGCCCUGGUCCCGGCGCCGCGUUCCAAGGAGCCCACGCACGAUUCGU
GGCGGCAGCUGCGGGACCCGGGCCUGGUGCCGGUUGGCUGGCUUUUUUCCGCGACCUGGUGGC
CAGAGGACUUGGACCCGGUCCGGGCGCCAGCAUUAUCAGGCUGGUCGGGGCCGUGCUGGCGGA
ACAGCAUGGACCUGGCCCGGGAAUGAGCUUCGUGACCACCCAACCUGAGGCGCUUGCCGCGGC
GGCAGGCCCUGGACCCGGGAUGCAUGUGUCUUUUGUGAUGGCCUACCCGGAAAUGCUGGCCGC
GGGUCCGGGCCCCGGAGCCUACGGAUCAUUCGUGCGCACUGUGUCCCUCCCCGUGGGAGCCGG
ACCUGGACCGGGGUUGGAGAAUGAUAACCAGCUGCUCUACAACUAUCCGGGGGCCCUCCUCAU
CCCAAUCGCCGUGGGCGGAGCCCUGGCCGGACUGGUGCUCAUUGUCCUGAUCGCCUACCUUGU
GGGGAGAAAACGGUCCCAUGCGGGAUAUCAAACGAUCUGAUGAUAAUAGGCUCGCUUUCUUGC
UGUCCAAUUUCUAUUAAAGGUUCCUUUGUUCCCUAAGUCCAACUACUAAACUGGGGGAUAUUA
UGAAGGGCCUUGAGCAUCUGGAUUCUGCCUAAUAAAAAACAUUUAUUUUCAUUGCAA.
An Mtb CD4 T cell-focused vaccine construct. Protein
sequence consisting of SEQ ID NO. 20 (associated with
Table 46) and the HLA class I sec/MITD signal sequences
(underlined; SEQ ID NOs. 24 and 25).
SEQ ID NO. 37
MRVTAPRTLILLLSGALALTETWAAEMKTDAATLAQEAGNFERISGDLKTQIDQVESTAGSLQ
GQWRGAAGTAAQAAVVRFQEAANKQKQELDEISTNIRQAGVQYSRADEEQQQALSSQMGFGPG
PGVDFGALPPEINSARMYAGPGSASLVAAAQMWDSVASDLFSAASAFQSVVWGLTVGSWIGSS
AGLMVAAASPYVAWMSVTAGQAELTAAQVRVAAAAYETAYGLTVPPPVIAENRAELMILIATN
LLGQNTPAIAVNEAEYGEMWAQDAAAMFGYAAATATATATLLPFEEAPEMTSAGGLLEQAAAV
EEASDTAAANQLMNNVPQALQQLAQPTQGTTPSSKLGGLWKTVSPHRSPISNMVSMANNHMGP
GPGTEQQWNFAGIEAAASAIQGNVTSIHSLLDEGKQSLTKLAAAWGGSGSEAYQGVQQKWDAT
ATELNNALQNLARTISEAGQAMASTEGNVTGMFAGPGPGTSRFMTDPHAMRDMAGRFEVHAQT
VEDEARRMWASAQNISGAGWSGMAEATSLDTMTQMNQAFRNIVNMLHGVRDGLVRDANNYEQQ
EQASQQILSSGPGPGSQIMYNYPAMLGHAGDMAGYAGTLQSLGAEIAVEQAALQSAWQGDTGI
TYQAWQAQWNQAMEDLVRAYHAMSSTHEANTMAMMARDTAEAAKWGGGPGPGTINYQFGDVDA
HGAMIRAQAGSLEAEHQAIISDVLTASDFWGGAGSAACQGFITQLGRNFQVIYEQANAHGQKV
QAAGNNMAQTDSAVGSSWAGPGPGPVGGQSSFYSDWYSPACGKAGCQTYKWETFLTSELPQWL
SANRAVKPTGSAAIGLSMAGSSAMILAAYHPQQFIYAGSLSALLDPSQGMGGPGPGAQIYQAV
SAQAAAIHGPGPGPSPSMGRDIKVQFQSGPGPGGINTIPIAINEAEYVGPGPGAAFQGAHARF
VAAAAGPGPGAGWLAFFRDLVARGLGPGPGASIIRLVGAVLAEQHGPGPGMSFVTTQPEALAA
AAGPGPGMHVSFVMAYPEMLAAGPGPGAYGSFVRTVSLPVGAGPGPGLENDNQLLYNYPGALG
IVAGLAVLAVVVIGAVVAAVMCRRKSSGGKGGSYSQAACSDSAQGSDVSLTA.
Codon optimized forward nucleotide sequence
corresponding to the SEQ ID NO. 37 protein
sequence. The HBB UTRs are underlined. N1-
methylpseudouridine is substituted for all
uridines (U) during mRNA synthesis.
SEQ ID NO. 38
ACAUUUGCUUCUGACACAACUGUGUUCACUAGCAACCUCAAACAGCCACCAUGCGGGUCACUG
CCCCCAGAACCUUGAUUUUACUACUGUCGGGAGCCCUCGCUCUGACUGAAACAUGGGCCGCUG
AGAUGAAAACCGACGCCGCGACCCUGGCCCAGGAAGCCGGAAACUUCGAGCGGAUUUCCGGAG
AUCUUAAGACUCAGAUCGACCAGGUCGAAUCCACCGCCGGAUCGCUCCAGGGACAGUGGAGAG
GCGCCGCGGGAACCGCGGCCCAGGCCGCCGUCGUGCGGUUCCAAGAGGCCGCCAACAAGCAGA
AGCAGGAACUGGACGAAAUUUCCACUAACAUUCGCCAAGCUGGCGUGCAGUACUCGAGAGCCG
AUGAAGAACAGCAGCAAGCCCUCUCCUCACAAAUGGGUUUCGGACCUGGGCCCGGCGUGGACU
UCGGAGCCCUGCCACCUGAAAUCAACUCCGCCCGGAUGUACGCAGGACCUGGAUCCGCCAGCC
UGGUGGCGGCCGCGCAGAUGUGGGAUAGCGUGGCCUCCGACCUGUUCAGCGCGGCUUCAGCUU
UCCAAAGCGUGGUCUGGGGACUGACUGUGGGAUCCUGGAUCGGAUCAAGCGCAGGCCUCAUGG
UGGCCGCGGCGUCCCCUUACGUGGCCUGGAUGAGCGUGACCGCCGGCCAGGCAGAACUGACCG
CAGCCCAAGUCCGCGUGGCCGCAGCCGCCUACGAGACUGCCUACGGUCUGACGGUGCCGCCGC
CAGUGAUCGCCGAGAACAGAGCAGAGCUCAUGAUCCUCAUCGCGACCAACCUACUGGGCCAGA
ACACUCCGGCGAUUGCCGUGAACGAAGCCGAAUAUGGGGAGAUGUGGGCUCAGGACGCUGCAG
CCAUGUUCGGAUAUGCGGCCGCGACUGCUACCGCCACCGCCACUCUGCUGCCGUUCGAGGAGG
CCCCCGAAAUGACUUCCGCGGGAGGCCUGUUGGAGCAAGCUGCCGCGGUGGAGGAAGCUAGCG
ACACCGCCGCAGCCAACCAGCUCAUGAACAACGUGCCACAGGCCCUGCAGCAGCUGGCCCAGC
CCACUCAAGGGACCACCCCGAGCUCAAAGCUGGGCGGUCUGUGGAAAACCGUGUCCCCCCACC
GCUCGCCCAUUUCCAACAUGGUGUCAAUGGCGAACAACCACAUGGGUCCUGGACCUGGAACGG
AGCAGCAGUGGAACUUCGCCGGGAUCGAAGCCGCCGCCUCGGCUAUCCAAGGGAAUGUCACCU
CGAUCCAUUCCCUUCUGGACGAAGGAAAGCAGUCCCUGACCAAGCUUGCGGCAGCCUGGGGCG
GAAGCGGCAGCGAAGCCUACCAGGGCGUGCAGCAAAAAUGGGACGCAACCGCCACUGAGCUGA
ACAACGCCCUCCAAAACCUGGCUAGAACUAUUUCCGAGGCCGGACAGGCUAUGGCCAGCACCG
AGGGCAACGUGACCGGGAUGUUCGCUGGCCCUGGGCCGGGCACCUCGCGGUUCAUGACCGAUC
CUCAUGCUAUGCGGGAUAUGGCUGGACGGUUCGAGGUGCACGCCCAAACUGUGGAGGACGAGG
CCCGCCGGAUGUGGGCCAGCGCGCAGAACAUCUCGGGGGCCGGCUGGUCCGGAAUGGCCGAAG
CCACCUCCCUCGAUACCAUGACCCAGAUGAACCAGGCCUUCCGGAACAUUGUGAACAUGCUGC
AUGGAGUGCGGGACGGCCUGGUCCGGGACGCCAACAAUUACGAGCAGCAGGAGCAGGCCUCUC
AGCAAAUCCUGUCCAGCGGGCCGGGGCCUGGAUCACAAAUCAUGUACAACUACCCGGCAAUGC
UGGGCCACGCUGGAGACAUGGCCGGCUACGCCGGCACGCUGCAGUCACUCGGUGCCGAGAUCG
CCGUCGAGCAGGCGGCACUGCAGAGCGCUUGGCAGGGAGACACUGGCAUUACCUACCAAGCGU
GGCAGGCUCAGUGGAAUCAAGCUAUGGAAGAUCUGGUCCGCGCGUACCACGCCAUGUCCUCCA
CUCACGAAGCCAACACCAUGGCUAUGAUGGCCAGGGACACUGCCGAGGCAGCCAAAUGGGGGG
GAGGACCGGGGCCGGGAACCAUAAACUACCAGUUUGGCGACGUCGACGCCCACGGAGCCAUGA
UCAGGGCGCAGGCCGGGUCGCUGGAAGCAGAACACCAGGCCAUCAUCUCCGAUGUGCUGACCG
CCUCCGACUUUUGGGGAGGAGCCGGUUCGGCUGCCUGCCAAGGGUUCAUCACACAAUUGGGAA
GGAACUUCCAGGUCAUCUACGAGCAGGCCAAUGCACACGGUCAAAAGGUCCAAGCGGCGGGCA
ACAACAUGGCCCAGACUGACUCGGCCGUGGGCAGCAGUUGGGCCGGUCCCGGCCCUGGCCCUG
UCGGGGGGCAGUCAUCCUUCUACUCCGACUGGUACUCCCCCGCCUGCGGAAAGGCCGGCUGUC
AGACCUAUAAGUGGGAAACCUUCCUGACCUCCGAACUCCCGCAGUGGCUCUCGGCGAAUCGGG
CAGUGAAGCCCACCGGGUCGGCGGCUAUUGGACUGUCCAUGGCCGGUUCCAGCGCGAUGAUCC
UGGCCGCCUAUCACCCCCAGCAAUUCAUCUACGCUGGCUCCCUGUCCGCCCUGCUUGACCCAU
CGCAGGGGAUGGGUGGACCGGGCCCUGGGGCCCAGAUCUACCAAGCAGUGUCGGCCCAAGCCG
CAGCGAUCCAUGGACCAGGACCGGGACCGUCCCCGAGCAUGGGCCGGGAUAUUAAGGUUCAGU
UCCAGUCCGGACCCGGCCCGGGGGGUAUUAACACCAUUCCGAUUGCCAUUAAUGAGGCAGAAU
ACGUCGGCCCUGGUCCCGGCGCCGCGUUCCAAGGAGCCCACGCACGAUUCGUGGCGGCAGCUG
CGGGACCCGGGCCUGGUGCCGGUUGGCUGGCUUUUUUCCGCGACCUGGUGGCCAGAGGACUUG
GACCCGGUCCGGGCGCCAGCAUUAUCAGGCUGGUCGGGGCCGUGCUGGCGGAACAGCAUGGAC
CUGGCCCGGGAAUGAGCUUCGUGACCACCCAACCUGAGGCGCUUGCCGCGGCGGCAGGCCCUG
GACCCGGGAUGCAUGUGUCUUUUGUGAUGGCCUACCCGGAAAUGCUGGCCGCGGGUCCGGGCC
CCGGAGCCUACGGAUCAUUCGUGCGCACUGUGUCCCUCCCCGUGGGAGCCGGACCUGGACCGG
GGUUGGAGAAUGAUAACCAGCUGCUCUACAACUAUCCGGGGGCCCUCGGCAUCGUGGCGGGCC
UCGCUGUCCUCGCCGUGGUGGUGAUCGGAGCCGUCGUCGCCGCUGUGAUGUGCCGCCGCAAGU
CCAGCGGCGGAAAGGGGGGCAGCUAUAGCCAGGCAGCCUGUUCCGACUCCGCGCAGGGGUCGG
AUGUGUCCUUGACCGCCUGAUGAUAAUAGGCUCGCUUUCUUGCUGUCCAAUUUCUAUUAAAGG
UUCCUUUGUUCCCUAAGUCCAACUACUAAACUGGGGGAUAUUAUGAAGGGCCUUGAGCAUCUG
GAUUCUGCCUAAUAAAAAACAUUUAUUUUCAUUGCAA.
An Mtb CD4 T cell-focused vaccine construct. Protein
sequence consisting of SEQ ID NO. 20 (associated with
Table 46) and the LAMP-1 signal sequences (underlined;
SEQ ID NOs. 21 and 22).
SEQ ID NO. 39
MAAPGSARRPLLLLLLLLLLGLMHCASAAEMKTDAATLAQEAGNFERISGDLKTQIDQVESTA
GSLQGQWRGAAGTAAQAAVVRFQEAANKQKQELDEISTNIRQAGVQYSRADEEQQQALSSQMG
FGPGPGVDFGALPPEINSARMYAGPGSASLVAAAQMWDSVASDLFSAASAFQSVVWGLTVGSW
IGSSAGLMVAAASPYVAWMSVTAGQAELTAAQVRVAAAAYETAYGLTVPPPVIAENRAELMIL
IATNLLGQNTPAIAVNEAEYGEMWAQDAAAMFGYAAATATATATLLPFEEAPEMTSAGGLLEQ
AAAVEEASDTAAANQLMNNVPQALQQLAQPTQGTTPSSKLGGLWKTVSPHRSPISNMVSMANN
HMGPGPGTEQQWNFAGIEAAASAIQGNVTSIHSLLDEGKQSLTKLAAAWGGSGSEAYQGVQQK
WDATATELNNALQNLARTISEAGQAMASTEGNVTGMFAGPGPGTSRFMTDPHAMRDMAGRFEV
HAQTVEDEARRMWASAQNISGAGWSGMAEATSLDTMTQMNQAFRNIVNMLHGVRDGLVRDANN
YEQQEQASQQILSSGPGPGSQIMYNYPAMLGHAGDMAGYAGTLQSLGAEIAVEQAALQSAWQG
DTGITYQAWQAQWNQAMEDLVRAYHAMSSTHEANTMAMMARDTAEAAKWGGGPGPGTINYQFG
DVDAHGAMIRAQAGSLEAEHQAIISDVLTASDFWGGAGSAACQGFITQLGRNFQVIYEQANAH
GQKVQAAGNNMAQTDSAVGSSWAGPGPGPVGGQSSFYSDWYSPACGKAGCQTYKWETFLTSEL
PQWLSANRAVKPTGSAAIGLSMAGSSAMILAAYHPQQFIYAGSLSALLDPSQGMGGPGPGAQI
YQAVSAQAAAIHGPGPGPSPSMGRDIKVQFQSGPGPGGINTIPIAINEAEYVGPGPGAAFQGA
HARFVAAAAGPGPGAGWLAFFRDLVARGLGPGPGASIIRLVGAVLAEQHGPGPGMSFVTTQPE
ALAAAAGPGPGMHVSFVMAYPEMLAAGPGPGAYGSFVRTVSLPVGAGPGPGLENDNQLLYNYP
GALLIPIAVGGALAGLVLIVLIAYLVGRKRSHAGYQTI.
Codon optimized forward nucleotide sequence
corresponding to the SEQ ID NO. 39 protein
sequence. The HBB UTRs are underlined. N1-
methylpseudouridine is substituted for all
uridines (U) during mRNA synthesis.
SEQ ID NO. 40
ACAUUUGCUUCUGACACAACUGUGUUCACUAGCAACCUCAAACAGCCACCAUGGCCGCUCCUG
GAUCCGCCCGCAGACCGUUGUUGCUCCUGUUACUACUGCUCUUACUUGGACUCAUGCACUGCG
CCUCCGCCGCUGAGAUGAAAACCGACGCCGCGACCCUGGCCCAGGAAGCCGGAAACUUCGAGC
GGAUUUCCGGAGAUCUUAAGACUCAGAUCGACCAGGUCGAAUCCACCGCCGGAUCGCUCCAGG
GACAGUGGAGAGGCGCCGCGGGAACCGCGGCCCAGGCCGCCGUCGUGCGGUUCCAAGAGGCCG
CCAACAAGCAGAAGCAGGAACUGGACGAAAUUUCCACUAACAUUCGCCAAGCUGGCGUGCAGU
ACUCGAGAGCCGAUGAAGAACAGCAGCAAGCCCUCUCCUCACAAAUGGGUUUCGGACCUGGGC
CCGGCGUGGACUUCGGAGCCCUGCCACCUGAAAUCAACUCCGCCCGGAUGUACGCAGGACCUG
GAUCCGCCAGCCUGGUGGCGGCCGCGCAGAUGUGGGAUAGCGUGGCCUCCGACCUGUUCAGCG
CGGCUUCAGCUUUCCAAAGCGUGGUCUGGGGACUGACUGUGGGAUCCUGGAUCGGAUCAAGCG
CAGGCCUCAUGGUGGCCGCGGCGUCCCCUUACGUGGCCUGGAUGAGCGUGACCGCCGGCCAGG
CAGAACUGACCGCAGCCCAAGUCCGCGUGGCCGCAGCCGCCUACGAGACUGCCUACGGUCUGA
CGGUGCCGCCGCCAGUGAUCGCCGAGAACAGAGCAGAGCUCAUGAUCCUCAUCGCGACCAACC
UACUGGGCCAGAACACUCCGGCGAUUGCCGUGAACGAAGCCGAAUAUGGGGAGAUGUGGGCUC
AGGACGCUGCAGCCAUGUUCGGAUAUGCGGCCGCGACUGCUACCGCCACCGCCACUCUGCUGC
CGUUCGAGGAGGCCCCCGAAAUGACUUCCGCGGGAGGCCUGUUGGAGCAAGCUGCCGCGGUGG
AGGAAGCUAGCGACACCGCCGCAGCCAACCAGCUCAUGAACAACGUGCCACAGGCCCUGCAGC
AGCUGGCCCAGCCCACUCAAGGGACCACCCCGAGCUCAAAGCUGGGCGGUCUGUGGAAAACCG
UGUCCCCCCACCGCUCGCCCAUUUCCAACAUGGUGUCAAUGGCGAACAACCACAUGGGUCCUG
GACCUGGAACGGAGCAGCAGUGGAACUUCGCCGGGAUCGAAGCCGCCGCCUCGGCUAUCCAAG
GGAAUGUCACCUCGAUCCAUUCCCUUCUGGACGAAGGAAAGCAGUCCCUGACCAAGCUUGCGG
CAGCCUGGGGCGGAAGCGGCAGCGAAGCCUACCAGGGCGUGCAGCAAAAAUGGGACGCAACCG
CCACUGAGCUGAACAACGCCCUCCAAAACCUGGCUAGAACUAUUUCCGAGGCCGGACAGGCUA
UGGCCAGCACCGAGGGCAACGUGACCGGGAUGUUCGCUGGCCCUGGGCCGGGCACCUCGCGGU
UCAUGACCGAUCCUCAUGCUAUGCGGGAUAUGGCUGGACGGUUCGAGGUGCACGCCCAAACUG
UGGAGGACGAGGCCCGCCGGAUGUGGGCCAGCGCGCAGAACAUCUCGGGGGCCGGCUGGUCCG
GAAUGGCCGAAGCCACCUCCCUCGAUACCAUGACCCAGAUGAACCAGGCCUUCCGGAACAUUG
UGAACAUGCUGCAUGGAGUGCGGGACGGCCUGGUCCGGGACGCCAACAAUUACGAGCAGCAGG
AGCAGGCCUCUCAGCAAAUCCUGUCCAGCGGGCCGGGGCCUGGAUCACAAAUCAUGUACAACU
ACCCGGCAAUGCUGGGCCACGCUGGAGACAUGGCCGGCUACGCCGGCACGCUGCAGUCACUCG
GUGCCGAGAUCGCCGUCGAGCAGGCGGCACUGCAGAGCGCUUGGCAGGGAGACACUGGCAUUA
CCUACCAAGCGUGGCAGGCUCAGUGGAAUCAAGCUAUGGAAGAUCUGGUCCGCGCGUACCACG
CCAUGUCCUCCACUCACGAAGCCAACACCAUGGCUAUGAUGGCCAGGGACACUGCCGAGGCAG
CCAAAUGGGGGGGAGGACCGGGGCCGGGAACCAUAAACUACCAGUUUGGCGACGUCGACGCCC
ACGGAGCCAUGAUCAGGGCGCAGGCCGGGUCGCUGGAAGCAGAACACCAGGCCAUCAUCUCCG
AUGUGCUGACCGCCUCCGACUUUUGGGGAGGAGCCGGUUCGGCUGCCUGCCAAGGGUUCAUCA
CACAAUUGGGAAGGAACUUCCAGGUCAUCUACGAGCAGGCCAAUGCACACGGUCAAAAGGUCC
AAGCGGCGGGCAACAACAUGGCCCAGACUGACUCGGCCGUGGGCAGCAGUUGGGCCGGUCCCG
GCCCUGGCCCUGUCGGGGGGCAGUCAUCCUUCUACUCCGACUGGUACUCCCCCGCCUGCGGAA
AGGCCGGCUGUCAGACCUAUAAGUGGGAAACCUUCCUGACCUCCGAACUCCCGCAGUGGCUCU
CGGCGAAUCGGGCAGUGAAGCCCACCGGGUCGGCGGCUAUUGGACUGUCCAUGGCCGGUUCCA
GCGCGAUGAUCCUGGCCGCCUAUCACCCCCAGCAAUUCAUCUACGCUGGCUCCCUGUCCGCCC
UGCUUGACCCAUCGCAGGGGAUGGGUGGACCGGGCCCUGGGGCCCAGAUCUACCAAGCAGUGU
CGGCCCAAGCCGCAGCGAUCCAUGGACCAGGACCGGGACCGUCCCCGAGCAUGGGCCGGGAUA
UUAAGGUUCAGUUCCAGUCCGGACCCGGCCCGGGGGGUAUUAACACCAUUCCGAUUGCCAUUA
AUGAGGCAGAAUACGUCGGCCCUGGUCCCGGCGCCGCGUUCCAAGGAGCCCACGCACGAUUCG
UGGCGGCAGCUGCGGGACCCGGGCCUGGUGCCGGUUGGCUGGCUUUUUUCCGCGACCUGGUGG
CCAGAGGACUUGGACCCGGUCCGGGCGCCAGCAUUAUCAGGCUGGUCGGGGCCGUGCUGGCGG
AACAGCAUGGACCUGGCCCGGGAAUGAGCUUCGUGACCACCCAACCUGAGGCGCUUGCCGCGG
CGGCAGGCCCUGGACCCGGGAUGCAUGUGUCUUUUGUGAUGGCCUACCCGGAAAUGCUGGCCG
CGGGUCCGGGCCCCGGAGCCUACGGAUCAUUCGUGCGCACUGUGUCCCUCCCCGUGGGAGCCG
GACCUGGACCGGGGUUGGAGAAUGAUAACCAGCUGCUCUACAACUAUCCGGGGGCCCUCCUGA
UUCCUAUCGCCGUGGGCGGUGCCUUGGCCGGCCUGGUGCUGAUCGUCCUGAUUGCCUAUCUCG
UGGGGAGGAAGAGAUCGCAUGCCGGCUACCAGACCAUCUGAUGAUAAUAGGCUCGCUUUCUUG
CUGUCCAAUUUCUAUUAAAGGUUCCUUUGUUCCCUAAGUCCAACUACUAAACUGGGGGAUAUU
AUGAAGGGCCUUGAGCAUCUGGAUUCUGCCUAAUAAAAAACAUUUAUUUUCAUUGCAA.
An Mtb CD4 T cell-focused vaccine construct. Protein
sequence consisting of SEQ ID NO. 20 (associated with
Table 46) and the HLA-DRa SP (underlined; SEQ ID NO. 29).
SEQ ID NO. 41
MAISGVPVLGFFIIAVLMSAQESWAAEMKTDAATLAQEAGNFERISGDLKTQIDQVESTAGSL
QGQWRGAAGTAAQAAVVRFQEAANKQKQELDEISTNIRQAGVQYSRADEEQQQALSSQMGFGP
GPGVDFGALPPEINSARMYAGPGSASLVAAAQMWDSVASDLFSAASAFQSVVWGLTVGSWIGS
SAGLMVAAASPYVAWMSVTAGQAELTAAQVRVAAAAYETAYGLTVPPPVIAENRAELMILIAT
NLLGQNTPAIAVNEAEYGEMWAQDAAAMFGYAAATATATATLLPFEEAPEMTSAGGLLEQAAA
VEEASDTAAANQLMNNVPQALQQLAQPTQGTTPSSKLGGLWKTVSPHRSPISNMVSMANNHMG
PGPGTEQQWNFAGIEAAASAIQGNVTSIHSLLDEGKQSLTKLAAAWGGSGSEAYQGVQQKWDA
TATELNNALQNLARTISEAGQAMASTEGNVTGMFAGPGPGTSRFMTDPHAMRDMAGRFEVHAQ
TVEDEARRMWASAQNISGAGWSGMAEATSLDTMTQMNQAFRNIVNMLHGVRDGLVRDANNYEQ
QEQASQQILSSGPGPGSQIMYNYPAMLGHAGDMAGYAGTLQSLGAEIAVEQAALQSAWQGDTG
ITYQAWQAQWNQAMEDLVRAYHAMSSTHEANTMAMMARDTAEAAKWGGGPGPGTINYQFGDVD
AHGAMIRAQAGSLEAEHQAIISDVLTASDFWGGAGSAACQGFITQLGRNFQVIYEQANAHGQK
VQAAGNNMAQTDSAVGSSWAGPGPGPVGGQSSFYSDWYSPACGKAGCQTYKWETFLTSELPQW
LSANRAVKPTGSAAIGLSMAGSSAMILAAYHPQQFIYAGSLSALLDPSQGMGGPGPGAQIYQA
VSAQAAAIHGPGPGPSPSMGRDIKVQFQSGPGPGGINTIPIAINEAEYVGPGPGAAFQGAHAR
FVAAAAGPGPGAGWLAFFRDLVARGLGPGPGASIIRLVGAVLAEQHGPGPGMSFVTTQPEALA
AAAGPGPGMHVSFVMAYPEMLAAGPGPGAYGSFVRTVSLPVGAGPGPGLENDNQLLYNYPGA
L.
Codon optimized forward nucleotide sequence
corresponding to the SEQ ID NO. 41 protein
sequence. The HBB UTRs are underlined. N1-
methylpseudouridine is substituted for all
uridines (U) during mRNA synthesis.
SEQ ID NO. 42
ACAUUUGCUUCUGACACAACUGUGUUCACUAGCAACCUCAAACAGCCACCAUGGCCAUCUCCG
GAGUGCCUGUGCUCGGUUUUUUCAUCAUCGCCGUGCUUAUGUCCGCCCAGGAAUCAUGGGCCG
CUGAGAUGAAAACCGACGCCGCGACCCUGGCCCAGGAAGCCGGAAACUUCGAGCGGAUUUCCG
GAGAUCUUAAGACUCAGAUCGACCAGGUCGAAUCCACCGCCGGAUCGCUCCAGGGACAGUGGA
GAGGCGCCGCGGGAACCGCGGCCCAGGCCGCCGUCGUGCGGUUCCAAGAGGCCGCCAACAAGC
AGAAGCAGGAACUGGACGAAAUUUCCACUAACAUUCGCCAAGCUGGCGUGCAGUACUCGAGAG
CCGAUGAAGAACAGCAGCAAGCCCUCUCCUCACAAAUGGGUUUCGGACCUGGGCCCGGCGUGG
ACUUCGGAGCCCUGCCACCUGAAAUCAACUCCGCCCGGAUGUACGCAGGACCUGGAUCCGCCA
GCCUGGUGGCGGCCGCGCAGAUGUGGGAUAGCGUGGCCUCCGACCUGUUCAGCGCGGCUUCAG
CUUUCCAAAGCGUGGUCUGGGGACUGACUGUGGGAUCCUGGAUCGGAUCAAGCGCAGGCCUCA
UGGUGGCCGCGGCGUCCCCUUACGUGGCCUGGAUGAGCGUGACCGCCGGCCAGGCAGAACUGA
CCGCAGCCCAAGUCCGCGUGGCCGCAGCCGCCUACGAGACUGCCUACGGUCUGACGGUGCCGC
CGCCAGUGAUCGCCGAGAACAGAGCAGAGCUCAUGAUCCUCAUCGCGACCAACCUACUGGGCC
AGAACACUCCGGCGAUUGCCGUGAACGAAGCCGAAUAUGGGGAGAUGUGGGCUCAGGACGCUG
CAGCCAUGUUCGGAUAUGCGGCCGCGACUGCUACCGCCACCGCCACUCUGCUGCCGUUCGAGG
AGGCCCCCGAAAUGACUUCCGCGGGAGGCCUGUUGGAGCAAGCUGCCGCGGUGGAGGAAGCUA
GCGACACCGCCGCAGCCAACCAGCUCAUGAACAACGUGCCACAGGCCCUGCAGCAGCUGGCCC
AGCCCACUCAAGGGACCACCCCGAGCUCAAAGCUGGGCGGUCUGUGGAAAACCGUGUCCCCCC
ACCGCUCGCCCAUUUCCAACAUGGUGUCAAUGGCGAACAACCACAUGGGUCCUGGACCUGGAA
CGGAGCAGCAGUGGAACUUCGCCGGGAUCGAAGCCGCCGCCUCGGCUAUCCAAGGGAAUGUCA
CCUCGAUCCAUUCCCUUCUGGACGAAGGAAAGCAGUCCCUGACCAAGCUUGCGGCAGCCUGGG
GCGGAAGCGGCAGCGAAGCCUACCAGGGCGUGCAGCAAAAAUGGGACGCAACCGCCACUGAGC
UGAACAACGCCCUCCAAAACCUGGCUAGAACUAUUUCCGAGGCCGGACAGGCUAUGGCCAGCA
CCGAGGGCAACGUGACCGGGAUGUUCGCUGGCCCUGGGCCGGGCACCUCGCGGUUCAUGACCG
AUCCUCAUGCUAUGCGGGAUAUGGCUGGACGGUUCGAGGUGCACGCCCAAACUGUGGAGGACG
AGGCCCGCCGGAUGUGGGCCAGCGCGCAGAACAUCUCGGGGGCCGGCUGGUCCGGAAUGGCCG
AAGCCACCUCCCUCGAUACCAUGACCCAGAUGAACCAGGCCUUCCGGAACAUUGUGAACAUGC
UGCAUGGAGUGCGGGACGGCCUGGUCCGGGACGCCAACAAUUACGAGCAGCAGGAGCAGGCCU
CUCAGCAAAUCCUGUCCAGCGGGCCGGGGCCUGGAUCACAAAUCAUGUACAACUACCCGGCAA
UGCUGGGCCACGCUGGAGACAUGGCCGGCUACGCCGGCACGCUGCAGUCACUCGGUGCCGAGA
UCGCCGUCGAGCAGGCGGCACUGCAGAGCGCUUGGCAGGGAGACACUGGCAUUACCUACCAAG
CGUGGCAGGCUCAGUGGAAUCAAGCUAUGGAAGAUCUGGUCCGCGCGUACCACGCCAUGUCCU
CCACUCACGAAGCCAACACCAUGGCUAUGAUGGCCAGGGACACUGCCGAGGCAGCCAAAUGGG
GGGGAGGACCGGGGCCGGGAACCAUAAACUACCAGUUUGGCGACGUCGACGCCCACGGAGCCA
UGAUCAGGGCGCAGGCCGGGUCGCUGGAAGCAGAACACCAGGCCAUCAUCUCCGAUGUGCUGA
CCGCCUCCGACUUUUGGGGAGGAGCCGGUUCGGCUGCCUGCCAAGGGUUCAUCACACAAUUGG
GAAGGAACUUCCAGGUCAUCUACGAGCAGGCCAAUGCACACGGUCAAAAGGUCCAAGCGGCGG
GCAACAACAUGGCCCAGACUGACUCGGCCGUGGGCAGCAGUUGGGCCGGUCCCGGCCCUGGCC
CUGUCGGGGGGCAGUCAUCCUUCUACUCCGACUGGUACUCCCCCGCCUGCGGAAAGGCCGGCU
GUCAGACCUAUAAGUGGGAAACCUUCCUGACCUCCGAACUCCCGCAGUGGCUCUCGGCGAAUC
GGGCAGUGAAGCCCACCGGGUCGGCGGCUAUUGGACUGUCCAUGGCCGGUUCCAGCGCGAUGA
UCCUGGCCGCCUAUCACCCCCAGCAAUUCAUCUACGCUGGCUCCCUGUCCGCCCUGCUUGACC
CAUCGCAGGGGAUGGGUGGACCGGGCCCUGGGGCCCAGAUCUACCAAGCAGUGUCGGCCCAAG
CCGCAGCGAUCCAUGGACCAGGACCGGGACCGUCCCCGAGCAUGGGCCGGGAUAUUAAGGUUC
AGUUCCAGUCCGGACCCGGCCCGGGGGGUAUUAACACCAUUCCGAUUGCCAUUAAUGAGGCAG
AAUACGUCGGCCCUGGUCCCGGCGCCGCGUUCCAAGGAGCCCACGCACGAUUCGUGGCGGCAG
CUGCGGGACCCGGGCCUGGUGCCGGUUGGCUGGCUUUUUUCCGCGACCUGGUGGCCAGAGGAC
UUGGACCCGGUCCGGGCGCCAGCAUUAUCAGGCUGGUCGGGGCCGUGCUGGCGGAACAGCAUG
GACCUGGCCCGGGAAUGAGCUUCGUGACCACCCAACCUGAGGCGCUUGCCGCGGCGGCAGGCC
CUGGACCCGGGAUGCAUGUGUCUUUUGUGAUGGCCUACCCGGAAAUGCUGGCCGCGGGUCCGG
GCCCCGGAGCCUACGGAUCAUUCGUGCGCACUGUGUCCCUCCCCGUGGGAGCCGGACCUGGAC
CGGGGUUGGAGAAUGAUAACCAGCUGCUCUACAACUAUCCGGGGGCCCUCUGAUGAUAAUAGG
CUCGCUUUCUUGCUGUCCAAUUUCUAUUAAAGGUUCCUUUGUUCCCUAAGUCCAACUACUAAA
CUGGGGGAUAUUAUGAAGGGCCUUGAGCAUCUGGAUUCUGCCUAAUAAAAAACAUUUAUUUUC
AUUGCAA.
An Mtb CD4 T cell-focused vaccine construct. Protein
sequence consisting of SEQ ID NO. 20 (associated with
Table 46) and the tPA (underlined; SEQ ID NO. 30).
SEQ ID 43
MDAMKRGLCCVLLLCGAVFVSAAEMKTDAATLAQEAGNFERISGDLKTQIDQVESTAGSLQGQ
WRGAAGTAAQAAVVRFQEAANKQKQELDEISTNIRQAGVQYSRADEEQQQALSSQMGFGPGPG
VDFGALPPEINSARMYAGPGSASLVAAAQMWDSVASDLFSAASAFQSVVWGLTVGSWIGSSAG
LMVAAASPYVAWMSVTAGQAELTAAQVRVAAAAYETAYGLTVPPPVIAENRAELMILIATNLL
GQNTPAIAVNEAEYGEMWAQDAAAMFGYAAATATATATLLPFEEAPEMTSAGGLLEQAAAVEE
ASDTAAANQLMNNVPQALQQLAQPTQGTTPSSKLGGLWKTVSPHRSPISNMVSMANNHMGPGP
GTEQQWNFAGIEAAASAIQGNVTSIHSLLDEGKQSLTKLAAAWGGSGSEAYQGVQQKWDATAT
ELNNALQNLARTISEAGQAMASTEGNVTGMFAGPGPGTSRFMTDPHAMRDMAGRFEVHAQTVE
DEARRMWASAQNISGAGWSGMAEATSLDTMTQMNQAFRNIVNMLHGVRDGLVRDANNYEQQEQ
ASQQILSSGPGPGSQIMYNYPAMLGHAGDMAGYAGTLQSLGAEIAVEQAALQSAWQGDTGITY
QAWQAQWNQAMEDLVRAYHAMSSTHEANTMAMMARDTAEAAKWGGGPGPGTINYQFGDVDAHG
AMIRAQAGSLEAEHQAIISDVLTASDFWGGAGSAACQGFITQLGRNFQVIYEQANAHGQKVQA
AGNNMAQTDSAVGSSWAGPGPGPVGGQSSFYSDWYSPACGKAGCQTYKWETFLTSELPQWLSA
NRAVKPTGSAAIGLSMAGSSAMILAAYHPQQFIYAGSLSALLDPSQGMGGPGPGAQIYQAVSA
QAAAIHGPGPGPSPSMGRDIKVQFQSGPGPGGINTIPIAINEAEYVGPGPGAAFQGAHARFVA
AAAGPGPGAGWLAFFRDLVARGLGPGPGASIIRLVGAVLAEQHGPGPGMSFVTTQPEALAAAA
GPGPGMHVSFVMAYPEMLAAGPGPGAYGSFVRTVSLPVGAGPGPGLENDNQLLYNYPGAL.
Codon optimized forward nucleotide sequence
corresponding to the SEQ ID NO. 43 protein
sequence. The HBB UTRs are underlined. N1-
methylpseudouridine is substituted for all
uridines (U) during mRNA synthesis.
SEQ ID 44
ACAUUUGCUUCUGACACAACUGUGUUCACUAGCAACCUCAAACAGCCACCAUGGAUGCUAUGA
AGAGAGGAUUGUGUUGCGUGUUACUACUGUGCGGAGCCGUGUUUGUGUCCGCCGCUGAGAUGA
AAACCGACGCCGCGACCCUGGCCCAGGAAGCCGGAAACUUCGAGCGGAUUUCCGGAGAUCUUA
AGACUCAGAUCGACCAGGUCGAAUCCACCGCCGGAUCGCUCCAGGGACAGUGGAGAGGCGCCG
CGGGAACCGCGGCCCAGGCCGCCGUCGUGCGGUUCCAAGAGGCCGCCAACAAGCAGAAGCAGG
AACUGGACGAAAUUUCCACUAACAUUCGCCAAGCUGGCGUGCAGUACUCGAGAGCCGAUGAAG
AACAGCAGCAAGCCCUCUCCUCACAAAUGGGUUUCGGACCUGGGCCCGGCGUGGACUUCGGAG
CCCUGCCACCUGAAAUCAACUCCGCCCGGAUGUACGCAGGACCUGGAUCCGCCAGCCUGGUGG
CGGCCGCGCAGAUGUGGGAUAGCGUGGCCUCCGACCUGUUCAGCGCGGCUUCAGCUUUCCAAA
GCGUGGUCUGGGGACUGACUGUGGGAUCCUGGAUCGGAUCAAGCGCAGGCCUCAUGGUGGCCG
CGGCGUCCCCUUACGUGGCCUGGAUGAGCGUGACCGCCGGCCAGGCAGAACUGACCGCAGCCC
AAGUCCGCGUGGCCGCAGCCGCCUACGAGACUGCCUACGGUCUGACGGUGCCGCCGCCAGUGA
UCGCCGAGAACAGAGCAGAGCUCAUGAUCCUCAUCGCGACCAACCUACUGGGCCAGAACACUC
CGGCGAUUGCCGUGAACGAAGCCGAAUAUGGGGAGAUGUGGGCUCAGGACGCUGCAGCCAUGU
UCGGAUAUGCGGCCGCGACUGCUACCGCCACCGCCACUCUGCUGCCGUUCGAGGAGGCCCCCG
AAAUGACUUCCGCGGGAGGCCUGUUGGAGCAAGCUGCCGCGGUGGAGGAAGCUAGCGACACCG
CCGCAGCCAACCAGCUCAUGAACAACGUGCCACAGGCCCUGCAGCAGCUGGCCCAGCCCACUC
AAGGGACCACCCCGAGCUCAAAGCUGGGCGGUCUGUGGAAAACCGUGUCCCCCCACCGCUCGC
CCAUUUCCAACAUGGUGUCAAUGGCGAACAACCACAUGGGUCCUGGACCUGGAACGGAGCAGC
AGUGGAACUUCGCCGGGAUCGAAGCCGCCGCCUCGGCUAUCCAAGGGAAUGUCACCUCGAUCC
AUUCCCUUCUGGACGAAGGAAAGCAGUCCCUGACCAAGCUUGCGGCAGCCUGGGGCGGAAGCG
GCAGCGAAGCCUACCAGGGCGUGCAGCAAAAAUGGGACGCAACCGCCACUGAGCUGAACAACG
CCCUCCAAAACCUGGCUAGAACUAUUUCCGAGGCCGGACAGGCUAUGGCCAGCACCGAGGGCA
ACGUGACCGGGAUGUUCGCUGGCCCUGGGCCGGGCACCUCGCGGUUCAUGACCGAUCCUCAUG
CUAUGCGGGAUAUGGCUGGACGGUUCGAGGUGCACGCCCAAACUGUGGAGGACGAGGCCCGCC
GGAUGUGGGCCAGCGCGCAGAACAUCUCGGGGGCCGGCUGGUCCGGAAUGGCCGAAGCCACCU
CCCUCGAUACCAUGACCCAGAUGAACCAGGCCUUCCGGAACAUUGUGAACAUGCUGCAUGGAG
UGCGGGACGGCCUGGUCCGGGACGCCAACAAUUACGAGCAGCAGGAGCAGGCCUCUCAGCAAA
UCCUGUCCAGCGGGCCGGGGCCUGGAUCACAAAUCAUGUACAACUACCCGGCAAUGCUGGGCC
ACGCUGGAGACAUGGCCGGCUACGCCGGCACGCUGCAGUCACUCGGUGCCGAGAUCGCCGUCG
AGCAGGCGGCACUGCAGAGCGCUUGGCAGGGAGACACUGGCAUUACCUACCAAGCGUGGCAGG
CUCAGUGGAAUCAAGCUAUGGAAGAUCUGGUCCGCGCGUACCACGCCAUGUCCUCCACUCACG
AAGCCAACACCAUGGCUAUGAUGGCCAGGGACACUGCCGAGGCAGCCAAAUGGGGGGGAGGAC
CGGGGCCGGGAACCAUAAACUACCAGUUUGGCGACGUCGACGCCCACGGAGCCAUGAUCAGGG
CGCAGGCCGGGUCGCUGGAAGCAGAACACCAGGCCAUCAUCUCCGAUGUGCUGACCGCCUCCG
ACUUUUGGGGAGGAGCCGGUUCGGCUGCCUGCCAAGGGUUCAUCACACAAUUGGGAAGGAACU
UCCAGGUCAUCUACGAGCAGGCCAAUGCACACGGUCAAAAGGUCCAAGCGGCGGGCAACAACA
UGGCCCAGACUGACUCGGCCGUGGGCAGCAGUUGGGCCGGUCCCGGCCCUGGCCCUGUCGGGG
GGCAGUCAUCCUUCUACUCCGACUGGUACUCCCCCGCCUGCGGAAAGGCCGGCUGUCAGACCU
AUAAGUGGGAAACCUUCCUGACCUCCGAACUCCCGCAGUGGCUCUCGGCGAAUCGGGCAGUGA
AGCCCACCGGGUCGGCGGCUAUUGGACUGUCCAUGGCCGGUUCCAGCGCGAUGAUCCUGGCCG
CCUAUCACCCCCAGCAAUUCAUCUACGCUGGCUCCCUGUCCGCCCUGCUUGACCCAUCGCAGG
GGAUGGGUGGACCGGGCCCUGGGGCCCAGAUCUACCAAGCAGUGUCGGCCCAAGCCGCAGCGA
UCCAUGGACCAGGACCGGGACCGUCCCCGAGCAUGGGCCGGGAUAUUAAGGUUCAGUUCCAGU
CCGGACCCGGCCCGGGGGGUAUUAACACCAUUCCGAUUGCCAUUAAUGAGGCAGAAUACGUCG
GCCCUGGUCCCGGCGCCGCGUUCCAAGGAGCCCACGCACGAUUCGUGGCGGCAGCUGCGGGAC
CCGGGCCUGGUGCCGGUUGGCUGGCUUUUUUCCGCGACCUGGUGGCCAGAGGACUUGGACCCG
GUCCGGGCGCCAGCAUUAUCAGGCUGGUCGGGGCCGUGCUGGCGGAACAGCAUGGACCUGGCC
CGGGAAUGAGCUUCGUGACCACCCAACCUGAGGCGCUUGCCGCGGCGGCAGGCCCUGGACCCG
GGAUGCAUGUGUCUUUUGUGAUGGCCUACCCGGAAAUGCUGGCCGCGGGUCCGGGCCCCGGAG
CCUACGGAUCAUUCGUGCGCACUGUGUCCCUCCCCGUGGGAGCCGGACCUGGACCGGGGUUGG
AGAAUGAUAACCAGCUGCUCUACAACUAUCCGGGGGCCCUCUGAUGAUAAUAGGCUCGCUUUC
UUGCUGUCCAAUUUCUAUUAAAGGUUCCUUUGUUCCCUAAGUCCAACUACUAAACUGGGGGAU
AUUAUGAAGGGCCUUGAGCAUCUGGAUUCUGCCUAAUAAAAAACAUUUAUUUUCAUUGCAA.

### Example 33: HLA Class I CD8 T Cell-Focused TB mRNA Vaccines

Hypotheses for antigen selection for CD8 T cell-focused mRNA cassettes. The goal was to create an mRNA vaccine coding for HLA class I epitopes. We took two different approaches to selecting antigens:
1. Revaccination with BCG has provided mixed results in the prevention of disease. Therefore, one hypothesis is that BCG immunization negatively influences protective CD8 T cell responses, and that inducing CD8 T cell responses to epitopes unique to Mtb would broaden the memory T cell pool and improve prevention of disease. This approach is termed the “Mtb-only” HLA class I mRNA vaccine.
2. Alternatively, BCG vaccination may just be a poor inducer of protective CD8 T cell immunity. Therefore, a hypothesis is that mRNA vaccination against epitopes both shared between Mtb/BCG or unique to Mtb would boost existing BCG-induced memory and broaden the CD8 T cell memory pool to new specificities. The approach is termed the “Mixed” HLA class I mRNA cassette.

Selection of HLA class I epitopes for the “Mtb-only” vaccine. Mtb open reading frames (ORFs) previously shown to be immunogenic and not expressed by BCG were selected identified (Table 47, associated SEQ ID NOs. 45-54). Using the Mtb strain H37Rv as the reference sequence (taxonomy ID 83332, NCBI:txid83332), N-terminus signal peptides were removed from ORFs (mpt64, mpt70 and mpt83) and areas of homology to BCG within PPE68 were removed (Okkels et al. (2003) Curr Pharm Biotechnol 4, 69-83). Using the publicly available class I epitope prediction algorithm NetMHCpan EL 4.1 accessed through the Immune Epitope Database and Analysis Resource (www.IEDB.org), MHC class I binding predictions for peptides of 8, 9 or 10 amino acids (aa) in length were made for 73 globally prevalent HLA class I alleles (Nilsson et al. (2021) Front Immunol 12, 728936). Only high-affinity epitope predictions with a percentile rank of ≤0.5 were kept, which captures >80% of natural epitopes with high specificity (Jurtz et al. (2017) J Immunol 99, 3360-3368; Peters et al. (2020) Annu Rev Immunol 38, 123-145).

As the NetMHCpan EL output was too large to capture in an mRNA cassette, predicted high affinity epitopes were filtered using the PopCover-2.0 algorithm (Nilsson et al. (2021) Front Immunol 12, 728936, services.healthtech.dtu.dk/service.php?PopCover-2.0). PopCover-2.0 reduces an input dataset of predicted epitopes by removing sequence redundancies, and then it selects a user-defined number of epitopes that achieve the broadest population HLA allele coverage. The following were input into the PopCover-2.0 algorithm: 1) the list of predicted epitopes and their HLA class I restrictions with percentile rank ≤0.5, 2) the complete Mtb ORF protein sequences corresponding to the epitopes, and 3) the list of 73 globally prevalent HLA class I alleles and there population frequencies. The number of output epitopes was set to 200 and the length of peptides to extract from the ORF protein sequences was set to 10. The PopCover-2.0 output of the top 200 epitopes were predicted to cover 86.98% HLA-A loci, 83.85% HLA-B loci, 87.16% HLA-C loci, and 99.76% coverage across all HLA class I loci. These 200 epitopes were then sorted by Mtb gene and manually curated to identify epitopes that were adjacent/overlapping, which were then concatenated into longer peptides. With a target length of 3000-3300 nucleotides (1000-1100 aa) for the mRNA cassette, the top PopCover-2.0 ranked peptides from each Mtb ORF were prioritized for inclusion in the cassette (Table 48, SEQ ID NOs. 106-137).

Selection of HLA class I epitopes for the “Mixed” vaccine. ORFs demonstrated to be immunogenic to either CD4 and/or CD8 T cells were selected (Woodworth (2021) Nat Comm 12, 6658; Okkels et al. (2003) Curr Pharm Biotechnol 4, 69-83; Coppola et al. (2021) NPJ Vaccines 6, 81; Sali et al. (2014) Vaccine 32, 4051-4058; Arlehamn et al. (2013) PLoS Pathog 9, e1003130; Arlehamn et al. (2016) PLoS Pathog 12, e1005760) (see Table 49, associated SEQ ID NOs. 55-85). Same as for the “Mtb-only” cassette, the complete ORF protein sequences (H37Rv reference strain) were input into the NetMHC pan EL 4.1 algorithm accessed through IEDB. MHC class I binding predictions for peptides of 8, 9 or 10 amino acids (aa) in length were made for 73 globally prevalent HLA class I alleles and only high-affinity epitope predictions with a percentile rank of ≤0.5 were kept. The PopCover-2.0 algorithm was then used to identify a subset of epitopes that would provide the population broadest HLA allele coverage. The top 200 epitopes were predicted to cover 86.98% HLA-A loci, 83.85% HLA-B loci, 87.16% HLA-C loci, and 99.73% coverage across all HLA class I loci. These epitopes were then sorted by Mtb gene and manually curated to identify epitopes that were adjacent/overlapping, which were then concatenated into longer peptides. With a target length of 3000-3300 nucleotides (1000-1100 aa) for the mRNA cassette, the top PopCover-2.0 ranked peptides were prioritized for inclusion in the cassette (Table 50, SEQ ID NOs 138-203).

Optimization of the peptide order to decrease the formation of junctional epitopes. To vaccinate against multiple T cell epitopes, it is not feasible to manufacture and formulate individual mRNA-LNPs encoding each separately, especially when many epitopes are being targeted. Consequently, epitopes must be combined into a single ORF that, upon transfection of immune cells, is translated into a synthetic protein. To be presented on MHC class I molecules to CD8 T cells, cytosolic proteins must be processed into short peptide fragments by the proteosome, further trimmed by peptidases, loaded onto nascent MHC class I molecules in the endoplasmic reticulum and presented on the cell surface to T cells (Yewdell and Bennink (2001) Curr Opin Immunol 13, 13-18). Thus, the vaccinal protein will be cleaved into short peptides comprised of a mixture of the intended pathogen-specific epitopes as well as peptides that span a synthetic junction between two tandem epitopes. These junctional epitopes, or “neo-epitopes”, have the potential to bind to class I molecules and prime irrelevant CD8 T cell responses. These neo-epitopes can detract from the intended vaccine epitopes by competing for binding to HLA molecules or by generating unwanted immunodominant T cell responses, thus decreasing the immunogenicity of the vaccine (Livingston et al. (2002) J Immunol 168, 5499-5506).

To prevent the formation of neo-epitopes in polyepitope vaccines, non-immunogenic repetitive GP (gly-pro) and GS (gly-ser) spacers have been identified that can broadly prevent MHC class II-restricted junctional epitopes ((Livingston et al. (2002) J Immunol 168, 5499-5506; Sahin et al (2017) Nature 547, 222-226), and spacers such as AAY (ala-ala-tyr) have been identified for MHC class I-restricted epitopes to promote C-terminal proteasomal cleavage between linked epitopes (Wang et al. (2004) Scan J Immunol 60, 219-225; Velders et al. (2001) J Immunol 166, 5366-5373; Schubert et al. (2016) Genome Med 8, 9). Studies incorporating AAY linkers between poly-epitope DNA vaccine constructs demonstrated improved CD8 T cell priming (Wang et al. (2004) Scan J Immunol 60, 219-225; Velders et al. (2001) J Immunol 166, 5366-5373) and protection against tumor growth in mice (Velders et al. (2001) J Immunol 166, 5366-5373); however, these studies did not address if the inclusion of AAY linkers created junctional neo-epitopes. Computational analysis of multi-epitope vaccine designs indicated that fixed linkers such as AAY would increase junctional epitopes (Schubert et al. (2016) Genome Med 8, 9). Indeed, when AAY was inserted between multiple class I peptides from Table 48 and epitopes were predicted using the NetMHCpan EL algorithm, many high affinity junctional epitopes incorporating AAY were predicted (unpublished observations). This is likely because MHC binding has been shown to be far more selective than TAP transport or proteasomal processing in predicting class I ligands (Annu Rev Immunol, 2020 Apr. 26:38:123-145; PMID: 32045313). The studies demonstrating experimental benefit to including AAY linkers (Schubert et al. (2016) Genome Med 8, 9; Peters et al. (2020) Annu Rev Immunol 38, 123-145) incorporated a small number of T cell epitopes (<10) where the chance that neo-epitopes could detract from the intended epitopes was theoretically small relative to a vaccine incorporating many more epitopes.

To decrease the probability of irrelevant MHC class I-restricted junctional epitopes and avoid the use of linkers, we devised a method to optimize the order of epitopes in an mRNA cassette that generates as few predicted class I junctional neo-epitopes as possible. To clarify terminology, input protein sequences identified using the combination of NetMHCpan EL 4.1 and PopCover-2.0 will herein be referred to as peptides, as these can be minimal 9-10mer peptides or longer, concatenated sequences or even entire ORFs; the term epitopes will refer to the minimal MHC ligand. The first step was to predict junctional epitopes between all two juxtaposed peptides. All pairwise combinations (excluding self-pairings) between the last 8 aa of each peptide and first 8 aa of each peptide were made. Since the majority of class I epitopes are 9 aa long (Trolle et al. (2016) J Immunol 196, 1480-1487; Gfeller et al., (2018) J Immunol 201, 3705-3716), any predicted 9mer epitope would have to incorporate at least one junctional aa, and therefore has the potential to be a unique T cell ligand. These 16 aa sequences were input into NetMHCpan EL 4.1 via IEDB and all 9mer class I ligands to a set of 27 globally common HLA-A and B alleles (Weiskopf et al. (2013) Proc Natl Acad Sci 110, E2046-2053) were predicted; only ligands with a percentile rank ≤2 were kept. For each predicted junctional epitope, the inverse of the percentile rank was used as a score to penalize that epitope (e.g. a percentile rank of 0.01 is given a score of 100). For each peptide pairing, the basic sum score for that junction was the sum of scores for all predicted epitopes (referred to as the unpenalized scoring function). A weighted penalized score added 5 for each predicted epitope that had a percentile rank <0.5, which captures the highest affinity ligands with the most accuracy (referred to as the penalized scoring function). The pairwise scores were interpreted as distance to generate a distance matrix covering all pairs of peptide junctions.

The problem of ordering the peptides to minimize the cumulative score of their junctions can be interpreted as a traveling salesman problem (TSP), where the peptides are cities and the junction scores are distances between cities. We used the “TSP” package available for R to calculate solutions for this problem. We added a dummy junction/city with distance zero to all others and specified it as the start location. This ensures that each peptide is only used once. For each of three algorithms (“nearest_insertion”, “farthest_insertion”, “cheapest_insertion”) available in the TSP package, and for each scoring function (penalized and unpenalized) we generated 100 random solutions using the “two-opt” method to improve any solutions. For each scoring function, we filtered the top 10 solutions by score, regardless of algorithm used. Using a greedy algorithm, we then selected 5 solutions out of each 10 that minimized similarity in the junctions used: starting with the lowest scoring solution, we iteratively added an additional solution from the top 10 having the least number of junctions overlapping with any already included solution until we had 5 for each scoring function. The top 5 solutions for each scoring function were then converted back to amino acid sequences and were examined by hand to select for experimental testing.

Optimizing peptide order for the “Mtb-only” HLA class I cassette. Table 51 shows the top 5 solutions with Basic Scoring (unpenalized scoring function) and top 5 using the weighted penalized scoring function. Table 52 shows the sequence of peptides (see Table 48 for the amino acid sequences) for the optimal solutions. SEQ ID NOs. 86-95 are the assembled strings of peptides that are associated with each solution.

Optimizing the peptide order for the “Mixed” HLA class I cassette. Table 53 shows the top 5 solutions with Basic Scoring (unpenalized scoring function) and top 5 using the weighted penalized scoring function. Table 54 shows the sequence of peptides (see Table 50 for the amino acid sequences) for the optimal solutions. SEQ ID NOs. 96-105 are the assembled strings of peptides that are associated with each solution.

Strategies of proteasomal targeting of vaccine antigens to enhance CD8 T cell priming. As described for the CD4 T cell-based mRNA vaccine, signal sequences that target proteins to the endosomal/lysosomal compartment support antigen processing and presentation by both MHC class I and class II pathways. However, for cytoplasmic proteins the canonical pathway for class I presentation is via the proteosome. Proteins are cleaved by the proteosome, shuttled into the ER where they bind class I molecules, and these complexes are shuttled to the cell surface for presentation to CD8 T cells. Strategies have been developed for nucleotide-based vaccines that promote this pathway of antigen presentation at the expense of CD4 T cell and B cell responses. One such approach is to covalently link ubiquitin to the N- or C-terminus of a protein. The ubiquitin pathway is an important method for tagging proteins for proteasomal destruction (Glickman and Ciechanover (2002) Physiol Rev 82, 373-428). Proteins are mono-ubiquitinated by the covalent attachment of the C-terminal G76 residue to target lysines. This ubiquitin serves as a docking station for additional attachment of ubiquitin molecules that results in the poly-ubiquitination of proteins and targeting to the proteasome. This process is reversible, and deubiquitinating enzymes cleave covalently attached mono-ubiquitin at residue G76. To prevent cleavage and promote poly-ubiquitin tagging of proteins and subsequent proteolysis, mutation of G to A in position 76 drastically decreases cleavage of the 76aa ubiquitin and causes poly-ubiquitination and rapid proteasomal processing (Rodriguez et al. (1997) J Virol 71, 8497-8503) (SEQ ID NO. 204). This approach has been used in DNA vaccines to enhance the generation of CD8 T cell responses (Rodriguez et al. (1997) J Virol 71, 8497-8503; Delogu et al. (2000) Infect Immun 68, 3097-3102; Delogu et al. (2002) Infect Immun 70, 293-302; Li et al. (2021) Genome Med 13, 56). Another published method of destabilizing ubiquitin to promote rapid targeting of proteins to the proteosome is to preserve the glycine at residue 76 and add an arginine directly afterwards (G76GR) (Delogu et al. (2000) Infect Immun 68, 3097-3102) (SEQ ID NO. 205).

Another approach to target cytosolic proteins to the proteosome is to coopt the IκBα destruction motif that regulates the degradation of this protein by specific ubiquitin ligases (Yaron et al. (1997) EMBO J 16m 6486-6494; Chen et al. (1995) Genes Dev 9, 1586-1597). The IκBα destruction motif can be attached to the N-terminus of a protein to promote proteasomal destruction and enhanced class I presentation to CD8 T cells (see SEQ ID NO. 206).

Integration of antigenic proteins and proteasomal targeting into CD8 T cell-focused mRNA vaccine cassettes. One example of an mRNA cassette uses an “Mtb-only” optimized polyepitope protein (SEQ ID NO. 91) and the G76A ubiquitin attached to the N-terminus (SEQ ID NO. 207). Another example uses a different “Mtb-only” optimized polyepitope protein (SEQ ID NO. 92) and the N-terminal G76A ubiquitin (SEQ ID NO. 208). One example of an mRNA cassette uses an “Mixed” optimized polyepitope protein (SEQ ID NO. 101) and the G76A ubiquitin attached to the N-terminus (SEQ ID NO. 209). Another example uses a different “Mixed” optimized polyepitope protein (SEQ ID NO. 102) and the N-terminal G76A ubiquitin (SEQ ID NO. 210).

TABLE 47
List of Mtb-specific ORFs not expressed by BCG that were
used as input for prediction of HLA class I epitopes to
globally prevalent HLA alleles. References refer to publications
identifying these proteins as immunogenic.

| | Protein | Amino acids | | Reference |
|---|---|---|---|---|
| Gene | name | used | Notes | (PMID) |
| Rv1980c | mpt64 | 24-228 | Signal peptide (aa 1-23) | 34083546, |
| | | | removed | 24886957 |
| Rv2875 | mpt70 | 31-193 | Signal peptide (aa 1-30) | 34795205 |
| | | | removed | |
| Rv2873 | mpt83 | 31-220 | Signal peptide (aa 1-30) | 34795205 |
| | | | removed | |
| Rv3873 | PPE68 | 155-219, | Regions w/ homology to | 12570683, |
| | | 242-305 | BCG removed | 34795205 |
| Rv3876 | espI | 1-666 | No signal peptide | 34795205 |
| Rv3615c | espC | 1-103 | No signal peptide | 34795205, |
| | | | | 23358848 |
| Rv3616c | espA | 1-392 | No signal peptide | 34795205, |
| | | | | 34083546 |
| Rv3614c | espD | 1-184 | No signal peptide | 32887748 |
| Rv3881c | espB | 1-460 | No signal peptide | 32887748 |

| Reference (PMID) | |
|---|---|
| 34083546 | NPJ Vaccines. 2021 Jun. 3; 6(1): 81. |
| 24886957 | Vaccine. 2014 Jul. 7; 32(32): 4051-8. |
| 34795205 | Nat Commun. 2021 Nov. 18; 12(1): 6658. |
| 12570683 | Curr Pharm Biotechnol. 2003 February; 4(1): 69-83. |
| 23358848 | PLoS Pathog. 2013 January; 9(1): e1003130. |
| 32887748 | J Immunol. 2020 Oct. 15; 205(8): 2146-2155 |

TABLE 48
Peptides curated from the PopCover-2.0 output selected for
the “Mtb-only” mRNA cassette

| peptide | SEQ ID | | | amino acid sequence included |
|---|---|---|---|---|
| # | NO. | gene | protein | in cassette |
| 1 | SEQ ID | Rv1980c | Mpt64 | APKTYCEELKGTDTGQACQIQMSDPAYNINI |
| | NO. 106 | | | SLPSYYPDQKSLENYIAQTRDKFLSAATSST |
| | | | | PREAPYELNITSATYQSAIPPRGTQAVVLKV |
| | | | | YQNAGGTHPTTTYKAFDWDQAYRKPITYDTL |
| | | | | WQADTDPLPVVFPIVQGELSKQTGQQVSIAP |
| | | | | NAGLDPVNYQNFAVTNDGVIFFFNPGELLPE |
| | | | | AAGPTQVLVPRSAIDSMLA |
| 2 | SEQ ID | Rv2875 | Mpt70 | AVAASNNPELTTLTAALSGQLNPQVNLVDTL |
| | NO. 107 | | | NSGQYTVFAPTNAAFSKLPASTIDELKTNSS |
| | | | | LLTSILTY |
| 3 | SEQ ID | Rv2875 | Mpt70 | GVSTANATVYMIDSVLM |
| | NO. 108 | | | |
| 4 | SEQ ID | Rv2873 | Mpt83 | PAAPVTTAAMADPAADLI |
| | NO. 109 | | | |
| 5 | SEQ ID | Rv2873 | Mpt83 | GKLNPDVNLVDTLNGGEYTVFAPTNAAFDKL |
| | NO. 110 | | | PAATIDQLKTDAKLLSSILTYHVIAGQASPS |
| | | | | RIDGTHQTL |
| 6 | SEQ ID | Rv2873 | Mpt83 | GVHTANATVYMIDTVLM |
| | NO. 111 | | | |
| 7 | SEQ ID | Rv3873 | PPE68 | QAETAVNTLFEKLEPMA |
| | NO. 112 | | | |
| 8 | SEQ ID | Rv3873 | PPE68 | QLPPAATQTL |
| | NO. 113 | | | |
| 9 | SEQ ID | Rv3873 | PPE68 | TRTPLMSQLI |
| | NO. 114 | | | |
| 10 | SEQ ID | Rv3876 | EspI | MAADYDKLFR |
| | NO. 115 | | | |
| 11 | SEQ ID | Rv3876 | EspI | QPFFDPSASFP |
| | NO. 116 | | | |
| 12 | SEQ ID | Rv3876 | EspI | AASKPPTPPM |
| | NO. 117 | | | |
| 13 | SEQ ID | Rv3876 | EspI | RRTAPAPPWAKM |
| | NO. 118 | | | |
| 14 | SEQ ID | Rv3876 | EspI | WRHWVHALTRINLGLSPDEKYELDLHARVRR |
| | NO. 119 | | | NPRGSYQIAVVGL |
| 15 | SEQ ID | Rv3876 | EspI | QVRADRILAL |
| | NO. 120 | | | |
| 16 | SEQ ID | Rv3876 | EspI | ATIADVLAEKELSHYNDIRAHTSVNAVNLEV |
| | NO. 121 | | | LPAPEYSSAQRALSDADWHFIADPASRFYNL |
| | | | | VLA |
| 17 | SEQ ID | Rv3876 | EspI | FFDPLTRGVL |
| | NO. 122 | | | |
| 18 | SEQ ID | Rv3876 | EspI | NHIMPGEPNVAVKDLVRHFEQQVQPGRVVVM |
| | NO. 123 | | | PWDRHIAAGTEISLDLLDPIYKRKVLELAAA |
| | | | | L |
| 19 | SEQ ID | Rv3615c | EspC | SQFNDTLNVYLTAHNALGSSLHTAGVDLAK |
| | NO. 124 | | | |
| 20 | SEQ ID | Rv3616c | EspA | FIIDPTISAIDGLYDLLGIGIPNQGGILYSS |
| | NO. 125 | | | LEYFEKALEELA |
| 21 | SEQ ID | Rv3616c | EspA | FVRPVAVDLTYIPVVGHALSA |
| | NO. 126 | | | |
| 22 | SEQ ID | Rv3616c | EspA | MAVVGGALAYLVVKTLINATQLLKLLAKL |
| | NO. 127 | | | |
| 23 | SEQ ID | Rv3616c | EspA | VHAASTRQALR |
| | NO. 128 | | | |
| 24 | SEQ ID | Rv3616c | EspA | AEQVGGQSQL |
| | NO. 129 | | | |
| 25 | SEQ ID | Rv3616c | EspA | ASKGTTTKKY |
| | NO. 130 | | | |
| 26 | SEQ ID | Rv3614c | EspD | GWTADPIIGV |
| | NO. 131 | | | |
| 27 | SEQ ID | Rv3614c | EspD | AETDTEQEIALFTVTNPPRTVSVSTLMDGRI |
| | NO. 132 | | | DHVELSARVAWMSESQLASEILVIADLARQK |
| | | | | AQSAQYAFILDRMSQQVDADEHRVALL |
| 28 | SEQ ID | Rv3614c | EspD | AEAEVFATRYS |
| | NO. 133 | | | |
| 29 | SEQ ID | Rv3881c | EspB | AAKNAAQQLVLSADNMREYLAAGAKERQR |
| | NO. 134 | | | LATSLRNAAK |
| 30 | SEQ ID | Rv3881c | EspB | AHFADGWNTFNL |
| | NO. 135 | | | |
| 31 | SEQ ID | Rv3881c | EspB | DQQRQWILHMAKLSAAMAKQAQYVAQLH |
| | NO. 136 | | | VWARREHPTYE |
| 32 | SEQ ID | Rv3881c | EspB | RLYAENPSARDQILPVYAEYQQRSEKVLTEY |
| | NO. 137 | | | NNKAAL |

TABLE 49
List of ORFs known to be immunogenic to CD4 and/or CD8
T cells that were used for epitope discovery for the
“Mixed” HLA class I cassette.

| Gene | Protein name |
|---|---|
| Rv0125 | Mtb32A, pepA |
| Rv0287 | EsxG |
| Rv0288 | EsxH, TB10.4 |
| Rv1047 | |
| Rv1172c | PE12 |
| Rv1195 | PE13 |
| Rv1196 | PPE18, Mtb39a |
| Rv1387 | PPE20 |
| Rv1788 | PE18 |
| Rv1886c | Ag85B, fbpB |
| Rv1908c | katG |
| Rv1926c | mpt63 |
| Rv1980c | mpt64 |
| Rv2031c | hspX |
| Rv2608 | PPE42 |
| Rv2770c | PPE44 |
| Rv2873 | mpt83 |
| Rv2875 | mpt70 |
| Rv3020c | EsxS |
| Rv3024c | trmU |
| Rv3136 | PPE51 |
| Rv3330 | dacB1 |
| Rv3615c | espC |
| Rv3616c | espA |
| Rv3619c | esxV |
| Rv3620c | esxW |
| Rv3804c | Ag85A, fbpA |
| Rv3873 | PPE68 |
| Rv3874 | CFP10, esxB |
| Rv3875 | ESAT-6, esxA |
| Rv3876 | espI |

TABLE 50
Peptides curated from the PopCover-2.0 output selected for the “Mixed” class I
cassette.

| Peptide | SEQ | | | |
|---|---|---|---|---|
| # | ID NO. | gene | protein | amino acid sequence included in cassette |
| 1 | SEQ ID | Rv2031c | hspX | RHPRSLFPEFSELFAAFPSFAGLRPTFDT |
| | NO. | | | RLMR |
| | 138 | | | |
| 2 | SEQ ID | Rv3330 | dacB1 | DTRVATPSGLDGPGMSTSAYDIGLFYR |
| | NO. | | | YAWQNPVF |
| | 139 | | | |
| 3 | SEQ ID | Rv1196 | PPE18, | AARALPLTSLTSAAERGPGQML |
| | NO. | | Mtb39a | |
| | 140 | | | |
| 4 | SEQ ID | Rv3136 | PPE51 | YAMTLPPPVVAANRIQLL |
| | NO. | | | |
| | 141 | | | |
| 5 | SEQ ID | Rv3804c | Ag85A | DFPDSGTHSWE |
| | NO. | | | |
| | 142 | | | |
| 6 | SEQ ID | Rv3804c | Ag85A | TLAIYHPQQFVY |
| | NO. | | | |
| | 143 | | | |
| 7 | SEQ ID | Rv1387 | PPE20 | VEMAAQQHVVIEAYTAAVELMPTQVE |
| | NO. | | | LA |
| | 144 | | | |
| 8 | SEQ ID | Rv1196 | PPE18, | LRVPPRPYVMPHSPAAG |
| | NO. | | | |
| | 145 | | Mtb39a | |
| 9 | SEQ ID | Rv1980c | mpt64 | GGTHPTTTYKAFDWDQAYRKPITY |
| | NO. | | | |
| | 146 | | | |
| 10 | SEQ ID | Rv2873 | mpt83 | SRIDGTHQTL |
| | NO. | | | |
| | 147 | | | |
| 11 | SEQ ID | Rv1387 | PPE20 | PHTSPPPLILKS |
| | NO. | | | |
| | 148 | | | |
| 12 | SEQ ID | Rv2608 | PPE42 | VSSPDVLTTYI |
| | NO. | | | |
| | 149 | | | |
| 13 | SEQ ID | Rv3136 | PPE51 | SQATDPLSLLIETVTQALQALTIPSFIPE |
| | NO. | | | DFTFL |
| | 150 | | | |
| 14 | SEQ ID | Rv1387 | PPE20 | QAAEAFVAAYMPFL |
| | NO. | | | |
| | 151 | | | |
| 15 | SEQ ID | Rv1172c | PE12 | PISPPGFPSLP |
| | NO. | | | |
| | 152 | | | |
| 16 | SEQ ID | Rv1047 | | ATIDVAIPKLR |
| | NO. | | | |
| | 153 | | | |
| 17 | SEQ ID | Rv3330 | dacB1 | GYTDDAGQTFV |
| | NO. | | | |
| | 154 | | | |
| 18 | SEQ ID | Rv1788 | PE18 | SAQAAAIHEMF |
| | NO. | | | |
| | 155 | | | |
| 19 | SEQ ID | Rv3020c | esxS | AAFQGAHARF |
| | NO. | | | |
| | 156 | | | |
| 20 | SEQ ID | Rv1886c | Ag85B | WERNDPTQQI |
| | NO. | | | |
| | 157 | | | |
| 21 | SEQ ID | Rv1908c | katG | VAAWDKVMNL |
| | NO. | | | |
| | 158 | | | |
| 22 | SEQ ID | Rv3024c | trmU | FYVWDFAEKFKEDVINDFVSSY |
| | NO. | | | |
| | 159 | | | |
| 23 | SEQ ID | Rv1387 | PPE20 | AFPPEVHSAML |
| | NO. | | | |
| | 160 | | | |
| 24 | SEQ ID | Rv2608 | PPE42 | AFQYDGVNDFPKYPLNVFATANAIAGI |
| | NO. | | | LFLHSGLIAL |
| | 161 | | | |
| 25 | SEQ ID | Rv2608 | PPE42 | ATIATFEMRYLQSLPAHLRPGLDEL |
| | NO. | | | |
| | 162 | | | |
| 26 | SEQ ID | Rv3873 | PPE68 | IRMWNQAALAMEVYQAETAVNTLF |
| | NO. | | | |
| | 163 | | | |
| 27 | SEQ ID | Rv1926c | mpt63 | FAAPVALAAYPITGKL |
| | NO. | | | |
| | 164 | | | |
| 28 | SEQ ID | Rv2031c | hspX | ATYDKGILTV |
| | NO. | | | |
| | 165 | | | |
| 29 | SEQ ID | Rv0125 | Mtb32A | GFAIPIGQAMA |
| | NO. | | | |
| | 166 | | | |
| 30 | SEQ ID | Rv1886c | Ag85B | ILAAYHPQQFIYAGSLSALL |
| | NO. | | | |
| | 167 | | | |
| 31 | SEQ ID | Rv3616c | espA | VHAASTRQALR |
| | NO. | | | |
| | 168 | | | |
| 32 | SEQ ID | Rv3874 | CFP10 | QELDEISTNI |
| | NO. | | | |
| | 169 | | | |
| 33 | SEQ ID | Rv1926c | mpt64 | GSVTPAVSQFNARTADGINYRV |
| | NO. | | | |
| | 170 | | | |
| 34 | SEQ ID | Rv3024c | trmU | VAELIGDALFVQLHAPLRGVARGQTL |
| | NO. | | | VLY |
| | 171 | | | |
| 35 | SEQ ID | Rv1387 | PPE20 | AAIPAVVPELTPVAAAPPMLAVA |
| | NO. | | | |
| | 172 | | | |
| 36 | SEQ ID | Rv3330 | dacB1 | ELENDNQLLY |
| | NO. | | | |
| | 173 | | | |
| 37 | SEQ ID | Rv3020c | esxS | LLDAHIPQLI |
| | NO. | | | |
| | 174 | | | |
| 38 | SEQ ID | Rv1387 | PPE20 | DYVYPGHAIWWLARGLEFFQDGEQFG |
| | NO. | | | ELLFTNPTGAF |
| | 175 | | | |
| 39 | SEQ ID | Rv3616c | espA | MAVVGGALAYLVVKTLINATQLLKLL |
| | NO. | | | AKL |
| | 176 | | | |
| 40 | SEQ ID | Rv1047 | | KELDEAVEAF |
| | NO. | | | |
| | 177 | | | |
| 41 | SEQ ID | Rv0287 | esxG | SLLDAHIPQLV |
| | NO. | | | |
| | 178 | | | |
| 42 | SEQ ID | Rv1908c | katG | AVADPMGAAFDYAAEV |
| | NO. | | | |
| | 179 | | | |
| 43 | SEQ ID | Rv3616c | espA | TYIPVVGHALS |
| | NO. | | | |
| | 180 | | | |
| 44 | SEQ ID | Rv3615c | espC | SQFNDTLNVY |
| | NO. | | | |
| | 181 | | | |
| 45 | SEQ ID | Rv0125 | Mtb32A | RAVPGRVVAL |
| | NO. | | | |
| | 182 | | | |
| 46 | SEQ ID | Rv2873 | mpt83 | AKLLSSILTYH |
| | NO. | | | |
| | 183 | | | |
| 47 | SEQ ID | Rv3873 | PPE68 | IEKPVAPSVM |
| | NO. | | | |
| | 184 | | | |
| 48 | SEQ ID | Rv1908c | katG | AAAVDIRETFR |
| | NO. | | | |
| | 185 | | | |
| 49 | SEQ ID | Rv2608 | PPE42 | GLPPPWQPAL |
| | NO. | | | |
| | 186 | | | |
| 50 | SEQ ID | Rv3804c | Ag85A | FSRPGLPVEYLQVPSPSMG |
| | NO. | | | |
| | 187 | | | |
| 51 | SEQ ID | Rv2031c | hspX | MVRDGQLTIK |
| | NO. | | | |
| | 188 | | | |
| 52 | SEQ ID | | | AAADEVSTQVA |
| | NO. | | | |
| | 189 | Rv1172c | PE12 | |
| 53 | SEQ ID | Rv2608 | PPE42 | LLPLPNIPLLEYAARFITPVHPGYTATFL |
| | NO. | | | ETPSQFFPFTGLNSLT |
| | 190 | | | |
| 54 | SEQ ID | Rv1908c | katG | SELRALVEVY |
| | NO. | | | |
| | 191 | | | |
| 55 | SEQ ID | Rv2770c | PPE44 | IAVMPPAPGMV |
| | NO. | | | |
| | 192 | | | |
| 56 | SEQ ID | Rv1387 | PPE20 | VRAATTMATY |
| | NO. | | | |
| | 193 | | | |
| 57 | SEQ ID | Rv1047 | | RVLDALTDKL |
| | NO. | | | |
| | 194 | | | |
| 58 | SEQ ID | Rv1172c | PE12 | ILAPQINFFY |
| | NO. | | | |
| | 195 | | | |
| 59 | SEQ ID | Rv1980c | mpt64 | ADTDPLPVVF |
| | NO. | | | |
| | 196 | | | |
| 60 | SEQ ID | Rv3136 | PPE51 | GRASGVLPRY |
| | NO. | | | |
| | 197 | | | |
| 61 | SEQ ID | Rv0288 | esxH, | IMYNYPAMLG |
| | NO. | | TB10.4 | |
| | 198 | | | |
| 62 | SEQ ID | Rv2875 | mpt70 | IDELKTNSSLLTSILTY |
| | NO. | | | |
| | 199 | | | |
| 63 | SEQ ID | Rv1196 | PPE18, | MYAGPGSASL |
| | NO. | | Mtb39a | |
| | 200 | | | |
| 64 | SEQ ID | Rv1886c | Ag85B | YYQSGLSIVM |
| | NO. | | | |
| | 201 | | | |
| 65 | SEQ ID | Rv3620c | esxW | SRFMTDPHAMR |
| | NO. | | | |
| | 202 | | | |
| 66 | SEQ ID | Rv1172c | PE12 | AFHDQFVQALR |
| | NO. | | | |
| | 203 | | | |

TABLE 51
Top optimized solutions for “Mtb-only” peptides using
the basic sum score or the weighted penalized score.

| | | Basic | | N junctions w/ | N total |
|---|---|---|---|---|---|
| | Additional | sum | Penalty | epitope <0.5 | epitopes |
| name | penalty | score | score | rank | w/ <0.5 rank |
| AKG.Mtb-only_p0_sol1 | 0 | 125.03 | 125.03 | 9 | 12 |
| AKG.Mtb-only_p0_sol2 | 0 | 125.84 | 125.84 | 7 | 15 |
| AKG.Mtb-only_p0_sol3 | 0 | 127.97 | 127.97 | 9 | 15 |
| AKG.Mtb-only_p0_sol4 | 0 | 128.19 | 128.19 | 9 | 13 |
| AKG.Mtb-only_p0_sol5 | 0 | 128.49 | 128.49 | 9 | 15 |
| AKG.Mtb-only_p5_sol1 | +5 | 127.97 | 157.97 | 5 | 6 |
| AKG.Mtb-only_p5_sol2 | +5 | 123.34 | 166.34 | 5 | 8 |
| AKG.Mtb-only_p5_sol3 | +5 | 135.42 | 170.41 | 6 | 7 |
| AKG.Mtb-only_p5_sol4 | +5 | 137.98 | 172.98 | 5 | 7 |
| AKG.Mtb-only_p5_sol5 | +5 | 135.22 | 180.22 | 6 | 9 |

TABLE 52
The sequence of peptides for each solution shown in Table 51. Refer
to Table 48 (peptide numbers 1-32) for associated peptide sequences.

| name | Peptide sequence (peptide # from Table 48) |
|---|---|
| AKG.Mtb- | 21, 32, 31, 13, 14, 04, 01, 29, 22, 08, 28, 03, 27, 20, 23, 19, |
| only_p0_{—} | 17, 24, 06, 16, 11, 05, 18, 07, 12, 25, 10, 30, 09, 26, 15, 02 |
| sol1 | |
| AKG.Mtb- | 10, 30, 19, 02, 18, 17, 13, 14, 04, 01, 05, 24, 03, 27, 26, 15, |
| only_p0_{—} | 32, 31, 29, 16, 21, 25, 08, 09, 20, 11, 22, 07, 12, 23, 06, 28 |
| sol2 | |
| AKG.Mtb- | 14, 10, 30, 19, 20, 24, 03, 27, 26, 15, 32, 31, 01, 29, 16, 11, |
| only_p0_{—} | 22, 08, 21, 07, 12, 23, 25, 04, 06, 28, 05, 02, 18, 17, 13, 09 |
| sol3 | |
| AKG.Mtb- | 17, 09, 26, 15, 32, 31, 29, 16, 30, 10, 19, 02, 18, 13, 14, 04, |
| only_p0_{—} | 01, 05, 24, 03, 11, 27, 20, 25, 08, 21, 22, 07, 12, 23, 06, 28 |
| sol4 | |
| AKG.Mtb- | 17, 09, 24, 03, 27, 26, 15, 32, 31, 01, 29, 30, 19, 20, 16, 22, |
| only_p0_{—} | 08, 21, 07, 12, 23, 25, 04, 06, 28, 05, 02, 18, 13, 14, 10, 11 |
| sol5 | |
| AKG.Mtb- | 14, 08, 04, 01, 27, 26, 15, 02, 18, 07, 12, 23, 30, 28, 29, 32, |
| only_p5_{—} | 31, 06, 25, 10, 16, 21, 19, 17, 09, 20, 24, 05, 13, 22, 03, 11 |
| sol1 | |
| AKG.Mtb- | 01, 29, 25, 08, 21, 02, 18, 17, 09, 26, 12, 13, 14, 07, 15, 32, |
| only_p5_{—} | 31, 05, 24, 04, 10, 19, 20, 23, 06, 16, 30, 28, 27, 22, 03, 11 |
| sol2 | |
| AKG.Mtb- | 14, 07, 26, 08, 21, 09, 20, 25, 28, 05, 06, 16, 30, 04, 01, 27, |
| only_p5_{—} | 19, 17, 02, 18, 12, 13, 10, 24, 15, 32, 31, 29, 23, 22, 03, 11 |
| sol3 | |
| AKG.Mtb- | 14, 25, 04, 10, 16, 21, 02, 27, 15, 32, 31, 09, 20, 23, 24, 30, |
| only_p5_{—} | 19, 17, 26, 07, 12, 13, 01, 29, 22, 08, 06, 28, 05, 18, 03, 11 |
| sol4 | |
| AKG.Mtb- | 17, 12, 14, 08, 09, 13, 29, 11, 22, 07, 31, 06, 28, 05, 24, 04, |
| only_p5_{—} | 10, 16, 21, 32, 19, 20, 23, 30, 25, 01, 27, 26, 15, 02, 18, 03 |
| sol5 | |

TABLE 53
Top optimized solutions for “Mixed” peptides using
the basic sum score or the weighted penalized score.

| | | | | N junctions w/ | N total |
|---|---|---|---|---|---|
| | Additional | Basic sum | Penalty | epitope <0.5 | epitopes |
| name | penalty | score | score | rank | w/ <0.5 rank |
| AKG.mixed_p0_sol1 | 0 | 295.27 | 295.27 | 15 | 23 |
| AKG.mixed_p0_sol2 | 0 | 314.41 | 314.41 | 22 | 31 |
| AKG.mixed_p0_sol3 | 0 | 310.38 | 310.38 | 18 | 26 |
| AKG.mixed_p0_sol4 | 0 | 310.57 | 310.57 | 19 | 30 |
| AKG.mixed_p0_sol5 | 0 | 312.77 | 312.77 | 21 | 28 |
| AKG.mixed_p5_sol1 | +5 | 348.40 | 408.40 | 8 | 12 |
| AKG.mixed_p5_sol2 | +5 | 319.53 | 414.53 | 17 | 19 |
| AKG.mixed_p5_sol3 | +5 | 341.31 | 416.31 | 13 | 15 |
| AKG.mixed_p5_sol4 | +5 | 331.08 | 421.08 | 15 | 18 |
| AKG.mixed_p5_sol5 | +5 | 344.39 | 424.39 | 12 | 16 |

TABLE 54
The sequence of peptides for each solution shown in Table
53. Refer to Table 50 for associated peptide sequences.

| name | Peptide sequence (peptide # from Table 50) |
|---|---|
| AKG.mixed_{—} | 22, 11, 20, 13, 10, 34, 26, 21, 43, 39, 60, 49, 01, 41, 66, 35, 28, 64, 02, 09, 47, 30, 36, 61, 31, |
| p0_sol1 | 07, 51, 46, 33, 06, 65, 48, 54, 53, 03, 38, 05, 29, 17, 32, 14, 12, 52, 40, 62, 55, 45, 50, 19, |
| | 23, 58, 18, 24, 59, 16, 44, 56, 15, 63, 57, 25, 04, 37, 42, 08, 27 |
| AKG.mixed_{—} | 22, 57, 25, 08, 27, 32, 60, 66, 14, 49, 01, 38, 37, 42, 45, 16, 44, 05, 23, 47, 02, 52, 48, 33, 43, |
| p0_sol2 | 39, 15, 04, 31, 28, 63, 07, 41, 58, 30, 53, 51, 46, 35, 13, 29, 17, 65, 21, 64, 03, 26, 34, 06, |
| | 55, 19, 12, 09, 61, 50, 54, 62, 11, 20, 56, 36, 10, 18, 24, 59, 40 |
| AKG.mixed_{—} | 22, 42, 63, 32, 56, 12, 04, 41, 58, 06, 02, 09, 61, 59, 28, 51, 38, 34, 21, 43, 55, 50, 44, 36, 10, |
| p0_sol3 | 17, 19, 23, 48, 39, 25, 08, 57, 49, 01, 35, 13, 31, 07, 52, 40, 62, 46, 33, 29, 64, 18, 05, 45, |
| | 16, 54, 15, 20, 47, 30, 53, 03, 26, 24, 65, 27, 60, 37, 11, 66, 14 |
| AKG.mixed_{—} | 22, 33, 43, 26, 21, 23, 13, 48, 39, 60, 49, 01, 41, 14, 59, 27, 25, 04, 32, 12, 09, 61, 50, 47, 30, |
| p0_sol4 | 34, 07, 17, 65, 56, 18, 24, 52, 40, 53, 03, 38, 64, 02, 62, 46, 54, 15, 05, 31, 58, 06, 55, 28, |
| | 63, 19, 29, 36, 08, 57, 66, 35, 10, 37, 42, 45, 44, 11, 20, 51, 16 |
| AKG.mixed_{—} | 08, 16, 44, 27, 25, 28, 10, 34, 26, 21, 14, 49, 01, 24, 57, 66, 46, 35, 11, 29, 63, 65, 38, 06, 47, |
| p0_sol5 | 30, 36, 31, 07, 13, 60, 64, 39, 23, 12, 09, 61, 56, 53, 02, 18, 62, 15, 04, 48, 40, 05, 37, 59, |
| | 20, 03, 22, 42, 45, 51, 17, 19, 33, 43, 41, 52, 32, 58, 55, 50, 54 |
| AKG.mixed_{—} | 22, 42, 08, 35, 47, 05, 45, 41, 57, 59, 20, 56, 12, 52, 62, 27, 48, 39, 25, 16, 09, 61, 64, 03, 34, |
| p5_sol1 | 07, 02, 66, 46, 54, 15, 04, 65, 29, 63, 28, 17, 32, 14, 58, 30, 36, 38, 06, 55, 50, 19, 60, 49, |
| | 01, 10, 21, 43, 26, 13, 31, 44, 53, 37, 18, 24, 11, 51, 33, 23, 40 |
| AKG.mixed_{—} | 61, 56, 15, 04, 50, 28, 03, 22, 10, 17, 19, 23, 07, 51, 13, 38, 64, 11, 54, 34, 48, 39, 49, 01, 29, |
| p5_sol2 | 41, 18, 58, 06, 55, 57, 66, 25, 45, 31, 44, 36, 46, 33, 65, 21, 43, 26, 24, 59, 20, 37, 42, 08, |
| | 16, 09, 47, 30, 05, 35, 63, 32, 60, 14, 12, 52, 40, 53, 02, 62, 27 |
| AKG.mixed_{—} | 22, 63, 07, 17, 19, 13, 42, 08, 23, 16, 44, 15, 10, 26, 18, 62, 48, 39, 49, 01, 24, 36, 21, 43, 51, |
| p5_sol3 | 40, 56, 12, 20, 03, 38, 11, 60, 29, 50, 32, 14, 52, 31, 47, 02, 66, 35, 28, 64, 41, 09, 57, 25, |
| | 04, 61, 58, 06, 65, 37, 59, 27, 34, 55, 45, 46, 33, 30, 53, 05, 54 |
| AKG.mixed_{—} | 22, 64, 07, 51, 02, 33, 14, 58, 30, 53, 03, 20, 47, 12, 09, 57, 25, 08, 23, 16, 27, 59, 04, 65, 31, |
| p5_sol4 | 28, 43, 48, 39, 17, 19, 54, 34, 26, 24, 61, 56, 15, 36, 46, 13, 29, 42, 05, 45, 63, 32, 60, 49, |
| | 01, 38, 06, 62, 11, 66, 35, 55, 50, 44, 37, 18, 21, 10, 41, 52, 40 |
| AKG.mixed_{—} | 22, 40, 36, 61, 64, 03, 26, 48, 44, 46, 33, 30, 11, 65, 51, 29, 21, 43, 55, 45, 27, 25, 60, 49, 01, |
| p5_sol5 | 13, 19, 66, 35, 42, 08, 17, 32, 56, 15, 05, 24, 57, 58, 06, 28, 63, 10, 38, 31, 07, 37, 14, 52, |
| | 02, 62, 16, 59, 04, 50, 54, 53, 34, 39, 23, 20, 47, 41, 18, 12, 09 |

SEQ ID NOs associated with proteins listed in Table 47.

SEQ ID NO. 45
Mtb ORF Rv1980c/MPT64_signal sequence (aa 1-23) removed.
APKTYCEELKGTDTGQACQIQMSDPAYNINISLPSYYPDQKSLENYIAQTRDKFLSAATSSTPR
EAPYELNITSATYQSAIPPRGTQAVVLKVYQNAGGTHPTTTYKAFDWDQAYRKPITYDTLWQAD
TDPLPVVFPIVQGELSKQTGQQVSIAPNAGLDPVNYQNFAVTNDGVIFFENPGELLPEAAGPTQ
VLVPRSAIDSMLA
SEQ ID NO. 46
Mtb ORF Rv3873/PPE68_AA 155-219 (areas with homology to BCG
removed).
QAETAVNTLFEKLEPMASILDPGASQSTINPIFGMPSPGSSTPVGQLPPAATQTLGQLGEMSGP
M
SEQ ID NO. 47
Mtb ORF Rv3873/PPE68_AA 242-305 (areas with homology to BCG
removed).
GNPADEEAAQMGLLGTSPLSNHPLAGGSGPSAGAGLLRAESLPGAGGSLTRTPLMSQLIEKPVA
SEQ ID NO. 48
Mtb ORF Rv3876/EspI.
MAADYDKLFRPHEGMEAPDDMAAQPFEDPSASFPPAPASANLPKPNGQTPPPTSDDLSERFVSA
PPPPPPPPPPPPPTPMPIAAGEPPSPEPAASKPPTPPMPIAGPEPAPPKPPTPPMPIAGPEPAP
PKPPTPPMPIAGPAPTPTESQLAPPRPPTPQTPTGAPQQPESPAPHVPSHGPHQPRRTAPAPPW
AKMPIGEPPPAPSRPSASPAEPPTRPAPQHSRRARRGHRYRTDTERNVGKVATGPSIQARLRAE
EASGAQLAPGTEPSPAPLGQPRSYLAPPTRPAPTEPPPSPSPQRNSGRRAERRVHPDLAAQHAA
AQPDSITAATTGGRRRKRAAPDLDATQKSLRPAAKGPKVKKVKPQKPKATKPPKVVSQRGWRHW
VHALTRINLGLSPDEKYELDLHARVRRNPRGSYQIAVVGLKGGAGKTTLTAALGSTLAQVRADR
ILALDADPGAGNLADRVGRQSGATIADVLAEKELSHYNDIRAHTSVNAVNLEVLPAPEYSSAQR
ALSDADWHFIADPASRFYNLVLADCGAGFFDPLTRGVLSTVSGVVVVASVSIDGAQQASVALDW
LRNNGYQDLASRACVVINHIMPGEPNVAVKDLVRHFEQQVQPGRVVVMPWDRHIAAGTEISLDL
LDPIYKRKVLELAAALSDDFERAGRR
SEQ ID NO. 49
Mtb ORF Rv3615c/EspC.
MTENLTVQPERLGVLASHHDNAAVDASSGVEAAAGLGESVAITHGPYCSQFNDTLNVYLTAHNA
LGSSLHTAGVDLAKSLRIAAKIYSEADEAWRKAIDGLFT
SEQ ID NO. 50
Mtb ORF Rv3616c/EspA.
MSRAFIIDPTISAIDGLYDLLGIGIPNQGGILYSSLEYFEKALEELAAAFPGDGWLGSAADKYA
GKNRNHVNFFQELADLDRQLISLIHDQANAVQTTRDILEGAKKGLEFVRPVAVDLTYIPVVGHA
LSAAFQAPFCAGAMAVVGGALAYLVVKTLINATQLLKLLAKLAELVAAAIADIISDVADIIKGT
LGEVWEFITNALNGLKELWDKLTGWVTGLFSRGWSNLESFFAGVPGLTGATSGLSQVTGLFGAA
GLSASSGLAHADSLASSASLPALAGIGGGSGFGGLPSLAQVHAASTRQALRPRADGPVGAAAEQ
VGGQSQLVSAQGSQGMGGPVGMGGMHPSSGASKGTTTKKYSEGAAAGTEDAERAPVEADAGGGQ
KVLVRNVV
SEQ ID NO. 51
Mtb ORF Rv2875/MPT70_signal sequence (aa 1-30) removed.
GDLVGPGCAEYAAANPTGPASVQGMSQDPVAVAASNNPELTTLTAALSGQLNPQVNLVDTLNSG
QYTVFAPTNAAFSKLPASTIDELKTNSSLLTSILTYHVVAGQTSPANVVGTRQTLQGASVTVTG
QGNSLKVGNADVVCGGVSTANATVYMIDSVLMPPA
SEQ ID NO. 52
Mtb ORF Rv2873/MPT83_signal sequence (aa 1-30) removed.
VSQDTSPKPATSPAAPVTTAAMADPAADLIGRGCAQYAAQNPTGPGSVAGMAQDPVATAASNNP
MLSTLTSALSGKLNPDVNLVDTLNGGEYTVFAPTNAAFDKLPAATIDQLKTDAKLLSSILTYHV
IAGQASPSRIDGTHQTLQGADLTVIGARDDLMVNNAGLVCGGVHTANATVYMIDTVLMPPAQ
SEQ ID NO. 53
Mtb ORF Rv3614c/EspD.
VDLPGNDFDSNDFDAVDLWGADGAEGWTADPIIGVGSAATPDTGPDLDNAHGQAETDTEQEIAL
FTVTNPPRTVSVSTLMDGRIDHVELSARVAWMSESQLASEILVIADLARQKAQSAQYAFILDRM
SQQVDADEHRVALLRKTVGETWGLPSPEEAAAAEAEVFATRYSDDCPAPDDESDPW
SEQ ID NO. 54
Mtb ORF Rv3881c/EspB.
MTQSQTVTVDQQEILNRANEVEAPMADPPTDVPITPCELTAAKNAAQQLVLSADNMREYLAAGA
KERQRLATSLRNAAKAYGEVDEEAATALDNDGEGTVQAESAGAVGGDSSAELTDTPRVATAGEP
NEMDLKEAARKLETGDQGASLAHFADGWNTFNLTLQGDVKRFRGFDNWEGDAATACEASLDQQR
QWILHMAKLSAAMAKQAQYVAQLHVWARREHPTYEDIVGLERLYAENPSARDQILPVYAEYQQR
SEKVLTEYNNKAALEPVNPPKPPPAIKIDPPPPPQEQGLIPGFLMPPSDGSGVTPGTGMPAAPM
VPPTGSPGGGLPADTAAQLTSAGREAAALSGDVAVKAASLGGGGGGGVPSAPLGSAIGGAESVR
PAGAGDIAGLGQGRAGGGAALGGGGMGMPMGAAHQGQGGAKSKGSQQEDEALYTEDRAWTEAVI
GNRRRQDSKESK

SEQ ID NOs associated with Table 49.

SEQ ID NO. 55
Mycobacterium tuberculosis H37Rv|Rv0125|pepA|Mtb32A.
MSNSRRRSLRWSWLLSVLAAVGLGLATAPAQAAPPALSQDRFADFPALPLDPSAMVAQVGPQVV
NINTKLGYNNAVGAGTGIVIDPNGVVLTNNHVIAGATDINAFSVGSGQTYGVDVVGYDRTQDVA
VLQLRGAGGLPSAAIGGGVAVGEPVVAMGNSGGQGGTPRAVPGRVVALGQTVQASDSLTGAEET
LNGLIQFDAAIQPGDSGGPVVNGLGQVVGMNTAASDNFQLSQGGQGFAIPIGQAMAIAGQIRSG
GGSPTVHIGPTAFLGLGVVDNNGNGARVQRVVGSAPAASLGISTGDVITAVDGAPINSATAMAD
ALNGHHPGDVISVTWQTKSGGTRTGNVTLAEGPPA
SEQ ID NO. 56
Mycobacterium tuberculosis H37Rv|Rv0287|esxG.
MSLLDAHIPQLVASQSAFAAKAGLMRHTIGQAEQAAMSAQAFHQGESSAAFQAAHARFVAAAAK
VNTLLDVAQANLGEAAGTYVAADAAAASTYTGF
SEQ ID NO. 57
Mycobacterium tuberculosis H37Rv|Rv0288|esxH|TB10.4.
MSQIMYNYPAMLGHAGDMAGYAGTLQSLGAEIAVEQAALQSAWQGDTGITYQAWQAQWNQAMED
LVRAYHAMSSTHEANTMAMMARDTAEAAKWGG
SEQ ID NO. 58
Mycobacterium tuberculosis H37Rv|Rv1047|Rv1047.
MTSSHLIDAEQLLADQLAQASPDLLRGLLSTFIAALMGAEADALCGAGYRERSDERSNQRNGYR
HRDFDTRAATIDVAIPKLRQGSYFPDWLLQRRKRAERALTSVVATCYLLGVSTRRMERLVETLG
VTKLSKSQVSIMAKELDEAVEAFRTRPLDAGPYTFLAADALVLKVREAGRVVGVHTLIATGVNA
EGYREILGIQVTSAEDGAGWLAFFRDLVARGLSGVALVTSDAHAGLVAAIGATLPAAAWQRCRT
HYAANLMAATPKPSWPWVRTLLHSIYDQPDAESVVAQYDRVLDALTDKLPAVAEHLDTARTDLL
AFTAFPKQIWRQIWSNNPQERLNREVRRRTDVVGIFPDRASIIRLVGAVLAEQHDEWIEGRRYL
GLEVLTRARAALTSTEEPAKQQTTNTPALTT
SEQ ID NO. 59
Mycobacterium tuberculosis H37Rv|Rv1172c|PE12.
MSFVFAAPEALAAAAADMAGIGSTLNAANVVAAVPTTGVLAAAADEVSTQVAALLSAHAQGYQQ
LSRQMMTAFHDQFVQALRASADAYATAEASAAQTMVNAVNAPARALLGHPLISADASTGGGSNA
LSRVQSMFLGTGGSSALGGSAAANAAASGALQLQPTGGASGLSAVGALLPRAGAAAAAALPALA
AESIGNAIKNLYNAVEPWVQYGENLTAWAVGWLPYIGILAPQINFFYYLGEPIVQAVLENAIDE
VDGTVTFSQALTNIETATAASINQFINTEINWIRGFLPPLPPISPPGFPSLP
SEQ ID NO. 60
Mycobacterium tuberculosis H37Rv|Rv1195|PE13.
VSFVMAYPEMLAAAADTLQSIGATTVASNAAAAAPTTGVVPPAADEVSALTAAHFAAHAAMYQS
VSARAAAIHDQFVATLASSASSYAATEVANAAAAS
SEQ ID NO. 61
Mycobacterium tuberculosis H37Rv|Rv1196|PPE18|Mtb39a.
MVDFGALPPEINSARMYAGPGSASLVAAAQMWDSVASDLFSAASAFQSVVWGLTVGSWIGSSAG
LMVAAASPYVAWMSVTAGQAELTAAQVRVAAAAYETAYGLTVPPPVIAENRAELMILIATNLLG
QNTPAIAVNEAEYGEMWAQDAAAMFGYAAATATATATLLPFEEAPEMTSAGGLLEQAAAVEEAS
DTAAANQLMNNVPQALQQLAQPTQGTTPSSKLGGLWKTVSPHRSPISNMVSMANNHMSMTNSGV
SMTNTLSSMLKGFAPAAAAQAVQTAAQNGVRAMSSLGSSLGSSGLGGGVAANLGRAASVGSLSV
PQAWAAANQAVTPAARALPLTSLTSAAERGPGQMLGGLPVGQMGARAGGGLSGVLRVPPRPYVM
PHSPAAG
SEQ ID NO. 62
Mycobacterium tuberculosis H37Rv|Rv1387|PPE20.
MTEPWIAFPPEVHSAMLNYGAGVGPMLISATQNGELSAQYAEAASEVEELLGVVASEGWQGQAA
EAFVAAYMPFLAWLIQASADCVEMAAQQHVVIEAYTAAVELMPTQVELAANQIKLAVLVATNFF
GINTIPIAINEAEYVEMWVRAATTMATYSTVSRSALSAMPHTSPPPLILKSDELLPDTGEDSDE
DGHNHGGHSHGGHARMIDNFFAEILRGVSAGRIVWDPVNGTLNGLDYDDYVYPGHAIWWLARGL
EFFQDGEQFGELLFTNPTGAFQFLLYVVVVDLPTHIAQIATWLGQYPQLLSAALTGVIAHLGAI
TGLAGLSGLSAIPSAAIPAVVPELTPVAAAPPMLAVAGVGPAVAAPGMLPASAPAPAAAAGATA
AGPTPPATGFGGFPPYLVGGGGPGIGFGSGQSAHAKAAASDSAAAESAAQASARAQARAARRGR
SAAKARGHRDEFVTMDMGFDAAAPAPEHQPGARASDCGAGPIGFAGTVRKEAVVKAAGLTTLAG
DDFGGGPTMPMMPGTWTHDQGVFDEHR
SEQ ID NO. 63
Mycobacterium tuberculosis H37Rv|Rv1788|PE18.
MSFVTTQPEALAAAAGSLQGIGSALNAQNAAAATPTTGVVPAAADEVSALTAAQFAAHAQIYQA
VSAQAAAIHEMFVNTLQMSSGSYAATEAANAAAAG
SEQ ID NO. 64
Mycobacterium tuberculosis H37Rv|Rv1886c|fbpB|Ag85B.
MTDVSRKIRAWGRRLMIGTAAAVVLPGLVGLAGGAATAGAFSRPGLPVEYLQVPSPSMGRDIKV
QFQSGGNNSPAVYLLDGLRAQDDYNGWDINTPAFEWYYQSGLSIVMPVGGQSSFYSDWYSPACG
KAGCQTYKWETFLTSELPQWLSANRAVKPTGSAAIGLSMAGSSAMILAAYHPQQFIYAGSLSAL
LDPSQGMGPSLIGLAMGDAGGYKAADMWGPSSDPAWERNDPTQQIPKLVANNTRLWVYCGNGTP
NELGGANIPAEFLENFVRSSNLKFQDAYNAAGGHNAVENFPPNGTHSWEYWGAQLNAMKGDLQS
SLGAG
SEQ ID NO. 65
Mycobacterium tuberculosis H37Rv|Rv1908c|katG.
VPEQHPPITETTTGAASNGCPVVGHMKYPVEGGGNQDWWPNRLNLKVLHQNPAVADPMGAAFDY
AAEVATIDVDALTRDIEEVMTTSQPWWPADYGHYGPLFIRMAWHAAGTYRIHDGRGGAGGGMQR
FAPLNSWPDNASLDKARRLLWPVKKKYGKKLSWADLIVFAGNCALESMGFKTFGFGFGRVDQWE
PDEVYWGKEATWLGDERYSGKRDLENPLAAVQMGLIYVNPEGPNGNPDPMAAAVDIRETFRRMA
MNDVETAALIVGGHTFGKTHGAGPADLVGPEPEAAPLEQMGLGWKSSYGTGTGKDAITSGIEVV
WTNTPTKWDNSFLEILYGYEWELTKSPAGAWQYTAKDGAGAGTIPDPFGGPGRSPTMLATDLSL
RVDPIYERITRRWLEHPEELADEFAKAWYKLIHRDMGPVARYLGPLVPKQTLLWQDPVPAVSHD
LVGEAEIASLKSQIRASGLTVSQLVSTAWAAASSFRGSDKRGGANGGRIRLQPQVGWEVNDPDG
DLRKVIRTLEEIQESFNSAAPGNIKVSFADLVVLGGCAAIEKAAKAAGHNITVPFTPGRTDASQ
EQTDVESFAVLEPKADGFRNYLGKGNPLPAEYMLLDKANLLTLSAPEMTVLVGGLRVLGANYKR
LPLGVFTEASESLINDFFVNLLDMGITWEPSPADDGTYQGKDGSGKVKWTGSRVDLVFGSNSEL
RALVEVYGADDAQPKFVQDEVAAWDKVMNLDRFDVR
SEQ ID NO. 66
Mycobacterium tuberculosis H37Rv|Rv1926c|mpt63.
MKLTTMIKTAVAVVAMAAIATFAAPVALAAYPITGKLGSELTMTDTVGQVVLGWKVSDLKSSTA
VIPGYPVAGQVWEATATVNAIRGSVTPAVSQFNARTADGINYRVLWQAAGPDTISGATIPQGEQ
STGKIYFDVTGPSPTIVAMNNGMEDLLIWEP
SEQ ID NO. 67
Mycobacterium tuberculosis H37Rv|Rv1980c|mpt64.
VRIKIFMLVTAVVLLCCSGVATAAPKTYCEELKGTDTGQACQIQMSDPAYNINISLPSYYPDQK
SLENYIAQTRDKFLSAATSSTPREAPYELNITSATYQSAIPPRGTQAVVLKVYQNAGGTHPTTT
YKAFDWDQAYRKPITYDTLWQADTDPLPVVFPIVQGELSKQTGQQVSIAPNAGLDPVNYQNFAV
TNDGVIFFFNPGELLPEAAGPTQVLVPRSAIDSMLA
SEQ ID NO. 68
Mycobacterium tuberculosis H37Rv|Rv2031c|hspX.
MATTLPVQRHPRSLFPEFSELFAAFPSFAGLRPTFDTRLMRLEDEMKEGRYEVRAELPGVDPDK
DVDIMVRDGQLTIKAERTEQKDFDGRSEFAYGSFVRTVSLPVGADEDDIKATYDKGILTVSVAV
SEGKPTEKHIQIRSTN
SEQ ID NO. 69
Mycobacterium tuberculosis H37Rv|Rv2608|PPE42.
MNFAVLPPEVNSARIFAGAGLGPMLAAASAWDGLAEELHAAAGSFASVTTGLAGDAWHGPASLA
MTRAASPYVGWLNTAAGQAAQAAGQARLAASAFEATLAATVSPAMVAANRTRLASLVAANLLGQ
NAPAIAAAEAEYEQIWAQDVAAMFGYHSAASAVATQLAPIQEGLQQQLQNVLAQLASGNLGSGN
VGVGNIGNDNIGNANIGFGNRGDANIGIGNIGDRNLGIGNTGNWNIGIGITGNGQIGFGKPANP
DVLVVGNGGPGVTALVMGGTDSLLPLPNIPLLEYAARFITPVHPGYTATFLETPSQFFPFTGLN
SLTYDVSVAQGVTNLHTAIMAQLAAGNEVVVFGTSQSATIATFEMRYLQSLPAHLRPGLDELSF
TLTGNPNRPDGGILTRFGFSIPQLGFTLSGATPADAYPTVDYAFQYDGVNDFPKYPLNVFATAN
AIAGILFLHSGLIALPPDLASGVVQPVSSPDVLTTYILLPSQDLPLLVPLRAIPLLGNPLADLI
QPDLRVLVELGYDRTAHQDVPSPFGLFPDVDWAEVAADLQQGAVQGVNDALSGLGLPPPWQPAL
PRLF
SEQ ID NO. 70
Mycobacterium tuberculosis H37Rv|Rv2770c|PPE44.
VDFGALPPEVNSARMYGGAGAADLLAAAAAWNGIAVEVSTAASSVGSVITRLSTEHWMGPASLS
MAAAVQPYLVWLTCTAESSALAAAQAMASAAAFETAFALTVPPAEVVANRALLAELTATNILGQ
NVSAIAATEARYGEMWAQDASAMYGYAAASAVAARLNPLTRPSHITNPAGLAHQAAAVGQAGAS
AFARQVGLSHLISDVADAVLSFASPVMSAADTGLEAVRQFLNLDVPLFVESAFHGLGGVADFAT
AAIGNMTLLADAMGTVGGAAPGGGAAAAVAHAVAPAGVGGTALTADLGNASVVGRLSVPASWST
AAPATAAGAALDGTGWAVPEEDGPIAVMPPAPGMVVAANSVGADSGPRYGVKPIVMPKHGLF
SEQ ID NO. 71
Mycobacterium tuberculosis H37Rv|Rv2873|mpt83.
MINVQAKPAAAASLAAIAIAFLAGCSSTKPVSQDTSPKPATSPAAPVTTAAMADPAADLIGRGC
AQYAAQNPTGPGSVAGMAQDPVATAASNNPMLSTLTSALSGKLNPDVNLVDTLNGGEYTVFAPT
NAAFDKLPAATIDQLKTDAKLLSSILTYHVIAGQASPSRIDGTHQTLQGADLTVIGARDDLMVN
NAGLVCGGVHTANATVYMIDTVLMPPAQ
SEQ ID NO. 72
Mycobacterium tuberculosis H37Rv|Rv2875|mpt70.
MKVKNTIAATSFAAAGLAALAVAVSPPAAAGDLVGPGCAEYAAANPTGPASVQGMSQDPVAVAA
SNNPELTTLTAALSGQLNPQVNLVDTLNSGQYTVFAPTNAAFSKLPASTIDELKTNSSLLTSIL
TYHVVAGQTSPANVVGTRQTLQGASVTVTGQGNSLKVGNADVVCGGVSTANATVYMIDSVLMPP
A
SEQ ID 73
Mycobacterium tuberculosis H37Rv|Rv3020c|esxS.
MSLLDAHIPQLIASHTAFAAKAGLMRHTIGQAEQQAMSAQAFHQGESAAAFQGAHARFVAAAAK
VNTLLDIAQANLGEAAGTYVAADAAAASSYTGF
SEQ ID NO. 74
Mycobacterium tuberculosis H37Rv|Rv3024c|trmU.
MKVLAAMSGGVDSSVAAARMVDAGHEVVGVHMALSTAPGTLRTGSRGCCSKEDAADARRVADVL
GIPFYVWDFAEKFKEDVINDFVSSYARGETPNPCVRCNQQIKFAALSARAVALGFDTVATGHYA
RLSGGRLRRAVDRDKDQSYVLAVLTAQQLRHAAFPIGDTPKRQIRAEAARRGLAVANKPDSHDI
CFIPSGNTKAFLGERIGVRRGVVVDADGVVLASHDGVHGFTIGQRRGLGIAGPGPNGRPRYVTA
IDADTATVHVGDVTDLDVQTLTGRAPVFTAGAAPSGPVDCVVQVRAHGETVSAVAELIGDALFV
QLHAPLRGVARGQTLVLYRPDPAGDEVLGSATIAGASGLSTGGNPGA
SEQ ID NO. 75
Mycobacterium tuberculosis H37Rv|Rv3136|PPE51.
MDFALLPPEVNSARMYTGPGAGSLLAAAGGWDSLAAELATTAEAYGSVLSGLAALHWRGPAAES
MAVTAAPYIGWLYTTAEKTQQTAIQARAAALAFEQAYAMTLPPPVVAANRIQLLALIATNFFGQ
NTAAIAATEAQYAEMWAQDAAAMYGYATASAAAALLTPFSPPRQTTNPAGLTAQAAAVSQATDP
LSLLIETVTQALQALTIPSFIPEDFTFLDAIFAGYATVGVTQDVESFVAGTIGAESNLGLLNVG
DENPAEVTPGDFGIGELVSATSPGGGVSASGAGGAASVGNTVLASVGRANSIGQLSVPPSWAAP
STRPVSALSPAGLTTLPGTDVAEHGMPGVPGVPVAAGRASGVLPRYGVRLTVMAHPPAAG
SEQ ID NO. 76
Mycobacterium tuberculosis H37Rv|Rv3330|dacB1.
MAFLRSVSCLAAAVFAVGTGIGLPTAAGEPNAAPAACPYKVSTPPAVDSSEVPAAGEPPLPLVV
PPTPVGGNALGGCGIITAPGSAPAPGDVSAEAWLVADLDSGAVIAARDPHGRHRPASVIKVLVA
MASINTLTLNKSVAGTADDAAVEGTKVGVNTGGTYTVNQLLHGLLMHSGNDAAYALARQLGGMP
AALEKINLLAAKLGGRDTRVATPSGLDGPGMSTSAYDIGLFYRYAWQNPVFADIVATRTFDFPG
HGDHPGYELENDNQLLYNYPGALGGKTGYTDDAGQTFVGAANRDGRRLMTVLLHGTRQPIPPWE
QAAHLLDYGFNTPAGTQIGTLIEPDPSLMSTDRNPADRQRVDPQAAARISAADALPVRVGVAVI
GALIVFGLIMVARAMNRRPQH
SEQ ID NO. 77
Mycobacterium tuberculosis H37Rv|Rv3615c|espC.
MTENLTVQPERLGVLASHHDNAAVDASSGVEAAAGLGESVAITHGPYCSQFNDTLNVYLTAHNA
LGSSLHTAGVDLAKSLRIAAKIYSEADEAWRKAIDGLFT
SEQ ID NO. 78
Mycobacterium tuberculosis H37Rv|Rv3616c|espA.
MSRAFIIDPTISAIDGLYDLLGIGIPNQGGILYSSLEYFEKALEELAAAFPGDGWLGSAADKYA
GKNRNHVNFFQELADLDRQLISLIHDQANAVQTTRDILEGAKKGLEFVRPVAVDLTYIPVVGHA
LSAAFQAPFCAGAMAVVGGALAYLVVKTLINATQLLKLLAKLAELVAAAIADIISDVADIIKGT
LGEVWEFITNALNGLKELWDKLTGWVTGLFSRGWSNLESFFAGVPGLTGATSGLSQVTGLFGAA
GLSASSGLAHADSLASSASLPALAGIGGGSGFGGLPSLAQVHAASTRQALRPRADGPVGAAAEQ
VGGQSQLVSAQGSQGMGGPVGMGGMHPSSGASKGTTTKKYSEGAAAGTEDAERAPVEADAGGGQ
KVLVRNVV
SEQ ID NO. 79
Mycobacterium tuberculosis H37Rv|Rv3619c|esxV.
MTINYQFGDVDAHGAMIRAQAGSLEAEHQAIISDVLTASDFWGGAGSAACQGFITQLGRNFQVI
YEQANAHGQKVQAAGNNMAQTDSAVGSSWA
SEQ ID NO. 80
Mycobacterium tuberculosis H37Rv|Rv3620c|esxW.
MTSRFMTDPHAMRDMAGRFEVHAQTVEDEARRMWASAQNISGAGWSGMAEATSLDTMTQMNQAF
RNIVNMLHGVRDGLVRDANNYEQQEQASQQILSS
SEQ ID NO. 81
Mycobacterium tuberculosis H37Rv|Rv3804c|fbpA|Ag85A.
MQLVDRVRGAVTGMSRRLVVGAVGAALVSGLVGAVGGTATAGAFSRPGLPVEYLQVPSPSMGRD
IKVQFQSGGANSPALYLLDGLRAQDDFSGWDINTPAFEWYDQSGLSVVMPVGGQSSFYSDWYQP
ACGKAGCQTYKWETFLTSELPGWLQANRHVKPTGSAVVGLSMAASSALTLAIYHPQQFVYAGAM
SGLLDPSQAMGPTLIGLAMGDAGGYKASDMWGPKEDPAWQRNDPLLNVGKLIANNTRVWVYCGN
GKPSDLGGNNLPAKFLEGFVRTSNIKFQDAYNAGGGHNGVFDFPDSGTHSWEYWGAQLNAMKPD
LQRALGATPNTGPAPQGA
SEQ ID NO. 82
Mycobacterium tuberculosis H37Rv|Rv3873|PPE68.
MLWHAMPPELNTARLMAGAGPAPMLAAAAGWQTLSAALDAQAVELTARLNSLGEAWTGGGSDKA
LAAATPMVVWLQTASTQAKTRAMQATAQAAAYTQAMATTPSLPEIAANHITQAVLTATNFFGIN
TIPIALTEMDYFIRMWNQAALAMEVYQAETAVNTLFEKLEPMASILDPGASQSTTNPIFGMPSP
GSSTPVGQLPPAATQTLGQLGEMSGPMQQLTQPLQQVTSLFSQVGGTGGGNPADEEAAQMGLLG
TSPLSNHPLAGGSGPSAGAGLLRAESLPGAGGSLTRTPLMSQLIEKPVAPSVMPAAAAGSSATG
GAAPVGAGAMGQGAQSGGSTRPGLVAPAPLAQEREEDDEDDWDEEDDW
SEQ ID NO. 83
Mycobacterium tuberculosis H37Rv|Rv3874|esxB|CFP10.
MAEMKTDAATLAQEAGNFERISGDLKTQIDQVESTAGSLQGQWRGAAGTAAQAAVVRFQEAANK
QKQELDEISTNIRQAGVQYSRADEEQQQALSSQMGF
SEQ ID NO. 84
Mycobacterium tuberculosis H37Rv|Rv3875|esxA|ESAT-6.
MTEQQWNFAGIEAAASAIQGNVTSIHSLLDEGKQSLTKLAAAWGGSGSEAYQGVQQKWDATATE
LNNALQNLARTISEAGQAMASTEGNVTGMFA
SEQ ID NO. 85
Mycobacterium tuberculosis H37Rv|Rv3876|espI.
MAADYDKLFRPHEGMEAPDDMAAQPFFDPSASFPPAPASANLPKPNGQTPPPTSDDLSERFVSA
PPPPPPPPPPPPPTPMPIAAGEPPSPEPAASKPPTPPMPIAGPEPAPPKPPTPPMPIAGPEPAP
PKPPTPPMPIAGPAPTPTESQLAPPRPPTPQTPTGAPQQPESPAPHVPSHGPHQPRRTAPAPPW
AKMPIGEPPPAPSRPSASPAEPPTRPAPQHSRRARRGHRYRTDTERNVGKVATGPSIQARLRAE
EASGAQLAPGTEPSPAPLGQPRSYLAPPTRPAPTEPPPSPSPQRNSGRRAERRVHPDLAAQHAA
AQPDSITAATTGGRRRKRAAPDLDATQKSLRPAAKGPKVKKVKPQKPKATKPPKVVSQRGWRHW
VHALTRINLGLSPDEKYELDLHARVRRNPRGSYQIAVVGLKGGAGKTTLTAALGSTLAQVRADR
ILALDADPGAGNLADRVGRQSGATIADVLAEKELSHYNDIRAHTSVNAVNLEVLPAPEYSSAQR
ALSDADWHFIADPASRFYNLVLADCGAGFFDPLTRGVLSTVSGVVVVASVSIDGAQQASVALDW
LRNNGYQDLASRACVVINHIMPGEPNVAVKDLVRHFEQQVQPGRVVVMPWDRHIAAGTEISLDL
LDPIYKRKVLELAAALSDDFERAGRR

Amino acid sequences for optimized solutions shown in Tables 51 and 52.

SEQ ID NO. 86
AKG.Mtb-only_p0_sol1.
FVRPVAVDLTYIPVVGHALSARLYAENPSARDQILPVYAEYQQRSEKVLTEYNNKAALDQQRQW
ILHMAKLSAAMAKQAQYVAQLHVWARREHPTYERRTAPAPPWAKMWRHWVHALTRINLGLSPDE
KYELDLHARVRRNPRGSYQIAVVGLPAAPVTTAAMADPAADLIAPKTYCEELKGTDTGQACQIQ
MSDPAYNINISLPSYYPDQKSLENYIAQTRDKFLSAATSSTPREAPYELNITSATYQSAIPPRG
TQAVVLKVYQNAGGTHPTTTYKAFDWDQAYRKPITYDTLWQADTDPLPVVFPIVQGELSKQTGQ
QVSIAPNAGLDPVNYQNFAVTNDGVIFFFNPGELLPEAAGPTQVLVPRSAIDSMLAAAKNAAQQ
LVLSADNMREYLAAGAKERQRLATSLRNAAKMAVVGGALAYLVVKTLINATQLLKLLAKLQLPP
AATQTLAEAEVFATRYSGVSTANATVYMIDSVLMAETDTEQEIALFTVTNPPRTVSVSTLMDGR
IDHVELSARVAWMSESQLASEILVIADLARQKAQSAQYAFILDRMSQQVDADEHRVALLFIIDP
TISAIDGLYDLLGIGIPNQGGILYSSLEYFEKALEELAVHAASTRQALRSQFNDTLNVYLTAHN
ALGSSLHTAGVDLAKFFDPLTRGVLAEQVGGQSQLGVHTANATVYMIDTVLMATIADVLAEKEL
SHYNDIRAHTSVNAVNLEVLPAPEYSSAQRALSDADWHFIADPASRFYNLVLAQPFFDPSASFP
GKLNPDVNLVDTLNGGEYTVFAPTNAAFDKLPAATIDQLKTDAKLLSSILTYHVIAGQASPSRI
DGTHQTLNHIMPGEPNVAVKDLVRHFEQQVQPGRVVVMPWDRHIAAGTEISLDLLDPIYKRKVL
ELAAALQAETAVNTLFEKLEPMAAASKPPTPPMASKGTTTKKYMAADYDKLFRAHFADGWNTFN
LTRTPLMSQLIGWTADPIIGVQVRADRILALAVAASNNPELTTLTAALSGQLNPQVNLVDTLNS
GQYTVFAPTNAAFSKLPASTIDELKTNSSLLTSILTY
SEQ ID NO. 87
AKG.Mtb-only_p0_sol2.
MAADYDKLFRAHFADGWNTFNLSQFNDTLNVYLTAHNALGSSLHTAGVDLAKAVAASNNPELTT
LTAALSGQLNPQVNLVDTLNSGQYTVFAPTNAAFSKLPASTIDELKINSSLLTSILTYNHIMPG
EPNVAVKDLVRHFEQQVQPGRVVVMPWDRHIAAGTEISLDLLDPIYKRKVLELAAALFFDPLTR
GVLRRTAPAPPWAKMWRHWVHALTRINLGLSPDEKYELDLHARVRRNPRGSYQIAVVGLPAAPV
TTAAMADPAADLIAPKTYCEELKGTDTGQACQIQMSDPAYNINISLPSYYPDQKSLENYIAQTR
DKFLSAATSSTPREAPYELNITSATYQSAIPPRGTQAVVLKVYQNAGGTHPTTTYKAFDWDQAY
RKPITYDTLWQADTDPLPVVFPIVQGELSKQTGQQVSIAPNAGLDPVNYQNFAVTNDGVIFFFN
PGELLPEAAGPTQVLVPRSAIDSMLAGKLNPDVNLVDTLNGGEYTVFAPTNAAFDKLPAATIDQ
LKTDAKLLSSILTYHVIAGQASPSRIDGTHQTLAEQVGGQSQLGVSTANATVYMIDSVLMAETD
TEQEIALFTVTNPPRTVSVSTLMDGRIDHVELSARVAWMSESQLASEILVIADLARQKAQSAQY
AFILDRMSQQVDADEHRVALLGWTADPIIGVQVRADRILALRLYAENPSARDQILPVYAEYQQR
SEKVLTEYNNKAALDQQRQWILHMAKLSAAMAKQAQYVAQLHVWARREHPTYEAAKNAAQQLVL
SADNMREYLAAGAKERQRLATSLRNAAKATIADVLAEKELSHYNDIRAHTSVNAVNLEVLPAPE
YSSAQRALSDADWHFIADPASRFYNLVLAFVRPVAVDLTYIPVVGHALSAASKGTTTKKYQLPP
AATQTLTRTPLMSQLIFIIDPTISAIDGLYDLLGIGIPNQGGILYSSLEYFEKALEELAQPFFD
PSASFPMAVVGGALAYLVVKTLINATQLLKLLAKLQAETAVNTLFEKLEPMAAASKPPTPPMVH
AASTRQALRGVHTANATVYMIDTVLMAEAEVFATRYS
SEQ ID NO. 88
AKG.Mtb-only_p0_sol3.
WRHWVHALTRINLGLSPDEKYELDLHARVRRNPRGSYQIAVVGLMAADYDKLFRAHFADGWNTF
NLSQFNDTLNVYLTAHNALGSSLHTAGVDLAKFIIDPTISAIDGLYDLLGIGIPNQGGILYSSL
EYFEKALEELAAEQVGGQSQLGVSTANATVYMIDSVLMAETDTEQEIALFTVTNPPRTVSVSTL
MDGRIDHVELSARVAWMSESQLASEILVIADLARQKAQSAQYAFILDRMSQQVDADEHRVALLG
WTADPIIGVQVRADRILALRLYAENPSARDQILPVYAEYQQRSEKVLTEYNNKAALDQQRQWIL
HMAKLSAAMAKQAQYVAQLHVWARREHPTYEAPKTYCEELKGTDTGQACQIQMSDPAYNINISL
PSYYPDQKSLENYIAQTRDKFLSAATSSTPREAPYELNITSATYQSAIPPRGTQAVVLKVYQNA
GGTHPTTTYKAFDWDQAYRKPITYDTLWQADTDPLPVVFPIVQGELSKQTGQQVSIAPNAGLDP
VNYQNFAVTNDGVIFFFNPGELLPEAAGPTQVLVPRSAIDSMLAAAKNAAQQLVLSADNMREYL
AAGAKERQRLATSLRNAAKATIADVLAEKELSHYNDIRAHTSVNAVNLEVLPAPEYSSAQRALS
DADWHFIADPASRFYNLVLAQPFFDPSASFPMAVVGGALAYLVVKTLINATQLLKLLAKLQLPP
AATQTLFVRPVAVDLTYIPVVGHALSAQAETAVNTLFEKLEPMAAASKPPTPPMVHAASTRQAL
RASKGTTTKKYPAAPVTTAAMADPAADLIGVHTANATVYMIDTVLMAEAEVFATRYSGKLNPDV
NLVDTLNGGEYTVFAPTNAAFDKLPAATIDQLKTDAKLLSSILTYHVIAGQASPSRIDGTHQTL
AVAASNNPELTTLTAALSGQLNPQVNLVDTLNSGQYTVFAPTNAAFSKLPASTIDELKTNSSLL
TSILTYNHIMPGEPNVAVKDLVRHFEQQVQPGRVVVMPWDRHIAAGTEISLDLLDPIYKRKVLE
LAAALFFDPLTRGVLRRTAPAPPWAKMTRTPLMSQLI
SEQ ID NO. 89
AKG.Mtb-only_p0_sol4.
FFDPLTRGVLTRTPLMSQLIGWTADPIIGVQVRADRILALRLYAENPSARDQILPVYAEYQQRS
EKVLTEYNNKAALDQQRQWILHMAKLSAAMAKQAQYVAQLHVWARREHPTYEAAKNAAQQLVLS
ADNMREYLAAGAKERQRLATSLRNAAKATIADVLAEKELSHYNDIRAHTSVNAVNLEVLPAPEY
SSAQRALSDADWHFIADPASRFYNLVLAAHFADGWNTFNLMAADYDKLFRSQFNDTLNVYLTAH
NALGSSLHTAGVDLAKAVAASNNPELTTLTAALSGQLNPQVNLVDTLNSGQYTVFAPTNAAFSK
LPASTIDELKTNSSLLTSILTYNHIMPGEPNVAVKDLVRHFEQQVQPGRVVVMPWDRHIAAGTE
ISLDLLDPIYKRKVLELAAALRRTAPAPPWAKMWRHWVHALTRINLGLSPDEKYELDLHARVRR
NPRGSYQIAVVGLPAAPVTTAAMADPAADLIAPKTYCEELKGTDTGQACQIQMSDPAYNINISL
PSYYPDQKSLENYIAQTRDKFLSAATSSTPREAPYELNITSATYQSAIPPRGTQAVVLKVYQNA
GGTHPTTTYKAFDWDQAYRKPITYDTLWQADTDPLPVVFPIVQGELSKQTGQQVSIAPNAGLDP
VNYQNFAVTNDGVIFFFNPGELLPEAAGPTQVLVPRSAIDSMLAGKLNPDVNLVDTLNGGEYTV
FAPTNAAFDKLPAATIDQLKTDAKLLSSILTYHVIAGQASPSRIDGTHQTLAEQVGGQSQLGVS
TANATVYMIDSVLMQPFFDPSASFPAETDTEQEIALFTVTNPPRTVSVSTLMDGRIDHVELSAR
VAWMSESQLASEILVIADLARQKAQSAQYAFILDRMSQQVDADEHRVALLFIIDPTISAIDGLY
DLLGIGIPNQGGILYSSLEYFEKALEELAASKGTTTKKYQLPPAATQTLFVRPVAVDLTYIPVV
GHALSAMAVVGGALAYLVVKTLINATQLLKLLAKLQAETAVNTLFEKLEPMAAASKPPTPPMVH
AASTRQALRGVHTANATVYMIDTVLMAEAEVFATRYS
SEQ ID NO. 90
AKG.Mtb-only_p0_sol5.
FFDPLTRGVLTRTPLMSQLIAEQVGGQSQLGVSTANATVYMIDSVLMAETDTEQEIALFTVTNP
PRTVSVSTLMDGRIDHVELSARVAWMSESQLASEILVIADLARQKAQSAQYAFILDRMSQQVDA
DEHRVALLGWTADPIIGVQVRADRILALRLYAENPSARDQILPVYAEYQQRSEKVLTEYNNKAA
LDQQRQWILHMAKLSAAMAKQAQYVAQLHVWARREHPTYEAPKTYCEELKGTDTGQACQIQMSD
PAYNINISLPSYYPDQKSLENYIAQTRDKFLSAATSSTPREAPYELNITSATYQSAIPPRGTQA
VVLKVYQNAGGTHPTTTYKAFDWDQAYRKPITYDTLWQADTDPLPVVFPIVQGELSKQTGQQVS
IAPNAGLDPVNYQNFAVTNDGVIFFFNPGELLPEAAGPTQVLVPRSAIDSMLAAAKNAAQQLVL
SADNMREYLAAGAKERQRLATSLRNAAKAHFADGWNTFNLSQFNDTLNVYLTAHNALGSSLHTA
GVDLAKFIIDPTISAIDGLYDLLGIGIPNQGGILYSSLEYFEKALEELAATIADVLAEKELSHY
NDIRAHTSVNAVNLEVLPAPEYSSAQRALSDADWHFIADPASRFYNLVLAMAVVGGALAYLVVK
TLINATQLLKLLAKLQLPPAATQTLFVRPVAVDLTYIPVVGHALSAQAETAVNTLFEKLEPMAA
ASKPPTPPMVHAASTRQALRASKGTTTKKYPAAPVTTAAMADPAADLIGVHTANATVYMIDTVL
MAEAEVFATRYSGKLNPDVNLVDTLNGGEYTVFAPTNAAFDKLPAATIDQLKTDAKLLSSILTY
HVIAGQASPSRIDGTHQTLAVAASNNPELTTLTAALSGQLNPQVNLVDTLNSGQYTVFAPTNAA
FSKLPASTIDELKTNSSLLTSILTYNHIMPGEPNVAVKDLVRHFEQQVQPGRVVVMPWDRHIAA
GTEISLDLLDPIYKRKVLELAAALRRTAPAPPWAKMWRHWVHALTRINLGLSPDEKYELDLHAR
VRRNPRGSYQIAVVGLMAADYDKLFRQPFFDPSASFP
SEQ ID NO. 91
AKG.Mtb-only_p5_sol1.
WRHWVHALTRINLGLSPDEKYELDLHARVRRNPRGSYQIAVVGLQLPPAATQTLPAAPVTTAAM
ADPAADLIAPKTYCEELKGTDTGQACQIQMSDPAYNINISLPSYYPDQKSLENYIAQTRDKFLS
AATSSTPREAPYELNITSATYQSAIPPRGTQAVVLKVYQNAGGTHPTTTYKAFDWDQAYRKPIT
YDTLWQADTDPLPVVFPIVQGELSKQTGQQVSIAPNAGLDPVNYQNFAVTNDGVIFFFNPGELL
PEAAGPTQVLVPRSAIDSMLAAETDTEQEIALFTVTNPPRTVSVSTLMDGRIDHVELSARVAWM
SESQLASEILVIADLARQKAQSAQYAFILDRMSQQVDADEHRVALLGWTADPIIGVQVRADRIL
ALAVAASNNPELTTLTAALSGQLNPQVNLVDTLNSGQYTVFAPTNAAFSKLPASTIDELKINSS
LLTSILTYNHIMPGEPNVAVKDLVRHFEQQVQPGRVVVMPWDRHIAAGTEISLDLLDPIYKRKV
LELAAALQAETAVNTLFEKLEPMAAASKPPTPPMVHAASTRQALRAHFADGWNTFNLAEAEVFA
TRYSAAKNAAQQLVLSADNMREYLAAGAKERQRLATSLRNAAKRLYAENPSARDQILPVYAEYQ
QRSEKVLTEYNNKAALDQQRQWILHMAKLSAAMAKQAQYVAQLHVWARREHPTYEGVHTANATV
YMIDTVLMASKGTTTKKYMAADYDKLFRATIADVLAEKELSHYNDIRAHTSVNAVNLEVLPAPE
YSSAQRALSDADWHFIADPASRFYNLVLAFVRPVAVDLTYIPVVGHALSASQFNDTLNVYLTAH
NALGSSLHTAGVDLAKFFDPLTRGVLTRTPLMSQLIFIIDPTISAIDGLYDLLGIGIPNQGGIL
YSSLEYFEKALEELAAEQVGGQSQLGKLNPDVNLVDTLNGGEYTVFAPTNAAFDKLPAATIDQL
KTDAKLLSSILTYHVIAGQASPSRIDGTHQTLRRTAPAPPWAKMMAVVGGALAYLVVKTLINAT
QLLKLLAKLGVSTANATVYMIDSVLMQPFFDPSASFP
SEQ ID NO. 92
AKG.Mtb-only_p5_sol2.
APKTYCEELKGTDTGQACQIQMSDPAYNINISLPSYYPDQKSLENYIAQTRDKFLSAATSSTPR
EAPYELNITSATYQSAIPPRGTQAVVLKVYQNAGGTHPTTTYKAFDWDQAYRKPITYDTLWQAD
TDPLPVVFPIVQGELSKQTGQQVSIAPNAGLDPVNYQNFAVTNDGVIFFFNPGELLPEAAGPTQ
VLVPRSAIDSMLAAAKNAAQQLVLSADNMREYLAAGAKERQRLATSLRNAAKASKGTTTKKYQL
PPAATQTLFVRPVAVDLTYIPVVGHALSAAVAASNNPELTTLTAALSGQLNPQVNLVDTLNSGQ
YTVFAPTNAAFSKLPASTIDELKTNSSLLTSILTYNHIMPGEPNVAVKDLVRHFEQQVQPGRVV
VMPWDRHIAAGTEISLDLLDPIYKRKVLELAAALFFDPLTRGVLTRTPLMSQLIGWTADPIIGV
AASKPPTPPMRRTAPAPPWAKMWRHWVHALTRINLGLSPDEKYELDLHARVRRNPRGSYQIAVV
GLQAETAVNTLFEKLEPMAQVRADRILALRLYAENPSARDQILPVYAEYQQRSEKVLTEYNNKA
ALDQQRQWILHMAKLSAAMAKQAQYVAQLHVWARREHPTYEGKLNPDVNLVDTLNGGEYTVFAP
TNAAFDKLPAATIDQLKTDAKLLSSILTYHVIAGQASPSRIDGTHQTLAEQVGGQSQLPAAPVT
TAAMADPAADLIMAADYDKLFRSQFNDTLNVYLTAHNALGSSLHTAGVDLAKFIIDPTISAIDG
LYDLLGIGIPNQGGILYSSLEYFEKALEELAVHAASTRQALRGVHTANATVYMIDTVLMATIAD
VLAEKELSHYNDIRAHTSVNAVNLEVLPAPEYSSAQRALSDADWHFIADPASRFYNLVLAAHFA
DGWNTFNLAEAEVFATRYSAETDTEQEIALFTVTNPPRTVSVSTLMDGRIDHVELSARVAWMSE
SQLASEILVIADLARQKAQSAQYAFILDRMSQQVDADEHRVALLMAVVGGALAYLVVKTLINAT
QLLKLLAKLGVSTANATVYMIDSVLMQPFFDPSASFP
SEQ ID NO. 93
AKG.Mtb-only_p5_sol3.
WRHWVHALTRINLGLSPDEKYELDLHARVRRNPRGSYQIAVVGLQAETAVNTLFEKLEPMAGWT
ADPIIGVQLPPAATQTLFVRPVAVDLTYIPVVGHALSATRTPLMSQLIFIIDPTISAIDGLYDL
LGIGIPNQGGILYSSLEYFEKALEELAASKGTTTKKYAEAEVFATRYSGKLNPDVNLVDTLNGG
EYTVFAPTNAAFDKLPAATIDQLKTDAKLLSSILTYHVIAGQASPSRIDGTHQTLGVHTANATV
YMIDTVLMATIADVLAEKELSHYNDIRAHTSVNAVNLEVLPAPEYSSAQRALSDADWHFIADPA
SRFYNLVLAAHFADGWNTFNLPAAPVTTAAMADPAADLIAPKTYCEELKGTDTGQACQIQMSDP
AYNINISLPSYYPDQKSLENYIAQTRDKFLSAATSSTPREAPYELNITSATYQSAIPPRGTQAV
VLKVYQNAGGTHPTTTYKAFDWDQAYRKPITYDTLWQADTDPLPVVFPIVQGELSKQTGQQVSI
APNAGLDPVNYQNFAVTNDGVIFFFNPGELLPEAAGPTQVLVPRSAIDSMLAAETDTEQEIALF
TVTNPPRTVSVSTLMDGRIDHVELSARVAWMSESQLASEILVIADLARQKAQSAQYAFILDRMS
QQVDADEHRVALLSQFNDTLNVYLTAHNALGSSLHTAGVDLAKFFDPLTRGVLAVAASNNPELT
TLTAALSGQLNPQVNLVDTLNSGQYTVFAPTNAAFSKLPASTIDELKTNSSLLTSILTYNHIMP
GEPNVAVKDLVRHFEQQVQPGRVVVMPWDRHIAAGTEISLDLLDPIYKRKVLELAAALAASKPP
TPPMRRTAPAPPWAKMMAADYDKLFRAEQVGGQSQLQVRADRILALRLYAENPSARDQILPVYA
EYQQRSEKVLTEYNNKAALDQQRQWILHMAKLSAAMAKQAQYVAQLHVWARREHPTYEAAKNAA
QQLVLSADNMREYLAAGAKERQRLATSLRNAAKVHAASTRQALRMAVVGGALAYLVVKTLINAT
QLLKLLAKLGVSTANATVYMIDSVLMQPFFDPSASFP
SEQ ID NO. 94
AKG.Mtb-only_p5_sol4.
WRHWVHALTRINLGLSPDEKYELDLHARVRRNPRGSYQIAVVGLASKGTTTKKYPAAPVTTAAM
ADPAADLIMAADYDKLFRATIADVLAEKELSHYNDIRAHTSVNAVNLEVLPAPEYSSAQRALSD
ADWHFIADPASRFYNLVLAFVRPVAVDLTYIPVVGHALSAAVAASNNPELTTLTAALSGQLNPQ
VNLVDTLNSGQYTVFAPTNAAFSKLPASTIDELKTNSSLLTSILTYAETDTEQEIALFTVTNPP
RTVSVSTLMDGRIDHVELSARVAWMSESQLASEILVIADLARQKAQSAQYAFILDRMSQQVDAD
EHRVALLQVRADRILALRLYAENPSARDQILPVYAEYQQRSEKVLTEYNNKAALDQQRQWILHM
AKLSAAMAKQAQYVAQLHVWARREHPTYETRTPLMSQLIFIIDPTISAIDGLYDLLGIGIPNQG
GILYSSLEYFEKALEELAVHAASTRQALRAEQVGGQSQLAHFADGWNTFNLSQFNDTLNVYLTA
HNALGSSLHTAGVDLAKFFDPLTRGVLGWTADPIIGVQAETAVNTLFEKLEPMAAASKPPTPPM
RRTAPAPPWAKMAPKTYCEELKGTDTGQACQIQMSDPAYNINISLPSYYPDQKSLENYIAQTRD
KFLSAATSSTPREAPYELNITSATYQSAIPPRGTQAVVLKVYQNAGGTHPTTTYKAFDWDQAYR
KPITYDTLWQADTDPLPVVFPIVQGELSKQTGQQVSIAPNAGLDPVNYQNFAVTNDGVIFFFNP
GELLPEAAGPTQVLVPRSAIDSMLAAAKNAAQQLVLSADNMREYLAAGAKERQRLATSLRNAAK
MAVVGGALAYLVVKTLINATQLLKLLAKLQLPPAATQTLGVHTANATVYMIDTVLMAEAEVFAT
RYSGKLNPDVNLVDTLNGGEYTVFAPTNAAFDKLPAATIDQLKTDAKLLSSILTYHVIAGQASP
SRIDGTHQTLNHIMPGEPNVAVKDLVRHFEQQVQPGRVVVMPWDRHIAAGTEISLDLLDPIYKR
KVLELAAALGVSTANATVYMIDSVLMQPFFDPSASFP
SEQ ID NO. 95
AKG.Mtb-only_p5_sol5.
FFDPLTRGVLAASKPPTPPMWRHWVHALTRINLGLSPDEKYELDLHARVRRNPRGSYQIAVVGL
QLPPAATQTLTRTPLMSQLIRRTAPAPPWAKMAAKNAAQQLVLSADNMREYLAAGAKERQRLAT
SLRNAAKQPFFDPSASFPMAVVGGALAYLVVKTLINATQLLKLLAKLQAETAVNTLFEKLEPMA
DQQRQWILHMAKLSAAMAKQAQYVAQLHVWARREHPTYEGVHTANATVYMIDTVLMAEAEVFAT
RYSGKLNPDVNLVDTLNGGEYTVFAPTNAAFDKLPAATIDQLKTDAKLLSSILTYHVIAGQASP
SRIDGTHQTLAEQVGGQSQLPAAPVTTAAMADPAADLIMAADYDKLFRATIADVLAEKELSHYN
DIRAHTSVNAVNLEVLPAPEYSSAQRALSDADWHFIADPASRFYNLVLAFVRPVAVDLTYIPVV
GHALSARLYAENPSARDQILPVYAEYQQRSEKVLTEYNNKAALSQFNDTLNVYLTAHNALGSSL
HTAGVDLAKFIIDPTISAIDGLYDLLGIGIPNQGGILYSSLEYFEKALEELAVHAASTRQALRA
HFADGWNTFNLASKGTTTKKYAPKTYCEELKGTDTGQACQIQMSDPAYNINISLPSYYPDQKSL
ENYIAQTRDKFLSAATSSTPREAPYELNITSATYQSAIPPRGTQAVVLKVYQNAGGTHPTTTYK
AFDWDQAYRKPITYDTLWQADTDPLPVVFPIVQGELSKQTGQQVSIAPNAGLDPVNYQNFAVTN
DGVIFFFNPGELLPEAAGPTQVLVPRSAIDSMLAAETDTEQEIALFTVTNPPRTVSVSTLMDGR
IDHVELSARVAWMSESQLASEILVIADLARQKAQSAQYAFILDRMSQQVDADEHRVALLGWTAD
PIIGVQVRADRILALAVAASNNPELTTLTAALSGQLNPQVNLVDTLNSGQYTVFAPTNAAFSKL
PASTIDELKTNSSLLTSILTYNHIMPGEPNVAVKDLVRHFEQQVQPGRVVVMPWDRHIAAGTEI
SLDLLDPIYKRKVLELAAALGVSTANATVYMIDSVLM

Amino acid sequences for optimized solutions shown in Tables 53 and 54.

SEQ ID NO. 96. AKG.mixed_p0_sol1
FYVWDFAEKFKEDVINDFVSSYPHTSPPPLILKSWERNDPTQQISQATDPLSLLIETVTQALQA
LTIPSFIPEDFTFLSRIDGTHQTLVAELIGDALFVQLHAPLRGVARGQTLVLYIRMWNQAALAM
EVYQAETAVNTLFVAAWDKVMNLTYIPVVGHALSMAVVGGALAYLVVKTLINATQLLKLLAKLG
RASGVLPRYGLPPPWQPALRHPRSLFPEFSELFAAFPSFAGLRPTFDTRLMRSLLDAHIPQLVA
FHDQFVQALRAAIPAVVPELTPVAAAPPMLAVAATYDKGILTVYYQSGLSIVMDTRVATPSGLD
GPGMSTSAYDIGLFYRYAWQNPVFGGTHPTTTYKAFDWDQAYRKPITYIEKPVAPSVMILAAYH
PQQFIYAGSLSALLELENDNQLLYIMYNYPAMLGVHAASTRQALRVEMAAQQHVVIEAYTAAVE
LMPTQVELAMVRDGQLTIKAKLLSSILTYHGSVTPAVSQFNARTADGINYRVTLAIYHPQQFVY
SRFMTDPHAMRAAAVDIRETFRSELRALVEVYLLPLPNIPLLEYAARFITPVHPGYTATFLETP
SQFFPFTGLNSLTAARALPLTSLTSAAERGPGQMLDYVYPGHAIWWLARGLEFFQDGEQFGELL
FTNPTGAFDFPDSGTHSWEGFAIPIGQAMAGYTDDAGQTFVQELDEISTNIQAAEAFVAAYMPF
LVSSPDVLTTYIAAADEVSTQVAKELDEAVEAFIDELKTNSSLLTSILTYIAVMPPAPGMVRAV
PGRVVALFSRPGLPVEYLQVPSPSMGAAFQGAHARFAFPPEVHSAMLILAPQINFFYSAQAAAI
HEMFAFQYDGVNDFPKYPLNVFATANAIAGILFLHSGLIALADTDPLPVVFATIDVAIPKLRSQ
FNDTLNVYVRAATTMATYPISPPGFPSLPMYAGPGSASLRVLDALTDKLATIATFEMRYLQSLP
AHLRPGLDELYAMTLPPPVVAANRIQLLLLDAHIPQLIAVADPMGAAFDYAAEVLRVPPRPYVM
PHSPAAGFAAPVALAAYPITGKL
SEQ ID NO. 97. AKG.mixed_p0_sol2
FYVWDFAEKFKEDVINDFVSSYRVLDALTDKLATIATFEMRYLQSLPAHLRPGLDELLRVPPRP
YVMPHSPAAGFAAPVALAAYPITGKLQELDEISTNIGRASGVLPRYAFHDQFVQALRQAAEAFV
AAYMPFLGLPPPWQPALRHPRSLFPEFSELFAAFPSFAGLRPTFDTRLMRDYVYPGHAIWWLAR
GLEFFQDGEQFGELLFTNPTGAFLLDAHIPQLIAVADPMGAAFDYAAEVRAVPGRVVALATIDV
AIPKLRSQFNDTLNVYDFPDSGTHSWEAFPPEVHSAMLIEKPVAPSVMDTRVATPSGLDGPGMS
TSAYDIGLFYRYAWQNPVFAAADEVSTQVAAAAVDIRETFRGSVTPAVSQFNARTADGINYRVT
YIPVVGHALSMAVVGGALAYLVVKTLINATQLLKLLAKLPISPPGFPSLPYAMTLPPPVVAANR
IQLLVHAASTRQALRATYDKGILTVMYAGPGSASLVEMAAQQHVVIEAYTAAVELMPTQVELAS
LLDAHIPQLVILAPQINFFYILAAYHPQQFIYAGSLSALLLLPLPNIPLLEYAARFITPVHPGY
TATFLETPSQFFPFTGLNSLTMVRDGQLTIKAKLLSSILTYHAAIPAVVPELTPVAAAPPMLAV
ASQATDPLSLLIETVTQALQALTIPSFIPEDFTFLGFAIPIGQAMAGYTDDAGQTFVSRFMTDP
HAMRVAAWDKVMNLYYQSGLSIVMAARALPLTSLTSAAERGPGQMLIRMWNQAALAMEVYQAET
AVNTLFVAELIGDALFVQLHAPLRGVARGQTLVLYTLAIYHPQQFVYIAVMPPAPGMVAAFQGA
HARFVSSPDVLTTYIGGTHPTTTYKAFDWDQAYRKPITYIMYNYPAMLGFSRPGLPVEYLQVPS
PSMGSELRALVEVYIDELKINSSLLTSILTYPHTSPPPLILKSWERNDPTQQIVRAATTMATYE
LENDNQLLYSRIDGTHQTLSAQAAAIHEMFAFQYDGVNDFPKYPLNVFATANAIAGILFLHSGL
IALADTDPLPVVFKELDEAVEAF
SEQ ID NO. 98. AKG.mixed_p0_sol3
FYVWDFAEKFKEDVINDFVSSYAVADPMGAAFDYAAEVMYAGPGSASLQELDEISTNIVRAATT
MATYVSSPDVLTTYIYAMTLPPPVVAANRIQLLSLLDAHIPQLVILAPQINFFYTLAIYHPQQF
VYDTRVATPSGLDGPGMSTSAYDIGLFYRYAWQNPVFGGTHPTTTYKAFDWDQAYRKPITYIMY
NYPAMLGADTDPLPVVFATYDKGILTVMVRDGQLTIKDYVYPGHAIWWLARGLEFFQDGEQFGE
LLFTNPTGAFVAELIGDALFVQLHAPLRGVARGQTLVLYVAAWDKVMNLTYIPVVGHALSIAVM
PPAPGMVFSRPGLPVEYLQVPSPSMGSQFNDTLNVYELENDNQLLYSRIDGTHQTLGYTDDAGQ
TFVAAFQGAHARFAFPPEVHSAMLAAAVDIRETFRMAVVGGALAYLVVKTLINATQLLKLLAKL
ATIATFEMRYLQSLPAHLRPGLDELLRVPPRPYVMPHSPAAGRVLDALTDKLGLPPPWQPALRH
PRSLFPEFSELFAAFPSFAGLRPTFDTRLMRAAIPAVVPELTPVAAAPPMLAVASQATDPLSLL
IETVTQALQALTIPSFIPEDFTFLVHAASTRQALRVEMAAQQHVVIEAYTAAVELMPTQVELAA
AADEVSTQVAKELDEAVEAFIDELKTNSSLLTSILTYAKLLSSILTYHGSVTPAVSQFNARTAD
GINYRVGFAIPIGQAMAYYQSGLSIVMSAQAAAIHEMFDFPDSGTHSWERAVPGRVVALATIDV
AIPKLRSELRALVEVYPISPPGFPSLPWERNDPTQQIIEKPVAPSVMILAAYHPQQFIYAGSLS
ALLLLPLPNIPLLEYAARFITPVHPGYTATFLETPSQFFPFTGLNSLTAARALPLTSLTSAAER
GPGQMLIRMWNQAALAMEVYQAETAVNTLFAFQYDGVNDFPKYPLNVFATANAIAGILFLHSGL
IALSRFMTDPHAMRFAAPVALAAYPITGKLGRASGVLPRYLLDAHIPQLIPHTSPPPLILKSAF
HDQFVQALRQAAEAFVAAYMPFL
SEQ ID NO. 99. AKG.mixed_p0_sol4
FYVWDFAEKFKEDVINDFVSSYGSVTPAVSQFNARTADGINYRVTYIPVVGHALSIRMWNQAAL
AMEVYQAETAVNTLFVAAWDKVMNLAFPPEVHSAMLSQATDPLSLLIETVTQALQALTIPSFIP
EDFTFLAAAVDIRETFRMAVVGGALAYLVVKTLINATQLLKLLAKLGRASGVLPRYGLPPPWQP
ALRHPRSLFPEFSELFAAFPSFAGLRPTFDTRLMRSLLDAHIPQLVQAAEAFVAAYMPFLADTD
PLPVVFFAAPVALAAYPITGKLATIATFEMRYLQSLPAHLRPGLDELYAMTLPPPVVAANRIQL
LQELDEISTNIVSSPDVLITYIGGTHPTTTYKAFDWDQAYRKPITYIMYNYPAMLGFSRPGLPV
EYLQVPSPSMGIEKPVAPSVMILAAYHPQQFIYAGSLSALLVAELIGDALFVQLHAPLRGVARG
QTLVLYVEMAAQQHVVIEAYTAAVELMPTQVELAGYTDDAGQTFVSRFMTDPHAMRVRAATTMA
TYSAQAAAIHEMFAFQYDGVNDFPKYPLNVFATANAIAGILFLHSGLIALAAADEVSTQVAKEL
DEAVEAFLLPLPNIPLLEYAARFITPVHPGYTATFLETPSQFFPFTGLNSLTAARALPLTSLTS
AAERGPGQMLDYVYPGHAIWWLARGLEFFQDGEQFGELLFTNPTGAFYYQSGLSIVMDTRVATP
SGLDGPGMSTSAYDIGLFYRYAWQNPVFIDELKINSSLLTSILTYAKLLSSILTYHSELRALVE
VYPISPPGFPSLPDFPDSGTHSWEVHAASTRQALRILAPQINFFYTLAIYHPQQFVYIAVMPPA
PGMVATYDKGILTVMYAGPGSASLAAFQGAHARFGFAIPIGQAMAELENDNQLLYLRVPPRPYV
MPHSPAAGRVLDALTDKLAFHDQFVQALRAAIPAVVPELTPVAAAPPMLAVASRIDGTHQTLLL
DAHIPQLIAVADPMGAAFDYAAEVRAVPGRVVALSQFNDTLNVYPHTSPPPLILKSWERNDPTQ
QIMVRDGQLTIKATIDVAIPKLR
SEQ ID NO. 100. AKG.mixed_p0_sol5
LRVPPRPYVMPHSPAAGATIDVAIPKLRSQFNDTLNVYFAAPVALAAYPITGKLATIATFEMRY
LQSLPAHLRPGLDELATYDKGILTVSRIDGTHQTLVAELIGDALFVQLHAPLRGVARGQTLVLY
IRMWNQAALAMEVYQAETAVNTLFVAAWDKVMNLQAAEAFVAAYMPFLGLPPPWQPALRHPRSL
FPEFSELFAAFPSFAGLRPTFDTRLMRAFQYDGVNDFPKYPLNVFATANAIAGILFLHSGLIAL
RVLDALTDKLAFHDQFVQALRAKLLSSILTYHAAIPAVVPELTPVAAAPPMLAVAPHTSPPPLI
LKSGFAIPIGQAMAMYAGPGSASLSRFMTDPHAMRDYVYPGHAIWWLARGLEFFQDGEQFGELL
FTNPTGAFTLAIYHPQQFVYIEKPVAPSVMILAAYHPQQFIYAGSLSALLELENDNQLLYVHAA
STRQALRVEMAAQQHVVIEAYTAAVELMPTQVELASQATDPLSLLIETVTQALQALTIPSFIPE
DFTFLGRASGVLPRYYYQSGLSIVMMAVVGGALAYLVVKTLINATQLLKLLAKLAFPPEVHSAM
LVSSPDVLTTYIGGTHPTTTYKAFDWDQAYRKPITYIMYNYPAMLGVRAATTMATYLLPLPNIP
LLEYAARFITPVHPGYTATFLETPSQFFPFTGLNSLTDTRVATPSGLDGPGMSTSAYDIGLFYR
YAWQNPVFSAQAAAIHEMFIDELKTNSSLLTSILTYPISPPGFPSLPYAMTLPPPVVAANRIQL
LAAAVDIRETFRKELDEAVEAFDFPDSGTHSWELLDAHIPQLIADTDPLPVVFWERNDPTQQIA
ARALPLTSLTSAAERGPGQMLFYVWDFAEKFKEDVINDFVSSYAVADPMGAAFDYAAEVRAVPG
RVVALMVRDGQLTIKGYTDDAGQTFVAAFQGAHARFGSVTPAVSQFNARTADGINYRVTYIPVV
GHALSSLLDAHIPQLVAAADEVSTQVAQELDEISTNIILAPQINFFYIAVMPPAPGMVFSRPGL
PVEYLQVPSPSMGSELRALVEVY
SEQ ID NO. 101. AKG.mixed_p5 sol1
FYVWDFAEKFKEDVINDFVSSYAVADPMGAAFDYAAEVLRVPPRPYVMPHSPAAGAAIPAVVPE
LTPVAAAPPMLAVAIEKPVAPSVMDFPDSGTHSWERAVPGRVVALSLLDAHIPQLVRVLDALTD
KLADTDPLPVVFWERNDPTQQIVRAATTMATYVSSPDVLTTYIAAADEVSTQVAIDELKTNSSL
LTSILTYFAAPVALAAYPITGKLAAAVDIRETFRMAVVGGALAYLVVKTLINATQLLKLLAKLA
TIATFEMRYLQSLPAHLRPGLDELATIDVAIPKLRGGTHPTTTYKAFDWDQAYRKPITYIMYNY
PAMLGYYQSGLSIVMAARALPLTSLTSAAERGPGQMLVAELIGDALFVQLHAPLRGVARGQTLV
LYVEMAAQQHVVIEAYTAAVELMPTQVELADTRVATPSGLDGPGMSTSAYDIGLFYRYAWQNPV
FAFHDQFVQALRAKLLSSILTYHSELRALVEVYPISPPGFPSLPYAMTLPPPVVAANRIQLLSR
FMTDPHAMRGFAIPIGQAMAMYAGPGSASLATYDKGILTVGYTDDAGQTFVQELDEISTNIQAA
EAFVAAYMPFLILAPQINFFYILAAYHPQQFIYAGSLSALLELENDNQLLYDYVYPGHAIWWLA
RGLEFFQDGEQFGELLFTNPTGAFTLAIYHPQQFVYIAVMPPAPGMVFSRPGLPVEYLQVPSPS
MGAAFQGAHARFGRASGVLPRYGLPPPWQPALRHPRSLFPEFSELFAAFPSFAGLRPTFDTRLM
RSRIDGTHQTLVAAWDKVMNLTYIPVVGHALSIRMWNQAALAMEVYQAETAVNTLFSQATDPLS
LLIETVTQALQALTIPSFIPEDFTFLVHAASTRQALRSQFNDTLNVYLLPLPNIPLLEYAARFI
TPVHPGYTATFLETPSQFFPFTGLNSLTLLDAHIPQLISAQAAAIHEMFAFQYDGVNDFPKYPL
NVFATANAIAGILFLHSGLIALPHTSPPPLILKSMVRDGQLTIKGSVTPAVSQFNARTADGINY
RVAFPPEVHSAMLKELDEAVEAF
SEQ ID NO. 102. AKG.mixed_p5 sol2
IMYNYPAMLGVRAATTMATYPISPPGFPSLPYAMTLPPPVVAANRIQLLFSRPGLPVEYLQVPS
PSMGATYDKGILTVAARALPLTSLTSAAERGPGQMLFYVWDFAEKFKEDVINDFVSSYSRIDGT
HQTLGYTDDAGQTFVAAFQGAHARFAFPPEVHSAMLVEMAAQQHVVIEAYTAAVELMPTQVELA
MVRDGQLTIKSQATDPLSLLIETVTQALQALTIPSFIPEDFTFLDYVYPGHAIWWLARGLEFFQ
DGEQFGELLFTNPTGAFYYQSGLSIVMPHTSPPPLILKSSELRALVEVYVAELIGDALFVQLHA
PLRGVARGQTLVLYAAAVDIRETFRMAVVGGALAYLVVKTLINATQLLKLLAKLGLPPPWQPAL
RHPRSLFPEFSELFAAFPSFAGLRPTEDTRLMRGFAIPIGQAMASLLDAHIPQLVSAQAAAIHE
MFILAPQINFFYTLAIYHPQQFVYIAVMPPAPGMVRVLDALTDKLAFHDQFVQALRATIATFEM
RYLQSLPAHLRPGLDELRAVPGRVVALVHAASTRQALRSQFNDTLNVYELENDNQLLYAKLLSS
ILTYHGSVTPAVSQFNARTADGINYRVSREMTDPHAMRVAAWDKVMNLTYIPVVGHALSIRMWN
QAALAMEVYQAETAVNTLFAFQYDGVNDFPKYPLNVFATANAIAGILFLHSGLIALADTDPLPV
VFWERNDPTQQILLDAHIPQLIAVADPMGAAFDYAAEVLRVPPRPYVMPHSPAAGATIDVAIPK
LRGGTHPTTTYKAFDWDQAYRKPITYIEKPVAPSVMILAAYHPQQFIYAGSLSALLDFPDSGTH
SWEAAIPAVVPELTPVAAAPPMLAVAMYAGPGSASLQELDEISTNIGRASGVLPRYQAAEAFVA
AYMPFLVSSPDVLTTYIAAADEVSTQVAKELDEAVEAFLLPLPNIPLLEYAARFITPVHPGYTA
TFLETPSQFFPFTGLNSLTDTRVATPSGLDGPGMSTSAYDIGLFYRYAWQNPVFIDELKINSSL
LTSILTYFAAPVALAAYPITGKL
SEQ ID NO. 103. AKG.mixed_p5 sol3
FYVWDFAEKFKEDVINDFVSSYMYAGPGSASLVEMAAQQHVVIEAYTAAVELMPTQVELAGYTD
DAGQTFVAAFQGAHARFSQATDPLSLLIETVTQALQALTIPSFIPEDFTFLAVADPMGAAFDYA
AEVLRVPPRPYVMPHSPAAGAFPPEVHSAMLATIDVAIPKLRSQFNDTLNVYPISPPGFPSLPS
RIDGTHQTLIRMWNQAALAMEVYQAETAVNTLESAQAAAIHEMFIDELKTNSSLLTSILTYAAA
VDIRETFRMAVVGGALAYLVVKTLINATQLLKLLAKLGLPPPWQPALRHPRSLFPEFSELFAAF
PSFAGLRPTFDTRLMRAFQYDGVNDFPKYPLNVFATANAIAGILFLHSGLIALELENDNQLLYV
AAWDKVMNLTYIPVVGHALSMVRDGQLTIKKELDEAVEAFVRAATTMATYVSSPDVLTTYIWER
NDPTQQIAARALPLTSLTSAAERGPGQMLDYVYPGHAIWWLARGLEFFQDGEQFGELLFTNPTG
AFPHTSPPPLILKSGRASGVLPRYGFAIPIGQAMAFSRPGLPVEYLQVPSPSMGQELDEISTNI
QAAEAFVAAYMPFLAAADEVSTQVAVHAASTRQALRIEKPVAPSVMDTRVATPSGLDGPGMSTS
AYDIGLFYRYAWQNPVFAFHDQFVQALRAAIPAVVPELTPVAAAPPMLAVAATYDKGILTVYYQ
SGLSIVMSLLDAHIPQLVGGTHPTTTYKAFDWDQAYRKPITYRVLDALTDKLATIATFEMRYLQ
SLPAHLRPGLDELYAMTLPPPVVAANRIQLLIMYNYPAMLGILAPQINFFYTLAIYHPQQFVYS
RFMTDPHAMRLLDAHIPQLIADTDPLPVVFFAAPVALAAYPITGKLVAELIGDALFVQLHAPLR
GVARGQTLVLYIAVMPPAPGMVRAVPGRVVALAKLLSSILTYHGSVTPAVSQFNARTADGINYR
VILAAYHPQQFIYAGSLSALLLLPLPNIPLLEYAARFITPVHPGYTATFLETPSQFFPFTGLNS
LTDFPDSGTHSWESELRALVEVY
SEQ ID NO. 104. AKG.mixed_p5 sol4
FYVWDFAEKFKEDVINDFVSSYYYQSGLSIVMVEMAAQQHVVIEAYTAAVELMPTQVELAMVRD
GQLTIKDTRVATPSGLDGPGMSTSAYDIGLFYRYAWQNPVFGSVTPAVSQFNARTADGINYRVQ
AAEAFVAAYMPFLILAPQINFFYILAAYHPQQFIYAGSLSALLLLPLPNIPLLEYAARFITPVH
PGYTATFLETPSQFFPFTGLNSLTAARALPLTSLTSAAERGPGQMLWERNDPTQQIIEKPVAPS
VMVSSPDVLTTYIGGTHPTTTYKAFDWDQAYRKPITYRVLDALTDKLATIATFEMRYLQSLPAH
LRPGLDELLRVPPRPYVMPHSPAAGAFPPEVHSAMLATIDVAIPKLRFAAPVALAAYPITGKLA
DTDPLPVVFYAMTLPPPVVAANRIQLLSRFMTDPHAMRVHAASTRQALRATYDKGILTVTYIPV
VGHALSAAAVDIRETFRMAVVGGALAYLVVKTLINATQLLKLLAKLGYTDDAGQTFVAAFQGAH
ARFSELRALVEVYVAELIGDALFVQLHAPLRGVARGQTLVLYIRMWNQAALAMEVYQAETAVNT
LFAFQYDGVNDFPKYPLNVFATANAIAGILFLHSGLIALIMYNYPAMLGVRAATTMATYPISPP
GFPSLPELENDNQLLYAKLLSSILTYHSQATDPLSLLIETVTQALQALTIPSFIPEDFTFLGFA
IPIGQAMAAVADPMGAAFDYAAEVDFPDSGTHSWERAVPGRVVALMYAGPGSASLQELDEISTN
IGRASGVLPRYGLPPPWQPALRHPRSLFPEFSELFAAFPSFAGLRPTFDTRLMRDYVYPGHAIW
WLARGLEFFQDGEQFGELLFTNPTGAFTLAIYHPQQFVYIDELKTNSSLLTSILTYPHTSPPPL
ILKSAFHDQFVQALRAAIPAVVPELTPVAAAPPMLAVAIAVMPPAPGMVFSRPGLPVEYLQVPS
PSMGSQFNDTLNVYLLDAHIPQLISAQAAAIHEMFVAAWDKVMNLSRIDGTHQTLSLLDAHIPQ
LVAAADEVSTQVAKELDEAVEAF
SEQ ID NO. 105. AKG.mixed_p5 sol5
FYVWDFAEKFKEDVINDFVSSYKELDEAVEAFELENDNQLLYIMYNYPAMLGYYQSGLSIVMAA
RALPLTSLTSAAERGPGQMLIRMWNQAALAMEVYQAETAVNTLFAAAVDIRETFRSQFNDTLNV
YAKLLSSILTYHGSVTPAVSQFNARTADGINYRVILAAYHPQQFIYAGSLSALLPHTSPPPLIL
KSSRFMTDPHAMRMVRDGQLTIKGFAIPIGQAMAVAAWDKVMNLTYIPVVGHALSIAVMPPAPG
MVRAVPGRVVALFAAPVALAAYPITGKLATIATFEMRYLQSLPAHLRPGLDELGRASGVLPRYG
LPPPWQPALRHPRSLFPEFSELFAAFPSFAGLRPTFDTRLMRSQATDPLSLLIETVTQALQALT
IPSFIPEDFTFLAAFQGAHARFAFHDQFVQALRAAIPAVVPELTPVAAAPPMLAVAAVADPMGA
AFDYAAEVLRVPPRPYVMPHSPAAGGYTDDAGQTFVQELDEISTNIVRAATTMATYPISPPGFP
SLPDFPDSGTHSWEAFQYDGVNDFPKYPLNVFATANAIAGILFLHSGLIALRVLDALTDKLILA
PQINFFYTLAIYHPQQFVYATYDKGILTVMYAGPGSASLSRIDGTHQTLDYVYPGHAIWWLARG
LEFFQDGEQFGELLFTNPTGAFVHAASTRQALRVEMAAQQHVVIEAYTAAVELMPTQVELALLD
AHIPQLIQAAEAFVAAYMPFLAAADEVSTQVADTRVATPSGLDGPGMSTSAYDIGLFYRYAWQN
PVFIDELKTNSSLLTSILTYATIDVAIPKLRADTDPLPVVFYAMTLPPPVVAANRIQLLFSRPG
LPVEYLQVPSPSMGSELRALVEVYLLPLPNIPLLEYAARFITPVHPGYTATFLETPSQFFPFTG
LNSLTVAELIGDALFVQLHAPLRGVARGQTLVLYMAVVGGALAYLVVKTLINATQLLKLLAKLA
FPPEVHSAMLWERNDPTQQIIEKPVAPSVMSLLDAHIPQLVSAQAAAIHEMFVSSPDVLTTYIG
GTHPTTTYKAFDWDQAYRKPITY
SEQ ID NO. 204. Human Polyubiquitin-B (UBB), N terminus with G76A mutation, UniProt
POCG47, aa 1-76. G76A mutation underlined.
MQIFVKTLTGKTITLEVEPSDTIENVKAKIQDKEGIPPDQQRLIFAGKQLEDGRTLSDYNIQKE
STLHLVLRLRGA
SEQ ID NO. 205. Human Polyubiquitin-B (UBB), N terminus with G76GR mutation, UniProt
POCG47, aa 1-76. The addition of GR after glycine at residue 76 (G76GR) underlined.
MQIFVKTLTGKTITLEVEPSDTIENVKAKIQDKEGIPPDQQRLIFAGKQLEDGRTLSDYNIQKE
STLHLVLRLRGGR
SEQ ID NO. 206. Human IkBalpha polyUb destruction motif, UniProt P25963, aa 23-41.
MERLLDDRHDSGLDSMKDEE
SEQ ID NO. 207. An example of an “Mtb-only” cassette with the G76A ubiquitin attached to the
N-terminus (ubiquitin is underlined). Refer to SEQ ID NO. 91 for the polyepitope portion of the
cassette.
MQIFVKTLTGKTITLEVEPSDTIENVKAKIQDKEGIPPDQQRLIFAGKQLEDGRTLSDYNIQKE
STLHLVLRLRGAWRHWVHALTRINLGLSPDEKYELDLHARVRRNPRGSYQIAVVGLQLPPAATQ
TLPAAPVTTAAMADPAADLIAPKTYCEELKGTDTGQACQIQMSDPAYNINISLPSYYPDQKSLE
NYIAQTRDKFLSAATSSTPREAPYELNITSATYQSAIPPRGTQAVVLKVYQNAGGTHPTTTYKA
FDWDQAYRKPITYDTLWQADTDPLPVVFPIVQGELSKQTGQQVSIAPNAGLDPVNYQNFAVTND
GVIFFFNPGELLPEAAGPTQVLVPRSAIDSMLAAETDTEQEIALFTVTNPPRTVSVSTLMDGRI
DHVELSARVAWMSESQLASEILVIADLARQKAQSAQYAFILDRMSQQVDADEHRVALLGWTADP
IIGVQVRADRILALAVAASNNPELTTLTAALSGQLNPQVNLVDTLNSGQYTVFAPTNAAFSKLP
ASTIDELKTNSSLLTSILTYNHIMPGEPNVAVKDLVRHFEQQVQPGRVVVMPWDRHIAAGTEIS
LDLLDPIYKRKVLELAAALQAETAVNTLFEKLEPMAAASKPPTPPMVHAASTRQALRAHFADGW
NTFNLAEAEVFATRYSAAKNAAQQLVLSADNMREYLAAGAKERQRLATSLRNAAKRLYAENPSA
RDQILPVYAEYQQRSEKVLTEYNNKAALDQQRQWILHMAKLSAAMAKQAQYVAQLHVWARREHP
TYEGVHTANATVYMIDTVLMASKGTTTKKYMAADYDKLFRATIADVLAEKELSHYNDIRAHTSV
NAVNLEVLPAPEYSSAQRALSDADWHFIADPASRFYNLVLAFVRPVAVDLTYIPVVGHALSASQ
FNDTLNVYLTAHNALGSSLHTAGVDLAKFFDPLTRGVLTRTPLMSQLIFIIDPTISAIDGLYDL
LGIGIPNQGGILYSSLEYFEKALEELAAEQVGGQSQLGKLNPDVNLVDTLNGGEYTVFAPTNAA
FDKLPAATIDQLKTDAKLLSSILTYHVIAGQASPSRIDGTHQTLRRTAPAPPWAKMMAVVGGAL
AYLVVKTLINATQLLKLLAKLGVSTANATVYMIDSVLMQPFFDPSASFP
SEQ ID NO. 208. An second example of an “Mtb-only” cassette with the G76A ubiquitin
attached to the N-terminus (ubiquitin is underlined). Refer to SEQ ID NO. 92 for the polyepitope
portion of the cassette.
MQIFVKTLTGKTITLEVEPSDTIENVKAKIQDKEGIPPDQQRLIFAGKQLEDGRTLSDYNIQKE
STLHLVLRLRGAAPKTYCEELKGTDTGQACQIQMSDPAYNINISLPSYYPDQKSLENYIAQTRD
KFLSAATSSTPREAPYELNITSATYQSAIPPRGTQAVVLKVYQNAGGTHPTTTYKAFDWDQAYR
KPITYDTLWQADTDPLPVVFPIVQGELSKQTGQQVSIAPNAGLDPVNYQNFAVINDGVIFFFNP
GELLPEAAGPTQVLVPRSAIDSMLAAAKNAAQQLVLSADNMREYLAAGAKERQRLATSLRNAAK
ASKGTTTKKYQLPPAATQTLFVRPVAVDLTYIPVVGHALSAAVAASNNPELTTLTAALSGQLNP
QVNLVDTLNSGQYTVFAPTNAAFSKLPASTIDELKTNSSLLTSILTYNHIMPGEPNVAVKDLVR
HFEQQVQPGRVVVMPWDRHIAAGTEISLDLLDPIYKRKVLELAAALFFDPLTRGVLTRTPLMSQ
LIGWTADPIIGVAASKPPTPPMRRTAPAPPWAKMWRHWVHALTRINLGLSPDEKYELDLHARVR
RNPRGSYQIAVVGLQAETAVNTLFEKLEPMAQVRADRILALRLYAENPSARDQILPVYAEYQQR
SEKVLTEYNNKAALDQQRQWILHMAKLSAAMAKQAQYVAQLHVWARREHPTYEGKLNPDVNLVD
TLNGGEYTVFAPTNAAFDKLPAATIDQLKTDAKLLSSILTYHVIAGQASPSRIDGTHQTLAEQV
GGQSQLPAAPVTTAAMADPAADLIMAADYDKLFRSQFNDTLNVYLTAHNALGSSLHTAGVDLAK
FIIDPTISAIDGLYDLLGIGIPNQGGILYSSLEYFEKALEELAVHAASTRQALRGVHTANATVY
MIDTVLMATIADVLAEKELSHYNDIRAHTSVNAVNLEVLPAPEYSSAQRALSDADWHFIADPAS
RFYNLVLAAHFADGWNTFNLAEAEVFATRYSAETDTEQEIALFTVTNPPRTVSVSTLMDGRIDH
VELSARVAWMSESQLASEILVIADLARQKAQSAQYAFILDRMSQQVDADEHRVALLMAVVGGAL
AYLVVKTLINATQLLKLLAKLGVSTANATVYMIDSVLMQPFFDPSASFP
SEQ ID NO. 209. An example of a “Mixed” cassette with the G76A ubiquitin attached to the N-
terminus (ubiquitin is underlined). Refer to SEQ ID NO. 101 for the polyepitope portion of the
cassette.
MQIFVKTLTGKTITLEVEPSDTIENVKAKIQDKEGIPPDQQRLIFAGKQLEDGRTLSDYNIQKE
STLHLVLRLRGAFYVWDFAEKFKEDVINDFVSSYAVADPMGAAFDYAAEVLRVPPRPYVMPHSP
AAGAAIPAVVPELTPVAAAPPMLAVAIEKPVAPSVMDFPDSGTHSWERAVPGRVVALSLLDAHI
PQLVRVLDALTDKLADTDPLPVVFWERNDPTQQIVRAATTMATYVSSPDVLTTYIAAADEVSTQ
VAIDELKTNSSLLTSILTYFAAPVALAAYPITGKLAAAVDIRETFRMAVVGGALAYLVVKTLIN
ATQLLKLLAKLATIATFEMRYLQSLPAHLRPGLDELATIDVAIPKLRGGTHPTTTYKAFDWDQA
YRKPITYIMYNYPAMLGYYQSGLSIVMAARALPLTSLTSAAERGPGQMLVAELIGDALFVQLHA
PLRGVARGQTLVLYVEMAAQQHVVIEAYTAAVELMPTQVELADTRVATPSGLDGPGMSTSAYDI
GLFYRYAWQNPVFAFHDQFVQALRAKLLSSILTYHSELRALVEVYPISPPGFPSLPYAMTLPPP
VVAANRIQLLSRFMTDPHAMRGFAIPIGQAMAMYAGPGSASLATYDKGILTVGYTDDAGQTFVQ
ELDEISTNIQAAEAFVAAYMPFLILAPQINFFYILAAYHPQQFIYAGSLSALLELENDNQLLYD
YVYPGHAIWWLARGLEFFQDGEQFGELLFTNPTGAFTLAIYHPQQFVYIAVMPPAPGMVFSRPG
LPVEYLQVPSPSMGAAFQGAHARFGRASGVLPRYGLPPPWQPALRHPRSLFPEFSELFAAFPSF
AGLRPTFDTRLMRSRIDGTHQTLVAAWDKVMNLTYIPVVGHALSIRMWNQAALAMEVYQAETAV
NTLFSQATDPLSLLIETVTQALQALTIPSFIPEDFTFLVHAASTRQALRSQFNDTLNVYLLPLP
NIPLLEYAARFITPVHPGYTATFLETPSQFFPFTGLNSLTLLDAHIPQLISAQAAAIHEMFAFQ
YDGVNDFPKYPLNVFATANAIAGILFLHSGLIALPHTSPPPLILKSMVRDGQLTIKGSVTPAVS
QFNARTADGINYRVAFPPEVHSAMLKELDEAVEAF
SEQ ID NO. 210. An example of a “Mixed” cassette with the G76A ubiquitin attached to the N-
terminus (ubiquitin is underlined). Refer to SEQ ID NO. 102 for the polyepitope portion of the
cassette.
MQIFVKTLTGKTITLEVEPSDTIENVKAKIQDKEGIPPDQQRLIFAGKQLEDGRTLSDYNIQKE
STLHLVLRLRGAIMYNYPAMLGVRAATTMATYPISPPGFPSLPYAMTLPPPVVAANRIQLLFSR
PGLPVEYLQVPSPSMGATYDKGILTVAARALPLTSLTSAAERGPGQMLFYVWDFAEKFKEDVIN
DFVSSYSRIDGTHQTLGYTDDAGQTFVAAFQGAHARFAFPPEVHSAMLVEMAAQQHVVIEAYTA
AVELMPTQVELAMVRDGQLTIKSQATDPLSLLIETVTQALQALTIPSFIPEDFTFLDYVYPGHA
IWWLARGLEFFQDGEQFGELLFTNPTGAFYYQSGLSIVMPHTSPPPLILKSSELRALVEVYVAE
LIGDALFVQLHAPLRGVARGQTLVLYAAAVDIRETFRMAVVGGALAYLVVKTLINATQLLKLLA
KLGLPPPWQPALRHPRSLFPEFSELFAAFPSFAGLRPTEDTRLMRGFAIPIGQAMASLLDAHIP
QLVSAQAAAIHEMFILAPQINFFYTLAIYHPQQFVYIAVMPPAPGMVRVLDALTDKLAFHDQFV
QALRATIATFEMRYLQSLPAHLRPGLDELRAVPGRVVALVHAASTRQALRSQFNDTLNVYELEN
DNQLLYAKLLSSILTYHGSVTPAVSQFNARTADGINYRVSRFMTDPHAMRVAAWDKVMNLTYIP
VVGHALSIRMWNQAALAMEVYQAETAVNTLFAFQYDGVNDFPKYPLNVFATANAIAGILFLHSG
LIALADTDPLPVVFWERNDPTQQILLDAHIPQLIAVADPMGAAFDYAAEVLRVPPRPYVMPHSP
AAGATIDVAIPKLRGGTHPTTTYKAFDWDQAYRKPITYIEKPVAPSVMILAAYHPQQFIYAGSL
SALLDFPDSGTHSWEAAIPAVVPELTPVAAAPPMLAVAMYAGPGSASLQELDEISTNIGRASGV
LPRYQAAEAFVAAYMPFLVSSPDVLTTYIAAADEVSTQVAKELDEAVEAFLLPLPNIPLLEYAA
RFITPVHPGYTATFLETPSQFFPFTGLNSLTDTRVATPSGLDGPGMSTSAYDIGLFYRYAWQNP
VFIDELKINSSLLTSILTYFAAPVALAAYPITGKL

### Example 34: Impact of Signal Peptide on HLA Class II-Directed Mtb mRNA LNPs and Comparison of ALC-0315 Formulation to KC3-OA/DPPS LNP Formulation

The aim of this study was to explore the impact of the signal peptide on the immunogenicity of an HLA-II directed mRNA and compare this immunogenicity to an ALC-0315 LNP formulation. The DPPS-NH₄, PEG-DMG and KC3-OA were kept constant at 5 mol %, 1.5 mol %, and 48 mol % respectively with an N/P ratio of 5.25 using mRNA encoding for concatenated Mtb proteins paired with four unique signal peptides. All mRNAs used N1-methylpseudouridine (N1MeU). The antigen encoded by the four mRNA constructs consisted of 7 Mtb proteins and 10 minimal epitopes (polypeptide sequence corresponds to SEQ ID NO. 20) separated by spacers: 5 proteins were included in their entirety (ExsH, EsxW, EsxV, EsxA, EsxB), 2 in partial (Ag85B, Mtb39a), and a set of ten 15-mer epitopes were encoded at the 3′ end. The mRNAs only differed in their 5′ signal peptide and 3′ transmembrane/cytoplasmic domains: the 1^{st} construct used an HLA-I signal peptide and terminal domain (sec/MITD; SEQ ID NOs. 37 and 38); the 2nd construct used a human LAMP-1signal peptide and terminal domain (SEQ ID NOs. 39 and 40); the 3^{rd} construct used the signal peptide from HLA-DRa (SEQ ID NOs. 41 and 42); and the 4^{th} construct used human tissue plasminogen activator (tPA) signal peptide (SEQ ID NOs. 43 and 44). Characterizations of the mRNA-LNPs are shown below in Table 55. The DSPC and cholesterol were also kept constant at 5 mol % and 40.5 mol %, respectively in the KC3OA LNP formulations. An ALC-0315 based formulation composed of 46.3 mol % ALC-0315, 9.4 mol % DSPC, 42.7 mol % cholesterol and 1.56 mol % PEG-DMG with a N/P of 6.2 was also made and evaluated.

LNPs were prepared as described in Example 9, characterized for particle size and zeta potential as described in Example 10, mRNA encapsulation efficiency and mRNA concentration as described in Example 10 and evaluated for antigen-specific CD4- and CD8-T cell responses in the spleens of CB6F1 mice.

TABLE 55
Physicochemical properties and characterization of ALC-0315 and 5 mol % DPPS-
targeted KC3-OA LNPs. Particle size refers to the Z-Average particle size.
Post-F/T refers to samples that were measured after a freeze/thaw event,
and Zeta refers to zeta potential measurements at either pH 5 or pH 7 in
units of mV. mRNA encapsulation efficiency is referred to as (E.E, %).

| | | | Particle Size | | | |
|---|---|---|---|---|---|---|
| | | Particle | (nm) Post- | | Zeta, | Zeta, |
| ICL | mRNA | size (nm) | F/T | E.E, % | pH 5 | pH 7 |
| ALC-0315 | SEQ ID NOs. 37 | 81.28 | 82.11 | 85.0 ± 6.4 | 5.03 | −1.81 |
| | and 38 | | | | | |
| ALC-0315 | SEQ ID NOs. 39 | 83.90 | 81.19 | 84.6 ± 6.0 | 5.80 | −2.13 |
| | and 40 | | | | | |
| ALC-0315 | SEQ ID NOs. | 78.68 | 79.05 | 85.1 ± 4.6 | 7.34 | −4.08 |
| | 41 and 42 | | | | | |
| ALC-0315 | SEQ ID Nos. 43 | 81.86 | 82.22 | 82.9 ± 1.9 | 5.77 | −2.56 |
| | and 44 | | | | | |
| KC3-OA | SEQ ID NOs. | 104.30 | 108.80 | 91.4 ± 7.3 | 13.75 | −1.78 |
| | 37 and 38 | | | | | |
| KC3-OA | SEQ ID NOs. | 105.10 | 109.60 | 92.1 ± 0.8 | 15.93 | −3.06 |
| | 39 and 40 | | | | | |
| KC3-OA | SEQ ID NOs. | 106.20 | 110.00 | 92.3 ± 5.3 | 15.19 | −3.04 |
| | 41 and 42 | | | | | |
| KC3-OA | SEQ ID NOs. | 102.00 | 107.10 | 91.9 ± 7.1 | 14.92 | −1.15 |
| | 43 and 44 | | | | | |

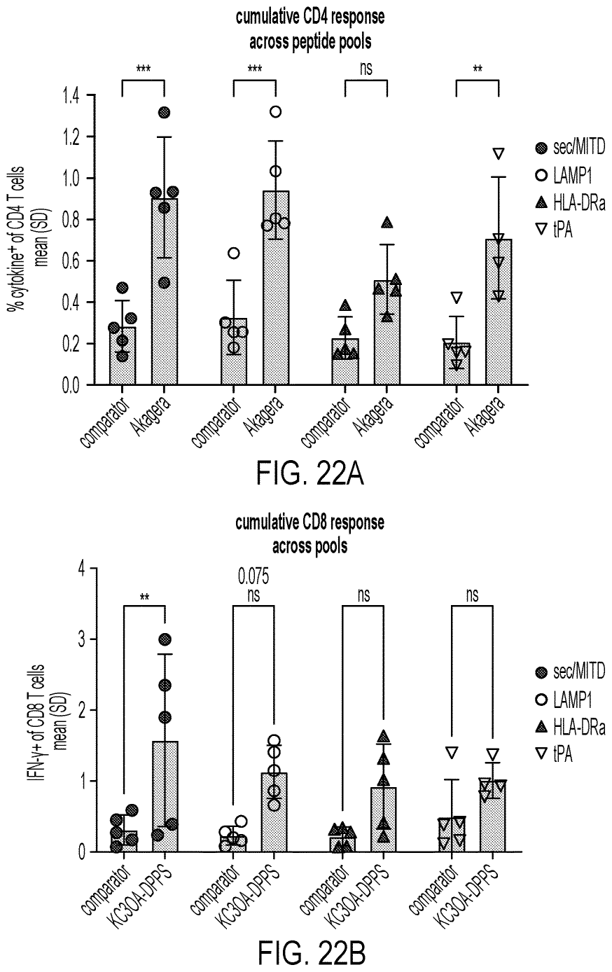

Immunogenicity of mRNA-LNPs vaccines encoding Mtb antigens in CB6F1 mice (FIG. 22). CB6F1 female mice, ages 6-8 weeks old, were immunized intramuscularly in the caudal thigh with 1 μg mRNA-LNP. 6 weeks later, mice were given a homologous boost and spleens were harvested 7 days later. Splenic cells were stimulated with overlapping peptide pools for 6 h with brefeldin A added at +1 h. Peptide pools consisted of 15mer peptides overlapping by 11 amino acids and covered each Mtb protein encoded by the mRNA. Cells were stained for extracellular markers, fixed and permeabilized, and stained for intracellular IFN-γ, TNF-α and IL-2. (FIG. 22A) Mtb-specific CD4 T cells were defined as any cell that produced either of these 3 cytokines following peptide stimulation. (FIG. 22B) Mtb-specific CD8 T cells were identified as any cell that produced IFN-γ; TNF-α and IL-2 producing CD8 T cells were found within the IFN-γ-producing population. Cumulative T cell responses were calculated by adding the % of T cells producing cytokines to each of the Mtb proteins; the % of cytokine⁺ T cells in the unstimulated negative control well was subtracted from the cumulative total. These data demonstrated that the total CD4 and CD8 T-cell responses were greater for the sec/MITD (SEQ ID NOs. 37 and 38) and LAMP-1 (SEQ ID NOs. 39 and 40) containing constructs. In addition, the KC3-OA/DPPS LNPs outperformed ALC-0315 LNPS with respect to both CD4 and CD8 T-cell immunogenicity.

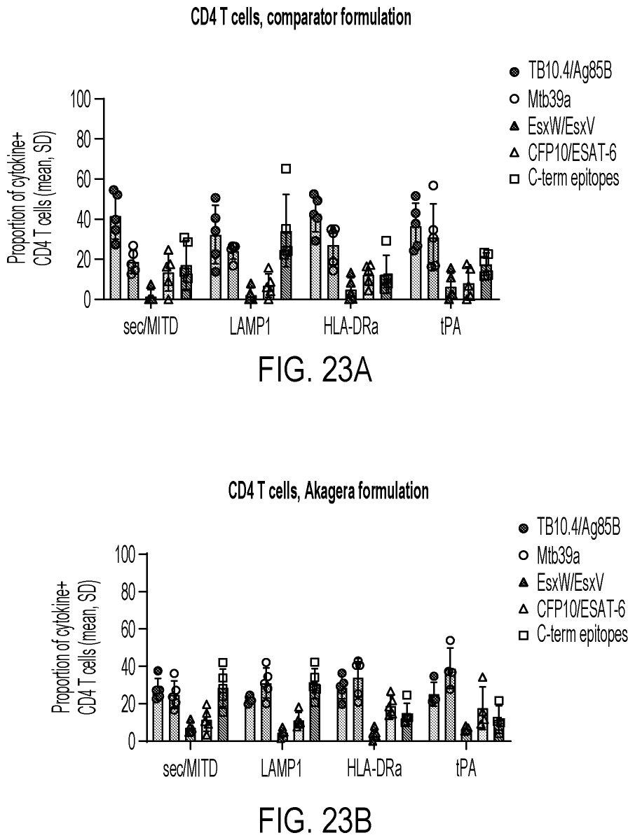

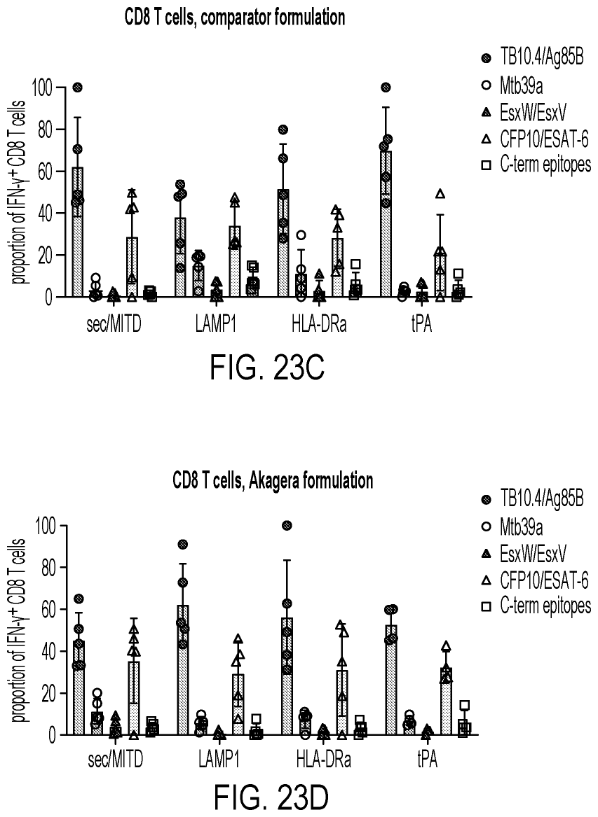

The proportion of total vaccine-induced T cell response to individual or subsets of Mtb antigens was also evaluated (FIGS. 23A-23D). Data correspond to cumulative T cell responses shown in FIG. 22 and are normalized to 100%. Distribution of T cell responses across antigens and mRNA constructs using the sec/MITD, LAMP-1, HLA-DRα, and tPA N-terminal signal sequences and transmembrane/cytoplasmic domains. The profile of CD4 T cell responses using the ALC-0315 comparator (FIG. 23A) or KC3-OA/DPPS LNP formulation (FIG. 23B). The profile of CD8 T cell responses using the ALC-0315 comparator (FIG. 23C) or KC3-OA/DPPS LNP formulation (FIG. 23D).

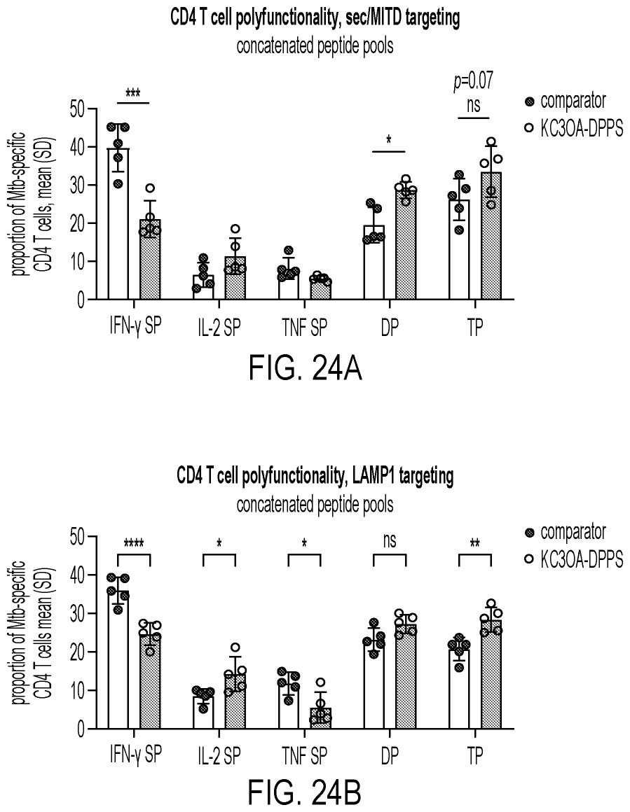

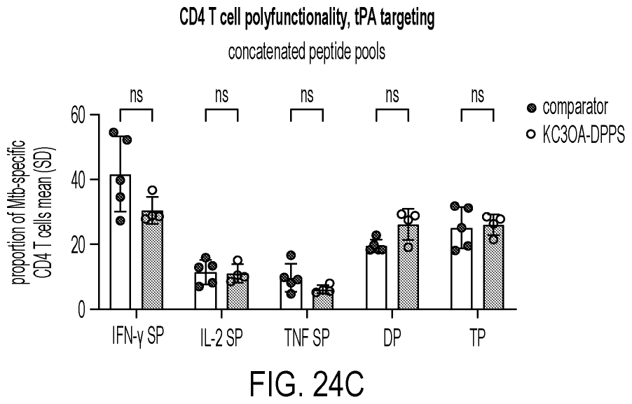

Cytokine polyfunctionality of vaccine-specific CD4 T cells is shaped by the LNP formulation and signal peptide/transmembrane domain (FIGS. 24A-24C). Data correspond to cumulative T cell responses shown in FIG. 22. Concatenated CD4 T cell responses across peptide pools were Boolean gated on cells that produced IFN-γ, IL-2 and TNF-α. SP, single producer; DP, double producer; TP, triple producer. All combinations of DP cells were combined for analysis. CD4 T cell responses were induced by the mRNA incorporating see/MITD targeting of nascent proteins to the endosomal compartment (FIG. 24A), the mRNA incorporating the LAMP-1 targeting of nascent proteins to the late endosomal/lysosomal compartment (FIG. 24B), or mRNA using the tPA signal peptide that directs proteins to be secreted (FIG. 24C).

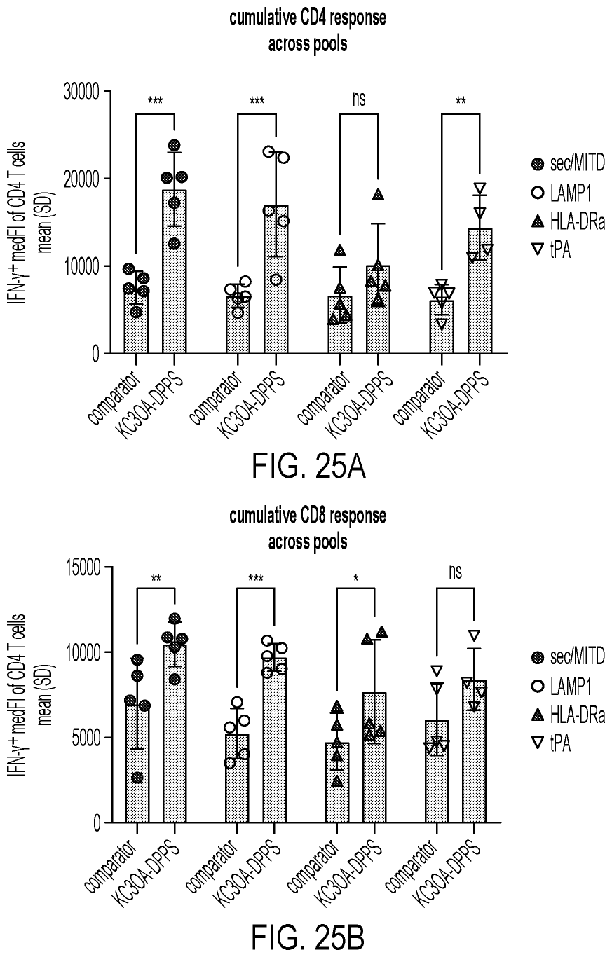

T cells induced by mRNA delivered with the KC3-OA/DPPS LNP formulation produce more per cell IFN-γ (FIGS. 25A-25B). Data correspond to cumulative T cell responses shown in FIG. 22. CD4 (FIG. 25A) and CD8 (FIG. 25B) Mtb-specific T cell responses were concatenated across peptide pools. The KC3-OA/DPPS LNP formulation induced superior per cell IFN-γ production than the comparator ALC-0315 LNP formulation.

### Example 35. Immunogenicity of Two HLA Class II-Directed Mtb mRNA Using KC3-OA/DPPS LNP Formulation and Comparison to BCG

The aim of this study was to explore the impact of the signal peptide on the immunogenicity of an HLA-II directed mRNA and compare this immunogenicity of BCG vaccine. The DPPS-NH₄, PEG-DMG and KC3-OA were kept constant at 5 mol %, 1.5 mol %, and 48 mol % respectively with an N/P ratio of 5.25 using mRNA encoding for the same concatenated Mtb antigens as in Example 34 with either sec/MITD (SEQ ID NOs. 37 and 38) or LAMP-1 signal peptides (SEQ ID NOs. 39 and 40). All mRNAs used N1-methylpseudouridine (N1MeU). The characterization of these mRNA-LNPs is shown below in Table 56. The DSPC and cholesterol were also kept constant at 5 mol % and 40.5 mol %, respectively in the KC3-OA LNP formulations. The two investigational mRNA LNPs formulations were compared to standard BCG s.c. vaccination.

TABLE 56
Physicochemical properties and characterization of 5 mol % DPPS-targeted
KC3-OA LNPs. Particle size refers to the Z-Average particle size. Post-
F/T refers to samples that were measured after a freeze/thaw event, and
Zeta refers to zeta potential measurements at either pH 5 or pH 7 in
units of mV. mRNA encapsulation efficiency is referred to as (E.E, %).

| | | Particle | Particle Size | | Zeta, | Zeta, |
|---|---|---|---|---|---|---|
| ICL | mRNA | size (nm) | (nm) Post-F/T | E.E, % | pH 5 | pH 7 |
| KC3-OA | SEQ ID | 105.2 | 108.7 | 91.2 ± 2.8 | 10.4 | −1.49 |
| | NOs. 37 and | | | | | |
| | 38 | | | | | |
| KC3-OA | SEQ ID | 107.1 | 108.4 | 90.1 ± 1.5 | 10.1 | −2.29 |
| | NOs. 39 and | | | | | |
| | 40 | | | | | |

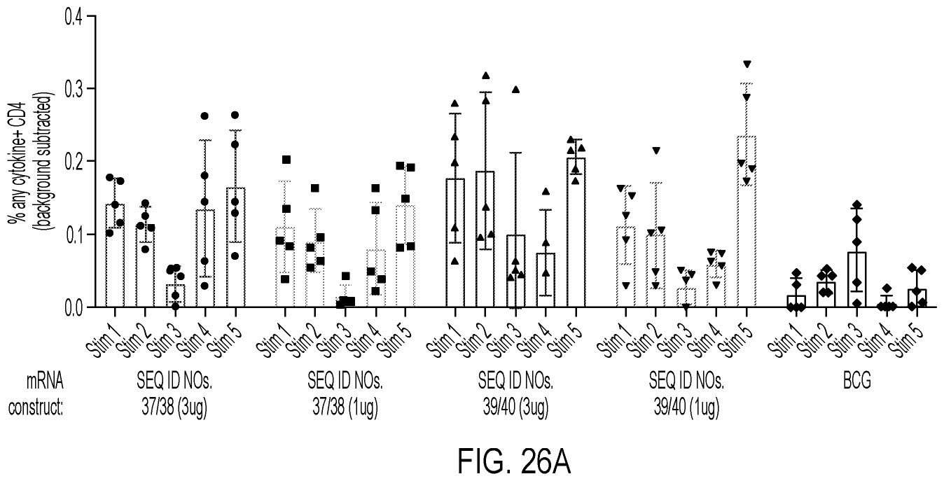

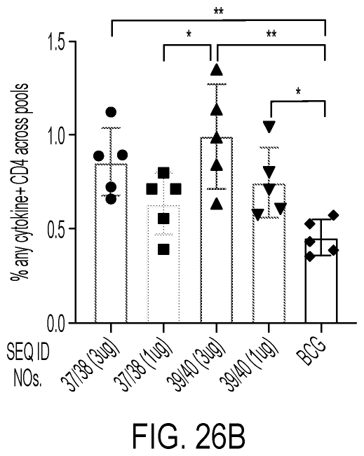

mRNA vaccination using both KC3-OA/DPPS LNPs induced a larger CD4 T cell response than BCG to the antigens encoded by the mRNA in the KC3-OA/DPPS LNP vaccines (FIGS. 26A-26B). CB6F1 mice were immunized i.m. with 3 μg or 1 μg of each mRNA. A positive control group was immunized s.c. with BCG. 28 days post immunization, all groups except the BCG group were given a homologous boost. Spleens were harvested on day 7 post-boost and splenocytes were stimulated with overlapping peptide pools (15mers overlapping by 11 amino acids). Mtb-specific CD4 T cells were defined as cells expressing IFN-γ, TNF-α, IL-2, IL-17a or combinations thereof. CD4 responses to individual Mtb antigens were evaluated in FIG. 26A. Stim 1: EsxH/TB10.4 and Ag85B peptide pools, Stim 2: Mtb39a peptide pool, Stim 3: EsxW and Esx V peptide pools, Stim 4: EsxB/CFP10 and EsxA/ESAT-6 peptide pools, Stim 5: peptide pool of ten C-terminal tandem 15mer minimal epitopes. Cumulative CD4 T cell response from all peptide stimulations (Sim 1+Stim 2+Stim 3+Stim 4+Stim 5-background) are shown in FIG. 26B.

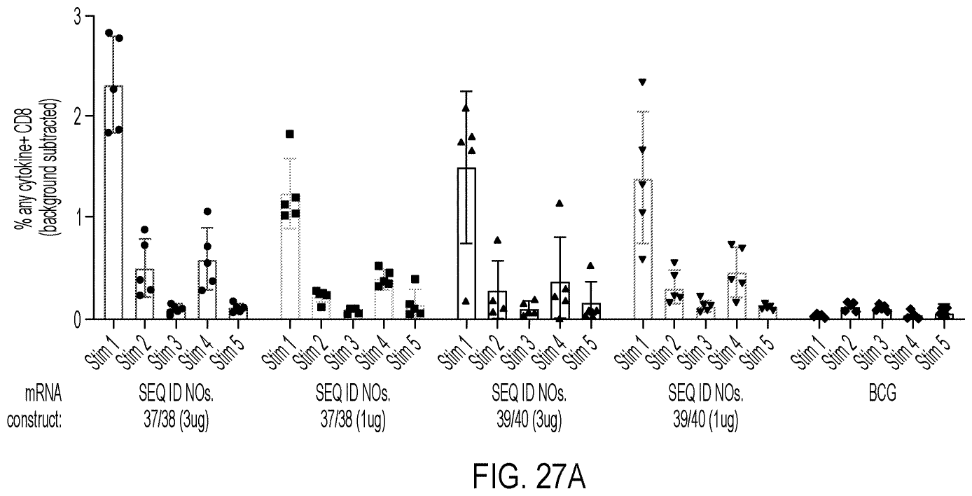

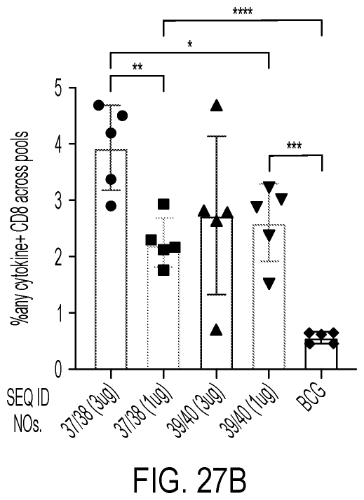

mRNA vaccination induced a larger CD8 T cell response than BCG to the antigens encoded by either of the two targeted mRNA vaccine constructs (FIGS. 27A-27B). Data correspond to the same study and animals shown in FIGS. 26A-26B. Mtb-specific CD8 T cells expressing IFN-γ, TNF-α, IL-2, IL-17a or combinations thereof were cumulatively added for each peptide pool stimulation. CD8 responses to individual Mtb antigens were defined as cells expressing IFN-Y, TNF-α, IL-2, IL-17a or combinations thereof. Stim 1: EsxH/TB10.4 and Ag85B peptide pools, Stim 2: Mtb39a peptide pool, Stim 3: EsxW and EsxV peptide pools, Stim 4: EsxB/CFP10 and EsxA/ESAT-6 peptide pools, Stim 5: peptide pool of ten C-terminal tandem 15mer minimal epitopes (FIG. 27A). Cumulative CD8 T cell response from all peptide stimulations (Sim 1+Stim 2+Stim 3+Stim 4+Stim 5-background) are shown in FIG. 27B. In conclusion, delivery of these mRNAs formulated with KC3-OA/DPPS LNPs generates focused T cell responses to antigens known to be protective in animal models that are quantitatively larger than those induced by BCG vaccination.

### Example 36. Immunogenicity of Three Unique HLA Class I-Directed Mtb mRNAs Using KC3-OA/DPPS LNP Formulation

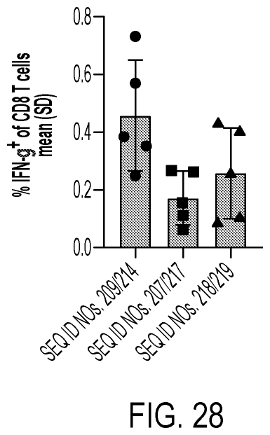

The purpose of this study was to examine CD8 T cell responses to antigen cassettes consisting of putative human HLA class I-restricted epitopes arranged in a string-on-bead format. An additional aim was to also examine the use of two different strategies for targeting nascent proteins to the proteasome and MHC class I presentation pathway. Using the KC3-OA/DPPS LNP formulations as in Example 35, three different mRNAs were encapsulated with the final mRNA-LNP characteristics shown in Table 57. All mRNAs used N1-methylpseudouridine (NIMeU). The first mRNA encoded for a mixture of Mtb antigens expressed only by Mtb or by both Mtb and BCG, and a nucleotide sequence encoding for a single ubiquitin was included at the 5′ end of the ORF (SEQ ID NOs. 209 and 214, Table 50). The second mRNA encoded for antigens expressed only by Mtb and not BCG with a single ubiquitin encoded at the 5′ end (SEQ ID NOs. 207 and 217, Table 48). The third mRNA encoded for the same “Mtb-only” antigens as the second mRNA, but an IkBalpha destruction motif was incorporated into the 5′ end of the ORF (SEQ ID NOs. 218 and 219, Table 48). CB6F1 mice were immunized i.m. with 1 μg mRNA. 4 weeks post prime, mice were boosted and spleens were harvested 7 days later. Splenocytes were stimulated with 15mer peptide pools overlapping by 11 amino acids and cells were stained intracellularly for IFN-g, TNF-a and IL-2, CD8 T cell responses specific to antigens encoded by each mRNA were detected (FIG. 28). However they were small in magnitude, and this is likely because the epitopes were chosen based on predicted affinity to human class I molecules and not epitopes restricted to murine H-2b and H-2d class I molecules. The second and third groups that shared the same polypeptide sequence were quantitatively similar, indicating that both the ubiquitin or IkBalpha destruction motif are viable methods for proteasomal targeting and MHC class I presentation.

SEQ ID NO. 214. Codon-optimized mRNA forward open reading frame sequence corresponding
to the polypeptide in SEQ ID NO. 209. The sequence contains 3′ and 5′ HBB UTRs (SEQ ID
NO. 32). N1-methylpseudouridine is substituted for all uridines (U) during mRNA synthesis.
ACAUUUGCUUCUGACACAACUGUGUUCACUAGCAACCUCAAACAGCCACCAUGCAAAUCUUCGU
CAAGACCCUCACCGGAAAGACCAUUACACUGGAAGUCGAGCCCUCCGAUACUAUCGAGAACGUG
AAGGCCAAGAUCCAGGACAAGGAGGGCAUUCCCCCGGACCAGCAGCGCCUGAUCUUCGCGGGGA
AGCAGCUGGAAGAUGGACGGACCCUGUCCGACUACAACAUCCAGAAGGAAUCCACUCUUCACCU
CGUGCUGCGCCUGAGAGGAGCGUUCUACGUGUGGGACUUUGCCGAGAAGUUCAAGGAGGACGUG
AUCAACGACUUCGUGUCAUCCUACGCCGUGGCCGACCCUAUGGGAGCCGCCUUCGACUACGCCG
CCGAAGUCCUGCGGGUUCCGCCCCGCCCCUAUGUCAUGCCGCACUCCCCCGCCGCCGGAGCGGC
GAUUCCUGCGGUGGUGCCCGAACUGACGCCGGUGGCAGCCGCCCCACCCAUGCUGGCCGUGGCA
AUCGAGAAGCCCGUAGCCCCAAGCGUCAUGGACUUCCCGGAUUCGGGAACACACAGCUGGGAAA
GAGCCGUCCCCGGACGGGUGGUGGCCCUGAGUUUGCUUGAUGCCCACAUUCCCCAACUCGUGCG
CGUGCUGGAUGCCCUGACCGACAAACUCGCCGAUACGGACCCCCUGCCUGUCGUGUUUUGGGAG
AGAAACGACCCGACACAGCAGAUUGUGCGGGCCGCCACUACCAUGGCUACUUACGUGUCCUCCC
CCGAUGUGCUCACCACCUAUAUUGCCGCGGCCGACGAAGUGUCGACCCAGGUCGCCAUCGAUGA
ACUGAAAACCAAUAGCUCACUGCUGACCUCCAUCCUCACUUACUUCGCCGCCCCCGUGGCACUG
GCAGCCUACCCUAUCACUGGAAAGCUGGCUGCGGCUGUGGAUAUCAGAGAGACUUUCAGGAUGG
CAGUGGUCGGAGGCGCCCUGGCGUACCUGGUGGUCAAAACGCUGAUCAACGCCACCCAGCUGCU
CAAGCUCUUGGCGAAACUGGCCACCAUUGCCACUUUCGAGAUGCGGUACCUCCAGUCACUUCCC
GCCCACUUGAGGCCGGGCCUGGAUGAACUGGCUACCAUCGACGUGGCCAUCCCCAAGCUGAGGG
GCGGUACUCAUCCCACUACUACCUACAAGGCGUUUGAUUGGGAUCAGGCAUACCGCAAGCCCAU
UACCUACAUCAUGUAUAACUACCCUGCGAUGCUCGGUUACUACCAGUCCGGCCUGUCGAUUGUG
AUGGCCGCGCGGGCACUGCCAUUAACCUCACUGACCUCCGCCGCUGAGCGGGGACCGGGGCAGA
UGCUCGUGGCCGAGCUGAUUGGCGACGCCCUGUUCGUGCAACUGCACGCGCCGCUUCGGGGCGU
GGCCCGCGGACAGACUCUCGUCCUUUACGUGGAAAUGGCGGCUCAGCAGCACGUGGUCAUCGAG
GCCUACACCGCUGCGGUGGAACUCAUGCCUACUCAAGUGGAGCUGGCGGACACUAGAGUCGCCA
CCCCAUCGGGUCUGGACGGUCCUGGGAUGAGCACGUCCGCAUACGACAUCGGCCUGUUCUACCG
CUAUGCCUGGCAAAACCCUGUGUUCGCGUUCCACGACCAGUUCGUGCAGGCUUUGAGAGCCAAG
CUGCUGUCCUCGAUCCUGACCUACCACUCGGAACUGCGAGCCUUGGUGGAGGUGUACCCUAUUU
CCCCUCCUGGUUUCCCGUCCCUGCCUUACGCCAUGACCCUGCCGCCACCAGUGGUGGCCGCCAA
CAGGAUACAGUUGCUGUCCCGCUUCAUGACUGAUCCCCACGCCAUGAGGGGGUUCGCAAUUCCG
AUUGGCCAGGCCAUGGCUAUGUACGCCGGCCCUGGAAGCGCAUCCCUCGCCACCUACGACAAGG
GAAUCCUGACCGUGGGAUACACCGACGACGCCGGCCAGACCUUCGUGCAAGAGCUCGACGAAAU
CUCCACCAACAUCCAAGCCGCCGAGGCCUUCGUCGCCGCCUACAUGCCUUUCCUGAUCCUGGCU
CCGCAAAUCAACUUCUUCUACAUCCUUGCCGCAUACCACCCACAACAGUUCAUCUACGCCGGGU
CGUUGAGCGCCUUGCUGGAACUUGAGAACGACAAUCAGCUGCUCUACGACUACGUGUACCCUGG
ACACGCCAUUUGGUGGCUCGCACGCGGGCUCGAGUUUUUCCAAGACGGGGAACAGUUUGGCGAA
CUGCUUUUUACUAACCCGACCGGCGCCUUCACCCUGGCCAUCUACCAUCCGCAACAGUUCGUGU
ACAUCGCCGUGAUGCCGCCGGCGCCAGGGAUGGUGUUCUCCCGGCCUGGGCUGCCGGUGGAAUA
CCUCCAAGUGCCGUCACCAAGCAUGGGAGCGGCAUUCCAGGGCGCUCACGCUCGGUUCGGAAGG
GCCAGCGGAGUGCUGCCGCGCUACGGUCUGCCUCCGCCAUGGCAGCCUGCCCUGCGGCAUCCGC
GCUCCCUCUUCCCUGAAUUCUCCGAACUAUUCGCCGCUUUCCCCUCGUUCGCCGGCCUGCGGCC
GACCUUCGACACCCGGCUGAUGCGGUCCAGAAUCGAUGGAACCCAUCAGACCCUGGUGGCAGCG
UGGGAUAAGGUCAUGAACCUCACCUACAUCCCCGUCGUGGGUCACGCGUUGUCCAUUCGCAUGU
GGAACCAGGCUGCGCUGGCUAUGGAAGUGUACCAGGCAGAAACCGCAGUGAACACUCUGUUUUC
ACAAGCGACCGACCCCCUGUCCCUGCUGAUCGAAACCGUGACUCAGGCCCUGCAAGCCCUUACC
AUCCCGUCCUUCAUUCCUGAGGACUUCACCUUUUUGGUGCACGCCGCCUCCACCCGCCAAGCGC
UGAGAUCCCAGUUCAACGACACCCUGAAUGUCUAUCUCCUCCCUCUGCCGAACAUUCCCCUGCU
UGAAUACGCUGCCCGGUUCAUCACCCCCGUGCACCCCGGGUAUACUGCCACCUUCCUGGAGACU
CCGAGCCAGUUCUUCCCGUUUACCGGCCUGAAUUCCCUGACCCUCCUCGACGCCCACAUCCCAC
AACUGAUCAGCGCGCAGGCCGCCGCCAUUCAUGAGAUGUUUGCGUUCCAAUACGACGGCGUGAA
CGACUUCCCGAAGUACCCCCUGAACGUGUUCGCCACCGCGAACGCUAUCGCCGGAAUCCUCUUC
CUCCAUUCGGGACUCAUUGCGCUGCCUCAUACUUCUCCGCCCCCAUUGAUCCUGAAGUCGAUGG
UCCGCGACGGACAGCUGACUAUCAAGGGUUCUGUGACUCCGGCAGUCAGCCAGUUUAACGCUAG
AACCGCUGACGGCAUUAACUACCGGGUGGCCUUCCCUCCGGAAGUGCACAGCGCCAUGCUGAAG
GAGCUGGAUGAGGCCGUGGAGGCCUUCUGAUAAUAGGCUCGCUUUCUUGCUGUCCAAUUUCUAU
UAAAGGUUCCUUUGUUCCCUAAGUCCAACUACUAAACUGGGGGAUAUUAUGAAGGGCCUUGAGC
AUCUGGAUUCUGCCUAAUAAAAAACAUUUAUUUUCAUUGCAA
SEQ ID NO. 217. Codon-optimized mRNA forward open reading frame sequence corresponding
to the polypeptide in SEQ ID NO. 207. The sequence contains 3′ and 5′ HBB UTRs (SEQ ID
NO. 32). N1-methylpseudouridine is substituted for all uridines (U) during mRNA synthesis.
ACAUUUGCUUCUGACACAACUGUGUUCACUAGCAACCUCAAACAGCCACCAUGCAAAUCUUCGU
CAAGACCCUCACCGGCAAAACCAUUACCCUGGAAGUCGAACCUAGCGACACCAUCGAGAACGUG
AAGGCCAAGAUCCAGGAUAAGGAGGGGAUUCCGCCGGACCAGCAGAGGCUUAUCUUUGCCGGGA
AGCAGCUCGAGGACGGAAGGACCCUGUCCGACUACAACAUCCAGAAGGAGAGCACACUGCAUUU
GGUGCUCCGCCUGCGGGGCGCCUGGAGACAUUGGGUGCAUGCCCUGACACGCAUUAACCUGGGC
CUGUCCCCCGAUGAGAAAUACGAACUGGACCUCCAUGCGCGCGUGCGCCGGAACCCUCGGGGAU
CGUACCAGAUCGCCGUCGUGGGGCUGCAGCUUCCACCGGCGGCCACUCAGACCCUCCCUGCCGC
ACCUGUGACUACCGCCGCAAUGGCUGACCCCGCCGCCGACUUGAUUGCGCCCAAAACUUACUGC
GAAGAACUCAAGGGGACCGACACGGGACAGGCUUGCCAGAUUCAGAUGAGUGACCCUGCCUACA
ACAUUAACAUCUCCCUUCCUUCUUACUAUCCGGAUCAGAAGUCACUCGAGAACUACAUCGCCCA
AACCCGGGAUAAGUUUCUGUCCGCCGCCACUUCAUCCACCCCUCGCGAAGCCCCCUACGAGCUG
AAUAUCACCUCGGCGACCUACCAGUCCGCUAUCCCGCCUCGGGGGACUCAGGCUGUGGUGCUCA
AAGUGUACCAGAACGCCGGCGGUACCCACCCGACCACUACGUACAAGGCCUUCGAUUGGGACCA
GGCCUACCGGAAGCCAAUCACCUAUGACACGCUGUGGCAGGCCGACACCGACCCGCUUCCUGUG
GUGUUCCCGAUCGUGCAGGGCGAAUUGUCCAAGCAAACUGGCCAACAAGUGUCCAUAGCGCCGA
ACGCCGGGUUGGACCCCGUGAACUACCAGAACUUCGCCGUGACCAACGACGGAGUGAUUUUCUU
CUUCAACCCUGGAGAACUGCUGCCGGAGGCCGCCGGCCCGACCCAAGUGCUCGUGCCGAGAUCG
GCCAUUGAUUCCAUGCUCGCCGCGGAAACCGAUACGGAGCAGGAGAUUGCCCUGUUCACUGUGA
CAAACCCACCGAGGACUGUGUCCGUGUCCACUCUGAUGGACGGACGGAUCGACCAUGUGGAGCU
AAGCGCCAGGGUGGCCUGGAUGAGCGAAUCACAGCUCGCAUCCGAGAUUCUGGUCAUCGCUGAC
CUGGCCAGACAGAAGGCCCAGUCCGCACAGUACGCAUUCAUCCUGGACCGCAUGUCGCAACAAG
UGGAUGCCGACGAGCACAGGGUGGCGUUGCUGGGAUGGACCGCAGACCCUAUCAUCGGCGUUCA
AGUCCGCGCAGACCGCAUCCUCGCGCUGGCGGUGGCCGCCUCGAACAACCCGGAACUGACCACC
CUGACCGCCGCUCUGUCCGGCCAACUGAACCCACAAGUCAACCUCGUGGACACCUUGAAUUCCG
GACAGUACACCGUGUUCGCCCCGACCAACGCUGCGUUCUCCAAGCUGCCGGCCUCAACUAUCGA
CGAACUCAAGACAAACUCCAGCCUGCUGACCUCGAUCCUCACCUACAACCACAUUAUGCCGGGA
GAACCUAACGUGGCCGUCAAAGACCUUGUGCGCCACUUCGAGCAACAGGUCCAGCCCGGCCGAG
UGGUGGUCAUGCCCUGGGAUCGGCACAUCGCCGCCGGCACUGAAAUUUCGCUGGAUCUCCUGGA
CCCAAUCUACAAGCGGAAGGUCCUGGAGCUGGCAGCGGCGCUCCAAGCCGAAACCGCCGUGAAC
ACUCUGUUCGAGAAGCUGGAGCCUAUGGCUGCCGCCUCCAAGCCUCCCACUCCUCCCAUGGUGC
ACGCAGCUAGCACCAGACAGGCUCUGAGAGCCCACUUCGCCGACGGUUGGAACACCUUCAACCU
GGCCGAAGCUGAAGUGUUCGCGACUAGAUACUCCGCGGCAAAGAAUGCAGCUCAGCAGCUGGUG
CUGUCAGCCGAUAACAUGCGGGAGUACCUGGCCGCGGGAGCCAAGGAAAGACAGAGGCUGGCAA
CCUCGCUGAGAAACGCCGCGAAGCGCCUGUAUGCUGAAAACCCCUCAGCCCGCGACCAGAUCCU
GCCGGUGUACGCCGAAUACCAGCAGCGGAGCGAAAAGGUCCUCACCGAGUACAACAACAAGGCG
GCGCUGGAUCAGCAGCGGCAGUGGAUUUUGCACAUGGCUAAGUUAUCCGCUGCCAUGGCCAAGC
AGGCCCAAUACGUGGCCCAGCUUCACGUCUGGGCUCGGCGCGAACACCCUACCUACGAAGGGGU
GCACACCGCCAACGCUACGGUGUACAUGAUCGAUACCGUGCUGAUGGCCUCGAAGGGUACUACC
ACCAAGAAGUACAUGGCAGCCGACUACGACAAGCUGUUCCGCGCCACUAUCGCGGACGUGCUGG
CCGAGAAGGAGCUGAGCCAUUACAACGACAUCCGGGCCCACACCUCUGUGAACGCCGUGAAUCU
CGAGGUGCUGCCCGCUCCGGAGUAUUCCUCGGCGCAAAGAGCCUUGAGCGAUGCCGAUUGGCAC
UUCAUCGCGGACCCCGCCUCCCGGUUCUACAAUCUGGUACUGGCCUUUGUGCGGCCCGUCGCUG
UCGAUCUGACUUACAUCCCCGUGGUCGGACACGCCCUGAGCGCCAGCCAGUUCAACGACACUCU
GAACGUGUACCUGACUGCCCACAACGCCCUUGGAAGCAGCCUGCACACUGCCGGAGUGGACCUC
GCCAAGUUCUUUGAUCCCCUUACCCGCGGGGUGUUGACCAGGACUCCGCUCAUGUCCCAACUGA
UCUUCAUCAUCGACCCCACCAUUAGCGCCAUUGACGGCCUCUACGACCUUCUCGGUAUUGGUAU
CCCAAAUCAGGGUGGCAUCCUGUACUCCUCACUGGAAUACUUCGAAAAGGCCCUGGAGGAGCUC
GCCGCCGAACAAGUCGGUGGCCAGUCGCAGCUGGGAAAGCUGAACCCAGAUGUGAACCUGGUGG
ACACCCUGAACGGAGGAGAGUACACUGUGUUUGCCCCAACCAACGCCGCGUUCGACAAGCUCCC
CGCGGCAACCAUCGACCAGCUGAAAACUGAUGCCAAGCUGCUCUCCUCCAUUCUGACCUACCAU
GUCAUCGCGGGACAAGCAUCCCCCUCCCGAAUCGACGGGACUCACCAGACCCUUCGGCGGACUG
CGCCAGCACCCCCGUGGGCGAAGAUGAUGGCAGUGGUCGGAGGCGCCCUGGCUUACCUGGUGGU
CAAGACCUUGAUCAAUGCCACCCAGCUGCUCAAGCUGCUGGCCAAGCUGGGAGUGUCCACCGCG
AACGCCACCGUGUAUAUGAUUGAUUCGGUGCUGAUGCAGCCUUUCUUCGACCCGAGCGCGUCAU
UCCCCUGAUAAUAGGCUCGCUUUCUUGCUGUCCAAUUUCUAUUAAAGGUUCCUUUGUUCCCUAA
GUCCAACUACUAAACUGGGGGAUAUUAUGAAGGGCCUUGAGCAUCUGGAUUCUGCCUAAUAAAA
AACAUUUAUUUUCAUUGCAA
SEQ ID NO. 218. Example of an amino acid sequence for an HLA-I “Mtb-only” antigen with an
N-terminal IkBalpha destruction motif peptide (SEQ ID NO. 206). The IkBalpha motif is
underlined.
MERLLDDRHDSGLDSMKDEEWRHWVHALTRINLGLSPDEKYELDLHARVRRNPRGSYQIAVVGL
QLPPAATQTLPAAPVTTAAMADPAADLIAPKTYCEELKGTDTGOACQIQMSDPAYNINISLPSY
YPDQKSLENYIAQTRDKFLSAATSSTPREAPYELNITSATYQSAIPPRGTQAVVLKVYQNAGGT
HPTTTYKAFDWDQAYRKPITYDTLWQADTDPLPVVFPIVQGELSKQTGQQVSIAPNAGLDPVNY
QNFAVTNDGVIFFFNPGELLPEAAGPTQVLVPRSAIDSMLAAETDTEQEIALFTVINPPRTVSV
STLMDGRIDHVELSARVAWMSESQLASEILVIADLARQKAQSAQYAFILDRMSQQVDADEHRVA
LLGWTADPIIGVQVRADRILALAVAASNNPELTTLTAALSGQLNPQVNLVDTLNSGQYTVFAPT
NAAFSKLPASTIDELKTNSSLLTSILTYNHIMPGEPNVAVKDLVRHFEQQVQPGRVVVMPWDRH
IAAGTEISLDLLDPIYKRKVLELAAALQAETAVNTLFEKLEPMAAASKPPTPPMVHAASTRQAL
RAHFADGWNTFNLAEAEVFATRYSAAKNAAQQLVLSADNMREYLAAGAKERQRLATSLRNAAKR
LYAENPSARDQILPVYAEYQQRSEKVLTEYNNKAALDQQRQWILHMAKLSAAMAKQAQYVAQLH
VWARREHPTYEGVHTANATVYMIDTVLMASKGTTTKKYMAADYDKLFRATIADVLAEKELSHYN
DIRAHTSVNAVNLEVLPAPEYSSAQRALSDADWHFIADPASRFYNLVLAFVRPVAVDLTYIPVV
GHALSASQFNDTLNVYLTAHNALGSSLHTAGVDLAKFFDPLTRGVLTRTPLMSQLIFIIDPTIS
AIDGLYDLLGIGIPNQGGILYSSLEYFEKALEELAAEQVGGQSQLGKLNPDVNLVDTLNGGEYT
VFAPTNAAFDKLPAATIDQLKTDAKLLSSILTYHVIAGQASPSRIDGTHQTLRRTAPAPPWAKM
MAVVGGALAYLVVKTLINATQLLKLLAKLGVSTANATVYMIDSVLMQPFFDPSASFP
SEQ ID NO. 219. Codon-optimized mRNA forward open reading frame sequence corresponding
to the polypeptide in SEQ ID NO. 218. The sequence contains 3′ and 5′ HBB UTRs (SEQ ID
NO. 32). N1-methylpseudouridine is substituted for all uridines (U) during mRNA synthesis.
ACAUUUGCUUCUGACACAACUGUGUUCACUAGCAACCUCAAACAGCCACCAUGGAAAGGCUUCU
GGACGAUAGGCAUGACUCCGGCUUGGAUAGCAUGAAGGACGAGGAGUGGAGACAUUGGGUGCAU
GCCCUGACACGCAUUAACCUGGGCCUGUCCCCCGAUGAGAAAUACGAACUGGACCUCCAUGCGC
GCGUGCGCCGGAACCCUCGGGGAUCGUACCAGAUCGCCGUCGUGGGGCUGCAGCUUCCACCGGC
GGCCACUCAGACCCUCCCUGCCGCACCUGUGACUACCGCCGCAAUGGCUGACCCCGCCGCCGAC
UUGAUUGCGCCCAAAACUUACUGCGAAGAACUCAAGGGGACCGACACGGGACAGGCUUGCCAGA
UUCAGAUGAGUGACCCUGCCUACAACAUUAACAUCUCCCUUCCUUCUUACUAUCCGGAUCAGAA
GUCACUCGAGAACUACAUCGCCCAAACCCGGGAUAAGUUUCUGUCCGCCGCCACUUCAUCCACC
CCUCGCGAAGCCCCCUACGAGCUGAAUAUCACCUCGGCGACCUACCAGUCCGCUAUCCCGCCUC
GGGGGACUCAGGCUGUGGUGCUCAAAGUGUACCAGAACGCCGGCGGUACCCACCCGACCACUAC
GUACAAGGCCUUCGAUUGGGACCAGGCCUACCGGAAGCCAAUCACCUAUGACACGCUGUGGCAG
GCCGACACCGACCCGCUUCCUGUGGUGUUCCCGAUCGUGCAGGGCGAAUUGUCCAAGCAAACUG
GCCAACAAGUGUCCAUAGCGCCGAACGCCGGGUUGGACCCCGUGAACUACCAGAACUUCGCCGU
GACCAACGACGGAGUGAUUUUCUUCUUCAACCCUGGAGAACUGCUGCCGGAGGCCGCCGGCCCG
ACCCAAGUGCUCGUGCCGAGAUCGGCCAUUGAUUCCAUGCUCGCCGCGGAAACCGAUACGGAGC
AGGAGAUUGCCCUGUUCACUGUGACAAACCCACCGAGGACUGUGUCCGUGUCCACUCUGAUGGA
CGGACGGAUCGACCAUGUGGAGCUAAGCGCCAGGGUGGCCUGGAUGAGCGAAUCACAGCUCGCA
UCCGAGAUUCUGGUCAUCGCUGACCUGGCCAGACAGAAGGCCCAGUCCGCACAGUACGCAUUCA
UCCUGGACCGCAUGUCGCAACAAGUGGAUGCCGACGAGCACAGGGUGGCGUUGCUGGGAUGGAC
CGCAGACCCUAUCAUCGGCGUUCAAGUCCGCGCAGACCGCAUCCUCGCGCUGGCGGUGGCCGCC
UCGAACAACCCGGAACUGACCACCCUGACCGCCGCUCUGUCCGGCCAACUGAACCCACAAGUCA
ACCUCGUGGACACCUUGAAUUCCGGACAGUACACCGUGUUCGCCCCGACCAACGCUGCGUUCUC
CAAGCUGCCGGCCUCAACUAUCGACGAACUCAAGACAAACUCCAGCCUGCUGACCUCGAUCCUC
ACCUACAACCACAUUAUGCCGGGAGAACCUAACGUGGCCGUCAAAGACCUUGUGCGCCACUUCG
AGCAACAGGUCCAGCCCGGCCGAGUGGUGGUCAUGCCCUGGGAUCGGCACAUCGCCGCCGGCAC
UGAAAUUUCGCUGGAUCUCCUGGACCCAAUCUACAAGCGGAAGGUCCUGGAGCUGGCAGCGGCG
CUCCAAGCCGAAACCGCCGUGAACACUCUGUUCGAGAAGCUGGAGCCUAUGGCUGCCGCCUCCA
AGCCUCCCACUCCUCCCAUGGUGCACGCAGCUAGCACCAGACAGGCUCUGAGAGCCCACUUCGC
CGACGGUUGGAACACCUUCAACCUGGCCGAAGCUGAAGUGUUCGCGACUAGAUACUCCGCGGCA
AAGAAUGCAGCUCAGCAGCUGGUGCUGUCAGCCGAUAACAUGCGGGAGUACCUGGCCGCGGGAG
CCAAGGAAAGACAGAGGCUGGCAACCUCGCUGAGAAACGCCGCGAAGCGCCUGUAUGCUGAAAA
CCCCUCAGCCCGCGACCAGAUCCUGCCGGUGUACGCCGAAUACCAGCAGCGGAGCGAAAAGGUC
CUCACCGAGUACAACAACAAGGCGGCGCUGGAUCAGCAGCGGCAGUGGAUUUUGCACAUGGCUA
AGUUAUCCGCUGCCAUGGCCAAGCAGGCCCAAUACGUGGCCCAGCUUCACGUCUGGGCUCGGCG
CGAACACCCUACCUACGAAGGGGUGCACACCGCCAACGCUACGGUGUACAUGAUCGAUACCGUG
CUGAUGGCCUCGAAGGGUACUACCACCAAGAAGUACAUGGCAGCCGACUACGACAAGCUGUUCC
GCGCCACUAUCGCGGACGUGCUGGCCGAGAAGGAGCUGAGCCAUUACAACGACAUCCGGGCCCA
CACCUCUGUGAACGCCGUGAAUCUCGAGGUGCUGCCCGCUCCGGAGUAUUCCUCGGCGCAAAGA
GCCUUGAGCGAUGCCGAUUGGCACUUCAUCGCGGACCCCGCCUCCCGGUUCUACAAUCUGGUAC
UGGCCUUUGUGCGGCCCGUCGCUGUCGAUCUGACUUACAUCCCCGUGGUCGGACACGCCCUGAG
CGCCAGCCAGUUCAACGACACUCUGAACGUGUACCUGACUGCCCACAACGCCCUUGGAAGCAGC
CUGCACACUGCCGGAGUGGACCUCGCCAAGUUCUUUGAUCCCCUUACCCGCGGGGUGUUGACCA
GGACUCCGCUCAUGUCCCAACUGAUCUUCAUCAUCGACCCCACCAUUAGCGCCAUUGACGGCCU
CUACGACCUUCUCGGUAUUGGUAUCCCAAAUCAGGGUGGCAUCCUGUACUCCUCACUGGAAUAC
UUCGAAAAGGCCCUGGAGGAGCUCGCCGCCGAACAAGUCGGUGGCCAGUCGCAGCUGGGAAAGC
UGAACCCAGAUGUGAACCUGGUGGACACCCUGAACGGAGGAGAGUACACUGUGUUUGCCCCAAC
CAACGCCGCGUUCGACAAGCUCCCCGCGGCAACCAUCGACCAGCUGAAAACUGAUGCCAAGCUG
CUCUCCUCCAUUCUGACCUACCAUGUCAUCGCGGGACAAGCAUCCCCCUCCCGAAUCGACGGGA
CUCACCAGACCCUUCGGCGGACUGCGCCAGCACCCCCGUGGGCGAAGAUGAUGGCAGUGGUCGG
AGGCGCCCUGGCUUACCUGGUGGUCAAGACCUUGAUCAAUGCCACCCAGCUGCUCAAGCUGCUG
GCCAAGCUGGGAGUGUCCACCGCGAACGCCACCGUGUAUAUGAUUGAUUCGGUGCUGAUGCAGC
CUUUCUUCGACCCGAGCGCGUCAUUCCCCUGAUAAUAGGCUCGCUUUCUUGCUGUCCAAUUUCU
AUUAAAGGUUCCUUUGUUCCCUAAGUCCAACUACUAAACUGGGGGAUAUUAUGAAGGGCCUUGA
GCAUCUGGAUUCUGCCUAAUAAAAAACAUUUAUUUUCAUUGCAA

TABLE 57
Physicochemical properties and characterization of 5 mol % DPPS-targeted
KC3-OA LNPs. Particle size refers to the Z-Average particle size. Post-
F/T refers to samples that were measured after a freeze/thaw event, and
Zeta refers to zeta potential measurements at either pH 5 or pH 7 in
units of mV. mRNA encapsulation efficiency is referred to as (E.E, %).

| | | | Particle | | | |
|---|---|---|---|---|---|---|
| | | Particle | Size (nm) | | Zeta | Zeta pH |
| ICL | mRNA | Size (nm) | Post-F/T | E.E, % | pH 5 | 7 |
| KC3-OA | SEQ ID NOs. 209 | 110.3 | 112.4 | 87.1 ± 5.9 | 7.5 | −3.5 |
| | and 214 | | | | | |
| KC3-OA | SEQ ID NOs. 207 | 111.4 | 111.5 | 92.4 ± 4.7 | 17.1 | −2.0 |
| | and 217 | | | | | |
| KC3-OA | SEQ ID NOs. 218 | 112.8 | 111.2 | 90.8 ± 3.7 | 17.2 | −1.3 |
| | and 219 | | | | | |

### Example 37. Time Dependence of Immunogenicity of HLA Class I- and HLA Class II-Directed Mtb mRNA Using a KC3-OA/DPPS LNP Formulation

The aim of this study was to explore the kinetics of vaccine-specific T cell responses for two Mtb mRNA vaccine constructs using KC3-OA/DPPS LNPs. The first was an HLA-II directed mRNA using a sec/MITD signal peptide (SEQ ID NOs. 37 and 38) and the second was an HLA-I directed mRNA where the antigenic sequence was preceded by a nucleotide sequence encoding ubiquitin with a G76A mutation (SEQ ID NOs. 207 and 217). All mRNAs used N1-methylpseudouridine (NIMeU). The DPPS-NH₄, PEG-DMG and KC3-OA were kept constant at 5 mol %, 1.5 mol %, and 48 mol % respectively with an N/P ratio of 5.25. The DSPC and cholesterol were also kept constant at 5 mol % and 40.5 mol %, respectively in the KC3OA LNP formulations. The characteristics of the mRNA-LNPs are shown below in Table 58.

LNPs were prepared as described in Example 9, characterized for particle size and zeta potential as described in Example 10, mRNA encapsulation efficiency and mRNA concentration as described in Example 10 and evaluated for antigen-specific CD4- and CD8-T cell responses in the spleens of CB6F1 mice over time, from day 5 to day 12 post four week boost at dose of 3 μg mRNA.

TABLE 58
Physicochemical properties and characterization of 5 mol % DPPS-targeted
KC3-OA LNPs. Particle size refers to the Z-Average particle size. Post-
F/T refers to samples that were measured after a freeze/thaw event, and
Zeta refers to zeta potential measurements at either pH 5 or pH 7 in
units of mV. mRNA encapsulation efficiency is referred to as (E.E, %).

| | | | Particle | | | |
|---|---|---|---|---|---|---|
| | | Particle | Size (nm) | | Zeta | Zeta pH |
| ICL | mRNA | Size (nm) | Post-F/T | E.E, % | pH 5 | 7 |
| KC3-OA | SEQ ID NOs. 37 and | 115.4 | 114.1 | 87.1 ± 5.9 | 15.55 | −0.08 |
| | 38 | | | | | |
| KC3-OA | SEQ ID NOs. 207 | 114.7 | 113.5 | 92.4 ± 4.7 | 20.3 | −0.22 |
| | and 217 | | | | | |

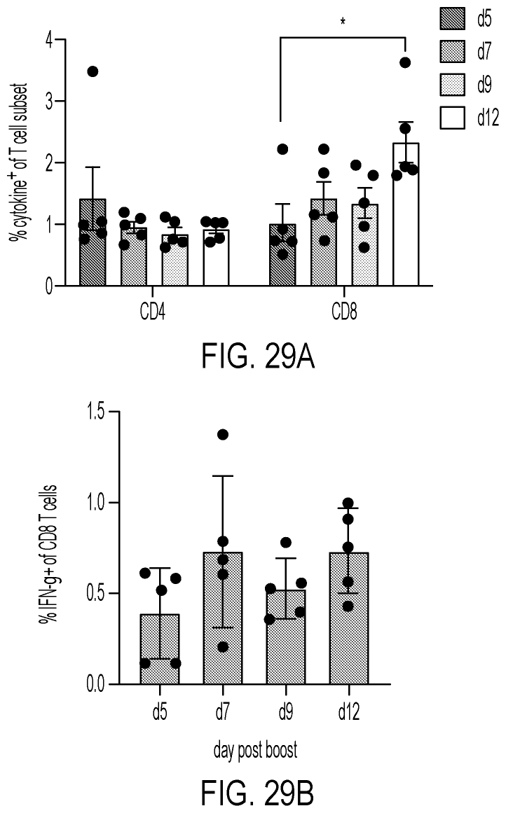

To understand the kinetics of vaccine-specific T cell responses using both HLA class I- and HLA class II-directed mRNA vaccines employing KC3-OA/DPPS LNPs, CB6F1 mice were immunized with 3 μg mRNA encapsulated in KC3-OA/DPPS LNPs and boosted 4 weeks later (FIGS. 29A-29B). Spleens were harvested either 5, 7, 9 or 12 days later and cells were stimulated with overlapping peptide pools covering the antigenic polypeptides. Antigen-specific CD4 and CD8 T cells were defined as cells expressing IFN-γ, TNF-α, IL-2, or combinations thereof. The cumulative CD4 and CD8 T cell responses to all peptide pools are shown for mice immunized with the HLA class II-directed mRNA (FIG. 29A). The cumulative CD8 T cell responses to all peptide pools are shown for mice immunized with the HLA class I-directed mRNA (FIG. 29B). The kinetics for the HLA-II directed LNP remained relatively constant over the 5-12-day period for CD4 responses, while the CD8 responses appeared to continue to increase between days 5 and 12. For the HLA-I directed LNP, the CD8 response similarly increased from day 5 to day 12. These kinetics demonstrate that we are capturing the peak, or near peak, magnitude of vaccine-specific CD4 and CD8 T cell responses at the standard time of day 7 post boost used in Examples 34-36. This is important to comparing the magnitude of T cell responses elicited by these mRNA-LNP vaccines to other published TB vaccine candidates using the mouse model.

### Example 38. Comparison of Immunogenicity of HLA Class II-Directed Mtb mRNA Containing Unique Coding Sequences Using a KC3-OA/DPPS LNP Formulation and Impact of Twenty Mol % Phospholipid Composition

The aim of this study was to explore the immunogenicity of HLA-II directed mRNAs that differed in some of the encoded Mtb antigens using KC3-OA/DPPS LNPs, and also to compare a 5 and 10 mol % DSPC version of this formulation. Multiple changes were made to the 1^{st} generation HLA-II mRNA to incorporate Mtb antigens predicted to be protective. First, EsxH/TB10.4 was removed because multiple lines of evidence indicate that, while TB10.4 generates immunodominant T cell responses these are not protective, infected macrophages do not efficiently process and present TB10.4-derived epitopes to T cells (PLoS Pathog. 2018 May 21; 14(5), PMID 29782535; PLoS Pathog. 2020 Oct. 19; 16(10), PLoS Pathog. 2020 Oct. 19; 16(10), PMID 33075106; Gene Ther., 2012 May; 19(5):570-5, PMID 21956689). Thus, TB10.4 could be a “decoy” antigen that promotes non-protective T cell responses at the expense of other protective T cell specificities. In a study comparing TCR cluster specificities between LTBI controllers versus those that progressed to active disease, CD4 T cells specific to Rv1195/PE13 were elevated in controllers (Nat Med 2023 January; 29(1):258-269, PMID 36604540), suggesting that PE13-specific T cells may be protective. Thus, in place of EsxH/TB10.4, the entirety of PE13 was added to the mRNA construct.

The C-terminal domain of Rv0125/Mtb32A (Mtb32A_{Cterm}, residues 224-355, SEQ ID NO. 220) was added to the 5′ end of the mRNA construct. Mtb32A is part of the M72/AS01_{E} vaccine candidate that has been shown to be protective in mice (Gene Ther. 2012 May; 19(5):570-5, PMID: 21956689) and the M72 vaccine was efficacious in a phase 2 clinical trial (N Engl J Med. 2019 Dec. 19; 381(25):2429-2439, PMID: 31661198). Additionally, the C-terminus of Mtb32A has been shown to promote bacterial expression of fusion proteins when placed at the N-terminus (Protein Expr Purif. 2003 July; 30(1):124-33, PMID: 12821330); we have not confirmed whether Mtb32A_{Cterm} increases mammalian expression of proteins encoded by an mRNA vaccine. Mtb32A_{Cterm} also contains the immunodominant MHC class I epitope H-2 D^{b}/GAPINSATAM (SEQ ID NO: 246) (309-318) (J Immunol. 2004 Jun. 15; 172(12):7618-28, PMID: 15187142).

A TCR cluster present in LTBI individuals was identified that recognized an “AANR” (SEQ ID NO: 245) motif present in the majority of PPE family proteins (68 family members), which are known to be immunogenic (Nat Biotechnol. 2020 October; 38(10):1194-1202, PMID: 32341563). This motif and surrounding residues have relatively high sequence homology. Importantly, peptides from multiple PPE proteins containing this motif were able to stimulate a single TCR clone from this TCR cluster, indicating that a single T cell clone has the ability to recognize multiple Mtb PPE proteins and could be broadly protective. While the 1st generation HLA-II mRNA (SEQ ID NO. 20 for the antigenic sequence) encodes for a segment of PPE18/MTB39A that contains the sequence variation AENR (SEQ ID NO: 247) within the “AANR motif” (SEQ ID NO: 245), we focused on the 30 out of 68 PPE proteins with the exact AANR sequence (SEQ ID NO: 245). The sequence plus/minus 10 residues on either side of AANR (SEQ ID NO: 245) were extracted. These 24mer peptide sequences were input into the HLA class II-restricted epitope predictor NetMHCIIpan EL (Immune Epitope Database, www.IEDB.org) and the 7-allele method (J Immunol Methods. 2015 July:422:28-34, PMID: 25862607) was used to identify 15 residue epitopes with broad HLA class II coverage. High affinity putative epitopes with a percentile rank of 20 or less were kept and input into the algorithm PopCover-2.0 to prioritize epitope selection with the broadest global HLA-II coverage. We identified the AANR (SEQ ID NO: 245) motifs within Rv1802/PPE30 (SEQ ID NO. 221) and RV1802/PPE40 (SEQ ID NO. 222) as potentially broadly recognized epitopes, and while both contain the same “VVAANR” (SEQ ID NO: 244) core, they differ in their flanking residues and thus may further increase the breadth of PPE-family proteins recognized by T cells.

The DPPS-NH₄, PEG-DMG and KC3-OA were kept constant at 5 mol %, 1.5 mol %, and 48 mol % respectively with an N/P ratio of 5.25. All mRNAs used N1-methylpseudouridine (NIMeU). The antigen encoded by two 1^{st} generation mRNA constructs consisted of 7 Mtb proteins and 10 minimal epitopes (polypeptide sequence corresponds to SEQ ID NO. 20) separated by spacers: 5 proteins were included in their entirety (ExsH, EsxW, EsxV, EsxA, EsxB), 2 in partial (Ag85B, Mtb39a), and a set of ten 15-mer epitopes were encoded at the 3′ end. One construct used an HLA-I signal peptide and terminal domain (sec/MITD; SEQ ID NOs. 37 and 38), while the other construct used a human LAMP-1 signal peptide and terminal domain (SEQ ID NOs. 39 and 40). The antigen encoded by the 2^{nd} generation mRNA had some overlap with the 1^{st} generation construct but added MTB32A, replaced EsxH/TB10.4 with PE13, and replaced the 10 C-terminal epitopes with two 18mer epitopes derived from PPE protein family members (Nat Biotechnol. 2020 October; 38(10):1194-1202, PMID: 32341563) and used the sec/MITD signal peptide (Table 63 for the individual polypeptide components, SEQ ID NOs. 2-7, 31, 220-222). The biophysical characteristics of the mRNA-LNPs are shown below in Table 59. The DSPC and cholesterol were varied, with the DSPC concentration being either 5 or 15 mol %, and the cholesterol concentration being either 40.5 or 30.5 mol %, respectively in the KC3OA LNP formulations. The specific formulation is indicated in the ICL, mol % PL column “ICL, mol % PL” of Table 59 with the 5 mol % DSPC and 40.5 mol % cholesterol formulation referred to as “KC3-OA, 10”, and the 15 mol % DSPC and 30.5 mol % cholesterol formulation referred to as “KC3-OA, 20”, where the 10 and 20 refer to the mol % of phospholipid in the LNP formulation.

LNPs were prepared as described in Example 9, characterized for particle size and zeta potential as described in Example 10, mRNA encapsulation efficiency and mRNA concentration as described in Example 10 and evaluated for antigen-specific CD4- and CD8-T cell responses in the spleens of CB6F1 mice.

TABLE 59
Physicochemical properties and characterization of 5 mol % DPPS-targeted KC3-OA LNPs.
LNPs were prepared with the formulation KC3-OA/DSPC/DPPS-NH₄/Chol/PEG-DMG
(48/5/5/40.5/1.5 mol %) containing a total of 10 mol % phospholipid (PL) or KC3- OA/DSPC/
DPPS-NH₄/Chol/PEG-DMG (48/15/5/30.5/1.5 mol % containing a total of 20
mol % phospholipid), as indicated below. Particle size refers to the Z-Average particle size.
Post-F/T refers to samples that were measured after a freeze/thaw event, and Zeta refers to
zeta potential measurements at either pH 5 or pH 7 in units of mV. mRNA encapsulation
efficiency is referred to as (E.E, %).

| | | | Particle Size | | | |
|---|---|---|---|---|---|---|
| ICL, | | Particle | (nm) | | Zeta | Zeta pH |
| mol % PL | mRNA | Size (nm) | Post-F/T | E.E, % | pH 5 | 7 |
| KC3-OA, | SEQ ID NOs. 37 and | 109.6 | 111.1 | 93.3 ± 9.3 | 14.94 | −0.2481 |
| 10 | 38 | | | | | |
| KC3-OA, | SEQ ID NOs. 39 and | 109.1 | 108.2 | 93.8 ± 10.1 | 13.95 | −1.311 |
| 10 | 40 | | | | | |
| KC3-OA, | SEQ ID NOs. 230 | 105.5 | 105.4 | 94.2 ± 5.8 | 19.27 | −0.8429 |
| 10 | and 231 | | | | | |
| KC3-OA, | SEQ ID NOs. 230 | 90.95 | 89.77 | 92.4 ± 6.7 | 19.43 | −1.803 |
| 20 | and 231 | | | | | |

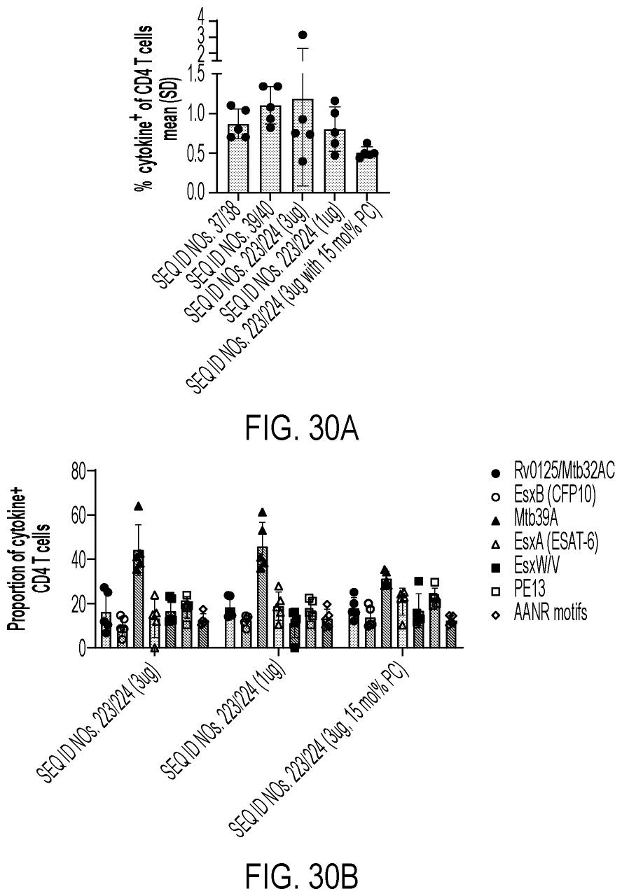

CD4 T cell responses to the 1^{st} and 2^{nd} generation HLA-II Mtb vaccine constructs are shown in FIGS. 30A-30B. CB6F1 mice were immunized i.m. with the indicated μg of mRNA encapsulated in KC3-OA/DPPS LNPs. Mice were boosted 4 weeks later, spleens were harvested and dissociated 7 days later, and cells were stimulated in separate wells with overlapping peptide pools covering each distinct antigenic encoded by the mRNA. Antigen-specific CD4 and CD8 T cells were defined as cells expressing IFN-γ, TNF-α, IL-2, or combinations thereof. No antigen-specific IL-17A production was observed (not shown). SEQ ID NOs. 37 and 38, mRNA encoding the 1^{st} generation construct with sec/MITD targeting. SEQ ID Nos. 39 and 40, mRNA encoding the 1^{st} generation construct with LAMP-1 targeting. SEQ ID NOs. 223 and 224, mRNA encoding the 2^{nd} generation construct with sec/MITD targeting. SEQ ID NOs. 225 and 226, another possible mRNA encoding a 2^{nd} generation construct with LAMP-1 targeting. One 2^{nd} generation mRNA group was vaccinated with mRNA formulated with KC3-OA/DPPS LNPs containing an increased amount of 15 mol % DSPC (at the expense of cholesterol) versus the typical 5 mol % DSPC. The cumulative total of the CD4 T cell response (sum of all individual peptide pools minus the background) is shown in FIG. 30A. The peptide pool for Ag85B was unintentionally left out of stimulations for the 2^{nd} generation mRNA (SEQ ID NOs. 223/224), so the reported magnitude of the total CD4 T cell response is smaller than in actuality. The proportion of total vaccine-induced CD4 T cell responses to individual or subsets of Mtb antigens encoded by the mRNA is shown in FIG. 30B. Data correspond to cumulative T cell responses shown in (FIG. 30A) and are normalized to 100%.

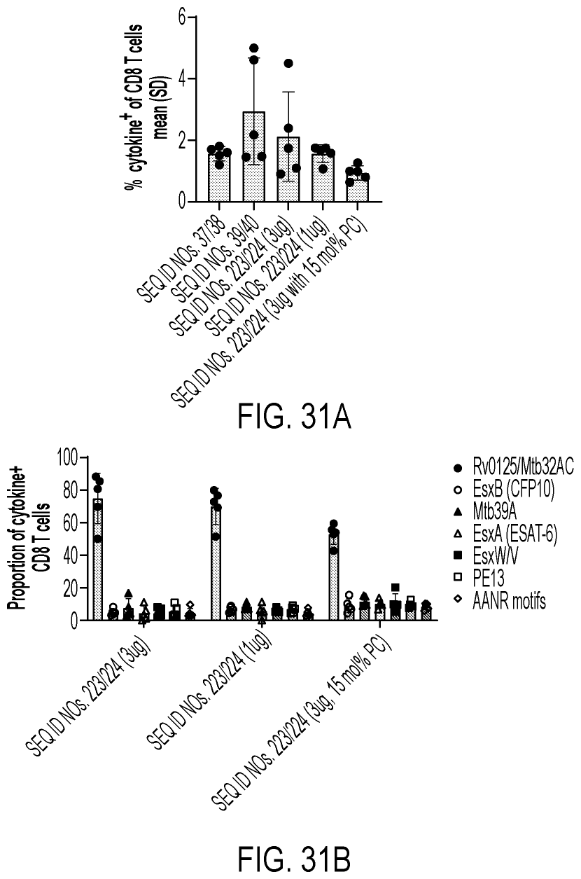

CD8 T cell responses to a 2^{nd} generation HLA-II Mtb vaccine construct FIGS. 31A-31B. Data are from the same experiment shown in FIGS. 30A-30B. The cumulative total of the CD8 T cell response (sum of all individual peptide pools minus the background) FIG. 31A. The proportion of total vaccine-induced CD8 T cell responses to individual or subsets of Mtb antigens encoded by the mRNA FIG. 31B. Data correspond to cumulative T cell responses shown in (FIG. 31A) and are normalized to 100

Altogether, this study demonstrates that the new antigens included in the 2^{nd} generation mRNA (Mtb32A_{Cterminus}, PE13, and AANR motif (SEQ ID NO: 245) epitopes) are translated, processed and presented on MHC class I and class II molecules by antigen-presenting cells. There is evidence that Mtb32A (part of the M72 vaccine candidate) and PE13 are protective in humans (Nat Med. 2023 January; 29(1):258-269, PMID: 36604540; N Engl J Med. 2019 Dec. 19; 381(25):2429-2439, PMID 31661198), and T cells with these specificities, in addition to the other antigens, may be protective against Mtb infection or progression to active disease. Additionally, KC3-OA/DPPS LNPs formulated with DSPC increased from the typical 5 mol % to 15 mol % (shown in Table 59 as 10 and 20 mol % PL, respectively) were immunogenic and induced CD4 and CD8 T cell responses. Increasing the mol % PL may be advantageous in terms of improved biophysical characteristics, stability, and in vivo immunogenicity.

TABLE 63
Mtb polypeptide antigens included in an mRNA vaccine construct. Sequences are
derived from the H37Rv reference strain.

| | Gene/ | Amino | |
|---|---|---|---|
| SEQ ID | protein | acids | Protein sequence |
| SEQ ID | Rv0125 | 224-355 | TAASDNFQLSQGGQGFAIPIGQAMAIAGQIRSGG |
| NO. 220 | MTB32Ac- | | GSPTVHIGPTAFLGLGVVDNNGNGARVQRVVG |
| | ₜₑᵣₘ | | SAPAASLGISTGDVITAVDGAPINSATAMADAL |
| | | | NGHHPGDVISVTWQTKSGGTRTGNVTLAEGPP |
| | | | A |
| SEQ ID | Rv3874 | 2-100 | AEMKTDAATLAQEAGNFERISGDLKTQIDQVES |
| NO. 6 | EsxB/CFP10 | | TAGSLQGQWRGAAGTAAQAAVVRFQEAANKQ |
| | | | KQELDEISTNIRQAGVQYSRADEEQQQALSSQM |
| | | | GF |
| SEQ ID | Rv1196 | 2-249 | VDFGALPPEINSARMYAGPGSASLVAAAQMWD |
| NO. 3 | Mtb39A | | SVASDLFSAASAFQSVVWGLTVGSWIGSSAGLM |
| | | | VAAASPYVAWMSVTAGQAELTAAQVRVAAAA |
| | | | YETAYGLTVPPPVIAENRAELMILIATNLLGQNT |
| | | | PAIAVNEAEYGEMWAQDAAAMFGYAAATATA |
| | | | TATLLPFEEAPEMTSAGGLLEQAAAVEEASDTA |
| | | | AANQLMNNVPQALQQLAQPTQGTTPSSKLGGL |
| | | | WKTVSPHRSPISNMVSMANNHM |
| SEQ ID | Rv3875 | 2-95 | TEQQWNFAGIEAAASAIQGNVTSIHSLLDEGKQ |
| NO. 7 | EsxA/ESAT- | | SLTKLAAAWGGSGSEAYQGVQQKWDATATEL |
| | 6 | | NNALQNLARTISEAGQAMASTEGNVTGMFA |
| SEQ ID | Rv3620c | 2-98 | TSRFMTDPHAMRDMAGRFEVHAQTVEDEARR |
| NO. 5 | EsxW | | MWASAQNISGAGWSGMAEATSLDTMTQMNQA |
| | | | FRNIVNMLHGVRDGLVRDANNYEQQEQASQQI |
| | | | LSS |
| SEQ ID | Rv1195 | 2-99 | SFVMAYPEMLAAAADTLQSIGATTVASNAAAA |
| NO. 31 | PE13 | | APTTGVVPPAADEVSALTAAHFAAHAAMYQSV |
| | | | SARAAAIHDQFVATLASSASSYAATEVANAAAA |
| | | | S |
| SEQ ID | Rv3619c | 2-94 | TINYQFGDVDAHGAMIRAQAGSLEAEHQAIISD |
| NO. 4 | EsxV | | VLTASDFWGGAGSAACQGFITQLGRNFQVIYEQ |
| | | | ANAHGQKVQAAGNNMAQTDSAVGSSWA |
| SEQ ID | Rv1886c | 111-200 | PVGGQSSFYSDWYSPACGKAGCQTYKWETFLT |
| NO. 2 | Ag85B | | SELPQWLSANRAVKPTGSAAIGLSMAGSSAMIL |
| | | | AAYHPQQFIYAGSLSALLDPSQGMG |
| SEQ ID | Rv1802 | 103-120 | TVPPPVVAANRAELAVLA |
| NO. 221 | PPE30 | | |
| SEQ ID | Rv2356c | 108-125 | MVDPVVVAANRSAFVQLV |
| NO. 222 | PPE40 | | |

SEQ ID NO. 223. Amino acid sequence of a synthetic antigen for an Mtb vaccine. Contains
concatenated polypeptides listed in Table 63, each separated by a GPGPG linker (SEQ ID NO:
228). The antigenic polypeptide is flanked on the N-terminus by the sec signal peptide and the
C-terminus by MITD. The N- and C-terminal signal domains are underlined.
MRVTAPRTLILLLSGALALTETWATAASDNFQLSQGGQGFAIPIGQAMAIAGQIRSGGGSPTVH
IGPTAFLGLGVVDNNGNGARVQRVVGSAPAASLGISTGDVITAVDGAPINSATAMADALNGHHP
GDVISVTWQTKSGGTRTGNVTLAEGPPAGPGPGAEMKTDAATLAQEAGNFERISGDLKTQIDQV
ESTAGSLQGQWRGAAGTAAQAAVVRFQEAANKQKQELDEISTNIRQAGVQYSRADEEQQQALSS
QMGFGPGPGVDFGALPPEINSARMYAGPGSASLVAAAQMWDSVASDLFSAASAFQSVVWGLTVG
SWIGSSAGLMVAAASPYVAWMSVTAGQAELTAAQVRVAAAAYETAYGLTVPPPVIAENRAELMI
LIATNLLGQNTPAIAVNEAEYGEMWAQDAAAMFGYAAATATATATLLPFEEAPEMTSAGGLLEQ
AAAVEEASDTAAANQLMNNVPQALQQLAQPTQGTTPSSKLGGLWKTVSPHRSPISNMVSMANNH
MGPGPGTEQQWNFAGIEAAASAIQGNVTSIHSLLDEGKQSLTKLAAAWGGSGSEAYQGVQQKWD
ATATELNNALQNLARTISEAGQAMASTEGNVTGMFAGPGPGTSRFMTDPHAMRDMAGRFEVHAQ
TVEDEARRMWASAQNISGAGWSGMAEATSLDTMTQMNQAFRNIVNMLHGVRDGLVRDANNYEQQ
EQASQQILSSGPGPGSFVMAYPEMLAAAADTLQSIGATTVASNAAAAAPTTGVVPPAADEVSAL
TAAHFAAHAAMYQSVSARAAAIHDQFVATLASSASSYAATEVANAAAASGPGPGTINYQFGDVD
AHGAMIRAQAGSLEAEHQAIISDVLTASDFWGGAGSAACQGFITQLGRNFQVIYEQANAHGQKV
QAAGNNMAQTDSAVGSSWAGPGPGPVGGQSSFYSDWYSPACGKAGCQTYKWETFLTSELPQWLS
ANRAVKPTGSAAIGLSMAGSSAMILAAYHPQQFIYAGSLSALLDPSQGMGGPGPGTVPPPVVAA
NRAELAVLAGPGPGMVDPVVVAANRSAFVQLVGIVAGLAVLAVVVIGAVVAAVMCRRKSSGGKG
GSYSQAACSDSAQGSDVSLTA
SEQ ID NO. 224. Codon-optimized mRNA forward open reading frame sequence corresponding
to the polypeptide in SEQ ID NO. 223. The sequence contains 3′ and 5′ HBB UTRs (SEQ ID NO.
32). N1-methylpseudouridine is substituted for all uridines (U) during mRNA synthesis.
ACAUUUGCUUCUGACACAACUGUGUUCACUAGCAACCUCAAACAGCCACCAUGCGCGUGACUGC
GCCUAGGACCCUGAUUCUGCUCCUGUCGGGUGCCCUCGCUCUGACCGAAACCUGGGCCACCGCC
GCGAGCGACAAUUUUCAGUUAAGCCAGGGUGGCCAGGGAUUUGCCAUCCCGAUCGGACAGGCCA
UGGCUAUCGCGGGCCAGAUCAGAUCCGGAGGCGGAUCCCCGACUGUACACAUCGGUCCGACCGC
CUUCUUGGGACUGGGAGUGGUGGACAACAACGGGAAUGGCGCACGGGUGCAGAGGGUCGUGGGC
UCGGCACCAGCCGCCUCUCUGGGAAUCAGCACCGGGGACGUUAUCACCGCUGUGGAUGGUGCCC
CCAUCAAUUCCGCCACGGCGAUGGCCGAUGCCCUGAACGGCCAUCACCCCGGGGAUGUGAUAUC
CGUGACCUGGCAGACCAAGUCCGGCGGUACAAGGACCGGCAACGUCACUCUGGCCGAGGGUCCU
CCUGCGGGCCCUGGUCCUGGUGCUGAGAUGAAAACCGACGCCGCGACCCUGGCCCAGGAAGCCG
GAAACUUCGAGCGGAUUUCCGGAGAUCUUAAGACUCAGAUCGACCAGGUCGAAUCCACCGCCGG
AUCGCUCCAGGGACAGUGGAGAGGCGCCGCGGGAACCGCGGCCCAGGCCGCCGUCGUGCGGUUC
CAAGAGGCCGCCAACAAGCAGAAGCAGGAACUGGACGAAAUUUCCACUAACAUUCGCCAAGCUG
GCGUGCAGUACUCGAGAGCCGAUGAAGAACAGCAGCAAGCCCUCUCCUCACAAAUGGGUUUCGG
ACCUGGGCCCGGCGUGGACUUCGGAGCCCUGCCACCUGAAAUCAACUCCGCCCGGAUGUACGCA
GGACCUGGAUCCGCCAGCCUGGUGGCGGCCGCGCAGAUGUGGGAUAGCGUGGCCUCCGACCUGU
UCAGCGCGGCUUCAGCUUUCCAAAGCGUGGUCUGGGGACUGACUGUGGGAUCCUGGAUCGGAUC
AAGCGCAGGCCUCAUGGUGGCCGCGGCGUCCCCUUACGUGGCCUGGAUGAGCGUGACCGCCGGC
CAGGCAGAACUGACCGCAGCCCAAGUCCGCGUGGCCGCAGCCGCCUACGAGACUGCCUACGGUC
UGACGGUGCCGCCGCCAGUGAUCGCCGAGAACAGAGCAGAGCUCAUGAUCCUCAUCGCGACCAA
CCUACUGGGCCAGAACACUCCGGCGAUUGCCGUGAACGAAGCCGAAUAUGGGGAGAUGUGGGCU
CAGGACGCUGCAGCCAUGUUCGGAUAUGCGGCCGCGACUGCUACCGCCACCGCCACUCUGCUGC
CGUUCGAGGAGGCCCCCGAAAUGACUUCCGCGGGAGGCCUGUUGGAGCAAGCUGCCGCGGUGGA
GGAAGCUAGCGACACCGCCGCAGCCAACCAGCUCAUGAACAACGUGCCACAGGCCCUGCAGCAG
CUGGCCCAGCCCACUCAAGGGACCACCCCGAGCUCAAAGCUGGGCGGUCUGUGGAAAACCGUGU
CCCCCCACCGCUCGCCCAUUUCCAACAUGGUGUCAAUGGCGAACAACCACAUGGGUCCUGGACC
UGGAACGGAGCAGCAGUGGAACUUCGCCGGGAUCGAAGCCGCCGCCUCGGCUAUCCAAGGGAAU
GUCACCUCGAUCCAUUCCCUUCUGGACGAAGGAAAGCAGUCCCUGACCAAGCUUGCGGCAGCCU
GGGGCGGAAGCGGCAGCGAAGCCUACCAGGGCGUGCAGCAAAAAUGGGACGCAACCGCCACUGA
GCUGAACAACGCCCUCCAAAACCUGGCUAGAACUAUUUCCGAGGCCGGACAGGCUAUGGCCAGC
ACCGAGGGCAACGUGACCGGGAUGUUCGCUGGCCCUGGGCCGGGCACCUCGCGGUUCAUGACCG
AUCCUCAUGCUAUGCGGGAUAUGGCUGGACGGUUCGAGGUGCACGCCCAAACUGUGGAGGACGA
GGCCCGCCGGAUGUGGGCCAGCGCGCAGAACAUCUCGGGGGCCGGCUGGUCCGGAAUGGCCGAA
GCCACCUCCCUCGAUACCAUGACCCAGAUGAACCAGGCCUUCCGGAACAUUGUGAACAUGCUGC
AUGGAGUGCGGGACGGCCUGGUCCGGGACGCCAACAAUUACGAGCAGCAGGAGCAGGCCUCUCA
GCAAAUCCUGUCCAGCGGGCCGGGGCCUGGAUCAUUCGUGAUGGCCUACCCUGAGAUGCUGGCA
GCCGCGGCCGACACCCUGCAAUCCAUUGGCGCCACUACCGUGGCAUCGAACGCGGCAGCCGCCG
CACCGACUACCGGCGUGGUGCCGCCCGCCGCUGAUGAAGUGUCCGCCCUUACUGCCGCCCACUU
CGCCGCCCACGCCGCGAUGUACCAGUCCGUGUCCGCGCGCGCCGCCGCUAUCCACGAUCAGUUC
GUCGCCACACUGGCCAGCUCAGCCUCCUCAUACGCCGCUACUGAAGUGGCAAACGCAGCCGCUG
CAUCCGGCCCGGGACCCGGAACGAUCAACUACCAGUUUGGCGACGUGGACGCGCACGGGGCGAU
GAUCAGGGCUCAAGCGGGGUCCCUCGAGGCCGAACACCAAGCGAUUAUCUCGGAUGUCCUCACC
GCGUCCGACUUCUGGGGUGGAGCCGGAAGCGCCGCCUGCCAAGGUUUCAUCACCCAACUGGGUC
GCAACUUUCAAGUCAUCUACGAACAGGCCAACGCCCACGGACAGAAGGUCCAAGCCGCCGGAAA
CAACAUGGCACAGACCGACUCCGCGGUCGGAUCGAGCUGGGCCGGACCCGGGCCAGGUCCCGUG
GGCGGACAGAGCUCGUUCUAUUCUGACUGGUACUCACCCGCAUGUGGAAAGGCCGGGUGCCAGA
CCUACAAAUGGGAAACCUUCUUGACUUCCGAGCUGCCUCAGUGGUUGUCCGCCAAUCGGGCCGU
GAAGCCGACCGGCUCGGCGGCCAUCGGCCUCUCCAUGGCCGGCUCGUCCGCGAUGAUUCUUGCC
GCCUACCACCCGCAGCAGUUCAUCUACGCCGGGUCCCUCUCGGCACUGCUGGACCCCAGUCAGG
GAAUGGGCGGACCCGGACCGGGAACUGUCCCGCCCCCGGUGGUGGCUGCUAACCGCGCCGAACU
UGCCGUGCUGGCCGGUCCAGGGCCGGGAAUGGUGGACCCAGUGGUGGUGGCAGCGAACAGAUCC
GCAUUCGUGCAACUGGUCGGCAUUGUGGCCGGACUGGCCGUGUUGGCCGUCGUCGUGAUCGGCG
CUGUGGUCGCCGCCGUGAUGUGCCGAAGAAAGUCCAGCGGUGGAAAGGGGGGAUCCUACUCACA
AGCAGCCUGCAGCGACUCAGCGCAGGGGUCGGACGUGUCACUGACAGCAGCUCGCUUUCUUGCU
GUCCAAUUUCUAUUAAAGGUUCCUUUGUUCCCUAAGUCCAACUACUAAACUGGGGGAUAUUAUG
AAGGGCCUUGAGCAUCUGGAUUCUGCCUAAUAAAAAACAUUUAUUUUCAUUGCAA
SEQ ID NO. 225. Amino acid sequence of a synthetic antigen for an Mtb vaccine. Contains
concatenated polypeptides listed in Table 63, each separated by a GPGPG linker (SEQ ID NO:
228). The antigenic polypeptide is flanked on the N-terminus by the LAMP-1 signal peptide (SEQ
ID NO. 21) and TM/CT on the C-terminus (SEQ ID NO. 22). The N- and C-terminal domains are
underlined.
MAAPGSARRPLLLLLLLLLLGLMHCASATAASDNFQLSQGGQGFAIPIGQAMAIAGQIRSGGGS
PTVHIGPTAFLGLGVVDNNGNGARVQRVVGSAPAASLGISTGDVITAVDGAPINSATAMADALN
GHHPGDVISVTWQTKSGGTRTGNVTLAEGPPAGPGPGAEMKTDAATLAQEAGNFERISGDLKTQ
IDQVESTAGSLQGQWRGAAGTAAQAAVVRFQEAANKQKQELDEISTNIRQAGVQYSRADEEQQQ
ALSSQMGFGPGPGVDFGALPPEINSARMYAGPGSASLVAAAQMWDSVASDLFSAASAFQSVVWG
LTVGSWIGSSAGLMVAAASPYVAWMSVTAGQAELTAAQVRVAAAAYETAYGLTVPPPVIAENRA
ELMILIATNLLGQNTPAIAVNEAEYGEMWAQDAAAMFGYAAATATATATLLPFEEAPEMTSAGG
LLEQAAAVEEASDTAAANQLMNNVPQALQQLAQPTOGTTPSSKLGGLWKTVSPHRSPISNMVSM
ANNHMGPGPGTEQQWNFAGIEAAASAIQGNVTSIHSLLDEGKQSLTKLAAAWGGSGSEAYQGVQ
QKWDATATELNNALQNLARTISEAGQAMASTEGNVTGMFAGPGPGTSRFMTDPHAMRDMAGRFE
VHAQTVEDEARRMWASAQNISGAGWSGMAEATSLDTMTQMNQAFRNIVNMLHGVRDGLVRDANN
YEQQEQASQQILSSGPGPGSFVMAYPEMLAAAADTLQSIGATTVASNAAAAAPTTGVVPPAADE
VSALTAAHFAAHAAMYQSVSARAAAIHDQFVATLASSASSYAATEVANAAAASGPGPGTINYQF
GDVDAHGAMIRAQAGSLEAEHQAIISDVLTASDFWGGAGSAACQGFITQLGRNFQVIYEQANAH
GQKVQAAGNNMAQTDSAVGSSWAGPGPGPVGGQSSFYSDWYSPACGKAGCQTYKWETFLTSELP
QWLSANRAVKPTGSAAIGLSMAGSSAMILAAYHPQQFIYAGSLSALLDPSQGMGGPGPGTVPPP
VVAANRAELAVLAGPGPGMVDPVVVAANRSAFVQLVLIPIAVGGALAGLVLIVLIAYLVGRKRS
HAGYQTI
SEQ ID NO. 226. Codon-optimized mRNA forward open reading frame sequence corresponding
to the polypeptide in SEQ ID NO. 225. The sequence contains 3′ and 5′ HBB UTRs (SEQ ID NO.
32). N1-methylpseudouridine is substituted for all uridines (U) during mRNA synthesis.
ACAUUUGCUUCUGACACAACUGUGUUCACUAGCAACCUCAAACAGCCACCAUGGCGGCUCCGGG
AAGCGCACGACGCCCGCUGCUGCUCUUGUUGCUGCUCCUCCUGCUGGGUCUGAUGCACUGCGCC
UCGGCCACCGCCGCGAGCGACAAUUUUCAGUUAAGCCAGGGUGGCCAGGGAUUUGCCAUCCCGA
UCGGACAGGCCAUGGCUAUCGCGGGCCAGAUCAGAUCCGGAGGCGGAUCCCCGACUGUACACAU
CGGUCCGACCGCCUUCUUGGGACUGGGAGUGGUGGACAACAACGGGAAUGGCGCACGGGUGCAG
AGGGUCGUGGGCUCGGCACCAGCCGCCUCUCUGGGAAUCAGCACCGGGGACGUUAUCACCGCUG
UGGAUGGUGCCCCCAUCAAUUCCGCCACGGCGAUGGCCGAUGCCCUGAACGGCCAUCACCCCGG
GGAUGUGAUAUCCGUGACCUGGCAGACCAAGUCCGGCGGUACAAGGACCGGCAACGUCACUCUG
GCCGAGGGUCCUCCUGCGGGCCCUGGUCCUGGUGCUGAGAUGAAAACCGACGCCGCGACCCUGG
CCCAGGAAGCCGGAAACUUCGAGCGGAUUUCCGGAGAUCUUAAGACUCAGAUCGACCAGGUCGA
AUCCACCGCCGGAUCGCUCCAGGGACAGUGGAGAGGCGCCGCGGGAACCGCGGCCCAGGCCGCC
GUCGUGCGGUUCCAAGAGGCCGCCAACAAGCAGAAGCAGGAACUGGACGAAAUUUCCACUAACA
UUCGCCAAGCUGGCGUGCAGUACUCGAGAGCCGAUGAAGAACAGCAGCAAGCCCUCUCCUCACA
AAUGGGUUUCGGACCUGGGCCCGGCGUGGACUUCGGAGCCCUGCCACCUGAAAUCAACUCCGCC
CGGAUGUACGCAGGACCUGGAUCCGCCAGCCUGGUGGCGGCCGCGCAGAUGUGGGAUAGCGUGG
CCUCCGACCUGUUCAGCGCGGCUUCAGCUUUCCAAAGCGUGGUCUGGGGACUGACUGUGGGAUC
CUGGAUCGGAUCAAGCGCAGGCCUCAUGGUGGCCGCGGCGUCCCCUUACGUGGCCUGGAUGAGC
GUGACCGCCGGCCAGGCAGAACUGACCGCAGCCCAAGUCCGCGUGGCCGCAGCCGCCUACGAGA
CUGCCUACGGUCUGACGGUGCCGCCGCCAGUGAUCGCCGAGAACAGAGCAGAGCUCAUGAUCCU
CAUCGCGACCAACCUACUGGGCCAGAACACUCCGGCGAUUGCCGUGAACGAAGCCGAAUAUGGG
GAGAUGUGGGCUCAGGACGCUGCAGCCAUGUUCGGAUAUGCGGCCGCGACUGCUACCGCCACCG
CCACUCUGCUGCCGUUCGAGGAGGCCCCCGAAAUGACUUCCGCGGGAGGCCUGUUGGAGCAAGC
UGCCGCGGUGGAGGAAGCUAGCGACACCGCCGCAGCCAACCAGCUCAUGAACAACGUGCCACAG
GCCCUGCAGCAGCUGGCCCAGCCCACUCAAGGGACCACCCCGAGCUCAAAGCUGGGCGGUCUGU
GGAAAACCGUGUCCCCCCACCGCUCGCCCAUUUCCAACAUGGUGUCAAUGGCGAACAACCACAU
GGGUCCUGGACCUGGAACGGAGCAGCAGUGGAACUUCGCCGGGAUCGAAGCCGCCGCCUCGGCU
AUCCAAGGGAAUGUCACCUCGAUCCAUUCCCUUCUGGACGAAGGAAAGCAGUCCCUGACCAAGC
UUGCGGCAGCCUGGGGCGGAAGCGGCAGCGAAGCCUACCAGGGCGUGCAGCAAAAAUGGGACGC
AACCGCCACUGAGCUGAACAACGCCCUCCAAAACCUGGCUAGAACUAUUUCCGAGGCCGGACAG
GCUAUGGCCAGCACCGAGGGCAACGUGACCGGGAUGUUCGCUGGCCCUGGGCCGGGCACCUCGC
GGUUCAUGACCGAUCCUCAUGCUAUGCGGGAUAUGGCUGGACGGUUCGAGGUGCACGCCCAAAC
UGUGGAGGACGAGGCCCGCCGGAUGUGGGCCAGCGCGCAGAACAUCUCGGGGGCCGGCUGGUCC
GGAAUGGCCGAAGCCACCUCCCUCGAUACCAUGACCCAGAUGAACCAGGCCUUCCGGAACAUUG
UGAACAUGCUGCAUGGAGUGCGGGACGGCCUGGUCCGGGACGCCAACAAUUACGAGCAGCAGGA
GCAGGCCUCUCAGCAAAUCCUGUCCAGCGGGCCGGGGCCUGGAUCAUUCGUGAUGGCCUACCCU
GAGAUGCUGGCAGCCGCGGCCGACACCCUGCAAUCCAUUGGCGCCACUACCGUGGCAUCGAACG
CGGCAGCCGCCGCACCGACUACCGGCGUGGUGCCGCCCGCCGCUGAUGAAGUGUCCGCCCUUAC
UGCCGCCCACUUCGCCGCCCACGCCGCGAUGUACCAGUCCGUGUCCGCGCGCGCCGCCGCUAUC
CACGAUCAGUUCGUCGCCACACUGGCCAGCUCAGCCUCCUCAUACGCCGCUACUGAAGUGGCAA
ACGCAGCCGCUGCAUCCGGCCCGGGACCCGGAACGAUCAACUACCAGUUUGGCGACGUGGACGC
GCACGGGGCGAUGAUCAGGGCUCAAGCGGGGUCCCUCGAGGCCGAACACCAAGCGAUUAUCUCG
GAUGUCCUCACCGCGUCCGACUUCUGGGGUGGAGCCGGAAGCGCCGCCUGCCAAGGUUUCAUCA
CCCAACUGGGUCGCAACUUUCAAGUCAUCUACGAACAGGCCAACGCCCACGGACAGAAGGUCCA
AGCCGCCGGAAACAACAUGGCACAGACCGACUCCGCGGUCGGAUCGAGCUGGGCCGGACCCGGG
CCAGGUCCCGUGGGCGGACAGAGCUCGUUCUAUUCUGACUGGUACUCACCCGCAUGUGGAAAGG
CCGGGUGCCAGACCUACAAAUGGGAAACCUUCUUGACUUCCGAGCUGCCUCAGUGGUUGUCCGC
CAAUCGGGCCGUGAAGCCGACCGGCUCGGCGGCCAUCGGCCUCUCCAUGGCCGGCUCGUCCGCG
AUGAUUCUUGCCGCCUACCACCCGCAGCAGUUCAUCUACGCCGGGUCCCUCUCGGCACUGCUGG
ACCCCAGUCAGGGAAUGGGCGGACCCGGACCGGGAACUGUCCCGCCCCCGGUGGUGGCUGCUAA
CCGCGCCGAACUUGCCGUGCUGGCCGGUCCAGGGCCGGGAAUGGUGGACCCAGUGGUGGUGGCA
GCGAACAGAUCCGCAUUCGUGCAACUGGUCCUGAUUCCUAUUGCGGUCGGCGGGGCUCUCGCCG
GGCUCGUGCUCAUUGUCCUGAUUGCCUACCUGGUCGGACGCAAGAGGUCCCAUGCGGGCUACCA
GACCAUCUGAUAAUAGGCUCGCUUUCUUGCUGUCCAAUUUCUAUUAAAGGUUCCUUUGUUCCCU
AAGUCCAACUACUAAACUGGGGGAUAUUAUGAAGGGCCUUGAGCAUCUGGAUUCUGCCUAAUAA
AAAACAUUUAUUUUCAUUGCAA
SEQ ID NO. 228. A non-immunogenic protein spacer to prevent junctional class II-restricted
neoantigen formation.
GPGPG

### Example 39. Impact of PL/Chol Ratio in PS-Targeted LNPs on Biophysical Characteristics and Transfection Efficiency in Murine DCs

The aim of this study was to explore the effect of varying the phospholipid/cholesterol ratio (PL/Chol ratio) in DPPS-targeted KC3-OA LNPs on biophysical characteristics and transfection efficiency in murine dendritic cells. The PL/Chol ratio was varied from 0.11-100 (5-50 mol % PL and 45.5-0.5 mol % Chol), while keeping the DPPS-NH₄, PEG-DMG and KC3-OA constant at 5 mol %, 1.5 mol %, and 48 mol % respectively with an N/P ratio of 5.25 using mCherry encoding mRNA. All mRNAs used N1-methylpseudouridine (N1MeU). An ALC-0315 based formulation composed of 46.3 mol % ALC-0315, 9.4 mol % DSPC, 42.7 mol % cholesterol and 1.56 mol % PEG-DMG with a N/P of 6.2 was also made and evaluated.

LNPs were prepared as described in Example 9, characterized for particle size and zeta potential as described in Example 10, mRNA encapsulation efficiency and mRNA concentration as described in Example 10 and evaluated for transfection efficiency in murine dendritic cells as described in Example 11.

TABLE 60
Physicochemical properties and characterization of DPPS-targeted KC3-OA LNPs with varying
PL/Chol ratios, described in terms of PL/Chol mol %/mol % ratio. Particle size refers
to the Z-Average particle size. Post-F/T refers to samples that were measured after
a freeze/thaw event, and Zeta refers to zeta potential measurements at either pH 5 or
pH 7 in units of mV. mRNA encapsulation efficiency is referred to as (E.E, %).

| | PL/Chol | Particle | Particle Size | | | |
|---|---|---|---|---|---|---|
| ICL | (mol %/mol %) | size (nm) | (nm) Post-F/T | E.E, % | Zeta pH 5 | Zeta pH 7 |
| ALC-0315 | 9.4/42.7 | 94.00 | 93.14 | 81.0 ± 4.5 | 5.44 | −4.45 |
| KC3-OA | 5/45.5 | 116.30 | 114.40 | 91.6 ± 10.9 | 12.76 | −2.31 |
| KC3-OA | 10/40.5 | 109.50 | 110.30 | 91.6 ± 10.7 | 17.33 | −1.18 |
| KC3-OA | 15/35.5 | 99.02 | 97.60 | 92.7 ± 1.5 | 11.22 | −1.13 |
| KC3-OA | 20/30.5 | 82.74 | 81.50 | 91.9 ± 2.6 | 14.54 | −2.47 |
| KC3-OA | 25/25.5 | 80.92 | 79.80 | 91.5 ± 2.3 | 17.40 | −3.11 |
| KC3-OA | 30/20.5 | 82.85 | 83.48 | 90.7 ± 1.2 | 12.41 | 0.08 |
| KC3-OA | 35/15.5 | 96.60 | 95.90 | 90.1 ± 2.5 | 19.81 | −0.43 |
| KC3-OA | 40/10.5 | 117.4 | 118.6 | 85.9 ± 2.5 | 18.47 | −0.45 |
| KC3-OA | 45/5.5 | 113.7 | 116.1 | 85.8 ± 2.9 | 13.94 | 1.62 |
| KC3-OA | 50/0.5 | 96.25 | 97.27 | 83.0 ± 2.5 | 9.82 | 1.45 |

KC3-OA formulations showed a dependence of PL/Chol on particle size. The Z-average LNP particle size was highest (>100 nm) at the lowest and highest PL/Chol ratios tested, but at intermediate ratios (15/35.5-35.5/15 mol %/mol %) the size was found to be less than 100 nm and smallest at a PL ratio of 25/25.5 (mol %/mol %). At higher PL/Chol ratios (at 40 mol % PL or more) the E.E, % was reduced below 90% while >90% E.E, % was found with all KC3-OA LNPs with <40 mol % PL. All particles were stable to freezing and thawing at −80° C. as judged by the small changes in Z-Ave particle sizes after F/T.

TABLE 61
mCherry expression in murine dendritic cells MutuDC
1940 following incubation of mCherry containing mRNA-
LNPs at 0.1 μg/ml mRNA concentration for 18 h.

| | PL/Chol | |
|---|---|---|
| ICL | (mol %/mol %) | Med FI |
| Untreated cells | | 84.3 ± 3.7 |
| ALC-0315 | 9.4/42.7 | 1181.0 ± 80.5 |
| KC3-OA | 5/45.5 | 772.7 ± 431.4 |
| KC3-OA | 10/40.5 | 10476.7 ± 1749.9 |
| KC3-OA | 15/35.5 | 11377.3 ± 2361.1 |
| KC3-OA | 20/30.5 | 3799.7 ± 578.5 |
| KC3-OA | 25/25.5 | 1272.3 ± 97.9 |
| KC3-OA | 30/20.5 | 404.7 ± 32.5 |
| KC3-OA | 35/15.5 | 126.3 ± 4.2 |
| KC3-OA | 40/10.5 | 108.0 ± 2.6 |
| KC3-OA | 45/5.5 | 101.7 ± 4.6 |
| KC3-OA | 50/0.5 | 105.0 ± 2.6 |

MutuDC1940 cells were plated and LNPs added to cell culture media as described in Example 11. A striking dependence on in vitro mCherry activity to PL/Chol was observed. It was unexpectedly found that the transfection efficiency could be further optimized by increasing the total phospholipid content to 15 mol %. For example, the most mCherry expression was found with LNPs consisting of PL/Chol ratios of 10/40.5 and 15/35.5 which were both ~100-fold higher than the 3 formulations tested with ≥40 mol % PL. In vitro mCherry expression varied with LNP particle size. For example, while the LNPs with 10/40.5, 15/35.5 PL/Chol had sizes ~110 and 98 nm respectively and had the highest mCherry expression, LNPs with larger or smaller sizes displayed lower mCherry expression.

### Example 40. Preparation of LNPs Containing Either HLA Class I or Class II-Directed Mtb mRNA with Varying Concentrations of Total Phospholipid

The aim of this study was to explore the effect of varying the phospholipid/cholesterol ratio (PL/Chol ratio) in DPPS-targeted KC3-OA LNPs on biophysical characteristics of the mRNA LNPs. We additionally sought to explore how the PL/Chol ratio affected in vivo T cell responses. The PL/Chol ratio was varied from 0.11-3.8 (5-40 mol % PL and 45.5-10.5 mol % Chol), while keeping the DPPS-NH₄, PEG-DMG and KC3-OA constant at 5 mol %, 1.5 mol %, and 48 mol % respectively with an N/P ratio of 5.25 using three different mRNA constructs. All mRNAs utilized N1-methylpseudouridine (i.e. chemically modified) except for a single formulation (SEQ ID NOs. 223/224 (unmodified uridine), Table 62). One mRNA encoded HLA class I-restricted epitopes shown in Table 49, SEQ ID NOs. 212 and 213; the second mRNA encoded HLA class I restricted epitopes shown in Table 48, SEQ ID NOs. 215 and 216; and the third mRNA encoded antigens shown in Table 63, SEQ ID NOs. 223 and 224. The biophysical characteristics of these mRNA-LNPs is shown in Table 62.

LNPs were prepared as described in Example 9, characterized for particle size and zeta potential as described in Example 10, mRNA encapsulation efficiency and mRNA concentration as described in Example 10. Physicochemical properties and characterization of 5 mol % DPPS-targeted KC3-OA LNPs. PL content refers to the total phospholipid (PL) content which is a combination of phosphatidylserine and phosphatidylcholine present in the formulation. Particle size refers to the Z-Average particle size. Post-F/T refers to samples that were measured after a freeze/thaw event, and Zeta refers to zeta potential measurements at either pH 5 or pH 7 in units of mV. mRNA encapsulation efficiency is referred to as (E.E, %).

SEQ ID NO. 212. Example of an amino acid sequence for an HLA class I “Mixed” antigen flanked
on the N-terminus by the sec signal peptide the MITD on the C-terminus. The N- and C-terminal
domains are underlined. The antigenic sequence corresponds to SEQ ID NO. 101.
MRVTAPRTLILLLSGALALTETWAFYVWDFAEKFKEDVINDFVSSYAVADPMGAAFDYAAEVLR
VPPRPYVMPHSPAAGAAIPAVVPELTPVAAAPPMLAVAIEKPVAPSVMDFPDSGTHSWERAVPG
RVVALSLLDAHIPQLVRVLDALTDKLADTDPLPVVFWERNDPTQQIVRAATTMATYVSSPDVLT
TYIAAADEVSTQVAIDELKINSSLLTSILTYFAAPVALAAYPITGKLAAAVDIRETFRMAVVGG
ALAYLVVKTLINATQLLKLLAKLATIATFEMRYLQSLPAHLRPGLDELATIDVAIPKLRGGTHP
TTTYKAFDWDQAYRKPITYIMYNYPAMLGYYQSGLSIVMAARALPLTSLTSAAERGPGQMLVAE
LIGDALFVQLHAPLRGVARGQTLVLYVEMAAQQHVVIEAYTAAVELMPTQVELADTRVATPSGL
DGPGMSTSAYDIGLFYRYAWQNPVFAFHDQFVQALRAKLLSSILTYHSELRALVEVYPISPPGF
PSLPYAMTLPPPVVAANRIQLLSREMTDPHAMRGFAIPIGQAMAMYAGPGSASLATYDKGILTV
GYTDDAGQTFVQELDEISTNIQAAEAFVAAYMPFLILAPQINFFYILAAYHPQQFIYAGSLSAL
LELENDNQLLYDYVYPGHAIWWLARGLEFFQDGEQFGELLFTNPTGAFTLAIYHPQQFVYIAVM
PPAPGMVFSRPGLPVEYLQVPSPSMGAAFQGAHARFGRASGVLPRYGLPPPWQPALRHPRSLFP
EFSELFAAFPSFAGLRPTFDTRLMRSRIDGTHQTLVAAWDKVMNLTYIPVVGHALSIRMWNQAA
LAMEVYQAETAVNTLFSQATDPLSLLIETVTQALQALTIPSFIPEDFTFLVHAASTRQALRSQF
NDTLNVYLLPLPNIPLLEYAARFITPVHPGYTATFLETPSQFFPFTGLNSLTLLDAHIPQLISA
QAAAIHEMFAFQYDGVNDFPKYPLNVFATANAIAGILFLHSGLIALPHTSPPPLILKSMVRDGQ
LTIKGSVTPAVSQFNARTADGINYRVAFPPEVHSAMLKELDEAVEAFGIVAGLAVLAVVVIGAV
VAAVMCRRKSSGGKGGSYSQAACSDSAQGSDVSLTA
SEQ ID NO. 213. Codon-optimized mRNA forward ORF sequence corresponding to the
polypeptide in SEQ ID NO. 212. The sequence contains 5′ and 3′ HBB UTRs (SEQ ID NO. 32). N1-
methylpseudouridine is substituted for all uridines (U) during mRNA synthesis.
ACAUUUGCUUCUGACACAACUGUGUUCACUAGCAACCUCAAACAGCCACCAUGAGAGUGACCGC
CCCGCGGACCCUCAUUCUGCUUCUGUCCGGAGCGCUCGCACUGACUGAGACUUGGGCAUUCUAC
GUCUGGGACUUCGCCGAGAAGUUCAAGGAAGAUGUGAUCAACGACUUCGUGAGCUCGUACGCUG
UGGCCGACCCUAUGGGAGCCGCCUUCGACUACGCCGCCGAAGUGCUCAGGGUCCCUCCGCGGCC
UUACGUGAUGCCACACUCCCCGGCCGCUGGGGCCGCUAUCCCCGCGGUGGUCCCUGAACUCACC
CCAGUCGCCGCAGCCCCCCCUAUGCUGGCCGUGGCCAUUGAGAAGCCUGUCGCCCCGAGCGUGA
UGGAUUUCCCGGAUUCCGGAACCCAUAGCUGGGAACGCGCUGUGCCCGGACGGGUGGUGGCAUU
GAGCCUGCUCGACGCCCACAUUCCCCAACUCGUGCGCGUCCUGGACGCCCUGACUGAUAAGCUG
GCCGACACCGAUCCCCUGCCCGUUGUGUUCUGGGAGAGGAACGAUCCAACUCAGCAAAUUGUCC
GGGCGGCUACCACUAUGGCAACAUACGUGUCGUCGCCGGACGUGCUUACAACCUACAUAGCGGC
GGCGGACGAAGUGUCUACCCAAGUGGCCAUCGACGAACUUAAGACCAACUCCAGUUUGCUGACC
UCCAUCCUGACCUACUUCGCGGCCCCCGUGGCCCUGGCUGCCUACCCUAUCACCGGAAAGCUCG
CCGCCGCGGUGGAUAUCAGAGAAACCUUCCGGAUGGCAGUCGUGGGUGGAGCCUUGGCCUACCU
GGUGGUCAAGACCCUGAUCAACGCCACGCAGUUGCUGAAGCUGCUCGCGAAGCUGGCGACCAUC
GCCACUUUCGAGAUGCGGUAUCUGCAGUCUCUGCCCGCUCACCUCCGGCCCGGGCUCGACGAGC
UGGCCACCAUCGACGUGGCGAUCCCGAAACUGAGAGGAGGCACCCACCCGACCACCACUUACAA
GGCAUUCGAUUGGGAUCAGGCCUACCGGAAGCCUAUUACCUACAUUAUGUAUAACUACCCGGCC
AUGCUGGGCUAUUACCAGUCGGGCCUGAGCAUCGUGAUGGCCGCACGGGCUCUGCCCCUGACCU
CCUUGACCUCGGCCGCCGAGCGCGGACCCGGACAGAUGUUGGUGGCAGAACUCAUCGGAGAUGC
ACUGUUCGUGCAACUGCACGCGCCGCUCCGCGGCGUGGCUCGAGGACAGACCCUGGUGCUCUAC
GUGGAAAUGGCCGCCCAGCAGCAUGUGGUCAUCGAGGCCUACACCGCCGCCGUGGAGCUGAUGC
CCACUCAAGUGGAGCUGGCAGACACUCGCGUGGCCACCCCGAGCGGCCUCGACGGCCCGGGAAU
GUCGACCUCCGCGUACGACAUCGGUCUGUUCUACCGCUACGCCUGGCAAAACCCUGUGUUCGCG
UUCCAUGACCAGUUCGUACAGGCUCUGCGCGCCAAACUGCUGUCCAGCAUUCUGACCUACCAUU
CCGAACUGCGGGCGCUUGUGGAGGUCUACCCCAUCUCCCCCCCGGGUUUCCCAUCAUUACCUUA
CGCCAUGACUCUCCCACCGCCCGUGGUCGCCGCGAAUCGCAUUCAGCUGCUAAGCCGCUUCAUG
ACCGACCCACACGCGAUGAGGGGAUUCGCCAUCCCCAUUGGGCAGGCCAUGGCUAUGUACGCUG
GCCCGGGAUCCGCCUCCCUGGCCACCUACGACAAGGGAAUCCUGACUGUGGGCUACACGGACGA
UGCAGGACAGACUUUCGUGCAAGAACUGGAUGAAAUCAGCACUAACAUCCAGGCAGCCGAGGCC
UUUGUGGCGGCCUACAUGCCUUUCCUGAUCCUCGCGCCGCAAAUCAAUUUCUUCUACAUUCUGG
CCGCUUACCACCCGCAGCAGUUCAUCUACGCCGGAUCGCUGUCAGCACUGCUGGAGCUGGAGAA
CGACAACCAGCUCCUCUACGACUACGUCUACCCUGGCCAUGCGAUUUGGUGGCUUGCUCGCGGC
UUGGAGUUUUUCCAAGACGGCGAACAGUUCGGUGAACUCCUGUUCACCAACCCUACGGGAGCCU
UCACUCUGGCCAUCUAUCACCCCCAACAGUUCGUGUAUAUCGCCGUGAUGCCCCCGGCCCCCGG
AAUGGUCUUUUCGCGGCCGGGCCUUCCGGUGGAGUAUCUCCAGGUCCCUUCCCCUUCGAUGGGG
GCCGCCUUCCAAGGCGCCCACGCACGGUUUGGUCGCGCGUCAGGAGUGCUGCCGCGAUACGGAC
UGCCCCCGCCAUGGCAGCCGGCCCUCCGCCACCCAAGAUCGCUGUUCCCCGAAUUCUCGGAGCU
CUUUGCUGCCUUCCCCUCCUUCGCCGGGCUUAGGCCAACCUUCGACACCCGGCUCAUGAGAUCG
CGCAUCGACGGUACUCACCAGACCCUCGUGGCCGCCUGGGACAAGGUCAUGAACCUGACUUACA
UCCCUGUCGUGGGACACGCCCUUUCCAUUCGGAUGUGGAACCAGGCAGCGCUGGCCAUGGAAGU
GUACCAGGCCGAAACCGCCGUGAACACCCUGUUUUCCCAAGCCACUGACCCGCUUUCCCUCCUU
AUCGAAACAGUGACCCAGGCACUGCAAGCUCUGACCAUUCCAAGCUUCAUCCCUGAGGAUUUCA
CUUUCCUGGUGCACGCCGCCAGCACCAGACAGGCCCUGAGAUCCCAGUUCAACGACACCCUGAA
CGUGUAUCUGCUGCCCCUGCCCAACAUUCCGCUCCUGGAAUACGCCGCUAGAUUCAUCACUCCU
GUGCAUCCUGGGUACACCGCCACCUUCCUGGAAACCCCGAGCCAGUUCUUCCCGUUCACGGGUU
UGAACUCCCUGACCCUGCUGGACGCUCAUAUUCCUCAACUCAUUAGCGCACAGGCCGCCGCCAU
UCACGAAAUGUUUGCCUUCCAAUACGACGGAGUGAAUGACUUUCCGAAGUACCCCCUCAACGUG
UUCGCCACCGCGAACGCCAUUGCGGGGAUCCUCUUCCUGCACUCCGGGCUGAUCGCCCUGCCUC
ACACUUCCCCGCCCCCACUGAUCCUUAAGUCAAUGGUCCGCGACGGACAGCUGACUAUCAAGGG
GUCCGUCACUCCCGCGGUGUCCCAGUUUAAUGCGCGCACUGCGGACGGCAUCAACUAUCGGGUG
GCGUUUCCGCCGGAAGUGCACUCAGCCAUGCUGAAGGAGUUGGACGAGGCGGUGGAGGCCUUCG
GAAUUGUGGCCGGCCUUGCCGUGCUCGCCGUGGUCGUGAUCGGCGCCGUGGUGGCCGCCGUCAU
GUGCCGGAGAAAGAGCUCAGGAGGAAAGGGUGGCAGCUACAGCCAGGCAGCAUGUUCAGAUUCG
GCCCAGGGCUCCGAUGUGUCCCUGACCGCCUGAUAAUAGGCUCGCUUUCUUGCUGUCCAAUUUC
UAUUAAAGGUUCCUUUGUUCCCUAAGUCCAACUACUAAACUGGGGGAUAUUAUGAAGGGCCUUG
AGCAUCUGGAUUCUGCCUAAUAAAAAACAUUUAUUUUCAUUGCAA
SEQ ID NO. 215. Example of an amino acid sequence for an HLA-I “Mtb-only” antigen flanked on
the N-terminus by the sec signal peptide the MITD on the C-terminus. The N- and C-terminal
domains are underlined.
MRVTAPRTLILLLSGALALTETWAWRHWVHALTRINLGLSPDEKYELDLHARVRRNPRGSYQIA
VVGLQLPPAATQTLPAAPVITAAMADPAADLIAPKTYCEELKGTDTGQACQIQMSDPAYNINIS
LPSYYPDQKSLENYIAQTRDKFLSAATSSTPREAPYELNITSATYQSAIPPRGTQAVVLKVYQN
AGGTHPTTTYKAFDWDQAYRKPITYDTLWQADTDPLPVVFPIVQGELSKQTGQQVSIAPNAGLD
PVNYQNFAVTNDGVIFFFNPGELLPEAAGPTQVLVPRSAIDSMLAAETDTEQEIALFTVTNPPR
TVSVSTLMDGRIDHVELSARVAWMSESQLASEILVIADLARQKAQSAQYAFILDRMSQQVDADE
HRVALLGWTADPIIGVQVRADRILALAVAASNNPELTTLTAALSGQLNPQVNLVDTLNSGQYTV
FAPTNAAFSKLPASTIDELKTNSSLLTSILTYNHIMPGEPNVAVKDLVRHFEQQVQPGRVVVMP
WDRHIAAGTEISLDLLDPIYKRKVLELAAALQAETAVNTLFEKLEPMAAASKPPTPPMVHAAST
RQALRAHFADGWNTFNLAEAEVFATRYSAAKNAAQQLVLSADNMREYLAAGAKERQRLATSLRN
AAKRLYAENPSARDQILPVYAEYQQRSEKVLTEYNNKAALDQQRQWILHMAKLSAAMAKQAQYV
AQLHVWARREHPTYEGVHTANATVYMIDTVLMASKGTTTKKYMAADYDKLFRATIADVLAEKEL
SHYNDIRAHTSVNAVNLEVLPAPEYSSAQRALSDADWHFIADPASRFYNLVLAFVRPVAVDLTY
IPVVGHALSASQFNDTLNVYLTAHNALGSSLHTAGVDLAKFFDPLTRGVLTRTPLMSQLIFIID
PTISAIDGLYDLLGIGIPNQGGILYSSLEYFEKALEELAAEQVGGQSQLGKLNPDVNLVDTLNG
GEYTVFAPTNAAFDKLPAATIDQLKTDAKLLSSILTYHVIAGQASPSRIDGTHQTLRRTAPAPP
WAKMMAVVGGALAYLVVKTLINATQLLKLLAKLGVSTANATVYMIDSVLMQPFFDPSASFPGIV
AGLAVLAVVVIGAVVAAVMCRRKSSGGKGGSYSQAACSDSAQGSDVSLTA
SEQ ID NO. 216. Codon-optimized mRNA forward open reading frame sequence
corresponding to the polypeptide in SEQ ID NO. 215. The sequence contains 3′ and 5′ HBB UTRs
(SEQ ID NO. 32). N1-methylpseudouridine is substituted for all uridines (U) during mRNA
synthesis.
ACAUUUGCUUCUGACACAACUGUGUUCACUAGCAACCUCAAACAGCCACCAUGCGCGUGACUGC
ACCCCGCACUUUAAUCUUGCUGCUGUCUGGAGCCCUCGCGCUGACCGAGACAUGGGCAUGGCGC
CACUGGGUCCACGCCCUCACCCGGAUCAACCUGGGACUGUCACCCGACGAGAAAUACGAACUGG
ACCUCCACGCCCGGGUCCGCCGGAACCCAAGAGGAUCCUACCAGAUUGCCGUCGUGGGACUGCA
GCUGCCCCCUGCGGCCACUCAGACCCUCCCCGCCGCCCCGGUCACCACUGCGGCAAUGGCCGAC
CCAGCUGCGGACUUGAUCGCGCCUAAGACUUAUUGCGAGGAACUGAAGGGCACCGACACUGGUC
AAGCGUGCCAGAUUCAGAUGUCCGACCCUGCCUACAACAUUAACAUCUCCCUGCCGUCGUACUA
UCCGGACCAAAAGUCGCUCGAGAACUACAUCGCCCAGACCCGCGAUAAGUUUCUUUCGGCCGCG
ACCUCCAGCACUCCGAGGGAGGCGCCAUACGAACUCAAUAUCACUAGCGCGACCUACCAGUCCG
CCAUCCCUCCGCGGGGUACCCAGGCCGUGGUGCUCAAGGUGUACCAGAACGCCGGAGGCACUCA
CCCUACCACCACCUACAAGGCCUUCGACUGGGACCAGGCGUACCGCAAGCCCAUUACCUAUGAU
ACGCUGUGGCAGGCCGACACCGAUCCUCUGCCGGUGGUGUUCCCAAUCGUGCAGGGCGAACUCA
GCAAGCAGACCGGGCAACAAGUCUCCAUCGCUCCCAACGCUGGCCUGGACCCCGUGAACUACCA
GAACUUCGCCGUGACCAACGACGGGGUCAUCUUCUUCUUCAACCCUGGAGAACUGCUGCCUGAA
GCCGCUGGACCUACUCAAGUGCUGGUGCCCCGCUCGGCAAUCGACUCCAUGCUCGCCGCCGAGA
CUGAUACAGAACAGGAGAUCGCCCUGUUCACUGUGACCAACCCACCCAGAACCGUGUCAGUGUC
AACUCUCAUGGACGGAAGGAUUGAUCAUGUGGAGCUGUCCGCGCGGGUGGCCUGGAUGAGCGAA
UCCCAGCUGGCCUCCGAAAUCCUCGUGAUCGCCGACCUGGCUCGACAGAAGGCCCAGUCCGCAC
AGUACGCCUUCAUUCUCGACCGGAUGAGCCAACAGGUCGACGCGGAUGAGCAUAGGGUGGCCCU
CCUGGGCUGGACUGCGGAUCCGAUCAUCGGCGUCCAAGUCCGCGCCGAUCGCAUCCUGGCUCUU
GCCGUGGCCGCCUCCAACAACCCUGAACUGACCACCUUGACGGCCGCCCUGUCGGGCCAACUCA
AUCCUCAAGUGAACUUGGUGGACACGCUGAACUCCGGGCAGUACACCGUGUUUGCCCCUACUAA
CGCCGCUUUCUCAAAACUCCCGGCCUCCACUAUUGACGAGCUCAAAACCAACUCCAGCCUGUUG
ACUUCCAUUUUGACCUACAAUCACAUUAUGCCGGGCGAACCGAACGUGGCCGUGAAGGAUCUCG
UCAGACACUUCGAACAACAGGUCCAACCGGGACGGGUGGUCGUGAUGCCUUGGGACCGGCAUAU
UGCGGCUGGAACCGAAAUCUCCCUGGAUCUGCUUGACCCUAUAUACAAGCGGAAGGUCCUGGAA
CUGGCUGCCGCGCUGCAAGCCGAAACCGCCGUGAACACCCUGUUUGAAAAGUUGGAGCCCAUGG
CAGCGGCGUCAAAGCCCCCGACCCCGCCUAUGGUGCACGCAGCCAGCACCAGACAGGCACUGAG
GGCACACUUCGCCGACGGUUGGAACACUUUCAACUUGGCCGAGGCCGAAGUGUUCGCCACUCGG
UACAGCGCUGCCAAGAACGCCGCGCAGCAGCUGGUGCUGUCCGCCGACAACAUGCGCGAGUACC
UGGCCGCGGGUGCAAAGGAAAGGCAGCGGCUCGCCACCAGUCUGCGCAACGCCGCCAAGCGGCU
GUACGCGGAGAAUCCGUCCGCCCGCGAUCAGAUCCUCCCGGUGUACGCUGAAUACCAGCAGCGG
UCCGAGAAAGUGCUUACCGAGUACAACAACAAGGCCGCCCUCGACCAGCAACGGCAAUGGAUCC
UGCAUAUGGCCAAACUGAGCGCCGCCAUGGCAAAGCAGGCUCAGUACGUGGCCCAGCUGCACGU
CUGGGCGCGGAGAGAGCACCCAACCUACGAAGGGGUGCACACUGCAAACGCCACUGUGUAUAUG
AUUGACACCGUGCUCAUGGCUUCAAAGGGCACCACGACCAAGAAGUACAUGGCAGCCGAUUACG
ACAAGCUGUUCCGGGCCACUAUCGCGGACGUGCUCGCCGAGAAGGAGCUAAGCCAUUACAAUGA
CAUCCGGGCCCAUACCUCCGUGAACGCAGUGAACCUGGAAGUGCUUCCUGCCCCCGAAUACUCG
UCGGCUCAGCGGGCCCUGUCAGACGCCGAUUGGCACUUUAUAGCCGAUCCAGCCUCCCGCUUCU
ACAACCUGGUCCUGGCGUUCGUGCGGCCCGUGGCCGUCGAUCUCACCUACAUUCCGGUGGUCGG
UCAUGCGCUCAGCGCAUCACAGUUCAACGACACCCUGAAUGUGUAUCUGACCGCCCACAACGCC
CUGGGGUCCAGCCUGCACACCGCCGGAGUGGACCUCGCCAAGUUUUUCGAUCCGCUGACCCGGG
GCGUGCUGACAAGAACCCCACUGAUGUCCCAGCUGAUUUUCAUUAUCGACCCCACUAUCUCCGC
GAUCGACGGACUCUACGACCUUUUGGGAAUCGGCAUCCCGAACCAGGGUGGCAUCCUCUACUCU
UCACUCGAGUAUUUCGAAAAAGCCCUGGAGGAGCUGGCCGCAGAACAAGUCGGCGGGCAGUCAC
AGCUCGGCAAACUGAACCCUGACGUCAACCUCGUGGAUACCCUCAACGGGGGAGAAUACACCGU
UUUCGCGCCCACUAACGCAGCUUUCGAUAAGCUGCCGGCUGCCACCAUUGACCAGCUCAAGACU
GACGCUAAGCUUCUGUCGUCGAUCCUGACCUACCACGUCAUCGCGGGCCAGGCCUCCCCCUCGC
GCAUCGACGGAACCCAUCAGACCCUAAGAAGAACUGCCCCGGCCCCGCCCUGGGCCAAGAUGAU
GGCCGUGGUCGGGGGAGCUCUGGCCUACCUGGUGGUCAAGACCCUGAUCAAUGCCACCCAACUG
CUGAAGCUGCUCGCAAAGCUGGGGGUGUCCACCGCCAACGCGACCGUGUACAUGAUCGAUAGCG
UGCUGAUGCAGCCUUUCUUUGACCCUUCCGCCUCCUUCCCCGGAAUCGUGGCUGGCCUUGCCGU
GUUGGCCGUCGUCGUGAUUGGCGCUGUGGUGGCUGCUGUGAUGUGUCGCCGCAAGAGCUCGGGG
GGAAAGGGAGGAAGCUAUUCCCAAGCGGCCUGCUCGGACAGCGCUCAGGGCUCGGACGUGUCGC
UGACCGCGUGAUAAUAGGCUCGCUUUCUUGCUGUCCAAUUUCUAUUAAAGGUUCCUUUGUUCCC
UAAGUCCAACUACUAAACUGGGGGAUAUUAUGAAGGGCCUUGAGCAUCUGGAUUCUGCCUAAUA
AAAAACAUUUAUUUUCAUUGCAA

TABLE 62
Physicochemical properties and characterization of HLA Class I and HLA
Class II Mtb mRNA DPPS-targeted KC3-OA LNPs with varying PL/Chol ratios,
described in terms of PL/Chol mol %/mol % ratio. All mRNAs used N1-methylpseudouridine
(N1MeU) instead of uridine (U) unless specified.

| | | PL | Particle | Particle Size | | | |
|---|---|---|---|---|---|---|---|
| | | Content, | Size | (nm) | | Zeta | Zeta |
| ICL | mRNA | mol % | (nm) | Post-F/T | E.E, % | pH 5 | pH 7 |
| KC3- | SEQ ID NOs. 223/224 | 25 | 101.40 | 99.79 | 90.0 ± 5.8 | 23.67 | 0.56 |
| OA | (unmodified uridine) | | | | | | |
| KC3- | SEQ ID NOs. 223/224 | 10 | 114.60 | 116.50 | 96.9 ± 6.3 | 17.39 | −0.02 |
| OA | | | | | | | |
| KC3- | SEQ ID NOs. 223/224 | 15 | 105.60 | 105.10 | 93.4 ± 4.1 | 17.49 | −1.63 |
| OA | | | | | | | |
| KC3- | SEQ ID NOs. 223/224 | 20 | 101.60 | 102.60 | 91.0 ± 6.6 | 18.61 | −0.91 |
| OA | | | | | | | |
| KC3- | SEQ ID NOs. 223/224 | 25 | 104.50 | 104.30 | 90.1 ± 2.8 | 20.96 | −1.43 |
| OA | | | | | | | |
| KC3- | SEQ ID NOs. 223/224 | 30 | 109.40 | 104.30 | 90.6 ± 6.2 | 25.48 | −1.45 |
| OA | | | | | | | |
| KC3- | SEQ ID NOs. 223/224 | 40 | 131.90 | 132.50 | 89.1 ± 4.7 | 29.1 | 1.07 |
| OA | | | | | | | |
| KC3- | SEQ ID NOs. 212/213 | 10 | 116.80 | 117.10 | 97.1 ± 13.5 | 19.13 | −0.06 |
| OA | | | | | | | |
| KC3- | SEQ ID NOs. 212/213 | 20 | 104.20 | 102.60 | 90.7 ± 5.5 | 20.07 | −7.41 |
| OA | | | | | | | |
| KC3- | SEQ ID NOs. 212/213 | 25 | 103.40 | 105.10 | 90.6 ± 8.0 | 23.02 | −1.74 |
| OA | | | | | | | |
| KC3- | SEQ ID NOs. 212/213 | 30 | 108.20 | 108.40 | 91.2 ± 3.9 | 27.89 | −1.06 |
| OA | | | | | | | |
| KC3- | SEQ ID NOs. 215/216 | 10 | 116.70 | 115.50 | 95.0 ± 4.6 | 19.84 | −1.04 |
| OA | | | | | | | |
| KC3- | SEQ ID NOs. 215/216 | 20 | 105.40 | 103.60 | 90.3 ± 4.1 | 18.64 | −0.98 |
| OA | | | | | | | |
| KC3- | SEQ ID NOs. 215/216 | 25 | 104.90 | 105.90 | 90.5 ± 3.0 | 24.27 | −1.50 |
| OA | | | | | | | |
| KC3- | SEQ ID NOs. 215/216 | 30 | 106.60 | 107.00 | 91.2 ± 8.3 | 26.65 | −1.14 |
| OA | | | | | | | |

KC3-OA LNP formulations containing between 10 and 40 mol % DSPC and 45.5 and 10.5 mol % cholesterol, respectively, all formed LNPs of similar particle size, encapsulation efficiency (E.E, %), and zeta potential. The highest mol % PL formulation (40 mol %) did show a slightly increased particle size of 132.5 nm, which was consistent with the results observed with mCherry mRNA in Example 39.

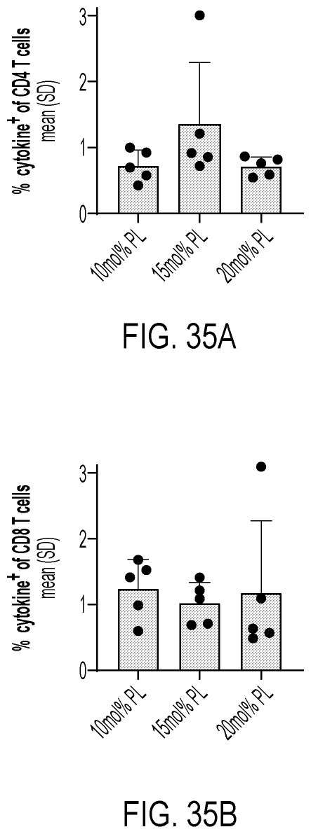

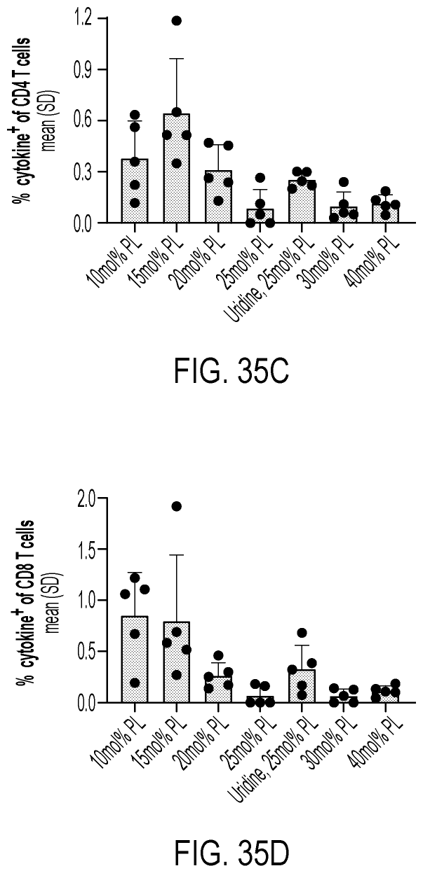

CB6F1 mice were immunized i.m. with 3 μg of a 2^{nd} generation HLA-II (SEQ ID NOS. 223 and 224) formulated in LNP formulations shown in Table 62. The ICL and DPPS were kept constant at 48 mol % and 5 mol %, respectively. PL content ranged from 10 to 40 mol %. Mice were boosted 4 weeks later and spleens were collected 7 days later. Splenocytes were stimulated with peptide pools covering each Mtb protein (1-2 peptide pools per well). Antigen-specific CD4 and CD8 T cells were defined as cells that produced either IFN-γ, TNF-α, IL-2, or combinations thereof. The magnitude of the CD4 (FIG. 35A) and CD8 (FIG. 35B) T cell responses were comparable between 10 and 20 mol %. This was somewhat unexpected given the results from the mCherry in vitro data (Table 61) where we found that mCherry levels peaked at 15 mol % PL and decreased at 20 mol % PL and greater.

Using slightly different stimulation conditions where all peptide pools covering Mtb protein antigens were combined in a single well, CD4 and CD8 T cell responses were quantified for LNP formulations containing 10 to 40 mol % PL. All mRNA was produced using N1-methylpseudouridine with exception of a 25 mol % PL group where the mRNA was produced using unmodified uridine. Under these stimulation conditions, the CD4 T cell response trended upward in the 15 mol % PL group, although this was not statistically different than the 10 mol % and 20 mol % PL groups (FIG. 35C). CD4 responses decreased when mRNA was formulated in LNPs with 25 mol % PL or greater, indicating that the optimal LNP composition consists of 10-20 mol % PL. mRNA using uridine rather than N1-methylpseudouridine was more immunogenic at 25 mol % PL. This is unexpected given that unmodified mRNA has been shown to inhibit mRNA translation caused by signaling through TLR3, TLR7 and TLR8 (Karikó et al. Immunity. 2005 August; 23(2):165-75; Karikó et al. Mol Ther. 2008 November; 16(11):1833-40). CD8 T cell responses followed a trend similar to CD4 T cells, although 20 mol % PL appeared to be less optimal (FIG. 35D). Altogether, we unexpectedly found we could further optimize in vitro transfection efficiency by increasing the DSPC mol % content from 10 to 15 mol % PL, and through the use of unmodified mRNA. In some embodiments, the LNP composition comprises 10-15 mol % PL and unmodified HLA Class I and HLA Class II Mtb mRNA.

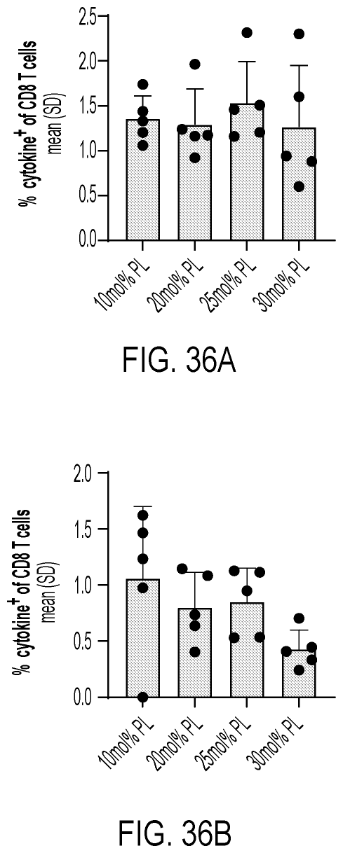

In a separate study, CB6F1 mice were immunized i.m. with 3 μg of a two different versions of a 2nd generation HLA-I Mtb mRNA. The first mRNA encoded for a mixture of Mtb antigens expressed only by Mtb or by both Mtb and BCG (referred to as “Mixed”), and a nucleotide sequence encoding for the sec signal peptide at the 5′ end and the MITD at the 3′ end (SEQ ID NOs. 212 and 213, Table 50). The second mRNA encoded for antigens expressed only by Mtb and not BCG (referred to as “Mtb-only”) and used the same flanking sec and MITD sequences (SEQ ID NOs. 215 and 216, Table 48). mRNA was encapsulated into LNPs using a range of 10 to 30 mol % PL. Mice were boosted 4 weeks later; spleens were harvested 8 days later and splenocytes were stimulated with overlapping peptide pools covering the protein antigen. CD8 T cell responses in mice immunized with the “Mixed” mRNA were similar between all LNP compositions (FIG. 36A). Similar results were seen with the “Mtb-only” mRNA-LNP compositions, although the magnitude of the CD8 response trended downward at 30 mol % (FIG. 36B). This study supported the findings in shown in FIGS. 35A-D that higher levels of DSPC phospholipid can be incorporated into LNPs. In this case, mRNA-LNPs were comparably immunogenic up to 30 mol % DSPC.

### Example 41. Protein Sequences for Mtb Genes Used in the Construction of mRNA Vaccine Constructs

SEQ ID NO. 229. Mycobacterium tuberculosis H37Rv | Rv0288 | esxH | TB10.4
MSQIMYNYPAMLGHAGDMAGYAGTLQSLGAEIAVEQAALQSAWQGDTGITYQAWQAQWNQAMEDLV
RAYHAMSSTHEANTMAMMARDTAEAAKWGG
SEQ ID NO. 230. Mycobacterium tuberculosis H37Rv | Rv1886c | fbpB | Ag85b
MTDVSRKIRAWGRRLMIGTAAAVVLPGLVGLAGGAATAGAFSRPGLPVEYLQVPSPSMGRDIKVQFQSGG
NNSPAVYLLDGLRAQDDYNGWDINTPAFEWYYQSGLSIVMPVGGQSSFYSDWYSPACGKAGCQTYKWET
FLTSELPQWLSANRAVKPTGSAAIGLSMAGSSAMILAAYHPQQFIYAGSLSALLDPSQGMGPSLIGLAMGDA
GGYKAADMWGPSSDPAWERNDPTQQIPKLVANNTRLWVYCGNGTPNELGGANIPAEFLENFVRSSNLKF
QDAYNAAGGHNAVFNFPPNGTHSWEYWGAQLNAMKGDLQSSLGAG
SEQ ID NO. 231. Mycobacterium tuberculosis H37Rv | Rv1196 | PPE18 | Mtb39a
MVDFGALPPEINSARMYAGPGSASLVAAAQMWDSVASDLFSAASAFQSVVWGLTVGSWIGSSAGLMVA
AASPYVAWMSVTAGQAELTAAQVRVAAAAYETAYGLTVPPPVIAENRAELMILIATNLLGQNTPAIAVNEAE
YGEMWAQDAAAMFGYAAATATATATLLPFEEAPEMTSAGGLLEQAAAVEEASDTAAANQLMNNVPQAL
QQLAQPTQGTTPSSKLGGLWKTVSPHRSPISNMVSMANNHMSMTNSGVSMTNTLSSMLKGFAPAAAAQ
AVQTAAQNGVRAMSSLGSSLGSSGLGGGVAANLGRAASVGSLSVPQAWAAANQAVTPAARALPLTSLTSA
AERGPGQMLGGLPVGQMGARAGGGLSGVLRVPPRPYVMPHSPAAG
SEQ ID NO. 232. Mycobacterium tuberculosis H37Rv | Rv3619c | esxV
MTINYQFGDVDAHGAMIRAQAGSLEAEHQAIISDVLTASDFWGGAGSAACQGFITQLGRNFQVIYEQANA
HGQKVQAAGNNMAQTDSAVGSSWA
SEQ ID NO. 233. Mycobacterium tuberculosis H37Rv | Rv3620c | esxW
MTSRFMTDPHAMRDMAGRFEVHAQTVEDEARRMWASAQNISGAGWSGMAEATSLDTMTQMNQAFR
NIVNMLHGVRDGLVRDANNYEQQEQASQQILSS
SEQ ID NO. 234. Mycobacterium tuberculosis H37Rv | Rv3874 | esxB | CFP10
MAEMKTDAATLAQEAGNFERISGDLKTQIDQVESTAGSLQGQWRGAAGTAAQAAVVRFQEAANKQKQEL
DEISTNIRQAGVQYSRADEEQQQALSSQMGF
SEQ ID NO. 235. Mycobacterium tuberculosis H37Rv | Rv3875 | esxA | ESAT-6
MTEQQWNFAGIEAAASAIQGNVTSIHSLLDEGKQSLTKLAAAWGGSGSEAYQGVQQKWDATATELNNAL
QNLARTISEAGQAMASTEGNVTGMFA
SEQ ID NO. 236. Mycobacterium tuberculosis H37Rv | Rv1788 | PE18
MSFVTTQPEALAAAAGSLQGIGSALNAQNAAAATPTTGVVPAAADEVSALTAAQFAAHAQIYQAVSAQAA
AIHEMFVNTLQMSSGSYAATEAANAAAAG
SEQ ID NO. 237. Mycobacterium tuberculosis H37Rv | Rv3804c | fbpA
MQLVDRVRGAVTGMSRRLVVGAVGAALVSGLVGAVGGTATAGAFSRPGLPVEYLQVPSPSMGRDIKVQF
QSGGANSPALYLLDGLRAQDDFSGWDINTPAFEWYDQSGLSVVMPVGGQSSFYSDWYQPACGKAGCQTY
KWETFLTSELPGWLQANRHVKPTGSAVVGLSMAASSALTLAIYHPQQFVYAGAMSGLLDPSQAMGPTLIGL
AMGDAGGYKASDMWGPKEDPAWQRNDPLLNVGKLIANNTRVWVYCGNGKPSDLGGNNLPAKFLEGFV
RTSNIKFQDAYNAGGGHNGVFDFPDSGTHSWEYWGAQLNAMKPDLQRALGATPNTGPAPQGA
SEQ ID NO. 238. Mycobacterium tuberculosis H37Rv | Rv1387 | PPE20
MTEPWIAFPPEVHSAMLNYGAGVGPMLISATQNGELSAQYAEAASEVEELLGVVASEGWQGQAAEAFVA
AYMPFLAWLIQASADCVEMAAQQHVVIEAYTAAVELMPTQVELAANQIKLAVLVATNFFGINTIPIAINEAEY
VEMWVRAATTMATYSTVSRSALSAMPHTSPPPLILKSDELLPDTGEDSDEDGHNHGGHSHGGHARMIDNF
FAEILRGVSAGRIVWDPVNGTLNGLDYDDYVYPGHAIWWLARGLEFFQDGEQFGELLFTNPTGAFQFLLYV
VVVDLPTHIAQIATWLGQYPQLLSAALTGVIAHLGAITGLAGLSGLSAIPSAAIPAVVPELTPVAAAPPMLAVA
GVGPAVAAPGMLPASAPAPAAAAGATAAGPTPPATGFGGFPPYLVGGGGPGIGFGSGQSAHAKAAASDS
AAAESAAQASARAQARAARRGRSAAKARGHRDEFVTMDMGFDAAAPAPEHQPGARASDCGAGPIGFAG
TVRKEAVVKAAGLTTLAGDDFGGGPTMPMMPGTWTHDQGVFDEHR
SEQ ID NO. 239. Mycobacterium tuberculosis H37Rv | Rv3020c | esxS
MSLLDAHIPQLIASHTAFAAKAGLMRHTIGQAEQQAMSAQAFHQGESAAAFQGAHARFVAAAAKVNTLLD
IAQANLGEAAGTYVAADAAAASSYTGF
SEQ ID NO. 240. Mycobacterium tuberculosis H37Rv | Rv1047 | Rv1047
MTSSHLIDAEQLLADQLAQASPDLLRGLLSTFIAALMGAEADALCGAGYRERSDERSNQRNGYRHRDFDTRA
ATIDVAIPKLRQGSYFPDWLLQRRKRAERALTSVVATCYLLGVSTRRMERLVETLGVTKLSKSQVSIMAKELDE
AVEAFRTRPLDAGPYTFLAADALVLKVREAGRVVGVHTLIATGVNAEGYREILGIQVTSAEDGAGWLAFFRDL
VARGLSGVALVTSDAHAGLVAAIGATLPAAAWQRCRTHYAANLMAATPKPSWPWVRTLLHSIYDQPDAES
VVAQYDRVLDALTDKLPAVAEHLDTARTDLLAFTAFPKQIWRQIWSNNPQERLNREVRRRTDVVGIFPDRAS
IIRLVGAVLAEQHDEWIEGRRYLGLEVLTRARAALTSTEEPAKQQTTNTPALTT
SEQ ID NO. 241. Mycobacterium tuberculosis variant bovis BCG | GenBank
ALA77657.1 | Rv1195 | PE13
MHVSFVMAYPEMLAAAADTLQSIGATTVASNAAAAAPTTGVVPPAADEVSALTAAHFAAHAAMYQSVSA
RAAAIHDQFVATLASSASSYAATEVANAAAAS
SEQ ID NO. 242. Mycobacterium tuberculosis H37Rv | Rv2031c | hspX
MATTLPVQRHPRSLFPEFSELFAAFPSFAGLRPTFDTRLMRLEDEMKEGRYEVRAELPGVDPDKDVDIMVRD
GQLTIKAERTEQKDFDGRSEFAYGSFVRTVSLPVGADEDDIKATYDKGILTVSVAVSEGKPTEKHIQIRSTN
SEQ ID NO. 243. Mycobacterium tuberculosis H37Rv | Rv3330 | dacB1
MAFLRSVSCLAAAVFAVGTGIGLPTAAGEPNAAPAACPYKVSTPPAVDSSEVPAAGEPPLPLVVPPTPVGGN
ALGGCGIITAPGSAPAPGDVSAEAWLVADLDSGAVIAARDPHGRHRPASVIKVLVAMASINTLTLNKSVAGT
ADDAAVEGTKVGVNTGGTYTVNQLLHGLLMHSGNDAAYALARQLGGMPAALEKINLLAAKLGGRDTRVAT
PSGLDGPGMSTSAYDIGLFYRYAWQNPVFADIVATRTFDFPGHGDHPGYELENDNQLLYNYPGALGGKTGY
TDDAGQTFVGAANRDGRRLMTVLLHGTRQPIPPWEQAAHLLDYGFNTPAGTQIGTLIEPDPSLMSTDRNPA
DRQRVDPQAAARISAADALPVRVGVAVIGALIVFGLIMVARAMNRRPQH

### Example 42. Identification of T Cell Epitopes from Mtb and Mtb Antigens

A lipid nanoparticle (LNP) composition can comprise an ionizable cationic lipid, sterol, one or more phospholipids comprising at least one anionic phospholipid, a conjugated lipid and one or more nucleic acid sequence (a) encoding a T cell epitope from Mycobacterium tuberculosis (Mtb), and/or (b) encoding Mycobacterium tuberculosis antigens recognized by T cells.

### Identification of Novel and Protective Mtb Antigens.

The Mtb genome encodes for approximately 4000 open reading frames (4.4 Megabases). Consequently, a challenge in TB vaccine development, outside the use of live attenuated Mtb or related strains (e.g. BCG), is the identification of a subset of antigens that would provide protective cellular immunity.

To identify additional antigens to include in an mRNA vaccine, T cells from latently infected people can be screened for reactivity against Mtb antigens. Due to ease of access, circulating T cells are isolated from blood. Multiple approaches have then been developed to identify T cell clones with T cell receptors (TCRs) specific to peptides bound to HLA molecules. In general, this can be approached from two different directions: one being the screening of antigens to identify T cells that subsequently become activated, and two being the identification of clonal TCRs uniquely present in Mtb-infected individuals and then working back to identify that TCR's cognate antigen.

### Screening of Mtb Antigens for the Identification of Reactive T Cells.

One approach is to culture T cells with overlapping peptide pools (e.g. 15mer peptides overlapping by 10-11 amino acids) that cover Mtb proteins. This approach can be used to identify both Mtb-specific CD4 and CD8 T cells. After ex vivo stimulation, T cells are then screened for signs of activation—this can either be a functional readout for cytokine/chemokine production that is readout using standard tools such as flow cytometry or ELISpot; the upregulation of activation-induced cell surface markers such as CD69, CD134, CD137, or CD154; or by the detection of other signaling pathways downstream from TCR activation. This “antigen forward” approach has been commonly used to screen large numbers of Mtb proteins; however, by practicality, predictive peptide/HLA binding algorithms have been employed to screen and prioritize which peptides to synthesize. For HLA class II-restricted epitopes, current algorithms can capture ~50% of the actual T cell response, and other approaches described below have identified antigens overlooked by the overlapping peptide pool approach.

Reverse “TCR forward” approaches have been developed that use the TCR to identify novel immunogenic antigens. In one approach, a T cell library is created by isolating circulating memory T cells from individuals previously exposed to an infectious pathogen and expanding them in an unbiased manner using anti-CD3 antibody and supporting cytokines such as IL-2. Reporter antigen-presenting cell lines (e.g. a cell line engineered to express a cell surface molecule or fluorescent protein downstream of the T cell effector molecule granzyme B) expressing individual HLA alleles are transduced with a vector library containing fragments of coding sequences covering all full-length ORFs of the pathogen (e.g. sequences coding for polypeptide stretches of 100 amino acids or less that overlap each other). Expanded memory T cells are then co-incubated with reporter cells; antigen-reporting cells that upregulate the reporter molecule can be isolated using magnetic bead enrichment or fluorescence-activated cell sorting. Reporter⁺ cells are sequenced to identify the antigenic polypeptide and minimal peptide epitopes can be further defined by stimulating the T cell library with focused peptides.

A second TCR-forward approach is to use algorithms to cluster TCR sequences into specificity clusters. Most recently Mark Davis and colleagues at Stanford University created one such algorithm called GLIPH followed by an improved second-generation algorithm GLIPH2. To identify novel Mtb antigens, antigen-specific T cells from latent TB infected donors were identified by stimulating donor PBMCs with Mtb lysate, purifying T cells that upregulated activation markers, and then performing single-cell TCRα/β sequencing. Representative TCR clones from GLIPH2 TCR specificity clusters were then expressed in a reporter cell line with luciferase under the control of the NFAT response element downstream of TCR signaling. Antigen specificities for TCR clones can be efficiently identified by co-incubating TCR-transduced reporter cells with artificial antigen presenting cells that had pre-incubated with subsets of proteins covering the Mtb proteome. Targeted epitope discovery can then be conducted for any protein pools that induced a positive luciferase signal.

Methodology for identification of Mtb T cell antigens is not limited to these approaches and novel approaches are constantly being developed or refined. Regardless, we could use any of these complimentary approaches to further identify candidate Mtb antigens that could be included in an mRNA vaccine.

Screening of Novel Vaccine Antigens for Inclusion in an mRNA Vaccine.

After the identification of potential Mtb antigens that could generate protective memory T cells, these must be empirically tested in animal models and, ultimately, in human clinical trials. New mRNA constructs can be synthesized by taking the antigenic amino acid sequence, concatenating it with other antigens of interest, and adding other components such as signal peptides and transmembrane/cytoplasmic domains to direct post-translational modification and localization of antigen optimal for the type of desired immune response (i.e. humoral or cellular). The corresponding nucleic acid ORF is then codon optimized, UTRs are added to the 5′ and 3′ ends, and this sequence is cloned this into a plasmid for mRNA production. Purified mRNA is then encapsulated in LNPs to make a preclinical vaccine product. Protein expression can be verified using standard in vitro transfection.

mRNA-LNP candidates must be then tested for immunogenicity—the ability to prime CD4 and/or CD8 T cells— and then tested for efficacy in a suitable Mtb-infection animal model. The mouse model is the most convenient for screening immunogenicity, although this could be accomplished using other animal species. Mice are immunized either with a single injection or a series of prime/boosts. Lymphocytes are then isolated from secondary lymphoid tissue such as the spleen or lymph nodes, cells are stimulated with overlapping peptide pools covering the vaccine antigen (e.g. peptide pools consisting of 15mer peptides overlapping by 11 residues) or with purified recombinant protein. Functional T cell activation is then assessed by standard immunological methods such as flow cytometry (cytokine production, upregulation of activation markers, binding to peptide/MHC multimers), ELISA, or ELISpot. Success criteria are to observe T cell responses to all regions of the antigen encoded by the mRNA (e.g. T cells respond to peptide pools covering individual Mtb proteins encoded in the 5′, middle, and 3′ regions of the mRNA ORF. Since the ability to form a T cell response requires both the proper TCR sequence but also MHC class I and II alleles that can bind the peptides, T cell responses in mice with severely limited MHC alleles will be limiting compared to diverse human populations. Outbred mice can be used to address this limitation. Additionally, T cell recognition of the mRNA encoded antigen can be assessed in humans by stimulating T cells isolated from LTBI donors with autologous antigen presenting cells pulsed with antigen.

After confirming immunogenicity, multiple animal models can be used to test vaccine efficacy. For Mtb, this can be assessed at various levels, this highest being the prophylactic prevention of infection altogether (sterilizing immunity). But this may not be a reasonable outcome for a TB vaccine, and a highly successful vaccine could prevent the development of active disease. This could be achieved by inducing T cell immunity that limits Mtb growth in infected tissues, prevents dissemination between lung lobes or to other organs, or prevents heterologous Mtb infection upon secondary exposure. The most commonly used models to test efficacy are mice and non-human primates (NHPs). While the T cell responses in mice or NHPs will be to different epitopes than in humans, the assumption is that these animals capture the biology of Mtb infection-if Mtb antigen “X” is protective in infection models, then it has a greater chance of being protective in humans.

Improvements to an mRNA Vaccine Construct Outside of the Antigen.

There are multiple components outside of the mRNA antigen-coding region that are critical to the function of the vaccine, and this is an area of intense interest and rapid discovery. The untranslated regions (UTRs) are critical to the stability and overall protein expression as is the 5′ cap design and length of the poly(A) tail. There are multiple commonly used UTRs with these desirable attributes, but this is a component that could be improved upon. Additional approaches to the poly(A) tail such encoding the tail into the plasmid (versus post-transcriptional enzymatic addition) and introducing a nucleotide spacer within the poly(A) tail to improve fidelity and stability have been identified.

Another critical component that is unique to nucleic acid vaccines is the ability to direct nascently translated proteins to various subcellular compartments for antigen processing. For instance, for CD4 and CD8 T cell responses it may be advantageous to direct the antigen to the endosomal compartment. The MHC class I presentation pathway for intracellular proteins uses the proteasome to process polypeptides into short 8-11 residue peptides ideal for class I binding and presentation to CD8 T cells. B cell activation and antibody production may be best achieved by producing protein antigens that localize to the cell surface or are secreted. These aspects can be directed by building in nucleic acid sequences encoding signal peptides, transmembrane/cytoplasmic domains, secretion signals, and multimerization domains. These tools can be co-opted from other proteins or eukaryotic species.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

All publications, patents and patent applications referenced in this specification are incorporated herein by reference in their entirety for all purposes to the same extent as if each individual publication, patent or patent application were specifically indicated to be so incorporated by reference.

1.-63. (canceled)

64. An immunogenic lipid nanoparticle (LNP) composition comprising:
a mRNA polynucleotide sequence encoding one or more antigens encapsulated in a lipid nanoparticle comprising (a) one or more phospholipid(s) in a total amount of phospholipids of 5-20 mol % of the total lipid content of the LNP composition, the one or more phospholipid(s) comprising an anionic phospholipid selected from the group consisting of a phosphatidylserine (PS) or phosphatidylglycerol (PG) in a total amount of 2.5-10 mol % of the total lipid content of the LNP composition; (b) a sterol in a ratio of phospholipid to sterol of 0.25 to 0.42 and in a total amount of 0.5-50 mol % of the total lipid content of the LNP composition; (c) an ionizable cationic lipid at a N/P ratio of 3 to 8 relative to the mRNA polynucleotide in a total amount of 40-65 mol % of the total lipid content of the LNP composition; and (d) a PEG-conjugated lipid in a total amount of 0.5-2.5 mol % of the total lipid content of the LNP composition,
wherein the ionizable cationic lipid comprises a 1,3-dioxolane ring that is (a) gem-disubstituted with a pair of hydrocarbon tails, and (b) further substituted with a dialkyl amino alkyl head group having a pKa of 6.2-7.5;
the mRNA polynucleotide comprising one or more mRNA polynucleotide sequence(s) each encoding (i) a lysosome-associated membrane protein 1 (LAMP-1) signal peptide or a human HLA Class I signal peptide and (ii) a LAMP-1 or a MHC class I trafficking domain (MITD) transmembrane and cytoplasmic domain, each of (i) and (ii) operably linked to open reading frame(s) (ORF) of the mRNA polynucleotide.

65. The composition of claim 64, wherein:
a. the mRNA polynucleotide sequence encodes the signal peptide 5′ to the ORF and encodes the transmembrane and cytoplasmic domain 3′ to the ORF; and
b. the mRNA polynucleotide sequence further comprises a 5′ untranslated region (UTR) and 3′ UTR, a polyA tail of about 80 to about 140 nucleotides in length, and (i) a 5′ enzymatic or (ii) a 5′ clean cap;
c. the mRNA polynucleotide sequence is a chemically modified mRNA polynucleotide sequence, wherein the chemically modified mRNA polynucleotide sequence comprises N1-methylpseudouridine.

66. The composition of claim 65, wherein composition comprises the ionizable cationic lipid at a N/P ratio of 4 to 6 relative to the mRNA.

67. The composition of claim 66, wherein:
a. the mRNA polynucleotide sequence encoding the one or more antigens is preceded by a nucleotide sequence encoding ubiquitin with a G76A mutation; or
b. the mRNA polynucleotide sequence further comprises a G/P (gly-pro), G/S (gly-ser) or AAY spacer polynucleotide sequence between mRNA polynucleotide ORF regions encoding one or more Mtb antigens; and the mRNA polynucleotide sequence further encodes (a) a LAMP-1 signal peptide and a LAMP-1 transmembrane and cytoplasmic domain; or (b) a human HLA Class I signal peptide and a LAMP-1 transmembrane and cytoplasmic domain.

68. The composition of claim 67, wherein the mRNA polynucleotide sequence encodes both a CD4 T-cell epitope and a CD8 T-cell epitope from Mycobacterium tuberculosis (Mtb), or one or more Mtb antigens recognized by CD4 T-cells and a CD8 T-cells; and
a. the sterol consists of cholesterol;
b. the ionizable cationic lipid is present in the composition in a total amount of 46-54 mol % of a total lipid content of the LNP composition, and at a N/P ratio of 4 to 6 relative to the nucleic acid;
c. the one or more phospholipids are selected from the group consisting of: distearoylphosphatidylcholine (DSPC), hydrogenated soy phosphatidylcholine (HSPC), and dipalmitoylphosphatidylcholine (DPPC) or a combination thereof;
d. the one or more anionic phospholipids are selected from the group consisting of: a phosphatidyl-L-serine (PS) and a phosphatidylglycerol (PG) or a combination thereof, in a total amount of 2-8 mol % of the total lipid content of the LNP composition; and
e. the PEG-conjugated lipid selected from PEG (2000)-dimyristoylglycerol (PEG-DMG) and PEG (Mol. weight 2,000)-dimyristoylphosphatidylethanolamine (PEG-DMPE), or a combination thereof, in a total amount of 1-3.5 mol % of the total lipid content of the LNP composition.

69. The composition of claim 68, wherein the one or more phospholipids consists of
a. a phospholipids selected from the group consisting of: distearoylphosphatidylcholine (DSPC), hydrogenated soy phosphatidylcholine (HSPC), and dipalmitoylphosphatidylcholine (DPPC); and
b. an anionic phospholipid selected from the group consisting of: dipalmitoylphosphatidyl-L-serine (DPPS), or distearoylphosphatidyl-L-serine (DSPS), distearoylphosphatidylglycerol (DSPG), and dipalmitoyphosphatidylglycerol (DPPG).

70. The composition of claim 69, wherein the lipid nanoparticles consist of:
a. distearoylphosphatidylcholine (DSPC) in a total amount of 5 mol % of a total lipid content of the LNP composition;
b. distearoylphosphatidyl-L-serine (DSPS) in a total amount of 5 mol % of a total lipid content of the LNP composition;
c. the ionizable cationic lipid in a total amount of 52 mol % of a total lipid content of the LNP composition;
d. PEG(2000)-dimyristoylglycerol (PEG-DMG) in a total amount of 1.5 mol % of a total lipid content of the LNP composition; and
e. cholesterol in amount of 36.5 mol % of a total lipid content of the LNP composition.

71. The composition of claim 70, wherein the mRNA polynucleotide sequence is a single concatenated mRNA polynucleotide sequence open reading frame (ORF) encoding (i) a combination of three or more Mtb antigens containing CD8 and CD4 T-cell epitopes, and (ii) a LAMP-1 signal peptide or a human HLA Class I signal peptide and a LAMP-1 or MITD transmembrane and cytoplasmic domain, each operably linked to the Mtb epitopes ORF encoding the Mtb antigens.

72. The composition of claim 71, wherein the nucleic acid sequence encodes a concatenated sequence, wherein the concatenated nucleic acid-encoded sequence includes an N-terminal and C-terminal signal peptide selected from Sec/MITD, Lamp1, HLA-Dra, or tPA.

73. A method comprising administering to a subject in need thereof the composition of claim 70, in an amount effective to induce in the subject an immune response against Mycobacterium tuberculosis infection.
